(19) United States
(12) Patent Application Publication
(10) Pub. No.: US 2025/0090540 A1
(43) Pub. Date: Mar. 20, 2025

(54) PI3K-ALPHA INHIBITORS AND METHODS OF USE THEREOF

(71) Applicants: Relay Therapeutics, Inc., Cambridge, MA (US); D. E. Shaw Research, LLC, New York, NY (US)

(72) Inventors: Alessandro BOEZIO, Cambridge, MA (US); Alexander M. TAYLOR, Cambridge, MA (US); Hakan GUNAYDIN, Cambridge, MA (US); Hanmo ZHANG, Cambridge, MA (US); Kevin David RAYNOR, Cambridge, MA (US); Kelley C. SHORTSLEEVES, Cambridge, MA (US); Lucian V. DIPIETRO, Cambridge, MA (US); Levi Charles Thomas PIERCE, Cambridge, MA (US); Nicolas PABON, Cambridge, MA (US); Thomas H. MCLEAN, Cambridge, MA (US); Fabrizio GIORDANETTO, New York, NY (US); Yakov PECHERSKY, New York, NY (US); Qi WANG, New York, NY (US); Alexandre LARIVEE, Montreal (CA); Fei CHEN, Montreal (CA); Gaetan MAERTENS, Montreal (CA); Johanne OUTIN, Montreal (CA); Megan BERTRAND-LAPERLE, Montreal (CA); Mohan PAL, Montreal (CA); Sampada CHITALE, Montreal (CA); Michael Paul DENINNO, Gales Ferry, CA (US)

(21) Appl. No.: 18/689,692

(22) PCT Filed: Sep. 9, 2022
(86) PCT No.: PCT/US2022/076213

§ 371 Date: Mar. 6, 2024

Related U.S. Application Data
(60) Provisional application No. 63/261,082, filed on Sep. 10, 2021.

(51) Int. Cl. A61K 31/538; A61K 31/4184; A61K 31/4412; A61K 31/495; C07D 241/08; C07D 401/04; C07D 409/12; C07D 413/14

(52) U.S. Cl. CPC A61K 31/538; A61K 31/4184; A61K 31/4412; A61K 31/495; C07D 241/08; C07D 401/04; C07D 409/12; C07D 413/14

(57) ABSTRACT

The present disclosure relates to novel compounds and pharmaceutical compositions thereof, and methods for inhibiting the activity of PI3Kα enzymes with the compounds and compositions of the disclosure. The present disclosure further relates to, but is not limited to, methods for treating disorders associated with PI3Kα signaling with the compounds and compositions of the disclosure.

### CROSS-REFERENCE TO RELATED APPLICATIONS

This application claims the benefit of U.S. Provisional Application No. 63/261,082 filed on Sep. 10, 2021, the entirety of which is hereby incorporated by reference.

### BACKGROUND

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃), which, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Of the two Class 1 PI3K sub-classes, Class 1A PI3Ks are heterodimers composed of a catalytic p110 subunit (alpha, beta, or delta isoforms) constitutively associated with a regulatory subunit that can be p85 alpha, p55 alpha, p50 alpha, p85 beta, or p55 gamma. The Class 1B sub-class has one family member, a heterodimer composed of a catalytic p110 gamma subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997); Katso et al., Annu. Rev. Cell Dev. Biol. 17:615-675 (2001)).

Consequently, the resultant phospholipid products of Class I PI3Ks link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)). In many cases, PIP₂ and PIP₃ recruit Aid, the product of the human homologue of the viral oncogene v-Akt, to the plasma membrane where it acts as a nodal point for many intracellular signaling pathways important for growth and survival (Fantl et al., Cell 69:413-423 (1992); Bader et al., Nature Rev. Cancer 5:921 (2005); Vivanco and Sawyer, Nature Rev. Cancer 2:489 (2002)).

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

In view of the above, inhibitors of PI3Kα would be of particular value in the treatment of proliferative disease and other disorders. While multiple inhibitors of PI3Ks have been developed (for example, taselisib, alpelisib, buparlisib and others), these molecules inhibit multiple Class 1A PI3K isoforms. Inhibitors that are active against multiple Class 1A PI3K isoforms are known as “pan-PI3K” inhibitors. A major hurdle for the clinical development of existing PI3K inhibitors has been the inability to achieve the required level of target inhibition in tumors while avoiding toxicity in cancer patients. Pan-PI3K inhibitors share certain target-related toxicities including diarrhea, rash, fatigue, and hyperglycemia. The toxicity of PI3K inhibitors is dependent on their isoform selectivity profile. Inhibition of PI3Kα is associated with hyperglycemia and rash, whereas inhibition of PI3Kδ or PI3Kγ is associated with diarrhea, myelosuppression, and transaminitis (Hanker et al., Cancer Discovery (2019) PMID: 30837161. Therefore, selective inhibitors of PI3Kα may increase the therapeutic window, enabling sufficient target inhibition in the tumor while avoiding dose-limiting toxicity in cancer patients.

### SUMMARY

In some embodiments, the present disclosure provides a compound of formula I:

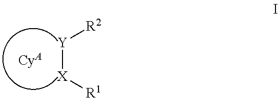

or a pharmaceutically acceptable salt thereof, wherein each of Cy^{A}, R¹, R², X, and Y is as defined in embodiments and classes and subclasses herein.

In some embodiments, the present disclosure provides a pharmaceutical composition comprising a compound of formula I, or a pharmaceutically acceptable salt thereof, and a pharmaceutically acceptable carrier, adjuvant, or diluent.

In some embodiments, the present disclosure provides a method of treating a PI3Kα-mediated disorder comprising administering to a patient in need thereof a compound of formula I, or composition comprising said compound.

In some embodiments, the present disclosure provides a process for providing a compound of formula I, or synthetic intermediates thereof.

In some embodiments, the present disclosure provides a process for providing pharmaceutical compositions comprising compounds of formula I.

### DETAILED DESCRIPTION

### 1. General Description of Certain Embodiments of the Disclosure

Compounds of the present disclosure, and pharmaceutical compositions thereof, are useful as inhibitors of PI3Kα. In some embodiments, the present disclosure provides a compound of formula I:

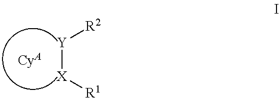

or a pharmaceutically acceptable salt thereof, wherein:
X is C, CH, C(R^{X}), or N;
Y is C, CH, C(R^{Y}), or N;
R¹ is -L¹-R^{1A};
R² is -L²-R^{2A};
R^{X} is -L^{X}-R^{XA};
R^{Y} is -L^{Y}-R^{YA};
each instance of R^{CyA} is independently -L^{CyA}-R^{CyAA};
Cy^{A} is a 5-6 membered saturated, partially unsaturated, or aromatic monocyclic ring having 0-3 heteroatoms independently selected from nitrogen, oxygen, and sulfur; or a 8-10 membered saturated, partially unsaturated, or aromatic bicyclic ring having 0-4 heteroatoms independently selected from nitrogen, oxygen, and sulfur; wherein each ring is substituted with n instances of R^{CyA};
each of L¹, L², L^{X}, L^{Y}, and L^{CyA} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{1A} is R^{A} or R^{B} substituted by r¹ instances of R^{1C};
R^{2A} is R^{A} or R^{B} substituted by r² instances of R^{2C};
R^{XA} is R^{A} or R^{B} substituted by r³ instances of R^{XC};
R^{YA} is R^{A} or R^{B} substituted by r⁴ instances of R^{YC};
R^{L} is R^{A} or R^{B} substituted by r⁵ instances of R^{LC};
each instance of R^{CyAA} is independently R^{A} or R^{B} substituted by r⁶ instances of R^{CyAC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{XC}, R^{YC}, R^{LC}, and R^{CyAC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
   each of n, r¹, r², r³, r⁴, r⁵, and r⁶ is independently 0, 1, 2, 3, 4, or 5.

### 2. Compounds and Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “C₁₋₈ (or C₁₋₆, or C₁₋₄) bivalent saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl,” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but is not limited to, phenyl, biphenyl, naphthyl, anthracyl and the like, which may bear one or more substituents.

The terms “heteroaryl” or “heteroaromatic”, unless otherwise defined, as used herein refers to a monocyclic aromatic 5-6 membered ring containing one or more heteroatoms, for example one to three heteroatoms, such as nitrogen, oxygen, and sulfur, or an 8-10 membered polycyclic ring system containing one or more heteroatoms, wherein at least one ring in the polycyclic ring system is aromatic, and the point of attachment of the polycyclic ring system is through a ring atom on an aromatic ring. A heteroaryl ring may be linked to adjacent radicals though carbon or nitrogen. Examples of heteroaryl rings include but are not limited to furan, thiophene, pyrrole, thiazole, oxazole, isothiazole, isoxazole, imidazole, pyrazole, triazole, pyridine, pyrimidine, indole, etc. For example, unless otherwise defined, 1,2,3,4-tetrahydroquinoline is a heteroaryl ring if its point of attachment is through the benzo ring, e.g.:

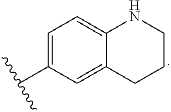 [SMILES: *c1ccc2c(c1)CCCN2]

The terms “heterocyclyl” or “heterocyclic group”, unless otherwise defined, refer to a saturated or partially unsaturated 3-10 membered monocyclic or 7-14 membered polycyclic ring system, including bridged or fused rings, and whose ring system includes one to four heteroatoms, such as nitrogen, oxygen, and sulfur. A heterocyclyl ring may be linked to adjacent radicals through carbon or nitrogen.

The term “partially unsaturated” in the context of rings, unless otherwise defined, refers to a monocyclic ring, or a component ring within a polycyclic (e.g. bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g.:

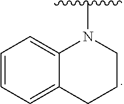 [SMILES: *N1CCCc2ccccc21]

The term “saturated” in the context of rings, unless otherwise defined, refers to a 3-10 membered monocyclic ring, or a 7-14 membered polycyclic (e.g. bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g.:

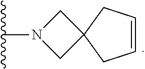 [SMILES: *N1CC2(CC=CC2)C1]

The terms “alkylene”, “arylene”, “cycloalkylene”, “heteroarylene”, “heterocycloalkylene”, and the other similar terms with the suffix “-ylene” as used herein refers to a divalently bonded version of the group that the suffix modifies. For example, “alkylene” is a divalent alkyl group connecting the groups to which it is attached.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

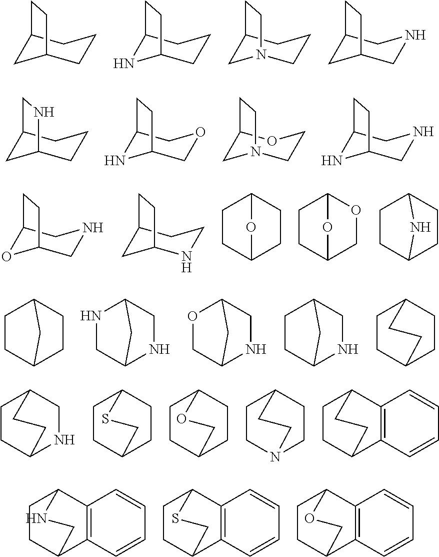

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄₀C(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)(OR^{∘})R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR•), —(CH₂)₀₋₂OH, —(CH₂)₀₋₂OR^{•}, —(CH₂)₀₋₂CH(OR•)₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR•, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of this disclosure. Therefore, unless otherwise stated, single stereochemical isomers as well as mixtures of enantiomeric, diastereomeric, and geometric (or conformational) isomers of the present compounds are within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations. Unless otherwise stated, all tautomers of the compounds of the disclosure are within the scope of the disclosure.

The term “isotopic substitution” as used herein refers to the substitution of an atom with its isotope. The term “isotope” as used herein refers to an atom having the same atomic number as that of atoms dominant in nature but having a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O. Unless otherwise stated, all isotopic substitution of the compounds of the disclosure are within the scope of the disclosure. Such compounds are useful, for example, as analytical tools, as probes in biological assays, or as therapeutic agents in accordance with the present disclosure. In certain embodiments, for example, a warhead moiety, R^{W}, of a provided compound comprises one or more deuterium atoms.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Exemplary pharmaceutically acceptable salts are found, e.g., in Berge, et al. (J. Pharm. Sci. 1977, 66(1), 1; and Gould, P. L., Int. J. Pharmaceutics 1986, 33, 201-217; (each hereby incorporated by reference in its entirety).

Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, e.g., to reduce or ameliorate the severity and/or duration of afflictions related to PI3Kα signaling, or one or more symptoms thereof, prevent the advancement of conditions or symptoms related to afflictions related to PI3Kα signaling, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to PI3Kα signaling activity or that may otherwise be relieved by the compounds and/or compositions of the disclosure.

### 3. Description of Exemplary Embodiments

As described above, in some embodiments, the present disclosure provides a compound of formula I:

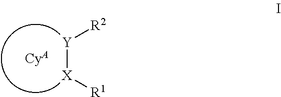

or a pharmaceutically acceptable salt thereof, wherein:
X is C, CH, C(R^{X}), or N;
Y is C, CH, C(R^{Y}), or N;
R¹ is -L¹-R^{1A};
R² is -L²-R^{2A};
R^{X} is -L^{X}-R^{XA};
R^{Y} is -L^{Y}-R^{YA}; or
each instance of R^{CyA} is independently -L^{CyA}-R^{CyAA};
Cy^{A} is a 5-6 membered saturated, partially unsaturated, or aromatic monocyclic ring having 0-3 heteroatoms independently selected from nitrogen, oxygen, and sulfur; or a 8-10 membered saturated, partially unsaturated, or aromatic bicyclic ring having 0-4 heteroatoms independently selected from nitrogen, oxygen, and sulfur; wherein each ring is substituted with n instances of R^{CyA};
each of L¹, L², L^{X}, L^{Y}, and L^{CyA} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{1A} is R^{A} or R^{B} substituted by r¹ instances of R^{1C};
R^{2A} is R^{A} or R^{B} substituted by r² instances of R^{2C};
R^{XA} is R^{A} or R^{B} substituted by r³ instances of R^{XC};
R^{YA} is R^{A} or R^{B} substituted by r⁴ instances of R^{YC};
R^{L} is R^{A} or R^{B} substituted by r⁵ instances of R^{LC};
each instance of R^{CyAA} is independently R^{A} or R^{B} substituted by r⁶ instances of R^{CyAC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{XC}, R^{YC}, R^{LC}, and R^{CyAC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, r¹, r², r³, r⁴, r⁵, and r⁶ is independently 0, 1, 2, 3, 4, or 5.

As defined generally above, X is C, CH, C(R^{X}), or N. In some embodiments, X is C. In some embodiments, X is CH. In some embodiments, X is C(R^{X}). In some embodiments, X is N. In some embodiments, X is CH or C(R^{X}). In some embodiments, X is CH or N. In some embodiments, X is C(R^{X}) or N. In some embodiments, X is selected from the groups depicted in the compounds in Table 1.

As defined generally above, Y is C, CH, C(R^{Y}), or N. In some embodiments, Y is C. In some embodiments, Y is CH. In some embodiments, Y is C(R^{Y}). In some embodiments, Y is N. In some embodiments, Y is CH or C(R^{Y}). In some embodiments, Y is CH or N. In some embodiments, Y is C(R^{Y}) or N. In some embodiments, Y is selected from the groups depicted in the compounds in Table 1.

As defined generally above, R¹ is -L¹-R^{1A}. In some embodiments, R¹ is -L¹-R^{1A}. In some embodiments, R¹ is —R^{1A}.

In some embodiments, R¹ (i.e. -L¹-R^{1A} taken together) is

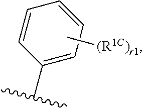

wherein R^{1C} and r¹ are as defined in the embodiments and classes and subclasses herein. In some embodiments, R¹ (i.e. -L¹-R^{1A} taken together) is

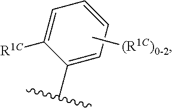

wherein R^{1C} is as defined in the embodiments and classes and subclasses herein. In some embodiments, R¹ (i.e. -L¹-R^{1A} taken together) is

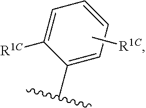

wherein R^{1C} is as defined in the embodiments and classes and subclasses herein. In some embodiments, R¹ (i.e. -L¹-R^{1A} taken together) is

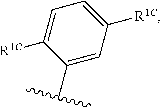

wherein R^{1C} is as defined in the embodiments and classes and subclasses herein. In some embodiments, R¹ (i.e. -L¹-R^{1A} taken together) is

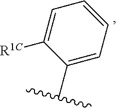

wherein R^{1C} is as defined in the embodiments and classes and subclasses herein.

In some embodiments, R¹ (i.e. -L¹-R^{1A} taken together) is

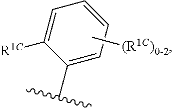

wherein each instance of R^{1C} is independently halogen, —CN, —O-(optionally substituted C₁₋₆ aliphatic), or an optionally substituted C₁₋₆ aliphatic. In some embodiments, R¹ (i.e. -L¹-R^{1A} taken together) is

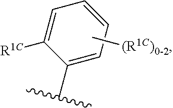

wherein each instance of R^{1C} is independently halogen or C₁₋₃ aliphatic optionally substituted with 1-3 halogen. In some embodiments, R¹ (i.e. -L¹-R^{1A} taken together) is

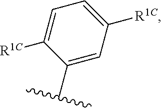

wherein each instance of R^{1C} is independently halogen or C₁₋₃ aliphatic optionally substituted with 1-3 halogen. In some embodiments, R¹ (i.e. -L¹-R^{1A} taken together) is

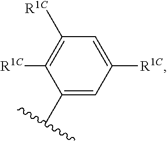

wherein each instance of R^{1C} is independently halogen or C₁₋₃ aliphatic optionally substituted with 1-3 halogen. In some embodiments, R¹ (i.e. -L¹-R^{1A} taken together) is

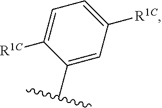

wherein each instance of R^{1C} is independently fluorine, chlorine, —CH₃, —CHF₂, or —CF₃. In some embodiments, R¹ (i.e. -L¹-R^{1A} taken together) is

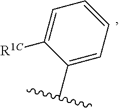

wherein R^{1C} is halogen or C₁₋₃ aliphatic optionally substituted with 1-3 halogen.

In some embodiments, R¹ (i.e. -L¹-R^{1A} taken together) is

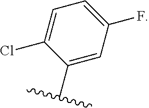 [SMILES: *c1cc(F)ccc1Cl]

In some embodiments, R¹ (i.e. -L¹-R^{1A} taken together) is

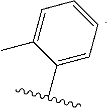 [SMILES: *c1ccccc1C]

In some embodiments, R¹ (i.e. -L¹-R^{1A} taken together) is

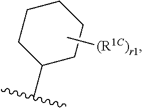

wherein R^{1C} and r¹ are as defined in the embodiments and classes and subclasses herein. In some embodiments, R¹ (i.e. -L¹-R^{1A} taken together) is

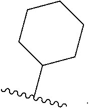 [SMILES: *C1CCCCC1]

In some embodiments, R¹ (i.e. -L¹-R^{1A} taken together) is

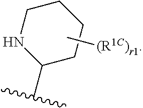

In some embodiments, R¹ (i.e. -L¹-R^{1A} taken together) is

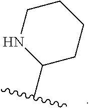 [SMILES: *C1CCCCN1]

In some embodiments, R¹ is selected from the groups depicted in the compounds in Table 1.

As defined generally above, R² is -L²-R^{2A}. In some embodiments, R² (i.e. -L²-R^{2A} taken together) is —N(R)C(O)—R^{2A}, —N(R)—R^{2A}, or —R^{2A}, wherein R and R^{2A} are as defined in the embodiments and classes and subclasses herein. In some embodiments, R² (i.e. -L²-R^{2A} taken together) is —N(R)C(O)—R^{2A} or —R^{2A}, wherein R and R^{2A} are as defined in the embodiments and classes and subclasses herein. In some embodiments, R² is —N(H)C(O)—R^{2A}, —N(H)—R^{2A}, or —R^{2A}.

In some embodiments, R² (i.e. -L²-R^{2A} taken together) is —N(R)C(O)—R^{2A}, wherein R and R^{2A} are as defined in the embodiments and classes and subclasses herein. In some embodiments, R² (i.e. -L²-R^{2A} taken together) is —N(H)C(O)—R^{2A}, wherein R^{2A} is as defined in the embodiments and classes and subclasses herein. In some embodiments, R² (i.e. -L²-R^{2A} taken together) is —N(H)C(O)—R^{2A}, wherein R^{2A} is R^{B} substituted by r² instances of R^{2C}. In some embodiments, R² (i.e. -L²-R^{2A} taken together) is —N(R)—R^{2A}, wherein R and R^{2A} are as defined in the embodiments and classes and subclasses herein. In some embodiments, R² is —R^{2A}.

In some embodiments, R² is —N(H)C(O)—R^{2A}, —N(H)C(O)N(H)—R^{2A}, —C(O)N(H)—R^{2A}, —N(H)—R^{2A}, —S(O)₂CH₂—R^{2A}, —CH₂S(O)₂—R^{2A}, or —C(H)(CH₃)OH. In some embodiments, R² is —N(H)C(O)—R^{2A}, —N(H)C(O)N(H)—R^{2A}, or —N(H)—R^{2A}. In some embodiments, R² is —C(O)N(H)—R^{2A}, —CH₂S(O)₂—R^{2A}, or —C(H)(CH₃)OH. In some embodiments, R² is —S(O)₂CH₂—R^{2A} or —CH₂S(O)₂—R^{2A}.

In some embodiments, R² is —N(H)C(O)N(H)—R^{2A}. In some embodiments, R² is —C(O)N(H)—R^{2A}. In some embodiments, R² is —N(H)—R^{2A}. In some embodiments, R² is —S(O)₂CH₂—R^{2A}. In some embodiments, R² is —CH₂S(O)₂—R^{2A}. In some embodiments, R² is —C(H)(CH₃)OH.

In some embodiments, R² (i.e. -L²-R^{2A} taken together) is

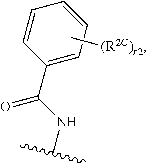

wherein R^{2C} and r² are as defined in the embodiments and classes and subclasses herein. In some embodiments, R² (i.e. -L²-R^{2A} taken together) is

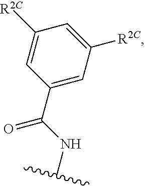

wherein R^{2C} is as defined in the embodiments and classes and subclasses herein.

In some embodiments, R² (i.e. -L²-R^{2A} taken together) is

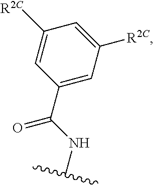

wherein each instance of R^{2C} is independently halogen, —CN, —O-(optionally substituted C₁₋₆ aliphatic), or an optionally substituted C₁₋₆ aliphatic. In some embodiments, R² (i.e. -L²-R^{2A} taken together) is

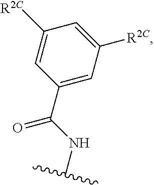

wherein each instance of R^{2C} is independently halogen or C₁₋₃ aliphatic optionally substituted with 1-3 halogen. In some embodiments, R² (i.e. -L²-R^{2A} taken together) is

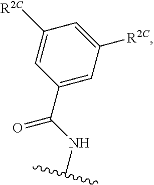

wherein each instance of R^{2C} is independently fluorine, chlorine, —CH₃, —CHF₂, or —CF₃. In some embodiments, R² (i.e. -L²-R^{2A} taken together) is

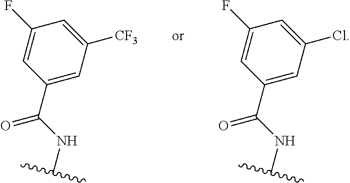 [SMILES: *NC(=O)c1cc(F)cc(C(F)(F)F)c1 ; *NC(=O)c1cc(F)cc(Cl)c1]

In some embodiments, R² (i.e. -L²-R^{2A} taken together) is

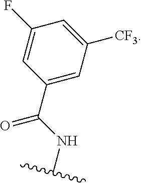 [SMILES: *NC(=O)c1cc(F)cc(C(F)(F)F)c1]

In some embodiments, R² (i.e. -L²-R^{2A} taken together) is

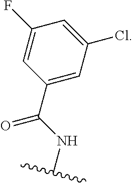 [SMILES: *NC(=O)c1cc(F)cc(Cl)c1]

In some embodiments, R² (i.e. -L²-R^{2A} taken together) is

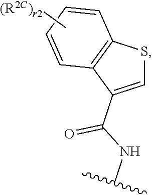

wherein R^{2C} and r² are as defined in the embodiments and classes and subclasses herein. In some embodiments, R² (i.e. -L²-R^{2A} taken together) is

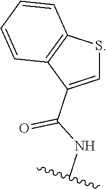 [SMILES: *NC(=O)c1csc2ccccc12]

In some embodiments, R² (i.e. -L²-R^{2A} taken together) is

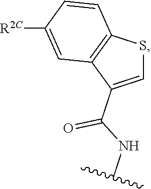

wherein R^{2C} is as defined in the embodiments and classes and subclasses herein.

In some embodiments, R² (i.e. -L²-R^{2A} taken together) is

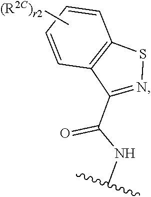

wherein R^{2C} and r² are as defined in the embodiments and classes and subclasses herein. In some embodiments, R² (i.e. -L²-R^{2A} taken together) is

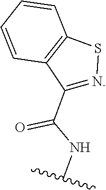 [SMILES: *NC(=O)c1nsc2ccccc12]

In some embodiments, R² (i.e. -L²-R^{2A} taken together) is

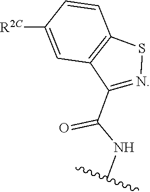

wherein R^{2C} is as defined in the embodiments and classes and subclasses herein.

In some embodiments, R² (i.e. -L²-R^{2A} taken together) is

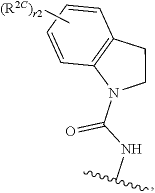

wherein R^{2C} and r² are as defined in the embodiments and classes and subclasses herein. In some embodiments, R² (i.e. -L²-R^{2A} taken together) is

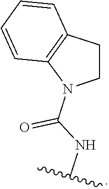 [SMILES: *NC(=O)N1CCc2ccccc21]

In some embodiments, R² (i.e. -L²-R^{2A} taken together) is

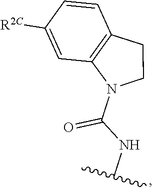

wherein R^{2C} is as defined in the embodiments and classes and subclasses herein.

In some embodiments, R² (i.e. -L²-R^{2A} taken together) is

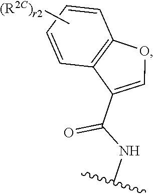

wherein R^{2C} and r² are as defined in the embodiments and classes and subclasses herein. In some embodiments, R² (i.e. -L²-R^{2A} taken together) is

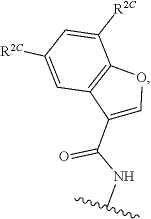

wherein R^{2C} is as defined in the embodiments and classes and subclasses herein.

In some embodiments, R² (i.e. -L²-R^{2A} taken together) is

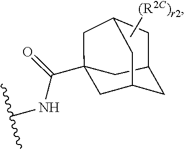

wherein R^{2C} and r² are as defined in the embodiments and classes and subclasses herein. In some embodiments, R² (i.e. -L²-R^{2A} taken together) is

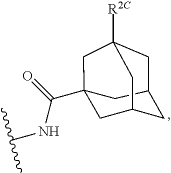

wherein R^{2C} is as defined in the embodiments and classes and subclasses herein.

In some embodiments, R² (i.e. -L²-R^{2A} taken together) is

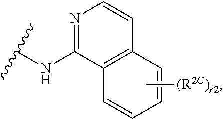

wherein R^{2C} and r² are as defined in the embodiments and classes and subclasses herein. In some embodiments, R² (i.e. -L²-R^{2A} taken together) is

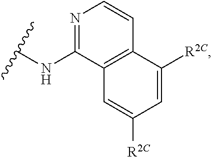

wherein R^{2C} is as defined in the embodiments and classes and subclasses herein.

In some embodiments, R² (i.e. -L²-R^{2A} taken together) is

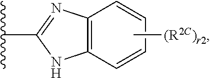

wherein R^{2C} and r² are as defined in the embodiments and classes and subclasses herein. In some embodiments, R² (i.e. -L²-R^{2A} taken together) is

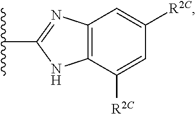

wherein R^{2C} is as defined in the embodiments and classes and subclasses herein.

In some embodiments, R² is

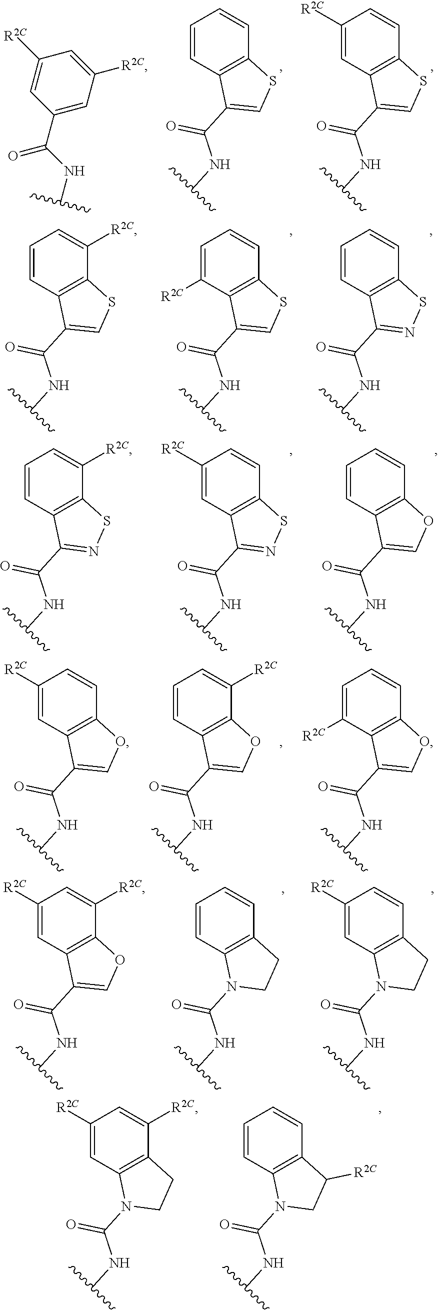

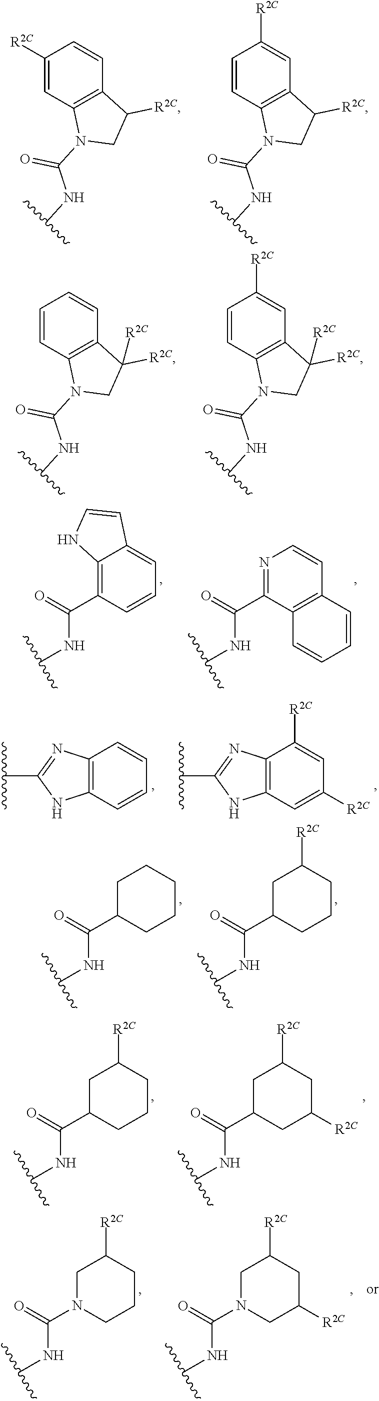

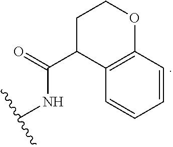 [SMILES: *NC(=O)C1CCOc2ccccc21]

In some embodiments, R² is

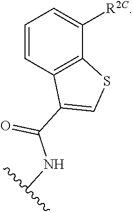

In some embodiments, R² is

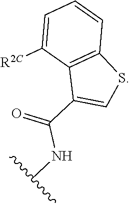

In some embodiments, R² is

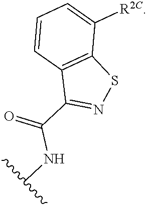

In some embodiments, R² is

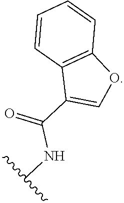 [SMILES: *NC(=O)c1coc2ccccc12]

In some embodiments, R² is

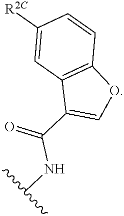

In some embodiments, R² is

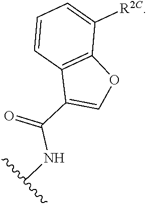

In some embodiments, R² is

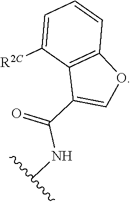

In some embodiments, R² is

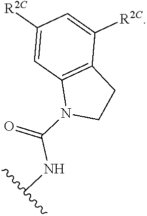

In some embodiments, R² is

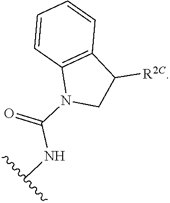

In some embodiments, R² is

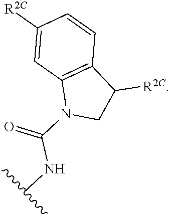

In some embodiments, R² is

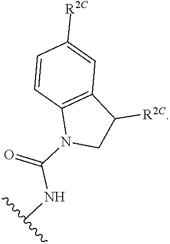

In some embodiments, R² is

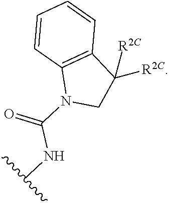

In some embodiments, R² is

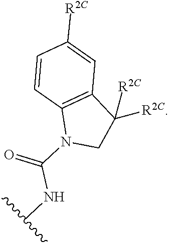

In some embodiments, R² is

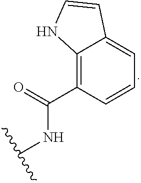 [SMILES: *NC(=O)c1cccc2cc[nH]c12]

In some embodiments, R² is

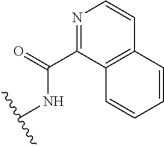 [SMILES: *NC(=O)c1nccc2ccccc12]

In some embodiments, R² is

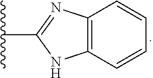 [SMILES: *c1nc2ccccc2[nH]1]

In some embodiments, R² is

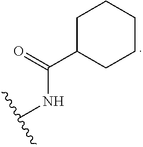 [SMILES: *NC(=O)C1CCCCC1]

In some embodiments, R² is

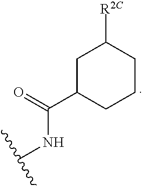

In some embodiments, R² is

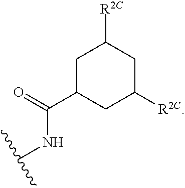

In some embodiments, R² is

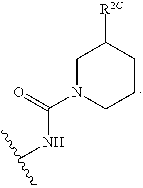

In some embodiments, R² is

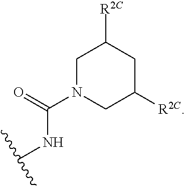

In some embodiments, R² is

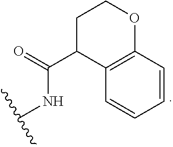 [SMILES: *NC(=O)C1CCOc2ccccc21]

In some embodiments, R² is selected from the groups depicted in the compounds in Table 1.

As defined generally above, R^{X} is -L^{X}-R^{XA}. In some embodiments, R^{X} is —R^{XA}.

In some embodiments, R^{X} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{X} is halogen, —CN, —OH, —O-(optionally substituted C₁₋₆ aliphatic), or an optionally substituted C₁₋₆ aliphatic. In some embodiments, R^{X} is halogen, —OH, or C₁₋₃ aliphatic optionally substituted with 1-3 halogen. In some embodiments, R^{X} is fluorine, chlorine, —OH, or —CH₃. In some embodiments, R^{X} is deuterium. In some embodiments, R^{X} is selected from the groups depicted in the compounds in Table 1.

As defined generally above, R^{Y} is -L^{Y}-R^{YA}. In some embodiments, R^{Y} is —R^{YA}.

In some embodiments, R^{Y} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Y} is halogen, —CN, —OH, —O-(optionally substituted C₁₋₆ aliphatic), or an optionally substituted C₁₋₆ aliphatic. In some embodiments, R^{Y} is halogen, —OH, or C₁₋₃ aliphatic optionally substituted with 1-3 halogen. In some embodiments, R^{Y} is fluorine, chlorine, —OH, or —CH₃. In some embodiments, R^{Y} is deuterium. In some embodiments, R^{Y} is selected from the groups depicted in the compounds in Table 1.

As defined generally above, each instance of R^{CyA} is independently -L^{CyA}-R^{CyAA}.

In some embodiments, each instance of R^{CyA} is independently —C(O)N(H)—R^{CyAA}, —C(O)N(H)CH₂—R^{CyAA}, or —R^{CyAA}. In some embodiments, each instance of R^{CyA} is independently —C(O)N(H)—R^{CyAA}. In some embodiments, each instance of R^{CyA} is independently —C(O)N(H)CH₂—R^{CyAA}. In some embodiments, each instance of R^{CyA} is independently —R^{CyAA}.

In some embodiments each instance of R^{CyA} is independently

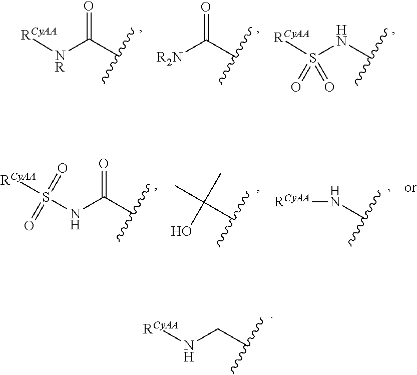

In some embodiments, each instance of R^{CyA} is independently

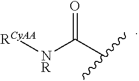

In some embodiments, each instance of R^{CyA} is independently

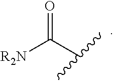

In some embodiments, each instance of R^{CyA} is independently

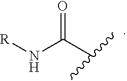

In some embodiments, each instance of R^{CyA} is independently

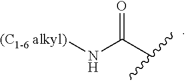

In some embodiments, each instance of R^{CyA} is

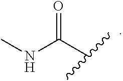 [SMILES: *C(=O)NC]

In some embodiments, each instance of R^{CyA} is independently

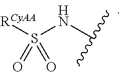

In some embodiments, each instance of R^{CyA} is independently

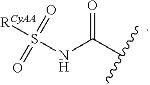

In some embodiments, each instance of each instance of R^{CyA} is

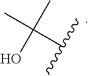 [SMILES: *C(C)(C)O]

In some embodiments, each instance of R^{CyA} is independently

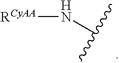

In some embodiments, each instance of R^{CyA} is independently

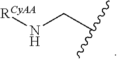

In some embodiments, each instance of R^{CyA} is independently R^{B} substituted by r⁶ instances of R^{CyAC}. In some embodiments, each instance of R^{CyA} is independently a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; wherein each ring is substituted by r⁶ instances of R^{CyAC}. In some embodiments, each instance of R^{CyA} is independently a 5-6 membered monocyclic heteroaryl ring having 1-4 heteroatoms independently selected from nitrogen, oxygen, and sulfur; wherein said ring is substituted by r⁶ instances of R^{CyAC}. In some embodiments, each instance of R^{CyA} is independently a 5-6 membered monocyclic heteroaryl ring having 1-2 nitrogen atoms; wherein said ring is substituted by r⁶ instances of R^{CyAC}. In some embodiments, each instance of R^{CyA} is independently a 3-7 membered saturated or partially unsaturated monocyclic heterocyclic ring having 1-2 heteroatoms independently selected from nitrogen, oxygen, and sulfur; wherein said ring is substituted by r⁶ instances of R^{CyAC}.

In some embodiments, each instance of R^{CyA} is independently

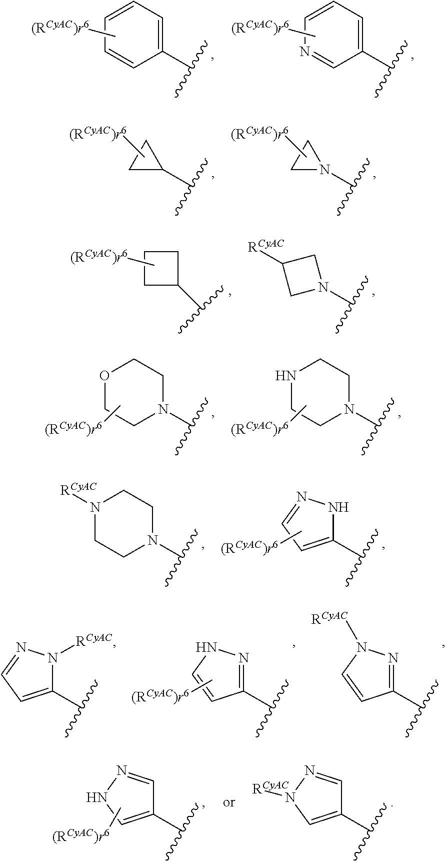

In some embodiments, each instance of R^{CyA} is independently

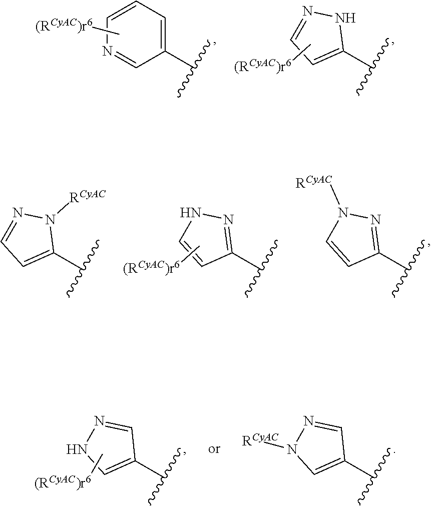

In some embodiments, each instance of R^{CyA} is independently

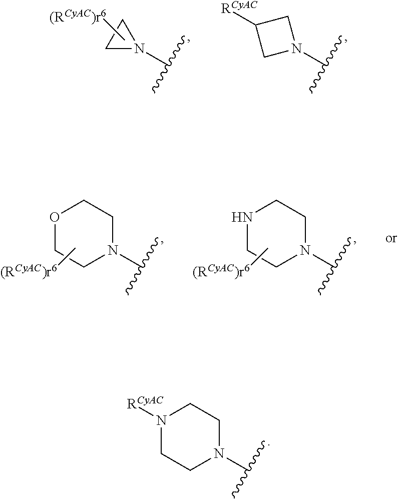

In some embodiments, each instance of R^{CyA} is independently

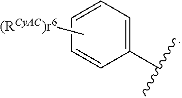

In some embodiments, each instance of R^{CyA} is independently

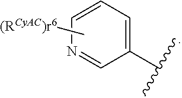

In some embodiments, each instance of R^{CyA} is independently

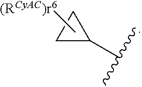

In some embodiments, each instance of R^{CyA} is independently

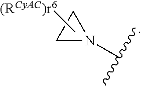

In some embodiments, each instance of R^{CyA} is independently

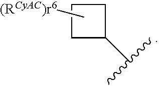

In some embodiments, each instance of R^{CyA} is independently

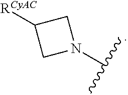

In some embodiments, each instance of R^{CyA} is independently

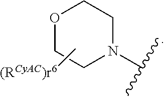

In some embodiments, each instance of R^{CyA} is independently

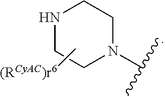

In some embodiments, each instance of R^{CyA} is independently

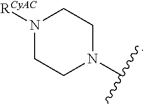

In some embodiments, each instance of R^{CyA} is independently

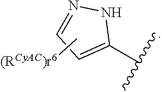

In some embodiments, each instance of R^{CyA} is independently

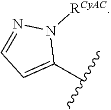

In some embodiments, each instance of R^{CyA} is independently

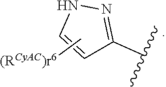

In some embodiments, each instance of R^{CyA} is independently

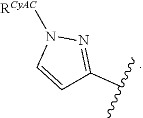

In some embodiments, each instance of R^{CyA} is independently

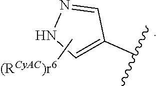

In some embodiments, each instance of R^{CyA} is independently

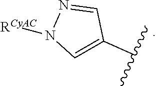

In some embodiments, each instance of R^{CyA} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, each instance of R^{CyA} is independently a C₁₋₆ aliphatic that is (i) substituted with 1 or 2 groups independently selected from —O—(C₁₋₆ aliphatic), —OH, —N(C₁₋₆ aliphatic)₂, and —CN, and (ii) optionally substituted with 1, 2, or 3 atoms independently selected from halogen and deuterium. In some embodiments, each instance of R^{CyA} is independently a C₁₋₆ aliphatic optionally substituted with 1 or 2 groups independently selected from —O—(C₁₋₆ aliphatic), —OH, —N(C₁₋₆ aliphatic)₂, and —CN. In some embodiments, each instance of R^{CyA} is independently a C₁₋₆ aliphatic substituted with 1 or 2 groups independently selected from —O—(C₁₋₆ aliphatic), —OH, —N(C₁₋₆ aliphatic)₂, and —CN.

In some embodiments, each instance of R^{CyA} is independently a C₁₋₆ aliphatic optionally substituted with 1, 2, or 3 atoms independently selected from halogen and deuterium. In some embodiments, each instance of R^{CyA} is independently a C₁₋₆ aliphatic substituted with 1, 2, or 3 atoms independently selected from halogen and deuterium. In some embodiments, each instance of R^{CyA} is independently a C₁₋₆ aliphatic.

In some embodiments, each instance of R^{CyA} is independently selected from the groups depicted in the compounds in Table 1.

As defined generally above, Cy^{A} is a 5-6 membered saturated or partially unsaturated monocyclic ring having 0-3 heteroatoms independently selected from nitrogen, oxygen, and sulfur; or a 8-10 membered saturated, partially unsaturated, or aromatic bicyclic ring having 0-4 heteroatoms independently selected from nitrogen, oxygen, and sulfur; wherein each ring is substituted with n instances of R^{CyA}.

In some embodiments, Cy^{A} is a 5-6 membered saturated or partially unsaturated monocyclic ring having 0-3 heteroatoms independently selected from nitrogen, oxygen, and sulfur, wherein the monocyclic ring is substituted with n instances of R^{CyA}. In some embodiments, Cy^{A} is a 5-membered saturated or partially unsaturated monocyclic ring having 0-3 heteroatoms independently selected from nitrogen, oxygen, and sulfur, wherein the monocyclic ring is substituted with n instances of R^{CyA}. In some embodiments, Cy^{A} is a 6-membered saturated or partially unsaturated monocyclic ring having 0-3 heteroatoms independently selected from nitrogen, oxygen, and sulfur, wherein the monocyclic ring is substituted with n instances of R^{CyA}.

In some embodiments, Cy^{A} is a 8-10 membered saturated, partially unsaturated, or aromatic bicyclic ring having 0-4 heteroatoms independently selected from nitrogen, oxygen, and sulfur, wherein each ring is substituted with n instances of R^{CyA}. In some embodiments, Cy^{A} is a 8-membered saturated, partially unsaturated, or aromatic bicyclic ring having 0-4 heteroatoms independently selected from nitrogen, oxygen, and sulfur, wherein each ring is substituted with n instances of R^{CyA}. In some embodiments, Cy^{A} is a 9-membered saturated, partially unsaturated, or aromatic bicyclic ring having 0-4 heteroatoms independently selected from nitrogen, oxygen, and sulfur, wherein each ring is substituted with n instances of R^{CyA}. In some embodiments, Cy^{A} is a 10-membered saturated, partially unsaturated, or aromatic bicyclic ring having 0-4 heteroatoms independently selected from nitrogen, oxygen, and sulfur, wherein each ring is substituted with n instances of R^{CyA}.

In some embodiments, Cy^{A} is a monocyclic or bicyclic ring selected from cyclopentane, cyclohexane, pyrrolidine, pyrazole, thiophene, piperidine, piperazine, benzene, pyridine, pyridazine, pyrimidine, pyrazine, indoline, 1H-indole, [1,2,4]triazolo[4,3-a]pyridine, and quinoline; wherein each ring is substituted with n instances of R^{CyA}.

In some embodiments, Cy^{A} is cyclopentane substituted with n instances of R^{CyA}. In some embodiments, Cy^{A} is cyclohexane substituted with n instances of R^{CyA}. In some embodiments, Cy^{A} is pyrrolidine substituted with n instances of R^{CyA}. In some embodiments, Cy^{A} is pyrazole substituted with n instances of R^{CyA}. In some embodiments, Cy^{A} is thiophene substituted with n instances of R^{CyA}. In some embodiments, Cy^{A} is piperidine substituted with n instances of R^{CyA}. In some embodiments, Cy^{A} is piperazine substituted with n instances of R^{CyA}. In some embodiments, Cy^{A} is benzene substituted with n instances of R^{CyA}. In some embodiments, Cy^{A} is pyridine substituted with n instances of R^{CyA}. In some embodiments, Cy^{A} is pyridazine substituted with n instances of R^{CyA}. In some embodiments, Cy^{A} is pyrimidine substituted with n instances of R^{CyA}. In some embodiments, Cy^{A} is pyrazine substituted with n instances of R^{CyA}. In some embodiments, Cy^{A} is indoline substituted with n instances of R^{CyA}. In some embodiments, Cy^{A} is 1H-indole substituted with n instances of R^{CyA}. In some embodiments, Cy^{A} is [1,2,4]triazolo[4,3-a]pyridine substituted with n instances of R^{CyA}. In some embodiments, Cy^{A} is quinoline substituted with n instances of R^{CyA}.

In some embodiments, Cy^{A} is

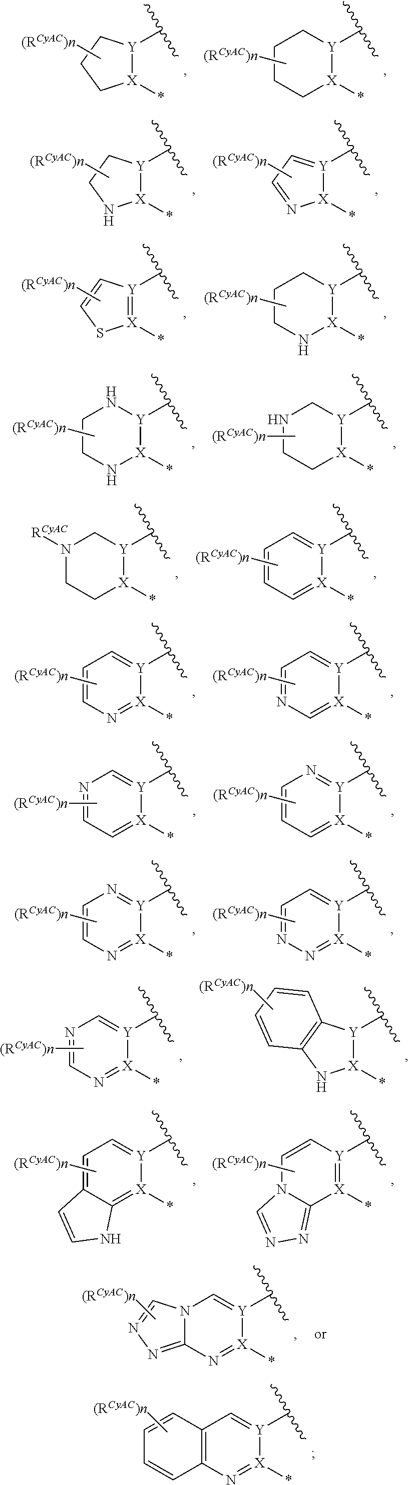

wherein [symbol] represents a bond to R¹ and [symbol] represents a bond to R².

In some embodiments, Cy^{A} is

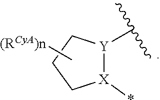

In some embodiments, Cy^{A} is

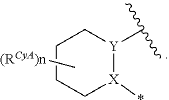

In some embodiments, Cy^{A} is

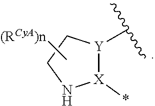

In some embodiments, Cy^{A} is

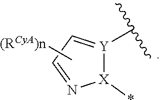

In some embodiments, Cy^{A} is

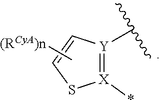

In some embodiments, Cy^{A} is

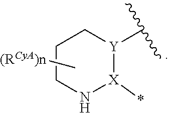

In some embodiments, Cy^{A} is

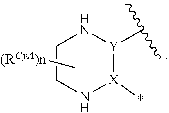

In some embodiments, Cy^{A} is

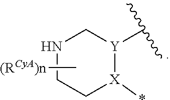

In some embodiments, Cy^{A} is

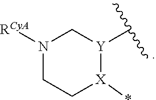

In some embodiments, Cy^{A} is

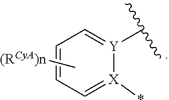

In some embodiments, Cy^{A} is

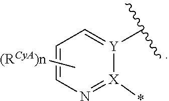

In some embodiments, Cy^{A} is

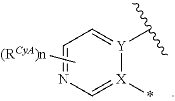

In some embodiments, Cy^{A} is

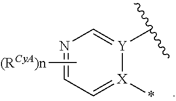

In some embodiments, Cy^{A} is

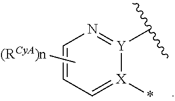

In some embodiments, Cy^{A} is

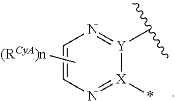

In some embodiments, Cy^{A} is

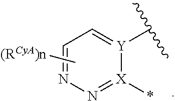

In some embodiments, Cy^{A} is

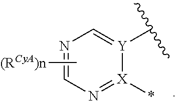

In some embodiments, Cy^{A} is

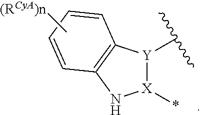

In some embodiments, Cy^{A} is

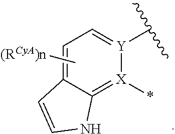

In some embodiments, Cy^{A} is

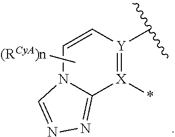

In some embodiments, Cy^{A} is

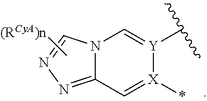

In some embodiments, Cy^{A} is

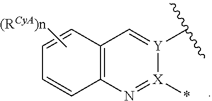

In some embodiments, Cy^{A} is selected from the groups depicted in the compounds in Table 1.

As defined generally above, L¹ is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a covalent bond. In some embodiments, L¹ is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₄ bivalent saturated or unsaturated, straight or branched hydrocarbon chain.

In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L¹ is a C₁₋₂ bivalent saturated or unsaturated hydrocarbon chain.

In some embodiments, L¹ is —N(H)—, —CH₂—, or a covalent bond. In some embodiments, L¹ is is —N(H)—. In some embodiments, L¹ is —CH₂—. In some embodiments, L¹ is selected from the groups depicted in the compounds in Table 1.

As defined generally above, L² is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a covalent bond. In some embodiments, L² is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₄ bivalent saturated or unsaturated, straight or branched hydrocarbon chain.

In some embodiments, L² is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L² is a C₁₋₂ bivalent saturated or unsaturated hydrocarbon chain.

In some embodiments, L² is —N(R)C(O)—, —N(R)C(O)N(R)—, —C(O)N(R)—, —N(R)—, —S(O)₂CH₂—, —CH₂S(O)₂—, or a covalent bond. In some embodiments, L² is —N(H)C(O)—, —N(H)C(O)N(H)—, —C(O)N(H)—, —N(H)—, —S(O)₂CH₂—, —CH₂S(O)₂—, or a covalent bond. In some embodiments, L² is —N(R)C(O)—, —N(R)C(O)N(R)—, —N(R)—, or a covalent bond. In some embodiments, L² is —N(H)C(O)—, —N(H)C(O)N(H)—, —N(H)—, or a covalent bond.

In some embodiments, L² is —N(R)C(O)— or —N(R)C(O)N(R)—. In some embodiments, L² is —N(H)C(O)— or —N(H)C(O)N(H)—. In some embodiments, L² is —N(R)C(O)—. In some embodiments, L² is —N(H)C(O)—. In some embodiments, L² is —N(R)C(O)N(R)—. In some embodiments, L² is —N(H)C(O)N(H)—. In some embodiments, L² is —C(O)N(R)—. In some embodiments, L² is —C(O)N(H)—. In some embodiments, L² is —N(R)—. In some embodiments, L² is —N(H)—. In some embodiments, L² is —S(O)₂CH₂— or —CH₂S(O)₂—. In some embodiments, L² is —S(O)₂CH₂—. In some embodiments, L² is —CH₂S(O)₂—. In some embodiments, L² is a covalent bond. In some embodiments, L² is selected from the groups depicted in the compounds in Table 1.

As defined generally above, L^{X} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a covalent bond. In some embodiments, L^{X} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a C₁₋₄ bivalent saturated or unsaturated, straight or branched hydrocarbon chain.

In some embodiments, L^{X} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{X} is a C₁₋₂ bivalent saturated or unsaturated hydrocarbon chain. In some embodiments, L^{X} is selected from the groups depicted in the compounds in Table 1.

As defined generally above, L^{Y} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y} is a covalent bond. In some embodiments, L^{Y} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y} is a C₁₋₄ bivalent saturated or unsaturated, straight or branched hydrocarbon chain.

In some embodiments, L^{Y} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{Y} is a C₁₋₂ bivalent saturated or unsaturated hydrocarbon chain.

In some embodiments, L^{Y} is —C(O)N(R)—, —C(O)N(R)CH₂—, or a covalent bond. In some embodiments, L^{Y} is —C(O)N(H)—, —C(O)N(H)CH₂—, or a covalent bond. In some embodiments, L^{Y} is —C(O)N(H)— or —C(O)N(H)CH₂—. In some embodiments, L^{Y} is —C(O)N(H)—. In some embodiments, L^{Y} is —C(O)N(H)CH₂—. In some embodiments, L^{Y} is selected from the groups depicted in the compounds in Table 1.

As defined generally above, L^{CyA} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{CyA} is a covalent bond. In some embodiments, L^{CyA} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{CyA} is a C₁₋₄ bivalent saturated or unsaturated, straight or branched hydrocarbon chain.

In some embodiments, L^{CyA} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{CyA} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{CyA} is a C₁₋₂ bivalent saturated or unsaturated hydrocarbon chain.

In some embodiments, L^{CyA} is —C(O)N(R)—, —C(O)N(R)CH₂—, or a covalent bond. In some embodiments, L^{CyA} is —C(O)N(H)—, —C(O)N(H)CH₂—, or a covalent bond. In some embodiments, L^{CyA} is —C(O)N(H)— or —C(O)N(H)CH₂—. In some embodiments, L^{CyA} is —C(O)N(H)—. In some embodiments, L^{CyA} is —C(O)N(H)CH₂—. In some embodiments, L^{CyA} is selected from the groups depicted in the compounds in Table 1.

As defined generally above, R^{1A} is R^{A} or R^{B} substituted by r¹ instances of R^{1C}. In some embodiments, R^{1A} is R^{A}. In some embodiments, R^{1A} is R^{B} substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{1A} is substituted by r¹ instances of R^{1C}.

In some embodiments, R^{1A} is phenyl substituted by r¹ instances of R^{1C}. In some embodiments, R^{1A} is an 8-10 membered bicyclic heteroaryl ring having 1-4 heteroatoms independently selected from nitrogen, oxygen, and sulfur, wherein R^{1A} is substituted by r¹ instances of R^{1C}. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; or a 3-7 membered saturated or partially unsaturated monocyclic carbocyclic ring; wherein R^{1A} is substituted by r¹ instances of R^{1C}.

In some embodiments, R^{1A} is phenyl substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{1A} is phenyl substituted by 1-3 instances of R^{1C}. In some embodiments, R^{1A} is phenyl substituted by 2 instances of R^{1C}. In some embodiments, R^{1A} is phenyl substituted by 1 instance of R^{1C}.

In some embodiments, R^{1A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{1A} is phenyl substituted by 1-3 instances of a group independently selected from halogen and C₁₋₃ aliphatic optionally substituted with 1-3 halogen. In some embodiments, R^{1A} is phenyl substituted by 1-3 instances of a group independently selected from fluorine, chlorine, —CH₃, —CHF₂, and —CF₃.

In some embodiments, R^{1A} is phenyl substituted by 2 instances of a group independently selected from halogen, —CN, —O-(optionally substituted C₁₋₆ aliphatic), and an optionally substituted C₁₋₆ aliphatic. In some embodiments, R^{1A} is phenyl substituted by 2 instances of a group independently selected from halogen and C₁₋₃ aliphatic optionally substituted with 1-3 halogen. In some embodiments, R^{1A} is phenyl substituted by 2 instances of a group independently selected from fluorine, chlorine, —CH₃, —CHF₂, and —CF₃.

In some embodiments, R^{1A} is phenyl substituted by one group selected from halogen, —CN, —O-(optionally substituted C₁₋₆ aliphatic), and an optionally substituted C₁₋₆ aliphatic. In some embodiments, R^{1A} is phenyl substituted by one halogen or C₁₋₃ aliphatic group optionally substituted with 1-3 halogen. In some embodiments, R^{1A} is phenyl substituted by one fluorine, chlorine, —CH₃, —CHF₂, or —CF₃.

In some embodiments, R^{1A} is

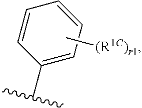

wherein R^{1C} and r¹ are as defined in the embodiments and classes and subclasses herein. In some embodiments, R^{1A} is

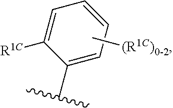

wherein R^{1C} is as defined in the embodiments and classes and subclasses herein. In some embodiments, R^{1A} is

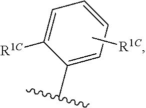

wherein R^{1C} is as defined in the embodiments and classes and subclasses herein. In some embodiments, R^{1A} is

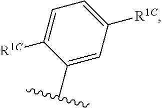

wherein R^{1C} is as defined in the embodiments and classes and subclasses herein. In some embodiments, R^{1A} is

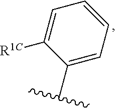

wherein R^{1C} is as defined in the embodiments and classes and subclasses herein.

In some embodiments, R^{1A} is

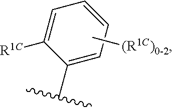

wherein each instance of R^{1C} is independently halogen, —CN, —O-(optionally substituted C₁₋₆ aliphatic), or an optionally substituted C₁₋₆ aliphatic. In some embodiments, R^{1A} is

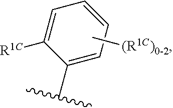

wherein each instance of R^{1C} is independently halogen or C₁₋₃ aliphatic optionally substituted with 1-3 halogen. In some embodiments, R^{1A} is

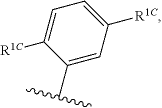

wherein each instance of R^{1C} is independently halogen or C₁₋₃ aliphatic optionally substituted with 1-3 halogen. In some embodiments, R^{1A} is

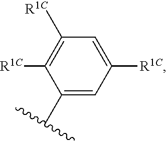

wherein each instance of R^{1C} is independently halogen or C₁₋₃ aliphatic optionally substituted with 1-3 halogen. In some embodiments, R^{1A} is

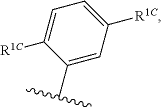

wherein each instance of R^{1C} is independently fluorine, chlorine, —CH₃, —CHF₂, or —CF₃. In some embodiments, R^{1A} is

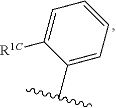

wherein R^{1C} is halogen or C₁₋₃ aliphatic optionally substituted with 1-3 halogen.

In some embodiments, R^{1A} is

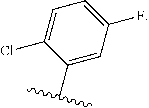 [SMILES: *c1cc(F)ccc1Cl]

In some embodiments, R^{1A} is

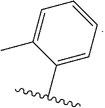 [SMILES: *c1ccccc1C]

In some embodiments, R^{1A} is

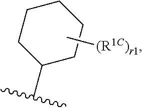

wherein R^{1C} and r¹ are as defined in the embodiments and classes and subclasses herein. In some embodiments, R^{1A} is

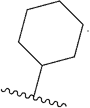 [SMILES: *C1CCCCC1]

In some embodiments, R^{1A} is

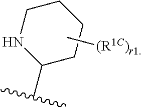

In some embodiments, R^{1A} is

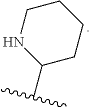 [SMILES: *C1CCCCN1]

In some embodiments, R^{1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{1A} is oxo. In some embodiments, R^{1A} is halogen. In some embodiments, R^{1A} is —CN. In some embodiments, R^{1A} is —NO₂. In some embodiments, R^{1A} is —OR. In some embodiments, R^{1A} is —SR. In some embodiments, R^{1A} is —NR₂. In some embodiments, R^{1A} is —S(O)₂R. In some embodiments, R^{1A} is —S(O)₂NR₂. In some embodiments, R^{1A} is —S(O)₂F. In some embodiments, R^{1A} is —S(O)R. In some embodiments, R^{1A} is —S(O)NR₂. In some embodiments, R^{1A} is —S(O)(NR)R. In some embodiments, R^{1A} is —C(O)R. In some embodiments, R^{1A} is —C(O)OR. In some embodiments, R^{1A} is —C(O)NR₂. In some embodiments, R^{1A} is —C(O)N(R)OR. In some embodiments, R^{1A} is —OC(O)R. In some embodiments, R^{1A} is —OC(O)NR₂. In some embodiments, R^{1A} is —N(R)C(O)OR. In some embodiments, R^{1A} is —N(R)C(O)R. In some embodiments, R^{1A} is —N(R)C(O)NR₂. In some embodiments, R^{1A} is —N(R)C(NR)NR₂. In some embodiments, R^{1A} is —N(R)S(O)₂NR₂.

In some embodiments, R^{1A} is —N(R)S(O)₂R. In some embodiments, R^{1A} is —P(O)R₂. In some embodiments, R^{1A} is —P(O)(R)OR. In some embodiments, R^{1A} is —B(OR)₂. In some embodiments, R^{1A} is deuterium.

In some embodiments, R^{1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{1A} is halogen, —CN, or —NO₂. In some embodiments, R^{1A} is —OR, —SR, or —NR₂. In some embodiments, R^{1A} is —S(O)₂R, —S(O)₂NR₂, —S(O)₂F, —S(O)R, —S(O)NR₂, or —S(O)(NR)R. In some embodiments, R^{1A} is —C(O)R, —C(O)OR, —C(O)NR₂, or —C(O)N(R)OR. In some embodiments, R^{1A} is —OC(O)R or —OC(O)NR₂. In some embodiments, R^{1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{1A} is —P(O)R₂ or —P(O)(R)OR.

In some embodiments, R^{1A} is —OR, —OC(O)R, or —OC(O)NR₂. In some embodiments, R^{1A} is —SR, —S(O)₂R, —S(O)₂NR₂, —S(O)₂F, —S(O)R, —S(O)NR₂, or —S(O)(NR)R. In some embodiments, R^{1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{1A} is —S(O)₂R, —S(O)₂NR₂, or —S(O)₂F. In some embodiments, R^{1A} is —S(O)R, —S(O)NR₂, or —S(O)(NR)R. In some embodiments, R^{1A} is —SR, —S(O)₂R, or —S(O)R. In some embodiments, R^{1A} is —S(O)₂NR₂, —S(O)NR₂, or —S(O)(NR)R. In some embodiments, R^{1A} is —S(O)₂NR₂ or —S(O)NR₂. In some embodiments, R^{1A} is —SR, —S(O)₂R, —S(O)₂NR₂, or —S(O)R.

In some embodiments, R^{1A} is —N(R)C(O)OR, —N(R)C(O)R, or —N(R)C(O)NR₂. In some embodiments, R^{1A} is —N(R)S(O)₂NR₂ or —N(R)S(O)₂R. In some embodiments, R^{1A} is —N(R)C(O)OR or —N(R)C(O)R. In some embodiments, R^{1A} is —N(R)C(O)NR₂ or —N(R)S(O)₂NR₂. In some embodiments, R^{1A} is —N(R)C(O)OR, —N(R)C(O)R, or —N(R)S(O)₂R.

In some embodiments, R^{1A} is —NR₂, —N(R)C(O)OR, —N(R)C(O)R, or —N(R)C(O)NR₂. In some embodiments, R^{1A} is —NR₂, —N(R)C(O)OR, or —N(R)C(O)R. In some embodiments, R^{1A} is —NR₂, —N(R)C(O)OR, —N(R)C(O)R, or —N(R)S(O)₂R.

In some embodiments, R^{1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain substituted by r¹ instances of R^{1C}. In some embodiments, R^{1A} is phenyl substituted by r¹ instances of R^{1C}. In some embodiments, R^{1A} is naphthyl substituted by r¹ instances of R^{1C}. In some embodiments, R^{1A} is a 5-6 membered monocyclic heteroaryl ring having 1-4 heteroatoms independently selected from nitrogen, oxygen, and sulfur; wherein said ring is substituted by r¹ instances of R^{1C}. In some embodiments, R^{1A} is an 8-10 membered bicyclic heteroaryl ring having 1-4 heteroatoms independently selected from nitrogen, oxygen, and sulfur; wherein said ring is substituted by r¹ instances of R^{1C}. In some embodiments, R^{1A} is a 3-7 membered saturated or partially unsaturated monocyclic carbocyclic ring substituted by r¹ instances of R^{1C}. In some embodiments, R^{1A} is a 5-12 membered saturated or partially unsaturated bicyclic carbocyclic ring substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; wherein said ring is substituted by r¹ instances of R^{1C}. In some embodiments, R^{1A} is a 7-12 membered saturated or partially unsaturated bicyclic heterocyclic ring having 1-4 heteroatoms independently selected from nitrogen, oxygen, and sulfur; wherein said ring is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or naphthyl; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 5-12 membered saturated or partially unsaturated bicyclic carbocyclic ring; each of which is substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 5-6 membered monocyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 3-7 membered saturated or partially unsaturated monocyclic carbocyclic ring; each of which is substituted by r¹ instances of R^{1C}. In some embodiments, R^{1A} is naphthyl or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}. In some embodiments, R^{1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}.

In some embodiments, R^{1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r¹ instances of R^{1C}. In some embodiments, R^{1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is a C₁₋₆ aliphatic chain, phenyl, or a 3-7 membered saturated or partially unsaturated monocyclic carbocyclic ring; each of which is substituted by r¹ instances of R^{1C}.

In some embodiments, R^{1A} is selected from the groups depicted in the compounds in Table 1.

As defined generally above, R^{2A} is R^{A} or R^{B} substituted by r² instances of R^{2C}. In some embodiments, R^{2A} is R^{A}. In some embodiments, R^{2A} is R^{B} substituted by r² instances of R^{2C}.

In some embodiments, R^{2A} is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)O R, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by r² instances of R^{2C}. In some embodiments, R^{2A} is phenyl substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{2A} is phenyl substituted by 1-3 instances of a group independently selected from halogen and C₁₋₃ aliphatic optionally substituted with 1-3 halogen. In some embodiments, R^{2A} is phenyl substituted by 1-3 instances of a group independently selected from fluorine, chlorine, —CH₃, —CHF₂, and —CF₃.

In some embodiments, R^{2A} is phenyl substituted by 2 instances of a group independently selected from halogen, —CN, —O-(optionally substituted C₁₋₆ aliphatic), and an optionally substituted C₁₋₆ aliphatic. In some embodiments, R^{2A} is phenyl substituted by 2 instances of a group independently selected from halogen and C₁₋₃ aliphatic optionally substituted with 1-3 halogen. In some embodiments, R^{2A} is phenyl substituted by 2 instances of a group independently selected from fluorine, chlorine, —CH₃, —CHF₂, and —CF₃.

In some embodiments, R^{2A} is an 8-10 membered bicyclic heteroaryl ring having 1-4 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-2 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen, —CN, —O-(optionally substituted C₁₋₆ aliphatic), and an optionally substituted C₁₋₆ aliphatic. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen and C₁₋₃ aliphatic optionally substituted with 1-3 halogen. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from fluorine, chlorine, —CH₃, —CHF₂, and —CF₃.

In some embodiments, R^{2A} is:

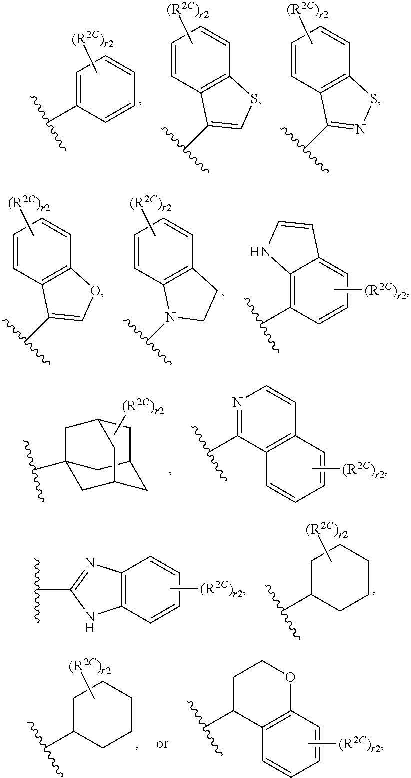

wherein R^{2C} and r² are as defined in the embodiments and classes and subclasses herein. In some embodiments, R^{2A} is

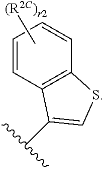

In some embodiments, R^{2A} is

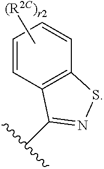

In some embodiments, R^{2A} is

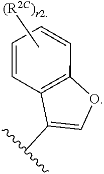

In some embodiments, R^{2A} is

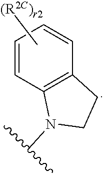

In some embodiments, R^{2A} is

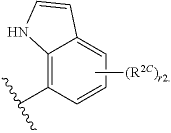

In some embodiments, R^{2A} is

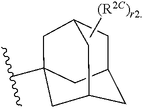

In some embodiments, R^{2A} is

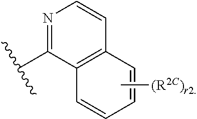

In some embodiments, R^{2A} is

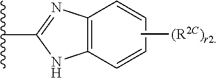

In some embodiments, R^{2A} is

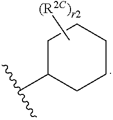

In some embodiments, R^{2A} is

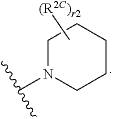

In some embodiments, R^{2A} is

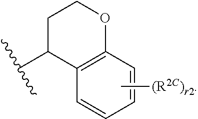

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{2A} is oxo. In some embodiments, R^{2A} is halogen. In some embodiments, R^{2A} is —CN. In some embodiments, R^{2A} is —NO₂. In some embodiments, R^{2A} is —OR. In some embodiments, R^{2A} is —SR. In some embodiments, R^{2A} is —NR₂. In some embodiments, R^{2A} is —S(O)₂R. In some embodiments, R^{2A} is —S(O)₂NR₂. In some embodiments, R^{2A} is —S(O)₂F. In some embodiments, R^{2A} is —S(O)R. In some embodiments, R^{2A} is —S(O)NR₂. In some embodiments, R^{2A} is —S(O)(NR)R. In some embodiments, R^{2A} is —C(O)R. In some embodiments, R^{2A} is —C(O)OR. In some embodiments, R^{2A} is —C(O)NR₂. In some embodiments, R^{2A} is —C(O)N(R)OR. In some embodiments, R^{2A} is —OC(O)R. In some embodiments, R^{2A} is —OC(O)NR₂. In some embodiments, R^{2A} is —N(R)C(O)OR. In some embodiments, R^{2A} is —N(R)C(O)R. In some embodiments, R^{2A} is —N(R)C(O)NR₂. In some embodiments, R^{2A} is —N(R)C(NR)NR₂. In some embodiments, R^{2A} is —N(R)S(O)₂NR₂. In some embodiments, R^{2A} is —N(R)S(O)₂R. In some embodiments, R^{2A} is —P(O)R₂. In some embodiments, R^{2A} is —P(O)(R)OR. In some embodiments, R^{2A} is —B(OR)₂. In some embodiments, R^{2A} is deuterium.

In some embodiments, R^{2A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{2A} is halogen, —CN, or —NO₂. In some embodiments, R^{2A} is —OR, —SR, or —NR₂. In some embodiments, R^{2A} is —S(O)₂R, —S(O)₂NR₂, —S(O)₂F, —S(O)R, —S(O)NR₂, or —S(O)(NR)R. In some embodiments, R^{2A} is —C(O)R, —C(O)OR, —C(O)NR₂, or —C(O)N(R)OR. In some embodiments, R^{2A} is —OC(O)R or —OC(O)NR₂. In some embodiments, R^{2A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{2A} is —P(O)R₂ or —P(O)(R)OR.

In some embodiments, R^{2A} is —OR, —OC(O)R, or —OC(O)NR₂. In some embodiments, R^{2A} is —SR, —S(O)₂R, —S(O)₂NR₂, —S(O)₂F, —S(O)R, —S(O)NR₂, or —S(O)(NR)R. In some embodiments, R^{2A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{2A} is —S(O)₂R, —S(O)₂NR₂, or —S(O)₂F. In some embodiments, R^{2A} is —S(O)R, —S(O)NR₂, or —S(O)(NR)R. In some embodiments, R^{2A} is —SR, —S(O)₂R, or —S(O)R. In some embodiments, R^{2A} is —S(O)₂NR₂, —S(O)NR₂, or —S(O)(NR)R. In some embodiments, R^{2A} is —S(O)₂NR₂ or —S(O)NR₂. In some embodiments, R^{2A} is —SR, —S(O)₂R, —S(O)₂NR₂, or —S(O)R.

In some embodiments, R^{2A} is —N(R)C(O)OR, —N(R)C(O)R, or —N(R)C(O)NR₂. In some embodiments, R^{2A} is —N(R)S(O)₂NR₂ or —N(R)S(O)₂R. In some embodiments, R^{2A} is —N(R)C(O)OR or —N(R)C(O)R. In some embodiments, R^{2A} is —N(R)C(O)NR₂ or —N(R)S(O)₂NR₂. In some embodiments, R^{2A} is —N(R)C(O)OR, —N(R)C(O)R, or —N(R)S(O)₂R.

In some embodiments, R^{2A} is —NR₂, —N(R)C(O)OR, —N(R)C(O)R, or —N(R)C(O)NR₂. In some embodiments, R^{2A} is —NR₂, —N(R)C(O)OR, or —N(R)C(O)R. In some embodiments, R^{2A} is —NR₂, —N(R)C(O)OR, —N(R)C(O)R, or —N(R)S(O)₂R.

In some embodiments, R^{2A} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain substituted by r² instances of R^{2C} In some embodiments, R^{2A} is phenyl substituted by r² instances of R^{2C}. In some embodiments, R^{2A} is naphthyl substituted by r² instances of R^{2C}. In some embodiments, R^{2A} is cubanyl substituted by r² instances of R^{2C}. In some embodiments, R^{2A} is adamantyl substituted by r² instances of R^{2C}. In some embodiments, R^{2A} is a 5-6 membered monocyclic heteroaryl ring having 1-4 heteroatoms independently selected from nitrogen, oxygen, and sulfur; wherein said ring is substituted by r² instances of R^{2C}. In some embodiments, R^{2A} is an 8-10 membered bicyclic heteroaryl ring having 1-4 heteroatoms independently selected from nitrogen, oxygen, and sulfur; wherein said ring is substituted by r² instances of R^{2C}. In some embodiments, R^{2A} is a 3-7 membered saturated or partially unsaturated monocyclic carbocyclic ring substituted by r² instances of R^{2C}. In some embodiments, R^{2A} is a 5-12 membered saturated or partially unsaturated bicyclic carbocyclic ring substituted by r² instances of R^{2C}. In some embodiments, R^{2A} is a 3-7 membered saturated or partially unsaturated monocyclic heterocyclic ring having 1-2 heteroatoms independently selected from nitrogen, oxygen, and sulfur; wherein said ring is substituted by r² instances of R^{2C}. In some embodiments, R^{2A} is a 7-12 membered saturated or partially unsaturated bicyclic heterocyclic ring having 1-4 heteroatoms independently selected from nitrogen, oxygen, and sulfur; wherein said ring is substituted by r² instances of R^{2C}.

In some embodiments, R^{2A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl; cubanyl; adamantyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or naphthyl; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 5-12 membered saturated or partially unsaturated bicyclic carbocyclic ring; each of which is substituted by r² instances of R^{2C}. In some embodiments, R^{2A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 5-6 membered monocyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 3-7 membered saturated or partially unsaturated monocyclic carbocyclic ring; each of which is substituted by r² instances of R^{2C}. In some embodiments, R^{2A} is naphthyl; cubanyl; adamantyl; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain; cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}. In some embodiments, R^{2A} is a C₁₋₆ aliphatic chain; phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}.

In some embodiments, R^{2A} is a C₁₋₆ aliphatic chain, cubanyl, adamantyl, a 3-7 membered saturated or partially unsaturated monocyclic carbocyclic ring, or a 5-12 membered saturated or partially unsaturated bicyclic carbocyclic ring; each of which is substituted by r² instances of R^{2C}. In some embodiments, R^{2A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is a C₁₋₆ aliphatic chain, phenyl, or a 3-7 membered saturated or partially unsaturated monocyclic carbocyclic ring; each of which is substituted by r² instances of R^{2C}.

In some embodiments, R^{2A} is selected from the groups depicted in the compounds in Table 1.

As defined generally above, R^{XA} is R^{A} or R^{B} substituted by r³ instances of R^{XC}. In some embodiments, R^{XA} is R^{A}. In some embodiments, R^{XA} is R^{B} substituted by r³ instances of R^{XC}.

In some embodiments, R^{XA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{XA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{XA} is oxo. In some embodiments, R^{XA} is halogen. In some embodiments, R^{XA} is —CN. In some embodiments, R^{XA} is —NO₂. In some embodiments, R^{XA} is —OR. In some embodiments, R^{XA} is —SR. In some embodiments, R^{XA} is —NR₂. In some embodiments, R^{XA} is —S(O)₂R. In some embodiments, R^{XA} is —S(O)₂NR₂. In some embodiments, R^{XA} is —S(O)₂F. In some embodiments, R^{XA} is —S(O)R. In some embodiments, R^{XA} is —S(O)NR₂. In some embodiments, R^{XA} is —S(O)(NR)R. In some embodiments, R^{XA} is —C(O)R. In some embodiments, R^{XA} is —C(O)OR. In some embodiments, R^{XA} is —C(O)NR₂. In some embodiments, R^{XA} is —C(O)N(R)OR. In some embodiments, R^{XA} is —OC(O)R. In some embodiments, R^{XA} is —OC(O)NR₂. In some embodiments, R^{XA} is —N(R)C(O)OR. In some embodiments, R^{XA} is —N(R)C(O)R. In some embodiments, R^{XA} is —N(R)C(O)NR₂. In some embodiments, R^{XA} is —N(R)C(NR)NR₂. In some embodiments, R^{XA} is —N(R)S(O)₂NR₂. In some embodiments, R^{XA} is —N(R)S(O)₂R. In some embodiments, R^{XA} is —P(O)R₂. In some embodiments, R^{XA} is —P(O)(R)OR. In some embodiments, R^{XA} is —B(OR)₂. In some embodiments, R^{XA} is deuterium.

In some embodiments, R^{XA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{XA} is halogen, —CN, or —NO₂. In some embodiments, R^{XA} is —OR, —SR, or —NR₂. In some embodiments, R^{XA} is —S(O)₂R, —S(O)₂NR₂, —S(O)₂F, —S(O)R, —S(O)NR₂, or —S(O)(NR)R. In some embodiments, R^{XA} is —C(O)R, —C(O)OR, —C(O)NR₂, or —C(O)N(R)OR. In some embodiments, R^{XA} is —OC(O)R or —OC(O)NR₂. In some embodiments, R^{XA} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{XA} is —P(O)R₂ or —P(O)(R)OR.

In some embodiments, R^{XA} is —OR, —OC(O)R, or —OC(O)NR₂. In some embodiments, R^{XA} is —SR, —S(O)₂R, —S(O)₂NR₂, —S(O)₂F, —S(O)R, —S(O)NR₂, or —S(O)(NR)R. In some embodiments, R^{XA} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{XA} is —S(O)₂R, —S(O)₂NR₂, or —S(O)₂F. In some embodiments, R^{XA} is —S(O)R, —S(O)NR₂, or —S(O)(NR)R. In some embodiments, R^{XA} is —SR, —S(O)₂R, or —S(O)R. In some embodiments, R^{XA} is —S(O)₂NR₂, —S(O)NR₂, or —S(O)(NR)R. In some embodiments, R^{XA} is —S(O)₂NR₂ or —S(O)NR₂. In some embodiments, R^{XA} is —SR, —S(O)₂R, —S(O)₂NR₂, or —S(O)R.

In some embodiments, R^{XA} is —N(R)C(O)OR, —N(R)C(O)R, or —N(R)C(O)NR₂. In some embodiments, R^{XA} is —N(R)S(O)₂NR₂ or —N(R)S(O)₂R. In some embodiments, R^{XA} is —N(R)C(O)OR or —N(R)C(O)R. In some embodiments, R^{XA} is —N(R)C(O)NR₂ or —N(R)S(O)₂NR₂. In some embodiments, R^{XA} is —N(R)C(O)OR, —N(R)C(O)R, or —N(R)S(O)₂R.

In some embodiments, R^{XA} is —NR₂, —N(R)C(O)OR, —N(R)C(O)R, or —N(R)C(O)NR₂. In some embodiments, R^{XA} is —NR₂, —N(R)C(O)OR, or —N(R)C(O)R. In some embodiments, R^{XA} is —NR₂, —N(R)C(O)OR, —N(R)C(O)R, or —N(R)S(O)₂R.

In some embodiments, R^{XA} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is a C₁₋₆ aliphatic chain substituted by r³ instances of R^{XC}. In some embodiments, R^{XA} is phenyl substituted by r³ instances of R^{XC}. In some embodiments, R^{XA} is naphthyl substituted by r³ instances of R^{XC}. In some embodiments, R^{XA} is a 5-6 membered monocyclic heteroaryl ring having 1-4 heteroatoms independently selected from nitrogen, oxygen, and sulfur; wherein said ring is substituted by r³ instances of R^{XC}. In some embodiments, R^{XA} is an 8-10 membered bicyclic heteroaryl ring having 1-4 heteroatoms independently selected from nitrogen, oxygen, and sulfur; wherein said ring is substituted by r³ instances of R^{XC}. In some embodiments, R^{XA} is a 3-7 membered saturated or partially unsaturated monocyclic carbocyclic ring substituted by r³ instances of R^{XC}. In some embodiments, R^{XA} is a 5-12 membered saturated or partially unsaturated bicyclic carbocyclic ring substituted by r³ instances of R^{XC}. In some embodiments, R^{XA} is a 3-7 membered saturated or partially unsaturated monocyclic heterocyclic ring having 1-2 heteroatoms independently selected from nitrogen, oxygen, and sulfur; wherein said ring is substituted by r³ instances of R^{XC}. In some embodiments, R^{XA} is a 7-12 membered saturated or partially unsaturated bicyclic heterocyclic ring having 1-4 heteroatoms independently selected from nitrogen, oxygen, and sulfur; wherein said ring is substituted by r³ instances of R^{XC}.

In some embodiments, R^{XA} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r³ instances of R^{XC}. In some embodiments, R^{XA} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl; naphthyl; a 3-7 membered saturated or partially unsaturated monocyclic carbocyclic ring; or a 5-12 membered saturated or partially unsaturated bicyclic carbocyclic ring; each of which is substituted by r³ instances of R^{XC}. In some embodiments, R^{XA} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r³ instances of R^{XC}. In some embodiments, R^{XA} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl or naphthyl; each of which is substituted by r³ instances of R^{XC}. In some embodiments, R^{XA} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r³ instances of R^{XC}. In some embodiments, R^{XA} is a 3-7 membered saturated or partially unsaturated monocyclic carbocyclic ring or a 5-12 membered saturated or partially unsaturated bicyclic carbocyclic ring; each of which is substituted by r³ instances of R^{XC}. In some embodiments, R^{XA} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl or a 5-6 membered monocyclic heteroaryl ring having 1-4 heteroatoms independently selected from nitrogen, oxygen, and sulfur; each of which is substituted by r³ instances of R^{XC}. In some embodiments, R^{XA} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r³ instances of R^{XC}. In some embodiments, R^{XA} is naphthyl or an 8-10 membered bicyclic heteroaryl ring having 1-4 heteroatoms independently selected from nitrogen, oxygen, and sulfur; each of which is substituted by r³ instances of R^{XC}. In some embodiments, R^{XA} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl or a 3-7 membered saturated or partially unsaturated monocyclic carbocyclic ring; each of which is substituted by r³ instances of R^{XC} In some embodiments, R^{XA} is naphthyl or a 5-12 membered saturated or partially unsaturated bicyclic carbocyclic ring; each of which is substituted by r³ instances of R^{XC}. In some embodiments, R^{XA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r³ instances of R^{XC}. In some embodiments, R^{XA} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}. In some embodiments, R^{XA} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r³ instances of R^{XC}. In some embodiments, R^{XA} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r³ instances of R^{XC}.

In some embodiments, R^{XA} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r³ instances of R^{XC}. In some embodiments, R^{XA} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r³ instances of R^{XC}. In some embodiments, R^{XA} is a C₁₋₆ aliphatic chain, phenyl, or a 3-7 membered saturated or partially unsaturated monocyclic carbocyclic ring; each of which is substituted by r³ instances of R^{XC}.

In some embodiments, R^{XA} is selected from the groups depicted in the compounds in Table 1.

As defined generally above, R^{YA} is R^{A} or R^{B} substituted by r⁴ instances of R^{YC}. In some embodiments, R^{YA} is R^{A}. In some embodiments, R^{YA} is R^{B} substituted by r⁴ instances of R^{YC}.

In some embodiments, R^{YA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{YA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{YA} is oxo. In some embodiments, R^{YA} is halogen. In some embodiments, R^{YA} is —CN. In some embodiments, R^{YA} is —NO₂. In some embodiments, R^{YA} is —OR. In some embodiments, R^{YA} is —SR. In some embodiments, R^{YA} is —NR₂. In some embodiments, R^{YA} is —S(O)₂R. In some embodiments, R^{YA} is —S(O)₂NR₂. In some embodiments, R^{YA} is —S(O)₂F. In some embodiments, R^{YA} is —S(O)R. In some embodiments, R^{YA} is —S(O)NR₂. In some embodiments, R^{YA} is —S(O)(NR)R. In some embodiments, R^{YA} is —C(O)R. In some embodiments, R^{YA} is —C(O)OR. In some embodiments, R^{YA} is —C(O)NR₂. In some embodiments, R^{YA} is —C(O)N(R)OR. In some embodiments, R^{YA} is —OC(O)R. In some embodiments, R^{YA} is —OC(O)NR₂. In some embodiments, R^{YA} is —N(R)C(O)OR. In some embodiments, R^{YA} is —N(R)C(O)R. In some embodiments, R^{YA} is —N(R)C(O)NR₂. In some embodiments, R^{YA} is —N(R)C(NR)NR₂. In some embodiments, R^{YA} is —N(R)S(O)₂NR₂. In some embodiments, R^{YA} is —N(R)S(O)₂R. In some embodiments, R^{YA} is —P(O)R₂. In some embodiments, R^{YA} is —P(O)(R)OR. In some embodiments, R^{YA} is —B(OR)₂. In some embodiments, R^{YA} is deuterium.

In some embodiments, R^{YA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{YA} is halogen, —CN, or —NO₂. In some embodiments, R^{YA} is —OR, —SR, or —NR₂. In some embodiments, R^{YA} is —S(O)₂R, —S(O)₂NR₂, —S(O)₂F, —S(O)R, —S(O)NR₂, or —S(O)(NR)R. In some embodiments, R^{YA} is —C(O)R, —C(O)OR, —C(O)NR₂, or —C(O)N(R)OR. In some embodiments, R^{YA} is —OC(O)R or —OC(O)NR₂. In some embodiments, R^{YA} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{YA} is —P(O)R₂ or —P(O)(R)OR.

In some embodiments, R^{YA} is —OR, —OC(O)R, or —OC(O)NR₂. In some embodiments, R^{YA} is —SR, —S(O)₂R, —S(O)₂NR₂, —S(O)₂F, —S(O)R, —S(O)NR₂, or —S(O)(NR)R. In some embodiments, R^{YA} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{YA} is —S(O)₂R, —S(O)₂NR₂, or —S(O)₂F. In some embodiments, R^{YA} is —S(O)R, —S(O)NR₂, or —S(O)(NR)R. In some embodiments, R^{YA} is —SR, —S(O)₂R, or —S(O)R. In some embodiments, R^{YA} is —S(O)₂NR₂, —S(O)NR₂, or —S(O)(NR)R. In some embodiments, R^{YA} is —S(O)₂NR₂ or —S(O)NR₂. In some embodiments, R^{YA} is —SR, —S(O)₂R, —S(O)₂NR₂, or —S(O)R.

In some embodiments, R^{YA} is —N(R)C(O)OR, —N(R)C(O)R, or —N(R)C(O)NR₂. In some embodiments, R^{YA} is —N(R)S(O)₂NR₂ or —N(R)S(O)₂R. In some embodiments, R^{Y}A is —N(R)C(O)OR or —N(R)C(O)R. In some embodiments, R^{YA} is —N(R)C(O)NR₂ or —N(R)S(O)₂NR₂. In some embodiments, R^{YA} is —N(R)C(O)OR, —N(R)C(O)R, or —N(R)S(O)₂R.

In some embodiments, R^{YA} is —NR₂, —N(R)C(O)OR, —N(R)C(O)R, or —N(R)C(O)NR₂. In some embodiments, R^{YA} is —NR₂, —N(R)C(O)OR, or —N(R)C(O)R. In some embodiments, R^{YA} is —NR₂, —N(R)C(O)OR, —N(R)C(O)R, or —N(R)S(O)₂R.

In some embodiments, R^{YA} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is a C₁₋₆ aliphatic chain substituted by r⁴ instances of R^{YC}. In some embodiments, R^{YA} is phenyl substituted by r⁴ instances of R^{YC}. In some embodiments, R^{YA} is naphthyl substituted by r⁴ instances of R^{YC}. In some embodiments, R^{YA} is a 5-6 membered monocyclic heteroaryl ring having 1-4 heteroatoms independently selected from nitrogen, oxygen, and sulfur; wherein said ring is substituted by r⁴ instances of R^{YC}. In some embodiments, R^{YA} is an 8-10 membered bicyclic heteroaryl ring having 1-4 heteroatoms independently selected from nitrogen, oxygen, and sulfur; wherein said ring is substituted by r⁴ instances of R^{YC}. In some embodiments, R^{YA} is a 3-7 membered saturated or partially unsaturated monocyclic carbocyclic ring substituted by r⁴ instances of R^{YC}. In some embodiments, R^{YA} is a 5-12 membered saturated or partially unsaturated bicyclic carbocyclic ring substituted by r⁴ instances of R^{YC}. In some embodiments, R^{YA} is a 3-7 membered saturated or partially unsaturated monocyclic heterocyclic ring having 1-2 heteroatoms independently selected from nitrogen, oxygen, and sulfur; wherein said ring is substituted by r⁴ instances of R^{YC}. In some embodiments, R^{YA} is a 7-12 membered saturated or partially unsaturated bicyclic heterocyclic ring having 1-4 heteroatoms independently selected from nitrogen, oxygen, and sulfur; wherein said ring is substituted by r⁴ instances of R^{YC}.

In some embodiments, R^{YA} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁴ instances of R^{YC}. In some embodiments, R^{YA} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl; naphthyl; a 3-7 membered saturated or partially unsaturated monocyclic carbocyclic ring; or a 5-12 membered saturated or partially unsaturated bicyclic carbocyclic ring; each of which is substituted by r⁴ instances of R^{YC}. In some embodiments, R^{YA} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁴ instances of R^{YC}. In some embodiments, R^{YA} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl or naphthyl; each of which is substituted by r⁴ instances of R^{YC}. In some embodiments, R^{YA} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁴ instances of R^{YC}. In some embodiments, R^{YA} is a 3-7 membered saturated or partially unsaturated monocyclic carbocyclic ring or a 5-12 membered saturated or partially unsaturated bicyclic carbocyclic ring; each of which is substituted by r⁴ instances of R^{YC}. In some embodiments, R^{YA} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl or a 5-6 membered monocyclic heteroaryl ring having 1-4 heteroatoms independently selected from nitrogen, oxygen, and sulfur; each of which is substituted by r⁴ instances of R^{YC}. In some embodiments, R^{YA} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁴ instances of R^{YC}. In some embodiments, R^{YA} is naphthyl or an 8-10 membered bicyclic heteroaryl ring having 1-4 heteroatoms independently selected from nitrogen, oxygen, and sulfur; each of which is substituted by r⁴ instances of R^{YC}. In some embodiments, R^{YA} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl or a 3-7 membered saturated or partially unsaturated monocyclic carbocyclic ring; each of which is substituted by r⁴ instances of R^{YC}. In some embodiments, R^{YA} is naphthyl or a 5-12 membered saturated or partially unsaturated bicyclic carbocyclic ring; each of which is substituted by r⁴ instances of R^{YC}. In some embodiments, R^{YA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁴ instances of R^{YC}. In some embodiments, R^{YA} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}. In some embodiments, R^{YA} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁴ instances of R^{YC}. In some embodiments, R^{YA} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁴ instances of R^{YC}.

In some embodiments, R^{YA} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁴ instances of R^{YC}. In some embodiments, R^{YA} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁴ instances of R^{YC}. In some embodiments, R^{YA} is a C₁₋₆ aliphatic chain, phenyl, or a 3-7 membered saturated or partially unsaturated monocyclic carbocyclic ring; each of which is substituted by r⁴ instances of R^{YC}.

In some embodiments, R^{YA} is selected from the groups depicted in the compounds in Table 1.

As defined generally above, R^{L} is R^{A} or R^{B} substituted by r⁵ instances of R^{LC}. In some embodiments, R^{L} is R^{A}. In some embodiments, R^{L} is R^{B} substituted by r⁵ instances of R^{LC}.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L} is oxo. In some embodiments, R^{L} is halogen. In some embodiments, R^{L} is —CN. In some embodiments, R^{L} is —NO₂. In some embodiments, R^{L} is —OR. In some embodiments, R^{L} is —SR. In some embodiments, R^{L} is —NR₂. In some embodiments, R^{L} is —S(O)₂R. In some embodiments, R^{L} is —S(O)₂NR₂. In some embodiments, R^{L} is —S(O)₂F. In some embodiments, R^{L} is —S(O)R. In some embodiments, R^{L} is —S(O)NR₂. In some embodiments, R^{L} is —S(O)(NR)R. In some embodiments, R^{L} is —C(O)R. In some embodiments, R^{L} is —C(O)OR. In some embodiments, R^{L} is —C(O)NR₂. In some embodiments, R^{L} is —C(O)N(R)OR. In some embodiments, R^{L} is —OC(O)R. In some embodiments, R^{L} is —OC(O)NR₂. In some embodiments, R^{L} is —N(R)C(O)OR. In some embodiments, R^{L} is —N(R)C(O)R. In some embodiments, R^{L} is —N(R)C(O)NR₂. In some embodiments, R^{L} is —N(R)C(NR)NR₂. In some embodiments, R^{L} is —N(R)S(O)₂NR₂. In some embodiments, R^{L} is —N(R)S(O)₂R. In some embodiments, R^{L} is —P(O)R₂. In some embodiments, R^{L} is —P(O)(R)OR. In some embodiments, R^{L} is —B(OR)₂. In some embodiments, R^{L} is deuterium.

In some embodiments, R^{L} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L} is halogen, —CN, or —NO₂. In some embodiments, R^{L} is —OR, —SR, or —NR₂. In some embodiments, R^{L} is —S(O)₂R, —S(O)₂NR₂, —S(O)₂F, —S(O)R, —S(O)NR₂, or —S(O)(NR)R. In some embodiments, R^{L} is —C(O)R, —C(O)OR, —C(O)NR₂, or —C(O)N(R)OR. In some embodiments, R^{L} is —OC(O)R or —OC(O)NR₂. In some embodiments, R^{L} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{L} is —P(O)R₂ or —P(O)(R)OR.

In some embodiments, R^{L} is —OR, —OC(O)R, or —OC(O)NR₂. In some embodiments, R^{L} is —SR, —S(O)₂R, —S(O)₂NR₂, —S(O)₂F, —S(O)R, —S(O)NR₂, or —S(O)(NR)R. In some embodiments, R^{L} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{L} is —S(O)₂R, —S(O)₂NR₂, or —S(O)₂F. In some embodiments, R^{L} is —S(O)R, —S(O)NR₂, or —S(O)(NR)R. In some embodiments, R^{L} is —SR, —S(O)₂R, or —S(O)R. In some embodiments, R^{L} is —S(O)₂NR₂, —S(O)NR₂, or —S(O)(NR)R. In some embodiments, R^{L} is —S(O)₂NR₂ or —S(O)NR₂. In some embodiments, R^{L} is —SR, —S(O)₂R, —S(O)₂NR₂, or —S(O)R.

In some embodiments, R^{L} is —N(R)C(O)OR, —N(R)C(O)R, or —N(R)C(O)NR₂. In some embodiments, R^{L} is —N(R)S(O)₂NR₂ or —N(R)S(O)₂R. In some embodiments, R^{L} is —N(R)C(O)OR or —N(R)C(O)R. In some embodiments, R^{L} is —N(R)C(O)NR₂ or —N(R)S(O)₂NR₂. In some embodiments, R^{L} is —N(R)C(O)OR, —N(R)C(O)R, or —N(R)S(O)₂R.

In some embodiments, R^{L} is —NR₂, —N(R)C(O)OR, —N(R)C(O)R, or —N(R)C(O)NR₂. In some embodiments, R^{L} is —NR₂, —N(R)C(O)OR, or —N(R)C(O)R. In some embodiments, R^{L} is —NR₂, —N(R)C(O)OR, —N(R)C(O)R, or —N(R)S(O)₂R.

In some embodiments, R^{L} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is a C₁₋₆ aliphatic chain substituted by r⁵ instances of R^{LC}. In some embodiments, R^{L} is phenyl substituted by r⁵ instances of R^{LC}. In some embodiments, R^{L} is naphthyl substituted by r⁵ instances of R^{LC}. In some embodiments, R^{L} is a 5-6 membered monocyclic heteroaryl ring having 1-4 heteroatoms independently selected from nitrogen, oxygen, and sulfur; wherein said ring is substituted by r⁵ instances of R^{LC}. In some embodiments, R^{L} is an 8-10 membered bicyclic heteroaryl ring having 1-4 heteroatoms independently selected from nitrogen, oxygen, and sulfur; wherein said ring is substituted by r⁵ instances of R^{LC}. In some embodiments, R^{L} is a 3-7 membered saturated or partially unsaturated monocyclic carbocyclic ring substituted by r⁵ instances of R^{LC}. In some embodiments, R^{L} is a 5-12 membered saturated or partially unsaturated bicyclic carbocyclic ring substituted by r⁵ instances of R^{LC}. In some embodiments, R^{L} is a 3-7 membered saturated or partially unsaturated monocyclic heterocyclic ring having 1-2 heteroatoms independently selected from nitrogen, oxygen, and sulfur; wherein said ring is substituted by r⁵ instances of R^{LC}. In some embodiments, R^{L} is a 7-12 membered saturated or partially unsaturated bicyclic heterocyclic ring having 1-4 heteroatoms independently selected from nitrogen, oxygen, and sulfur; wherein said ring is substituted by r⁵ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁵ instances of R^{LC}. In some embodiments, R^{L} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl; naphthyl; a 3-7 membered saturated or partially unsaturated monocyclic carbocyclic ring; or a 5-12 membered saturated or partially unsaturated bicyclic carbocyclic ring; each of which is substituted by r⁵ instances of R^{LC}. In some embodiments, R^{L} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁵ instances of R^{LC}. In some embodiments, R^{L} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl or naphthyl; each of which is substituted by r⁵ instances of R^{LC}. In some embodiments, R^{L} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁵ instances of R^{LC}. In some embodiments, R^{L} is a 3-7 membered saturated or partially unsaturated monocyclic carbocyclic ring or a 5-12 membered saturated or partially unsaturated bicyclic carbocyclic ring; each of which is substituted by r⁵ instances of R^{LC}. In some embodiments, R^{L} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl or a 5-6 membered monocyclic heteroaryl ring having 1-4 heteroatoms independently selected from nitrogen, oxygen, and sulfur; each of which is substituted by r⁵ instances of R^{LC}. In some embodiments, R^{L} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁵ instances of R^{LC}. In some embodiments, R^{L} is naphthyl or an 8-10 membered bicyclic heteroaryl ring having 1-4 heteroatoms independently selected from nitrogen, oxygen, and sulfur; each of which is substituted by r⁵ instances of R^{LC}. In some embodiments, R^{L} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl or a 3-7 membered saturated or partially unsaturated monocyclic carbocyclic ring; each of which is substituted by r⁵ instances of R^{LC}. In some embodiments, R^{L} is naphthyl or a 5-12 membered saturated or partially unsaturated bicyclic carbocyclic ring; each of which is substituted by r⁵ instances of R^{LC}. In some embodiments, R^{L} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁵ instances of R^{LC}. In some embodiments, R^{L} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}. In some embodiments, R^{L} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁵ instances of R^{LC}. In some embodiments, R^{L} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁵ instances of R^{LC}.

In some embodiments, R^{L} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁵ instances of R^{LC}. In some embodiments, R^{L} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁵ instances of R^{LC}. In some embodiments, R^{L} is a C₁₋₆ aliphatic chain, phenyl, or a 3-7 membered saturated or partially unsaturated monocyclic carbocyclic ring; each of which is substituted by r⁵ instances of R^{LC}.

In some embodiments, R^{L} is selected from the groups depicted in the compounds in Table 1.

As generally defined above, each instance of R^{CyAA} is independently R^{A} or R^{B} substituted by r⁶ instances of R^{CyAC}. In some embodiments, each instance of R^{CyAA} is independently R^{A}. In some embodiments, each instance of R^{CyAA} is independently R^{B} substituted by r⁶ instances of R^{CyAC}.

In some embodiments, each instance of R^{CyAA} is independently a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; wherein each ring is substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; wherein said ring is substituted by r⁶ instances of R^{CyAC}. In some embodiments, each instance of R^{CyAA} is independently a 5-6 membered monocyclic heteroaryl ring having 1-2 nitrogen atoms; wherein said ring is substituted by r⁶ instances of R^{CyAC}. In some embodiments, each instance of R^{CyAA} is independently a 3-7 membered saturated or partially unsaturated monocyclic heterocyclic ring having 1-2 heteroatoms independently selected from nitrogen, oxygen, and sulfur; wherein said ring is substituted by r⁶ instances of R^{CyAC}.

In some embodiments, each instance of R^{CyAA} is independently

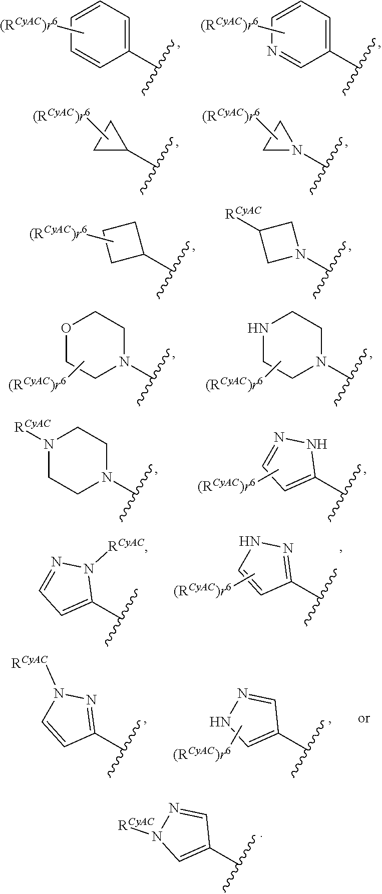

In some embodiments, each instance of R^{CyAA} is independently

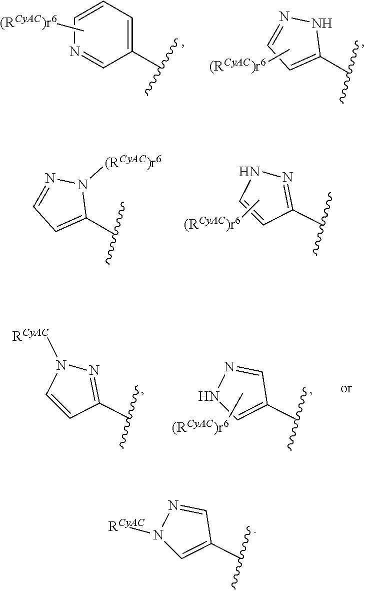

In some embodiments, each instance of R^{CyAA} is independently

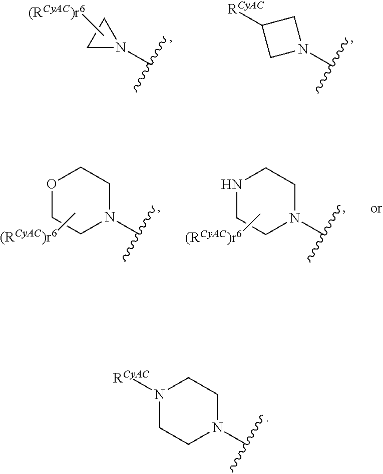

In some embodiments, each instance of R^{CyAA} is independently

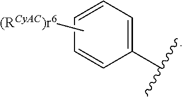

In some embodiments, each instance of R^{CyAA} is independently

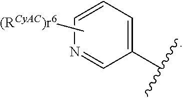

In some embodiments, each instance of R^{CyAA} is independently

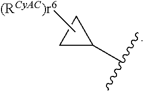

In some embodiments, each instance of R^{CyAA} is independently

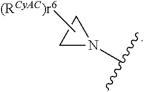

In some embodiments, each instance of R^{CyAA} is independently

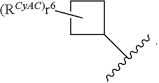

In some embodiments, each instance of R^{CyAA} is independently

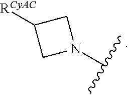

In some embodiments, each instance of R^{CyAA} is independently

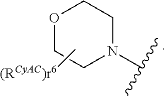

In some embodiments, each instance of R^{CyAA} is independently

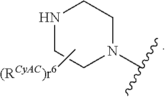

In some embodiments, each instance of R^{CyAA} is independently

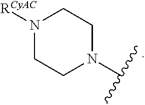

In some embodiments, each instance of R^{CyAA} is independently

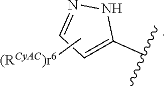

In some embodiments, each instance of R^{CyAA} is independently

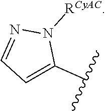

In some embodiments, each instance of R^{CyAA} is independently

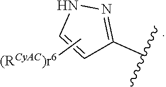

In some embodiments, each instance of R^{CyAA} is independently

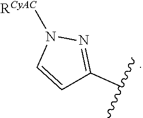

In some embodiments, each instance of R^{CyAA} is independently

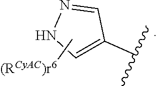

In some embodiments, each instance of R^{CyAA} is independently

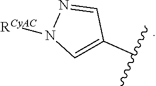

In some embodiments, each instance of R^{CyAA} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, each instance of R^{CyAA} is independently a C₁₋₆ aliphatic that is (i) substituted with 1 or 2 groups independently selected from —O—(C₁₋₆ aliphatic), —OH, —N(C₁₋₆ aliphatic)₂, and —CN, and (ii) optionally substituted with 1, 2, or 3 atoms independently selected from halogen and deuterium. In some embodiments, each instance of R^{CyAA} is independently a C₁₋₆ aliphatic optionally substituted with 1 or 2 groups independently selected from —O—(C₁₋₆ aliphatic), —OH, —N(C₁₋₆ aliphatic)₂, and —CN. In some embodiments, each instance of R^{CyAA} is independently a C₁₋₆ aliphatic substituted with 1 or 2 groups independently selected from —O—(C₁₋₆ aliphatic), —OH, —N(C₁₋₆ aliphatic)₂, and —CN.

In some embodiments, each instance of R^{CyAA} is independently a C₁₋₆ aliphatic optionally substituted with 1, 2, or 3 atoms independently selected from halogen and deuterium. In some embodiments, each instance of R^{CyAA} is independently a C₁₋₆ aliphatic substituted with 1, 2, or 3 atoms independently selected from halogen and deuterium. In some embodiments, each instance of R^{CyAA} is independently a C₁₋₆ aliphatic.

In some embodiments, each instance of R^{CyAA} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, each instance of R^{CyAA} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{CyAA} is oxo. In some embodiments, each instance of R^{CyAA} is independently halogen. In some embodiments, each instance of R^{CyAA} is —CN. In some embodiments, each instance of R^{CyAA} is —NO₂. In some embodiments, each instance of R^{CyAA} is independently —OR. In some embodiments, each instance of R^{CyAA} is independently —SR. In some embodiments, each instance of R^{CyAA} is independently —NR₂. In some embodiments, each instance of R^{CyAA} is independently —S(O)₂R. In some embodiments, each instance of R^{CyAA} is independently —S(O)₂NR₂. In some embodiments, each instance of R^{CyAA} is —S(O)₂F. In some embodiments, each instance of R^{CyAA} is independently —S(O)R. In some embodiments, each instance of R^{CyAA} is independently —S(O)NR₂. In some embodiments, each instance of R^{CyAA} is independently —S(O)(NR)R. In some embodiments, each instance of R^{CyAA} is independently —C(O)R. In some embodiments, each instance of R^{CyAA} is independently —C(O)OR. In some embodiments, each instance of R^{CyAA} is independently —C(O)NR₂. In some embodiments, each instance of R^{CyAA} is independently —C(O)N(R)OR. In some embodiments, each instance of R^{CyAA} is independently —OC(O)R. In some embodiments, each instance of R^{CyAA} is independently —OC(O)NR₂. In some embodiments, each instance of R^{CyAA} is independently —N(R)C(O)OR. In some embodiments, each instance of R^{CyAA} is independently —N(R)C(O)R. In some embodiments, each instance of R^{CyAA} is independently —N(R)C(O)NR₂. In some embodiments, each instance of R^{CyAA} is independently —N(R)C(NR)NR₂. In some embodiments, each instance of R^{CyAA} is independently —N(R)S(O)₂NR₂. In some embodiments, each instance of R^{CyAA} is independently —N(R)S(O)₂R. In some embodiments, each instance of R^{CyAA} is independently —P(O)R₂. In some embodiments, each instance of R^{CyAA} is independently —P(O)(R)OR. In some embodiments, each instance of R^{CyAA} is independently —B(OR)₂. In some embodiments, each instance of R^{CyAA} is deuterium.

In some embodiments, each instance of R^{CyAA} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{CyAA} is independently halogen, —CN, or —NO₂. In some embodiments, each instance of R^{CyAA} is independently —OR, —SR, or —NR₂. In some embodiments, each instance of R^{CyAA} is independently —S(O)₂R, —S(O)₂NR₂, —S(O)₂F, —S(O)R, —S(O)NR₂, or —S(O)(NR)R. In some embodiments, each instance of R^{CyAA} is independently —C(O)R, —C(O)OR, —C(O)NR₂, or —C(O)N(R)OR. In some embodiments, each instance of R^{CyAA} is independently —OC(O)R or —OC(O)NR₂. In some embodiments, each instance of R^{CyAA} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{CyAA} is independently —P(O)R₂ or —P(O)(R)OR.

In some embodiments, each instance of R^{CyAA} is independently —OR, —OC(O)R, or —OC(O)NR₂. In some embodiments, each instance of R^{CyAA} is independently —SR, —S(O)₂R, —S(O)₂NR₂, —S(O)₂F, —S(O)R, —S(O)NR₂, or —S(O)(NR)R. In some embodiments, each instance of R^{CyAA} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{CyAA} is independently —S(O)₂R, —S(O)₂NR₂, or —S(O)₂F. In some embodiments, each instance of R^{CyAA} is independently —S(O)R, —S(O)NR₂, or —S(O)(NR)R. In some embodiments, each instance of R^{CyAA} is independently —SR, —S(O)₂R, or —S(O)R. In some embodiments, each instance of R^{CyAA} is independently —S(O)₂NR₂, —S(O)NR₂, or —S(O)(NR)R. In some embodiments, each instance of R^{CyAA} is independently —S(O)₂NR₂ or —S(O)NR₂. In some embodiments, each instance of R^{CyAA} is independently —SR, —S(O)₂R, —S(O)₂NR₂, or —S(O)R.

In some embodiments, each instance of R^{CyAA} is independently —N(R)C(O)OR, —N(R)C(O)R, or —N(R)C(O)NR₂. In some embodiments, each instance of R^{CyAA} is independently —N(R)S(O)₂NR₂ or —N(R)S(O)₂R. In some embodiments, each instance of R^{CyAA} is independently —N(R)C(O)OR or —N(R)C(O)R. In some embodiments, each instance of R^{CyAA} is independently —N(R)C(O)NR₂ or —N(R)S(O)₂NR₂. In some embodiments, each instance of R^{CyAA} is independently —N(R)C(O)OR, —N(R)C(O)R, or —N(R)S(O)₂R.

In some embodiments, each instance of R^{CyAA} is independently —NR₂, —N(R)C(O)OR, —N(R)C(O)R, or —N(R)C(O)NR₂. In some embodiments, each instance of R^{CyAA} is independently —NR₂, —N(R)C(O)OR, or —N(R)C(O)R. In some embodiments, each instance of R^{CyAA} is independently —NR₂, —N(R)C(O)OR, —N(R)C(O)R, or —N(R)S(O)₂R.

In some embodiments, each instance of R^{CyAA} is independently a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently a C₁₋₆ aliphatic chain substituted by r⁶ instances of R^{CyAC}. In some embodiments, each instance of R^{CyAA} is independently phenyl substituted by r⁶ instances of R^{CyAC}. In some embodiments, each instance of R^{CyAA} is independently naphthyl substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; wherein said ring is substituted by r⁶ instances of R^{CyAC}. In some embodiments, each instance of R^{CyAA} is independently an 8-10 membered bicyclic heteroaryl ring having 1-4 heteroatoms independently selected from nitrogen, oxygen, and sulfur; wherein said ring is substituted by r⁶ instances of R^{CyAC}. In some embodiments, each instance of R^{CyAA} is independently a 3-7 membered saturated or partially unsaturated monocyclic carbocyclic ring substituted by r⁶ instances of R^{CyAC}. In some embodiments, each instance of R^{Cy} is independently a 5-12 membered saturated or partially unsaturated bicyclic carbocyclic ring substituted by r⁶ instances of R^{CyAC}. In some embodiments, each instance of R^{CyAA} is independently a 3-7 membered saturated or partially unsaturated monocyclic heterocyclic ring having 1-2 heteroatoms independently selected from nitrogen, oxygen, and sulfur; wherein said ring is substituted by r⁶ instances of R^{CyAC}. In some embodiments, each instance of R^{CyAA} is independently a 7-12 membered saturated or partially unsaturated bicyclic heterocyclic ring having 1-4 heteroatoms independently selected from nitrogen, oxygen, and sulfur; wherein said ring is substituted by r⁶ instances of R^{CyAC}.

In some embodiments, each instance of R^{CyAA} is independently phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁶ instances of R^{CyAC}. In some embodiments, each instance of R^{CyAA} is independently a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl; naphthyl; a 3-7 membered saturated or partially unsaturated monocyclic carbocyclic ring; or a 5-12 membered saturated or partially unsaturated bicyclic carbocyclic ring; each of which is substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}. In some embodiments, each instance of R^{CyAA} is independently naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl or naphthyl; each of which is substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁶ instances of R^{CyAC}. In some embodiments, each instance of R^{CyAA} is independently a 3-7 membered saturated or partially unsaturated monocyclic carbocyclic ring or a 5-12 membered saturated or partially unsaturated bicyclic carbocyclic ring; each of which is substituted by r⁶ instances of R^{CyAC}. In some embodiments, each instance of R^{CyAA} is independently a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl or a 5-6 membered monocyclic heteroaryl ring having 1-4 heteroatoms independently selected from nitrogen, oxygen, and sulfur; each of which is substituted by r⁶ instances of R^{CyAC}. In some embodiments, each instance of R^{CyAA} is independently a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}. In some embodiments, each instance of R^{CyAA} is independently naphthyl or an 8-10 membered bicyclic heteroaryl ring having 1-4 heteroatoms independently selected from nitrogen, oxygen, and sulfur; each of which is substituted by r⁶ instances of R^{CyAC}. In some embodiments, each instance of R^{CyAA} is independently a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl or a 3-7 membered saturated or partially unsaturated monocyclic carbocyclic ring; each of which is substituted by r⁶ instances of R^{CyAC}. In some embodiments, each instance of R^{CyAA} is independently naphthyl or a 5-12 membered saturated or partially unsaturated bicyclic carbocyclic ring; each of which is substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}. In some embodiments, each instance of R^{CyAA} is independently an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}. In some embodiments, each instance of R^{CyAA} is independently a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁶ instances of R^{CyAC}. In some embodiments, each instance of R^{CyAA} is independently a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}.

In some embodiments, each instance of R^{CyAA} is independently a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁶ instances of R^{CyAC}. In some embodiments, each instance of R^{CyAA} is independently a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}. In some embodiments, each instance of R^{CyAA} is independently a C₁₋₆ aliphatic chain, phenyl, or a 3-7 membered saturated or partially unsaturated monocyclic carbocyclic ring; each of which is substituted by r⁶ instances of R^{CyAC}.

In some embodiments, each instance of R^{CyAA} is independently selected from the groups depicted in the compounds in Table 1.

As defined generally above, each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{A} is independently oxo, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is oxo. In some embodiments, R^{A} is halogen. In some embodiments, R^{A} is —CN. In some embodiments, R^{A} is —NO₂. In some embodiments, R^{A} is —OR. In some embodiments, R^{A} is —SF₅. In some embodiments, R^{A} is —SR. In some embodiments, R^{A} is —NR₂. In some embodiments, R^{A} is —S(O)₂R. In some embodiments, R^{A} is —S(O)₂NR₂. In some embodiments, R^{A} is —S(O)₂F. In some embodiments, R^{A} is —S(O)R. In some embodiments, R^{A} is —S(O)NR₂. In some embodiments, R^{A} is —S(O)(NR)R. In some embodiments, R^{A} is —C(O)R. In some embodiments, R^{A} is —C(O)OR. In some embodiments, R^{A} is —C(O)NR₂. In some embodiments, R^{A} is —C(O)N(R)OR. In some embodiments, R^{A} is —OC(O)R. In some embodiments, R^{A} is —OC(O)NR₂. In some embodiments, R^{A} is —N(R)C(O)OR. In some embodiments, R^{A} is —N(R)C(O)R. In some embodiments, R^{A} is —N(R)C(O)NR₂. In some embodiments, R^{A} is —N(R)C(NR)NR₂. In some embodiments, R^{A} is —N(R)S(O)₂NR₂. In some embodiments, R^{A} is —N(R)S(O)₂R. In some embodiments, R^{A} is —P(O)R₂. In some embodiments, R^{A} is —P(O)(R)OR. In some embodiments, R^{A} is —B(OR)₂. In some embodiments, R^{A} is deuterium.

In some embodiments, R^{A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is halogen, —CN, or —NO₂. In some embodiments, R^{A} is —OR, —SR, or —NR₂. In some embodiments, R^{A} is —S(O)₂R, —S(O)₂NR₂, —S(O)₂F, —S(O)R, —S(O)NR₂, or —S(O)(NR)R. In some embodiments, R^{A} is —C(O)R, —C(O)OR, —C(O)NR₂, or —C(O)N(R)OR. In some embodiments, R^{A} is —OC(O)R or —OC(O)NR₂. In some embodiments, R^{A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{A} is —P(O)R₂ or —P(O)(R)OR.

In some embodiments, R^{A} is —OR, —OC(O)R, or —OC(O)NR₂. In some embodiments, R^{A} is —SR, —S(O)₂R, —S(O)₂NR₂, —S(O)₂F, —S(O)R, —S(O)NR₂, or —S(O)(NR)R. In some embodiments, R^{A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{A} is —S(O)₂R, —S(O)₂NR₂, or —S(O)₂F. In some embodiments, R^{A} is —S(O)R, —S(O)NR₂, or —S(O)(NR)R. In some embodiments, R^{A} is —SR, —S(O)₂R, or —S(O)R. In some embodiments, R^{A} is —S(O)₂NR₂, —S(O)NR₂, or —S(O)(NR)R. In some embodiments, R^{A} is —S(O)₂NR₂ or —S(O)NR₂. In some embodiments, R^{A} is —SR, —S(O)₂R, —S(O)₂NR₂, or —S(O)R.

In some embodiments, R^{A} is —N(R)C(O)OR, —N(R)C(O)R, or —N(R)C(O)NR₂. In some embodiments, R^{A} is —N(R)S(O)₂NR₂ or —N(R)S(O)₂R. In some embodiments, R^{A} is —N(R)C(O)OR or —N(R)C(O)R. In some embodiments, R^{A} is —N(R)C(O)NR₂ or —N(R)S(O)₂NR₂. In some embodiments, R^{A} is —N(R)C(O)OR, —N(R)C(O)R, or —N(R)S(O)₂R.

In some embodiments, R^{A} is —NR₂, —N(R)C(O)OR, —N(R)C(O)R, or —N(R)C(O)NR₂. In some embodiments, R^{A} is —NR₂, —N(R)C(O)OR, or —N(R)C(O)R. In some embodiments, R^{A} is —NR₂, —N(R)C(O)OR, —N(R)C(O)R, or —N(R)S(O)₂R.

In some embodiments, R^{A} is selected from the groups depicted in the compounds in Table 1.

As defined generally above, each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain. In some embodiments, R^{B} is phenyl. In some embodiments, R^{B} is naphthyl. In some embodiments, R^{B} is cubanyl. In some embodiments, R^{B} is adamantyl. In some embodiments, R^{B} is a 5-6 membered monocyclic heteroaryl ring having 1-4 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring. In some embodiments, R^{B} is a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur. In some embodiments, R^{B} is a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur. In some embodiments, R^{B} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur.

In some embodiments, R^{B} is phenyl or naphthyl. In some embodiments, R^{B} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl or a 5-6 membered monocyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, R^{B} is naphthyl or an 8-10 membered bicyclic heteroaryl ring having 1-4 heteroatoms independently selected from nitrogen, oxygen, and sulfur. In some embodiments, R^{B} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur.

In some embodiments, R^{B} is phenyl or a 3-7 membered saturated or partially unsaturated monocyclic carbocyclic ring. In some embodiments, R^{B} is naphthyl or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{B} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur.

In some embodiments, R^{B} is a C₁₋₆ aliphatic chain, a 3-7 membered saturated or partially unsaturated monocyclic carbocyclic ring, or a 5-12 membered saturated or partially unsaturated bicyclic carbocyclic ring. In some embodiments, R^{B} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur. In some embodiments, R^{B} is a C₁₋₆ aliphatic chain, phenyl, or a 3-7 membered saturated or partially unsaturated monocyclic carbocyclic ring.

In some embodiments, R^{B} is selected from the groups depicted in the compounds in Table 1.

As defined generally above, each instance of R^{1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{1C} is oxo. In some embodiments, R^{1C} is deuterium. In some embodiments, each instance of R^{1C} is independently halogen. In some embodiments, R^{1C} is —CN. In some embodiments, R^{1C} is —NO₂. In some embodiments, R^{1C} is —OR. In some embodiments, R^{1C} is —SR. In some embodiments, R^{1C} is —NR₂. In some embodiments, R^{1C} is —S(O)₂R. In some embodiments, R^{1C} is —S(O)₂NR₂. In some embodiments, R^{1C} is —S(O)₂F. In some embodiments, R^{1C} is —S(O)R. In some embodiments, R^{1C} is —S(O)NR₂. In some embodiments, R^{1C} is —S(O)(NR)R. In some embodiments, R^{1C} is —C(O)R. In some embodiments, R^{1C} is —C(O)OR. In some embodiments, R^{1C} is —C(O)NR₂. In some embodiments, R^{1C} is —C(O)N(R)OR. In some embodiments, R^{1C} is —OC(O)R. In some embodiments, R^{1C} is —OC(O)NR₂. In some embodiments, R^{1C} is —N(R)C(O)OR. In some embodiments, R^{1C} is —N(R)C(O)R. In some embodiments, R^{1C} is —N(R)C(O)NR₂. In some embodiments, R^{1C} is —N(R)C(NR)NR₂. In some embodiments, R^{1C} is —N(R)S(O)₂NR₂. In some embodiments, R^{1C} is —N(R)S(O)₂R. In some embodiments, R^{1C} is —P(O)R₂. In some embodiments, R^{1C} is —P(O)(R)OR. In some embodiments, R^{1C} is —B(OR)₂.

In some embodiments, each instance of R^{1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{1C} is independently halogen, —CN, or —NO₂. In some embodiments, each instance of R^{1C} is independently —OR, —SR, or —NR₂. In some embodiments, each instance of R^{1C} is independently —S(O)₂R, —S(O)₂NR₂, —S(O)₂F, —S(O)R, —S(O)NR₂, or —S(O)(NR)R. In some embodiments, each instance of R^{1C} is independently —C(O)R, —C(O)OR, —C(O)NR₂, or —C(O)N(R)OR. In some embodiments, each instance of R^{1C} is independently —OC(O)R or —OC(O)NR₂. In some embodiments, each instance of R^{1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{1C} is independently —P(O)R₂ or —P(O)(R)OR.

In some embodiments, each instance of R^{1C} is independently —OR, —OC(O)R, or —OC(O)NR₂. In some embodiments, each instance of R^{1C} is independently —SR, —S(O)₂R, —S(O)₂NR₂, —S(O)₂F, —S(O)R, —S(O)NR₂, or —S(O)(NR)R. In some embodiments, each instance of R^{1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{1C} is independently —S(O)₂R, —S(O)₂NR₂, or —S(O)₂F. In some embodiments, each instance of R^{1C} is independently —S(O)R, —S(O)NR₂, or —S(O)(NR)R. In some embodiments, each instance of R^{1C} is independently —SR, —S(O)₂R, or —S(O)R. In some embodiments, each instance of R^{1C} is independently —S(O)₂NR₂, —S(O)NR₂, or —S(O)(NR)R. In some embodiments, each instance of R^{1C} is independently —S(O)₂NR₂ or —S(O)NR₂. In some embodiments, each instance of R^{1C} is independently —SR, —S(O)₂R, —S(O)₂NR₂, or —S(O)R.

In some embodiments, each instance of R^{1C} is independently —N(R)C(O)OR, —N(R)C(O)R, or —N(R)C(O)NR₂. In some embodiments, each instance of R^{1C} is independently —N(R)S(O)₂NR₂ or —N(R)S(O)₂R. In some embodiments, each instance of R^{1C} is independently —N(R)C(O)OR or —N(R)C(O)R. In some embodiments, each instance of R^{1C} is independently —N(R)C(O)NR₂ or —N(R)S(O)₂NR₂. In some embodiments, each instance of R^{1C} is independently —N(R)C(O)OR, —N(R)C(O)R, or —N(R)S(O)₂R.

In some embodiments, each instance of R^{1C} is independently —NR₂, —N(R)C(O)OR, —N(R)C(O)R, or —N(R)C(O)NR₂. In some embodiments, each instance of R^{1C} is independently —NR₂, —N(R)C(O)OR, or —N(R)C(O)R. In some embodiments, each instance of R^{1C} is independently —NR₂, —N(R)C(O)OR, —N(R)C(O)R, or —N(R)S(O)₂R.

In some embodiments, each instance of R^{1C} is independently an optionally substituted C₁₋₆ aliphatic. In some embodiments, each instance of R^{1C} is independently an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic. In some embodiments, R^{1C} is phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur. In some embodiments, each instance of R^{1C} is independently a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{1C} is independently phenyl or a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{1C} is independently halogen, —CN, —O-(optionally substituted C₁₋₆ aliphatic), or an optionally substituted C₁₋₆ aliphatic. In some embodiments, each instance of R^{1C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{1C} is independently halogen or C₁₋₃ aliphatic optionally substituted with 1-3 halogen. In some embodiments, each instance of R^{1C} is independently fluorine, chlorine, —CH₃, —CHF₂, or —CF₃.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{1C} is independently selected from the groups depicted in the compounds in Table 1.

As defined generally above, each instance of R^{2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{2C} is oxo. In some embodiments, R^{2C} is deuterium. In some embodiments, each instance of R^{2C} is independently halogen. In some embodiments, R^{2C} is —CN. In some embodiments, R^{2C} is —NO₂. In some embodiments, R^{2C} is —OR. In some embodiments, R^{2C} is —SR. In some embodiments, R^{2C} is —NR₂. In some embodiments, R^{2C} is —S(O)₂R. In some embodiments, R^{2C} is —S(O)₂NR₂. In some embodiments, R^{2C} is —S(O)₂F. In some embodiments, R^{2C} is —S(O)R. In some embodiments, R^{2C} is —S(O)NR₂. In some embodiments, R^{2C} is —S(O)(NR)R. In some embodiments, R^{2C} is —C(O)R. In some embodiments, R^{2C} is —C(O)OR. In some embodiments, R^{2C} is —C(O)NR₂. In some embodiments, R^{2C} is —C(O)N(R)OR. In some embodiments, R^{2C} is —OC(O)R. In some embodiments, R^{2C} is —OC(O)NR₂. In some embodiments, R^{2C} is —N(R)C(O)OR. In some embodiments, R^{2C} is —N(R)C(O)R. In some embodiments, R^{2C} is —N(R)C(O)NR₂. In some embodiments, R^{2C} is —N(R)C(NR)NR₂. In some embodiments, R^{2C} is —N(R)S(O)₂NR₂. In some embodiments, R^{2C} is —N(R)S(O)₂R. In some embodiments, R^{2C} is —P(O)R₂. In some embodiments, R^{2C} is —P(O)(R)OR. In some embodiments, R^{2C} is —B(OR)₂.

In some embodiments, each instance of R^{2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{2C} is independently halogen, —CN, or —NO₂. In some embodiments, each instance of R^{2C} is independently —OR, —SR, or —NR₂. In some embodiments, each instance of R^{2C} is independently —S(O)₂R, —S(O)₂NR₂, —S(O)₂F, —S(O)R, —S(O)NR₂, or —S(O)(NR)R. In some embodiments, each instance of R^{2C} is independently —C(O)R, —C(O)OR, —C(O)NR₂, or —C(O)N(R)OR. In some embodiments, each instance of R^{2C} is independently —OC(O)R or —OC(O)NR₂. In some embodiments, each instance of R^{2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{2C} is independently —P(O)R₂ or —P(O)(R)OR.

In some embodiments, each instance of R^{2C} is independently —OR, —OC(O)R, or —OC(O)NR₂. In some embodiments, each instance of R^{2C} is independently —SR, —S(O)₂R, —S(O)₂NR₂, —S(O)₂F, —S(O)R, —S(O)NR₂, or —S(O)(NR)R. In some embodiments, each instance of R^{2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{2C} is independently —S(O)₂R, —S(O)₂NR₂, or —S(O)₂F. In some embodiments, each instance of R^{2C} is independently —S(O)R, —S(O)NR₂, or —S(O)(NR)R. In some embodiments, each instance of R^{2C} is independently —SR, —S(O)₂R, or —S(O)R. In some embodiments, each instance of R^{2C} is independently —S(O)₂NR₂, —S(O)NR₂, or —S(O)(NR)R. In some embodiments, each instance of R^{2C} is independently —S(O)₂NR₂ or —S(O)NR₂. In some embodiments, each instance of R^{2C} is independently —SR, —S(O)₂R, —S(O)₂NR₂, or —S(O)R.

In some embodiments, each instance of R^{2C} is independently —N(R)C(O)OR, —N(R)C(O)R, or —N(R)C(O)NR₂. In some embodiments, each instance of R^{2C} is independently —N(R)S(O)₂NR₂ or —N(R)S(O)₂R. In some embodiments, each instance of R^{2C} is independently —N(R)C(O)OR or —N(R)C(O)R. In some embodiments, each instance of R^{2C} is independently —N(R)C(O)NR₂ or —N(R)S(O)₂NR₂. In some embodiments, each instance of R^{2C} is independently —N(R)C(O)OR, —N(R)C(O)R, or —N(R)S(O)₂R.

In some embodiments, each instance of R^{2C} is independently —NR₂, —N(R)C(O)OR, —N(R)C(O)R, or —N(R)C(O)NR₂. In some embodiments, each instance of R^{2C} is independently —NR₂, —N(R)C(O)OR, or —N(R)C(O)R. In some embodiments, each instance of R^{2C} is independently —NR₂, —N(R)C(O)OR, —N(R)C(O)R, or —N(R)S(O)₂R.

In some embodiments, each instance of R^{2C} is independently an optionally substituted C₁₋₆ aliphatic. In some embodiments, each instance of R^{2C} is independently an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic. In some embodiments, R^{2C} is phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur. In some embodiments, each instance of R^{2C} is independently a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{2C} is independently phenyl or a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently halogen, —CN, —O-(optionally substituted C₁₋₆ aliphatic), or an optionally substituted C₁₋₆ aliphatic. In some embodiments, each instance of R^{2C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{2C} is independently halogen or C₁₋₃ aliphatic optionally substituted with 1-3 halogen. In some embodiments, each instance of R^{2C} is independently fluorine, chlorine, —CH₃, —CHF₂, or —CF₃.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{2C} is independently selected from the groups depicted in the compounds in Table 1.

As defined generally above, each instance of R^{XC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{XC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{XC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{XC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{XC} is oxo. In some embodiments, R^{XC} is deuterium. In some embodiments, each instance of R^{XC} is independently halogen. In some embodiments, R^{XC} is —CN. In some embodiments, R^{XC} is —NO₂. In some embodiments, R^{XC} is —OR. In some embodiments, R^{XC} is —SR. In some embodiments, R^{XC} is —NR₂. In some embodiments, R^{XC} is —S(O)₂R. In some embodiments, R^{XC} is —S(O)₂NR₂. In some embodiments, R^{XC} is —S(O)₂F. In some embodiments, R^{XC} is —S(O)R. In some embodiments, R^{XC} is —S(O)NR₂. In some embodiments, R^{XC} is —S(O)(NR)R. In some embodiments, R^{XC} is —C(O)R. In some embodiments, R^{XC} is —C(O)OR. In some embodiments, R^{XC} is —C(O)NR₂. In some embodiments, R^{XC} is —C(O)N(R)OR. In some embodiments, R^{XC} is —OC(O)R. In some embodiments, R^{XC} is —OC(O)NR₂. In some embodiments, R^{XC} is —N(R)C(O)OR. In some embodiments, R^{XC} is —N(R)C(O)R. In some embodiments, R^{XC} is —N(R)C(O)NR₂. In some embodiments, R^{XC} is —N(R)C(NR)NR₂. In some embodiments, R^{XC} is —N(R)S(O)₂NR₂. In some embodiments, R^{XC} is —N(R)S(O)₂R. In some embodiments, R^{XC} is —P(O)R₂. In some embodiments, R^{XC} is —P(O)(R)OR. In some embodiments, R^{XC} is —B(OR)₂.

In some embodiments, each instance of R^{XC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{XC} is independently halogen, —CN, or —NO₂. In some embodiments, each instance of R^{XC} is independently —OR, —SR, or —NR₂. In some embodiments, each instance of R^{XC} is independently —S(O)₂R, —S(O)₂NR₂, —S(O)₂F, —S(O)R, —S(O)NR₂, or —S(O)(NR)R. In some embodiments, each instance of R^{XC} is independently —C(O)R, —C(O)OR, —C(O)NR₂, or —C(O)N(R)OR. In some embodiments, each instance of R^{XC} is independently —OC(O)R or —OC(O)NR₂. In some embodiments, each instance of R^{XC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{XC} is independently —P(O)R₂ or —P(O)(R)OR.

In some embodiments, each instance of R^{XC} is independently —OR, —OC(O)R, or —OC(O)NR₂. In some embodiments, each instance of R^{XC} is independently —SR, —S(O)₂R, —S(O)₂NR₂, —S(O)₂F, —S(O)R, —S(O)NR₂, or —S(O)(NR)R. In some embodiments, each instance of R^{XC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{XC} is independently —S(O)₂R, —S(O)₂NR₂, or —S(O)₂F. In some embodiments, each instance of R^{XC} is independently —S(O)R, —S(O)NR₂, or —S(O)(NR)R. In some embodiments, each instance of R^{XC} is independently —SR, —S(O)₂R, or —S(O)R. In some embodiments, each instance of R^{XC} is independently —S(O)₂NR₂, —S(O)NR₂, or —S(O)(NR)R. In some embodiments, each instance of R^{XC} is independently —S(O)₂NR₂ or —S(O)NR₂. In some embodiments, each instance of R^{XC} is independently —SR, —S(O)₂R, —S(O)₂NR₂, or —S(O)R.

In some embodiments, each instance of R^{XC} is independently —N(R)C(O)OR, —N(R)C(O)R, or —N(R)C(O)NR₂. In some embodiments, each instance of R^{XC} is independently —N(R)S(O)₂NR₂ or —N(R)S(O)₂R. In some embodiments, each instance of R^{XC} is independently —N(R)C(O)OR or —N(R)C(O)R. In some embodiments, each instance of R^{XC} is independently —N(R)C(O)NR₂ or —N(R)S(O)₂NR₂. In some embodiments, each instance of R^{XC} is independently —N(R)C(O)OR, —N(R)C(O)R, or —N(R)S(O)₂R.

In some embodiments, each instance of R^{XC} is independently —NR₂, —N(R)C(O)OR, —N(R)C(O)R, or —N(R)C(O)NR₂. In some embodiments, each instance of R^{XC} is independently —NR₂, —N(R)C(O)OR, or —N(R)C(O)R. In some embodiments, each instance of R^{XC} is independently —NR₂, —N(R)C(O)OR, —N(R)C(O)R, or —N(R)S(O)₂R.

In some embodiments, each instance of R^{XC} is independently an optionally substituted C₁₋₆ aliphatic. In some embodiments, each instance of R^{XC} is independently an optionally substituted phenyl. In some embodiments, each instance of R^{XC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{XC} is independently an optionally substituted 5-6 membered monocyclic heteroaryl ring having 1-4 heteroatoms independently selected from nitrogen, oxygen, and sulfur.

In some embodiments, each instance of R^{XC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{XC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{XC} is independently an optionally substituted C₁₋₆ aliphatic or an optionally substituted phenyl. In some embodiments, each instance of R^{XC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{XC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{XC} is independently a C₁₋₆ aliphatic. In some embodiments, R^{XC} is phenyl. In some embodiments, each instance of R^{XC} is independently a 3-7 membered saturated or partially unsaturated monocyclic heterocyclic ring having 1-2 heteroatoms independently selected from nitrogen, oxygen, and sulfur. In some embodiments, each instance of R^{XC} is independently a 5-6 membered monocyclic heteroaryl ring having 1-4 heteroatoms independently selected from nitrogen, oxygen, and sulfur.

In some embodiments, each instance of R^{XC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{XC} is independently phenyl or a 5-6 membered monocyclic heteroaryl ring having 1-4 heteroatoms independently selected from nitrogen, oxygen, and sulfur.

In some embodiments, each instance of R^{XC} is independently a C₁₋₆ aliphatic or phenyl. In some embodiments, each instance of R^{XC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{XC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{XC} is independently selected from the groups depicted in the compounds in Table 1.

As defined generally above, each instance of R^{YC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{YC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{YC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{YC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{YC} is oxo. In some embodiments, R^{YC} is deuterium. In some embodiments, each instance of R^{YC} is independently halogen. In some embodiments, R^{YC} is —CN. In some embodiments, R^{YC} is —NO₂. In some embodiments, R^{YC} is —OR. In some embodiments, R^{YC} is —SR. In some embodiments, R^{YC} is —NR₂. In some embodiments, R^{YC} is —S(O)₂R. In some embodiments, R^{YC} is —S(O)₂NR₂. In some embodiments, R^{YC} is —S(O)₂F. In some embodiments, R^{YC} is —S(O)R. In some embodiments, R^{YC} is —S(O)NR₂. In some embodiments, R^{YC} is —S(O)(NR)R. In some embodiments, R^{YC} is —C(O)R. In some embodiments, R^{YC} is —C(O)OR. In some embodiments, R^{YC} is —C(O)NR₂. In some embodiments, R^{YC} is —C(O)N(R)OR. In some embodiments, R^{YC} is —OC(O)R. In some embodiments, R^{YC} is —OC(O)NR₂. In some embodiments, R^{YC} is —N(R)C(O)OR. In some embodiments, R^{YC} is —N(R)C(O)R. In some embodiments, R^{YC} is —N(R)C(O)NR₂. In some embodiments, R^{YC} is —N(R)C(NR)NR₂. In some embodiments, R^{YC} is —N(R)S(O)₂NR₂. In some embodiments, R^{YC} is —N(R)S(O)₂R. In some embodiments, R^{YC} is —P(O)R₂. In some embodiments, R^{YC} is —P(O)(R)OR. In some embodiments, R^{YC} is —B(OR)₂.

In some embodiments, each instance of R^{YC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{YC} is independently halogen, —CN, or —NO₂. In some embodiments, each instance of R^{YC} is independently —OR, —SR, or —NR₂. In some embodiments, each instance of R^{YC} is independently —S(O)₂R, —S(O)₂NR₂, —S(O)₂F, —S(O)R, —S(O)NR₂, or —S(O)(NR)R. In some embodiments, each instance of R^{YC} is independently —C(O)R, —C(O)OR, —C(O)NR₂, or —C(O)N(R)OR. In some embodiments, each instance of R^{YC} is independently —OC(O)R or —OC(O)NR₂. In some embodiments, each instance of R^{YC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{YC} is independently —P(O)R₂ or —P(O)(R)OR.

In some embodiments, each instance of R^{YC} is independently —OR, —OC(O)R, or —OC(O)NR₂. In some embodiments, each instance of R^{YC} is independently —SR, —S(O)₂R, —S(O)₂NR₂, —S(O)₂F, —S(O)R, —S(O)NR₂, or —S(O)(NR)R. In some embodiments, each instance of R^{YC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{YC} is independently —S(O)₂R, —S(O)₂NR₂, or —S(O)₂F. In some embodiments, each instance of R^{YC} is independently —S(O)R, —S(O)NR₂, or —S(O)(NR)R. In some embodiments, each instance of R^{YC} is independently —SR, —S(O)₂R, or —S(O)R. In some embodiments, each instance of R^{YC} is independently —S(O)₂NR₂, —S(O)NR₂, or —S(O)(NR)R. In some embodiments, each instance of R^{YC} is independently —S(O)₂NR₂ or —S(O)NR₂. In some embodiments, each instance of R^{YC} is independently —SR, —S(O)₂R, —S(O)₂NR₂, or —S(O)R.

In some embodiments, each instance of R^{YC} is independently —N(R)C(O)OR, —N(R)C(O)R, or —N(R)C(O)NR₂. In some embodiments, each instance of R^{YC} is independently —N(R)S(O)₂NR₂ or —N(R)S(O)₂R. In some embodiments, each instance of R^{YC} is independently —N(R)C(O)OR or —N(R)C(O)R. In some embodiments, each instance of R^{YC} is independently —N(R)C(O)NR₂ or —N(R)S(O)₂NR₂. In some embodiments, each instance of R^{YC} is independently —N(R)C(O)OR, —N(R)C(O)R, or —N(R)S(O)₂R.

In some embodiments, each instance of R^{YC} is independently —NR₂, —N(R)C(O)OR, —N(R)C(O)R, or —N(R)C(O)NR₂. In some embodiments, each instance of R^{YC} is independently —NR₂, —N(R)C(O)OR, or —N(R)C(O)R. In some embodiments, each instance of R^{YC} is independently —NR₂, —N(R)C(O)OR, —N(R)C(O)R, or —N(R)S(O)₂R.

In some embodiments, each instance of R^{YC} is independently an optionally substituted C₁₋₆ aliphatic. In some embodiments, each instance of R^{YC} is independently an optionally substituted phenyl. In some embodiments, each instance of R^{YC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{YC} is independently an optionally substituted 5-6 membered monocyclic heteroaryl ring having 1-4 heteroatoms independently selected from nitrogen, oxygen, and sulfur.

In some embodiments, each instance of R^{YC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{YC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{YC} is independently an optionally substituted C₁₋₆ aliphatic or an optionally substituted phenyl. In some embodiments, each instance of R^{YC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{YC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{YC} is independently a C₁₋₆ aliphatic. In some embodiments, R^{YC} is phenyl. In some embodiments, each instance of R^{YC} is independently a 3-7 membered saturated or partially unsaturated monocyclic heterocyclic ring having 1-2 heteroatoms independently selected from nitrogen, oxygen, and sulfur. In some embodiments, each instance of R^{YC} is independently a 5-6 membered monocyclic heteroaryl ring having 1-4 heteroatoms independently selected from nitrogen, oxygen, and sulfur.

In some embodiments, each instance of R^{YC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{YC} is independently phenyl or a 5-6 membered monocyclic heteroaryl ring having 1-4 heteroatoms independently selected from nitrogen, oxygen, and sulfur.

In some embodiments, each instance of R^{YC} is independently a C₁₋₆ aliphatic or phenyl. In some embodiments, each instance of R^{YC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{YC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{YC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{YC} is independently halogen, —CN, —OH, —O-(optionally substituted C₁₋₃ aliphatic), or an optionally substituted C₁₋₃ aliphatic. In some embodiments, each instance of R^{YC} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{YC} is independently fluorine, chlorine, —OH, —OCH₃, —OCF₃, —CH₃, —CHF₂, or —CF₃. In some embodiments, each instance of R^{YC} is independently fluorine or —OH.

In some embodiments, each instance of R^{YC} is independently oxo, deuterium, halogen, —CN, —OH, —O-(optionally substituted C₁₋₃ aliphatic), or an optionally substituted C₁₋₃ aliphatic. In some embodiments, each instance of R^{YC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{YC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{YC} is independently oxo, deuterium, fluorine, chlorine, —CN, —OH, —OCH₃, —OCF₃, —CH₃, —CHF₂, or —CF₃. In some embodiments, each instance of R^{YC} is independently oxo, deuterium, —CN, fluorine, or —OH. In some embodiments, each instance of R^{YC} is independently oxo, deuterium, —CN, —CH₃, or —CHF₂. In some embodiments, each instance of R^{YC} is independently deuterium, —CN, —CH₃, or —CHF₂.

In some embodiments, each instance of R^{YC} is independently oxo, halogen, —CN, —OH, —O-(optionally substituted C₁₋₃ aliphatic), or an optionally substituted C₁₋₃ aliphatic. In some embodiments, each instance of R^{YC} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{YC} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{YC} is independently oxo, fluorine, chlorine, —CN, —OH, —OCH₃, —OCF₃, —CH₃, —CHF₂, or —CF₃. In some embodiments, each instance of R^{YC} is independently oxo, —CN, fluorine, or —OH. In some embodiments, each instance of R^{YC} is independently oxo, —CN, —CH₃, or —CHF₂. In some embodiments, each instance of R^{YC} is independently —CN, —CH₃, or —CHF₂.

In some embodiments, each instance of R^{YC} is independently selected from the groups depicted in the compounds in Table 1.

As defined generally above, each instance of R^{LC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{LC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{LC} is oxo. In some embodiments, R^{LC} is deuterium. In some embodiments, each instance of R^{LC} is independently halogen. In some embodiments, R^{LC} is —CN. In some embodiments, R^{LC} is —NO₂. In some embodiments, R^{LC} is —OR. In some embodiments, R^{LC} is —SR. In some embodiments, R^{LC} is —NR₂. In some embodiments, R^{LC} is —S(O)₂R. In some embodiments, R^{LC} is —S(O)₂NR₂. In some embodiments, R^{LC} is —S(O)₂F. In some embodiments, R^{LC} is —S(O)R. In some embodiments, R^{LC} is —S(O)NR₂. In some embodiments, R^{LC} is —S(O)(NR)R. In some embodiments, R^{LC} is —C(O)R. In some embodiments, R^{LC} is —C(O)OR. In some embodiments, R^{LC} is —C(O)NR₂. In some embodiments, R^{LC} is —C(O)N(R)OR. In some embodiments, R^{LC} is —OC(O)R. In some embodiments, R^{LC} is —OC(O)NR₂. In some embodiments, R^{LC} is —N(R)C(O)OR. In some embodiments, R^{LC} is —N(R)C(O)R. In some embodiments, R^{LC} is —N(R)C(O)NR₂. In some embodiments, R^{LC} is —N(R)C(NR)NR₂. In some embodiments, R^{LC} is —N(R)S(O)₂NR₂. In some embodiments, R^{LC} is —N(R)S(O)₂R. In some embodiments, R^{LC} is —P(O)R₂. In some embodiments, R^{LC} is —P(O)(R)OR. In some embodiments, R^{LC} is —B(OR)₂.

In some embodiments, each instance of R^{LC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{LC} is independently halogen, —CN, or —NO₂. In some embodiments, each instance of R^{LC} is independently —OR, —SR, or —NR₂. In some embodiments, each instance of R^{LC} is independently —S(O)₂R, —S(O)₂NR₂, —S(O)₂F, —S(O)R, —S(O)NR₂, or —S(O)(NR)R. In some embodiments, each instance of R^{LC} is independently —C(O)R, —C(O)OR, —C(O)NR₂, or —C(O)N(R)OR. In some embodiments, each instance of R^{LC} is independently —OC(O)R or —OC(O)NR₂. In some embodiments, each instance of R^{LC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{LC} is independently —P(O)R₂ or —P(O)(R)OR.

In some embodiments, each instance of R^{LC} is independently —OR, —OC(O)R, or —OC(O)NR₂. In some embodiments, each instance of R^{LC} is independently —SR, —S(O)₂R, —S(O)₂NR₂, —S(O)₂F, —S(O)R, —S(O)NR₂, or —S(O)(NR)R. In some embodiments, each instance of R^{LC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{LC} is independently —S(O)₂R, —S(O)₂NR₂, or —S(O)₂F. In some embodiments, each instance of R^{LC} is independently —S(O)R, —S(O)NR₂, or —S(O)(NR)R. In some embodiments, each instance of R^{LC} is independently —SR, —S(O)₂R, or —S(O)R. In some embodiments, each instance of R^{LC} is independently —S(O)₂NR₂, —S(O)NR₂, or —S(O)(NR)R. In some embodiments, each instance of R^{LC} is independently —S(O)₂NR₂ or —S(O)NR₂. In some embodiments, each instance of R^{LC} is independently —SR, —S(O)₂R, —S(O)₂NR₂, or —S(O)R.

In some embodiments, each instance of R^{LC} is independently —N(R)C(O)OR, —N(R)C(O)R, or —N(R)C(O)NR₂. In some embodiments, each instance of R^{LC} is independently —N(R)S(O)₂NR₂ or —N(R)S(O)₂R. In some embodiments, each instance of R^{LC} is independently —N(R)C(O)OR or —N(R)C(O)R. In some embodiments, each instance of R^{LC} is independently —N(R)C(O)NR₂ or —N(R)S(O)₂NR₂. In some embodiments, each instance of R^{LC} is independently —N(R)C(O)OR, —N(R)C(O)R, or —N(R)S(O)₂R.

In some embodiments, each instance of R^{LC} is independently —NR₂, —N(R)C(O)OR, —N(R)C(O)R, or —N(R)C(O)NR₂. In some embodiments, each instance of R^{LC} is independently —NR₂, —N(R)C(O)OR, or —N(R)C(O)R. In some embodiments, each instance of R^{LC} is independently —NR₂, —N(R)C(O)OR, —N(R)C(O)R, or —N(R)S(O)₂R.

In some embodiments, each instance of R^{LC} is independently an optionally substituted C₁₋₆ aliphatic. In some embodiments, each instance of R^{LC} is independently an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic. In some embodiments, R^{LC} is phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur. In some embodiments, each instance of R^{LC} is independently a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{LC} is independently phenyl or a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently selected from the groups depicted in the compounds in Table 1.

As defined generally above, each instance of R^{CyAC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{CyAC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{CyAC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{CyAC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{CyAC} is oxo. In some embodiments, R^{CyAC} is deuterium. In some embodiments, each instance of R^{CyAC} is independently halogen. In some embodiments, R^{CyAC} is —CN. In some embodiments, R^{CyAC} is —NO₂. In some embodiments, R^{CyAC} is —OR. In some embodiments, R^{CyAC} is —SR. In some embodiments, R^{CyAC} is —NR₂. In some embodiments, R^{CyAC} is —S(O)₂R. In some embodiments, R^{CyAC} is —S(O)₂NR₂. In some embodiments, R^{CyAC} is —S(O)₂F. In some embodiments, R^{CyAC} is —S(O)R. In some embodiments, R^{CyAC} is —S(O)NR₂. In some embodiments, R^{CyAC} is —S(O)(NR)R. In some embodiments, R^{CyAC} is —C(O)R. In some embodiments, R^{CyAC} is —C(O)OR. In some embodiments, R^{CyAC} is —C(O)NR₂. In some embodiments, R^{CyAC} is —C(O)N(R)OR. In some embodiments, R^{CyAC} is —OC(O)R. In some embodiments, R^{CyAC} is —OC(O)NR₂. In some embodiments, R^{CyAC} is —N(R)C(O)OR. In some embodiments, R^{CyAC} is —N(R)C(O)R. In some embodiments, R^{CyAC} is —N(R)C(O)NR₂. In some embodiments, R^{CyAC} is —N(R)C(NR)NR₂. In some embodiments, R^{CyAC} is —N(R)S(O)₂NR₂. In some embodiments, R^{CyAC} is —N(R)S(O)₂R. In some embodiments, R^{CyAC} is —P(O)R₂. In some embodiments, R^{CyAC} is —P(O)(R)OR. In some embodiments, R^{CyAC} is —B(OR)₂.

In some embodiments, each instance of R^{CyAC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{CyAC} is independently halogen, —CN, or —NO₂. In some embodiments, each instance of R^{CyAC} is independently —OR, —SR, or —NR₂. In some embodiments, each instance of R^{CyAC} is

independently —S(O)₂R, —S(O)₂NR₂, —S(O)₂F, —S(O)R,
—S(O)NR₂, or —S(O)(NR)R. In some embodiments, each instance of R^{CyAC} is independently —C(O)R, —C(O)OR, —C(O)NR₂, or —C(O)N(R)OR. In some embodiments, each instance of R^{CyAC} is independently —OC(O)R or —OC(O)NR₂. In some embodiments, each instance of R^{CyAC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{CyAC} is independently —P(O)R₂ or —P(O)(R)OR.

In some embodiments, each instance of R^{CyAC} is independently —OR, —OC(O)R, or —OC(O)NR₂. In some embodiments, each instance of R^{CyAC} is independently —SR, —S(O)₂R, —S(O)₂NR₂, —S(O)₂F, —S(O)R, —S(O)NR₂, or —S(O)(NR)R. In some embodiments, each instance of R^{CyAC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{CyAC} is independently —S(O)₂R, —S(O)₂NR₂, or —S(O)₂F. In some embodiments, each instance of R^{CyAC} is

independently —S(O)R, —S(O)NR₂, or —S(O)(NR)R. In some embodiments, each instance of R^{CyAC} is independently —SR, —S(O)₂R, or —S(O)R. In some embodiments, each instance of R^{CyAC} is independently —S(O)₂NR₂,
—S(O)NR₂, or —S(O)(NR)R. In some embodiments, each instance of R^{CyAC} is independently —S(O)₂NR₂ or —S(O)NR₂. In some embodiments, each instance of R^{CyAC} is independently —SR, —S(O)₂R, —S(O)₂NR₂, or —S(O)R.

In some embodiments, each instance of R^{CyAC} is independently —N(R)C(O)OR, —N(R)C(O)R, or —N(R)C(O)NR₂. In some embodiments, each instance of R^{CyAC} is independently —N(R)S(O)₂NR₂ or —N(R)S(O)₂R. In some embodiments, each instance of R^{CyAC} is independently —N(R)C(O)OR or —N(R)C(O)R. In some embodiments, each instance of R^{CyAC} is independently —N(R)C(O)NR₂ or —N(R)S(O)₂NR₂. In some embodiments, each instance of R^{CyAC} is independently —N(R)C(O)OR, —N(R)C(O)R, or —N(R)S(O)₂R.

In some embodiments, each instance of R^{CyAC} is independently —NR₂, —N(R)C(O)OR, —N(R)C(O)R, or —N(R)C(O)NR₂. In some embodiments, each instance of R^{CyAC} is independently —NR₂, —N(R)C(O)OR, or —N(R)C(O)R. In some embodiments, each instance of R^{CyAC} is independently —NR₂, —N(R)C(O)OR, —N(R)C(O)R, or —N(R)S(O)₂R.

In some embodiments, each instance of R^{CyAC} is independently an optionally substituted C₁₋₆ aliphatic. In some embodiments, each instance of R^{CyAC} is independently an optionally substituted phenyl. In some embodiments, each instance of R^{CyAC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{CyAC} is independently an optionally substituted 5-6 membered monocyclic heteroaryl ring having 1-4 heteroatoms independently selected from nitrogen, oxygen, and sulfur.

In some embodiments, each instance of R^{CyAC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{CyAC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{CyAC} is independently an optionally substituted C₁₋₆ aliphatic or an optionally substituted phenyl. In some embodiments, each instance of R^{CyAC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{CyAC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{CyAC} is independently a C₁₋₆ aliphatic. In some embodiments, R^{CyAC} is phenyl. In some embodiments, each instance of R^{CyAC} is independently a 3-7 membered saturated or partially unsaturated monocyclic heterocyclic ring having 1-2 heteroatoms independently selected from nitrogen, oxygen, and sulfur. In some embodiments, each instance of R^{CyAC} is independently a 5-6 membered monocyclic heteroaryl ring having 1-4 heteroatoms independently selected from nitrogen, oxygen, and sulfur.

In some embodiments, each instance of R^{CyAC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{CyAC} is independently phenyl or a 5-6 membered monocyclic heteroaryl ring having 1-4 heteroatoms independently selected from nitrogen, oxygen, and sulfur.

In some embodiments, each instance of R^{CyAC} is independently a C₁₋₆ aliphatic or phenyl. In some embodiments, each instance of R^{CyAC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{CyAC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{CyAC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{CyAC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen atoms. In some embodiments, each instance of R^{CyAC} is independently oxo, deuterium, fluorine, chlorine, —CN, —OH, —OCH₃, —OCHF₂, —OCF₃, —CH₃, —CHF₂, or —CF₃.

In some embodiments, each instance of R^{CyAC} is independently halogen, —CN, —O-(optionally substituted C₁₋₆ aliphatic), or an optionally substituted C₁₋₆ aliphatic. In some embodiments, each instance of R^{CyAC} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{CyAC} is independently halogen or C₁₋₃ aliphatic optionally substituted with 1-3 halogen. In some embodiments, each instance of R^{CyAC} is independently fluorine, chlorine, —CH₃, —CHF₂, or —CF₃.

In some embodiments, each instance of R^{CyAC} is independently selected from the groups depicted in the compounds in Table 1.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R is hydrogen, C₁₋₆ aliphatic, phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is an optionally substituted C₁₋₆ aliphatic. In some embodiments, R is an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, R is an optionally substituted phenyl or an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, R is an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is a C₁₋₆ aliphatic. In some embodiments, R is phenyl. In some embodiments, R is a 3-7 membered saturated or partially unsaturated monocyclic heterocyclic ring having 1-2 heteroatoms independently selected from nitrogen, oxygen, and sulfur. In some embodiments, R is a 5-6 membered monocyclic heteroaryl ring having 1-4 heteroatoms independently selected from nitrogen, oxygen, and sulfur.

In some embodiments, R is a C₁₋₆ aliphatic or a 3-7 membered saturated or partially unsaturated monocyclic heterocyclic ring having 1-2 heteroatoms independently selected from nitrogen, oxygen, and sulfur. In some embodiments, R is phenyl or a 5-6 membered monocyclic heteroaryl ring having 1-4 heteroatoms independently selected from nitrogen, oxygen, and sulfur.

In some embodiments, R is a C₁₋₆ aliphatic or phenyl. In some embodiments, R is a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, two R groups on the same nitrogen are taken together with their intervening atoms to form a 4-7 membered saturated, partially unsaturated, or heteroaryl ring having 1-3 heteroatoms, in addition to the nitrogen, independently selected from nitrogen, oxygen, and sulfur. In some embodiments, two R groups on the same nitrogen are taken together with their intervening atoms to form a 4-7 membered saturated, partially unsaturated, or heteroaryl ring having no additional heteroatoms other than said nitrogen.

In some embodiments, two R groups on the same nitrogen are taken together with their intervening atoms to form a 4-7 membered saturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 0-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 1-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having no additional heteroatoms other than said nitrogen. In some embodiments, two R groups on the same nitrogen are taken together with their intervening atoms to form a 4-7 membered partially unsaturated ring having no additional heteroatoms other than said nitrogen. In some embodiments, two R groups on the same nitrogen are taken together with their intervening atoms to form a 4-7 membered heteroaryl ring having no additional heteroatoms other than said nitrogen.

In some embodiments, R is selected from the groups depicted in the compounds in Table 1.

As defined generally above, n is 0, 1, 2, 3, 4, or 5. In some embodiments, n is 0. In some embodiments, n is 1. In some embodiments, n is 2. In some embodiments, n is 3. In some embodiments, n is 4. In some embodiments, n is 5. In some embodiments, n is 0 or 1. In some embodiments, n is 0, 1, or 2. In some embodiments, n is 0, 1, 2, or 3. In some embodiments, n is 0, 1, 2, 3, or 4. In some embodiments, n is 1 or 2. In some embodiments, n is 1, 2, or 3. In some embodiments, n is 1, 2, 3, or 4. In some embodiments, n is 1, 2, 3, 4, or 5. In some embodiments, n is 2 or 3. In some embodiments, n is 2, 3, or 4. In some embodiments, n is 2, 3, 4, or 5. In some embodiments, n is 3 or 4. In some embodiments, n is 3, 4, or 5. In some embodiments, n is 4 or 5. In some embodiments, n is selected from the values represented in the compounds in Table 1.

As defined generally above, r¹ is 0, 1, 2, 3, 4, or 5. In some embodiments, r¹ is 0. In some embodiments, r¹ is 1. In some embodiments, r¹ is 2. In some embodiments, r¹ is 3. In some embodiments, r¹ is 4. In some embodiments, r¹ is 5. In some embodiments, r¹ is 0 or 1. In some embodiments, r¹ is 0, 1, or 2. In some embodiments, r¹ is 0, 1, 2, or 3. In some embodiments, r¹ is 0, 1, 2, 3, or 4. In some embodiments, r¹ is 1 or 2. In some embodiments, r¹ is 1, 2, or 3. In some embodiments, r¹ is 1, 2, 3, or 4. In some embodiments, r¹ is 1, 2, 3, 4, or 5. In some embodiments, r¹ is 2 or 3. In some embodiments, r¹ is 2, 3, or 4. In some embodiments, r¹ is 2, 3, 4, or 5. In some embodiments, r¹ is 3 or 4. In some embodiments, r¹ is 3, 4, or 5. In some embodiments, r¹ is 4 or 5. In some embodiments, r¹ is selected from the values represented in the compounds in Table 1.

As defined generally above, r² is 0, 1, 2, 3, 4, or 5. In some embodiments, r² is 0. In some embodiments, r² is 1. In some embodiments, r² is 2. In some embodiments, r² is 3. In some embodiments, r² is 4. In some embodiments, r² is 5. In some embodiments, r² is 0 or 1. In some embodiments, r² is 0, 1, or 2. In some embodiments, r² is 0, 1, 2, or 3. In some embodiments, r² is 0, 1, 2, 3, or 4. In some embodiments, r² is 1 or 2. In some embodiments, r² is 1, 2, or 3. In some embodiments, r² is 1, 2, 3, or 4. In some embodiments, r² is 1, 2, 3, 4, or 5. In some embodiments, r² is 2 or 3. In some embodiments, r² is 2, 3, or 4. In some embodiments, r² is 2, 3, 4, or 5. In some embodiments, r² is 3 or 4. In some embodiments, r² is 3, 4, or 5. In some embodiments, r² is 4 or 5. In some embodiments, r² is selected from the values represented in the compounds in Table 1.

As defined generally above, r³ is 0, 1, 2, 3, 4, or 5. In some embodiments, r³ is 0. In some embodiments, r³ is 1. In some embodiments, r³ is 2. In some embodiments, r³ is 3. In some embodiments, r³ is 4. In some embodiments, r³ is 5. In some embodiments, r³ is 0 or 1. In some embodiments, r³ is 0, 1, or 2. In some embodiments, r³ is 0, 1, 2, or 3. In some embodiments, r³ is 0, 1, 2, 3, or 4. In some embodiments, r³ is 1 or 2. In some embodiments, r³ is 1, 2, or 3. In some embodiments, r³ is 1, 2, 3, or 4. In some embodiments, r³ is 1, 2, 3, 4, or 5. In some embodiments, r³ is 2 or 3. In some embodiments, r³ is 2, 3, or 4. In some embodiments, r³ is 2, 3, 4, or 5. In some embodiments, r³ is 3 or 4. In some embodiments, r³ is 3, 4, or 5. In some embodiments, r³ is 4 or 5. In some embodiments, r³ is selected from the values represented in the compounds in Table 1.

As defined generally above, r⁴ is 0, 1, 2, 3, 4, or 5. In some embodiments, r⁴ is 0. In some embodiments, r⁴ is 1. In some embodiments, r⁴ is 2. In some embodiments, r⁴ is 3. In some embodiments, r⁴ is 4. In some embodiments, r⁴ is 5. In some embodiments, r⁴ is 0 or 1. In some embodiments, r⁴ is 0, 1, or 2. In some embodiments, r⁴ is 0, 1, 2, or 3. In some embodiments, r⁴ is 0, 1, 2, 3, or 4. In some embodiments, r⁴ is 1 or 2. In some embodiments, r⁴ is 1, 2, or 3. In some embodiments, r⁴ is 1, 2, 3, or 4. In some embodiments, r⁴ is 1, 2, 3, 4, or 5. In some embodiments, r⁴ is 2 or 3. In some embodiments, r⁴ is 2, 3, or 4. In some embodiments, r⁴ is 2, 3, 4, or 5. In some embodiments, r⁴ is 3 or 4. In some embodiments, r⁴ is 3, 4, or 5. In some embodiments, r⁴ is 4 or 5. In some embodiments, r⁴ is selected from the values represented in the compounds in Table 1.

As defined generally above, r⁵ is 0, 1, 2, 3, 4, or 5. In some embodiments, r⁵ is 0. In some embodiments, r⁵ is 1. In some embodiments, r⁵ is 2. In some embodiments, r⁵ is 3. In some embodiments, r⁵ is 4. In some embodiments, r⁵ is 5. In some embodiments, r⁵ is 0 or 1. In some embodiments, r⁵ is 0, 1, or 2. In some embodiments, r⁵ is 0, 1, 2, or 3. In some embodiments, r⁵ is 0, 1, 2, 3, or 4. In some embodiments, r⁵ is 1 or 2. In some embodiments, r⁵ is 1, 2, or 3. In some embodiments, r⁵ is 1, 2, 3, or 4. In some embodiments, r⁵ is 1, 2, 3, 4, or 5. In some embodiments, r⁵ is 2 or 3. In some embodiments, r⁵ is 2, 3, or 4. In some embodiments, r⁵ is 2, 3, 4, or 5. In some embodiments, r⁵ is 3 or 4. In some embodiments, r⁵ is 3, 4, or 5. In some embodiments, r⁵ is 4 or 5. In some embodiments, r⁵ is selected from the values represented in the compounds in Table 1.

As defined generally above, r⁶ is 0, 1, 2, 3, 4, or 5. In some embodiments, r⁶ is 0. In some embodiments, r⁶ is 1. In some embodiments, r⁶ is 2. In some embodiments, r⁶ is 3. In some embodiments, r⁶ is 4. In some embodiments, r⁶ is 5. In some embodiments, r⁶ is 0 or 1. In some embodiments, r⁶ is 0, 1, or 2. In some embodiments, r⁶ is 0, 1, 2, or 3. In some embodiments, r⁶ is 0, 1, 2, 3, or 4. In some embodiments, r⁶ is 1 or 2. In some embodiments, r⁶ is 1, 2, or 3. In some embodiments, r⁶ is 1, 2, 3, or 4. In some embodiments, r⁶ is 1, 2, 3, 4, or 5. In some embodiments, r⁶ is 2 or 3. In some embodiments, r⁶ is 2, 3, or 4. In some embodiments, r⁶ is 2, 3, 4, or 5. In some embodiments, r⁶ is 3 or 4. In some embodiments, r⁶ is 3, 4, or 5. In some embodiments, r⁶ is 4 or 5. In some embodiments, r⁶ is selected from the values represented in the compounds in Table 1.

In some embodiments, the present disclosure provides a compound of formula I, wherein Cy^{A} is selected from embodiments herein, forming a compound of formulas II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX and XXXI:

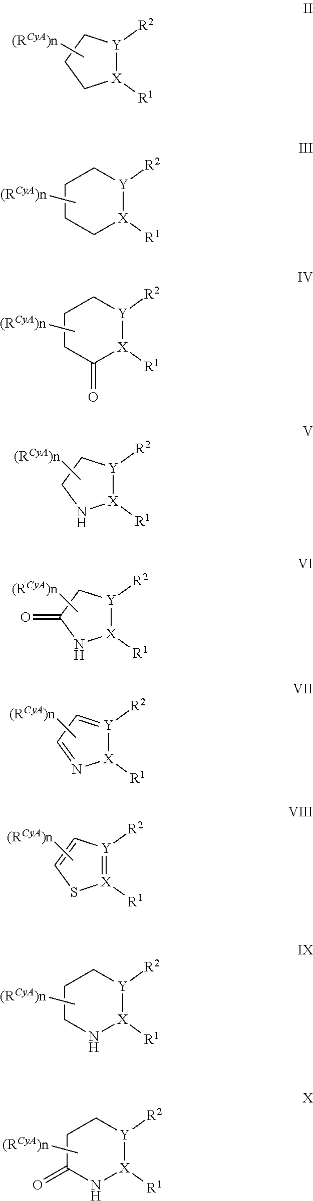

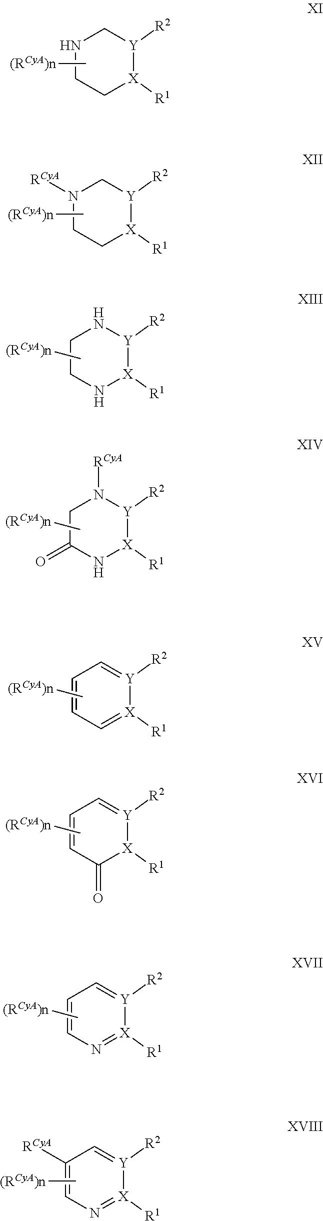

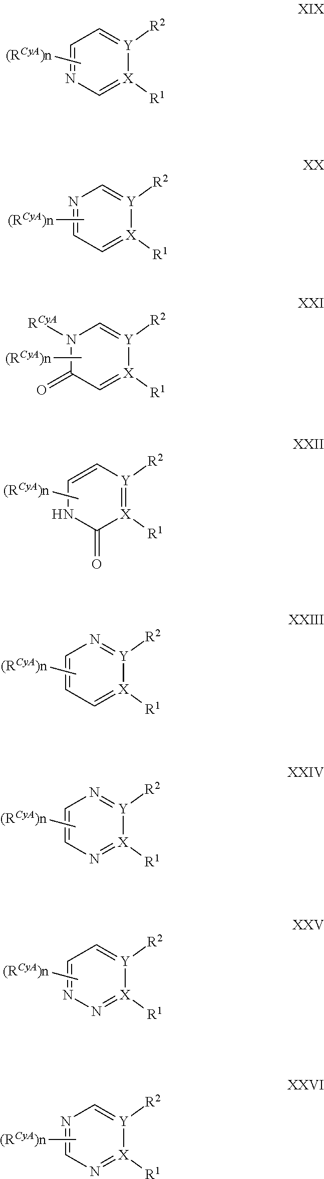

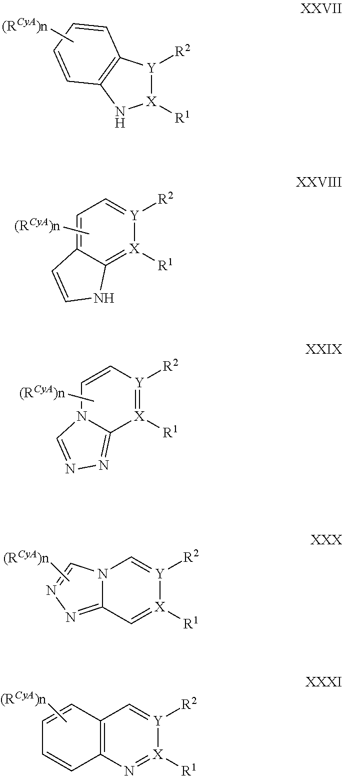

or a pharmaceutically acceptable salt thereof, wherein each of R¹, R², R^{CyA}, X, Y, and n is as defined in embodiments and classes and subclasses herein.

In some embodiments, the present disclosure provides a compound of formula I wherein X is C and Y is C, forming a compound of formulas XXXII, XXXIII, XXXIV, XXXV, XXXVI, XXXVII, XXXVIII, XXXIX, XL, XLI, XLII, XLIII, XLIV, XLV, and XLVI:

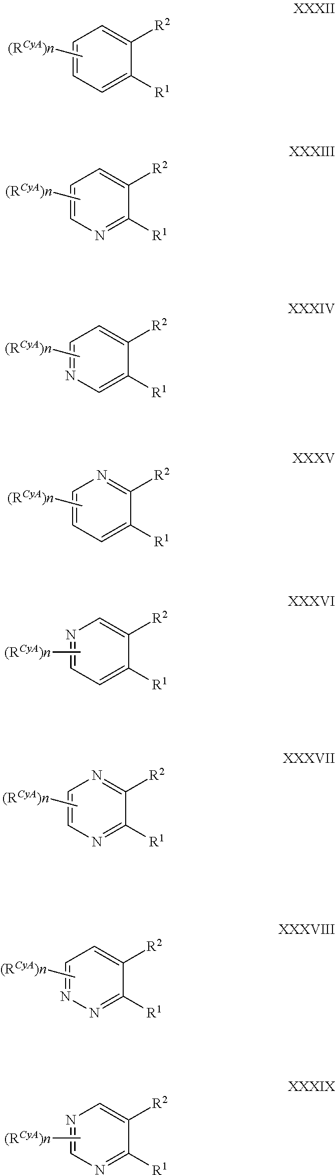

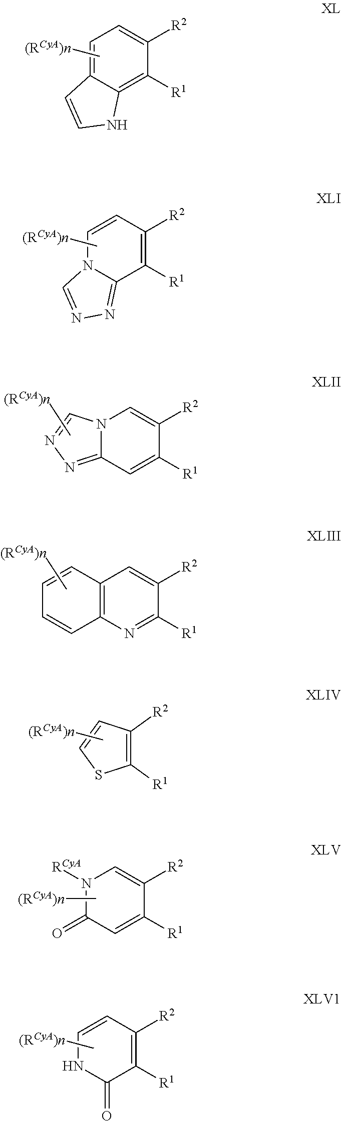

or a pharmaceutically acceptable salt thereof, wherein each of R¹, R², R^{CyA}, and n is as defined in embodiments and classes and subclasses herein.

In some embodiments, the present disclosure provides a compound of formula I wherein X is CH and Y in CH, forming a compound of formulas XLVII, XLVIII, XLIX, L, LI, and LII:

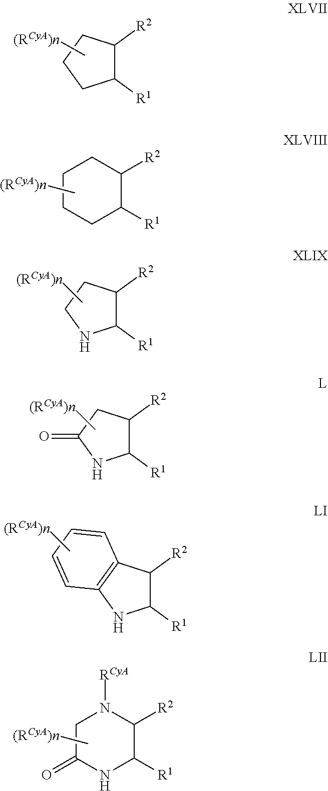

or a pharmaceutically acceptable salt thereof, wherein each of R¹, R², R^{CyA}, and n is as defined in embodiments and classes and subclasses herein.

In some embodiments, the present disclosure provides a compound of formula I wherein X is N and Y is C or CH, forming a compound of formulas LIII, LIV, and LV:

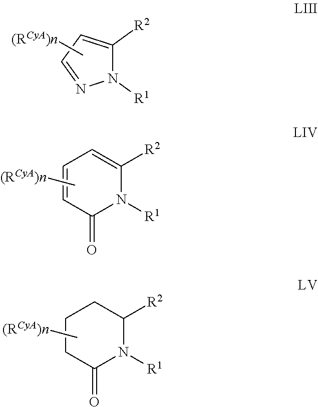

or a pharmaceutically acceptable salt thereof, wherein each of R¹, R², R^{CyA}, and n is as defined in embodiments and classes and subclasses herein.

In some embodiments, the present disclosure provides a compound of formula XXXIII wherein n is 1, forming a compound of formulas LVI, LVII, and LVIII:

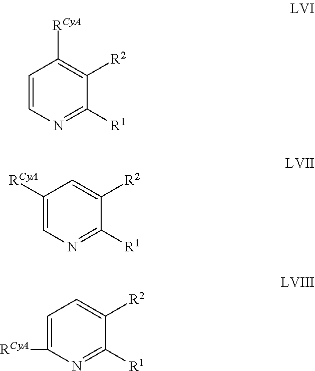

or a pharmaceutically acceptable salt thereof, wherein each of R¹, R², and R^{CyA} is independently as defined in embodiments and classes and subclasses herein.

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein L¹ is —N(H)—, and R² is —N(R)C(O)—R^{2A}, —N(R)—R^{2A}, or —R^{2A}.

In some embodiments, the present disclosure provides a compound of I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein L¹ is —N(H)—, and R² is —N(R)C(O)—R^{2A}. In some embodiments, the present disclosure provides a compound of I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein L¹ is —N(H)—, and R² is —N(R)—R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein L¹ is —N(H)—, and R² is —R^{2A}.

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein L¹ is —N(H)— (i.e. R¹ is —N(H)—R^{1A}).

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein R² is —N(R)C(O)—R^{2A}, —N(R)—R^{2A}, or —R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein R² is —N(R)C(O)—R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein R² is —N(R)—R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein R² is —R^{2A}.

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein R² is —N(H)C(O)—R^{2A}, —N(H)—R^{2A}, or —R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein R² is —N(H)C(O)—R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, wherein R² is —N(H)—R^{2A}.

Examples of compounds of the present disclosure include those listed in the Tables and exemplification herein, or a pharmaceutically acceptable salt, stereoisomer, or mixture of stereoisomers thereof. In some embodiments, the present disclosure provides a compound selected from those depicted in Table 1, below, or a pharmaceutically acceptable salt, stereoisomer, or mixture of stereoisomers thereof. In some embodiments, the present disclosure provides a compound set forth in Table 1, below, or a pharmaceutically acceptable salt thereof. In some embodiments, the present disclosure provides a compound set forth in Table 1, below.

TABLE 1
Representative Compounds of the Disclosure with Bioactivity Data.

| | | | | ADP- | MCF |
|---|---|---|---|---|---|
| Ex. | | | | Glo | 10A |
| No. | Structure | ¹H NMR | MS | IC₅₀ | I₅₀ |
| I-1 | 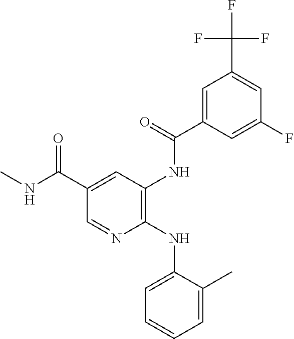 [SMILES: CNC(=O)c1cnc(Nc2ccccc2C)c(NC(=O)c2cc(F)cc(C(F)(F)F)c2)c1] | (DMSO-d6, 400 MHz) 10.29 (1H, s), 8.43 (1H, d, J = 2.2 Hz), 8.29 (2H, s), 8.23 (1H, s), 8.18 (1H, d, J = 9.1 Hz), 8.03-7.96 (2H, m), 7.32 (1H, dd, J = 7.9, 1.4 Hz), 7.27-7.22 (1H, m), 7.19 (1H, td, J = 7.6, 1.7 Hz), 7.09 (1H, td, J = 7.4, 1.4 Hz), 2.77 (3H, d, J = 4.5 Hz), 2.16 (3H, s) | 447.05 | C | C |
| I-2 | 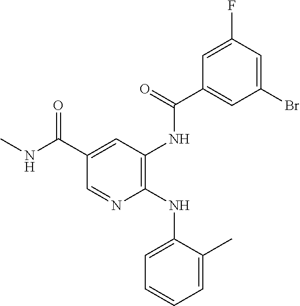 [SMILES: CNC(=O)c1cnc(Nc2ccccc2C)c(NC(=O)c2cc(F)cc(Br)c2)c1] | | 457.25 | C | |
| I-3 | 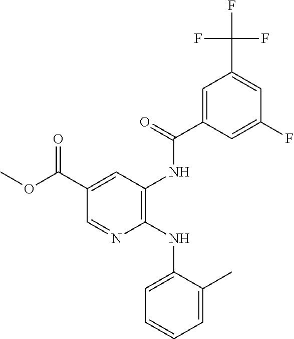 [SMILES: COC(=O)c1cnc(Nc2ccccc2C)c(NC(=O)c2cc(F)cc(C(F)(F)F)c2)c1] | | 448.29 | D | |
| I-4 | 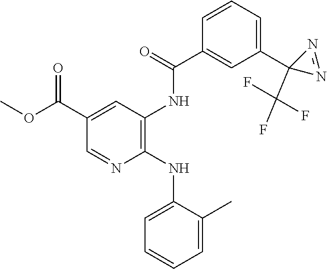 [SMILES: COC(=O)c1cnc(Nc2ccccc2C)c(NC(=O)c2cccc(C3(C(F)(F)F)N=N3)c2)c1] | | 469.35 | D | |
| I-5 | 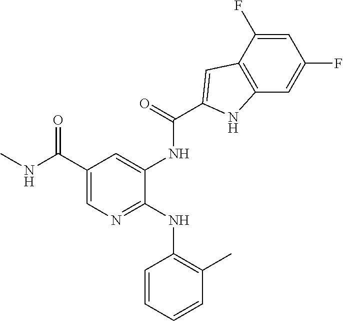 [SMILES: CNC(=O)c1cnc(Nc2ccccc2C)c(NC(=O)c2cc3c(F)cc(F)cc3[nH]2)c1] | | | E | |
| I-6 | 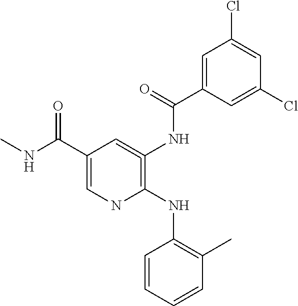 | | | C | C |
| I-7 | 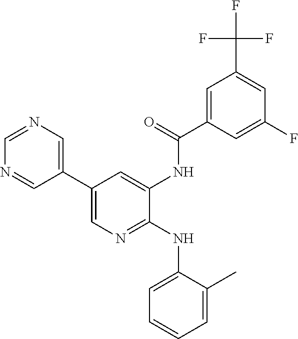 [SMILES: Cc1ccccc1Nc1ncc(-c2cncnc2)cc1NC(=O)c1cc(F)cc(C(F)(F)F)c1] | | | B | C |
| I-8 | 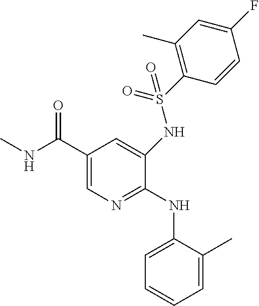 [SMILES: CNC(=O)c1cnc(Nc2ccccc2C)c(NS(=O)(=O)c2ccc(F)cc2C)c1] | | | B | B |
| I-9 | 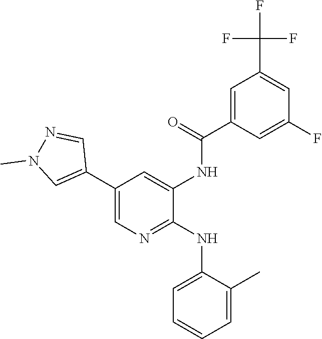 [SMILES: Cc1ccccc1Nc1ncc(-c2cnn(C)c2)cc1NC(=O)c1cc(F)cc(C(F)(F)F)c1] | (400 MHz, cd3od) 8.18 (d, J = 2.2 Hz, 1H), 8.11 (s, 1H), 7.96 (d, J = 8.7 Hz, 1H), 7.91 (s, 1H), 7.87 (d, J = 2.2 Hz, 1H), 7.77 (s, 1H), 7.71 (d, J = 8.3 Hz, 1H), 7.29 (d, J = 7.9 Hz, 1H), 7.19 (d, J = 7.4 Hz, 1H), 7.12 (t, J = 7.1 Hz, 1H), 7.02-6.97 (m, 1H), 3.91 (s, 3H), 2.23 (s, 3H). | 470.4 | E | |
| I-10 | 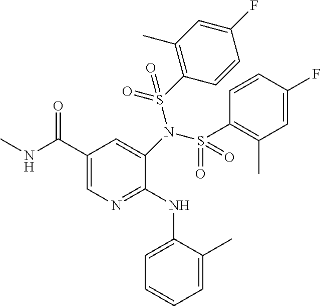 [SMILES: CNC(=O)c1cnc(Nc2ccccc2C)c(N(S(=O)(=O)c2ccc(F)cc2C)S(=O)(=O)c2ccc(F)cc2C)c1] | | | E | |
| I-11 | 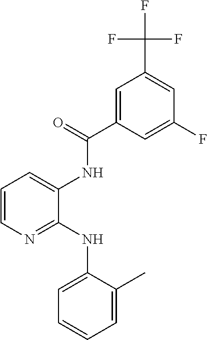 [SMILES: Cc1ccccc1Nc1ncccc1NC(=O)c1cc(F)cc(C(F)(F)F)c1] | | | D | D |
| I-12 | 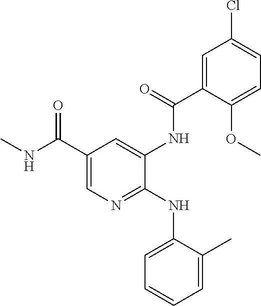 | | | E | |
| I-13 | 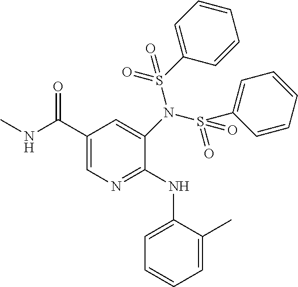 [SMILES: CNC(=O)c1cnc(Nc2ccccc2C)c(N(S(=O)(=O)c2ccccc2)S(=O)(=O)c2ccccc2)c1] | | | E | |
| I-14 | 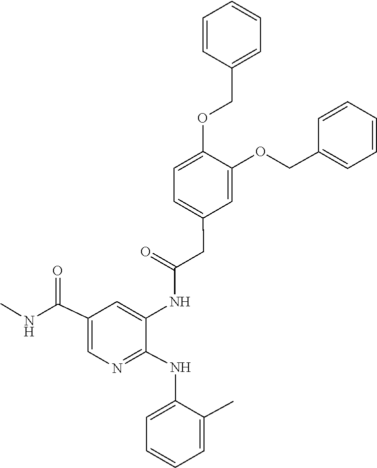 [SMILES: CNC(=O)c1cnc(Nc2ccccc2C)c(NC(=O)Cc2ccc(OCc3ccccc3)c(OCc3ccccc3)c2)c1] | | | E | |
| I-15 | 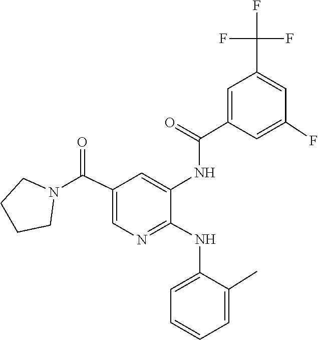 [SMILES: Cc1ccccc1Nc1ncc(C(=O)N2CCCC2)cc1NC(=O)c1cc(F)cc(C(F)(F)F)c1] | | | E | |
| I-16 | 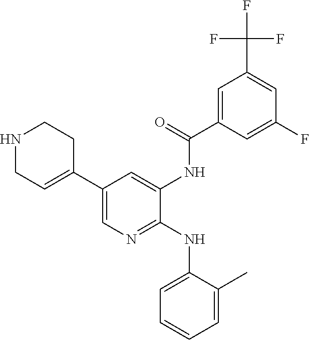 [SMILES: Cc1ccccc1Nc1ncc(C2=CCNCC2)cc1NC(=O)c1cc(F)cc(C(F)(F)F)c1] | | | E | |
| I-17 | 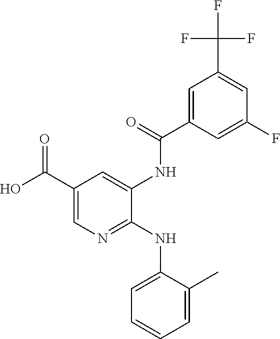 [SMILES: Cc1ccccc1Nc1ncc(C(=O)O)cc1NC(=O)c1cc(F)cc(C(F)(F)F)c1] | | | D | |
| I-18 | 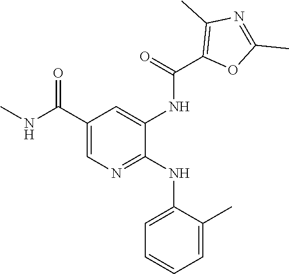 | | | D | |
| I-19 | 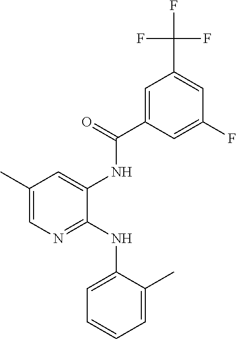 [SMILES: Cc1cnc(Nc2ccccc2C)c(NC(=O)c2cc(F)cc(C(F)(F)F)c2)c1] | | | E | |
| I-20 | 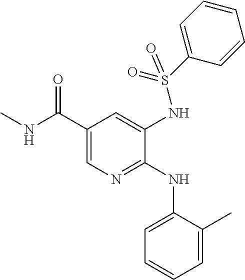 [SMILES: CNC(=O)c1cnc(Nc2ccccc2C)c(NS(=O)(=O)c2ccccc2)c1] | | | D | |
| I-21 | 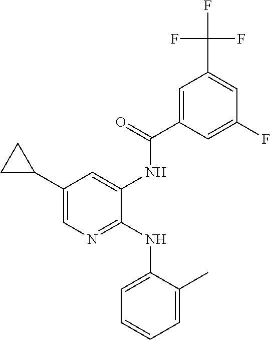 [SMILES: Cc1ccccc1Nc1ncc(C2CC2)cc1NC(=O)c1cc(F)cc(C(F)(F)F)c1] | | | D | D |
| I-22 | 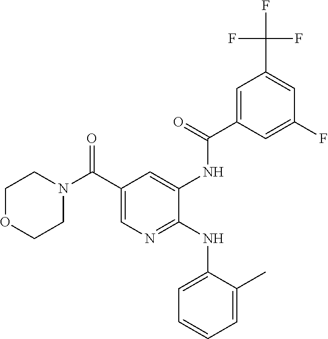 [SMILES: Cc1ccccc1Nc1ncc(C(=O)N2CCOCC2)cc1NC(=O)c1cc(F)cc(C(F)(F)F)c1] | | | E | |
| I-23 | 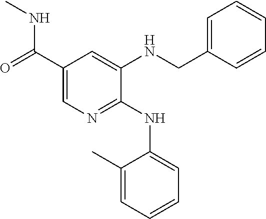 [SMILES: CNC(=O)c1cnc(Nc2ccccc2C)c(NCc2ccccc2)c1] | | | E | |
| I-24 | 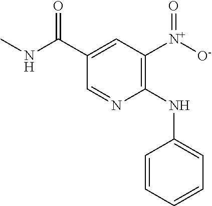 [SMILES: CNC(=O)c1cnc(Nc2ccccc2)c([N+](=O)[O-])c1] | | | E | |
| I-25 | 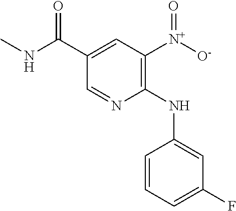 [SMILES: CNC(=O)c1cnc(Nc2cccc(F)c2)c([N+](=O)[O-])c1] | | | E | |
| I-26 | 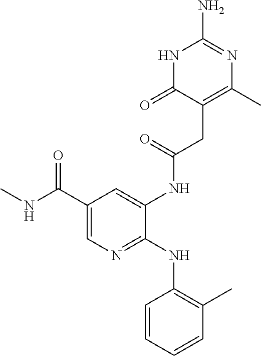 | | | E | |
| I-27 | 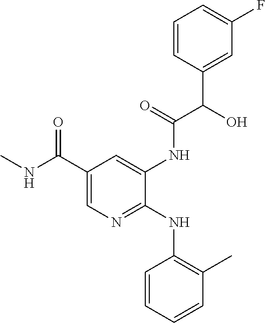 [SMILES: CNC(=O)c1cnc(Nc2ccccc2C)c(NC(=O)C(O)c2cccc(F)c2)c1] | | | C | B |
| I-28 | 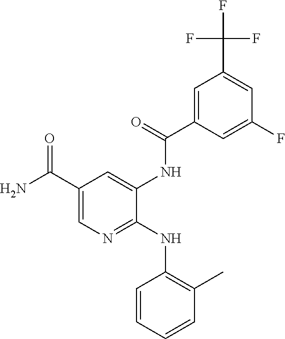 [SMILES: Cc1ccccc1Nc1ncc(C(N)=O)cc1NC(=O)c1cc(F)cc(C(F)(F)F)c1] | | | C | C |
| I-29 | 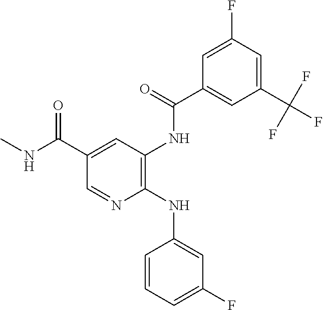 [SMILES: CNC(=O)c1cnc(Nc2cccc(F)c2)c(NC(=O)c2cc(F)cc(C(F)(F)F)c2)c1] | | | E | |
| I-30 | 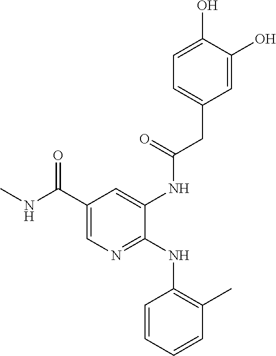 [SMILES: CNC(=O)c1cnc(Nc2ccccc2C)c(NC(=O)Cc2ccc(O)c(O)c2)c1] | | | C | C |
| I-31 | 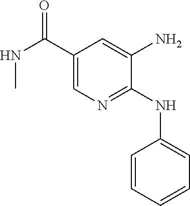 [SMILES: CNC(=O)c1cnc(Nc2ccccc2)c(N)c1] | | | E | |
| I-32 | 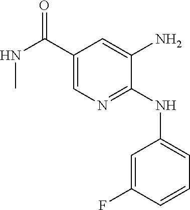 [SMILES: CNC(=O)c1cnc(Nc2cccc(F)c2)c(N)c1] | | | E | |
| I-33 | 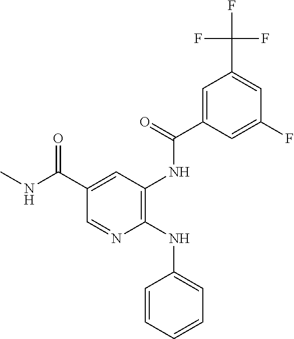 [SMILES: CNC(=O)c1cnc(Nc2ccccc2)c(NC(=O)c2cc(F)cc(C(F)(F)F)c2)c1] | | | E | |
| I-34 | 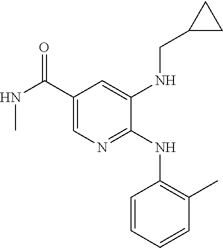 [SMILES: CNC(=O)c1cnc(Nc2ccccc2C)c(NCC2CC2)c1] | (400 MHz, cd3od) 7.84 (d, J = 2.1 Hz, 1H), 7.25 (d, J = 7.8 Hz, 2H), 7.20 (d, J = 2.1 Hz, 1H), 7.17 (d, J = 7.8 Hz, 1H), 7.10 (t, J = 7.8 Hz, 1H), 3.06 (d, J = 6.8 Hz, 2H), 2.87 (s, 3H), 2.19 (s, 3H), 1.21 (m, 1H), 0.64-0.56 (m, 2H), 0.33- 0.30 (m, 2H). | 311.32 | E | |
| I-35 | 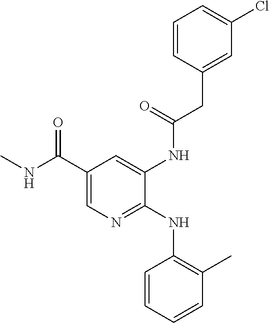 [SMILES: CNC(=O)c1cnc(Nc2ccccc2C)c(NC(=O)Cc2cccc(Cl)c2)c1] | (400 MHz, cdcl3) 8.42 (d, J = 2.0 Hz, 1H), 8.06 (d, J = 2.0 Hz, 1H), 7.39-7.27 (m, 3H), 7.25-7.20 (m, 2H), 7.15 (t, J = 7.6 Hz, 1H), 7.10-6.98 (m, 2H), 6.79 (s, 1H), 6.11 (s, 1H), 3.66 (s, 2H), 2.97 (t, J = 4.8 Hz, 3H), 2.17 (s, 3H). | 409.38 | E | |
| I-36 | 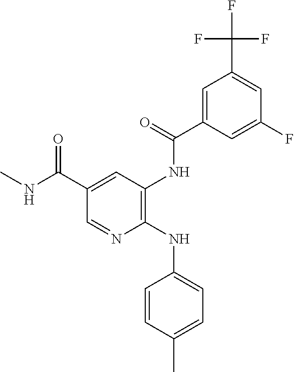 [SMILES: CNC(=O)c1cnc(Nc2ccc(C)cc2)c(NC(=O)c2cc(F)cc(C(F)(F)F)c2)c1] | 1H-NMR (400 MHz, DMSO-d6) 10.20 (s, 1H), 8.55 (d, J = 16.5 Hz, 2H), 8.38- 8.12 (m, 3H), 8.00 (d, J = 11.9 Hz, 2H), 7.52 (d, J = 8.3 Hz, 2H), 7.11 (d, J = 8.3 Hz, 2H), 5.76 (s, 0.3H, DCM), 2.77 (d, J = 4.4 Hz, 3H), 2.26 (s, 3H). | 447.4 | C | D |
| I-37 | 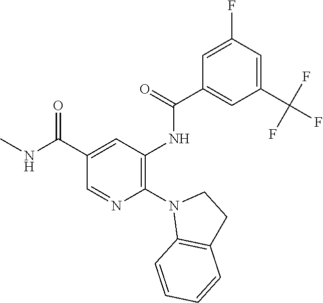 [SMILES: CNC(=O)c1cnc(N2CCc3ccccc32)c(NC(=O)c2cc(F)cc(C(F)(F)F)c2)c1] | (400 MHz, dmso) 10.31 (s, 1H), 8.68 (d, J = 2.2 Hz, 1H), 8.52 (d, J = 4.6 Hz, 1H), 8.29 (d, J = 2.2 Hz, 1H), 7.95-7.87 (m, 2H), 7.84 (d, J = 8.7 Hz, 1H), 7.17 (d, J = 7.0 Hz, 1H), 6.95 (t, J = 7.7 Hz, 1H), 6.79-6.64 (m, 2H), 5.76 (s, 0.1H, DCM), 4.08 (t, J = 8.3 Hz, 2H), 3.09 (t, J = 8.2 Hz, 2H), 2.81 (d, J = 4.5 Hz, 3H). | 459.42 | E | |
| I-38 | 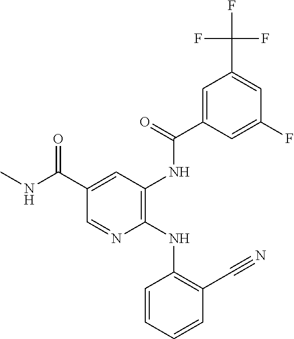 [SMILES: CNC(=O)c1cnc(Nc2ccccc2C#N)c(NC(=O)c2cc(F)cc(C(F)(F)F)c2)c1] | (400 MHz, CD3OD) 8.48 (s, 2.5H), 8.33 (d, J = 8.2 Hz, 1H), 8.15 (d, J = 2.6 Hz, 1H), 8.09 (d, J = 2.5 Hz, 1H), 7.87 (t, J = 7.2 Hz, 1H), 7.74 (d, J = 7.8 Hz, 1H), 7.67 (s, 1H), 7.65 (d, = 8 Hz, 1H), 7.61 (d, J = 8.8 Hz, 1H), 7.54 (d, J = 8.3 Hz, 1H), 2.85 (s, 3H), 2.66 (s, 0.5H). | 458.34 | E | |
| I-39 | 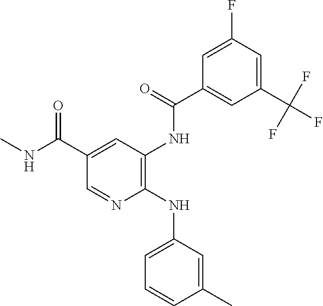 [SMILES: CNC(=O)c1cnc(Nc2cccc(C)c2)c(NC(=O)c2cc(F)cc(C(F)(F)F)c2)c1] | (400 MHz, dmso) 10.15 (s, 1H), 8.54- 8.46 (d, J = 4.3 Hz, 2H), 8.27 (m 1H), 8.19 (s, 1H), 8.12 (d, J = 9.6 Hz, 1H), 7.97 (s, 1H), 7.93 (d, J = 8.2 Hz, 1H), 7.43 (d, J = 8.0 Hz, 1H), 7.36 (s, 1H), 7.11 (t, J = 7.8 Hz, 1H), 6.75 (d, J = 7.7 Hz, 1H), 2.71 (d, J = 4.4 Hz, 3H), 2.21 (s, 3H). | 447.4 | D | |
| I-40 | 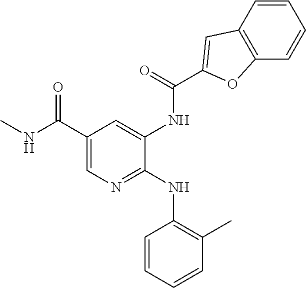 [SMILES: CNC(=O)c1cnc(Nc2ccccc2C)c(NC(=O)c2cc3ccccc3o2)c1] | (400 MHz, CDCl3) 8.49 (d, J = 2.1 Hz, 1H), 8.23 (d, J = 2.1 Hz, 1H), 8.19 (s, 1H), 7.68 (d, J = 7.8 Hz, 1H), 7.57 (s, 1H), 7.50-7.42 (m, 2H), 7.40 (d, J = 7.8 Hz, 1H), 7.31 (td, J = 7.8, 1.7 Hz, 1H), 7.21 (m, 1H), 7.16 (td, J = 7.8, 1.7 Hz, 1H), 7.06 (s, 1H), 7.02 (td, J = 7.8, 1.7 Hz, 1H), 6.03 (s, 1H), 2.99 (d, J = 4.8 Hz, 3H), 2.29 (s, 3H). | 401.39 | E | |
| I-41 | 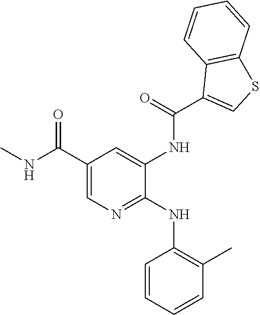 [SMILES: CNC(=O)c1cnc(Nc2ccccc2C)c(NC(=O)c2csc3ccccc23)c1] | (400 MHz, DMSO-d6) 10.04 (s, 1H), 8.69 (s, 1H), 8.48 (d, J = 7.33 Hz, 1H), 8.42 (d, J = 2.02 Hz, 1H), 8.28-8.34 (m, 1H), 8.20 (s, 1H), 8.06-8.12 (m, 2H), 7.41-7.52 (m, 3H), 7.21 (d, J = 7.07 Hz, 1H), 7.17 (t, J = 7.45 Hz, 1H), 7.02-7.08 (m, 1H), 2.76 (d, J = 4.55 Hz, 3H), 2.16 (s, 3H) | 417.2 | B | D |
| I-42 | 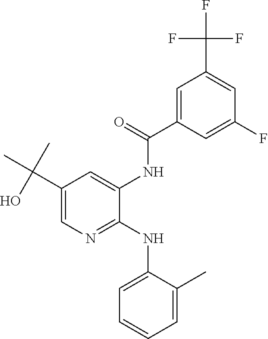 [SMILES: Cc1ccccc1Nc1ncc(C(C)(C)O)cc1NC(=O)c1cc(F)cc(C(F)(F)F)c1] | (400 MHz, cdcl3) 8.46 (d, J = 2.1 Hz, 1H), 8.33 (d, J = 2.3 Hz, 1H), 7.62 (s, 1H), 7.45 (d, J = 8.0 Hz, 1H), 7.36 (s, 1H), 7.35-7.24 (m, 1H but with CDCl3), 7.10 (t, J = 7.0 Hz, 1H), 6.99 (dd, J = 7.6, 6.2 Hz, 1H), 6.93 (d, J = 7.7 Hz, 1H), 6.21 (s, 1H), 2.35 (s, 3H), 1.66 (s, 6H). | 448.4 | D | D |
| I-43 | 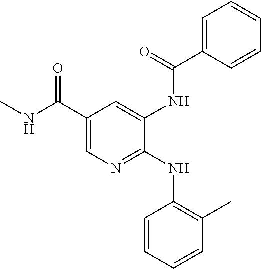 [SMILES: CNC(=O)c1cnc(Nc2ccccc2C)c(NC(=O)c2ccccc2)c1] | (400 MHz, CDCl3) 8.52 (d, J = 2.2 Hz, 1H), 8.27 (d, J = 2.2 Hz, 1H), 7.84 (s, 1H), 7.73-7.66 (m, 2H), 7.58 ((tt, J = 7.6, 1.2 Hz, 1H), 7.46 (t, J = 7.6 Hz, 2H), 7.40 (d, J = 7.6 Hz, 1H), 7.25-7.23 (m, 1H), 7.19 (t, J = 7.6 Hz, 1H), 7.06 (td, J = 7.6, 1.2 Hz, 1H), 7.00 (s, 1H), 6.07 (s, 1H), 3.01 (d, J = 4.9 Hz, 3H), 2.30 (s, 3H). | 361.38 | E | |
| I-44 | 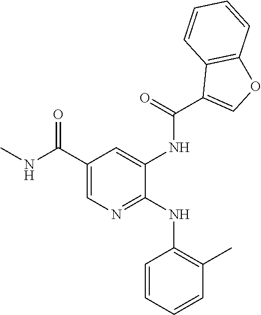 [SMILES: CNC(=O)c1cnc(Nc2ccccc2C)c(NC(=O)c2coc3ccccc23)c1] | (400 MHz, CDCl3) 8.49 (d, J = 2.1 Hz, 1H), 8.30 (d, J = 2.1 Hz, 1H), 7.97 (s, 1H), 7.83 (d, J = 7.2 Hz, 1H), 7.79 (s, 1H), 7.53 (d, J = 7.2 Hz, 1H), 7.42-7.29 (m, 3H), 7.21 (m, 1H), 7.16 (td, J = 7.2, 1.8 Hz, 1H), 7.01 (td, J = 7.2, 1.8 Hz, 1H), 6.97 (s, 1H), 6.07 (s, 1H), 2.96 (d, J = 4.8 Hz, 3H), 2.29 (s, 3H). | 401.41 | D | |
| I-45 | 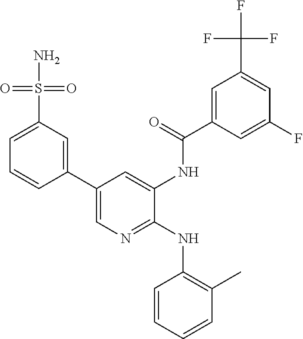 [SMILES: Cc1ccccc1Nc1ncc(-c2cccc(S(N)(=O)=O)c2)cc1NC(=O)c1cc(F)cc(C(F)(F)F)c1] | (400 MHz, dmso) 10.38 (s, 1H), 8.36 (d, J = 2.1 Hz, 1H), 8.26 (s, 1H), 8.19 (d, J = 9.0 Hz, 1H), 8.14 (s, 1H), 8.07 (t, J = 1.7 Hz, 1H), 8.05 (s, 1H), 7.99 (d, J = 8.5 Hz, 1H), 7.92-7.87 (m, 1H), 7.78-7.73 (m, 1H), 7.64 (t, J = 7.8 Hz, 1H), 7.45 (s, 1H), 7.39 (s, 2H), 7.23 (d, J = 7.4 Hz, 1H), 7.18 (t, J = 7.0 Hz, 1H), 7.04 (t, J = 7.0 Hz, 1H), 2.21 (s, 3H) | 545.4 | B | B |
| I-46 | 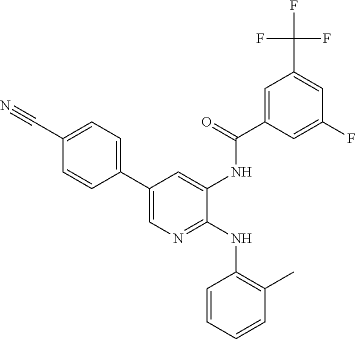 [SMILES: Cc1ccccc1Nc1ncc(-c2ccc(C#N)cc2)cc1NC(=O)c1cc(F)cc(C(F)(F)F)c1] | (400 MHz, dmso) 8.43 (d, J = 2.4 Hz, 1H), 8.31 (br s, 1H), 8.27-8.22 (m, 2H), 8.19 (d, J = 9.2 Hz, 1H), 8.11 (d, J = 2.1 Hz, 1H), 7.98 (d, J = 8.6 Hz, 1H), 7.91-7.84 (m, 4H), 7.41 (d, J = 7.7 Hz, 1H), 7.23 (d, J = 7.3 Hz, 1H), 7.18 (td, J = 7.6, 1.2 Hz, 1H), 7.06 (td, J = 7.4, 1.2 Hz, 1H), 2.20 (s, 3H) | 491.39 | B | B |
| I-47 | 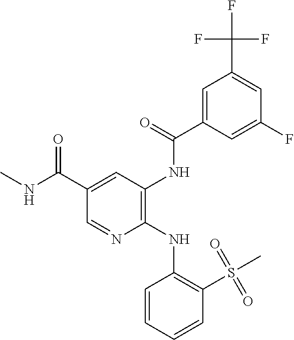 [SMILES: CNC(=O)c1cnc(Nc2ccccc2S(C)(=O)=O)c(NC(=O)c2cc(F)cc(C(F)(F)F)c2)c1] | (400 MHz, DMSO-d6) 10.70 (s, 1H), 9.30 (s, 1H), 8.75 (d, J = 8.5 Hz, 1H), 8.71 (d, J = 2.2 Hz, 1H), 8.52 (m, 1H), 8.25 (s, 1H), 8.16 (d, J = 2.2 Hz, 1H), 8.13 (d, J = 8.6 Hz, 1H), 8.04 (d, J = 8.6 Hz, 1H), 7.84 (dd, J = 8.0, 1.6 Hz, 1H), 7.78-7.68 (m, 1H), 7.29-7.14 (m, 1H), 6.54 (s, 0.24H), 3.18 (s, 3H), 2.81 (d, J = 4.5 Hz, 3H). | 511.31 | E | |
| I-48 | 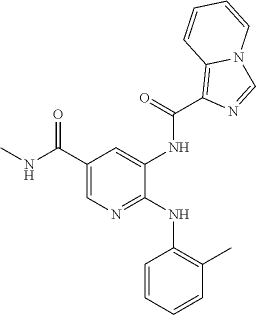 [SMILES: CNC(=O)c1cnc(Nc2ccccc2C)c(NC(=O)c2ncn3ccccc23)c1] | (400 MHz, CDCl3) 8.89 (s, 1H), 8.48 (d, J = 2.2 Hz, 1H), 8.30 (d, J = 9.2 Hz, 1H), 8.14 (d, J = 2.2 Hz, 1H), 8.07- 8.05 (m, 2H), 7.61 (d, J = 8.0 Hz, 1H), 7.54 (s, 1H), 7.22-7.10 (m, 3H), 7.01 (td, J = 6.8, 1.2 Hz, 1H), 6.83 (td, J = 6.8, 1.2 Hz, 1H), 6.03 (m, 1H), 3.00 (d, J = 4.9 Hz, 3H), 2.30 (s, 3H). | 401.37 | E | |
| I-49 | 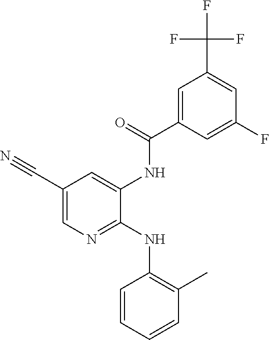 [SMILES: Cc1ccccc1Nc1ncc(C#N)cc1NC(=O)c1cc(F)cc(C(F)(F)F)c1] | 8.42 (d, J = 2.0 Hz, 1H), 8.20 (d, J = 2.0 Hz, 1H), 7.54-7.50 (m, 3H), 7.47 (dt, J = 8.2, 2.0 Hz, 1H), 7.29 (d, J = 8.2 Hz, 1H), 7.23-7.11 (m, 3H), 6.81 (s, 1H), 2.31 (s, 3H). | 415.34 | D | |
| I-50 | 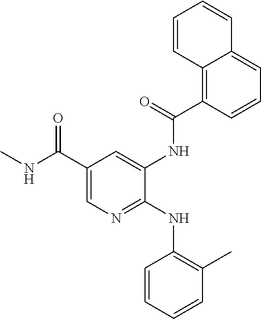 [SMILES: CNC(=O)c1cnc(Nc2ccccc2C)c(NC(=O)c2cccc3ccccc23)c1] | 9.30 (s, 1H), 8.57 (s, 1H), 8.49 (d, J = 1.9 Hz, 1H), 8.44 (d, J = 8.4 Hz, 1H), 8.35-8.32 (m, 2H), 8.03 (d, J = 8.4 Hz, 1H), 7.82 (ddd, J = 8.4, 6.8, 1.2 Hz, 1H), 7.71 (ddd, J = 8.4, 6.8, 1.2 Hz, 1H), 7.44 (d, J = 8.4 Hz, 1H), 7.25- 7.15 (m, 2H), 7.12 (s, 1H), 7.05 (td, J = 8.4, 1.2 Hz, 1H), 6.12 (m, 1H), 2.94 (d, J = 4.8 Hz, 3H), 2.30 (s, 3H). | 412.41 | E | |
| I-51 | 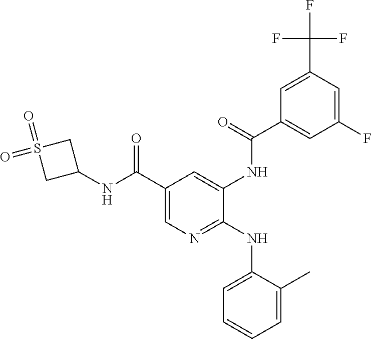 [SMILES: Cc1ccccc1Nc1ncc(C(=O)NC2CS(=O)(=O)C2)cc1NC(=O)c1cc(F)cc(C(F)(F)F)c1] | (400 MHz, cdcl3 + 2 drops MeOD) 8.44 (d, J = 2.2 Hz, 1H), 7.97 (s, 1H), 7.90 (d, J = 2.2 Hz, 1H), 7.82 (d, J = 8.7 Hz, 1H), 7.52 (d, J = 7.9 Hz, 1H), 7.40 (dd, J = 7.9, 1.1 Hz, 1H), 7.22- 7.12 (m, 2H), 7.04 (td, J = 7.4, 1.2 Hz, 1H), 4.68 (dq, J = 8.2, 4.8 Hz, 1H), 4.56-4.46 (m, 2H), 4.24-4.15 (m, 2H), 2.93 (s, 1H), 2.84 (d, J = 0.6 Hz, 1H), 2.23 (s, 3H). | 537.41 | A | A |
| I-52 | 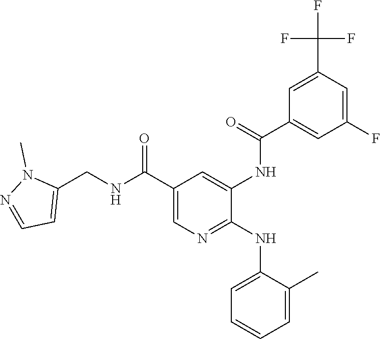 [SMILES: Cc1ccccc1Nc1ncc(C(=O)NCc2ccnn2C)cc1NC(=O)c1cc(F)cc(C(F)(F)F)c1] | (400 MHz, cdcl3) 8.52 (s, 1H), 8.43 (d, J = 2.1 Hz, 1H), 8.09 (d, J = 2.0 Hz, 1H), 7.75 (s, 1H), 7.63 (d, J = 8.4 Hz, 1H), 7.53-7.48 (m, 1H), 7.37 (d, J = 1.9 Hz, 1H), 7.33 (d, J = 7.9 Hz, 1H), 7.22 (d, J = 6.6 Hz, 1H), 7.14 (t, J = 7.0 Hz, 1H), 7.05 (td, J = 7.5, 1.1 Hz, 1H), 6.86 (s, 1H), 6.70 (t, J = 5.4 Hz, 1H), 6.18 (d, J = 1.9 Hz, 1H), 4.61 (d, J = 5.5 Hz, 2H), 3.81 (s, 3H), 2.25 (s, 3H). | 527.47 | B | B |
| I-53 | 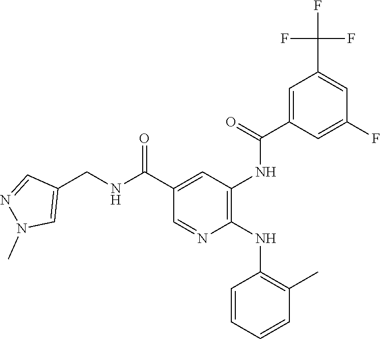 [SMILES: Cc1ccccc1Nc1ncc(C(=O)NCc2cnn(C)c2)cc1NC(=O)c1cc(F)cc(C(F)(F)F)c1] | (400 MHz, CDCl3) 8.49 (d, J = 2.1 Hz, 1H), 8.25 (m, 2H), 7.67 (s, 1H), 7.58 (d, J = 7.8 Hz, 1H), 7.50 (d, J = 7.8 Hz, 1H), 7.44 (s, 1H), 7.36 (s, 1H), 7.27 (d, J = 7.8 Hz, 1H), 7.25-7.22 (m, 1H), 7.15 (t, J = 7.8 Hz, 1H), 7.05 (dt, J = 7.8, 3.8 Hz, 1H), 6.83 (s, 1H), 6.30 (m, 1H), 4.44 (d, J = 5.5 Hz, 2H), 3.85 (s, 3H), 2.29 (s, 3H). | 527.45 | B | C |
| I-54 | 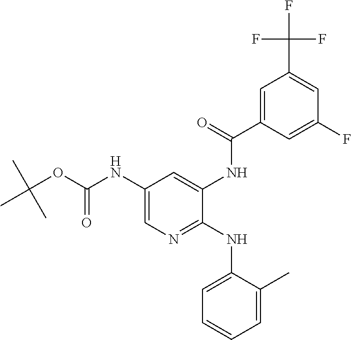 [SMILES: Cc1ccccc1Nc1ncc(NC(=O)OC(C)(C)C)cc1NC(=O)c1cc(F)cc(C(F)(F)F)c1] | (400 MHz, cdcl3) 8.64 (d, J = 2.5 Hz, 1H), 8.21 (d, J = 2.4 Hz, 1H), 7.75 (s, 1H), 7.43 (d, J = 7.9 Hz, 1H), 7.31 (d, J = 10.7 Hz, 2H), 7.27-7.23 (m, 1H (With CDCl3)), 7.07 (t, J = 6.9 Hz, 1H), 6.96 (td, J = 7.5, 1.2 Hz, 1H), 6.79 (d, J = 7.9 Hz, 1H), 6.53 (s, 1H), 6.07 (s, 1H), 2.34 (s, 3H), 1.58 (s, 9H). | 505.4 | D | D |
| I-55 | 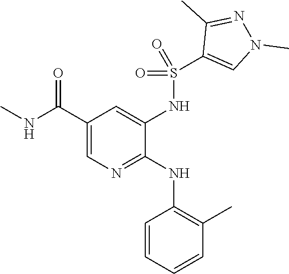 | (400 MHz, CD3OD) 8.38 (d, J = 2.2 Hz, 1H), 7.93 (s, 1H), 7.55 (d, J = 2.2 Hz, 1H), 7.52 (d, J = 7.5 Hz, 1H), 7.24 (d, J = 7.5 Hz, 1H), 7.18 (t, J = 7.5 Hz, 1H), 7.08 (t, J = 7.5 Hz, 1H), 3.79 (s, 3H), 2.85 (s, 3H), 2.20 (s, 3H), 2.15 (s, 3H). | 415.4 | E | |
| I-56 | 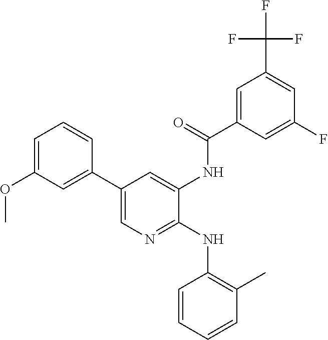 [SMILES: COc1cccc(-c2cnc(Nc3ccccc3C)c(NC(=O)c3cc(F)cc(C(F)(F)F)c3)c2)c1] | (400 MHz, dmso) 8.45 (d, J = 2.3 Hz, 1H), 8.37 (s, 1H), 8.30-8.23 (m, 2H), 8.22-8.12 (m, 3H), 7.97 (d, J = 8.4 Hz, 1H), 7.45 (d, J = 7.0 Hz, 1H), 7.29 (dd, J = 5.5, 1.5 Hz, 1H), 7.23 (d, J = 7.5 Hz, 1H), 7.18 (td, J = 7.8, 1.3 Hz, 1H), 7.08 (d, J = 0.9 Hz, 1H), 7.08-7.03 (m, 1H), 3.88 (s, 3H), 2.20 (s, 3H). | 497.4 | C | B |
| I-57 | 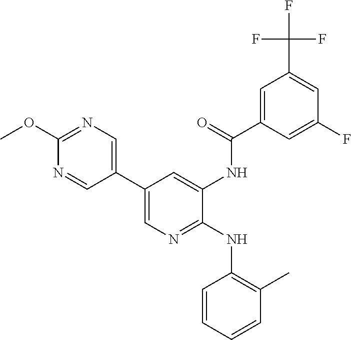 [SMILES: COc1ncc(-c2cnc(Nc3ccccc3C)c(NC(=O)c3cc(F)cc(C(F)(F)F)c3)c2)cn1] | (400 MHz, dmso) 10.39 (br s, 1H), 8.35 (d, J = 2.3 Hz, 1H), 8.25 (s, 1H), 8.18 (d, J = 9.1 Hz, 1H), 8.10-7.94 (m, 3H), 7.41 (d, J = 7.5 Hz, 1H), 7.22 (d, J = 7.3 Hz, 1H), 7.17 (td, J = 7.6, 1.4 Hz, 1H), 7.04 (td, J = 7.4, 1.1 Hz, 1H), 3.95 (s, 3H), 2.19 (s, 3H) | 498.43 | C | B |
| I-58 | 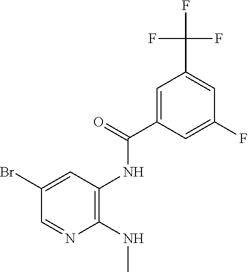 [SMILES: CNc1ncc(Br)cc1NC(=O)c1cc(F)cc(C(F)(F)F)c1] | | 392.1 | E | |
| I-59 | 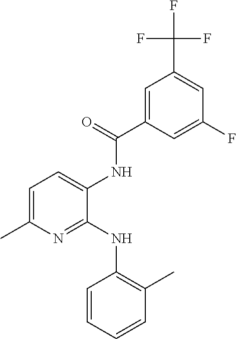 [SMILES: Cc1ccc(NC(=O)c2cc(F)cc(C(F)(F)F)c2)c(Nc2ccccc2C)n1] | | 404.4 | D | |
| I-60 | 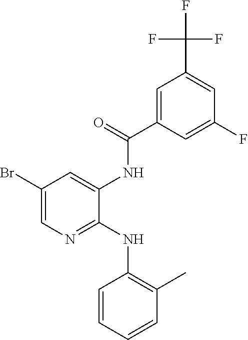 [SMILES: Cc1ccccc1Nc1ncc(Br)cc1NC(=O)c1cc(F)cc(C(F)(F)F)c1] | | 470.2 | D | B |
| I-61 | 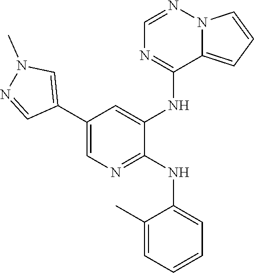 [SMILES: Cc1ccccc1Nc1ncc(-c2cnn(C)c2)cc1Nc1ncnn2cccc12] | | 397.4 | E | |
| I-62 | 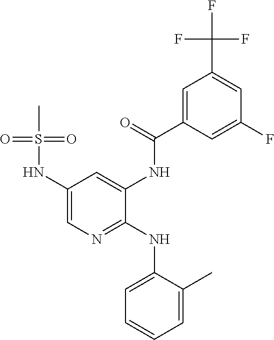 [SMILES: Cc1ccccc1Nc1ncc(NS(C)(=O)=O)cc1NC(=O)c1cc(F)cc(C(F)(F)F)c1] | (400 MHz, dmso) 10.32 (s, 1H), 9.46 (s, 1H), 8.22-8.12 (m, 2H), 7.98 (d, J = 8.5 Hz, 1H), 7.88 (d, J = 2.5 Hz, 1H), 7.76 (s, 1H), 7.58 (d, J = 2.5 Hz, 1H), 7.36 (d, J = 7.2 Hz, 1H), 7.22-7.09 (m, 2H), 6.98 (td, J = 7.4, 1.2 Hz, 1H), 2.96 (s, 3H), 2.15 (s, 3H). | 483.4 | D | |
| I-63 | 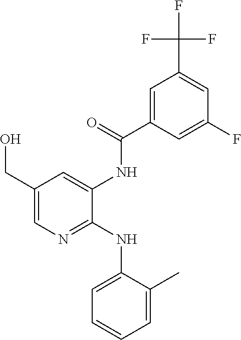 [SMILES: Cc1ccccc1Nc1ncc(CO)cc1NC(=O)c1cc(F)cc(C(F)(F)F)c1] | (400 MHz, dmso) 10.28 (s, 1H), 8.20 (s, 1H), 8.14 (d, J = 9.1 Hz, 1H), 7.98 (d, J = 8.5 Hz, 1H), 7.92 (d, J = 2.1 Hz, 1H), 7.68 (s, 1H), 7.60 (d, J = 2.1 Hz, 1H), 7.41 (d, J = 7.2 Hz, 1H), 7.17 (d, J = 7.4 Hz, 1H), 7.16-7.10 (m, 1H), 6.97 (td, J = 7.4, 1.2 Hz, 1H), 5.14 (t, J = 5.6 Hz, 1H), 4.41 (d, J = 5.6 Hz, 2H), 2.15 (s, 3H). | 420.4 | D | |
| I-64 | 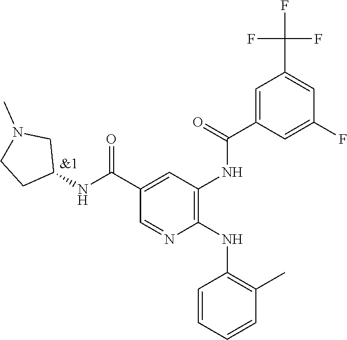 [SMILES: Cc1ccccc1Nc1ncc(C(=O)N[C@@H]2CCN(C)C2)cc1NC(=O)c1cc(F)cc(C(F)(F)F)c1] | (400 MHz, cdcl3) 9.16 (s, 1H), 8.67 (s, 1H), 8.52 (s, 1H), 8.31 (s, 1H), 8.20 (s, 1H), 7.94 (s, 1H), 7.81 (d, J = 8.4 Hz, 1H), 7.50 (d, J = 8.4 Hz, 1H), 7.45 (d, J = 7.8 Hz, 1H), 7.21 (d, J = 7.8 Hz, 1H), 7.18-7.11 (m, 2H), 7.02 (t, J = 7.8 Hz, 1H), 4.91 (m, 1H), 3.49 (m, 1H), 3.34 (d, J = 11.3 Hz, 1H), 2.83 (dd, J = 11.3, 6.8 Hz, 1H), 2.65 (s, 3H), 2.64-2.56 (m, 1H), 2.55-2.43 (m, 1H), 2.28 (s, 3H), 2.12-2.084 (m, 1H). | 516.47 | D | |
| I-65 | 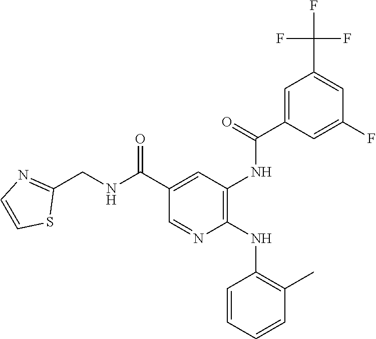 [SMILES: Cc1ccccc1Nc1ncc(C(=O)NCc2nccs2)cc1NC(=O)c1cc(F)cc(C(F)(F)F)c1] | (400 MHz, CDCl3) 8.59 (d, J = 2.2 Hz, 1H), 8.23 (d, J = 2.2 Hz, 1H), 8.19 (s, 1H), 7.73 (s, 1H), 7.71 (d, J = 3.5 Hz, 1H), 7.62 (d, J = 8.0 Hz, 1H), 7.57 (s, 1H), 7.51 (d, J = 8.0 Hz, 1H), 7.33 (d, J = 7.6 Hz, 1H), 7.28 (t, J = 3.5 Hz, 1H), 7.23 (d, J = 7.6 Hz, 1H), 7.12 (t, J = 7.6 Hz, 1H), 7.04 (t, J = 7.6 Hz, 1H), 6.94 (s, 1H), 4.95 (d, J = 5.6 Hz, 2H), 2.28 (s, 3H). | 530.39 | B | B |
| I-66 | 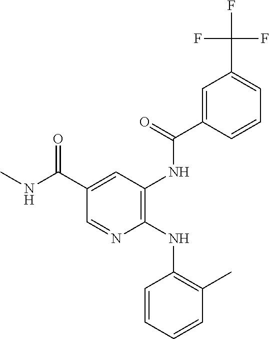 [SMILES: CNC(=O)c1cnc(Nc2ccccc2C)c(NC(=O)c2cccc(C(F)(F)F)c2)c1] | (400 MHz, CDCl3) 8.49 (s, 1H), 8.45 (d, J = 2.1 Hz, 1H), 8.20 (d, J = 2.1 Hz, 1H), 8.02 (s, 1H), 7.96 (d, J = 7.8 Hz, 1H), 7.81 (d, J = 7.8 Hz, 1H), 7.59 (t, J = 7.8 Hz, 1H), 7.38 (d, J = 7.6 Hz, 1H), 7.22 (d, J = 7.6 Hz, 1H), 7.15 (t, J = 7.6 Hz, 1H), 7.03 (t, J = 7.6 Hz, 1H), 6.98 (s,, 1H), 6.16 (d, J = 4.6 Hz, 1H), 2.94 (d, J = 4.6 Hz, 3H), 2.28 (s, 3H). | 429.4 | D | |
| I-67 | 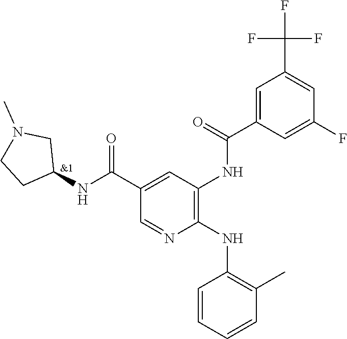 [SMILES: Cc1ccccc1Nc1ncc(C(=O)N[C@H]2CCN(C)C2)cc1NC(=O)c1cc(F)cc(C(F)(F)F)c1] | (400 MHz, cdcl3) 9.95 (s, 1H), 8.81 (d, J = 7.8 Hz, 1H), 8.62 (d, J = 2.1 Hz, 1H), 8.45 (s, 1H), 8.07 (s, 1H), 8.00 (d, J = 2.1 Hz, 1H), 7.90 (d, J = 7.8 Hz, 1H), 7.53 (d, J = 7.8 Hz, 1H), 7.46 (d, J = 7.8 Hz, 1H), 7.29 (s, 1H), 7.18-7.06 (m, 2H), 6.97 (t, J = 7.8 Hz, 1H), 4.87 (s, 1H), 3.67-3.53 (m, 1H), 3.47 (d, J = 11.7 Hz, 1H), 2.89 (dd, J = 11.7, 7.2 Hz, 1H), 2.75-2.70 (m, 1H), 2.68 (s, 3H), 2.52-2.40 (m, 1H), 2.21 (s, 3H), 2.18-2.05 (m, 1H). | 516.45 | C | D |
| I-68 | 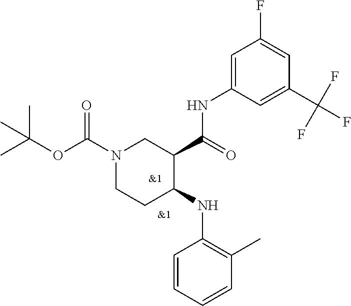 [SMILES: Cc1ccccc1N[C@H]1CCN(C(=O)OC(C)(C)C)C[C@H]1C(=O)Nc1cc(F)cc(C(F)(F)F)c1] | (400 MHz, dmso) 10.54 (s, 1H), 7.74 (d, J = 36.4 Hz, 2H), 7.32 (d, J = 8.4 Hz, 1H), 7.09-6.93 (m, 2H), 6.74 (s, 1H), 6.52 (t, J = 7.3 Hz, 1H), 4.32 (d, J = 8.7 Hz, 1H), 4.14 (s, 1H), 3.91 (d, J = 42.9 Hz, 2H), 3.44 (d, J = 12.6 Hz, 1H), 2.97 (d, J = 4.3 Hz, 2H), 2.19 (dd, J = 18.7, 9.3 Hz, 1H), 2.04-1.96 (m, 3H), 1.66 (s, 1H), 1.44-1.07 (m, 9H). | 496.5 | E | |
| I-69 | 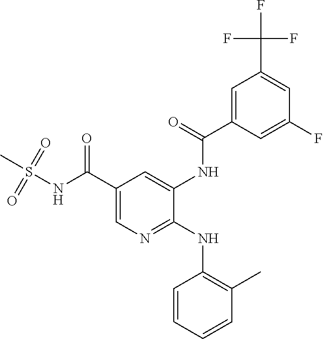 [SMILES: Cc1ccccc1Nc1ncc(C(=O)NS(C)(=O)=O)cc1NC(=O)c1cc(F)cc(C(F)(F)F)c1] | (400 MHz, cdcl3) 8.80 (s, 1H), 8.32 (s, 1H), 7.89 (s, 1H), 7.77 (d, J = 15.8 Hz, 2H), 7.45 (d, J = 7.2 Hz, 1H), 7.35 (s, 1H), 6.99 (m, 4H), 3.19 (s, 3H), 2.10 (s, 3H). | 511.37 | E | |
| I-70 | 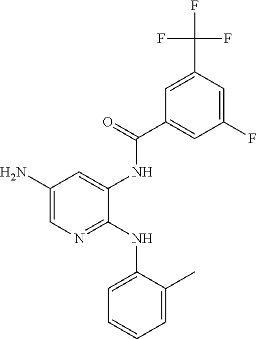 [SMILES: Cc1ccccc1Nc1ncc(N)cc1NC(=O)c1cc(F)cc(C(F)(F)F)c1] | (400 MHz, dmso) 10.20 (s, 1H), 8.07 (s, 1H), 8.02 (d, J = 8.9 Hz, 1H), 7.96 (d, J = 8.5 Hz, 1H), 7.59 (d, J = 2.6 Hz, 1H), 7.26-7.21 (m, 1H), 7.17 (d, J = 2.6 Hz, 1H), 7.06 (d, J = 7.3 Hz, 1H), 7.03- 6.95 (m, 1H), 6.92 (s, 1H), 6.72 (td, J = 7.4, 1.2 Hz, 1H), 5.02 (bs, 2H), 2.13 (s, 3H). | 405.4 | D | |
| I-71 | 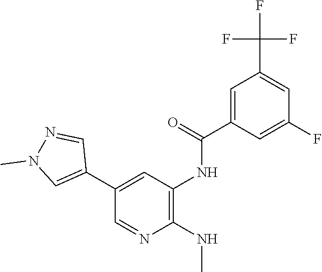 [SMILES: CNc1ncc(-c2cnn(C)c2)cc1NC(=O)c1cc(F)cc(C(F)(F)F)c1] | 9.98-10.02 (m, 1H), 8.19-8.27 (m, 2H), 8.15 (br d, J = 9.3 Hz, 1H), 7.93-8.02 (m, 2H), 7.73 (s, 1H), 7.61 (d, J = 2.0 Hz, 1H), 6.29 (br d, J = 4.6 Hz, 1H), 3.83 (s, 3H), 2.83 (d, J = 4.4 Hz, 3H) | 394.3 | E | |
| I-72 | 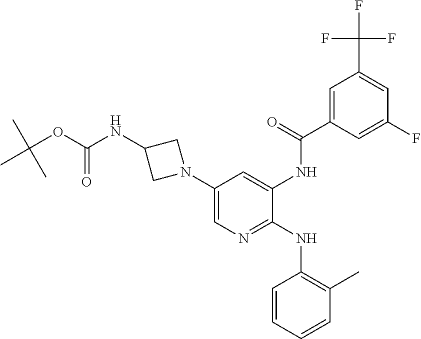 [SMILES: Cc1ccccc1Nc1ncc(N2CC(NC(=O)OC(C)(C)C)C2)cc1NC(=O)c1cc(F)cc(C(F)(F)F)c1] | 10.29 (s, 1H), 8.12 (s, 1H), 7.94-8.08 (m, 2H), 7.29-7.58 (m, 3H), 7.06-7.22 (m, 2H), 6.93-7.05 (m, 2H), 6.78 (brt, J = 7.3 Hz, 1H), 6.73-6.73 (m, 1H), 6.64 (d, J = 8.5 Hz, 1H), 4.33-4.48 (m, 1H), 4.07 (br t, J = 7.2 Hz, 1H), 3.55 (br t, J = 6.7 Hz, 1H), 2.13 (s, 2H), 1.16-1.41 (m, 8H) | 506.7 | D | |
| I-73 | 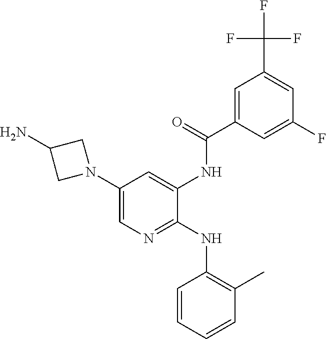 [SMILES: Cc1ccccc1Nc1ncc(N2CC(N)C2)cc1NC(=O)c1cc(F)cc(C(F)(F)F)c1] | | 460.5 | D | |
| I-74 | 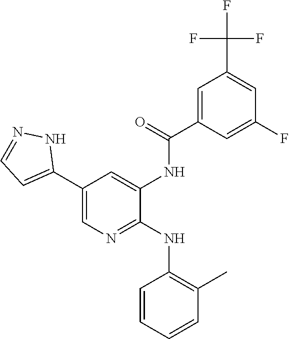 [SMILES: Cc1ccccc1Nc1ncc(-c2ccn[nH]2)cc1NC(=O)c1cc(F)cc(C(F)(F)F)c1] | | | D | |
| I-75 | 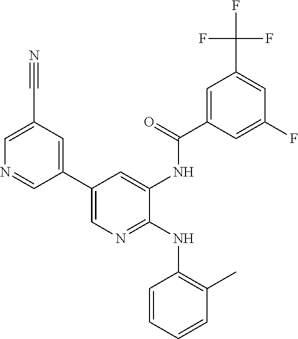 [SMILES: Cc1ccccc1Nc1ncc(-c2cncc(C#N)c2)cc1NC(=O)c1cc(F)cc(C(F)(F)F)c1] | | | B | B |
| I-76 | 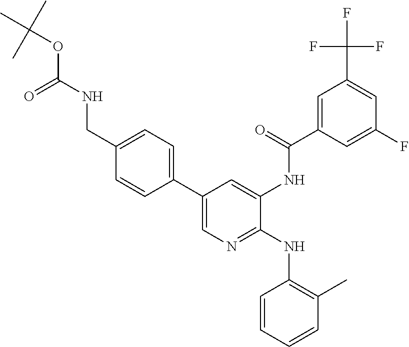 [SMILES: Cc1ccccc1Nc1ncc(-c2ccc(CNC(=O)OC(C)(C)C)cc2)cc1NC(=O)c1cc(F)cc(C(F)(F)F)c1] | | | D | |
| I-77 | 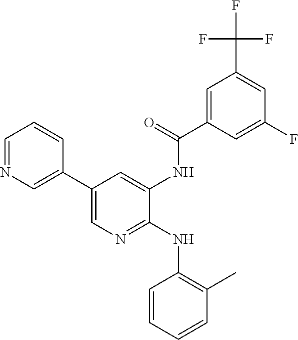 [SMILES: Cc1ccccc1Nc1ncc(-c2cccnc2)cc1NC(=O)c1cc(F)cc(C(F)(F)F)c1] | | | D | |
| I-78 | 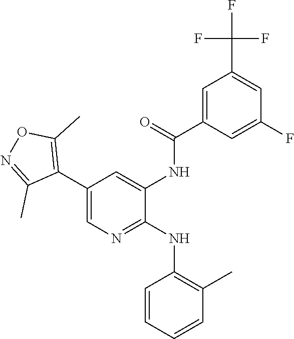 [SMILES: Cc1ccccc1Nc1ncc(-c2c(C)noc2C)cc1NC(=O)c1cc(F)cc(C(F)(F)F)c1] | | | C | |
| I-79 | 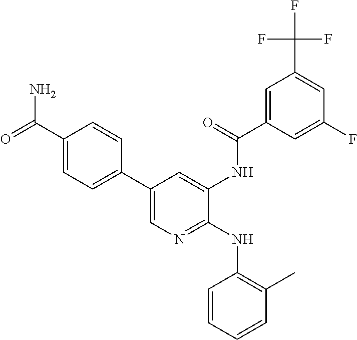 [SMILES: Cc1ccccc1Nc1ncc(-c2ccc(C(N)=O)cc2)cc1NC(=O)c1cc(F)cc(C(F)(F)F)c1] | | | B | C |
| I-80 | 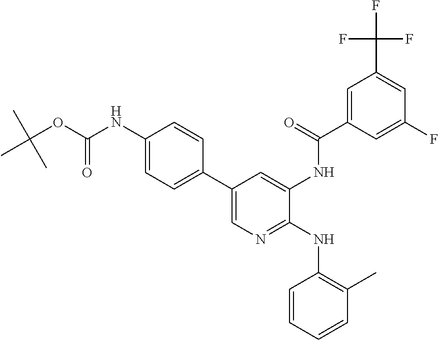 [SMILES: Cc1ccccc1Nc1ncc(-c2ccc(NC(=O)OC(C)(C)C)cc2)cc1NC(=O)c1cc(F)cc(C(F)(F)F)c1] | | | D | |
| I-81 | 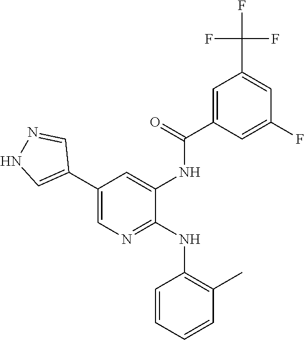 [SMILES: Cc1ccccc1Nc1ncc(-c2cn[nH]c2)cc1NC(=O)c1cc(F)cc(C(F)(F)F)c1] | | | D | |
| I-82 | 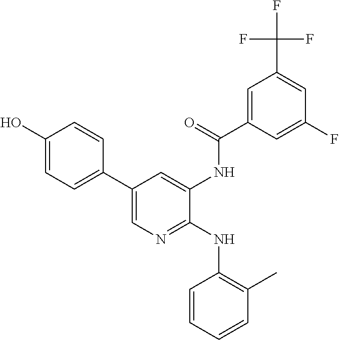 [SMILES: Cc1ccccc1Nc1ncc(-c2ccc(O)cc2)cc1NC(=O)c1cc(F)cc(C(F)(F)F)c1] | | | C | D |
| I-83 | 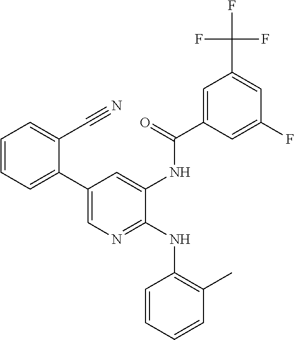 [SMILES: Cc1ccccc1Nc1ncc(-c2ccccc2C#N)cc1NC(=O)c1cc(F)cc(C(F)(F)F)c1] | | | C | |
| I-84 | 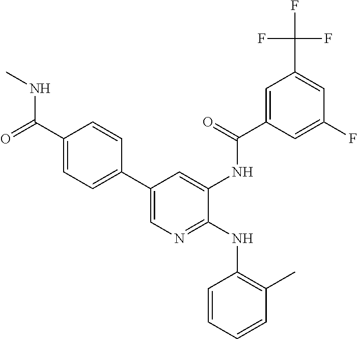 [SMILES: CNC(=O)c1ccc(-c2cnc(Nc3ccccc3C)c(NC(=O)c3cc(F)cc(C(F)(F)F)c3)c2)cc1] | | | D | C |
| I-85 | 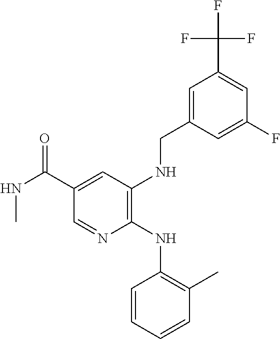 [SMILES: CNC(=O)c1cnc(Nc2ccccc2C)c(NCc2cc(F)cc(C(F)(F)F)c2)c1] | (400 MHz, CDCl3) 8.06-8.00 (m, 1H), 7.40 (s, 1H), 7.37 (d, J = 2.0 Hz, 1H), 7.27-7.16 (m, 5H), 7.06 (td, J = 7.6, 1.6 Hz 1H), 6.10 (m, 1H), 4.43 (s, 2H), 2.97 (d, J = 4.8 Hz, 3H), 2.29 (s, 3H). | 433.4 | E | |
| I-86 | 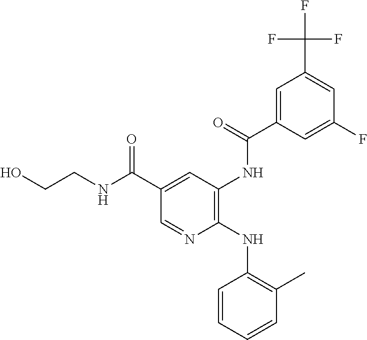 [SMILES: Cc1ccccc1Nc1ncc(C(=O)NCCO)cc1NC(=O)c1cc(F)cc(C(F)(F)F)c1] | (400 MHz, CD3OD) 8.44 (d, J = 2.2 Hz, 1H), 8.19 (s, 1H), 8.05 (d, J = 2.2 Hz, 1H), 8.02 (s, 1H), 7.73 (d, J = 8.2 Hz, 1H), 7.30 (dd, J = 7.8, 1.4 Hz, 1H), 7.24 (d, J = 7.8 Hz, 1H), 7.17 (dt, J = 7.8, 3.9 Hz, 1H), 7.11 (td, J = 7.8, 1.4 Hz, 1H), 4.60 (s, 1H), 3.69 (t, J = 5.8 Hz, 2H), 3.48 (t, J = 5.8 Hz, 2H), 2.22 (s, 3H). | 477.39 | C | |
| I-87 | 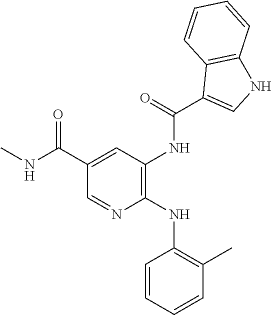 [SMILES: CNC(=O)c1cnc(Nc2ccccc2C)c(NC(=O)c2c[nH]c3ccccc23)c1] | (400 MHz, CD3OD) 8.42 (d, J = 2.2 Hz, 1H), 8.22-8.18 (m, 1H), 8.13 (s, 1H), 8.06 (d, J = 2.2 Hz, 1H), 7.53 (dd, J = 7.6, 1.6 Hz, 1H), 7.49-7.43 (m, 1H), 7.25-7.15 (m, 4H), 7.05 (td, J = 7.6, 1.6 Hz, 1H), 2.90 (s, 3H), 2.24 (s, 3H). | 400.41 | E | |
| I-88 | 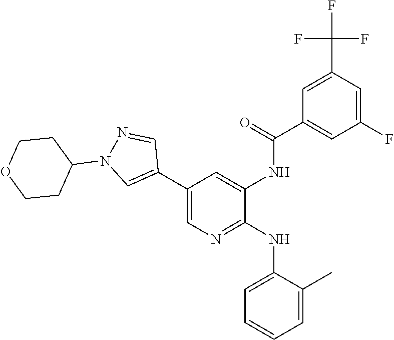 [SMILES: Cc1ccccc1Nc1ncc(-c2cnn(C3CCOCC3)c2)cc1NC(=O)c1cc(F)cc(C(F)(F)F)c1] | (DMSO-d6, 400 MHz): = 10.35 (s, 1H), 8.29 (br d, J = 1.5 Hz, 1H), 8.23 (br s, 1H), 8.22 (br s, 1H), 8.16 (br d, J = 9.0 Hz, 1H), 7.99 (br d, J = 8.1 Hz, 1H), 7.85 (br s, 2H), 7.73 (s, 1H), 7.45 (br d, J = 7.8 Hz, 1H), 7.07-7.21 (m, 2H), 6.98 (br t, J = 7.3 Hz, 1H), 4.34- 4.48 (m, 1H), 3.96 (br d, J = 10.7 Hz, 2H), 3.42-3.54 (m, 2H), 2.18 (s, 3H), 1.89-2.06 ppm (m, 4H). | 540.7 | C | |
| I-89 | 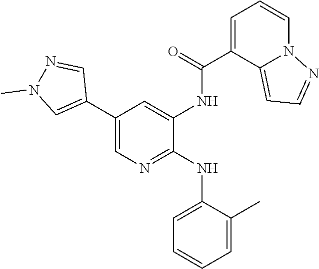 [SMILES: Cc1ccccc1Nc1ncc(-c2cnn(C)c2)cc1NC(=O)c1cccn2nccc12] | (DMSO-d6, 400 MHz): = 10.22 (s, 1H), 8.92 (d, J = 6.8 Hz, 1H), 8.28 (d, J = 2.2 Hz, 1H), 8.13 (d, J = 2.2 Hz, 1H), 8.10 (s, 1H), 7.96 (d, J = 6.8 Hz, 1H), 7.90 (d, J = 2.0 Hz, 1H), 7.83 (s, 1H), 7.62 (s, 1H), 7.56 (br d, J = 7.8 Hz, 1H), 7.10-7.20 (m, 2H), 7.06 (t, J = 7.1 Hz, 1H), 7.03 (d, J = 2.0 Hz, 1H), 6.94 (t, J = 7.3 Hz, 1H), 3.86 (s, 3H), 2.17 ppm (s, 3H). | 424.4 | E | |
| I-90 | 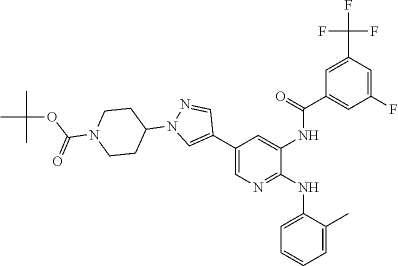 [SMILES: Cc1ccccc1Nc1ncc(-c2cnn(C3CCN(C(=O)OC(C)(C)C)CC3)c2)cc1NC(=O)c1cc(F)cc(C(F)(F)F)c1] | (DMSO-d6, 400 MHz): = 10.33 (s, 1H), 8.28 (d, J = 2.0 Hz, 1H), 8.23 (s, 2H), 8.16 (br d, J = 9.0 Hz, 1H), 7.99 (br d, J = 8.3 Hz, 1H), 7.84 (s, 2H), 7.73 (br s, 1H), 7.45 (br d, J = 7.6 Hz, 1H), 7.19 (br d, J = 7.6 Hz, 1H), 7.14 (br t, J = 7.6 Hz, 1H), 6.95-7.01 (m, 1H), 4.36 (tt, J = 11.3, 3.8 Hz, 1H), 4.04 (br d, J = 12.0 Hz, 2H), 2.17 (s, 3H), 1.98-2.08 (m, 2H), 1.79 (qd, J = 12.1, 4.3 Hz, 2H), 1.42 (s, 9H), 0.81- 0.90 ppm (m, 2H) | 639.6 | D | |
| I-91 | 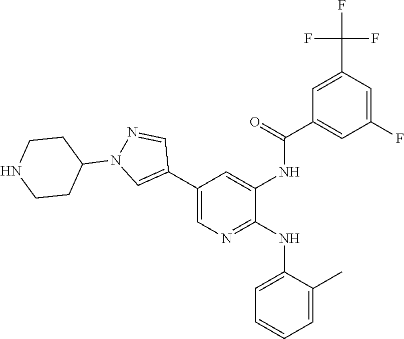 [SMILES: Cc1ccccc1Nc1ncc(-c2cnn(C3CCNCC3)c2)cc1NC(=O)c1cc(F)cc(C(F)(F)F)c1] | (DMSO-d6, 400 MHz): = 10.26 (br s, 1H), 8.27 (d, J = 2.0 Hz, 1H), 8.23 (s, 1H), 8.17 (s, 1H), 8.15 (br s, 1H), 7.98 (br d, J = 8.5 Hz, 1H), 7.88 (br s, 1H), 7.82 (s, 1H), 7.76 (br s, 1H), 7.49 (br d, J = 7.8 Hz, 1H), 7.18 (d, J = 7.3 Hz, 1H), 7.14 (br t, J = 7.7 Hz, 1H), 6.97 (t, J = 7.2 Hz, 1H), 4.20 (tt, J = 11.4, 3.9 Hz, 1H), 3.06 (br d, J = 12.5 Hz, 2H), 2.57-2.69 (m, 2H), 2.18 (s, 3H), 1.95- 2.04 (m, 2H), 1.80 (qd, J = 12.0, 3.9 Hz, 2H), 1.07 ppm (s, 1H) | 539.6 | C | |
| I-92 | 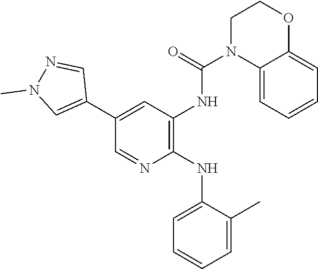 [SMILES: Cc1ccccc1Nc1ncc(-c2cnn(C)c2)cc1NC(=O)N1CCOc2ccccc21] | (DMSO-d6, 400 MHz): = 8.64 (br s, 1H), 8.20 (br s, 1H), 8.07 (s, 1H), 7.76- 7.86 (m, 2H), 7.71 (br d, J = 8.1 Hz, 1H), 7.49-7.58 (m, J = 2.7 Hz, 2H), 7.17 (br d, J = 7.3 Hz, 1H), 7.12 (br t, J = 7.6 Hz, 1H), 6.82-6.99 (m, 4H), 4.26- 4.33 (m, 2H), 3.88-3.96 (m, 2H), 3.86 (s, 3H), 2.17 ppm (s, 3H) | 441.5 | E | |
| I-93 | 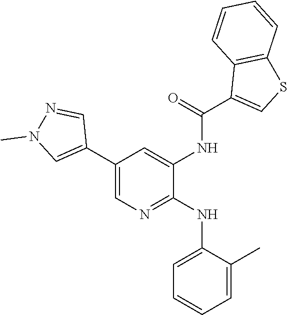 [SMILES: Cc1ccccc1Nc1ncc(-c2cnn(C)c2)cc1NC(=O)c1csc2ccccc12] | (DMSO-d6, 400 MHz): = 10.16 (s, 1H), 8.68 (s, 1H), 8.50 (br d, J = 7.6 Hz, 1H), 8.29 (d, J = 2.2 Hz, 1H), 8.11 (s, 1H), 8.10 (s, 1H), 7.92 (d, J = 2.0 Hz, 1H), 7.83 (s, 1H), 7.66 (s, 1H), 7.61 (br d, J = 7.8 Hz, 1H), 7.44-7.53 (m, 2H), 7.11-7.20 (m, 2H), 6.94 (t, J = 7.3 Hz, 1H), 3.87 (s, 3H), 2.19 ppm (s, 3H) | 440.4 | B | C |
| I-94 | 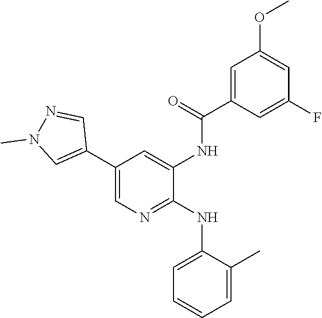 [SMILES: COc1cc(F)cc(C(=O)Nc2cc(-c3cnn(C)c3)cnc2Nc2ccccc2C)c1] | (DMSO-d6, 400 MHz): = 10.15 (s, 1H), 8.27 (d, J = 2.0 Hz, 1H), 8.09 (s, 1H), 7.84 (br s, 1H), 7.82 (s, 1H), 7.60 (br s, 1H), 7.56 (br d, J = 8.1 Hz, 1H), 7.45 (s, 1H), 7.42 (br d, J = 9.0 Hz, 1H), 7.06-7.20 (m, 3H), 6.92-6.97 (m, 1H), 3.86 (s, 6H), 2.17 ppm (s, 3H) | 432.5 | E | |
| I-95 | 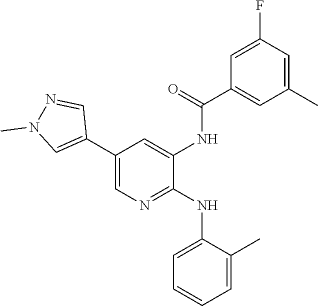 | | 416.5 | D | |
| I-96 | 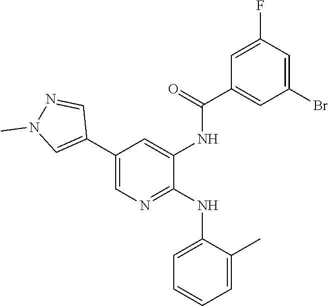 [SMILES: Cc1ccccc1Nc1ncc(-c2cnn(C)c2)cc1NC(=O)c1cc(F)cc(Br)c1] | | 480.4 | C | |
| I-97 | 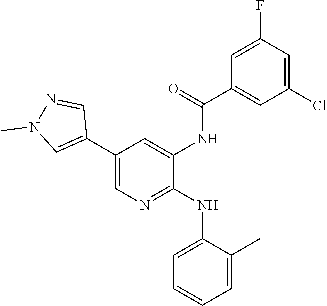 [SMILES: Cc1ccccc1Nc1ncc(-c2cnn(C)c2)cc1NC(=O)c1cc(F)cc(Cl)c1] | | 436.4 | C | |
| I-98 | 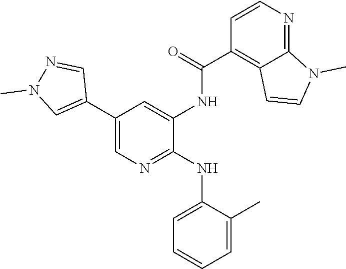 | | 438.4 | E | |
| I-99 | 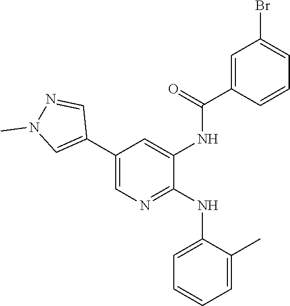 [SMILES: Cc1ccccc1Nc1ncc(-c2cnn(C)c2)cc1NC(=O)c1cccc(Br)c1] | | 462.3 | D | |
| I- 100 | 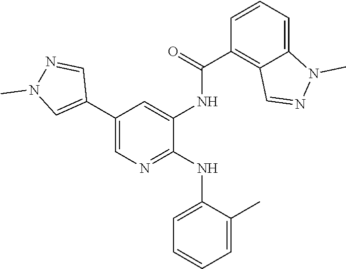 | | 438.5 | E | |
| I- 101 | 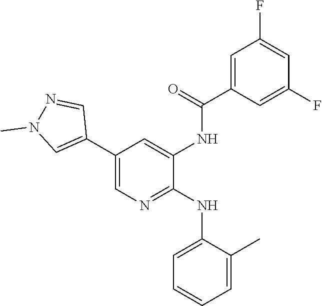 [SMILES: Cc1ccccc1Nc1ncc(-c2cnn(C)c2)cc1NC(=O)c1cc(F)cc(F)c1] | | 420.4 | D | |
| I- 102 | 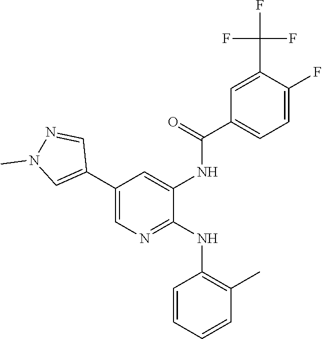 [SMILES: Cc1ccccc1Nc1ncc(-c2cnn(C)c2)cc1NC(=O)c1ccc(F)c(C(F)(F)F)c1] | | | D | |
| I- 103 | 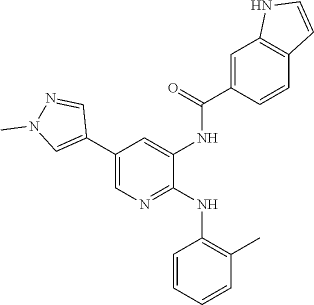 [SMILES: Cc1ccccc1Nc1ncc(-c2cnn(C)c2)cc1NC(=O)c1ccc2cc[nH]c2c1] | | | E | |
| I- 104 | 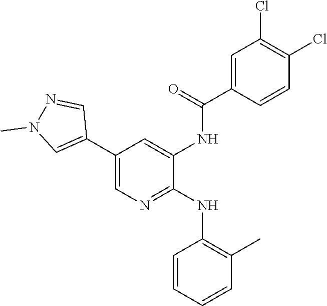 [SMILES: Cc1ccccc1Nc1ncc(-c2cnn(C)c2)cc1NC(=O)c1ccc(Cl)c(Cl)c1] | | | D | |
| I- 105 | 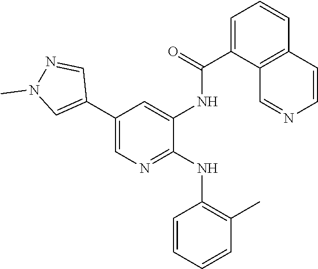 [SMILES: Cc1ccccc1Nc1ncc(-c2cnn(C)c2)cc1NC(=O)c1cccc2ccncc12] | | | E | |
| I- 106 | 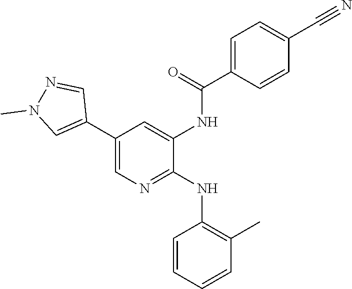 [SMILES: Cc1ccccc1Nc1ncc(-c2cnn(C)c2)cc1NC(=O)c1ccc(C#N)cc1] | | | E | |
| I- 107 | 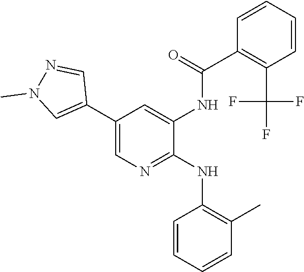 [SMILES: Cc1ccccc1Nc1ncc(-c2cnn(C)c2)cc1NC(=O)c1ccccc1C(F)(F)F] | | | E | |
| I- 108 | 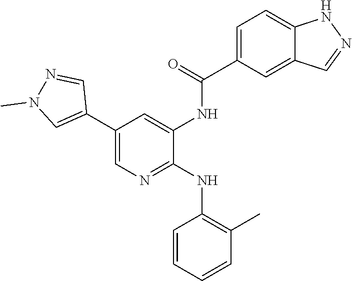 [SMILES: Cc1ccccc1Nc1ncc(-c2cnn(C)c2)cc1NC(=O)c1ccc2[nH]ncc2c1] | | | E | |
| I- 109 | 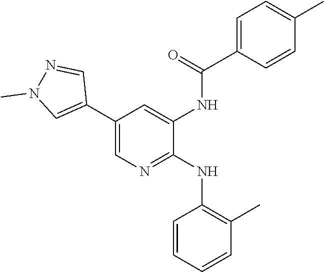 | | | E | |
| I- 110 | 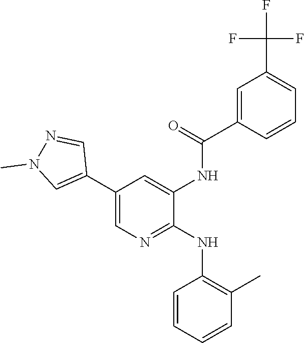 [SMILES: Cc1ccccc1Nc1ncc(-c2cnn(C)c2)cc1NC(=O)c1cccc(C(F)(F)F)c1] | | | D | |
| I- 111 | 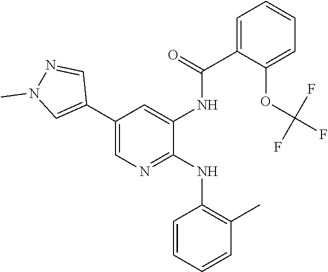 [SMILES: Cc1ccccc1Nc1ncc(-c2cnn(C)c2)cc1NC(=O)c1ccccc1OC(F)(F)F] | | | E | |
| I- 112 | 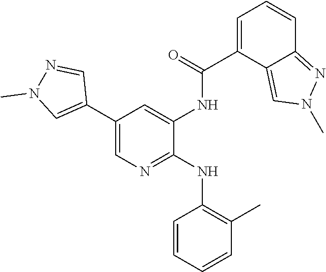 | | | E | |
| I- 113 | 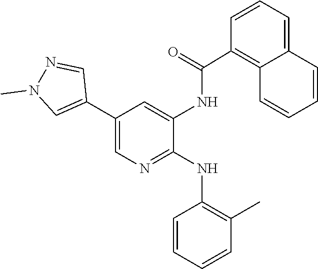 [SMILES: Cc1ccccc1Nc1ncc(-c2cnn(C)c2)cc1NC(=O)c1cccc2ccccc12] | | | E | |
| I- 114 | 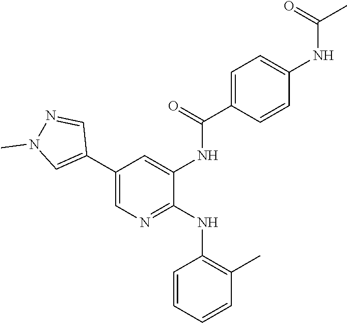 [SMILES: CC(=O)Nc1ccc(C(=O)Nc2cc(-c3cnn(C)c3)cnc2Nc2ccccc2C)cc1] | | | E | |
| I- 115 | 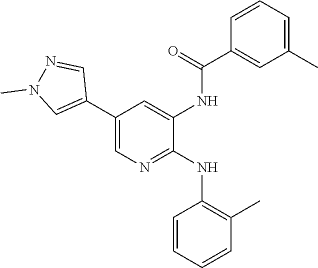 | | | E | |
| I- 116 | 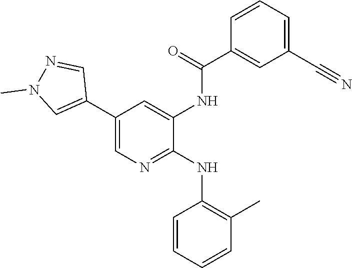 [SMILES: Cc1ccccc1Nc1ncc(-c2cnn(C)c2)cc1NC(=O)c1cccc(C#N)c1] | | | E | |
| I- 117 | 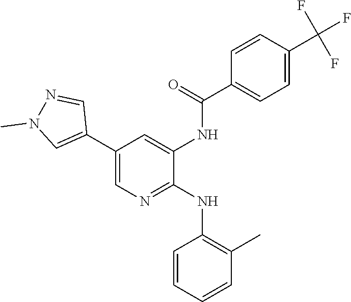 [SMILES: Cc1ccccc1Nc1ncc(-c2cnn(C)c2)cc1NC(=O)c1ccc(C(F)(F)F)cc1] | | | E | |
| I- 118 | 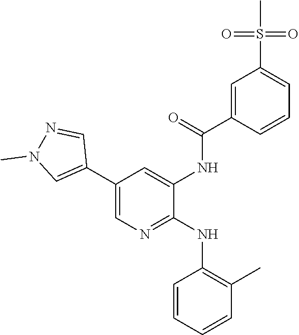 [SMILES: Cc1ccccc1Nc1ncc(-c2cnn(C)c2)cc1NC(=O)c1cccc(S(C)(=O)=O)c1] | | | E | |
| I- 119 | 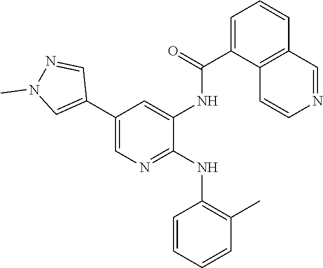 [SMILES: Cc1ccccc1Nc1ncc(-c2cnn(C)c2)cc1NC(=O)c1cccc2cnccc12] | | | E | |
| I- 120 | 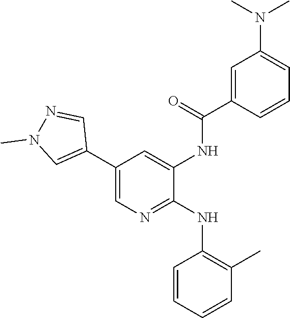 | | | E | |
| I- 121 | 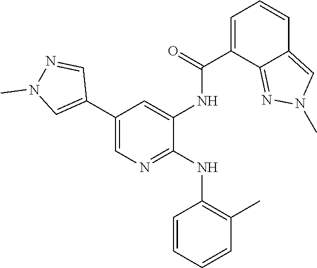 | | | E | |
| I- 122 | 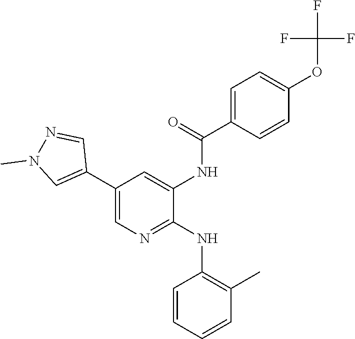 [SMILES: Cc1ccccc1Nc1ncc(-c2cnn(C)c2)cc1NC(=O)c1ccc(OC(F)(F)F)cc1] | | | E | |
| I- 123 | 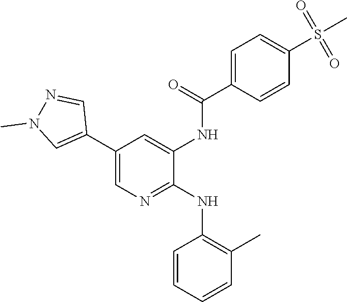 | | | E | |
| I- 124 | 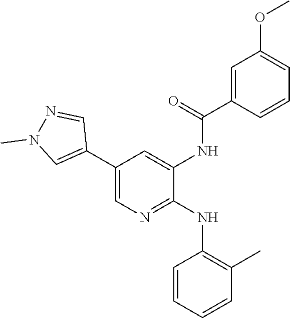 | | | E | |
| I- 125 | 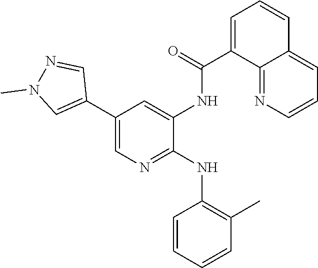 [SMILES: Cc1ccccc1Nc1ncc(-c2cnn(C)c2)cc1NC(=O)c1cccc2cccnc12] | | | E | |
| I- 126 | 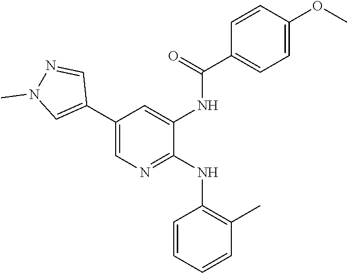 | | | E | |
| I- 127 | 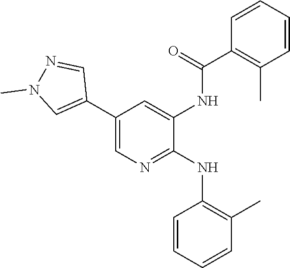 [SMILES: Cc1ccccc1Nc1ncc(-c2cnn(C)c2)cc1NC(=O)c1ccccc1C] | | | E | |
| I- 128 | 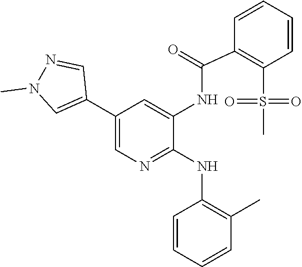 [SMILES: Cc1ccccc1Nc1ncc(-c2cnn(C)c2)cc1NC(=O)c1ccccc1S(C)(=O)=O] | | | E | |
| I- 129 | 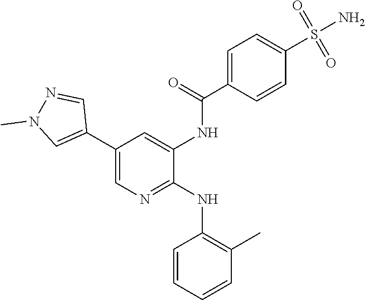 [SMILES: Cc1ccccc1Nc1ncc(-c2cnn(C)c2)cc1NC(=O)c1ccc(S(N)(=O)=O)cc1] | | | E | |
| I- 130 | 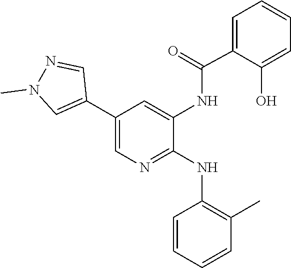 [SMILES: Cc1ccccc1Nc1ncc(-c2cnn(C)c2)cc1NC(=O)c1ccccc1O] | | | E | |
| I- 131 | 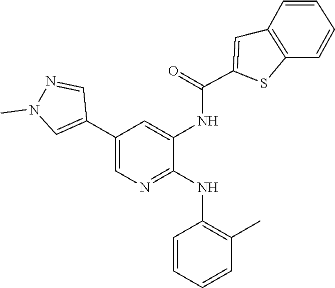 [SMILES: Cc1ccccc1Nc1ncc(-c2cnn(C)c2)cc1NC(=O)c1cc2ccccc2s1] | | 440.4 | E | |
| I- 132 | 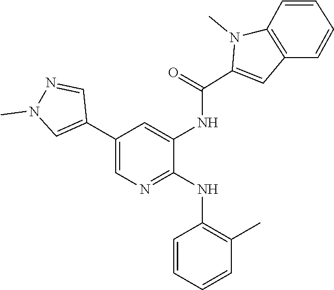 [SMILES: Cc1ccccc1Nc1ncc(-c2cnn(C)c2)cc1NC(=O)c1cc2ccccc2n1C] | | 437.5 | D | |
| I- 133 | 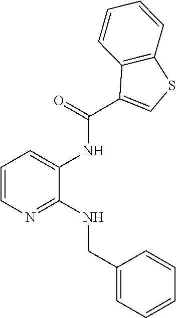 [SMILES: O=C(Nc1cccnc1NCc1ccccc1)c1csc2ccccc12] | | 360.3 | E | |
| I- 134 | 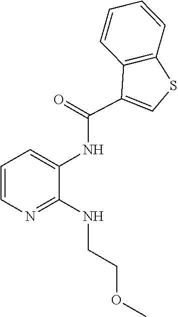 [SMILES: COCCNc1ncccc1NC(=O)c1csc2ccccc12] | | 328.3 | E | |
| I- 135 | 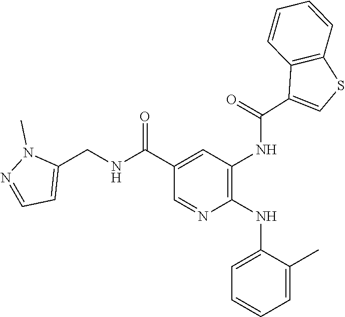 [SMILES: Cc1ccccc1Nc1ncc(C(=O)NCc2ccnn2C)cc1NC(=O)c1csc2ccccc12] | (500 MHz, DMSO-d6) Shift 10.04 (s, 1H), 8.83 (t, J = 5.64 Hz, 1H), 8.69 (s, 1H), 8.45-8.50 (m, 2H), 8.27 (br s, 1H), 8.15 (d, J = 2.14 Hz, 1H), 8.08 (d, J = 7.63 Hz, 1H), 7.39-7.50 (m, 3H), 7.29 (d, J = 1.53 Hz, 1H), 7.22 (d, J = 7.63 Hz, 1H), 7.17 (t, J = 7.63 Hz, 1H), 7.03-7.09 (m, 1H), 6.15 (d, J = 1.53 Hz, 1H), 4.51 (d, J = 5.80 Hz, 2H), 3.81 (s, 3H), 2.17 (s, 3H) | 497.3 | B | B |
| I- 136 | 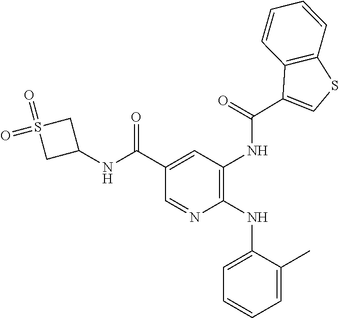 [SMILES: Cc1ccccc1Nc1ncc(C(=O)NC2CS(=O)(=O)C2)cc1NC(=O)c1csc2ccccc12] | (500 MHz, DMSO-d6) Shift 10.04 (s, 1H), 8.92 (br d, J = 3.05 Hz, 1H), 8.70 (s, 1H), 8.49 (d, J = 7.93 Hz, 1H), 8.47 (d, J = 2.14 Hz, 1H), 8.33 (s, 1H), 8.13 (d, J = 2.14 Hz, 1H), 8.08 (d, J = 7.93 Hz, 1H), 7.47-7.53 (m, 1H), 7.42-7.47 (m, 1H), 7.41 (d, J = 7.93 Hz, 1H), 7.22 (d, J = 7.32 Hz, 1H), 7.18 (t, J = 7.47 Hz, 1H), 7.02-7.10 (m, 1H), 4.52-4.59 (m, 3H), 4.23-4.31 (m, 2H), 2.16 (s, 3H) | 507.3 | A | B |
| I- 137 | 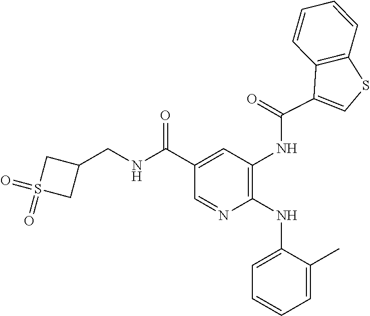 [SMILES: Cc1ccccc1Nc1ncc(C(=O)NCC2CS(=O)(=O)C2)cc1NC(=O)c1csc2ccccc12] | (500 MHz, DMSO-d6) 10.04 (s, 1H), 8.69 (s, 1H), 8.62 (t, J = 5.80 Hz, 1H), 8.48 (d, J = 7.63 Hz, 1H), 8.44 (d, J = 2.14 Hz, 1H), 8.25 (s, 1H), 8.10 (d, J = 2.14 Hz, 1H), 8.08 (d, J = 7.63 Hz, 1H), 7.44-7.53 (m, 2H), 7.38-7.44 (m, 1H), 7.22 (d, J = 7.32 Hz, 1H), 7.17 (t, J = 7.47 Hz, 1H), 7.02-7.11 (m, 1H), 4.25 (dd, J = 9.76, 14.03 Hz, 2H), 3.95 (br dd, J = 5.49, 14.34 Hz, 2H), 3.50 (t, J = 6.41 Hz, 2H), 2.72-2.81 (m, 1H), 2.16 (s, 3H) | 521.3 | B | C |
| I- 138 | 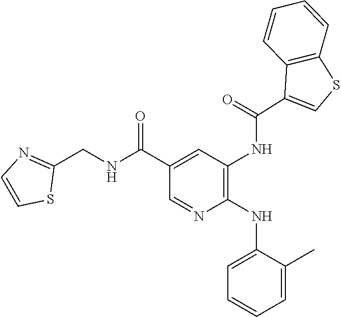 [SMILES: Cc1ccccc1Nc1ncc(C(=O)NCc2nccs2)cc1NC(=O)c1csc2ccccc12] | (500 MHz, DMSO-d6) 10.05 (s, 1H), 9.27 (t, J = 5.80 Hz, 1H), 8.70 (s, 1H), 8.44-8.53 (m, 2H), 8.27-8.36 (m, 1H), 8.17 (d, J = 1.83 Hz, 1H), 8.08 (d, J = 7.93 Hz, 1H), 7.72 (d, J = 3.05 Hz, 1H), 7.62 (d, J = 3.36 Hz, 1H), 7.38-7.53 (m, 3H), 7.23 (d, J = 7.63 Hz, 1H), 7.18 (t, J = 7.47 Hz, 1H), 7.04-7.11 (m, 1H), 4.74 (d, J = 5.80 Hz, 2H), 2.17 (s, 3H) | 500.3 | A | C |
| I- 139 | 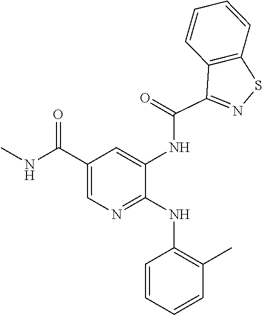 [SMILES: CNC(=O)c1cnc(Nc2ccccc2C)c(NC(=O)c2nsc3ccccc23)c1] | (400 MHz, cdcl3) 9.12 (s, 1H), 9.00- 8.93 (m, 1H), 8.52 (d, J = 2.2 Hz, 1H), 8.26 (d, J = 2.2 Hz, 1H), 8.06-7.95 (m, 1H), 7.65-7.55 (m, 2H), 7.52 (d, J = 7.6 Hz, 1H), 7.22-7.16 (m, 2H), 7.09 (s, 1H), 7.03 (td, J = 7.6, 1.2 Hz, 1H), 6.06 (m, 1H), 3.01 (d, J = 4.8 Hz, 3H), 2.31 (s, 3H). | 418.37 | C | D |
| I- 140 | 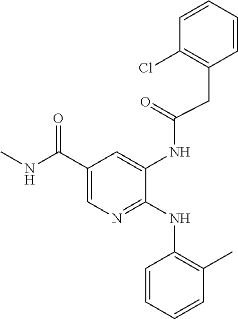 [SMILES: CNC(=O)c1cnc(Nc2ccccc2C)c(NC(=O)Cc2ccccc2Cl)c1] | (400 MHz, cdcl3) 8.44 (d, J = 2.1 Hz, 1H), 8.01 (d, J = 2.1 Hz, 1H), 7.47-7.39 (m, 1H), 7.34-7.25 (m, 4H), 7.20 (d, J = 7.2 Hz, 2H), 7.16 (td, J = 7.2, 1.2 Hz, 1H), 7.03 (td, J = 7.2, 1.2 Hz, 1H), 6.99 (s, 1H), 6.76 (s, 1H), 6.01 (m, 1H), 3.85 (s, 2H), 2.97 (t, J = 4.8 Hz, 3H), 2.19 (s, 3H). | 409.38 | E | |
| I- 141 | 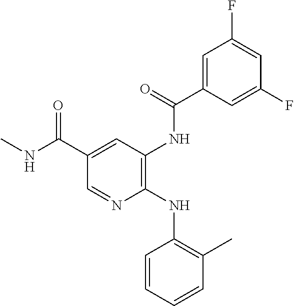 [SMILES: CNC(=O)c1cnc(Nc2ccccc2C)c(NC(=O)c2cc(F)cc(F)c2)c1] | (400 MHz, cdcl3) 8.44 (d, J = 2.1 Hz, 2H), 8.20 (d, J = 2.1 Hz, 1H), 7.35 (d, J = 8.0 Hz, 1H), 7.26 (m, 2H), 7.22 (m, 1H), 7.17 (td, J = 8.0, 1.6 Hz, 1H), 7.05 (td, J = 8.0, 1.6 Hz, 1H), 6.98 (tt, J = 8.0, 1.6 Hz, 1H), 6.90 (s, 1H), 6.14 (q, J = 4.8 Hz, 1H), 2.96 (d, J = 4.8 Hz, 3H), 2.27 (s, 3H). | 397.37 | D | |
| I- 142 | 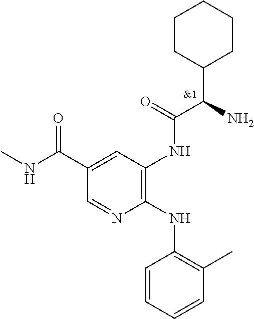 [SMILES: CNC(=O)c1cnc(Nc2ccccc2C)c(NC(=O)[C@H](N)C2CCCCC2)c1] | (400 MHz, cd3od) 8.37 (d, J = 2.2 Hz, 1H), 7.99 (d, J = 2.2 Hz, 1H), 7.32 (dd, J = 7.4, 1.2 Hz, 1H), 7.26 (d, J = 7.4 Hz, 1H), 7.20 (t, J = 7.4 Hz, 1H), 7.13 (td, J = 7.4, 1.2 Hz, 1H), 3.63 (d, J = 5.9 Hz, 1H), 2.89 (s, 3H), 2.22 (s, 3H), 1.90-1.82 (m, 5H), 1.74 (d, J = 12.6 Hz, 1H), 1.40-1.15 (m, 5H). | 396.46 | E | |
| I- 143 | 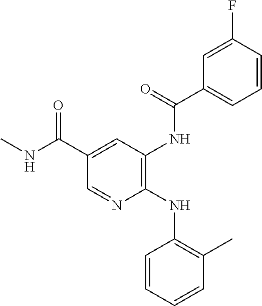 [SMILES: CNC(=O)c1cnc(Nc2ccccc2C)c(NC(=O)c2cccc(F)c2)c1] | (400 MHz, CDCl3) 8.46 (s, 1H), 8.26 (s, 1H), 8.20 (s, 1H), 7.53-7.37 (m, 4H), 7.28-7.21 (m, 2H), 7.17 (t, J = 7.6 Hz, 1H), 7.04 (t, J = 7.6 Hz, 1H), 6.97 (s, 1H), 6.15 (s, 1H), 2.96 (d, J = 4.8 Hz, 3H), 2.28 (s, 3H). | 379.39 | E | |
| I- 144 | 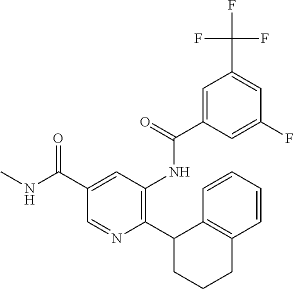 [SMILES: CNC(=O)c1cnc(C2CCCc3ccccc32)c(NC(=O)c2cc(F)cc(C(F)(F)F)c2)c1] | (400 MHz, DMSO-d6) 9.98 (s, 1H), 8.75 (d, J = 2.2 Hz, 1H), 8.56 (q, J = 4.5 Hz, 1H), 8.19 (d, J = 2.2 Hz, 1H), 7.84 (d, J = 8.5 Hz, 1H), 7.38-7.33 (d, J = 8.5 Hz, 1H), 7.32 (s, 1H), 6.92 (d, J = 7.5 Hz, 1H), 6.85-6.76 (m, 1H), 6.63 (td, J = 7.4, 1.1 Hz, 1H), 6.39-6.30 (m, 1H), 3.85 (t, J = 6.2 Hz, 2H), 2.82 (d, J = 4.5 Hz, 3H), 2.72-2.66 (m, 2H), 2.00 (p, J = 6.4 Hz, 2H). Contains 3% weight DMF. | 473.37 | D | E |
| I- 145 | 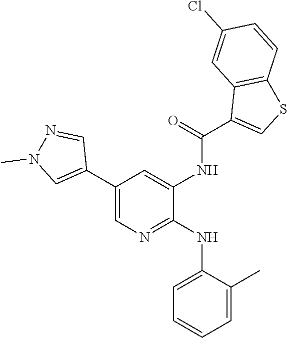 [SMILES: Cc1ccccc1Nc1ncc(-c2cnn(C)c2)cc1NC(=O)c1csc2ccc(Cl)cc12] | | 474.4 | D | |
| I- 146 | 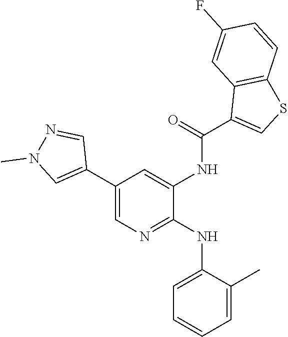 [SMILES: Cc1ccccc1Nc1ncc(-c2cnn(C)c2)cc1NC(=O)c1csc2ccc(F)cc12] | | 458.6 | B | B |
| I- 147 | 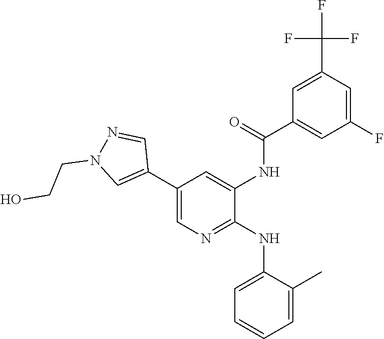 [SMILES: Cc1ccccc1Nc1ncc(-c2cnn(CCO)c2)cc1NC(=O)c1cc(F)cc(C(F)(F)F)c1] | | 500.6 | C | |
| I- 148 | 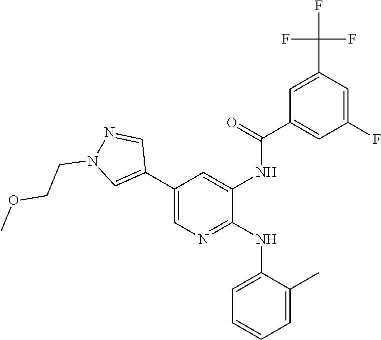 [SMILES: COCCn1cc(-c2cnc(Nc3ccccc3C)c(NC(=O)c3cc(F)cc(C(F)(F)F)c3)c2)cn1] | | 514.5 | C | |
| I- 149 | 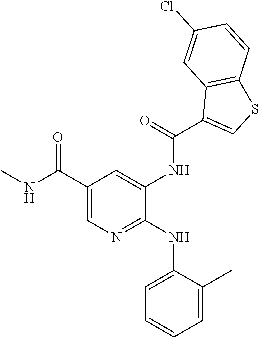 [SMILES: CNC(=O)c1cnc(Nc2ccccc2C)c(NC(=O)c2csc3ccc(Cl)cc23)c1] | | 451.4 | B | D |
| I- 150 | 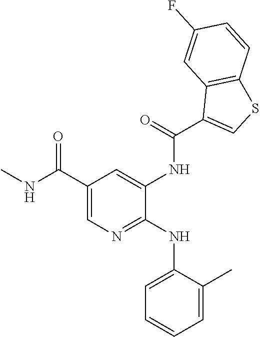 [SMILES: CNC(=O)c1cnc(Nc2ccccc2C)c(NC(=O)c2csc3ccc(F)cc23)c1] | | 435.4 | B | C |
| I- 151 | 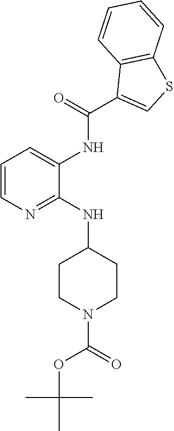 [SMILES: CC(C)(C)OC(=O)N1CCC(Nc2ncccc2NC(=O)c2csc3ccccc23)CC1] | | 453.4 | E | |
| I- 152 | 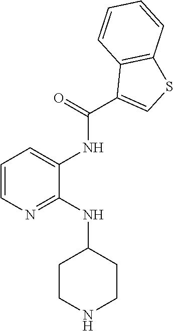 [SMILES: O=C(Nc1cccnc1NC1CCNCC1)c1csc2ccccc12] | | 353.3 | E | |
| I- 153 | 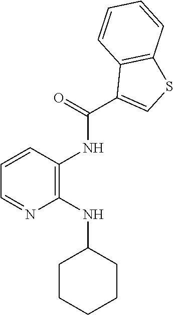 [SMILES: O=C(Nc1cccnc1NC1CCCCC1)c1csc2ccccc12] | | 352.3 | E | |
| I- 154 | 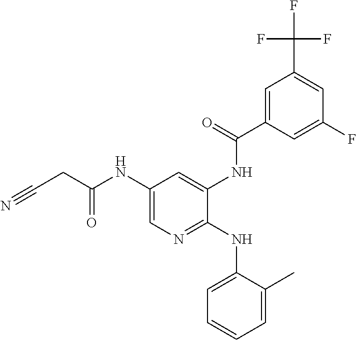 [SMILES: Cc1ccccc1Nc1ncc(NC(=O)CC#N)cc1NC(=O)c1cc(F)cc(C(F)(F)F)c1] | 10.34 (s, 1H), 8.20 (s, 1H), 8.14 (d, J = 9.1 Hz, 1H), 8.10 (d, J = 2.4 Hz, 1H), 8.01 (s, 1H), 7.97 (d, J = 8.1 Hz, 1H), 7.76 (s, 1H), 7.42 (s, 1H), 7.17 (d, J = 7.5 Hz, 1H), 7.12 (td, J = 7.6, 1.2 Hz, 1H), 6.96 (td, J = 7.4, 1.0 Hz, 1H), 3.89 (s, 2H), 2.15 (s, 3H). | 472.4 | D | |
| I- 155 | 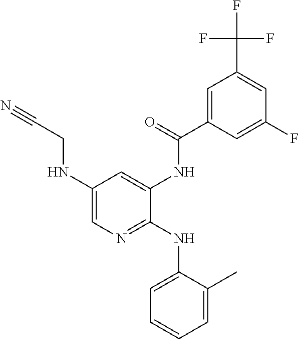 [SMILES: Cc1ccccc1Nc1ncc(NCC#N)cc1NC(=O)c1cc(F)cc(C(F)(F)F)c1] | 10.36 (s, 1H), 8.43 (residual AmF, partially salt), 8.15 (s, 1H), 8.08 (d, J = 9.6 Hz, 1H), 7.96 (d, J = 7.8Hz, 1H), 7.68 (s, 1H), 7.32 (m, 2H), 7.20 (bs, 1H), 7.10 (d, J = 7.5 Hz, 1H), 7.05 (t, J = 7.7 Hz, 1H), 6.81 (t, J = 7.9 Hz, 1H), 6.11 (s, 1H), 4.28 (d, J = 7.0 Hz, 2H), 2.15 (s, 3H) | 444.4 | C | |
| I- 156 | 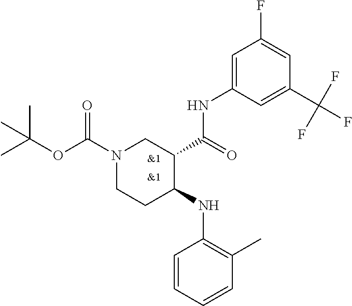 [SMILES: Cc1ccccc1N[C@H]1CCN(C(=O)OC(C)(C)C)C[C@@H]1C(=O)Nc1cc(F)cc(C(F)(F)F)c1] | 10.52 (s, 1H), 7.71 (s, 1H), 7.66 (dd, J = 11.0, 1.9 Hz, 1H), 7.32 (d, J = 8.6 Hz, 1H), 6.95 (t, J = 7.7 Hz, 1H), 6.88 (d, J = 7.3 Hz, 1H), 6.67 (d, J = 8.1 Hz, 1H), 6.45 (t, J = 7.3 Hz, 1H), 4.32 (d, J = 9.6 Hz, 1H), 4.16 (s, 1H), 3.97 (d, J = 11.8 Hz, 1H), 3.81 (d, J = 6.7 Hz, 1H), 2.95 (bs, 2H), 2.61 (td, J = 10.8, 4.0 Hz, 1H), 2.09-2.06 (s, 3H, ACN), 2.06- 2.02 (m, 1H), 2.00 (s, 3H), 1.44 (d, J = 20.3 Hz, 9H), 1.26 (qd, J = 12.9, 4.9 Hz, 1H). | 496.5 | E | |
| I- 157 | 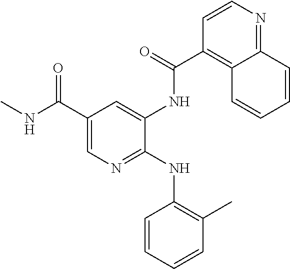 [SMILES: CNC(=O)c1cnc(Nc2ccccc2C)c(NC(=O)c2ccnc3ccccc23)c1] | (400 MHz, cdcl3) 8.92 (d, J = 4.3 Hz, 1H), 8.49 (d, J = 2.2 Hz, 1H), 8.37 (d, J = 2.2 Hz, 1H), 8.29 (d, J = 8.2 Hz, 1H), 8.17 (d, J = 8.2 Hz, 1H), 8.08 (s, 1H), 7.80 (t, J = 7.8 Hz, 1H), 7.64 (dd, J = 7.8, 4.3 Hz, 1H), 7.37 (d, J = 8.2 Hz, 1H), 7.24-7.18 (m, 3H), 7.06 (t, J = 8.2 Hz, 2H), 6.09 (m, 1H), 2.90 (d, J = 4.8 Hz, 3H), 2.30 (s, 3H). Addition, there is impurity peak at 1.25 ppm. | 412.42 | E | |
| I- 158 | 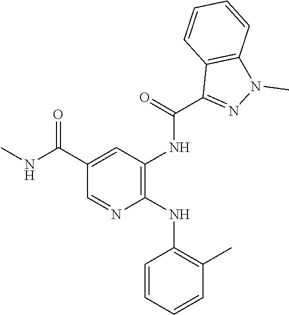 | (400 MHz, cdcl3) 8.70 (s, 1H), 8.50 (d, J = 2.2 Hz, 1H), 8.35 (dt, J = 8.0, 1.2 Hz, 1H), 8.23 (d, J = 2.2 Hz, 1H), 7.55 (d, J = 8.0 Hz, 1H), 7.51-7.42 (m, 2H), 7.36-7.32 (m, 1H), 7.31-7.27 (m, 1H), 7.23-7.15 (m, 2H), 7.03 (dt, J = 8.0, 1.2 Hz, 1H), 6.08 (m, 1H), 4.12 (s, 3H), 3.00 (d, J = 4.8 Hz, 3H), 2.31 (s, 3H). <10 mol % impurities. | 415.43 | E | |
| I- 159 | 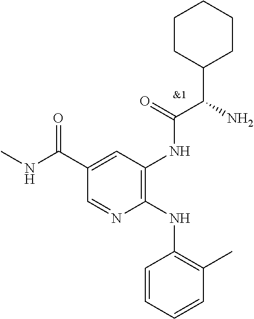 [SMILES: CNC(=O)c1cnc(Nc2ccccc2C)c(NC(=O)[C@@H](N)C2CCCCC2)c1] | (400 MHz, CD3OD) 8.37 (d, J = 2.2 Hz, 1H), 8.00 (d, J = 2.2 Hz, 1H), 7.32 (dd, J = 7.6, 1.4 Hz, 1H), 7.26 (d, J = 7.6 Hz, 1H), 7.19 (t, J = 7.6 Hz, 1H), 7.12 (td, J = 7.6, 1.4 Hz, 1H), 3.68 (d, J = 6.1 Hz, 1H), 2.89 (s, 3H), 2.22 (s, 3H), 1.96-1.84 (m, 5H), 1.74 (d, J = 12.6 Hz, 1H), 1.38-1.16 (m, 5H). | 396.49 | E | |
| I- 160 | 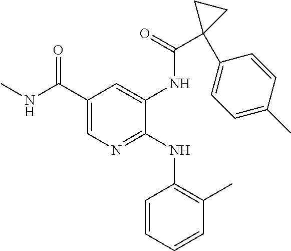 | | | E | |
| I- 161 | 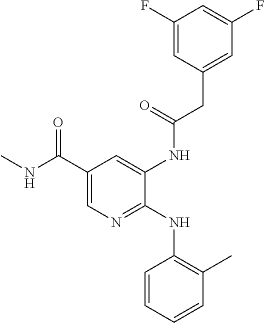 [SMILES: CNC(=O)c1cnc(Nc2ccccc2C)c(NC(=O)Cc2cc(F)cc(F)c2)c1] | | | E | |
| I- 162 | 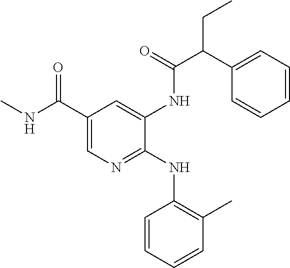 [SMILES: CCC(C(=O)Nc1cc(C(=O)NC)cnc1Nc1ccccc1C)c1ccccc1] | | | E | |
| I- 163 | 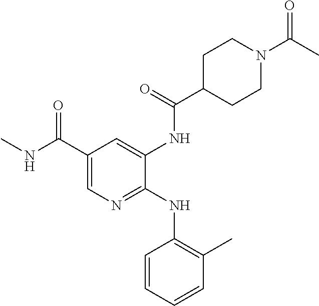 [SMILES: CNC(=O)c1cnc(Nc2ccccc2C)c(NC(=O)C2CCN(C(C)=O)CC2)c1] | | | E | |
| I- 164 | 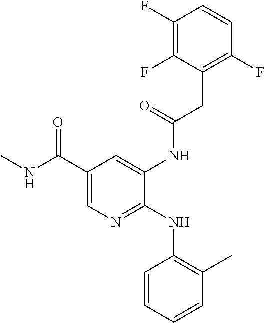 [SMILES: CNC(=O)c1cnc(Nc2ccccc2C)c(NC(=O)Cc2c(F)ccc(F)c2F)c1] | | | E | |
| I- 165 | 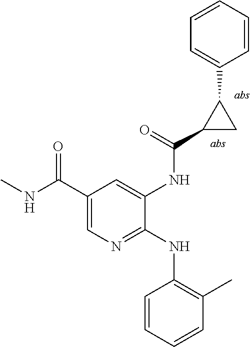 [SMILES: CNC(=O)c1cnc(Nc2ccccc2C)c(NC(=O)[C@@H]2C[C@H]2c2ccccc2)c1] | | | E | |
| I- 166 | 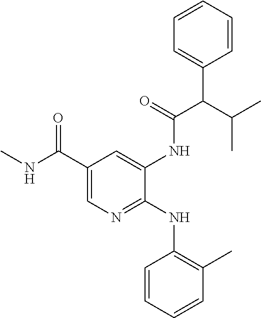 [SMILES: CNC(=O)c1cnc(Nc2ccccc2C)c(NC(=O)C(c2ccccc2)C(C)C)c1] | | | E | |
| I- 167 | 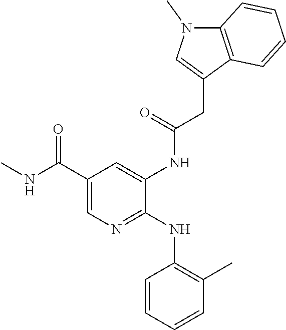 [SMILES: CNC(=O)c1cnc(Nc2ccccc2C)c(NC(=O)Cc2cn(C)c3ccccc23)c1] | | | E | |
| I- 168 | 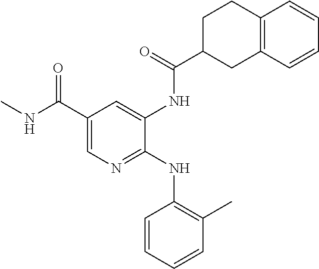 [SMILES: CNC(=O)c1cnc(Nc2ccccc2C)c(N(C)C(=O)C2CCc3ccccc3C2)c1] | | | E | |
| I- 169 | 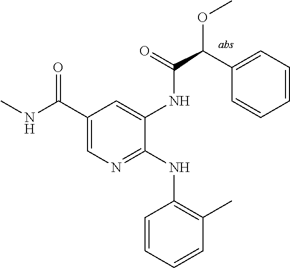 [SMILES: CNC(=O)c1cnc(Nc2ccccc2C)c(NC(=O)[C@@H](OC)c2ccccc2)c1] | | | E | |
| I- 170 | 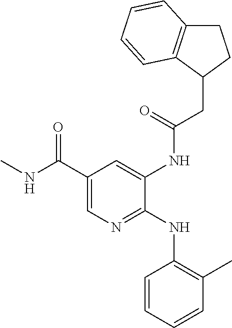 | | | E | |
| I- 171 | 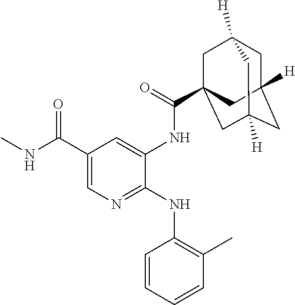 [SMILES: CNC(=O)c1cnc(Nc2ccccc2C)c(NC(=O)[C@]23C[C@H]4C[C@H](C[C@H](C4)C2)C3)c1] | | | D | |
| I- 172 | 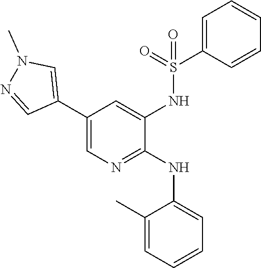 [SMILES: Cc1ccccc1Nc1ncc(-c2cnn(C)c2)cc1NS(=O)(=O)c1ccccc1] | (DMSO-d6, 400 MHz) 9.88 (s, 1H), 8.16 (br s, 1H), 7.91 (s, 1H), 7.73 (br d, J = 7.8 Hz, 2H), 7.60-7.66 (m, 1H), 7.49-7.59 (m, 4H), 7.46 (br s, 1H), 7.25 (d, J = 1.2 Hz, 1H), 7.14 (br d, J = 7.3 Hz, 1H), 7.09 (br t, J = 7.7 Hz, 1H), 6.92 (br t, J = 7.3 Hz, 1H), 3.83 (s, 3H), 2.02 ppm (s, 3H). | 420.4 | E | |
| I- 173 | 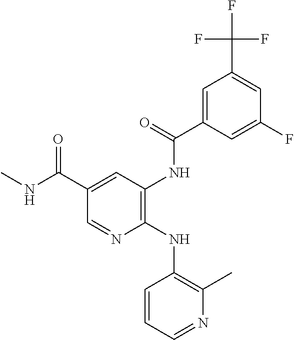 [SMILES: CNC(=O)c1cnc(Nc2cccnc2C)c(NC(=O)c2cc(F)cc(C(F)(F)F)c2)c1] | (400 MHz, dmso) 10.33 (s, 1H), 8.40 (d, J = 2.1 Hz, 1H), 8.37 (s, 1H), 8.29 (q, J = 4.4 Hz, 1H), 8.24-8.18 (m, 2H), 8.15 (d, J = 9.0 Hz, 1H), 8.00 (s, 1H), 7.97 (d, J = 8.4 Hz, 1H), 7.65 (d, J = 7.8 Hz, 1H), 7.20 (dd, J = 8.0, 4.8 Hz, 1H), 2.73 (d, J = 4.5 Hz, 3H), 2.32 (s, 3H). | 448.4 | E | |
| I- 174 | 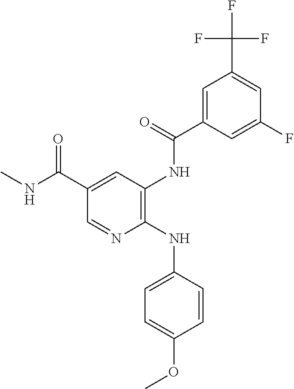 [SMILES: CNC(=O)c1cnc(Nc2ccc(OC)cc2)c(NC(=O)c2cc(F)cc(C(F)(F)F)c2)c1] | (400 MHz, dmso) 10.28 (s, 0.5H), 8.59 (s, 1H), 8.49 (d, J = 2.2 Hz, 1H), 8.42 (s, 0.5H), 8.30 (br d, J = 4.7 Hz, 1H), 8.26 (s, 1H), 8.19 (d, J = 9.2 Hz, 1H), 8.00 (s, 1H and d, J = 9.2 Hz, 1H), 7.51 (d, J = 8.0 Hz, 2H), 6.90 (d, J = 8.0 Hz, 2H), 3.73 (s, 3H), 2.77 (d, J = 4.5 Hz, 3H). | 463.39 | E | |
| I- 175 | 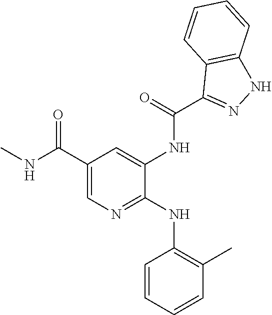 [SMILES: CNC(=O)c1cnc(Nc2ccccc2C)c(NC(=O)c2n[nH]c3ccccc23)c1] | (400 MHz, cdcl3) 8.82 (s, 1H), 8.48 (d, J = 2.2 Hz, 1H), 8.36 (d, J = 8.2 Hz, 1H), 8.20 (d, J = 2.2 Hz, 1H), 7.56 (d, J = 4.0 Hz, 1H), 7.54 (d, J = 4.0 Hz, 1H), 7.50-7.42 (m, 1H), 7.35 (s, 1H), 7.32 (dd, J = 7.5, 1.0 Hz, 1H), 7.20-7.14 (m, 2H), 7.01 (td, J = 7.5, 1.0 Hz, 1H), 6.15 (q, J = 4.8 Hz, 1H), 3.01 (d, J = 4.8 Hz, 3H), 2.28 (s, 3H). | 401.42 | E | |
| I- 176 | 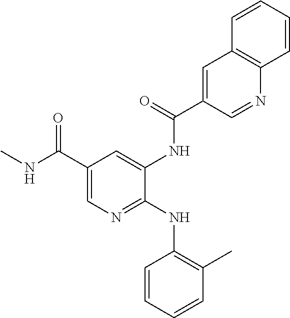 [SMILES: CNC(=O)c1cnc(Nc2ccccc2C)c(NC(=O)c2cnc3ccccc3c2)c1] | (400 MHz, cdcl3) 9.32 (d, J = 2.2 Hz, 1H), 8.99 (s, 1H), 8.50 (d, J = 2.1 Hz, 1H), 8.45 (d, J = 2.2 Hz, 1H), 8.22 (d, J = 2.1 Hz, 1H), 8.13 (d, J = 8.4 Hz, 1H), 7.85-7.79 (m, 2H), 7.61 (ddd, J = 8.4, 7.4, 1.1 Hz, 1H), 7.42 (dd, J = 7.4, 1.2 Hz, 1H), 7.21 (d, J = 7.4 Hz, 1H), 7.14 (td, J = 7.4, 1.2 Hz, 2H), 7.02 (td, J = 7.4, 1.2 Hz, 1H), 6.36 (q, J = 4.8 Hz, 1H), 2.91 (d, J = 4.8 Hz, 3H), 2.27 (s, 3H). | 412.43 | E | |
| I- 177 | 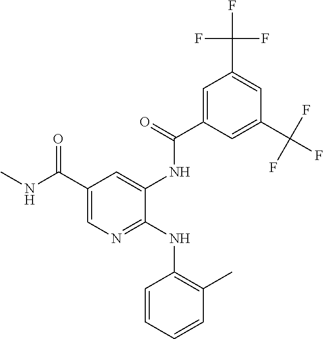 [SMILES: CNC(=O)c1cnc(Nc2ccccc2C)c(NC(=O)c2cc(C(F)(F)F)cc(C(F)(F)F)c2)c1] | (400 MHz, cdcl3) 9.07 (s, 1H), 8.40 (d, J = 2.2 Hz, 1H), 8.30 (s, 2H), 8.17 (d, J = 2.2 Hz, 1H), 8.04 (s, 1H), 7.36 (d, J = 7.8 Hz, 1H), 7.21 (d, J = 7.8 Hz, 1H), 7.14 (td, J = 7.8, 1.2 Hz, 1H), 7.02 (td, J = 7.8, 1.2 Hz, 1H), 6.90 (s, 1H), 6.19 (q, J = 4.8 Hz, 1H), 2.93 (d, J = 4.8 Hz, 3H), 2.27 (s, 3H). | 497.4 | E | |
| I- 178 | 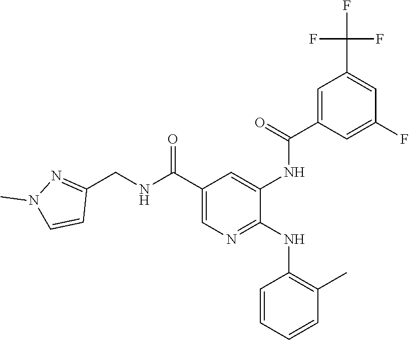 [SMILES: Cc1ccccc1Nc1ncc(C(=O)NCc2ccn(C)n2)cc1NC(=O)c1cc(F)cc(C(F)(F)F)c1] | (400 MHz, cdcl3) 8.54 (d, J = 2.2 Hz, 2H), 8.05 (d, J = 2.2 Hz, 1H), 7.89 (s, 1H), 7.76 (d, J = 7.8 Hz, 1H), 7.64 (m, 1H), 7.52 (d, J = 7.8 Hz, 1H), 7.36 (d, J = 7.8 Hz, 1H), 7.24 (m, 1H), 7.20 (d, J = 7.8 Hz, 1H), 7.10 (td, J = 7.8, 1.4 Hz, 1H), 7.04 (s, 1H), 7.01 (td, J = 7.8, 1.4 Hz, 1H), 6.16 (d, J = 2.4 Hz, 1H), 4.65 (d, J = 4.8 Hz, 2H), 3.82 (s, 3H), 2.26 (s, 3H). | 527.45 | B | B |
| I- 179 | 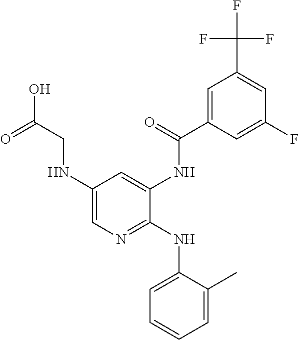 [SMILES: Cc1ccccc1Nc1ncc(NCC(=O)O)cc1NC(=O)c1cc(F)cc(C(F)(F)F)c1] | (400 MHz, dmso) 10.40 (s, 1H), 8.12 (s, 1H), 8.07 (d, J = 9.1 Hz, 1H), 7.97 (d, J = 8.5 Hz, 1H), 7.59 (d, J = 2.7 Hz, 1H), 7.28 (d, J = 8.1 Hz, 1H), 7.12 (d, J = 2.7 Hz, 1H), 7.06 (d, J = 7.4 Hz, 1H), 7.03-6.93 (m, 2H), 6.72 (td, J = 7.4, 1.2 Hz, 1H), 3.62 (s, 3H), 2.13 (s, 3H). | 463.4 | D | |
| I- 180 | 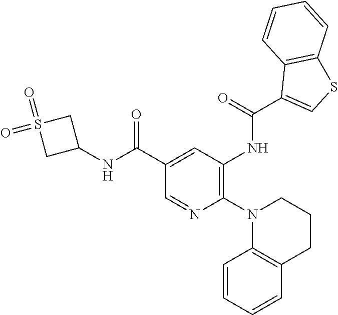 [SMILES: O=C(NC1CS(=O)(=O)C1)c1cnc(N2CCCc3ccccc32)c(NC(=O)c2csc3ccccc23)c1] | | 533.4 | B | D |
| I- 181 | 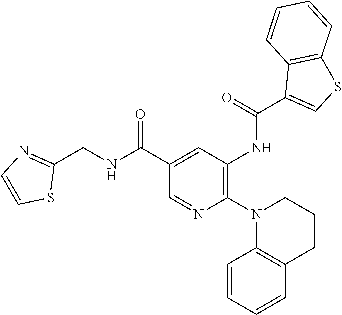 [SMILES: O=C(NCc1nccs1)c1cnc(N2CCCc3ccccc32)c(NC(=O)c2csc3ccccc23)c1] | | 526.3 | B | D |
| I- 182 | 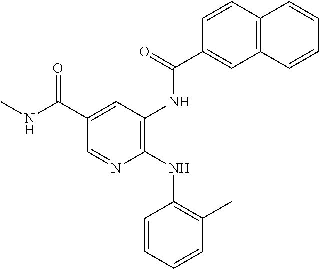 [SMILES: CNC(=O)c1cnc(Nc2ccccc2C)c(NC(=O)c2ccc3ccccc3c2)c1] | | | E | |
| I- 183 | 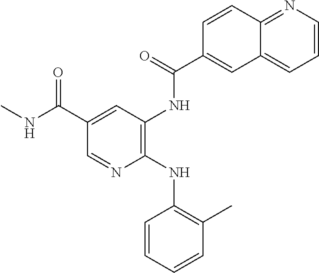 [SMILES: CNC(=O)c1cnc(Nc2ccccc2C)c(NC(=O)c2ccc3ncccc3c2)c1] | | | E | |
| I- 184 | 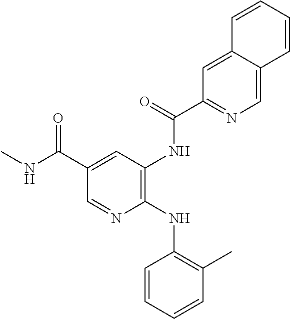 [SMILES: CNC(=O)c1cnc(Nc2ccccc2C)c(NC(=O)c2cc3ccccc3cn2)c1] | | | E | |
| I- 185 | 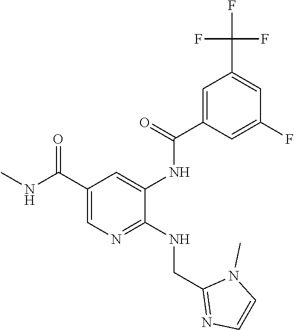 [SMILES: CNC(=O)c1cnc(NCc2nccn2C)c(NC(=O)c2cc(F)cc(C(F)(F)F)c2)c1] | | | E | |
| I- 186 | 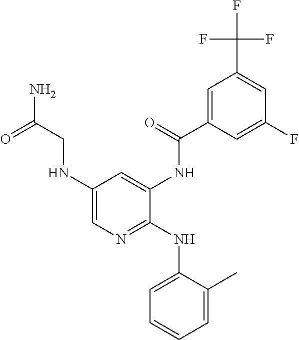 [SMILES: Cc1ccccc1Nc1ncc(NCC(N)=O)cc1NC(=O)c1cc(F)cc(C(F)(F)F)c1] | (400 MHz, dmso) 8.44 (s, 1H), 8.11- 8.00 (m, 1H), 7.89 (d, J = 2.5 Hz, 1H), 7.74 (d, J = 8.7 Hz, 1H), 7.53 (m, 2.5H), 7.51-7.45 (m, 3H), 7.42-7.34 (m, 2.5H), 7.17-7.10 (m, 3H), 7.04 (d, J = 7.1 Hz, 0.5H), 6.97 (t, J = 7.0 Hz, 0.5H), 6.70 (t, J = 6.8 Hz, 0.5H), 6.15 (t, J = 6.1 Hz, 1H), 3.67 (d, J = 5.9 Hz, 2H), 3.57 (d, J = 6.0 Hz, 1H), 2.10 (s, 1.5H), 1.86 (s, 3H). | 462.44 | D | |
| I- 187 | 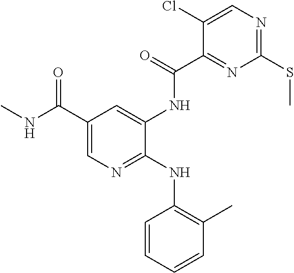 | (400 MHz, cdcl3) 9.42 (s, 1H), 8.69 (s, 1H), 8.56 (d, J = 2.2 Hz, 1H), 8.34 (d, J = 2.2 Hz, 1H), 7.34 (dd, J = 7.8, 1.2 Hz, 1H), 7.22 (d, J = 7.8 Hz, 1H), 7.14 (td, J = 7.8, 1.2 Hz, 1H), 7.00 (td, J = 7.8, 1.2 Hz, 1H), 6.77 (s, 1H), 6.09 (q, J = 4.8 Hz, 1H), 3.01 (d, J = 4.8 Hz, 3H), 2.54 (s, 3H), 2.32 (s, 3H). | 443.37 | E | |
| I- 188 | 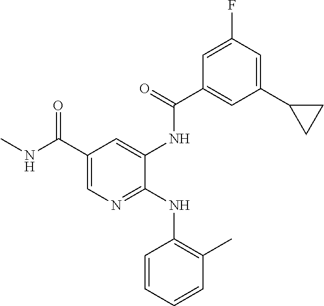 [SMILES: CNC(=O)c1cnc(Nc2ccccc2C)c(NC(=O)c2cc(F)cc(C3CC3)c2)c1] | (400 MHz, cdcl3) 8.48 (s, 1H), 8.30 (d, J = 1.7 Hz, 1H), 8.14 (s, 1H), 7.68-7.42 (m, 1H), 7.32 (d, J = 7.5 Hz, 1H), 7.24- 7.11 (m, 4H), 7.06 (td, J = 7.5, 1.0 Hz, 1H), 6.94-6.87 (m, 1H), 6.25 (q, J = 4.8 Hz, 1H), 2.97 (d, J = 4.8 Hz, 3H), 2.30 (s, 3H), 1.93-1.87 (m, 1H), 1.08-0.99 (m, 2H), 0.74-0.70 (m, 2H). | 419.46 | D | |
| I- 189 | 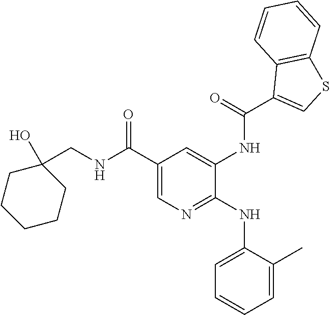 [SMILES: Cc1ccccc1Nc1ncc(C(=O)NCC2(O)CCCCC2)cc1NC(=O)c1csc2ccccc12] | | | C | |
| I- 190 | 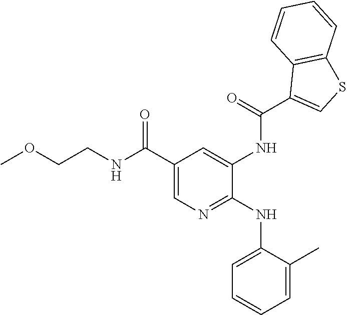 [SMILES: COCCNC(=O)c1cnc(Nc2ccccc2C)c(NC(=O)c2csc3ccccc23)c1] | | | B | D |
| I- 191 | 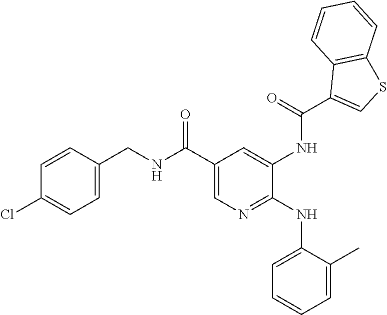 [SMILES: Cc1ccccc1Nc1ncc(C(=O)NCc2ccc(Cl)cc2)cc1NC(=O)c1csc2ccccc12] | | | B | B |
| I- 192 | 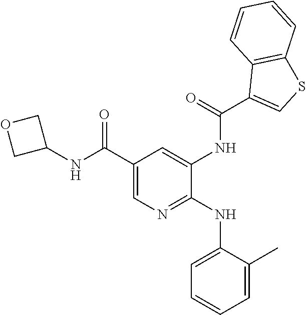 [SMILES: Cc1ccccc1Nc1ncc(C(=O)NC2COC2)cc1NC(=O)c1csc2ccccc12] | | | B | E |
| I- 193 | 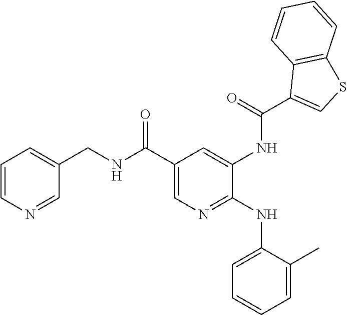 [SMILES: Cc1ccccc1Nc1ncc(C(=O)NCc2cccnc2)cc1NC(=O)c1csc2ccccc12] | | | B | C |
| I- 194 | 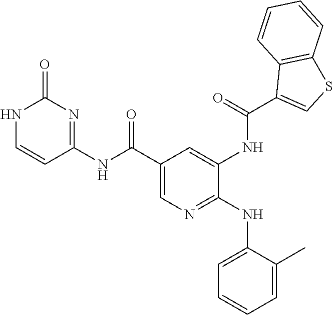 [SMILES: Cc1ccccc1Nc1ncc(C(=O)Nc2cc[nH]c(=O)n2)cc1NC(=O)c1csc2ccccc12] | | | C | |
| I- 195 | 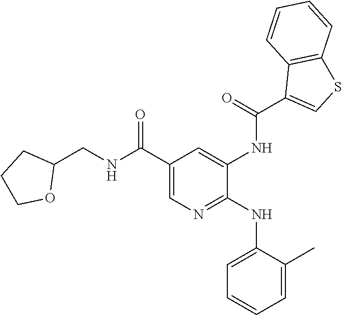 [SMILES: Cc1ccccc1Nc1ncc(C(=O)NCC2CCCO2)cc1NC(=O)c1csc2ccccc12] | | | B | D |
| I- 196 | 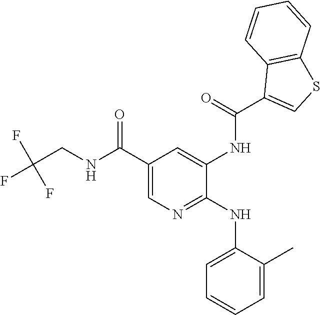 [SMILES: Cc1ccccc1Nc1ncc(C(=O)NCC(F)(F)F)cc1NC(=O)c1csc2ccccc12] | | | B | C |
| I- 197 | 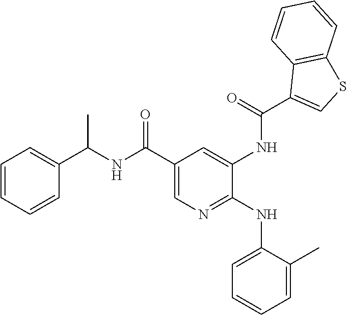 [SMILES: Cc1ccccc1Nc1ncc(C(=O)NC(C)c2ccccc2)cc1NC(=O)c1csc2ccccc12] | | | A | D |
| I- 198 | 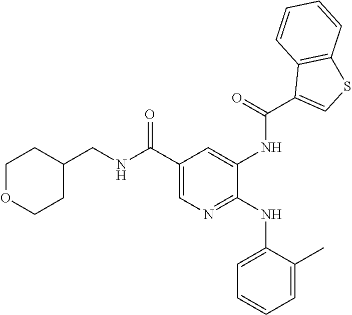 [SMILES: Cc1ccccc1Nc1ncc(C(=O)NCC2CCOCC2)cc1NC(=O)c1csc2ccccc12] | | | B | D |
| I- 199 | 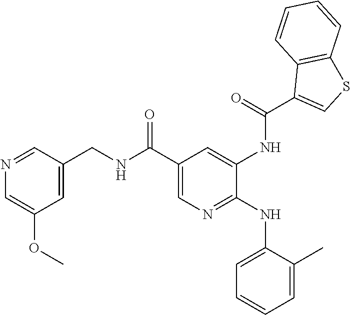 [SMILES: COc1cncc(CNC(=O)c2cnc(Nc3ccccc3C)c(NC(=O)c3csc4ccccc34)c2)c1] | | | B | D |
| I- 200 | 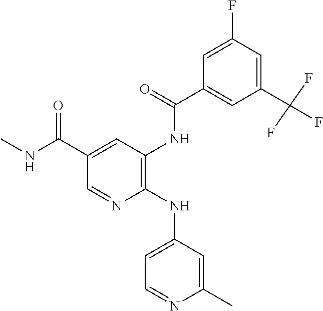 [SMILES: CNC(=O)c1cnc(Nc2ccnc(C)c2)c(NC(=O)c2cc(F)cc(C(F)(F)F)c2)c1] | (400 MHz, dmso) 10.36 (br s, 1H), 8.99 (br s, 1H), 8.74-8.54 (m, 1H), 8.45-8.42 (m, 1H), 8.27 (s, 1H), 8.23 (d, J = 5.6 Hz, 1H), 8.19 (d, J = 9.9 Hz, 1H), 7.99 (br d, J = 6.8 Hz, 1H), 7.59 (d, J = 5.6 Hz, 1H), 7.55 (d, J = 1.2 Hz, 1H), 2.79 (d, J = 4.4 Hz, 3H), 2.39 (s, 3H). | 448.35 | E | |
| I- 201 | 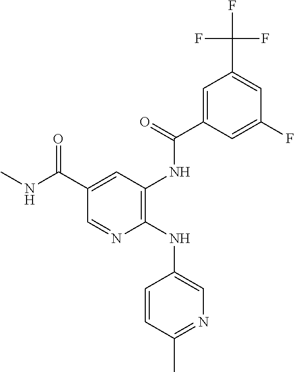 [SMILES: CNC(=O)c1cnc(Nc2ccc(C)nc2)c(NC(=O)c2cc(F)cc(C(F)(F)F)c2)c1] | (400 MHz, dmso) 10.27 (s, 1H), 8.73 (s, 1H), 8.67 (d, J = 2.6 Hz, 1H), 8.55 (d, J = 2.2 Hz, 1H), 8.37 (q, J = 4.5 Hz, 1H), 8.27 (s, 1H), 8.20 (d, J = 9.3 Hz, 1H), 8.06 (d, J = 2.1 Hz, 1H), 8.02 (d, J = 8.4 Hz, 1H), 7.96 (dd, J = 8.4, 2.7 Hz, 1H), 7.20 (d, J = 8.4 Hz, 1H), 2.77 (d, J = 4.5 Hz, 3H), 2.42 (s, 3H). Contains trace impurity and trace of grease. | 448.4 | E | |
| I- 202 | 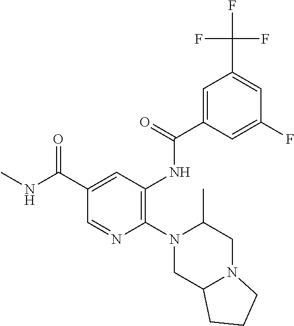 [SMILES: CNC(=O)c1cnc(N2CC3CCCN3CC2C)c(NC(=O)c2cc(F)cc(C(F)(F)F)c2)c1] | (400 MHz, dmso) 10.27 (s, 1H), 8.57 (d, J = 2.1 Hz, 1H), 8.38-8.37 (m 1H), 8.21-8.19 (m, 1H), 8.10 (d, J = 9.5 Hz, 1H), 8.05 (d, J = 2.0 Hz, 1H), 8.02-8.00 (m, 1H), 4.21-4.19 (m, 1H), 3.86 (d, J = 11.0 Hz, 1H), 3.04-2.87 (m, 2H), 2.80 (d, J = 11.8 Hz, 1H), 2.77 (d, J = 4.5 Hz, 3H), 2.19 (d, J = 10.7 Hz, 1H), 1.96-1.92 (m, 2H), 1.70-1.66 (m, 2H), 1.34-1.31 (m, 1H), 1.15 (d, J = 6.5 Hz, 3H). | 480.42 | E | |
| I- 203 | 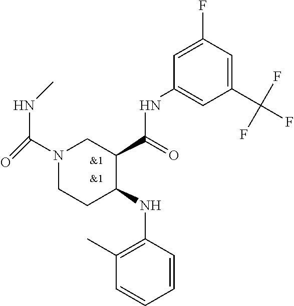 [SMILES: CNC(=O)N1CC[C@H](Nc2ccccc2C)[C@H](C(=O)Nc2cc(F)cc(C(F)(F)F)c2)C1] | (400 MHz, dmso) 10.61 (s, 1H), 7.71- 7.61 (m, 2H), 7.31 (d, J = 8.5 Hz, 1H), 6.97 (dd, J = 14.0, 7.5 Hz, 2H), 6.64 (d, J = 7.9 Hz, 1H), 6.51 (dd, J = 9.5, 5.1 Hz, 2H), 4.44 (d, J = 7.8 Hz, 1H), 4.01 (s, 1H), 3.81 (dd, J = 13.9, 8.0 Hz, 1H), 3.60 (dd, J = 14.0, 3.9 Hz, 1H), 2.91 (dt, J = 8.2, 4.1 Hz, 1H), 2.53 (d, J = 4.3 Hz, 3H), 2.04 (s, 3H), 1.99 (dd, J = 14.2, 7.7 Hz, 1H), 1.61 (d, J = 3.8 Hz, 1H). A signal (1H) is hidden in residual water. | 453.4 | E | |
| I- 204 | 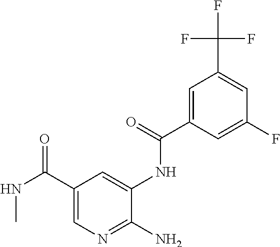 [SMILES: CNC(=O)c1cnc(N)c(NC(=O)c2cc(F)cc(C(F)(F)F)c2)c1] | (400 MHz, dmso) 9.97 (br s, 1H), 8.39 (br s, 1H), 8.20-8.17 (m, 2H), 8.13 (d, J = 8.6 Hz, 1H), 7.94 (d, J = 8.0 Hz, 1H), 7.91 (br s, 1H), 6.53 (s, 2H), 2.73 (d, J = 4.1 Hz, 3H). | 355.31 | E | |
| I- 205 | 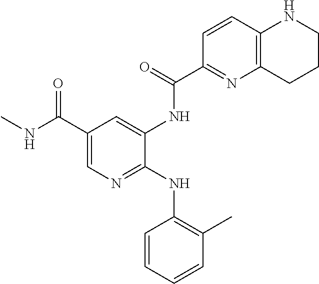 [SMILES: CNC(=O)c1cnc(Nc2ccccc2C)c(NC(=O)c2ccc3c(n2)CCCN3)c1] | (400 MHz, cdcl3) 9.73 (s, 1H), 8.50 (d, J = 2.2 Hz, 1H), 8.24 (d, J = 2.2 Hz, 1H), 7.85 (d, J = 8.4 Hz, 1H), 7.70 (s, 1H), 7.50 (d, J = 7.5 Hz, 1H), 7.21 (d, J = 7.5 Hz, 1H), 7.17 (t, J = 7.5 Hz, 1H), 7.01 (td, J = 7.5, 1.1 Hz, 1H), 6.78 (d, J = 8.4 Hz, 1H), 6.18 (q, J = 4.8 Hz, 1H), 4.39 (s, 1H), 3.40 (t, J = 6.4 Hz, 2H), 3.00 (d, J = 4.8 Hz, 3H), 2.89 (t, J = 6.4 Hz, 2H), 2.32 (s, 3H), 2.05 (p, J = 6.4 Hz, 2H). | 417.46 | E | |
| I- 206 | 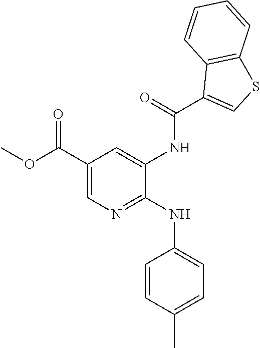 [SMILES: COC(=O)c1cnc(Nc2ccc(C)cc2)c(NC(=O)c2csc3ccccc23)c1] | (400 MHz, cd3od) 8.63 (d, J = 2.1 Hz, 1H), 8.51-8.47 (m, 2H), 8.23 (d, J = 2.1 Hz, 1H), 7.96 (dd, J = 7.0, 1.3 Hz, 1H), 7.50-7.39 (m, 4H), 7.13 (d, J = 8.1 Hz, 2H), 3.89 (s, 3H), 2.30 (s, 3H). Some EtOAc in NMR. | 418.37 | E | |
| I- 207 | 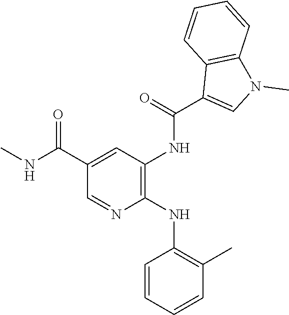 | (400 MHz, cd3od) 8.42 (d, J = 2.3 Hz, 1H), 8.24-8.18 (m, 1H), 8.07 (d, J = 2.3 Hz, 1H), 8.04 (s, 1H), 7.53-7.47 (m, 2H), 7.32-7.27 (m, 1H), 7.23 (t, J = 7.5 Hz, 2H), 7.18 (td, J = 7.5, 1.5 Hz, 1H), 7.06 (td, J = 7.5, 1.5 Hz, 1H), 3.92 (s, 3H), 2.90 (s, 3H), 2.24 (s, 3H). | 414.44 | D | |
| I- 208 | 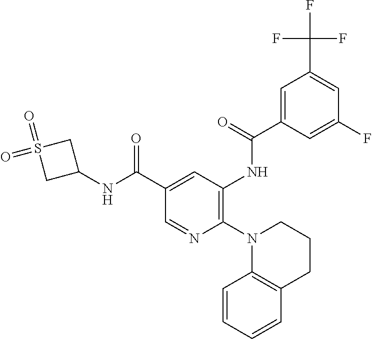 [SMILES: O=C(Nc1cc(C(=O)NC2CS(=O)(=O)C2)cnc1N1CCCc2ccccc21)c1cc(F)cc(C(F)(F)F)c1] | | 563.1 | C | C |
| I- 209 | 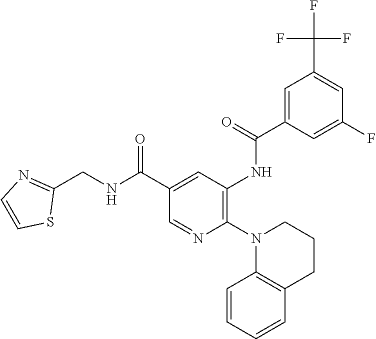 [SMILES: O=C(NCc1nccs1)c1cnc(N2CCCc3ccccc32)c(NC(=O)c2cc(F)cc(C(F)(F)F)c2)c1] | | 556.2 | C | D |
| I- 210 | 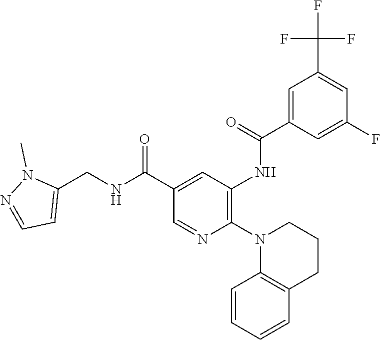 [SMILES: Cn1nccc1CNC(=O)c1cnc(N2CCCc3ccccc32)c(NC(=O)c2cc(F)cc(C(F)(F)F)c2)c1] | | 553.1 | D | E |
| I- 211 | 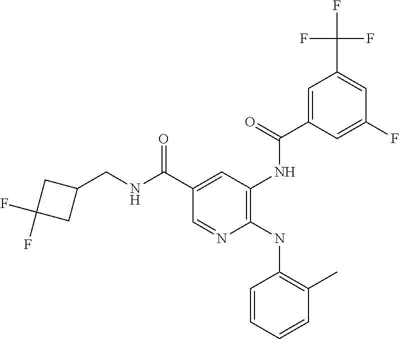 [SMILES: Cc1ccccc1Nc1ncc(C(=O)NCC2CC(F)(F)C2)cc1NC(=O)c1cc(F)cc(C(F)(F)F)c1] | | 537.3 | B | B |
| I- 212 | 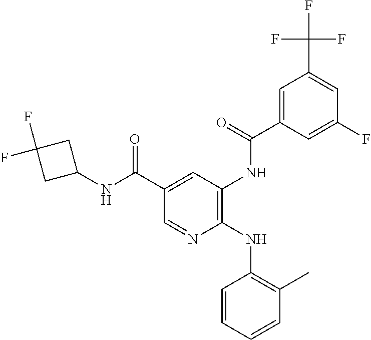 [SMILES: Cc1ccccc1Nc1ncc(C(=O)NC2CC(F)(F)C2)cc1NC(=O)c1cc(F)cc(C(F)(F)F)c1] | | 523.2 | B | B |
| I- 213 | 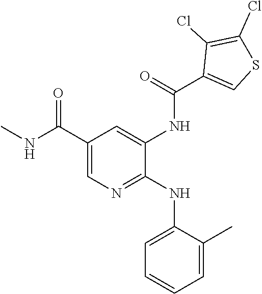 [SMILES: CNC(=O)c1cnc(Nc2ccccc2C)c(NC(=O)c2csc(Cl)c2Cl)c1] | | 435.1 | E | |
| I- 214 | 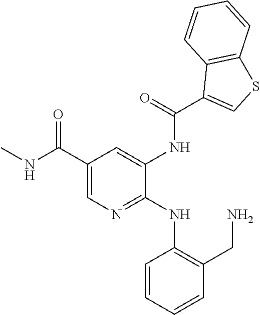 [SMILES: CNC(=O)c1cnc(Nc2ccccc2CN)c(NC(=O)c2csc3ccccc23)c1] | | 432.2 | E | |
| I- 215 | 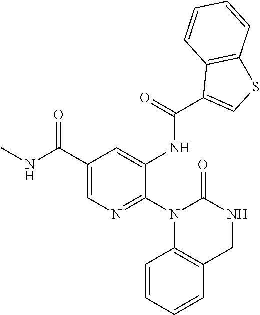 [SMILES: CNC(=O)c1cnc(N2C(=O)NCc3ccccc32)c(NC(=O)c2csc3ccccc23)c1] | | 458 | D | E |
| I- 216 | 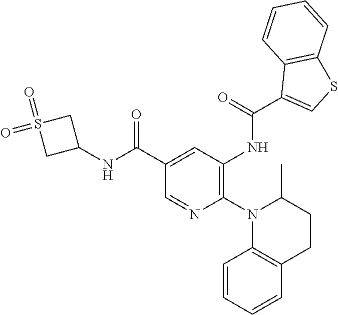 [SMILES: CC1CCc2ccccc2N1c1ncc(C(=O)NC2CS(=O)(=O)C2)cc1NC(=O)c1csc2ccccc12] | (DMSO-d6) 9.50 (s, 1H), 9.20 (d, J = 3.8 Hz, 1H), 8.77 (d, J = 2.3 Hz, 1H), 8.39 (d, J = 2.3 Hz, 1H), 8.16-8.27 (m, 1H), 7.92-8.05 (m, 1H), 7.36-7.45 (m, 2H), 7.29-7.33 (m, 1H), 7.01 (d, J = 6.8 Hz, 1H), 6.80-6.87 (m, 1H), 6.69 (td, J = 7.4, 1.1 Hz, 1H), 6.36 (d, J = 7.3 Hz, 1H), 4.54-4.65 (m, 3H), 4.44-4.53 (m, 1H), 4.24-4.38 (m, 2H), 2.68-2.86 (m, 2H), 2.12-2.25 (m, 1H), 1.74-1.83 (m, 1H), 1.21 (d, J = 6.6 Hz, 3H) | 547.1 | D | |
| I- 217 | 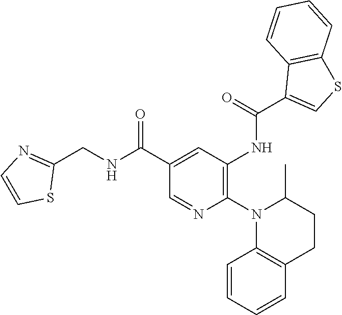 [SMILES: CC1CCc2ccccc2N1c1ncc(C(=O)NCc2nccs2)cc1NC(=O)c1csc2ccccc12] | (DMSO-d6) 9.53 (t, J = 6.1 Hz, 1H), 9.49 (s, 1H), 8.80 (d, J = 2.3 Hz, 1H), 8.43 (d, J = 2.0 Hz, 1H), 8.16-8.26 (m, 1H), 7.96-8.02 (m, 1H), 7.75 (d, J = 3.0 Hz, 1H), 7.65 (d, J = 3.3 Hz, 1H), 7.35- 7.45 (m, 2H), 7.26-7.32 (m, 1H), 7.01 (d, J = 6.6 Hz, 1H), 6.81-6.87 (m, 1H), 6.70 (td, J = 7.4, 1.1 Hz, 1H), 6.38 (d, J = 7.1 Hz, 1H), 4.79 (d, J = 6.1 Hz, 2H), 4.46-4.55 (m, 1H), 2.69-2.88 (m, 3H), 2.13-2.27 (m, 1H), 1.75-1.84 (m, 1H), 1.21 (d, J = 6.6 Hz, 3H) | 540.2 | D | |
| I- 218 | 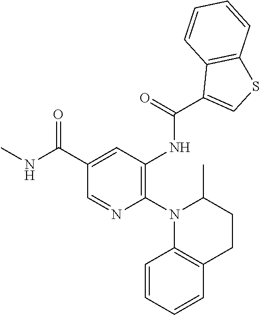 [SMILES: CNC(=O)c1cnc(N2c3ccccc3CCC2C)c(NC(=O)c2csc3ccccc23)c1] | (DMSO-d6) 9.45 (s, 1H), 8.72 (d, J = 2.3 Hz, 1H), 8.57 (q, J = 4.3 Hz, 1H), 8.38 (d, J = 2.3 Hz, 1H), 8.17-8.24 (m, 1H), 7.95-8.03 (m, 1H), 7.37-7.45 (m, 2H), 7.31 (s, 1H), 7.01 (d, J = 7.3 Hz, 1H), 6.79-6.87 (m, 1H), 6.68 (td, J = 7.3, 1.0 Hz, 1H), 6.33 (dd, J = 8.1, 0.8 Hz, 1H), 4.41-4.50 (m, 1H), 2.82 (d, J = 4.5 Hz, 2H), 2.64 (br s, 1H), 2.13-2.24 (m, 1H), 1.73-1.84 (m, 1H), 1.20 (d, J = 6.3 Hz, 3H) | 457.1 | E | |
| I- 219 | 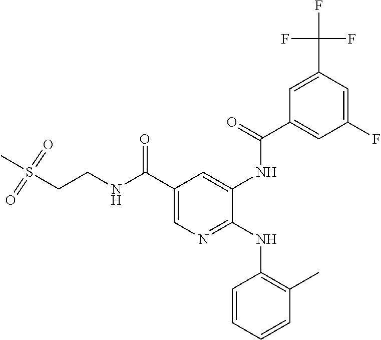 [SMILES: Cc1ccccc1Nc1ncc(C(=O)NCCS(C)(=O)=O)cc1NC(=O)c1cc(F)cc(C(F)(F)F)c1] | | 539.1 | B | B |
| I- 220 | 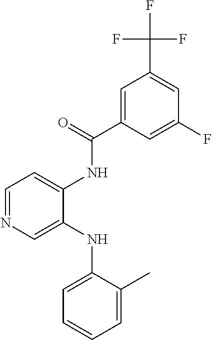 [SMILES: Cc1ccccc1Nc1cnccc1NC(=O)c1cc(F)cc(C(F)(F)F)c1] | (400 MHz, dmso) 10.20 (s, 1H), 8.18 (s, 1H), 8.16 (d, J = 5.2 Hz, 1H), 7.95 (d, J = 8.5 Hz, 1H), 7.91 (s, 1H), 7.88 (d,J = 8.5 Hz, 1H), 7.63 (d, J = 5.2 Hz, 1H), 7.17 (d, J = 7.2 Hz, 1H), 7.14 (s, 1H), 7.04 (td, J = 7.2 Hz, 1H), 6.85 (td, J = 7.4, 6.4 Hz, 1H), 6.77 (d, J = 7.6 Hz, 1H), 2.23 (s, 3H). | 390.32 | D | |
| I- 221 | 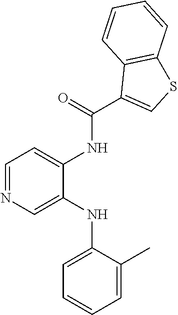 [SMILES: Cc1ccccc1Nc1cnccc1NC(=O)c1csc2ccccc12] | (400 MHz, dmso) 9.96 (s, 1H), 8.38 (s, 1H), 8.36-8.31 (m, 1H), 8.22 (s, 1H), 8.20 (d, J = 5.3 Hz, 1H), 8.10-8.05 (m, 1H), 7.82 (d, J = 5.3 Hz, 1H), 7.51-7.41 (m, 2H), 7.18 (d, J = 7.2 Hz, 1H), 7.07 (s, 1H), 7.05 (td, J = 7.6 Hz, 1H), 6.84 (td, J = 7.6 Hz, 1H), 6.78 (d, J = 7.9 Hz, 1H), 2.24 (s, 3H). | 360.33 | D | |
| I- 222 | 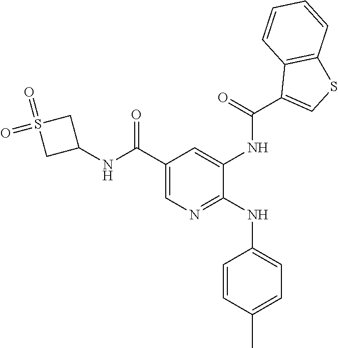 [SMILES: Cc1ccc(Nc2ncc(C(=O)NC3CS(=O)(=O)C3)cc2NC(=O)c2csc3ccccc23)cc1] | (400 MHz, dmso) 9.94 (s, 1H), 8.95 (d, J = 2.1 Hz, 1H), 8.71-8.70 (m, 2H), 8.55 (d, J = 2.1 Hz, 1H), 8.49 (d, J = 8.5 Hz, 1H), 8.16 (s, 1H), 8.08 (dd, J = 8.5, 1.2 Hz, 1H), 7.53 (d, J = 8.5 Hz, 2H), 7.51-7.40 (m, 2H), 7.10 (d, J = 8.5 Hz, 2H), 4.60-4.50 (m, 3H), 4.33-4.22 (m, 2H), 2.25 (s, 3H). | 507.38 | D | |
| I- 223 | 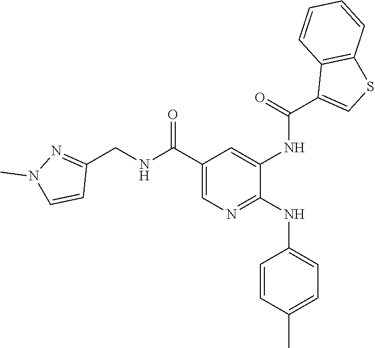 [SMILES: Cc1ccc(Nc2ncc(C(=O)NCc3ccn(C)n3)cc2NC(=O)c2csc3ccccc23)cc1] | (400 MHz, cdcl3) 8.60 (s, 1H, NH), 8.53-8.47 (m, 2H), 8.18 (s, 1H), 8.04 (s, 1H), 8.01 (s, 1H, NH), 7.87 (dd, J = 7.1, 0.9 Hz, 1H), 7.52 (s, 1H, NH), 7.48-7.37 (m, 2H), 7.26 (d, J = 8.2 Hz, 2H), 7.17 (d, J = 2.2 Hz, 1H), 7.04 (d, J = 8.2 Hz, 2H), 6.12 (d, J = 2.2 Hz, 1H), 4.57 (d, J = 5.4 Hz, 2H), 3.77 (s, 3H), 2.24 (s, 3H). | 497.44 | D | |
| I- 224 | 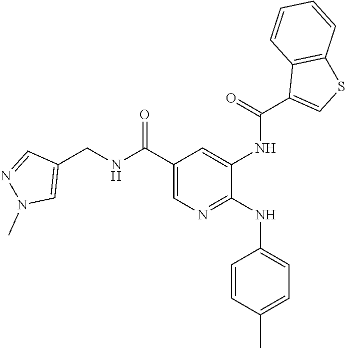 [SMILES: Cc1ccc(Nc2ncc(C(=O)NCc3cnn(C)c3)cc2NC(=O)c2csc3ccccc23)cc1] | (400 MHz, cdcl3) 8.46-8.44 (m, 2H), 8.20 (s, 1H), 8.08 (d, J = 2.1 Hz, 1H), 8.02 (s, 1H), 7.91 (s, 1H), 7.88 (dd, J = 7.1, 1.2 Hz, 1H), 7.49-7.40 (m, 3H), 7.31 (s, 1H), 7.24 (d, J = 8.4 Hz, 2H), 7.09 (d, J = 8.4 Hz, 2H), 6.38 (t, J = 5.4 Hz, 1H), 4.39 (d, J = 5.4 Hz, 2H), 3.83 (s, 3H), 2.28 (s, 3H). | 497.43 | E | |
| I- 225 | 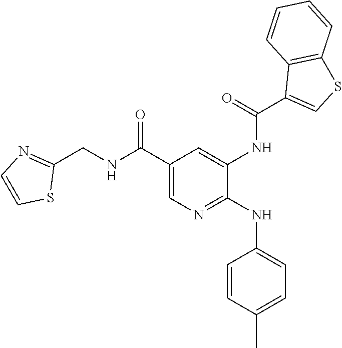 [SMILES: Cc1ccc(Nc2ncc(C(=O)NCc3nccs3)cc2NC(=O)c2csc3ccccc23)cc1] | (400 MHz, cdcl3) 8.52 (s, 1H), 8.46 (d, J = 7.4 Hz, 1H), 8.25-8.08 (m, 2H), 7.84 (d, J = 7.4 Hz, 1H), 7.59 (s, 1H), 7.48-7.36 (m, 2H), 7.25 (d, J = 8.2 Hz, 2H), 7.19 (d, J = 3.2 Hz, 1H), 7.03 (d, J = 8.2 Hz, 2H), 4.86 (d, J = 5.1 Hz, 2H), 2.21 (s, 3H). | 500.37 | D | |
| I- 226 | 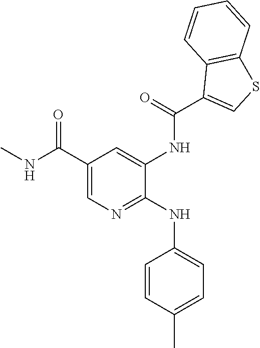 [SMILES: CNC(=O)c1cnc(Nc2ccc(C)cc2)c(NC(=O)c2csc3ccccc23)c1] | (400 MHz, cd3od) 8.51-8.46 (m, 2H), 8.44 (s, 1H), 8.13 (d, J = 2.3 Hz, 1H), 7.98-7.93 (m, 1H), 7.49-7.37 (m, 4H), 7.11 (d, J = 8.1 Hz, 2H), 2.91 (s, 3H), 2.29 (s, 3H). | 417.38 | E | |
| I- 227 | 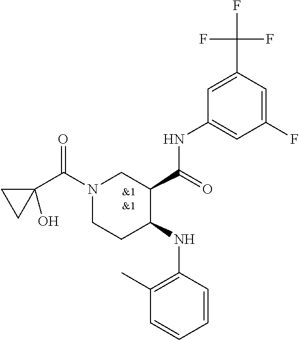 [SMILES: Cc1ccccc1N[C@H]1CCN(C(=O)C2(O)CC2)C[C@H]1C(=O)Nc1cc(F)cc(C(F)(F)F)c1] | (400 MHz, dmso) 10.62 (s, 1H), 7.65 (s, 1H), 7.62 (s, 1H),, 7.30 (d, J = 8.4 Hz, 1H), 6.99 (dd, J = 10.9, 4.5 Hz, 1H), 6.95 (d, J = 7.3 Hz, 1H), 6.69 (d, J = 7.9 Hz, 1H), 6.56-6.48 (m, 1H), 6.36 (s, 1H), 4.36 (d, J = 7.9 Hz, 1H), 4.12- 4.03 (m, 1H), 3.02 (m, 1H), 2.15 (m, 1H), 2.03 (s, 3H), 1.72 (m, 1H), 0.94 (m, 1H), 0.70 (m, 2H | 480.42 | E | |
| I- 228 | 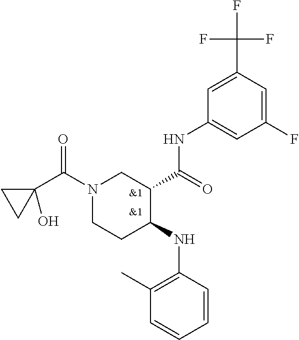 [SMILES: Cc1ccccc1N[C@H]1CCN(C(=O)C2(O)CC2)C[C@@H]1C(=O)Nc1cc(F)cc(C(F)(F)F)c1] | (400 MHz, dmso) 10.53 (s, 1H), 7.67 (s, 1H), 7.62 (dt, J = 11.1, 2.0 Hz, 1H), 7.29 (d, J = 8.6 Hz, 1H), 6.93 (td, J = 7.8, 1.0 Hz, 1H), 6.86 (dd, J = 7.3, 0.7 Hz, 1H), 6.69-6.65 (m, 1H), 6.42 (td, J = 7.3, 0.9 Hz, 1H), 6.38 (s, 1H), 4.56 (bs, 2H), 4.27 (d, J = 9.8 Hz, 1H), 3.93- 3.80 (m, 1H), 2.62 (m, 1H), 2.09 (ddd, J = 10.9, 5.2, 1.4 Hz, 1H), 1.98 (s, 3H), 1.25 (d, J = 23.3 Hz, 1H), 1.01-0.81 (m, 2H), 0.78 (s, 1H). | 480.42 | E | |
| I- 229 | 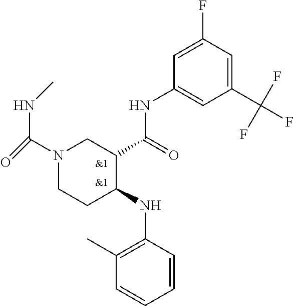 | (400 MHz, dmso) 10.47 (d, J = 7.7 Hz, 1H), 7.68 (s, 1H), 7.62 (dt, J = 11.1, 2.1 Hz, 1H), 7.29 (dt, J = 8.5, 1.6 Hz, 1H), 6.92 (td, J = 8.1, 1.3 Hz, 1H), 6.87-6.83 (m, 1H), 6.68-6.63 (m, 1H), 6.60-6.54 (m, 1H), 6.42 (td, J = 7.4, 0.9 Hz, 1H), 4.24 (d, J = 9.7 Hz, 1H), 4.19-4.11 (m, 1H), 4.00-3.92 (m, 1H), 3.82-3.71 (m, 1H), 2.95-2.80 (m, 2H), 2.60-2.53 (m, 4H (one doublet of 3 H for the NMe)), 1.96 (s, 3H), 1.26-1.12 (m, 1H). | 453.41 | E | |
| I- 230 | 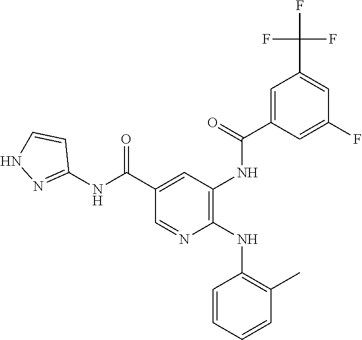 [SMILES: Cc1ccccc1Nc1ncc(C(=O)Nc2cc[nH]n2)cc1NC(=O)c1cc(F)cc(C(F)(F)F)c1] | | | D | |
| I- 231 | 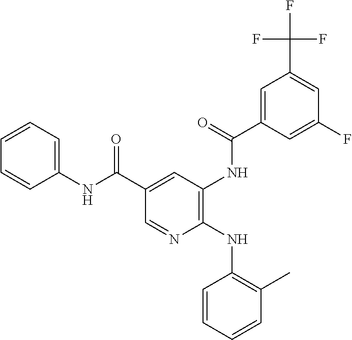 [SMILES: Cc1ccccc1Nc1ncc(C(=O)Nc2ccccc2)cc1NC(=O)c1cc(F)cc(C(F)(F)F)c1] | | | D | |
| I- 232 | 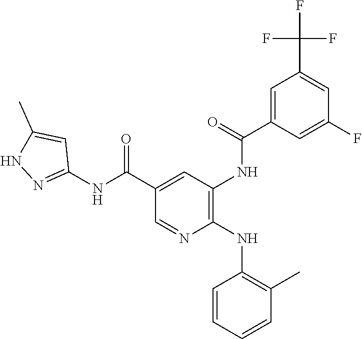 [SMILES: Cc1cc(NC(=O)c2cnc(Nc3ccccc3C)c(NC(=O)c3cc(F)cc(C(F)(F)F)c3)c2)n[nH]1] | | | B | B |
| I- 233 | 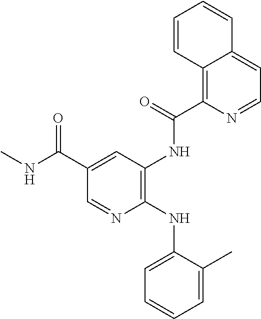 [SMILES: CNC(=O)c1cnc(Nc2ccccc2C)c(NC(=O)c2nccc3ccccc23)c1] | | | E | |
| I- 234 | 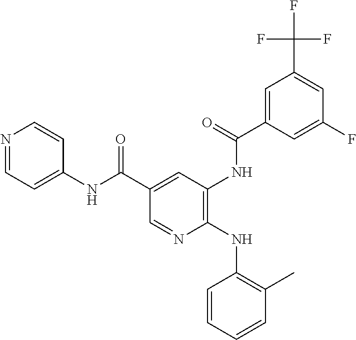 [SMILES: Cc1ccccc1Nc1ncc(C(=O)Nc2ccncc2)cc1NC(=O)c1cc(F)cc(C(F)(F)F)c1] | | | B | A |
| I- 235 | 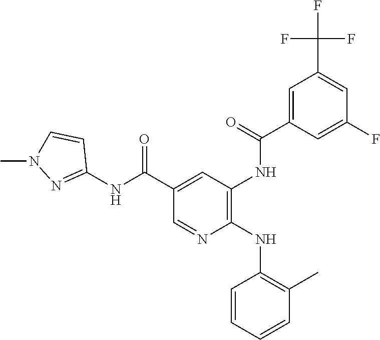 [SMILES: Cc1ccccc1Nc1ncc(C(=O)Nc2ccn(C)n2)cc1NC(=O)c1cc(F)cc(C(F)(F)F)c1] | | | C | |
| I- 236 | 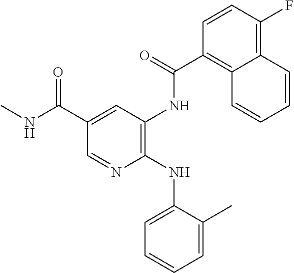 [SMILES: CNC(=O)c1cnc(Nc2ccccc2C)c(NC(=O)c2ccc(F)c3ccccc23)c1] | | | E | |
| I- 237 | 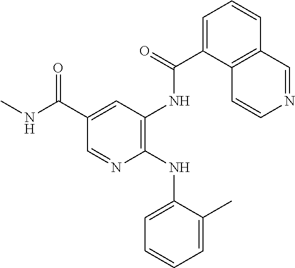 [SMILES: CNC(=O)c1cnc(Nc2ccccc2C)c(NC(=O)c2cccc3cnccc23)c1] | | | E | |
| I- 238 | 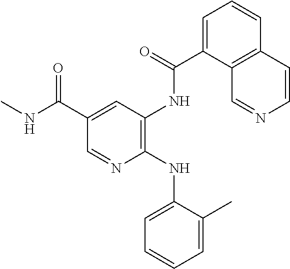 [SMILES: CNC(=O)c1cnc(Nc2ccccc2C)c(NC(=O)c2cccc3ccncc23)c1] | | | E | |
| I- 239 | 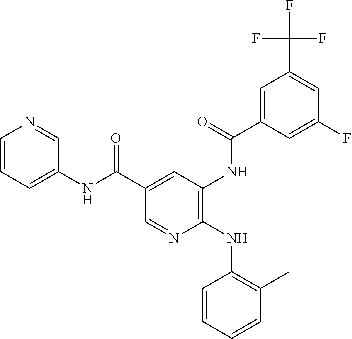 [SMILES: Cc1ccccc1Nc1ncc(C(=O)Nc2cccnc2)cc1NC(=O)c1cc(F)cc(C(F)(F)F)c1] | | | B | B |
| I- 240 | 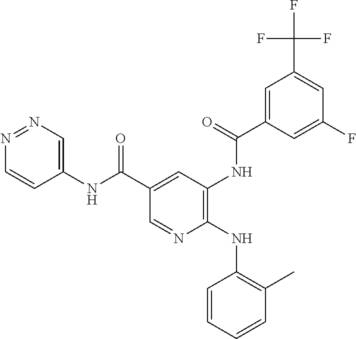 [SMILES: Cc1ccccc1Nc1ncc(C(=O)Nc2ccnnc2)cc1NC(=O)c1cc(F)cc(C(F)(F)F)c1] | | | B | A |
| I- 241 | 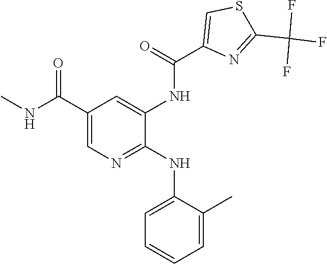 [SMILES: CNC(=O)c1cnc(Nc2ccccc2C)c(NC(=O)c2csc(C(F)(F)F)n2)c1] | (DMSO-d6, 400 MHz) 10.22 (s, 1H), 8.86 (s, 1H), 8.42 (br d, J = 1.5 Hz, 1H), 8.28 (br d, J = 4.4 Hz, 1H), 8.20 (br s, 1H), 8.01 (s, 1H), 7.32 (br d, J = 7.6 Hz, 1H), 7.21 (br d, J = 7.3 Hz, 1H), 7.16 (br t, J = 7.4 Hz, 1H), 7.02- 7.09 (m, 1H), 2.76 (br d, J = 4.4 Hz, 3H), 2.14 ppm (s, 3H). | 436 | E | |
| I- 242 | 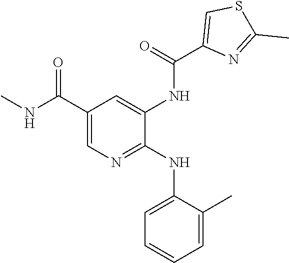 | | 382 | E | |
| I- 243 | 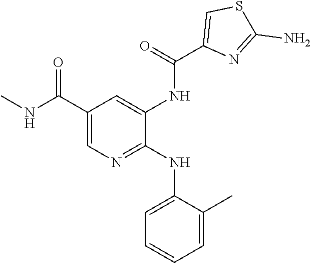 [SMILES: CNC(=O)c1cnc(Nc2ccccc2C)c(NC(=O)c2csc(N)n2)c1] | | 383.1 | E | |
| I- 244 | 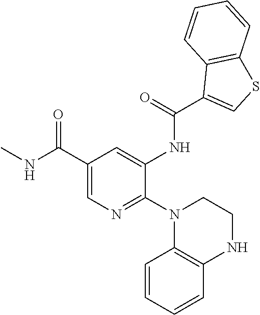 [SMILES: CNC(=O)c1cnc(N2CCNc3ccccc32)c(NC(=O)c2csc3ccccc23)c1] | | 443.9 | E | |
| I- 245 | 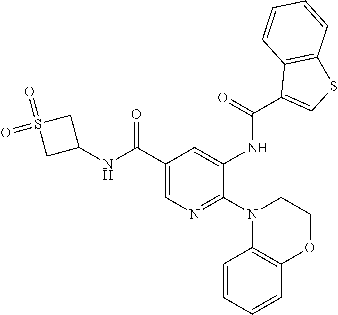 [SMILES: O=C(NC1CS(=O)(=O)C1)c1cnc(N2CCOc3ccccc32)c(NC(=O)c2csc3ccccc23)c1] | | 535 | B | C |
| I- 246 | 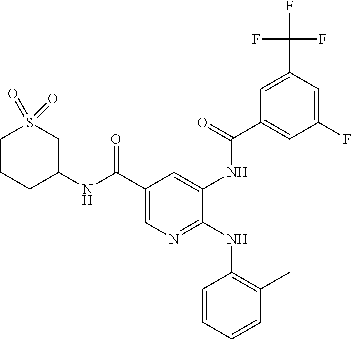 [SMILES: Cc1ccccc1Nc1ncc(C(=O)NC2CCCS(=O)(=O)C2)cc1NC(=O)c1cc(F)cc(C(F)(F)F)c1] | | 565.2 | B | A |
| I- 247 | 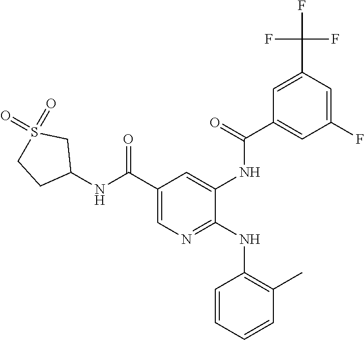 [SMILES: Cc1ccccc1Nc1ncc(C(=O)NC2CCS(=O)(=O)C2)cc1NC(=O)c1cc(F)cc(C(F)(F)F)c1] | | 551.2 | B | A |
| I- 248 | 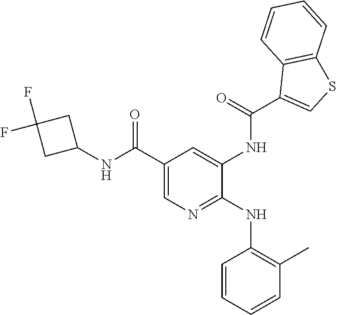 [SMILES: Cc1ccccc1Nc1ncc(C(=O)NC2CC(F)(F)C2)cc1NC(=O)c1csc2ccccc12] | | 493.1 | B | B |
| I- 249 | 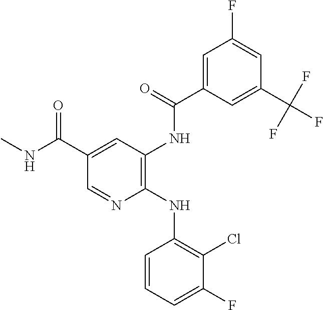 [SMILES: CNC(=O)c1cnc(Nc2cccc(F)c2Cl)c(NC(=O)c2cc(F)cc(C(F)(F)F)c2)c1] | | 485 | B | B |
| I- 250 | 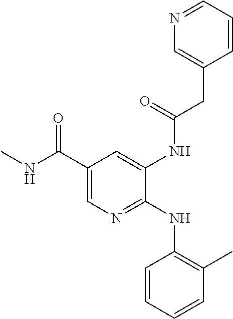 [SMILES: CNC(=O)c1cnc(Nc2ccccc2C)c(NC(=O)Cc2cccnc2)c1] | (400 MHz, dmso) 9.81 (s, 1H), 8.53 (d, J = 2.2 Hz, 1H), 8.44 (dd, J = 4.6, 1.6 Hz, 1H), 8.33 (d, J = 2.2 Hz, 1H), 8.24 (q, J = 4.6 Hz, 1H), 7.95-7.90 (m, 2H), 7.76 (d, J = 7.8 Hz, 1H), 7.41 (d, J = 7.8 Hz, 1H), 7.34 (dd, J = 7.8, 4.6 Hz, 1H), 7.19 (d, J = 7.8 Hz, 1H), 7.15 (t, J = 7.8 Hz, 1H), 7.02 (t, J = 7.8 Hz, 1H), 3.76 (s, 2H), 2.70 (d, J = 4.6 Hz, 3H), 2.07 (s, 3H). | 376.41 | E | |
| I- 251 | 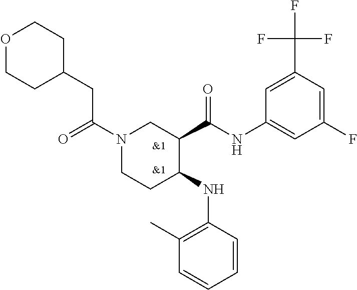 [SMILES: Cc1ccccc1N[C@H]1CCN(C(=O)CC2CCOCC2)C[C@H]1C(=O)Nc1cc(F)cc(C(F)(F)F)c1] | (400 MHz, dmso) 10.63 (s, 0.4H), 10.53 (s, 0.6H), 7.67 (m, 1.4H), 7.57 (dd, J = 10.9, 2.0 Hz, 0.6H), 7.33 (td, J = 10.4, 1.3 Hz, 1H), 7.00 (t, J = 7.8 Hz, 1H), 6.95 (d, J = 7.3 Hz, 1H), 6.70 (m, J = 7.9, 4.6 Hz, 1H), 6.52 (t, J = 7.7 Hz, 1H), 4.37 (dd, J = 16.4, 8.6 Hz, 0.6H), 4.28 (dd, J = 13.6, 5.7 Hz, 0.4H), 4.10- 3.96 (m, 1H), 3.86-3.64 (m, 2H), 3.19 (m, 1.2H), 3.04-2.92 (m, 0.8H), 2.33 (dd, J = 14.9, 7.0 Hz, 0.6H), 2.26-2.08 (m, 1.6H), 2.01 (d, J = 5.9 Hz, 1H), 1.93-1.78 (m, 0.6H), 1.78-1.70 (m, 0.4H), 1.70-1.60 (m, 0.4H), 1.53 (m, 1.4H), 1.40 (m, 0.4H), 1.30-1.03 (m, 1.4H), 1.04-0.90 (m, 0.4H). | 522.5 | E | |
| I- 252 | 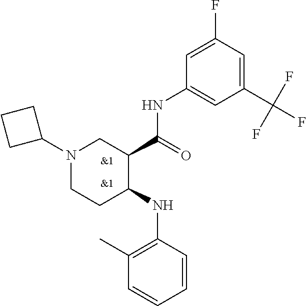 [SMILES: Cc1ccccc1N[C@H]1CCN(C2CCC2)C[C@H]1C(=O)Nc1cc(F)cc(C(F)(F)F)c1] | (400 MHz, dmso) 11.02 (s, 1H), 8.22 (s, 0.4H), 7.75-7.68 (m, 2H (Incl. one singlet)), 7.37-7.32 (dt, J = 8, 1.8 Hz, 1H), 7.02-6.94 (m, 2H), 6.62 (d, J = 8.1 Hz, 1H), 6.54-6.48 (td, J = 7.3, 0.8 Hz, 1H), 4.47 (m, 1H), 3.82 (bs, 1H), 2.99- 2.92 (m, 1H), 2.91-2.74 (m , 2H, Including quintet of 1H, J = 7.6 Hz), 2.48-2.36 (m, 1H), 2.30-2.15 (m, 11H), 2.04 (s, 3H), 2.00 (m, 2H), 1.97-1.89 (m, 1H), 1.89-1.68 (m, 3H), 1.68-1.58 (m, 2H). | 450.42 | E | |
| I- 253 | 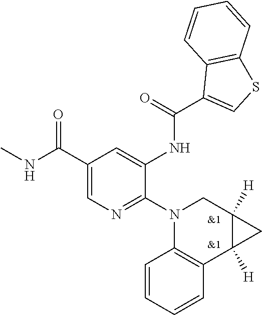 [SMILES: CNC(=O)c1cnc(N2C[C@H]3C[C@H]3c3ccccc32)c(NC(=O)c2csc3ccccc23)c1] | | 455.1 | D | |
| I- 254 | 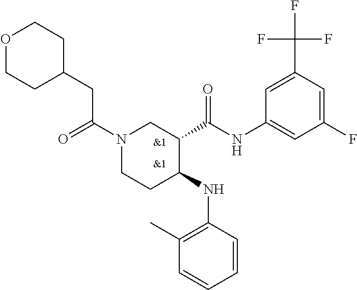 [SMILES: Cc1ccccc1N[C@H]1CCN(C(=O)CC2CCOCC2)C[C@@H]1C(=O)Nc1cc(F)cc(C(F)(F)F)c1] | (400 MHz, dmso) 10.54 (s, 0.6H), 10.52 (s, 0.4H), 7.70 (s, 1H), 7.66 (dd, J = 10.9, 1.9 Hz, 4H), 7.32 (d, J = 8.8 Hz, 1H), 6.95 (dd, J = 13.8, 6.5 Hz, 1H), 6.88 (d, J = 7.4 Hz, 1H), 6.68 (dd, J = 12.9, 8.1 Hz, 1H), 6.45 (m, 1H), 4.61 (d, J = 10.9 Hz, 0.6H), 4.45 (d, J = 13.3 Hz, 0.4H), 4.34 (t, J = 8.8 Hz, 1H), 4.17 (d, J = 13.0 Hz, 0.4H), 3.96 (d, J = 13.3 Hz, 0.6H), 3.92-3.77 (m, 3H), 3.34-3.16 (m, 3H), 2.77 (dd, J = 27.0, 14.5 Hz, 0.6H), 2.67-2.51 (m, 1.4H), 2.43-2.23 (m, 1.6H), 2.09 (t, J = 12.4 Hz, 0.6H), 1.99 (two singlet, 3H), 1.91 (m, 0.8H), 1.58 (m, 1.6H), 1.23 (m, 3H). | 522.5 | E | |
| I- 255 | 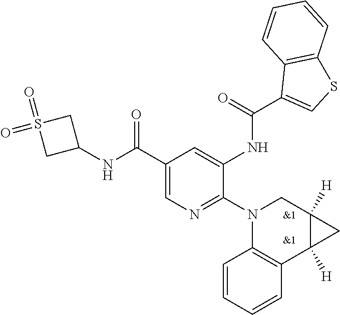 [SMILES: O=C(NC1CS(=O)(=O)C1)c1cnc(N2C[C@H]3C[C@H]3c3ccccc32)c(NC(=O)c2csc3ccccc23)c1] | | 545.1 | C | |
| I- 256 | 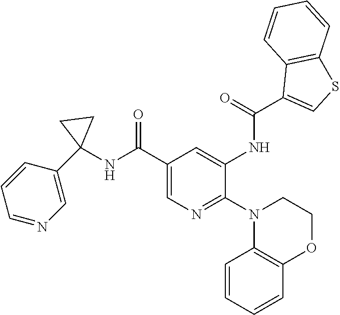 [SMILES: O=C(NC1(c2cccnc2)CC1)c1cnc(N2CCOc3ccccc32)c(NC(=O)c2csc3ccccc23)c1] | | 548 | B | B |
| I- 257 | 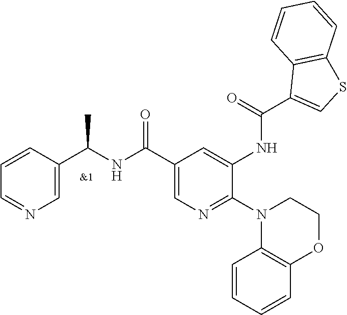 [SMILES: C[C@@H](NC(=O)c1cnc(N2CCOc3ccccc32)c(NC(=O)c2csc3ccccc23)c1)c1cccnc1] | | 536.1 | A | C |
| I- 258 | 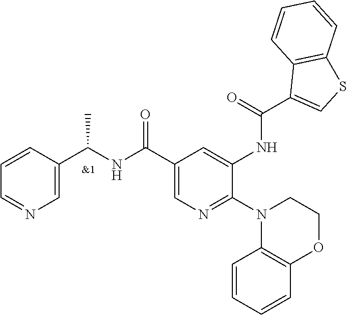 [SMILES: C[C@H](NC(=O)c1cnc(N2CCOc3ccccc32)c(NC(=O)c2csc3ccccc23)c1)c1cccnc1] | | 536.1 | B | D |
| I- 259 | 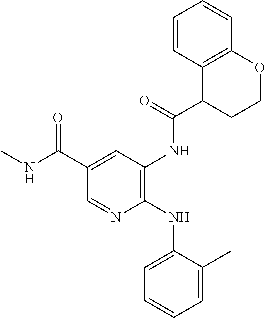 [SMILES: CNC(=O)c1cnc(Nc2ccccc2C)c(NC(=O)C2CCOc3ccccc32)c1] | (400 MHz, DMSO-d6) 9.96 (s, 1H), 8.40 (d, J = 2.27 Hz, 1H), 8.33 (q, J = 4.13 Hz, 1H), 8.03 (d, J = 2.27 Hz, 1H), 7.90 (s, 1H), 7.45-7.63 (m, 1H), 7.28-7.41 (m, 1H), 7.13-7.27 (m, 3H), 7.02-7.10 (m, 1H), 6.88 (dt, J = 1.26, 7.45 Hz, 1H), 6.82 (dd, J = 1.14, 8.21 Hz, 1H), 4.42 (ddd, J = 3.41, 8.02, 11.05 Hz, 2H), 4.11-4.30 (m, 1H), 3.98-4.09 (m, 1H), 3.41-3.65 (m, 2H), 2.76 (d, J = 4.55 Hz, 3H), 2.68 (s, 1H), 2.57 (br d, J = 8.08 Hz, 1H), 2.12-2.29 (m, 5H), 2.08-2.09 (m, 1H), 1.18 (br t, J = 7.07 Hz, 1H) | 417 | E | |
| I- 260 | 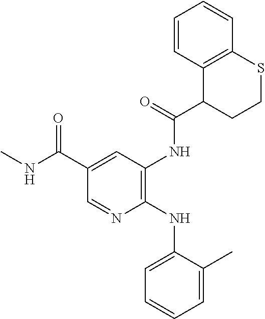 [SMILES: CNC(=O)c1cnc(Nc2ccccc2C)c(NC(=O)C2CCSc3ccccc32)c1] | (400 MHz, DMSO-d6) Shift 9.81-9.96 (m, 1H), 8.38-8.44 (m, 1H), 8.26-8.36 (m, 1H), 8.04 (d, J = 2.27 Hz, 1H), 7.88 (s, 1H), 7.46-7.62 (m, 1H), 7.29-7.35 (m, 1H), 7.22-7.26 (m, 1H), 7.16-7.22 (m, 1H), 7.11-7.15 (m, 2H), 7.02-7.09 (m, 2H), 3.90-4.17 (m, 2H), 2.70-2.89 (m, 3H), 1.73-2.22 (m, 3H) | 433 | E | |
| I- 261 | 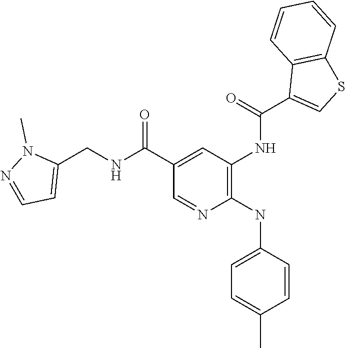 [SMILES: Cc1ccc(Nc2ncc(C(=O)NCc3ccnn3C)cc2NC(=O)c2csc3ccccc23)cc1] | (400 MHz, dmso) 9.89 (s, 1H), 8.84 (t, J = 5.5 Hz, 1H), 8.68 (s, 1H), 8.60 (s, 1H), 8.54 (d, J = 2.2 Hz, 1H), 8.49-8.42 (m, 1H), 8.14 (d, J = 2.2 Hz, 1H), 8.09- 8.03 (m, 1H), 7.53-7.48 (m, 2H), 7.43 (pd, J = 7.1, 1.8 Hz, 2H), 7.27 (d, J = 1.8 Hz, 1H), 7.07 (d, J = 8.3 Hz, 2H), 6.13 (d, J = 1.8 Hz, 1H), 4.48 (d, J = 5.5 Hz, 2H), 3.79 (s, 3H), 2.22 (s, 3H). | 497.42 | D | |
| I- 262 | 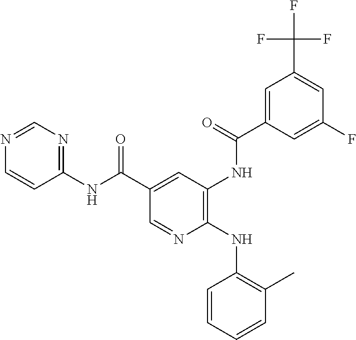 [SMILES: Cc1ccccc1Nc1ncc(C(=O)Nc2ccncn2)cc1NC(=O)c1cc(F)cc(C(F)(F)F)c1] | | | C | |
| I- 263 | 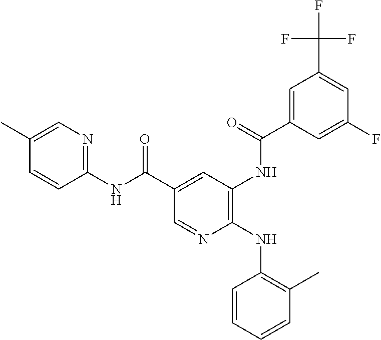 [SMILES: Cc1ccc(NC(=O)c2cnc(Nc3ccccc3C)c(NC(=O)c3cc(F)cc(C(F)(F)F)c3)c2)nc1] | | | D | |
| I- 264 | 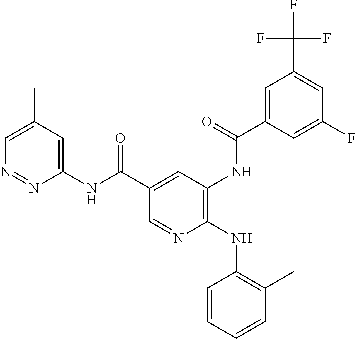 [SMILES: Cc1cnnc(NC(=O)c2cnc(Nc3ccccc3C)c(NC(=O)c3cc(F)cc(C(F)(F)F)c3)c2)c1] | | | D | |
| I- 265 | 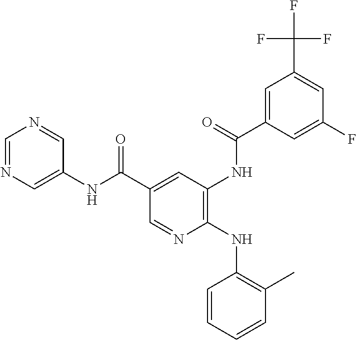 [SMILES: Cc1ccccc1Nc1ncc(C(=O)Nc2cncnc2)cc1NC(=O)c1cc(F)cc(C(F)(F)F)c1] | | | C | |
| I- 266 | 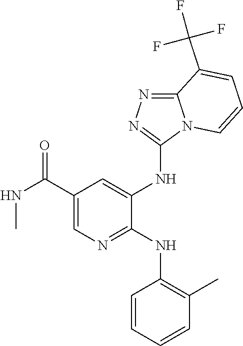 [SMILES: CNC(=O)c1cnc(Nc2ccccc2C)c(Nc2nnc3c(C(F)(F)F)cccn23)c1] | (400 MHz, dmso) 9.05 (dd, J = 7.5, 1.2 Hz, 1H), 8.88 (d, J = 2.0 Hz, 1H), 8.73 (s, 1H), 8.71 (d, J = 2.0 Hz, 1H), 8.43 (s, 2H), 8.03 (dd, J = 7.5, 1.2 Hz, 1H), 7.40-7.35 (m, 2H), 7.31 (td, J = 7.5, 1.2 Hz, 1H), 7.21 (dd, J = 7.5, 1.2 Hz, 1H), 7.14 (td, J = 7.5, 1.2 Hz, 1H), 2.87 (s, 3H), 2.11 (s, 3H). | 418.37 | E | |
| I- 267 | 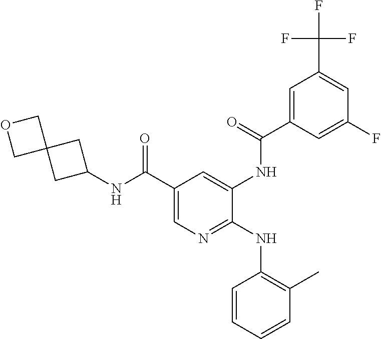 [SMILES: Cc1ccccc1Nc1ncc(C(=O)NC2CC3(COC3)C2)cc1NC(=O)c1cc(F)cc(C(F)(F)F)c1] | (400 MHz, dmso) 8.45-8.32 (m, 3H), 8.23 (s, 1H), 8.17 (d, J = 8.9 Hz, 1H), 8.02 (s, 1H), 7.98 (d, J = 8.5 Hz, 1H), 7.34 (d, J = 5.1 Hz, 1H), 7.23 (d, J = 7.2 Hz, 1H), 7.17 (td, J = 7.5, 1.2 Hz, 1H), 7.07 (td, J = 7.4, 1.1 Hz, 1H), 4.62 (s, 2H), 4.50 (s, 2H), 4.27-4.14 (m, 1H), 2.62-2.53 (m, 2H), 2.26-2.18 (m, 2H), 2.15 (s, 3H). | 529.41 | B | A |
| I- 268 | 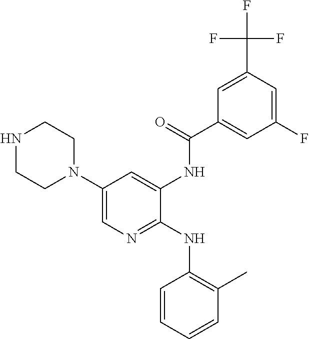 [SMILES: Cc1ccccc1Nc1ncc(N2CCNCC2)cc1NC(=O)c1cc(F)cc(C(F)(F)F)c1] | (400 MHz, cd3od) 8.21 (s, 1H), 8.13 (d, J = 2.6 Hz, 1H), 8.05 (d, J = 8.8 Hz, 1H), 7.77 (d, J = 8.2 Hz, 1H), 7.41 (dd, J = 5.2, 3.7 Hz, 1H), 7.36-7.31 (m, 2H), 7.30 (d, J = 2.6 Hz, 1H), 7.27 (dd, J = 5.5, 3.8 Hz, 1H), 3.38 (s, 8H), 2.28 (s, 3H). | 474.45 | C | |
| I- 269 | 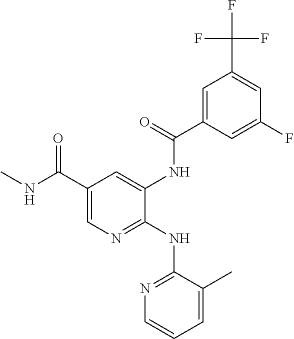 [SMILES: CNC(=O)c1cnc(Nc2ncccc2C)c(NC(=O)c2cc(F)cc(C(F)(F)F)c2)c1] | (400 MHz, dmso) 10.25 (s, 1H), 8.94 (s, 1H), 8.50 (d, J = 1.8 Hz, 1H), 8.40 (q, J = 4.3 Hz, 1H), 8.17 (s, 1H), 8.13 (d, J = 3.6 Hz, 1H), 8.04 (s, 1H), 8.03 (m, 1H), 7.95 (d, J = 8.5 Hz, 1H), 7.58 (d, J = 6.9 Hz, 1H), 7.03 (dd, J = 7.2, 5.0 Hz, 1H), 2.78 (d, J = 4.4 Hz, 3H), 2.13 (s, 3H). | 448.4 | D | |
| I- 270 | 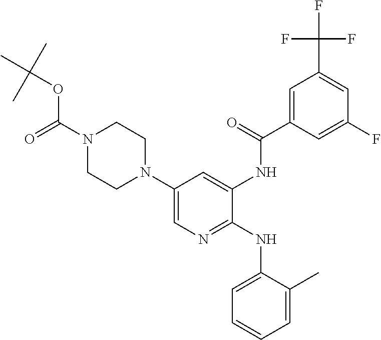 [SMILES: Cc1ccccc1Nc1ncc(N2CCN(C(=O)OC(C)(C)C)CC2)cc1NC(=O)c1cc(F)cc(C(F)(F)F)c1] | (400 MHz, cd3od) 7.98 (s, 1H), 7.85 (d, J = 8.4 Hz, 1H), 7.80 (d, J = 2.8 Hz, 1H), 7.69 (d, J = 8.4 Hz, 1H), 7.60 (d, J = 2.8 Hz, 1H), 7.14 (d, J = 7.4 Hz, 1H), 7.10 (dd, J = 8.0, 1.4 Hz, 1H), 7.08- 7.02 (m, 1H), 6.89 (td, J = 7.3, 1.4 Hz, 1H), 3.67-3.51 (m, 4H), 3.13-3.05 (m, 4H), 2.22 (s, 3H), 1.48 (s, 9H). | 574.53 | D | |
| I- 271 | 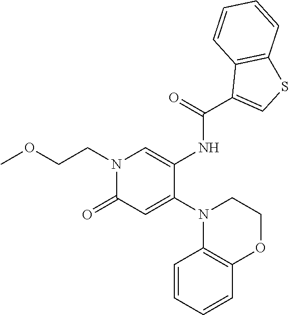 [SMILES: COCCn1cc(NC(=O)c2csc3ccccc23)c(N2CCOc3ccccc32)cc1=O] | (400 MHz, DMSO-d6) 9.42-9.79 (m, 1H), 8.13-8.31 (m, 1H), 7.92-8.07 (m, 1H), 7.77 (s, 1H), 7.59-7.72 (m, 1H), 7.26-7.50 (m, 2H), 6.57-6.84 (m, 3H), 6.10 (s, 1H), 4.17-4.39 (m, 2H), 4.07 (br t, J = 5.13 Hz, 2H), 3.48-3.71 (m, 4H), 2.23-2.65 (m, 3H) | 462 | D | |
| I- 272 | 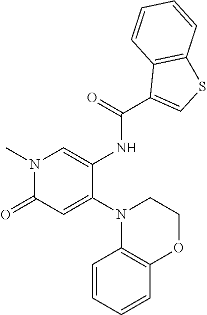 [SMILES: Cn1cc(NC(=O)c2csc3ccccc23)c(N2CCOc3ccccc32)cc1=O] | (400 MHz, DMSO-d6) 9.44-9.72 (m, 1H), 8.32-8.54 (m, 1H), 8.13-8.30 (m, 1H), 7.96-8.05 (m, 1H), 7.77-7.92 (m, 1H), 7.53-7.70 (m, 1H), 7.25-7.48 (m, 1H), 6.55-6.84 (m, 3H), 5.93-6.22 (m, 1H), 4.04-4.39 (m, 2H), 3.49-3.75 (m, 2H), 3.39-3.47 (m, 2H) | 418 | D | |
| I- 273 | 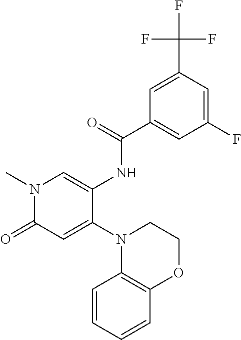 [SMILES: Cn1cc(NC(=O)c2cc(F)cc(C(F)(F)F)c2)c(N2CCOc3ccccc32)cc1=O] | (400 MHz, DMSO-d6) 9.78-9.91 (m, 1H), 7.75-7.93 (m, 2H), 7.34-7.57 (m, 2H), 6.62-6.77 (m, 3H), 6.49-6.60 (m, 1H), 6.07-6.25 (m, 1H), 4.17-4.34 (m, 2H), 3.51-3.76 (m, 2H), 3.40-3.46 (m, 3H) | 448 | D | |
| I- 274 | 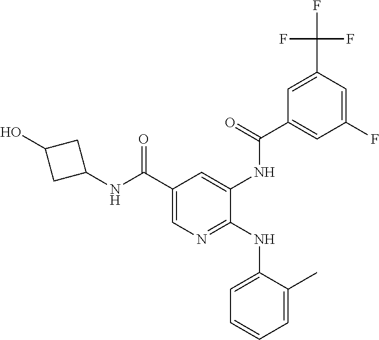 [SMILES: Cc1ccccc1Nc1ncc(C(=O)NC2CC(O)C2)cc1NC(=O)c1cc(F)cc(C(F)(F)F)c1] | | 503.27 | B | B |
| I- 275 | 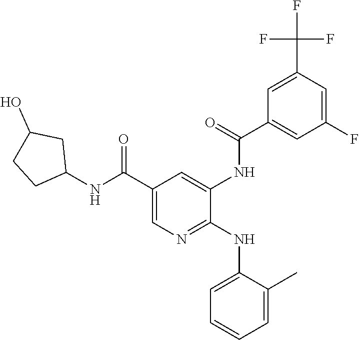 [SMILES: Cc1ccccc1Nc1ncc(C(=O)NC2CCC(O)C2)cc1NC(=O)c1cc(F)cc(C(F)(F)F)c1] | | 517.32 | B | B |
| I- 276 | 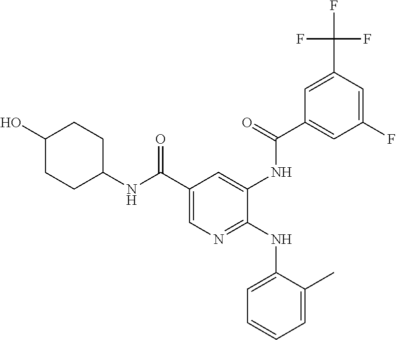 [SMILES: Cc1ccccc1Nc1ncc(C(=O)NC2CCC(O)CC2)cc1NC(=O)c1cc(F)cc(C(F)(F)F)c1] | | 531.33 | B | B |
| I- 277 | 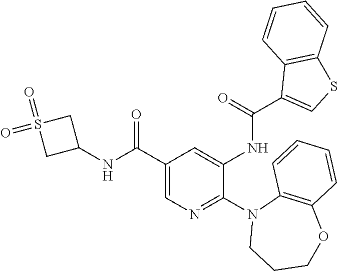 [SMILES: O=C(NC1CS(=O)(=O)C1)c1cnc(N2CCCOc3ccccc32)c(NC(=O)c2csc3ccccc23)c1] | | 549 | E | |
| I- 278 | 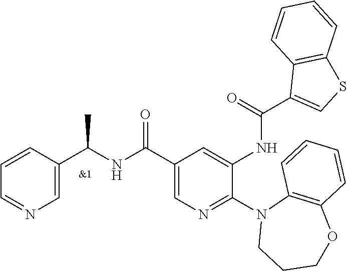 [SMILES: C[C@@H](NC(=O)c1cnc(N2CCCOc3ccccc32)c(NC(=O)c2csc3ccccc23)c1)c1cccnc1] | | 550.2 | D | |
| I- 279 | 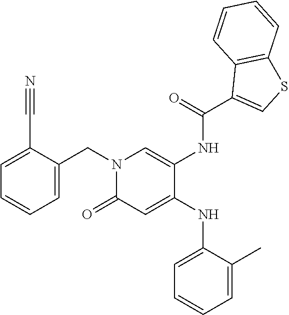 [SMILES: Cc1ccccc1Nc1cc(=O)n(Cc2ccccc2C#N)cc1NC(=O)c1csc2ccccc12] | (400 MHz, DMSO-d6) 9.48-9.79 (m, 1H), 8.56-8.72 (m, 1H), 8.44-8.53 (m, 1H), 8.27-8.40 (m, 1H), 7.94-8.12 (m, 1H), 7.61-7.92 (m, 2H), 7.37-7.54 (m, 2H), 6.95-6.96 (m, 1H), 6.94-7.36 (m, 3H), 6.54-6.82 (m, 1H), 5.19 (s, 3H), 4.55-5.03 (m, 1H), 1.76-2.39 (m, 2H) | 491 | B | D |
| I- 280 | 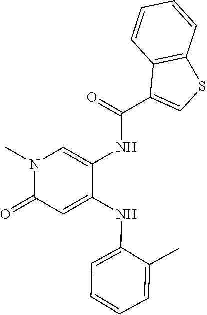 [SMILES: Cc1ccccc1Nc1cc(=O)n(C)cc1NC(=O)c1csc2ccccc12] | (400 MHz, DMSO-d6) 9.60 (s, 1H), 8.62 (s, 1H), 8.49 (br d, J = 7.81 Hz, 1H), 8.25 (s, 1H), 7.96-8.14 (m, 1H), 7.78 (s, 1H), 7.63 (s, 1H), 7.39-7.53 (m, 2H), 7.05-7.34 (m, 4H), 4.74-4.95 (m, 1H), 3.27-3.32 (m, 4H), 2.21-2.59 (m, 3H), 1.96-2.31 (m, 3H), 1.81-2.28 (m, 3H), 1.76-2.28 (m, 3H) | 390 | D | |
| I- 281 | 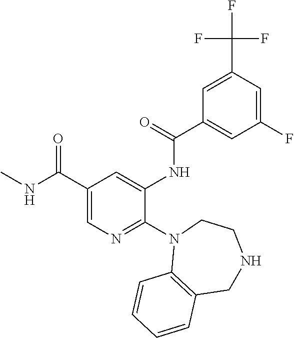 [SMILES: CNC(=O)c1cnc(N2CCNCc3ccccc32)c(NC(=O)c2cc(F)cc(C(F)(F)F)c2)c1] | | 488 | E | |
| I- 282 | 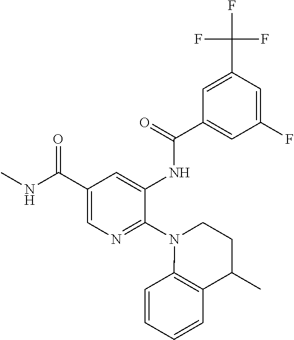 [SMILES: CNC(=O)c1cnc(N2CCC(C)c3ccccc32)c(NC(=O)c2cc(F)cc(C(F)(F)F)c2)c1] | | 487.2 | D | |
| I- 283 | 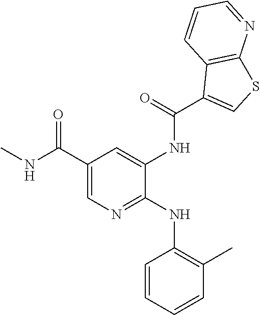 [SMILES: CNC(=O)c1cnc(Nc2ccccc2C)c(NC(=O)c2csc3ncccc23)c1] | | 418.1 | E | |
| I- 284 | 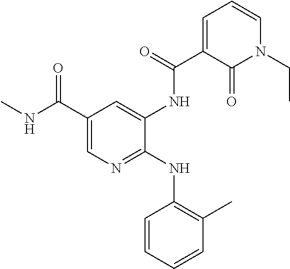 [SMILES: CCn1cccc(C(=O)Nc2cc(C(=O)NC)cnc2Nc2ccccc2C)c1=O] | | 406.1 | E | |
| I- 285 | 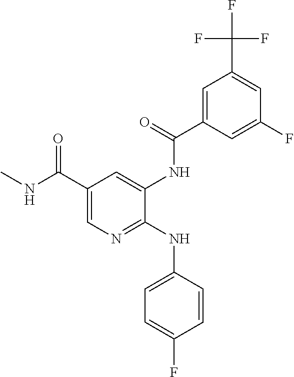 [SMILES: CNC(=O)c1cnc(Nc2ccc(F)cc2)c(NC(=O)c2cc(F)cc(C(F)(F)F)c2)c1] | | | C | D |
| I- 286 | 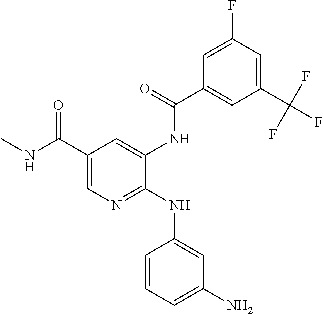 [SMILES: CNC(=O)c1cnc(Nc2cccc(N)c2)c(NC(=O)c2cc(F)cc(C(F)(F)F)c2)c1] | | | D | D |
| I- 287 | 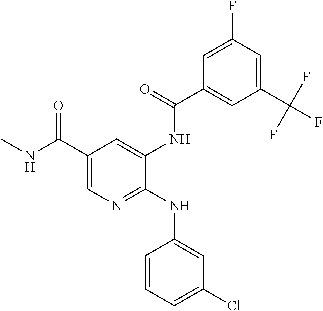 [SMILES: CNC(=O)c1cnc(Nc2cccc(Cl)c2)c(NC(=O)c2cc(F)cc(C(F)(F)F)c2)c1] | | | D | E |
| I- 288 | 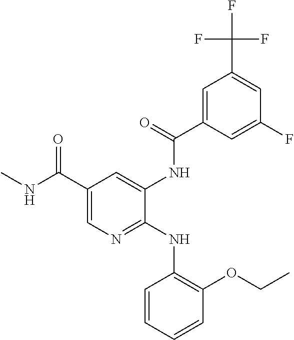 [SMILES: CCOc1ccccc1Nc1ncc(C(=O)NC)cc1NC(=O)c1cc(F)cc(C(F)(F)F)c1] | | | E | E |
| I- 289 | 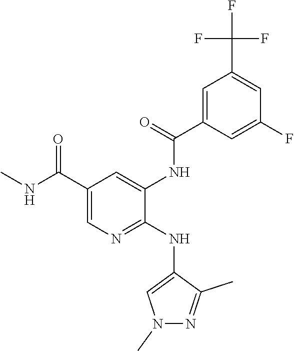 [SMILES: CNC(=O)c1cnc(Nc2cn(C)nc2C)c(NC(=O)c2cc(F)cc(C(F)(F)F)c2)c1] | | | E | E |
| I- 290 | 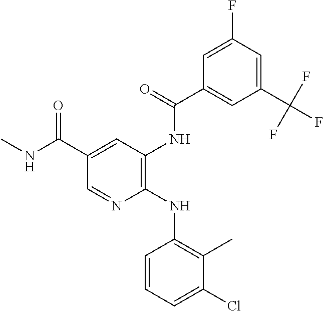 [SMILES: CNC(=O)c1cnc(Nc2cccc(Cl)c2C)c(NC(=O)c2cc(F)cc(C(F)(F)F)c2)c1] | | | D | E |
| I- 291 | 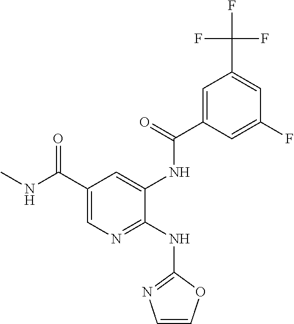 [SMILES: CNC(=O)c1cnc(Nc2ncco2)c(NC(=O)c2cc(F)cc(C(F)(F)F)c2)c1] | | | E | E |
| I- 292 | 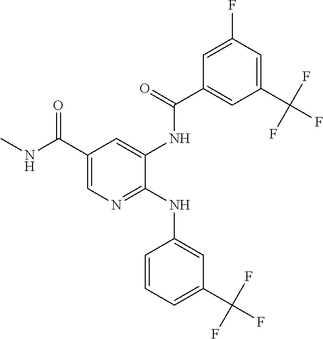 [SMILES: CNC(=O)c1cnc(Nc2cccc(C(F)(F)F)c2)c(NC(=O)c2cc(F)cc(C(F)(F)F)c2)c1] | | | D | D |
| I- 293 | 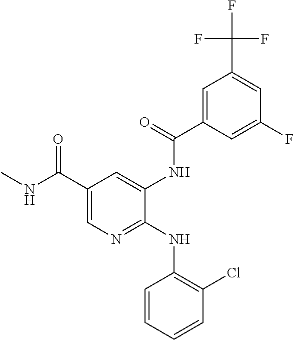 [SMILES: CNC(=O)c1cnc(Nc2ccccc2Cl)c(NC(=O)c2cc(F)cc(C(F)(F)F)c2)c1] | | 467 | B | B |
| I- 294 | 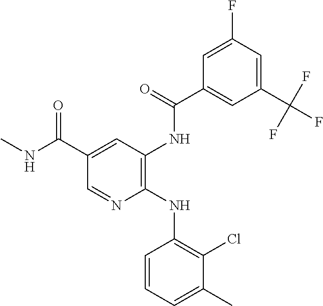 [SMILES: CNC(=O)c1cnc(Nc2cccc(C)c2Cl)c(NC(=O)c2cc(F)cc(C(F)(F)F)c2)c1] | | | D | E |
| I- 295 | 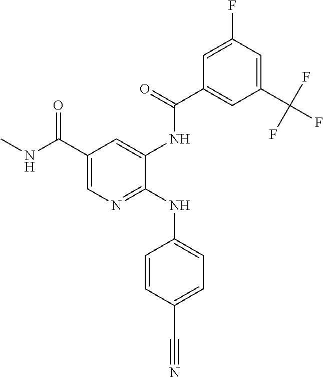 [SMILES: CNC(=O)c1cnc(Nc2ccc(C#N)cc2)c(NC(=O)c2cc(F)cc(C(F)(F)F)c2)c1] | | | D | E |
| I- 296 | 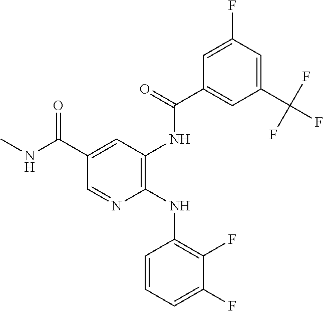 [SMILES: CNC(=O)c1cnc(Nc2cccc(F)c2F)c(NC(=O)c2cc(F)cc(C(F)(F)F)c2)c1] | | | B | C |
| I- 297 | 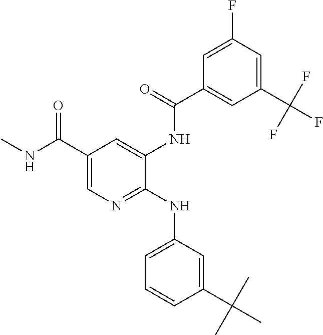 [SMILES: CNC(=O)c1cnc(Nc2cccc(C(C)(C)C)c2)c(NC(=O)c2cc(F)cc(C(F)(F)F)c2)c1] | | | D | E |
| I- 298 | 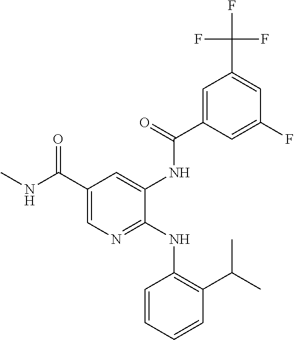 [SMILES: CNC(=O)c1cnc(Nc2ccccc2C(C)C)c(NC(=O)c2cc(F)cc(C(F)(F)F)c2)c1] | | | D | E |
| I- 299 | 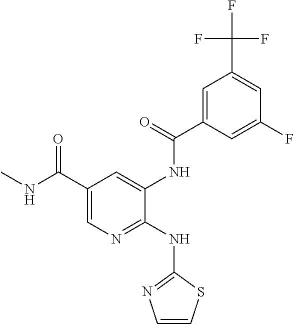 [SMILES: CNC(=O)c1cnc(Nc2nccs2)c(NC(=O)c2cc(F)cc(C(F)(F)F)c2)c1] | | | E | E |
| I- 300 | 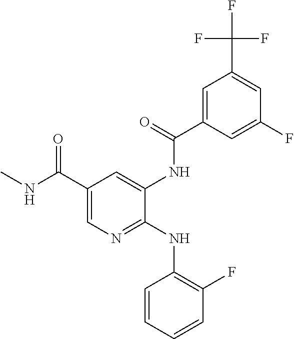 [SMILES: CNC(=O)c1cnc(Nc2ccccc2F)c(NC(=O)c2cc(F)cc(C(F)(F)F)c2)c1] | | | D | E |
| I- 301 | 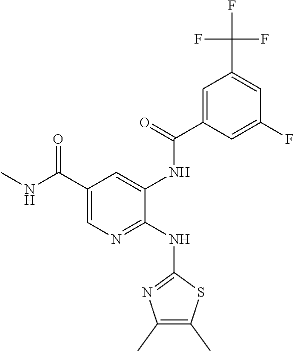 [SMILES: CNC(=O)c1cnc(Nc2nc(C)c(C)s2)c(NC(=O)c2cc(F)cc(C(F)(F)F)c2)c1] | | | C | D |
| I- 302 | 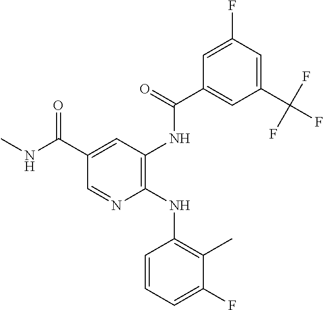 [SMILES: CNC(=O)c1cnc(Nc2cccc(F)c2C)c(NC(=O)c2cc(F)cc(C(F)(F)F)c2)c1] | | | C | C |
| I- 303 | 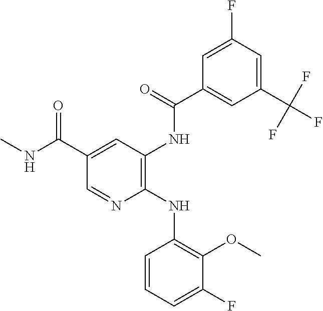 [SMILES: CNC(=O)c1cnc(Nc2cccc(F)c2OC)c(NC(=O)c2cc(F)cc(C(F)(F)F)c2)c1] | | | D | E |
| I- 304 | 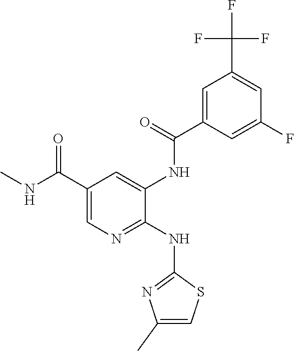 [SMILES: CNC(=O)c1cnc(Nc2nc(C)cs2)c(NC(=O)c2cc(F)cc(C(F)(F)F)c2)c1] | | | D | E |
| I- 305 | 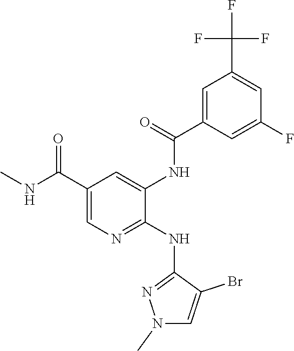 [SMILES: CNC(=O)c1cnc(Nc2nn(C)cc2Br)c(NC(=O)c2cc(F)cc(C(F)(F)F)c2)c1] | | | E | E |
| I- 306 | 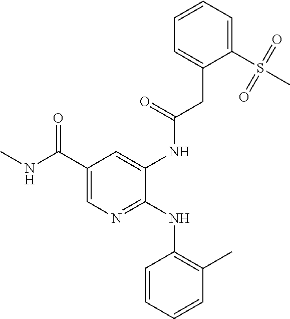 | (400 MHz, dmso) 9.83 (s, 1H), 8.32 (d, J = 2.0 Hz, 1H), 8.23 (q, J = 4.4 Hz, 1H), 7.93 (d, J = 6.9 Hz, 2H), 7.85 (s, 1H), 7.68 (t, J = 6.9 Hz, 1H), 7.57- 7.52 (m, 2H), 7.43 (s, 1H), 7.19 (d, J = 7.6 Hz, 1H), 7.15 (t, J = 7.6 Hz, 1H), 7.02 (t, J = 7.6 Hz, 1H), 4.24 (s, 2H), 3.22 (s, 3H), 2.71 (d, J = 4.4 Hz, 3H), 2.11 (s, 3H). | | E | |
| I- 307 | 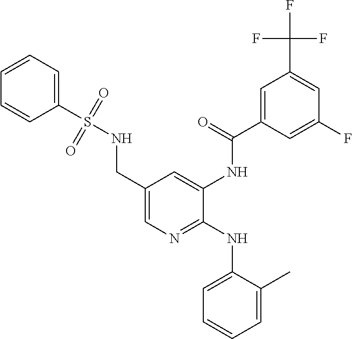 [SMILES: Cc1ccccc1Nc1ncc(CNS(=O)(=O)c2ccccc2)cc1NC(=O)c1cc(F)cc(C(F)(F)F)c1] | (400 MHz, dmso) 10.26 (s, 1H), 8.20 (s, 1H), 8.13 (dd, J = 12.4, 6.4 Hz, 2H), 7.98 (d, J = 8.4 Hz, 1H), 7.81 (ddd, J = 12.5, 6.9, 1.7 Hz, 3H), 7.74 (s, 1H), 7.65-7.56 (m, 3H), 7.53 (d, J = 2.1 Hz, 1H), 7.33 (dd, J = 8.1, 0.8 Hz, 1H), 7.17 (d, J = 7.9 Hz, 1H), 7.13 (td, J = 7.7, 1.5 Hz, 1H), 6.99 (td, J = 7.4, 1.3 Hz, 1H), 3.90 (d, J = 6.2 Hz, 2H), 2.13 (s, 3H). | | B | B |
| I- 308 | 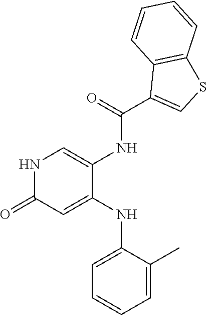 [SMILES: Cc1ccccc1Nc1cc(=O)[nH]cc1NC(=O)c1csc2ccccc12] | | 376 | D | E |
| I- 309 | 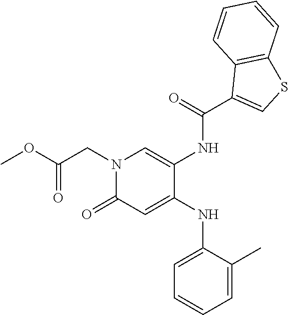 [SMILES: COC(=O)Cn1cc(NC(=O)c2csc3ccccc23)c(Nc2ccccc2C)cc1=O] | (400 MHz, DMSO-d6) 9.64 (s, 1H), 8.62 (s, 1H), 8.45-8.56 (m, 1H), 8.13 (s, 3H), 8.06 (dd, J = 1.01, 7.07 Hz, 1H), 7.95 (s, 1H), 7.64 (s, 1H), 7.36-7.53 (m, 2H), 7.17-7.35 (m, 3H), 7.14 (d, J = 7.83 Hz, 1H), 4.85 (s, 1H), 4.57 (s, 2H), 3.65 (s, 4H), 2.46-2.51 (m, 28H) | 448 | D | E |
| I- 310 | 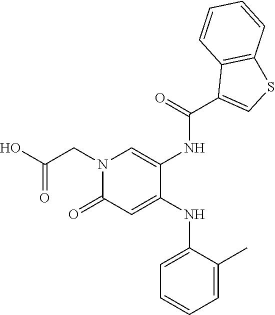 [SMILES: Cc1ccccc1Nc1cc(=O)n(CC(=O)O)cc1NC(=O)c1csc2ccccc12] | (400 MHz, DMSO-d6) 9.57-9.75 (m, 1H), 8.56-8.70 (m, 1H), 8.41-8.55 (m, 1H), 7.98-8.11 (m, 1H), 7.81-7.92 (m, 1H), 7.53-7.64 (m, 1H), 7.38-7.50 (m, 2H), 7.28-7.34 (m, 1H), 7.17-7.28 (m, 2H), 7.07-7.16 (m, 1H), 4.86 (s, 1H), 4.15-4.52 (m, 2H) | 434 | E | E |
| I- 311 | 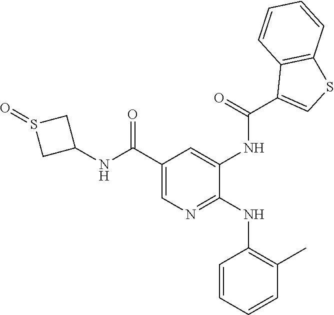 [SMILES: Cc1ccccc1Nc1ncc(C(=O)NC2CS(=O)C2)cc1NC(=O)c1csc2ccccc12] | | 491.4 | A | B |
| I- 312 | 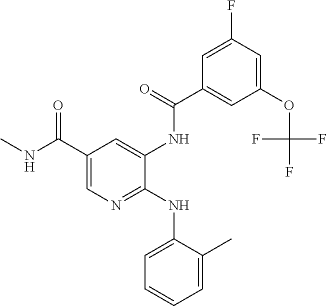 [SMILES: CNC(=O)c1cnc(Nc2ccccc2C)c(NC(=O)c2cc(F)cc(OC(F)(F)F)c2)c1] | | 463.41 | D | E |
| I- 313 | 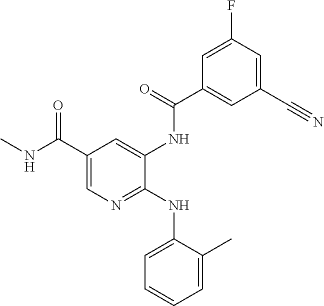 [SMILES: CNC(=O)c1cnc(Nc2ccccc2C)c(NC(=O)c2cc(F)cc(C#N)c2)c1] | | 404.4 | D | D |
| I- 314 | 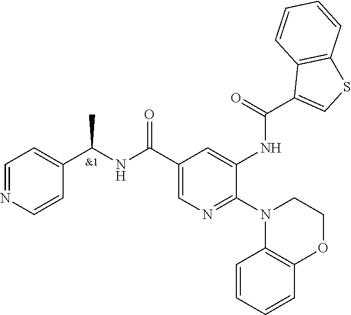 [SMILES: C[C@@H](NC(=O)c1cnc(N2CCOc3ccccc32)c(NC(=O)c2csc3ccccc23)c1)c1ccncc1] | | 536.5 | B | C |
| I- 315 | 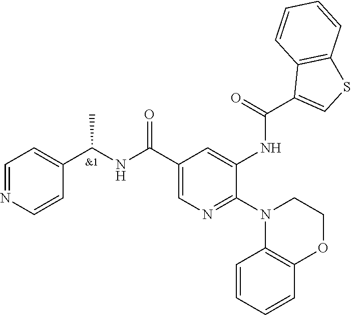 [SMILES: C[C@H](NC(=O)c1cnc(N2CCOc3ccccc32)c(NC(=O)c2csc3ccccc23)c1)c1ccncc1] | | 536.4 | C | D |
| I- 316 | 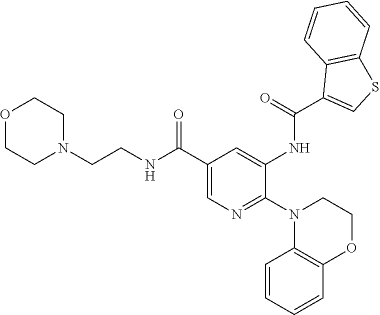 [SMILES: O=C(NCCN1CCOCC1)c1cnc(N2CCOc3ccccc32)c(NC(=O)c2csc3ccccc23)c1] | | | D | E |
| I- 317 | 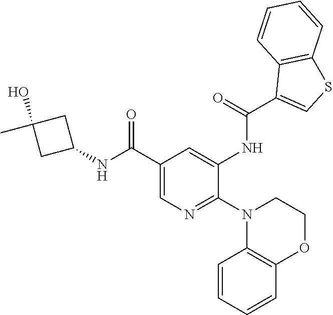 [SMILES: C[C@]1(O)C[C@@H](NC(=O)c2cnc(N3CCOc4ccccc43)c(NC(=O)c3csc4ccccc34)c2)C1] | | | B | C |
| I- 318 | 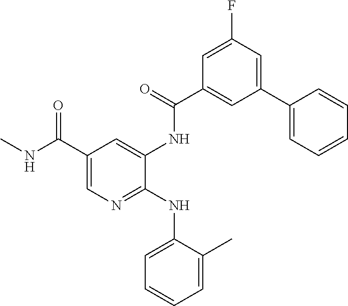 [SMILES: CNC(=O)c1cnc(Nc2ccccc2C)c(NC(=O)c2cc(F)cc(-c3ccccc3)c2)c1] | | 455.1 | E | |
| I- 319 | 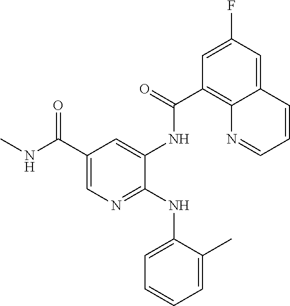 [SMILES: CNC(=O)c1cnc(Nc2ccccc2C)c(NC(=O)c2cc(F)cc3cccnc23)c1] | | 430.4 | E | |
| I- 320 | 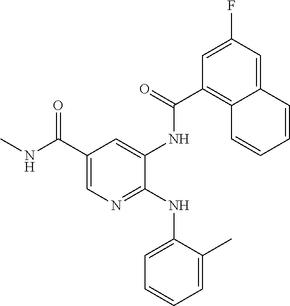 [SMILES: CNC(=O)c1cnc(Nc2ccccc2C)c(NC(=O)c2cc(F)cc3ccccc23)c1] | | 429.45 | E | |
| I- 321 | 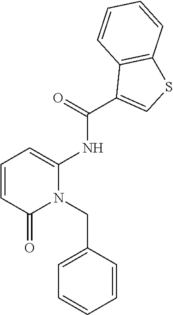 [SMILES: O=C(Nc1cccc(=O)n1Cc1ccccc1)c1csc2ccccc12] | | 361.24 | E | |
| I- 322 | 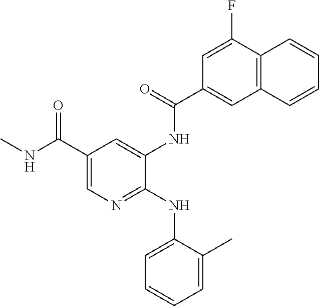 [SMILES: CNC(=O)c1cnc(Nc2ccccc2C)c(NC(=O)c2cc(F)c3ccccc3c2)c1] | | 429.06 | E | |
| I- 323 | 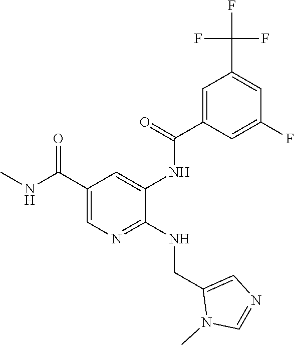 [SMILES: CNC(=O)c1cnc(NCc2cncn2C)c(NC(=O)c2cc(F)cc(C(F)(F)F)c2)c1] | (400 MHz, dmso) 10.09 (s, 1H), 8.52 (d, J = 2.2 Hz, 1H), 8.24 (q, J = 4.3 Hz, 1H), 8.20 (s, 1H), 8.15 (d, J = 8.4 Hz, 1H), 7.98 (d, J = 8.4 Hz, 1H), 7.87 (d, J = 2.2 Hz, 1H), 7.53 (br s, 1H), 7.20- 7.16 (m, 1H), 6.82 (s, 1H), 4.59 (d, J = 5.3 Hz, 2H), 3.61 (s, 3H), 2.75 (d, J = 4.5 Hz, 3H). | 451.34 | E | |
| I- 324 | 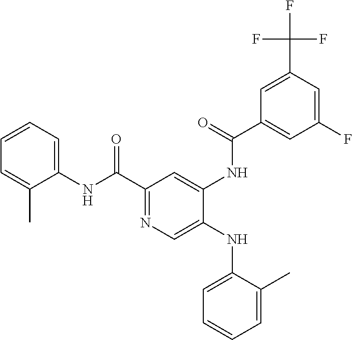 [SMILES: Cc1ccccc1NC(=O)c1cc(NC(=O)c2cc(F)cc(C(F)(F)F)c2)c(Nc2ccccc2C)cn1] | (400 MHz, dmso) 10.40 (s, 1H), 10.01 (s, 1H), 8.29 (s, 1H), 8.07 (s, 1H), 8.04 (d, J = 9.8 Hz, 1H), 8.02 (s, 1H), 7.98 (d, J = 8.5 Hz, 1H), 7.90 (d, J = 7.1 Hz, 1H), 7.77 (s, 1H), 7.30-7.13 (m, 4H), 7.12-7.02 (m, 3H), 2.29 (s, 3H), 2.25 (s, 3H). | 523.36 | E | |
| I- 325 | 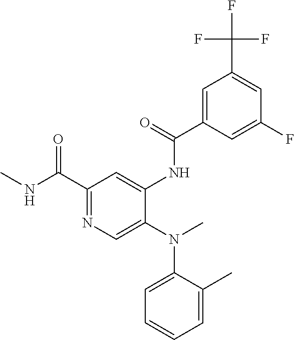 [SMILES: CNC(=O)c1cc(NC(=O)c2cc(F)cc(C(F)(F)F)c2)c(N(C)c2ccccc2C)cn1] | (400 MHz, cdcl3) 8.75 (s, 1H), 8.52 (s, 1H), 7.45 (d, J = 7.7 Hz, 1H), 7.23-7.03 (m, 6H), 6.49-6.43 (m, 1H), 3.50 (s, 3H), 3.02 (d, J = 4.1 Hz, 3H), 1.26 (s, 3H). | 461.36 | D | |
| I- 326 | 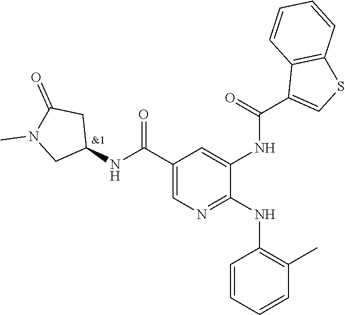 [SMILES: Cc1ccccc1Nc1ncc(C(=O)N[C@@H]2CC(=O)N(C)C2)cc1NC(=O)c1csc2ccccc12] | (400 MHz, dmso) 10.03 (s, 1H), 8.67 (s, 1H), 8.59 (d, J = 6.6 Hz, 1H), 8.48- 8.45 (m, 1H), 8.44 (d, J = 2.2 Hz, 1H), 8.22 (s, 1H), 8.11 (d, J = 2.2 Hz, 1H), 8.06 (dd, J = 7.6, 1.3 Hz, 1H), 7.50- 7.36 (m, 3H), 7.19 (d, J = 7.6 Hz, 1H), 7.14 (td, J = 7.6, 1.3 Hz, 1H), 7.03 (td, J = 7.6, 1.3 Hz, 1H), 4.59-4.42 (m, 1H), 3.66 (dd, J = 10.2, 8.1 Hz, 1H), 3.22 (dd, J = 10.2, 4.3 Hz, 1H), 2.70 (s, 3H), 2.60 (dd, J = 17.0, 8.1 Hz, 1H), 2.29 (dd, J = 17.0, 4.3 Hz, 1H), 2.14 (s, 3H). | 500.33 | B | C |
| I- 327 | 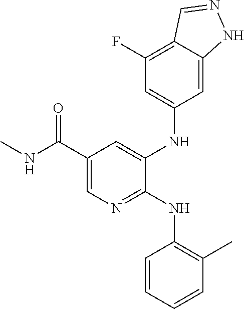 | (400 MHz, dmso) 12.75 (s, 1H), 8.34 (d, J = 2.0 Hz, 1H), 8.26 (q, J = 4.5 Hz, 1H), 7.95-7.89 (m, 3H), 7.83 (s, 1H), 7.61 (d, J = 7.8 Hz, 1H), 7.14 (t, J = 7.8 Hz, 2H), 6.99 (td, J = 7.8, 1.5 Hz, 1H), 6.51 (dd, J = 12.2, 1.5 Hz, 1H), 6.44 (t, J = 1.5 Hz, 1H), 2.70 (d, J = 4.5 Hz, 3H), 2.03 (s, 3H). | 391.34 | E | |
| I- 328 | 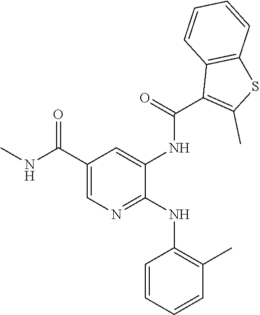 [SMILES: CNC(=O)c1cnc(Nc2ccccc2C)c(NC(=O)c2c(C)sc3ccccc23)c1] | (400 MHz, dmso) 10.06 (s, 1H), 8.44 (d, J = 1.9 Hz, 1H), 8.36 (dd, J = 9.2, 4.8 Hz, 1H), 8.25 (s, 1H), 8.01-7.87 (m, 3H), 7.49 (d, J = 7.7 Hz, 1H), 7.46- 7.35 (m, 2H), 7.23 (d, J = 7.6 Hz, 1H), 7.18 (td, J = 7.7, 1.4 Hz, 1H), 7.05 (td, J = 7.4, 1.2 Hz, 1H), 2.78 (d, J = 4.4 Hz, 3H), 2.76 (s, 3H), 2.18 (s, 3H). | 431.33 | E | |
| I- 329 | 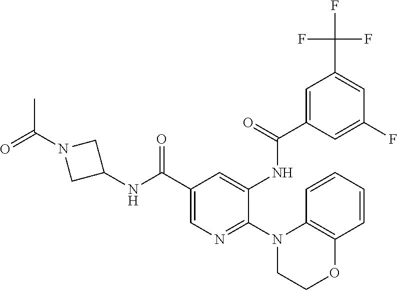 [SMILES: CC(=O)N1CC(NC(=O)c2cnc(N3CCOc4ccccc43)c(NC(=O)c3cc(F)cc(C(F)(F)F)c3)c2)C1] | (400 MHz, dmso) 10.24 (s, 1H), 9.14 (d, J = 6.8 Hz, 1H), 8.80 (d, J = 2.2 Hz, 1H), 8.28 (d, J = 2.2 Hz, 1H), 7.86 (d, J = 8.6 Hz, 1H), 7.48-7.39 (m, 2H), 6.70 (m, 2H), 6.63-6.56 (m, 1H), 6.45 (dd, J = 8.0, 1.4 Hz, 1H), 4.70 (m, 1H), 4.43 (m, 3H), 4.14 (t, J = 9.0 Hz, 1H), 4.08 (dd, J = 8.8, 5.3 Hz, 1H), 3.92 (t, J = 4.2 Hz, 2H), 3.87 (dd, J = 9.9, 5.5 Hz, 1H), 1.78 (s, 3H) | 558.4 | C | D |
| I- 330 | 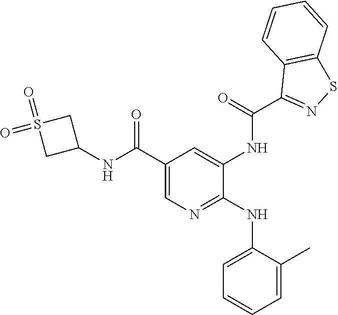 [SMILES: Cc1ccccc1Nc1ncc(C(=O)NC2CS(=O)(=O)C2)cc1NC(=O)c1nsc2ccccc12] | | 508.5 | B | B |
| I- 331 | 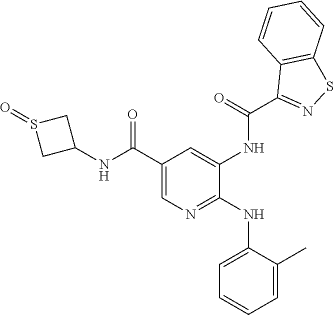 [SMILES: Cc1ccccc1Nc1ncc(C(=O)NC2CS(=O)C2)cc1NC(=O)c1nsc2ccccc12] | | 492.4 | B | B |
| I- 332 | 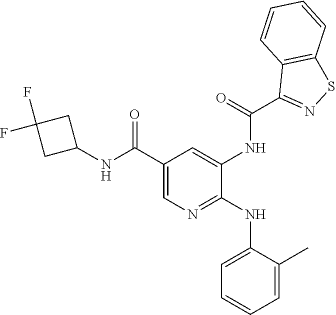 [SMILES: Cc1ccccc1Nc1ncc(C(=O)NC2CC(F)(F)C2)cc1NC(=O)c1nsc2ccccc12] | | 494.5 | B | C |
| I- 333 | 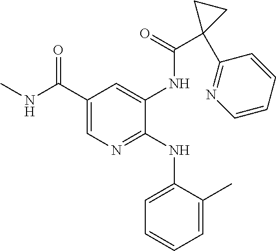 [SMILES: CNC(=O)c1cnc(Nc2ccccc2C)c(NC(=O)C2(c3ccccn3)CC2)c1] | | 402.4 | E | |
| I- 334 | 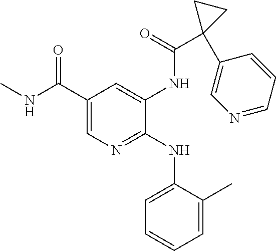 [SMILES: CNC(=O)c1cnc(Nc2ccccc2C)c(NC(=O)C2(c3cccnc3)CC2)c1] | | 402.4 | E | |
| I- 335 | 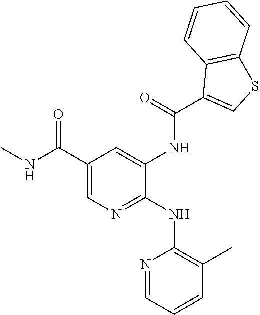 [SMILES: CNC(=O)c1cnc(Nc2ncccc2C)c(NC(=O)c2csc3ccccc23)c1] | | 418.4 | C | D |
| I- 336 | 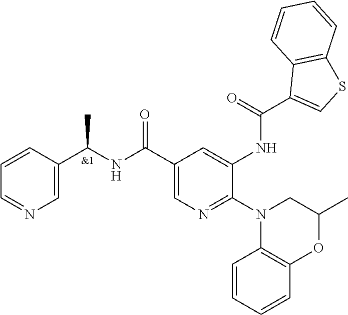 [SMILES: CC1CN(c2ncc(C(=O)N[C@H](C)c3cccnc3)cc2NC(=O)c2csc3ccccc23)c2ccccc2O1] | | 550.5 | D | |
| I- 337 | 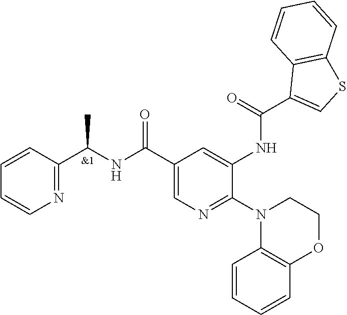 [SMILES: C[C@@H](NC(=O)c1cnc(N2CCOc3ccccc32)c(NC(=O)c2csc3ccccc23)c1)c1ccccn1] | | 536.4 | B | C |
| I- 338 | 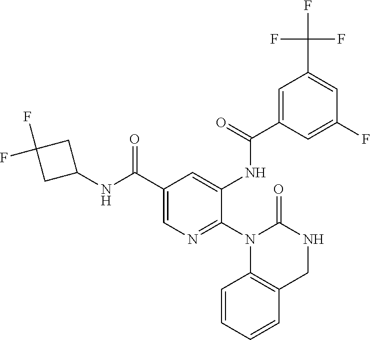 [SMILES: O=C(Nc1cc(C(=O)NC2CC(F)(F)C2)cnc1N1C(=O)NCc2ccccc21)c1cc(F)cc(C(F)(F)F)c1] | | 564.4 | D | |
| I- 339 | 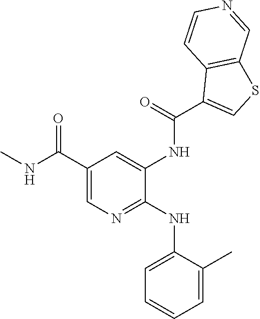 [SMILES: CNC(=O)c1cnc(Nc2ccccc2C)c(NC(=O)c2csc3cnccc23)c1] | | | C | B |
| I- 340 | 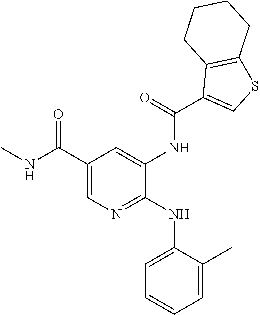 [SMILES: CNC(=O)c1cnc(Nc2ccccc2C)c(NC(=O)c2csc3c2CCCC3)c1] | | | D | |
| I- 341 | 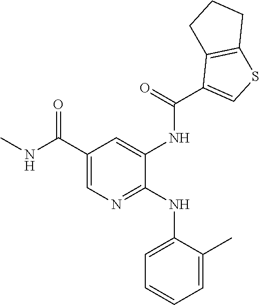 [SMILES: CNC(=O)c1cnc(Nc2ccccc2C)c(NC(=O)c2csc3c2CCC3)c1] | | | D | |
| I- 342 | 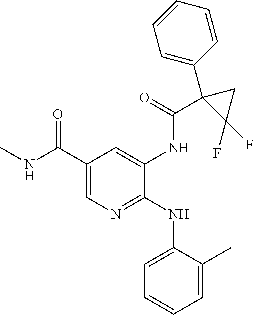 [SMILES: CNC(=O)c1cnc(Nc2ccccc2C)c(NC(=O)C2(c3ccccc3)CC2(F)F)c1] | | | E | |
| I- 343 | 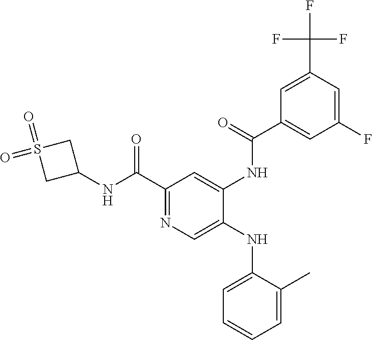 [SMILES: Cc1ccccc1Nc1cnc(C(=O)NC2CS(=O)(=O)C2)cc1NC(=O)c1cc(F)cc(C(F)(F)F)c1] | (400 MHz, dmso) 10.35 (s, 1H), 8.91 (d, J = 7.4 Hz, 1H), 8.19 (s, 1H), 8.02 (s, 2H), 7.97 (d, J = 8.6 Hz, 2H), 7.69 (s, 1H), 7.24 (d, J = 7.6 Hz, 1H), 7.13 (t, J = 7.5 Hz, 1H), 7.04-6.94 (m, 2H), 5.12-5.08 (m, 1H), 4.74 (dd, J = 9.2, 5.7 Hz, 1H), 4.50 (dd, J = 9.2, 3.9 Hz, 1H), 3.73 (dd, J = 13.4, 5.5 Hz, 1H), 3.23-3.08 (m, 1H), 2.22 (s, 3H). Contains peak of DCM. | 537.28 | D | A |
| I- 344 | 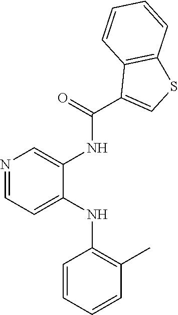 [SMILES: Cc1ccccc1Nc1ccncc1NC(=O)c1csc2ccccc12] | (400 MHz, dmso) 9.94 (s, 1H), 8.62 (s, 1H), 8.45 (d, J = 7.1 Hz, 1H), 8.24 (s, 1H), 8.05 (dd, J = 7.1, 1.3 Hz, 1H), 8.00 (d, J = 5.6 Hz, 1H), 7.76 (s, 1H), 7.49-7.39 (m, 2H), 7.27 (d, J = 7.1 Hz, 1H), 7.25-7.18 (m, 1H), 7.18-7.07 (m, 2H), 6.37 (d, J = 5.6 Hz, 1H), 2.16 (s, 3H). | 360.28 | D | |
| I- 345 | 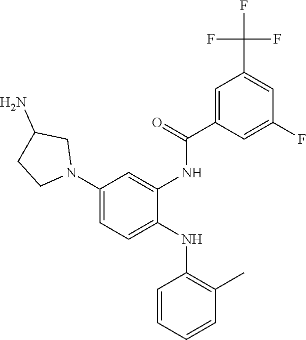 [SMILES: Cc1ccccc1Nc1ccc(N2CCC(N)C2)cc1NC(=O)c1cc(F)cc(C(F)(F)F)c1] | (400 MHz, cd3od) 8.50 (br s, 1H), 7.86 (s, 1H), 7.74 (d, J = 8.9 Hz, 1H), 7.68 (d, J = 8.3 Hz, 1H), 7.59 (d, J = 2.7 Hz, 1H), 7.45 (d, J = 2.8 Hz, 1H), 7.13 (d, J = 7.4 Hz, 1H), 6.99 (ddd, J = 8.3, 7.5, 1.1 Hz, 1H), 6.87 (dd, J = 8.0, 1.1 Hz, 1H), 6.82 (td, J = 7.4, 1.2 Hz, 1H), 4.08-3.97 (m, 1H), 3.66-3.60 (m, 1H), 3.59 (t, J = 5.3 Hz, 1H), 3.44 (dd, J = 10.6, 3.1 Hz, 1H), 3.37 (td, J = 9.1, 5.1 Hz, 1H), 2.49 (ddt, J = 13.6, 8.9, 6.7 Hz, 1H), 2.24 (s, 3H), 2.20-2.11 (m, 1H). | 474.37 | D | |
| I- 346 | 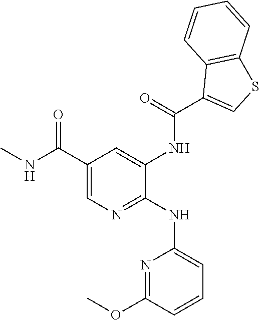 [SMILES: CNC(=O)c1cnc(Nc2cccc(OC)n2)c(NC(=O)c2csc3ccccc23)c1] | | 434.3 | D | |
| I- 347 | 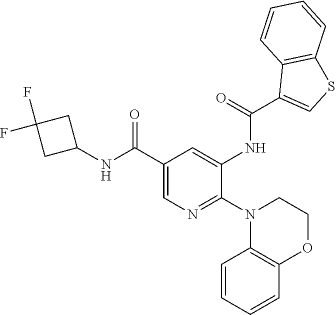 [SMILES: O=C(NC1CC(F)(F)C1)c1cnc(N2CCOc3ccccc32)c(NC(=O)c2csc3ccccc23)c1] | | 521.4 | B | B |
| I- 348 | 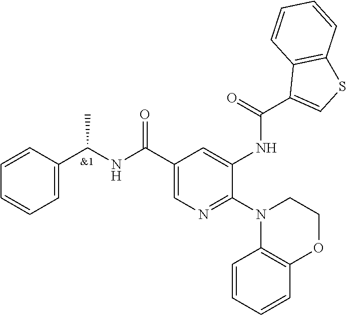 [SMILES: C[C@H](NC(=O)c1cnc(N2CCOc3ccccc32)c(NC(=O)c2csc3ccccc23)c1)c1ccccc1] | | 536.4 | B | C |
| I- 349 | 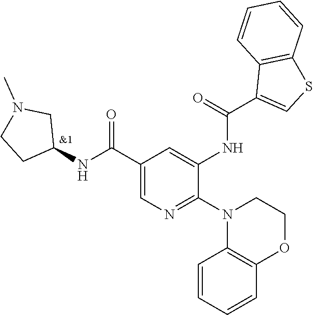 [SMILES: CN1CC[C@H](NC(=O)c2cnc(N3CCOc4ccccc43)c(NC(=O)c3csc4ccccc34)c2)C1] | | 514.5 | C | |
| I- 350 | 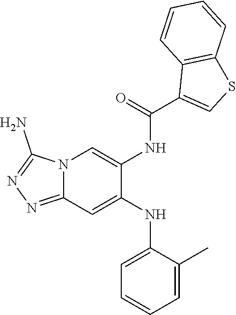 [SMILES: Cc1ccccc1Nc1cc2nnc(N)n2cc1NC(=O)c1csc2ccccc12] | (400 MHz, DMSO-d6) Shift 9.83- 10.03 (m, 1H), 8.61-8.72 (m, 1H), 8.44-8.55 (m, 2H), 8.24-8.39 (m, 1H), 8.02-8.16 (m, 1H), 7.63-7.79 (m, 1H), 7.40-7.56 (m, 2H), 7.30-7.37 (m, 1H), 7.23-7.30 (m, 1H), 7.10-7.22 (m, 2H), 5.86-6.04 (m, 1H), 5.62-5.77 (m, 2H), 2.09-2.34 (m, 3H) | 415 | D | B |
| I- 351 | 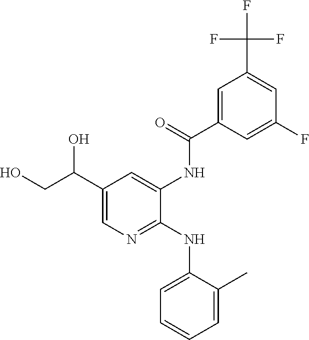 [SMILES: Cc1ccccc1Nc1ncc(C(O)CO)cc1NC(=O)c1cc(F)cc(C(F)(F)F)c1] | | 450.3 | D | |
| I- 352 | 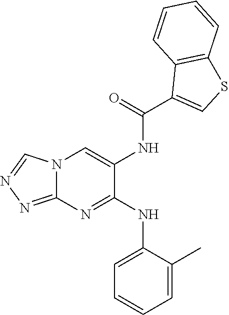 [SMILES: Cc1ccccc1Nc1nc2nncn2cc1NC(=O)c1csc2ccccc12] | (400 MHz, DMSO-d6) Shift 9.97- 10.19 (m, 1H), 8.81-9.02 (m, 1H), 8.63-8.77 (m, 1H), 8.48-8.58 (m, 1H), 8.38-8.47 (m, 1H), 8.13-8.23 (m, 1H), 7.93-8.12 (m, 1H), 7.41-7.54 (m, 1H), 7.34-7.40 (m, 1H), 7.27-7.33 (m, 1H), 7.19-7.27 (m, 1H), 6.60-6.78 (m, 1H), 6.14-6.23 (m, 1H), 2.11-2.30 (m, 3H) | 400 | C | B |
| I- 353 | 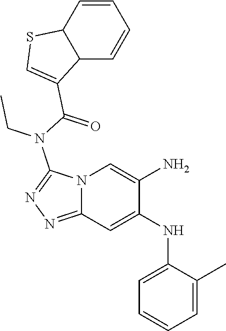 [SMILES: CCN(C(=O)C1=CSC2C=CC=CC12)c1nnc2cc(Nc3ccccc3C)c(N)cn12] | (400 MHz, DMSO-d6) Shift 8.49-8.69 (m, 1H), 8.38 (br s, 1H), 8.19-8.33 (m, 1H), 7.94-8.04 (m, 1H), 7.33-7.54 (m, 2H), 7.06-7.33 (m, 3H), 5.90-6.05 (m, 1H), 5.70-5.87 (m, 1H), 2.01-2.26 (m, 2H), 1.07-1.42 (m, 3H) | 443 | E | |
| I- 354 | 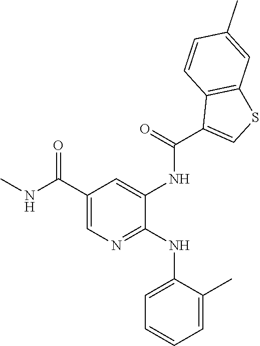 [SMILES: CNC(=O)c1cnc(Nc2ccccc2C)c(NC(=O)c2csc3cc(C)ccc23)c1] | (400 MHz, dmso) 10.02 (s, 1H), 8.61 (s, 1H), 8.43 (d, J = 2.2 Hz, 1H), 8.37 (d, J = 8.3 Hz, 1H), 8.32 (q, J = 4.3 Hz, 1H), 8.19 (s, 1H), 8.11 (d, J = 2.2 Hz, 1H), 7.89-7.86 (m, 1H), 7.45 (dd, J = 7.9, 1.0 Hz, 1H), 7.32 (dd, J = 8.6, 1.3 Hz, 1H), 7.22 (d, J = 7.5 Hz, 1H), 7.20- 7.15 (m, 1H), 7.05 (td, J = 7.4, 1.3 Hz, 1H), 2.77 (d, J = 4.5 Hz, 3H), 2.45 (s, 3H), 2.17 (s, 3H). | 431.36 | D | |
| I- 355 | 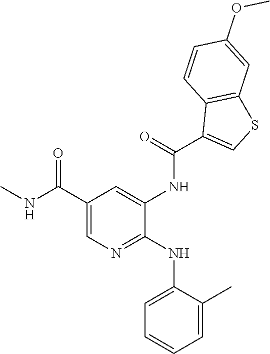 | (400 MHz, dmso) 10.05 (s, 1H), 8.48 (d, J = 0.5 Hz, 1H), 8.40 (d, J = 2.2 Hz, 1H), 8.33 (d, J = 9.0 Hz, 1H), 8.28 (q, J = 4.4 Hz, 1H), 8.16 (s, 1H), 8.06 (d, J = 1.9 Hz, 1H), 7.62 (d, J = 2.4 Hz, 1H), 7.41 (d, J = 7.0 Hz, 1H), 7.19 (d, J = 7.5 Hz, 1H), 7.15 (td, J = 7.6, 1.4 Hz, 1H), 7.08 (dd, J = 9.0, 2.4 Hz, 1H), 7.02 (td, J = 7.4, 1.3 Hz, 1H), 3.81 (s, 3H), 2.75 (d, J = 4.5 Hz, 3H), 2.15 (s, 3H). | 447.33 | E | |
| I- 356 | 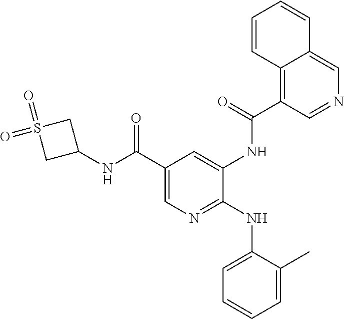 [SMILES: Cc1ccccc1Nc1ncc(C(=O)NC2CS(=O)(=O)C2)cc1NC(=O)c1cncc2ccccc12] | (400 MHz, dmso) 9.42 (d, J = 2.1 Hz, 1H), 9.00 (d, J = 2.1 Hz, 1H), 8.89 (d, J = 3.5 Hz, 1H), 8.46-8.40 (m, 2H), 8.16- 8.07 (m, 3H), 7.88 (ddd, J = 8.1, 6.9, 1.5 Hz, 1H), 7.70 (ddd, J = 8.1, 6.9, 1.2 Hz, 1H), 7.32 (d, J = 8.1 Hz, 1H), 7.21 (d, J = 7.5 Hz, 1H), 7.16 (t, J = 7.5 Hz, 1H), 7.06 (td, J = 7.5, 1.3 Hz, 1H), 4.58-4.47 (m, 3H), 4.27-4.22 (m, 3H), 2.15 (s, 3H). | 502.35 | E | |
| I- 357 | 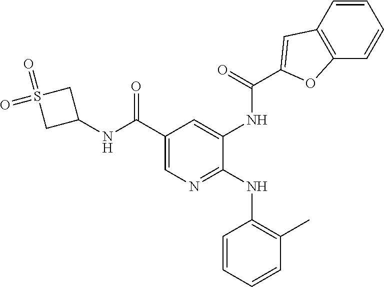 [SMILES: Cc1ccccc1Nc1ncc(C(=O)NC2CS(=O)(=O)C2)cc1NC(=O)c1cc2ccccc2o1] | (400 MHz, dmso) 8.87 (d, J = 3.5 Hz, 1H), 8.42 (d, J = 2.1 Hz, 2H), 8.05 (s, 1H), 7.80 (d, J = 7.5 Hz, 1H), 7.71 (s, 1H), 7.69 (dd, J = 8.4, 1.1 Hz, 1H), 7.47 (ddd, J = 8.4, 7.5, 1.1 Hz, 1H), 7.38-7.28 (m, 2H), 7.19 (d, J = 7.5 Hz, 1H), 7.14 (td, J = 7.5, 1.1 Hz, 1H), 7.04 (td, J = 7.5, 1.1 Hz, 1H), 4.57-4.47 (m, 3H), 4.26-4.20 (m, 2H), 2.13 (s, 3H). | 491.32 | E | |
| I- 358 | 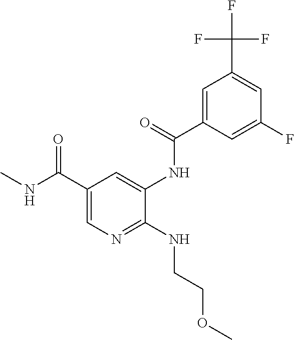 [SMILES: CNC(=O)c1cnc(NCCOC)c(NC(=O)c2cc(F)cc(C(F)(F)F)c2)c1] | (400 MHz, dmso) 10.07 (s, 1H), 8.48 (d, J = 2.2 Hz, 1H), 8.25-8.12 (m, 3H), 7.98 (d, J = 8.5 Hz, 1H), 7.86 (d, J = 2.2 Hz, 1H), 6.91 (t, J = 5.6 Hz, 1H), 3.55 (m, 2H), 3.47 (m, 2H), 3.25 (s, 3H), 2.75 (s) and 2.74 (s) (3H, rotamers). | 415.34 | E | |
| I- 359 | 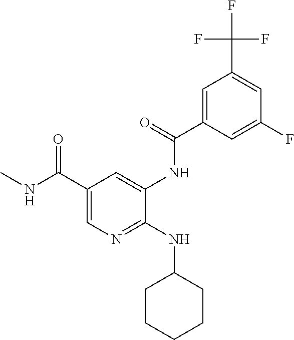 [SMILES: CNC(=O)c1cnc(NC2CCCCC2)c(NC(=O)c2cc(F)cc(C(F)(F)F)c2)c1] | (400 MHz, dmso) 9.90 (s, 1H), 8.46 (d, J = 2.2 Hz, 1H)., 8.17 (m, 3H), 7.99 (d, J = 8.4 Hz, 1H), 7.85 (d, J = 2.2 Hz, 1H)., 6.41 (d, J = 7.8 Hz, 1H), 4.04- 3.90 (m, 1H), 2.75 (s) and 2.74 (s) (3H, rotamers), 1.93-1.84 (m, 2H), 1.77- 1.67 (m, 2H), 1.61 (d, J = 12.8 Hz, 1H), 1.41-1.20 (m, 4H), 1.18-1.04 (m, 1H). | 439.35 | E | |
| I- 360 | 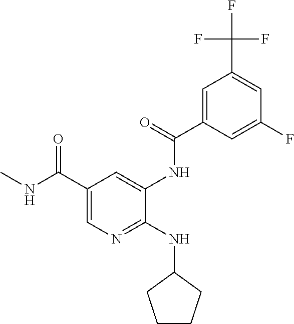 [SMILES: CNC(=O)c1cnc(NC2CCCC2)c(NC(=O)c2cc(F)cc(C(F)(F)F)c2)c1] | (400 MHz, DMSO) 9.97 (s, 1H), 8.47 (d, J = 2.2 Hz, 1H), 8.18 (m, 3H), 7.99 (m, 1H), 7.86 (d, J = 2.1 Hz, 1H), 6.54 (s) and 6.52 (s) (1H, rotamers), 4.38 (m, 1H), 2.75 (s) and 2.74 (s) (3H, rotamers), 1.95 (m, 2H), 1.76-1.61 (m, 2H), 1.58-1.42 (m, 4H). | 425.35 | E | |
| I- 361 | 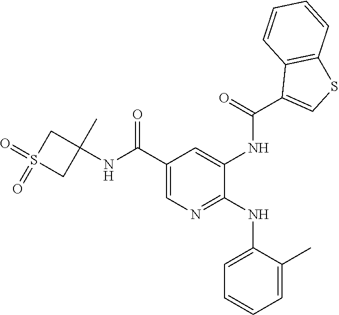 [SMILES: Cc1ccccc1Nc1ncc(C(=O)NC2(C)CS(=O)(=O)C2)cc1NC(=O)c1csc2ccccc12] | (400 MHz, dmso) 10.07 (s, 1H), 8.83 (s, 1H), 8.71 (s, 1H), 8.50 (dd, J = 7.3, 1.5 Hz, 1H), 8.46 (d, J = 2.2 Hz, 1H), 8.33 (s, 1H), 8.13 (d, J = 2.2 Hz, 1H), 8.09 (dd, J = 7.0, 1.1 Hz, 1H), 7.52- 7.43 (m, 2H), 7.42 (d, J = 7.4 Hz, 1H), 7.23 (dd, J = 7.8, 0.7 Hz, 1H), 7.18 (td, J = 7.4, 1.1 Hz, 1H), 7.07 (td, J = 7.4, 1.2 Hz, 1H), 4.53 (d, J = 14.9 Hz, 2H), 4.28 (d, J = 14.9 Hz, 2H), 2.17 (s, 3H), 1.71 (s, 3H). | 521.29 | B | B |
| I- 362 | 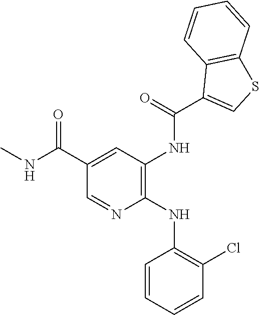 [SMILES: CNC(=O)c1cnc(Nc2ccccc2Cl)c(NC(=O)c2csc3ccccc23)c1] | | 437 | A | D |
| I- 363 | 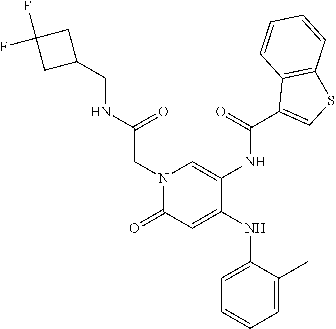 [SMILES: Cc1ccccc1Nc1cc(=O)n(CC(=O)NCC2CC(F)(F)C2)cc1NC(=O)c1csc2ccccc12] | | 537 | D | |
| I- 364 | 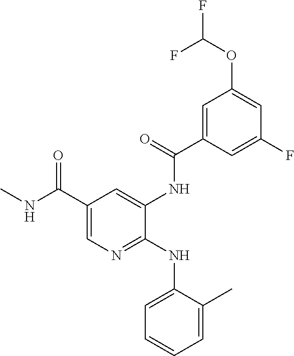 [SMILES: CNC(=O)c1cnc(Nc2ccccc2C)c(NC(=O)c2cc(F)cc(OC(F)F)c2)c1] | | 445.41 | D | |
| I- 365 | 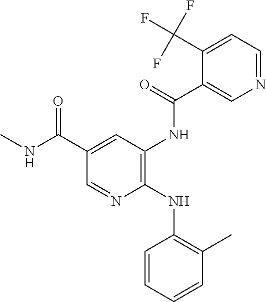 [SMILES: CNC(=O)c1cnc(Nc2ccccc2C)c(NC(=O)c2cnccc2C(F)(F)F)c1] | | | E | |
| I- 366 | 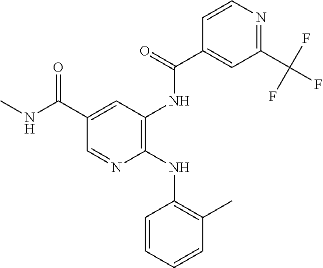 [SMILES: CNC(=O)c1cnc(Nc2ccccc2C)c(NC(=O)c2ccnc(C(F)(F)F)c2)c1] | | | E | |
| I- 367 | 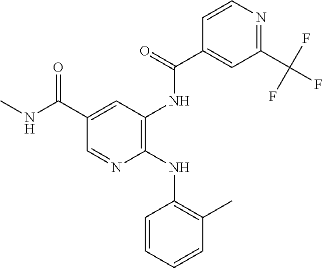 [SMILES: CNC(=O)c1cnc(Nc2ccccc2C)c(NC(=O)c2ccnc(C(F)(F)F)c2)c1] | | | E | |
| I- 368 | 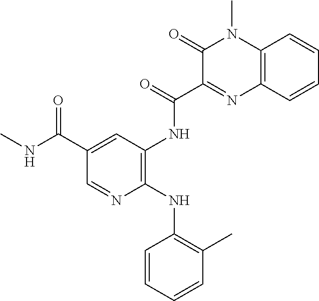 [SMILES: CNC(=O)c1cnc(Nc2ccccc2C)c(NC(=O)c2nc3ccccc3n(C)c2=O)c1] | | | E | |
| I- 369 | 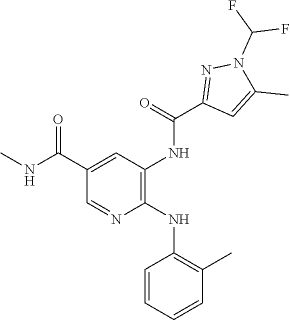 | | | E | |
| I- 370 | 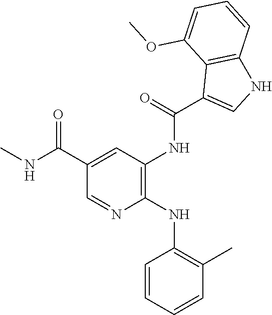 [SMILES: CNC(=O)c1cnc(Nc2ccccc2C)c(NC(=O)c2c[nH]c3cccc(OC)c23)c1] | | | E | |
| I- 371 | 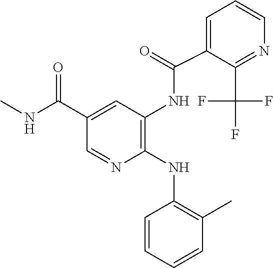 [SMILES: CNC(=O)c1cnc(Nc2ccccc2C)c(NC(=O)c2cccnc2C(F)(F)F)c1] | | | E | |
| I- 372 | 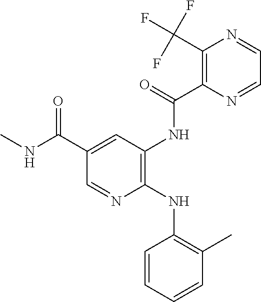 [SMILES: CNC(=O)c1cnc(Nc2ccccc2C)c(NC(=O)c2nccnc2C(F)(F)F)c1] | | | E | |
| I- 373 | 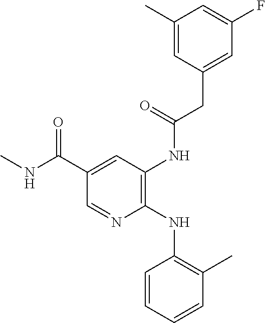 [SMILES: CNC(=O)c1cnc(Nc2ccccc2C)c(NC(=O)Cc2cc(C)cc(F)c2)c1] | | | E | |
| I- 374 | 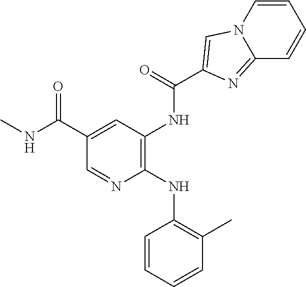 [SMILES: CNC(=O)c1cnc(Nc2ccccc2C)c(NC(=O)c2cn3ccccc3n2)c1] | | | E | |
| I- 375 | 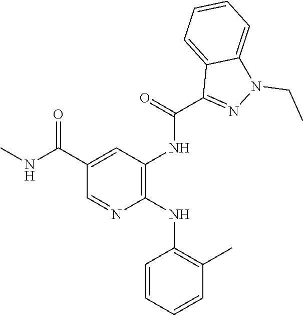 [SMILES: CCn1nc(C(=O)Nc2cc(C(=O)NC)cnc2Nc2ccccc2C)c2ccccc21] | | | E | |
| I- 376 | 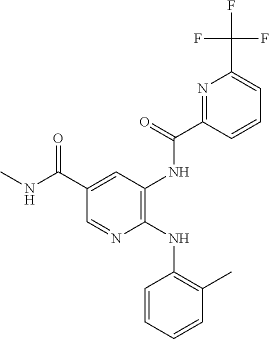 [SMILES: CNC(=O)c1cnc(Nc2ccccc2C)c(NC(=O)c2cccc(C(F)(F)F)n2)c1] | | | E | |
| I- 377 | 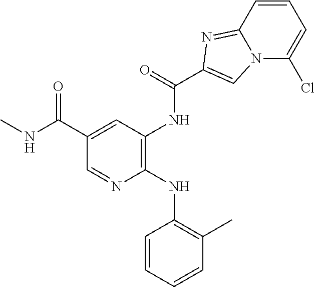 [SMILES: CNC(=O)c1cnc(Nc2ccccc2C)c(NC(=O)c2cn3c(Cl)cccc3n2)c1] | | | E | |
| I- 378 | 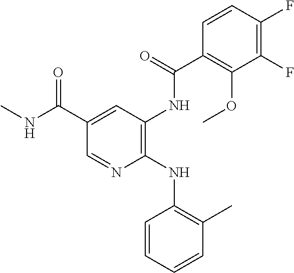 [SMILES: CNC(=O)c1cnc(Nc2ccccc2C)c(NC(=O)c2ccc(F)c(F)c2OC)c1] | | | E | |
| I- 379 | 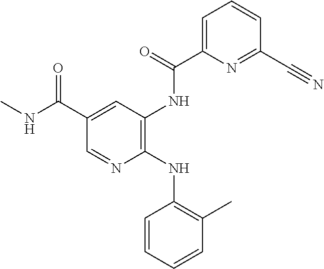 [SMILES: CNC(=O)c1cnc(Nc2ccccc2C)c(NC(=O)c2cccc(C#N)n2)c1] | | | E | |
| I- 380 | 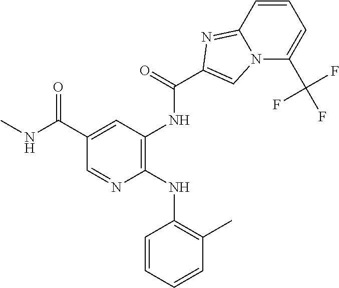 [SMILES: CNC(=O)c1cnc(Nc2ccccc2C)c(NC(=O)c2cn3c(C(F)(F)F)cccc3n2)c1] | | | E | |
| I- 381 | 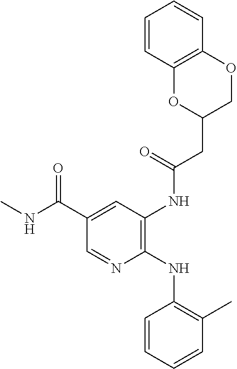 [SMILES: CNC(=O)c1cnc(Nc2ccccc2C)c(NC(=O)CC2COc3ccccc3O2)c1] | | | E | |
| I- 382 | 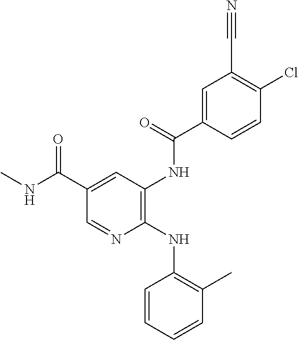 [SMILES: CNC(=O)c1cnc(Nc2ccccc2C)c(NC(=O)c2ccc(Cl)c(C#N)c2)c1] | | | E | |
| I- 383 | 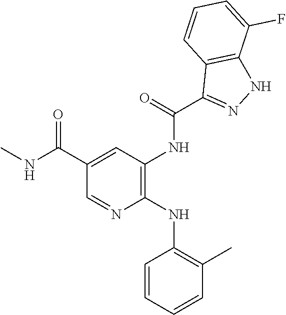 [SMILES: CNC(=O)c1cnc(Nc2ccccc2C)c(NC(=O)c2n[nH]c3c(F)cccc23)c1] | | | E | |
| I- 384 | 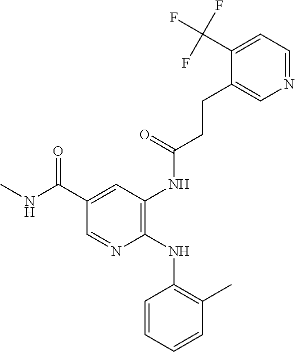 [SMILES: CNC(=O)c1cnc(Nc2ccccc2C)c(NC(=O)CCc2cnccc2C(F)(F)F)c1] | | | E | |
| I- 385 | 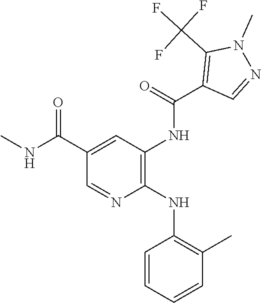 [SMILES: CNC(=O)c1cnc(Nc2ccccc2C)c(NC(=O)c2cnn(C)c2C(F)(F)F)c1] | | | E | |
| I- 386 | 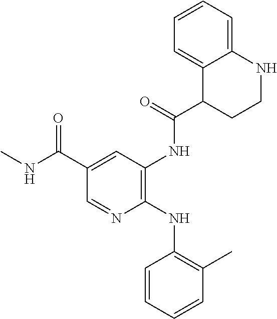 [SMILES: CNC(=O)c1cnc(Nc2ccccc2C)c(NC(=O)C2CCNc3ccccc32)c1] | | | E | |
| I- 387 | 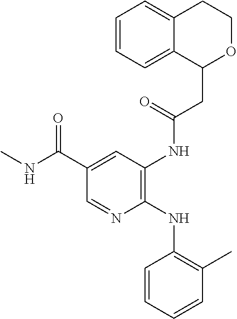 [SMILES: CNC(=O)c1cnc(Nc2ccccc2C)c(NC(=O)CC2OCCc3ccccc32)c1] | | | E | |
| I- 388 | 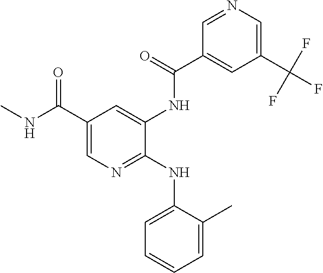 [SMILES: CNC(=O)c1cnc(Nc2ccccc2C)c(NC(=O)c2cncc(C(F)(F)F)c2)c1] | | 430.41 | E | |
| I- 389 | 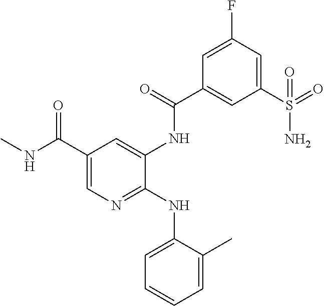 [SMILES: CNC(=O)c1cnc(Nc2ccccc2C)c(NC(=O)c2cc(F)cc(S(N)(=O)=O)c2)c1] | | 458.24 | E | |
| I- 390 | 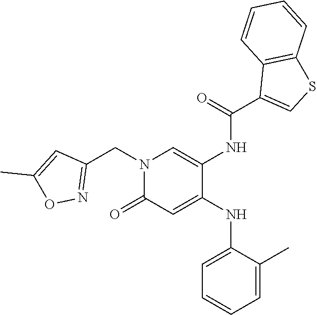 [SMILES: Cc1cc(Cn2cc(NC(=O)c3csc4ccccc34)c(Nc3ccccc3C)cc2=O)no1] | | 471 | B | D |
| I- 391 | 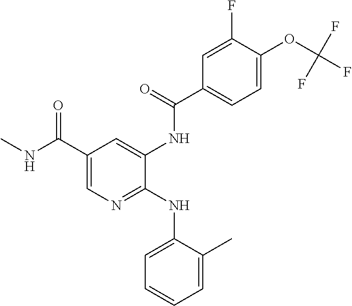 [SMILES: CNC(=O)c1cnc(Nc2ccccc2C)c(NC(=O)c2ccc(OC(F)(F)F)c(F)c2)c1] | | 463.41 | E | |
| I- 392 | 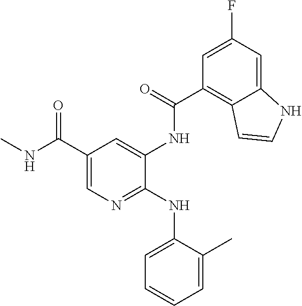 [SMILES: CNC(=O)c1cnc(Nc2ccccc2C)c(NC(=O)c2cc(F)cc3[nH]ccc23)c1] | | 418.15 | E | |
| I- 393 | 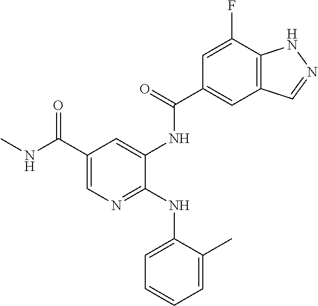 [SMILES: CNC(=O)c1cnc(Nc2ccccc2C)c(NC(=O)c2cc(F)c3[nH]ncc3c2)c1] | | 419.45 | E | |
| I- 394 | 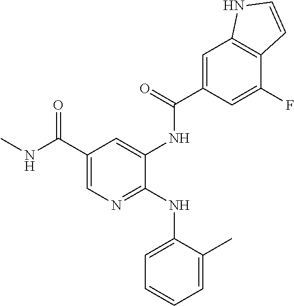 [SMILES: CNC(=O)c1cnc(Nc2ccccc2C)c(NC(=O)c2cc(F)c3cc[nH]c3c2)c1] | | 418.45 | E | |
| I- 395 | 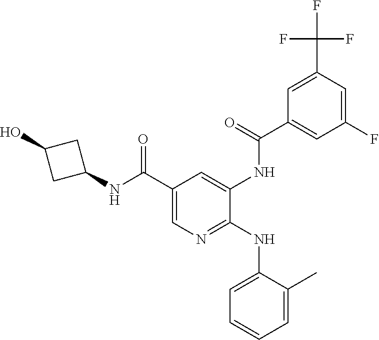 [SMILES: Cc1ccccc1Nc1ncc(C(=O)N[C@H]2C[C@@H](O)C2)cc1NC(=O)c1cc(F)cc(C(F)(F)F)c1] | | 503.3 | B | B |
| I- 396 | 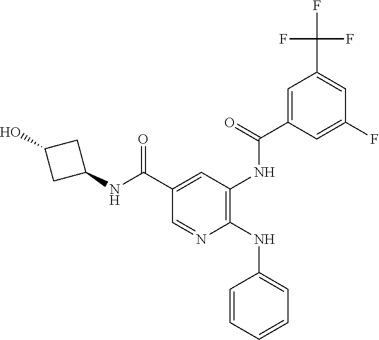 [SMILES: O=C(Nc1cc(C(=O)N[C@H]2C[C@H](O)C2)cnc1Nc1ccccc1)c1cc(F)cc(C(F)(F)F)c1] | | | B | B |
| I- 397 | 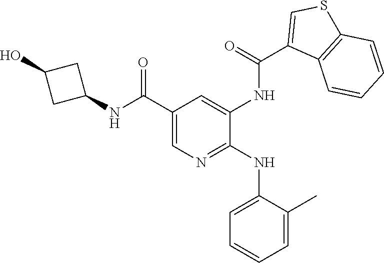 [SMILES: Cc1ccccc1Nc1ncc(C(=O)N[C@H]2C[C@@H](O)C2)cc1NC(=O)c1csc2ccccc12] | | 473.25 | A | C |
| I- 398 | 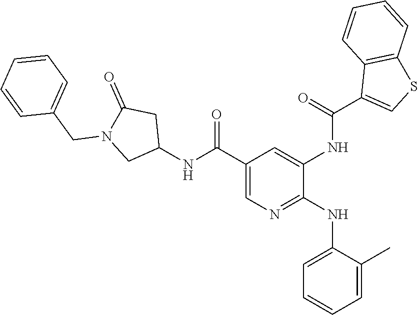 [SMILES: Cc1ccccc1Nc1ncc(C(=O)NC2CC(=O)N(Cc3ccccc3)C2)cc1NC(=O)c1csc2ccccc12] | | 576.43 | B | D |
| I- 399 | 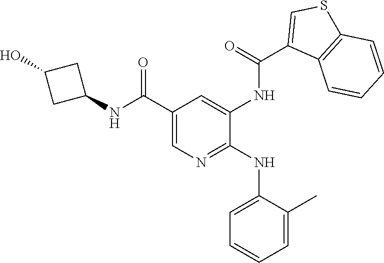 [SMILES: Cc1ccccc1Nc1ncc(C(=O)N[C@H]2C[C@H](O)C2)cc1NC(=O)c1csc2ccccc12] | | 473.35 | A | B |
| I- 400 | 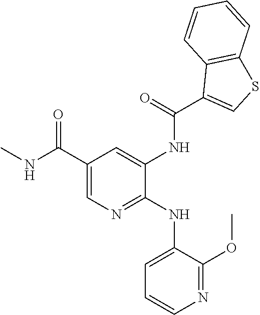 [SMILES: CNC(=O)c1cnc(Nc2cccnc2OC)c(NC(=O)c2csc3ccccc23)c1] | | | E | |
| I- 401 | 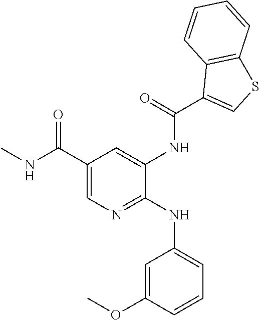 [SMILES: CNC(=O)c1cnc(Nc2cccc(OC)c2)c(NC(=O)c2csc3ccccc23)c1] | | | D | |
| I- 402 | 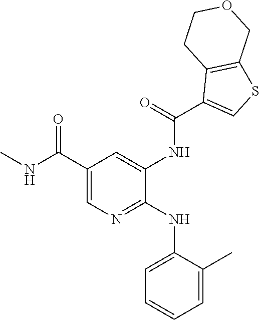 [SMILES: CNC(=O)c1cnc(Nc2ccccc2C)c(NC(=O)c2csc3c2CCOC3)c1] | | | D | D |
| I- 403 | 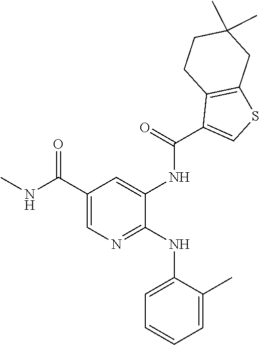 | | | E | |
| I- 404 | 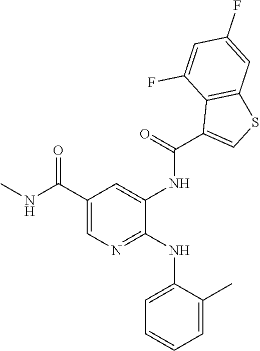 [SMILES: CNC(=O)c1cnc(Nc2ccccc2C)c(NC(=O)c2csc3cc(F)cc(F)c23)c1] | | | E | |
| I- 405 | 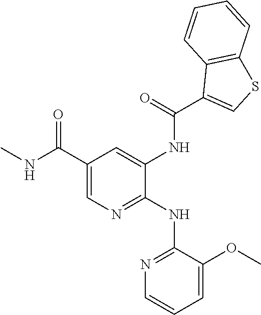 | | | E | E |
| I- 406 | 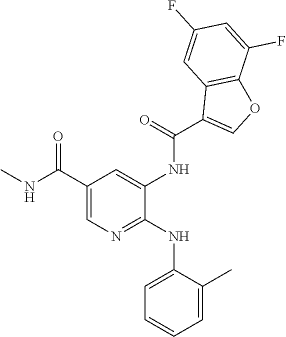 [SMILES: CNC(=O)c1cnc(Nc2ccccc2C)c(NC(=O)c2coc3c(F)cc(F)cc23)c1] | | | C | B |
| I- 407 | 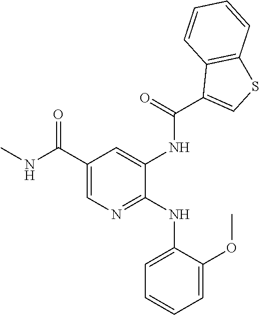 [SMILES: CNC(=O)c1cnc(Nc2ccccc2OC)c(NC(=O)c2csc3ccccc23)c1] | | | D | E |
| I- 408 | 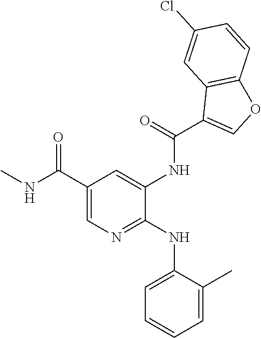 [SMILES: CNC(=O)c1cnc(Nc2ccccc2C)c(NC(=O)c2coc3ccc(Cl)cc23)c1] | | | C | B |
| I- 409 | 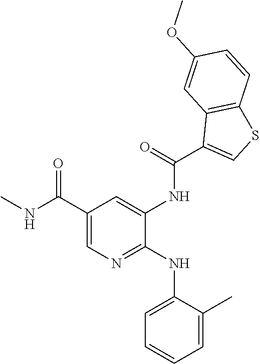 [SMILES: CNC(=O)c1cnc(Nc2ccccc2C)c(NC(=O)c2csc3ccc(OC)cc23)c1] | (400 MHz, dmso) 10.04 (s, 1H), 8.73 (s, 1H), 8.43 (d, J = 2.2 Hz, 1H), 8.32 (q, J = 4.3 Hz, 1H), 8.21 (s, 1H), 8.12 (d, J = 2.1 Hz, 1H), 8.04 (d, J = 2.6 Hz, 1H), 7.96 (d, J = 8.9 Hz, 1H), 7.46 (dd, J = 7.9, 0.9 Hz, 1H), 7.22 (d, J = 7.4 Hz, 1H), 7.18 (td, J = 7.7, 1.4 Hz, 1H), 7.10 (dd, J = 8.9, 2.6 Hz, 1H), 7.05 (td, J = 7.4, 1.2 Hz, 1H), 3.82 (s, 3H), 2.77 (d, J = 4.5 Hz, 3H), 2.18 (s, 3H) | 447.32 | D | D |
| I- 410 | 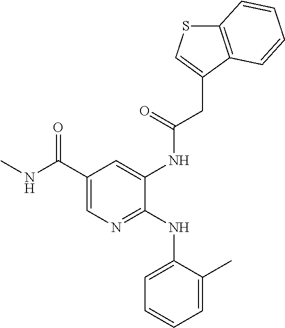 [SMILES: CNC(=O)c1cnc(Nc2ccccc2C)c(NC(=O)Cc2csc3ccccc23)c1] | (400 MHz, dmso) 9.91 (s, 1H), 8.37 (d, J = 2.2 Hz, 1H), 8.28 (q, J = 4.3 Hz, 1H), 8.02-7.98 (m, 2H), 7.96 (dd, J = 7.0, 1.7 Hz, 1H), 7.94 (s, 1H), 7.66 (s, 1H), 7.48 (d, J = 7.0 Hz, 1H), 7.41 (pd, J = 7.1, 1.4 Hz, 2H), 7.25-7.15 (m, 2H), 7.05 (td, J = 7.4, 1.2 Hz, 1H), 4.04 (s, 2H), 2.74 (d, J = 4.5 Hz, 3H), 2.07 (s, 3H). | 431.33 | E | |
| I- 411 | 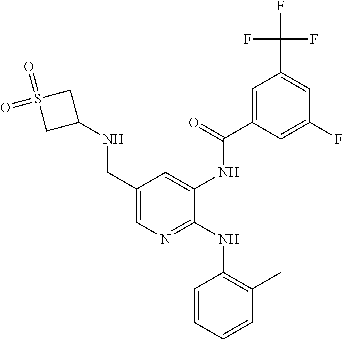 [SMILES: Cc1ccccc1Nc1ncc(CNC2CS(=O)(=O)C2)cc1NC(=O)c1cc(F)cc(C(F)(F)F)c1] | (400 MHz, dmso) 10.26 (s, 1H), 8.17 (s, 1H), 8.11 (d, J = 8.9 Hz, 1H), 7.94 (d, J = 8.9 Hz, 1H), 7.87 (d, J = 2.1 Hz, 1H), 7.67 (s, 1H), 7.59 (d, J = 2.1 Hz, 1H), 7.38 (d, J = 7.5 Hz, 1H), 7.14 (d, J = 7.5 Hz, 1H), 7.09 (t, J = 7.5 Hz, 1H), 6.94 (t, J = 7.5 Hz, 1H), 4.35-4.15 (m, 2H), 3.90-3.84 (m, 2H), 3.52 (s, 2H), 3.50-3.45 (m, 1H), 2.12 (s, 3H). | 523.3 | D | D |
| I- 412 | 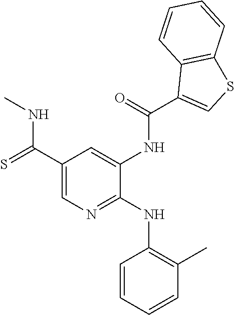 [SMILES: CNC(=S)c1cnc(Nc2ccccc2C)c(NC(=O)c2csc3ccccc23)c1] | (400 MHz, cd3od) 8.52 (br s, 1H), 8.51-8.48 (m, 1H), 8.45 (s, 1H), 8.44 (d, J = 2.3 Hz, 1H), 8.20 (d, J = 2.3 Hz, 1H), 7.97-7.93 (m, 1H), 7.49-7.41 (m, 3H), 7.22 (d, J = 7.5 Hz, 1H), 7.17 (td, J = 7.6, 1.3 Hz, 1H), 7.07 (td, J = 7.4, 1.3 Hz, 1H), 3.23 (s, 3H), 2.24 (s, 3H). | 433.27 | B | D |
| I- 413 | 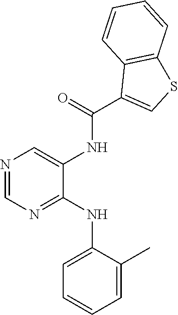 [SMILES: Cc1ccccc1Nc1ncncc1NC(=O)c1csc2ccccc12] | (400 MHz, dmso) 10.01 (s, 1H), 8.69 (s, 1H), 8.67 (s, 1H), 8.52-8.47 (m, 1H), 8.35 (s, 1H), 8.34 (s, 1H), 8.16 (s, 1H), 8.11-8.06 (m, 1H), 7.53-7.42 (m, 2H), 7.31 (dd, J = 7.8, 1.3 Hz, 1H), 7.26 (dd, J = 6.8, 0.6 Hz, 1H), 7.21 (td, J = 7.4, 1.4 Hz, 1H), 7.14 (td, J = 7.4, 1.4 Hz, 1H), 2.19 (s, 3H). | 361.27 | D | D |
| I- 414 | 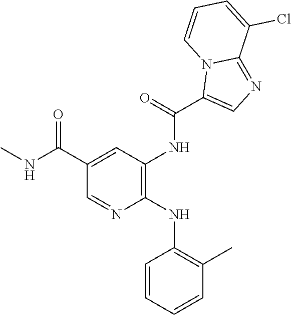 [SMILES: CNC(=O)c1cnc(Nc2ccccc2C)c(NC(=O)c2cnc3c(Cl)cccn23)c1] | | | E | |
| I- 415 | 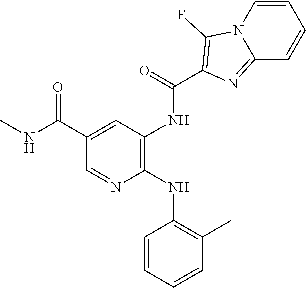 [SMILES: CNC(=O)c1cnc(Nc2ccccc2C)c(NC(=O)c2nc3ccccn3c2F)c1] | | | E | |
| I- 416 | 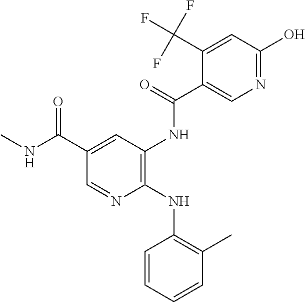 [SMILES: CNC(=O)c1cnc(Nc2ccccc2C)c(NC(=O)c2cnc(O)cc2C(F)(F)F)c1] | | | E | |
| I- 417 | 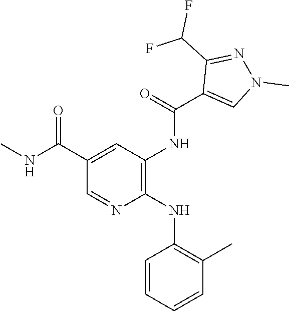 | | | E | |
| I- 418 | 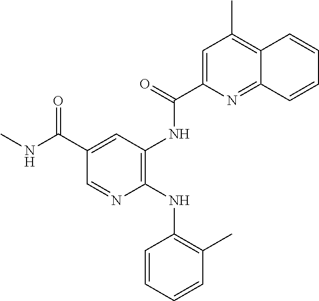 [SMILES: CNC(=O)c1cnc(Nc2ccccc2C)c(NC(=O)c2cc(C)c3ccccc3n2)c1] | | | E | |
| I- 419 | 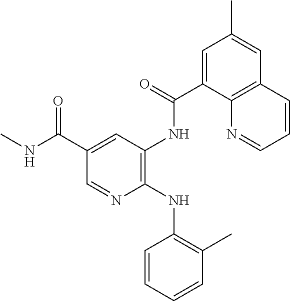 [SMILES: CNC(=O)c1cnc(Nc2ccccc2C)c(NC(=O)c2cc(C)cc3cccnc23)c1] | | | D | |
| I- 420 | 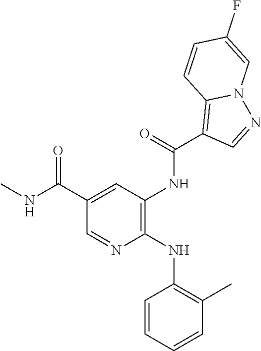 [SMILES: CNC(=O)c1cnc(Nc2ccccc2C)c(NC(=O)c2cnn3cc(F)ccc23)c1] | | | E | |
| I- 421 | 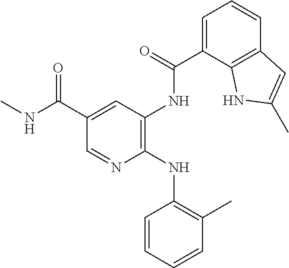 | | | D | |
| I- 422 | 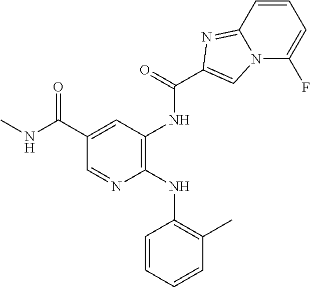 [SMILES: CNC(=O)c1cnc(Nc2ccccc2C)c(NC(=O)c2cn3c(F)cccc3n2)c1] | | | E | |
| I- 423 | 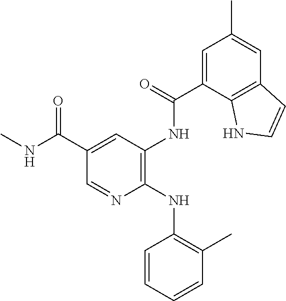 [SMILES: CNC(=O)c1cnc(Nc2ccccc2C)c(NC(=O)c2cc(C)cc3cc[nH]c23)c1] | | | D | |
| I- 424 | 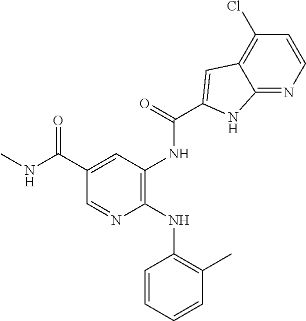 [SMILES: CNC(=O)c1cnc(Nc2ccccc2C)c(NC(=O)c2cc3c(Cl)ccnc3[nH]2)c1] | | | E | E |
| I- 425 | 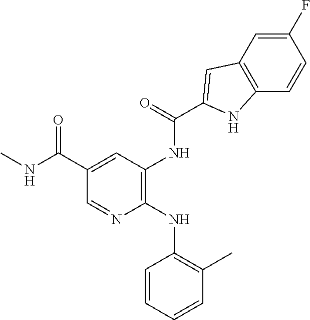 [SMILES: CNC(=O)c1cnc(Nc2ccccc2C)c(NC(=O)c2cc3cc(F)ccc3[nH]2)c1] | | | E | |
| I- 426 | 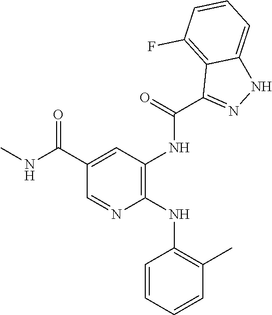 [SMILES: CNC(=O)c1cnc(Nc2ccccc2C)c(NC(=O)c2n[nH]c3cccc(F)c23)c1] | | | D | |
| I- 427 | 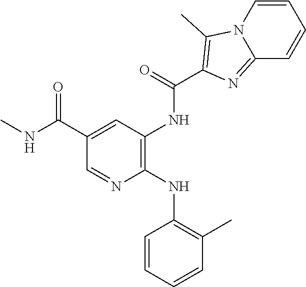 [SMILES: CNC(=O)c1cnc(Nc2ccccc2C)c(NC(=O)c2nc3ccccn3c2C)c1] | | | E | |
| I- 428 | 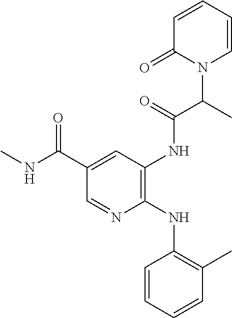 [SMILES: CNC(=O)c1cnc(Nc2ccccc2C)c(NC(=O)C(C)n2ccccc2=O)c1] | | | E | |
| I- 429 | 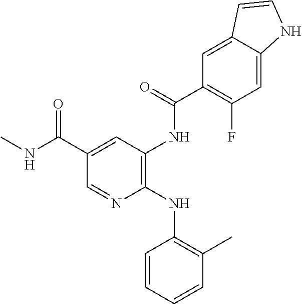 [SMILES: CNC(=O)c1cnc(Nc2ccccc2C)c(NC(=O)c2cc3cc[nH]c3cc2F)c1] | | | E | |
| I- 430 | 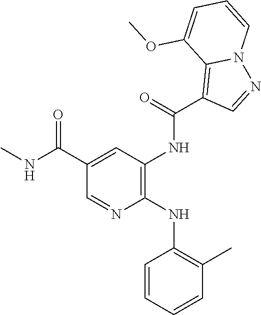 [SMILES: CNC(=O)c1cnc(Nc2ccccc2C)c(NC(=O)c2cnn3cccc(OC)c23)c1] | | | E | |
| I- 431 | 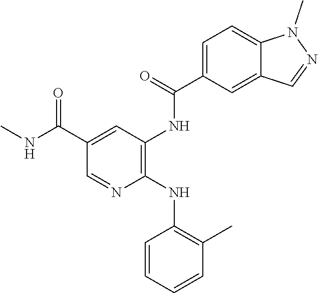 [SMILES: CNC(=O)c1cnc(Nc2ccccc2C)c(NC(=O)c2ccc3c(cnn3C)c2)c1] | | | E | |
| I- 432 | 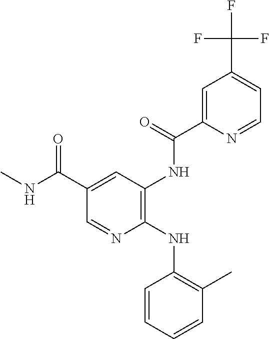 [SMILES: CNC(=O)c1cnc(Nc2ccccc2C)c(NC(=O)c2cc(C(F)(F)F)ccn2)c1] | | | E | |
| I- 433 | 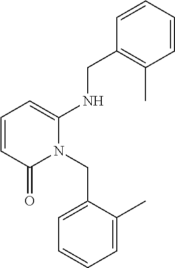 [SMILES: Cc1ccccc1CNc1cccc(=O)n1Cc1ccccc1C] | | 319.33 | E | |
| I- 434 | 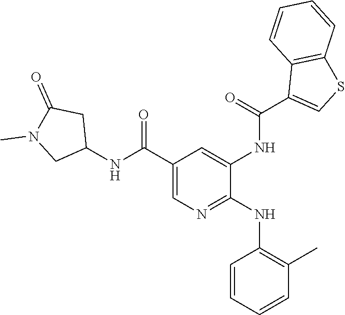 [SMILES: Cc1ccccc1Nc1ncc(C(=O)NC2CC(=O)N(C)C2)cc1NC(=O)c1csc2ccccc12] | | 500.3 | B | C |
| I- 435 | 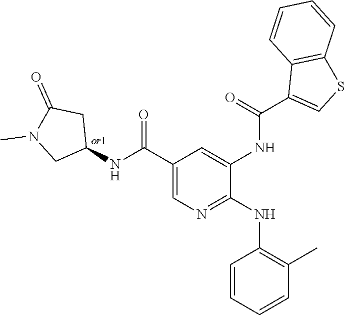 [SMILES: Cc1ccccc1Nc1ncc(C(=O)N[C@@H]2CC(=O)N(C)C2)cc1NC(=O)c1csc2ccccc12] | | 500.3 | A | C |
| I- 436 | 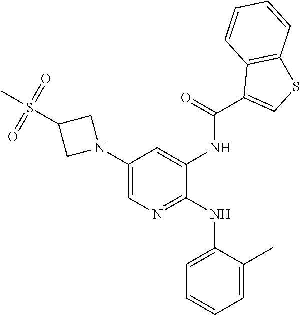 [SMILES: Cc1ccccc1Nc1ncc(N2CC(S(C)(=O)=O)C2)cc1NC(=O)c1csc2ccccc12] | (400 MHz, dmso) 10.15 (s, 1H), 8.59 (s, 1H), 8.49-8.39 (m, 1H), 8.12-8.08 (m, 1H), 7.55-7.41 (m, 3H), 7.39 (dd, J = 4.7, 1.9 Hz, 1H), 7.21 (s, 1H), 7.17 (d, J = 2.8 Hz, 1H), 7.10 (d, J = 7.3 Hz, 1H), 7.05 (t, J = 7.7 Hz, 1H), 6.79 (td, J = 7.4, 1.2 Hz, 1H), 4.54-4.41 (m, 1H), 4.25 (t, J = 8.3 Hz, 1H), 4.19-4.13 (m, 2H), 4.07 (dd, J = 8.4, 5.5 Hz, 1H), 3.06 (s, 3H), 2.15 (s, 3H). | 493.3 | C | D |
| I- 437 | 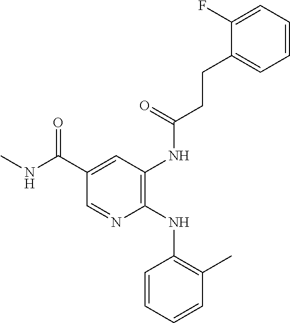 [SMILES: CNC(=O)c1cnc(Nc2ccccc2C)c(NC(=O)CCc2ccccc2F)c1] | | | E | |
| I- 438 | 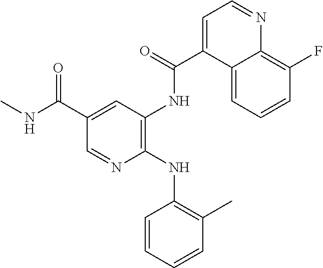 [SMILES: CNC(=O)c1cnc(Nc2ccccc2C)c(NC(=O)c2ccnc3c(F)cccc23)c1] | | | E | |
| I- 439 | 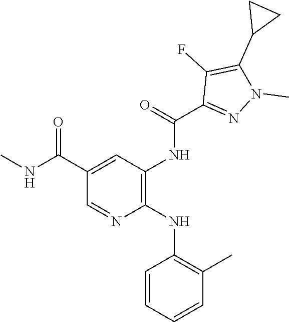 | | | E | |
| I- 440 | 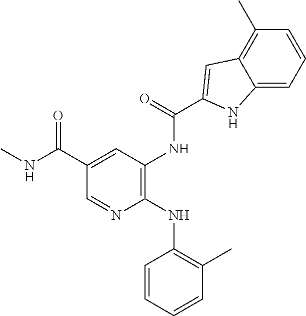 [SMILES: CNC(=O)c1cnc(Nc2ccccc2C)c(NC(=O)c2cc3c(C)cccc3[nH]2)c1] | | | E | |
| I- 441 | 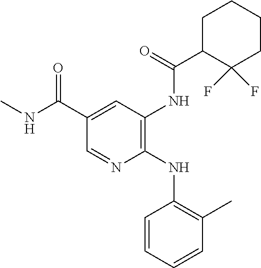 [SMILES: CNC(=O)c1cnc(Nc2ccccc2C)c(NC(=O)C2CCCCC2(F)F)c1] | | | E | |
| I- 442 | 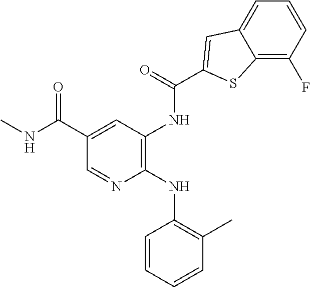 [SMILES: CNC(=O)c1cnc(Nc2ccccc2C)c(NC(=O)c2cc3cccc(F)c3s2)c1] | | | E | |
| I- 443 | 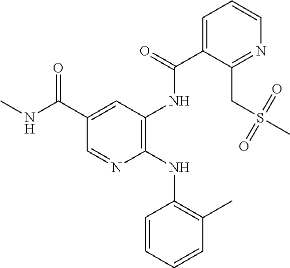 | | | E | |
| I- 444 | 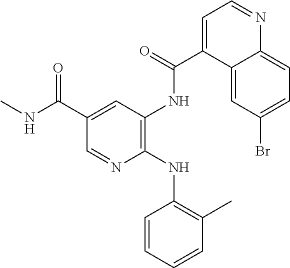 [SMILES: CNC(=O)c1cnc(Nc2ccccc2C)c(NC(=O)c2ccnc3ccc(Br)cc23)c1] | | | E | |
| I- 445 | 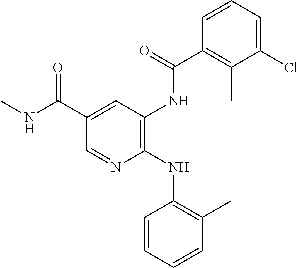 [SMILES: CNC(=O)c1cnc(Nc2ccccc2C)c(NC(=O)c2cccc(Cl)c2C)c1] | | | E | |
| I- 446 | 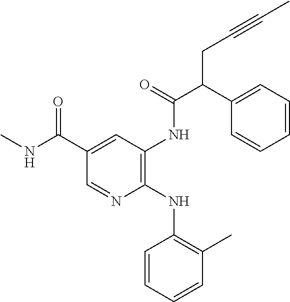 [SMILES: CC#CCC(C(=O)Nc1cc(C(=O)NC)cnc1Nc1ccccc1C)c1ccccc1] | | | E | |
| I- 447 | 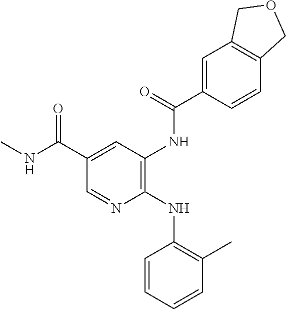 [SMILES: CNC(=O)c1cnc(Nc2ccccc2C)c(NC(=O)c2ccc3c(c2)COC3)c1] | | | E | |
| I- 448 | 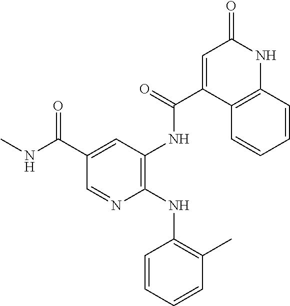 [SMILES: CNC(=O)c1cnc(Nc2ccccc2C)c(NC(=O)c2cc(=O)[nH]c3ccccc23)c1] | | | E | |
| I- 449 | 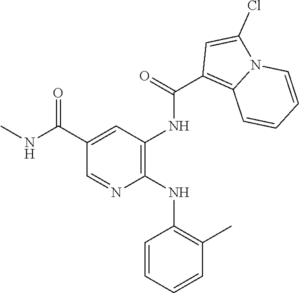 [SMILES: CNC(=O)c1cnc(Nc2ccccc2C)c(NC(=O)c2cc(Cl)n3ccccc23)c1] | | 434 | B | |
| I- 450 | 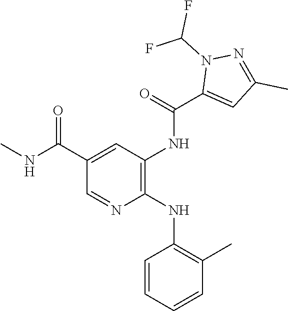 | | | E | |
| I- 451 | 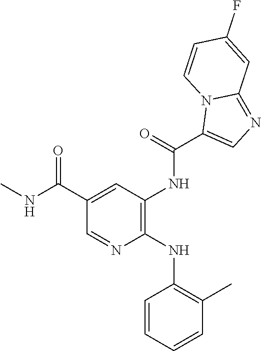 [SMILES: CNC(=O)c1cnc(Nc2ccccc2C)c(NC(=O)c2cnc3cc(F)ccn23)c1] | | | E | |
| I- 452 | 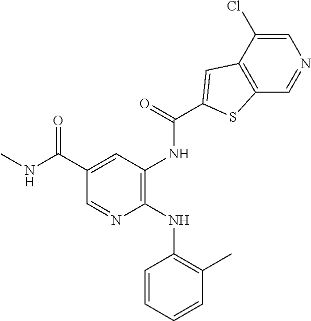 [SMILES: CNC(=O)c1cnc(Nc2ccccc2C)c(NC(=O)c2cc3c(Cl)cncc3s2)c1] | | | E | |
| I- 453 | 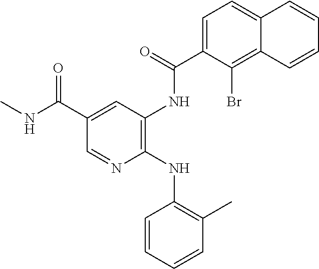 [SMILES: CNC(=O)c1cnc(Nc2ccccc2C)c(NC(=O)c2ccc3ccccc3c2Br)c1] | | | E | |
| I- 454 | 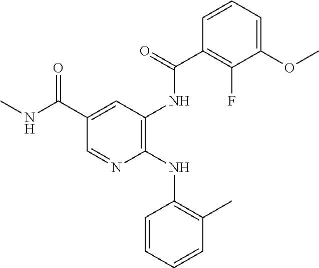 | | | E | |
| I- 455 | 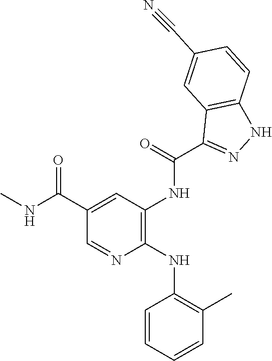 [SMILES: CNC(=O)c1cnc(Nc2ccccc2C)c(NC(=O)c2n[nH]c3ccc(C#N)cc23)c1] | | | E | |
| I- 456 | 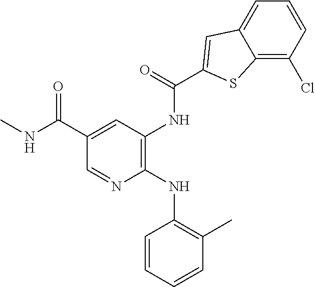 [SMILES: CNC(=O)c1cnc(Nc2ccccc2C)c(NC(=O)c2cc3cccc(Cl)c3s2)c1] | | | D | |
| I- 457 | 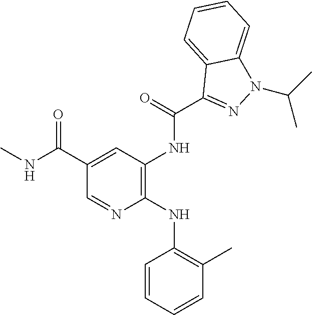 [SMILES: CNC(=O)c1cnc(Nc2ccccc2C)c(NC(=O)c2nn(C(C)C)c3ccccc23)c1] | | | E | |
| I- 458 | 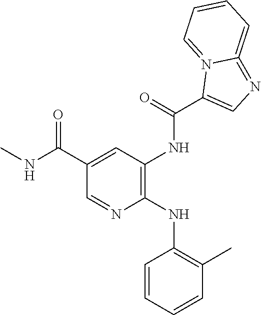 | | | E | |
| I- 459 | 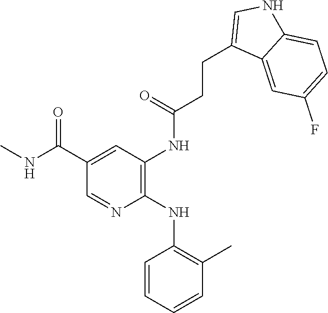 [SMILES: CNC(=O)c1cnc(Nc2ccccc2C)c(NC(=O)CCc2c[nH]c3ccc(F)cc23)c1] | | | E | |
| I- 460 | 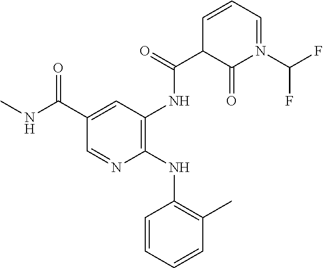 | | | E | |
| I- 461 | 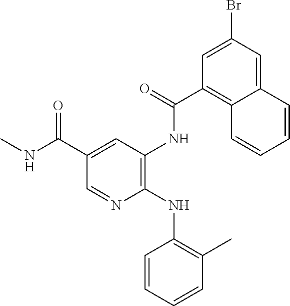 [SMILES: CNC(=O)c1cnc(Nc2ccccc2C)c(NC(=O)c2cc(Br)cc3ccccc23)c1] | | | E | |
| I- 462 | 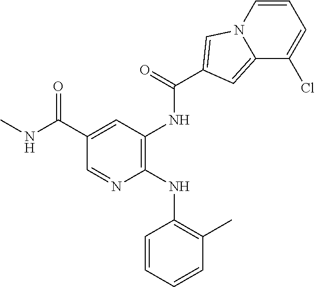 [SMILES: CNC(=O)c1cnc(Nc2ccccc2C)c(NC(=O)c2cc3c(Cl)cccn3c2)c1] | | | E | |
| I- 463 | 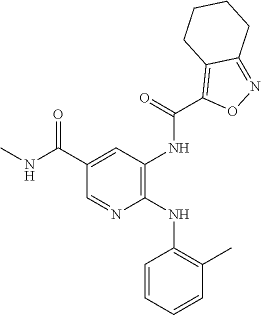 [SMILES: CNC(=O)c1cnc(Nc2ccccc2C)c(NC(=O)c2onc3c2CCCC3)c1] | | | E | |
| I- 464 | 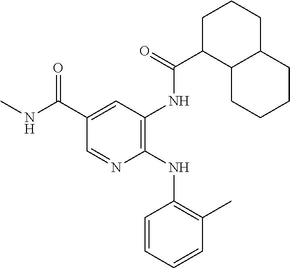 [SMILES: CNC(=O)c1cnc(Nc2ccccc2C)c(NC(=O)C2CCCC3CCCCC32)c1] | | | E | |
| I- 465 | 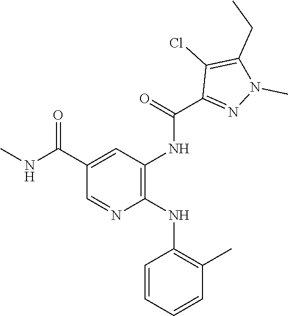 | | | E | |
| I- 466 | 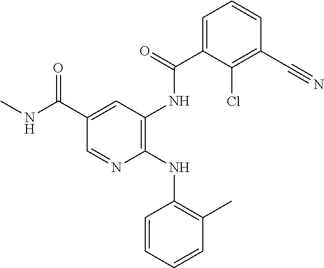 [SMILES: CNC(=O)c1cnc(Nc2ccccc2C)c(NC(=O)c2cccc(C#N)c2Cl)c1] | | | E | |
| I- 467 | 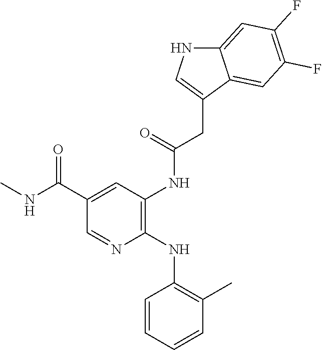 [SMILES: CNC(=O)c1cnc(Nc2ccccc2C)c(NC(=O)Cc2c[nH]c3cc(F)c(F)cc23)c1] | | | E | |
| I- 468 | 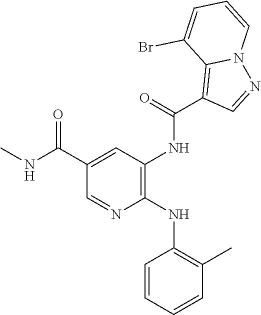 [SMILES: CNC(=O)c1cnc(Nc2ccccc2C)c(NC(=O)c2cnn3cccc(Br)c23)c1] | | | D | D |
| I- 469 | 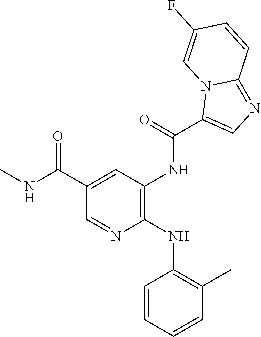 [SMILES: CNC(=O)c1cnc(Nc2ccccc2C)c(NC(=O)c2cnc3ccc(F)cn23)c1] | | | E | |
| I- 470 | 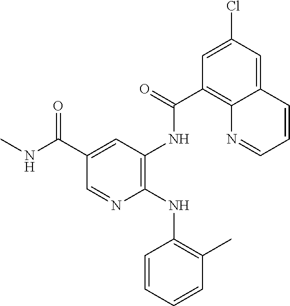 [SMILES: CNC(=O)c1cnc(Nc2ccccc2C)c(NC(=O)c2cc(Cl)cc3cccnc23)c1] | | | E | |
| I- 471 | 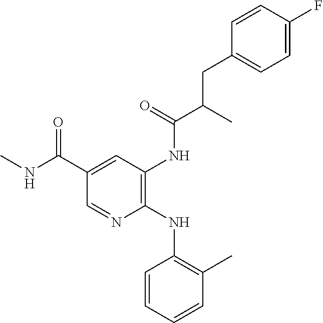 [SMILES: CNC(=O)c1cnc(Nc2ccccc2C)c(NC(=O)C(C)Cc2ccc(F)cc2)c1] | | | E | |
| I- 472 | 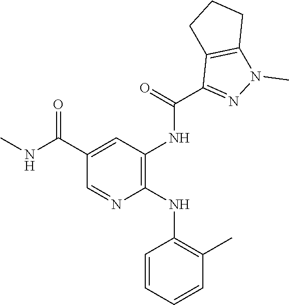 | | | E | |
| I- 473 | 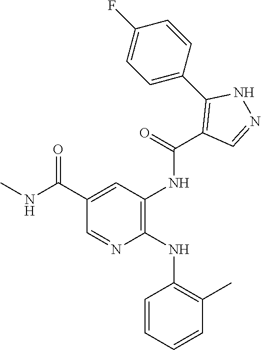 [SMILES: CNC(=O)c1cnc(Nc2ccccc2C)c(NC(=O)c2cn[nH]c2-c2ccc(F)cc2)c1] | | | E | |
| I- 474 | 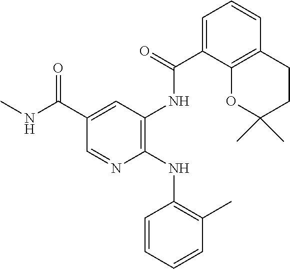 [SMILES: CNC(=O)c1cnc(Nc2ccccc2C)c(NC(=O)c2cccc3c2OC(C)(C)CC3)c1] | | | E | |
| I- 475 | 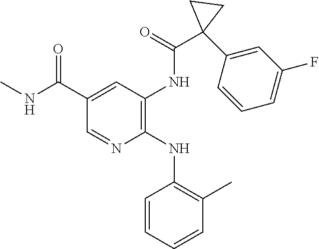 [SMILES: CNC(=O)c1cnc(Nc2ccccc2C)c(NC(=O)C2(c3cccc(F)c3)CC2)c1] | | | D | |
| I- 476 | 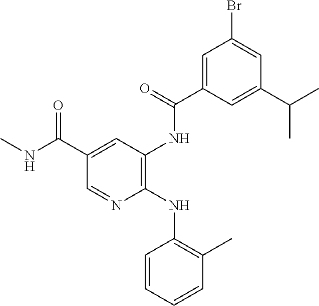 [SMILES: CNC(=O)c1cnc(Nc2ccccc2C)c(NC(=O)c2cc(Br)cc(C(C)C)c2)c1] | | | E | |
| I- 477 | 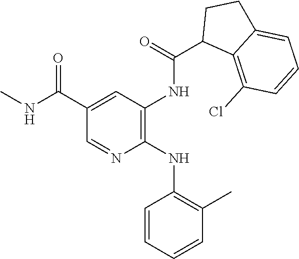 [SMILES: CNC(=O)c1cnc(Nc2ccccc2C)c(NC(=O)C2CCc3cccc(Cl)c32)c1] | | | E | |
| I- 478 | 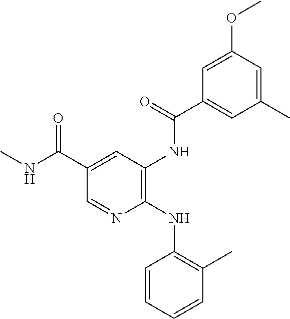 | | | E | |
| I- 479 | 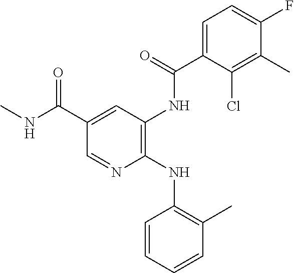 [SMILES: CNC(=O)c1cnc(Nc2ccccc2C)c(NC(=O)c2ccc(F)c(C)c2Cl)c1] | | | E | |
| I- 480 | 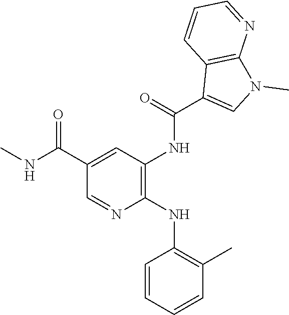 | | | E | |
| I- 481 | 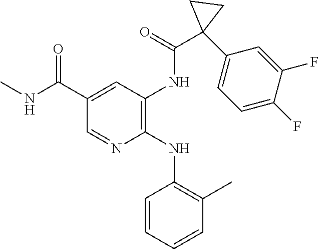 [SMILES: CNC(=O)c1cnc(Nc2ccccc2C)c(NC(=O)C2(c3ccc(F)c(F)c3)CC2)c1] | | | E | |
| I- 482 | 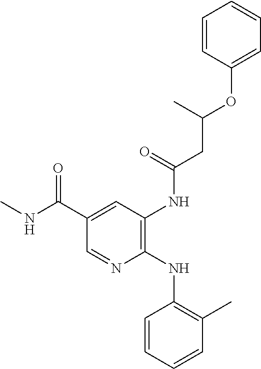 [SMILES: CNC(=O)c1cnc(Nc2ccccc2C)c(NC(=O)CC(C)Oc2ccccc2)c1] | | | E | |
| I- 483 | 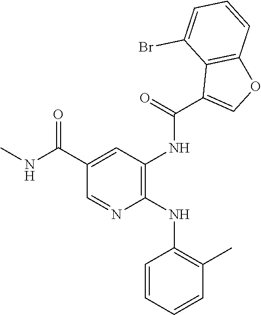 [SMILES: CNC(=O)c1cnc(Nc2ccccc2C)c(NC(=O)c2coc3cccc(Br)c23)c1] | | | E | |
| I- 484 | 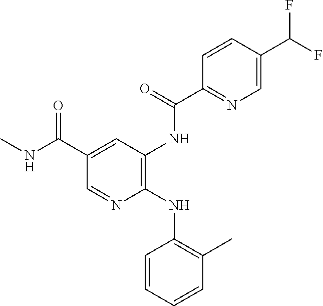 [SMILES: CNC(=O)c1cnc(Nc2ccccc2C)c(NC(=O)c2ccc(C(F)F)cn2)c1] | | | E | |
| I- 485 | 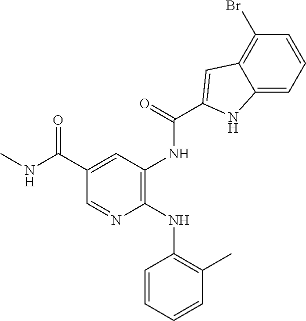 [SMILES: CNC(=O)c1cnc(Nc2ccccc2C)c(NC(=O)c2cc3c(Br)cccc3[nH]2)c1] | | | E | |
| I- 486 | 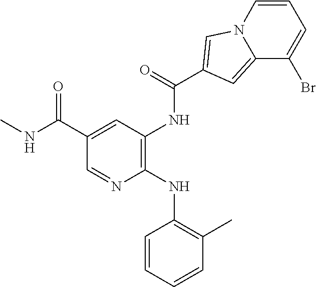 [SMILES: CNC(=O)c1cnc(Nc2ccccc2C)c(NC(=O)c2cc3c(Br)cccn3c2)c1] | | | B | D |
| I- 487 | 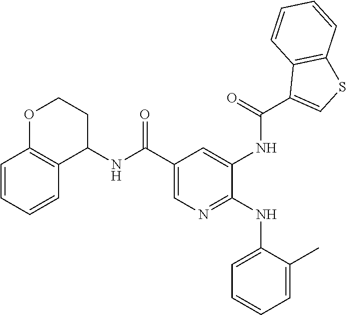 [SMILES: Cc1ccccc1Nc1ncc(C(=O)NC2CCOc3ccccc32)cc1NC(=O)c1csc2ccccc12] | | | E | |
| I- 488 | 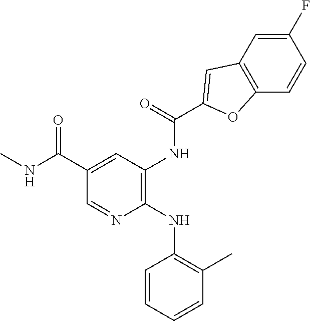 [SMILES: CNC(=O)c1cnc(Nc2ccccc2C)c(NC(=O)c2cc3cc(F)ccc3o2)c1] | | | E | |
| I- 489 | 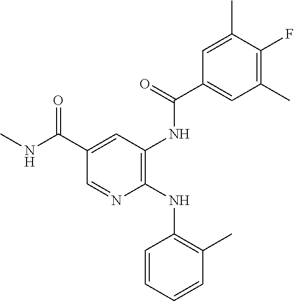 [SMILES: CNC(=O)c1cnc(Nc2ccccc2C)c(NC(=O)c2cc(C)c(F)c(C)c2)c1] | | | D | |
| I- 490 | 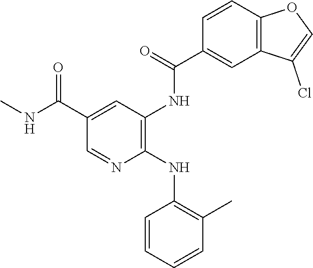 [SMILES: CNC(=O)c1cnc(Nc2ccccc2C)c(NC(=O)c2ccc3occ(Cl)c3c2)c1] | | | E | |
| I- 491 | 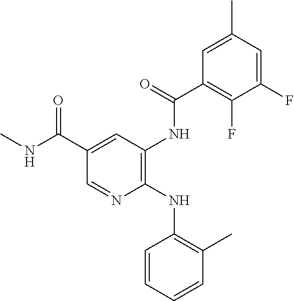 [SMILES: CNC(=O)c1cnc(Nc2ccccc2C)c(NC(=O)c2cc(C)cc(F)c2F)c1] | | | E | |
| I- 492 | 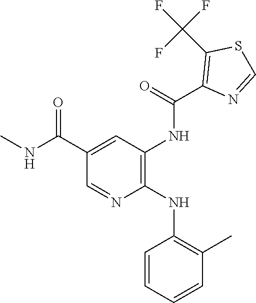 [SMILES: CNC(=O)c1cnc(Nc2ccccc2C)c(NC(=O)c2ncsc2C(F)(F)F)c1] | | | E | |
| I- 493 | 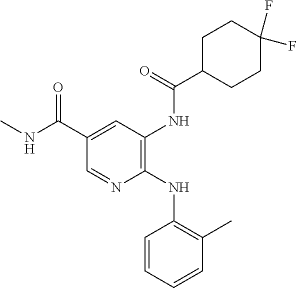 [SMILES: CNC(=O)c1cnc(Nc2ccccc2C)c(NC(=O)C2CCC(F)(F)CC2)c1] | | | E | |
| I- 494 | 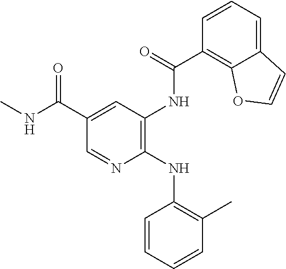 [SMILES: CNC(=O)c1cnc(Nc2ccccc2C)c(NC(=O)c2cccc3ccoc23)c1] | | | E | |
| I- 495 | 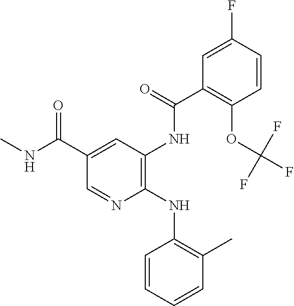 [SMILES: CNC(=O)c1cnc(Nc2ccccc2C)c(NC(=O)c2cc(F)ccc2OC(F)(F)F)c1] | | | E | |
| I- 496 | 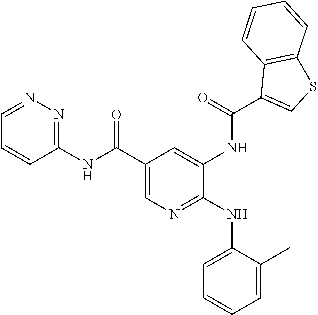 [SMILES: Cc1ccccc1Nc1ncc(C(=O)Nc2cccnn2)cc1NC(=O)c1csc2ccccc12] | | | C | D |
| I- 497 | 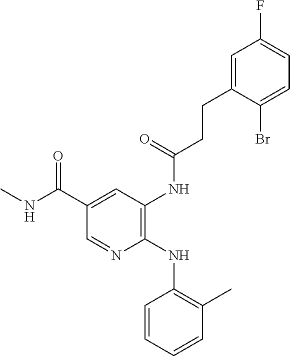 [SMILES: CNC(=O)c1cnc(Nc2ccccc2C)c(NC(=O)CCc2cc(F)ccc2Br)c1] | | | E | |
| I- 499 | 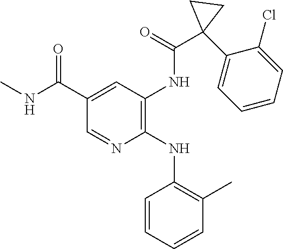 [SMILES: CNC(=O)c1cnc(Nc2ccccc2C)c(NC(=O)C2(c3ccccc3Cl)CC2)c1] | | | A | D |
| I- 500 | 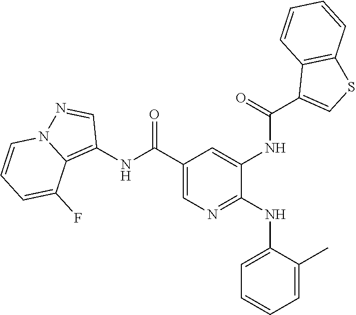 [SMILES: Cc1ccccc1Nc1ncc(C(=O)Nc2cnn3cccc(F)c23)cc1NC(=O)c1csc2ccccc12] | | | E | |
| I- 501 | 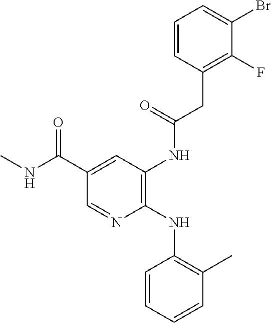 [SMILES: CNC(=O)c1cnc(Nc2ccccc2C)c(NC(=O)Cc2cccc(Br)c2F)c1] | | | E | |
| I- 502 | 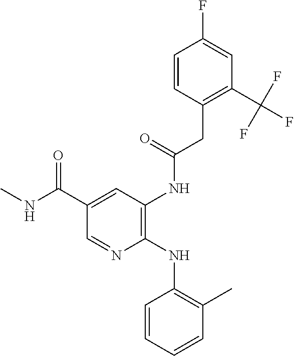 [SMILES: CNC(=O)c1cnc(Nc2ccccc2C)c(NC(=O)Cc2ccc(F)cc2C(F)(F)F)c1] | | | E | |
| I- 503 | 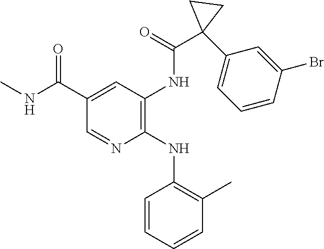 [SMILES: CNC(=O)c1cnc(Nc2ccccc2C)c(NC(=O)C2(c3cccc(Br)c3)CC2)c1] | | | E | |
| I- 504 | 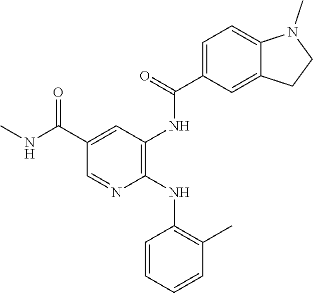 [SMILES: CNC(=O)c1cnc(Nc2ccccc2C)c(NC(=O)c2ccc3c(c2)CCN3C)c1] | | | E | |
| I- 505 | 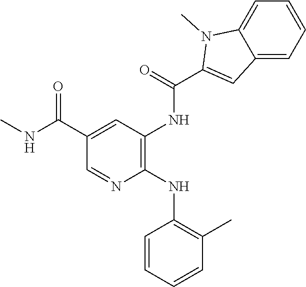 [SMILES: CNC(=O)c1cnc(Nc2ccccc2C)c(NC(=O)c2cc3ccccc3n2C)c1] | | | E | |
| I- 506 | 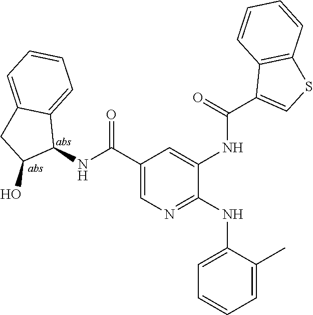 [SMILES: Cc1ccccc1Nc1ncc(C(=O)N[C@@H]2c3ccccc3C[C@@H]2O)cc1NC(=O)c1csc2ccccc12] | | | B | C |
| I- 507 | 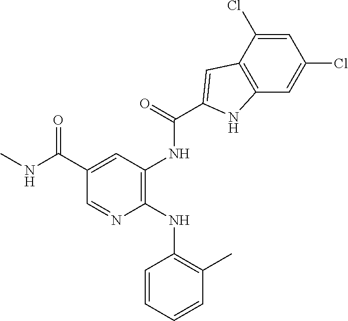 [SMILES: CNC(=O)c1cnc(Nc2ccccc2C)c(NC(=O)c2cc3c(Cl)cc(Cl)cc3[nH]2)c1] | | | D | |
| I- 508 | 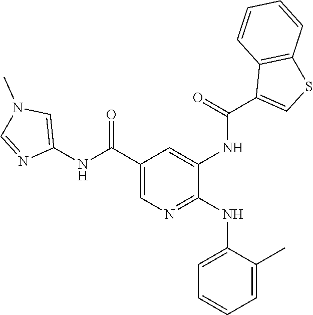 [SMILES: Cc1ccccc1Nc1ncc(C(=O)Nc2cn(C)cn2)cc1NC(=O)c1csc2ccccc12] | | | B | D |
| I- 509 | 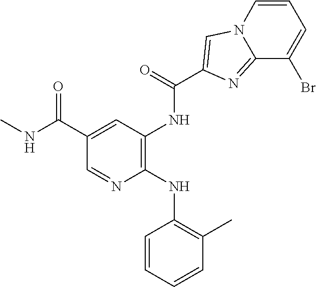 [SMILES: CNC(=O)c1cnc(Nc2ccccc2C)c(NC(=O)c2cn3cccc(Br)c3n2)c1] | | | E | |
| I- 510 | 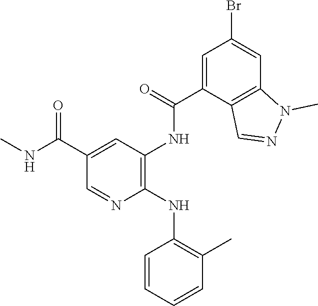 | | | B | D |
| I- 511 | 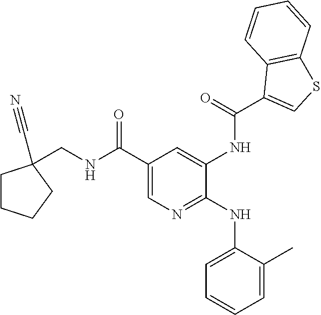 [SMILES: Cc1ccccc1Nc1ncc(C(=O)NCC2(C#N)CCCC2)cc1NC(=O)c1csc2ccccc12] | | | E | |
| I- 512 | 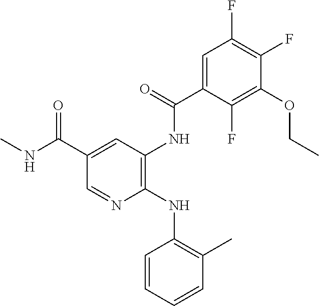 [SMILES: CCOc1c(F)c(F)cc(C(=O)Nc2cc(C(=O)NC)cnc2Nc2ccccc2C)c1F] | | | E | |
| I- 513 | 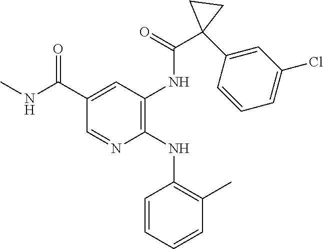 [SMILES: CNC(=O)c1cnc(Nc2ccccc2C)c(NC(=O)C2(c3cccc(Cl)c3)CC2)c1] | | | E | |
| I- 514 | 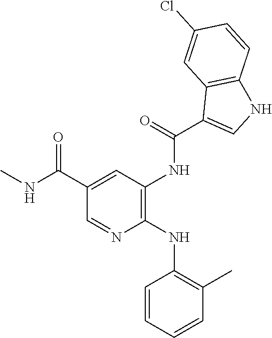 [SMILES: CNC(=O)c1cnc(Nc2ccccc2C)c(NC(=O)c2c[nH]c3ccc(Cl)cc23)c1] | | | E | |
| I- 515 | 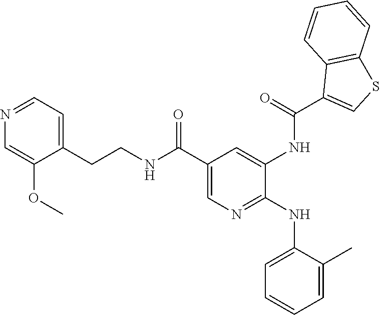 [SMILES: COc1cnccc1CCNC(=O)c1cnc(Nc2ccccc2C)c(NC(=O)c2csc3ccccc23)c1] | | | B | D |
| I- 516 | 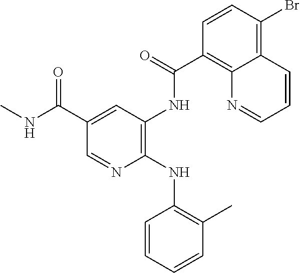 [SMILES: CNC(=O)c1cnc(Nc2ccccc2C)c(NC(=O)c2ccc(Br)c3cccnc23)c1] | | | E | |
| I- 517 | 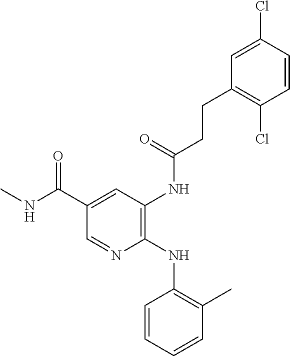 [SMILES: CNC(=O)c1cnc(Nc2ccccc2C)c(NC(=O)CCc2cc(Cl)ccc2Cl)c1] | | | E | |
| I- 518 | 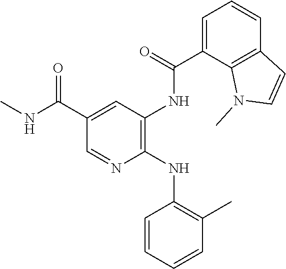 [SMILES: CNC(=O)c1cnc(Nc2ccccc2C)c(NC(=O)c2cccc3ccn(C)c23)c1] | | | E | |
| I- 519 | 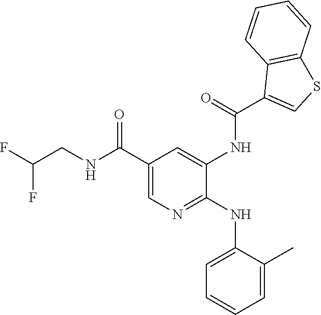 [SMILES: Cc1ccccc1Nc1ncc(C(=O)NCC(F)F)cc1NC(=O)c1csc2ccccc12] | | 467 | A | C |
| I- 520 | 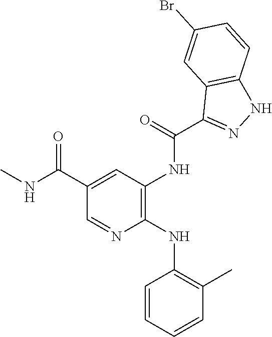 [SMILES: CNC(=O)c1cnc(Nc2ccccc2C)c(NC(=O)c2n[nH]c3ccc(Br)cc23)c1] | | | A | D |
| I- 521 | 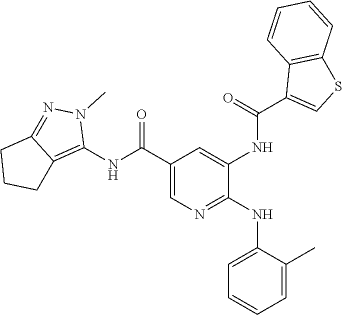 [SMILES: Cc1ccccc1Nc1ncc(C(=O)Nc2c3c(nn2C)CCC3)cc1NC(=O)c1csc2ccccc12] | | | E | |
| I- 522 | 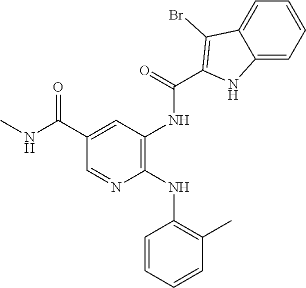 [SMILES: CNC(=O)c1cnc(Nc2ccccc2C)c(NC(=O)c2[nH]c3ccccc3c2Br)c1] | | | E | |
| I- 523 | 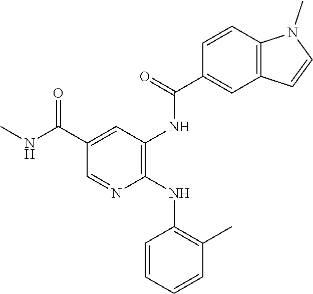 [SMILES: CNC(=O)c1cnc(Nc2ccccc2C)c(NC(=O)c2ccc3c(ccn3C)c2)c1] | | | E | |
| I- 524 | 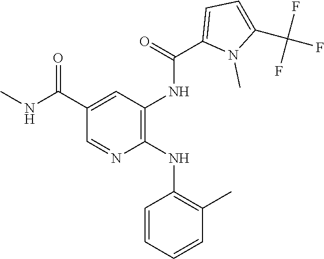 [SMILES: CNC(=O)c1cnc(Nc2ccccc2C)c(NC(=O)c2ccc(C(F)(F)F)n2C)c1] | | | E | |
| I- 525 | 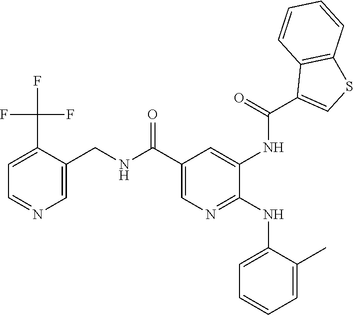 [SMILES: Cc1ccccc1Nc1ncc(C(=O)NCc2cnccc2C(F)(F)F)cc1NC(=O)c1csc2ccccc12] | | | A | D |
| I- 526 | 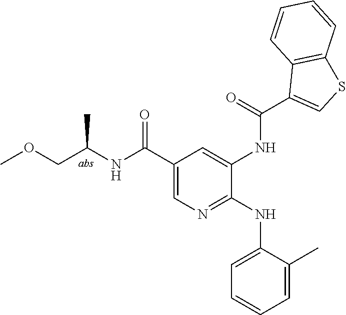 [SMILES: COC[C@@H](C)NC(=O)c1cnc(Nc2ccccc2C)c(NC(=O)c2csc3ccccc23)c1] | | | D | D |
| I- 527 | 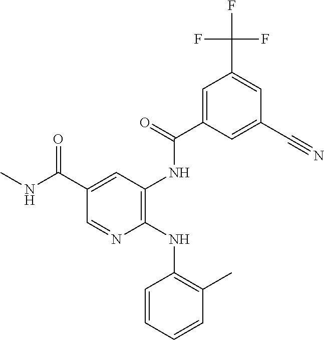 [SMILES: CNC(=O)c1cnc(Nc2ccccc2C)c(NC(=O)c2cc(C#N)cc(C(F)(F)F)c2)c1] | | | B | C |
| I- 528 | 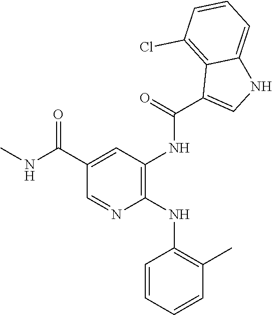 [SMILES: CNC(=O)c1cnc(Nc2ccccc2C)c(NC(=O)c2c[nH]c3cccc(Cl)c23)c1] | | | E | |
| I- 529 | 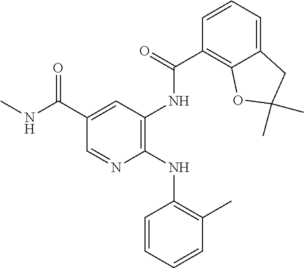 | | | E | |
| I- 530 | 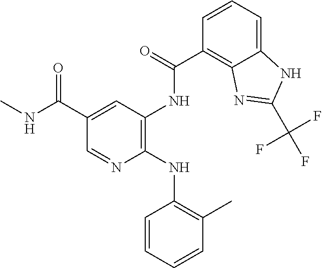 [SMILES: CNC(=O)c1cnc(Nc2ccccc2C)c(NC(=O)c2cccc3[nH]c(C(F)(F)F)nc23)c1] | | | B | D |
| I- 531 | 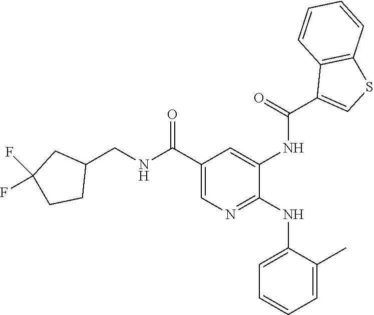 [SMILES: Cc1ccccc1Nc1ncc(C(=O)NCC2CCC(F)(F)C2)cc1NC(=O)c1csc2ccccc12] | | | E | |
| I- 532 | 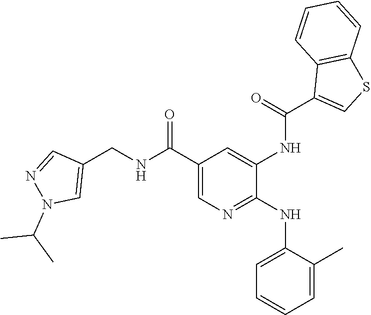 [SMILES: Cc1ccccc1Nc1ncc(C(=O)NCc2cnn(C(C)C)c2)cc1NC(=O)c1csc2ccccc12] | | | B | D |
| I- 533 | 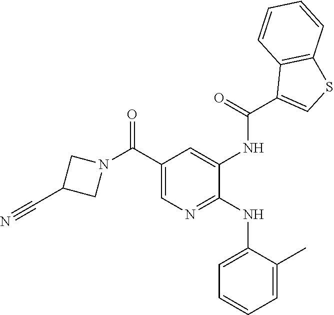 [SMILES: Cc1ccccc1Nc1ncc(C(=O)N2CC(C#N)C2)cc1NC(=O)c1csc2ccccc12] | | | D | D |
| I- 534 | 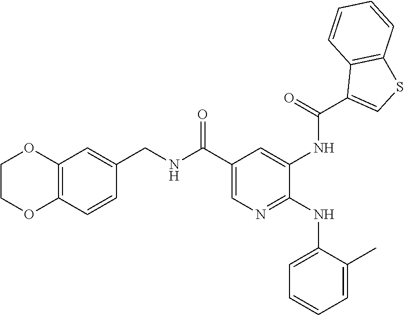 [SMILES: Cc1ccccc1Nc1ncc(C(=O)NCc2ccc3c(c2)OCCO3)cc1NC(=O)c1csc2ccccc12] | | | A | D |
| I- 535 | 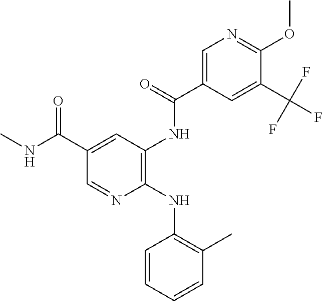 [SMILES: CNC(=O)c1cnc(Nc2ccccc2C)c(NC(=O)c2cnc(OC)c(C(F)(F)F)c2)c1] | 1H-NMR (400 MHz, DMSO-d6) 10.20 (s, 1H), 9.06 (d, J = 2.1 Hz, 1H), 8.66 (d, J = 1.9 Hz, 1H), 8.42 (d, J = 2.2 Hz, 1H), 8.29 (q, J = 4.3 Hz, 1H), 8.25 (s, 1H), 7.99 (d, J = 2.2 Hz, 1H), 7.33 (dd, J = 7.9, 0.9 Hz, 1H), 7.24-7.21 (m, 1H), 7.20-7.15 (m, 1H), 7.08 (td, J = 7.4, 1.3 Hz, 1H), 4.09 (s, 3H), 2.76 (d, J = 4.5 Hz, 3H), 2.15 (s, 3H). | 460.3 | D | D |
| I- 536 | 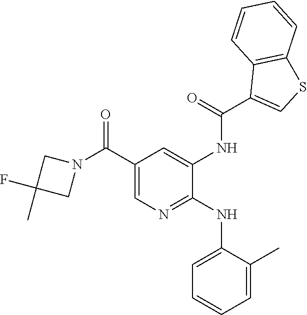 [SMILES: Cc1ccccc1Nc1ncc(C(=O)N2CC(C)(F)C2)cc1NC(=O)c1csc2ccccc12] | | | D | |
| I- 537 | 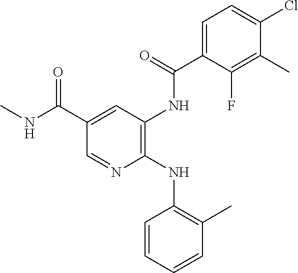 [SMILES: CNC(=O)c1cnc(Nc2ccccc2C)c(NC(=O)c2ccc(Cl)c(C)c2F)c1] | | | E | |
| I- 538 | 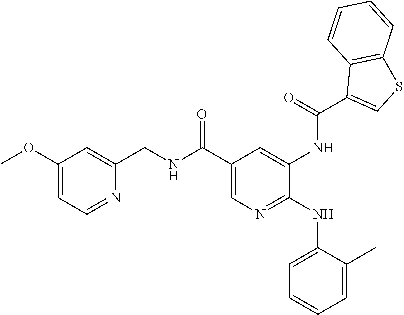 [SMILES: COc1ccnc(CNC(=O)c2cnc(Nc3ccccc3C)c(NC(=O)c3csc4ccccc34)c2)c1] | | | B | D |
| I- 539 | 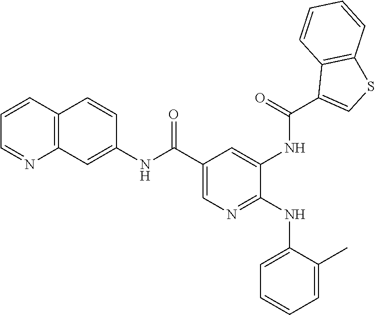 [SMILES: Cc1ccccc1Nc1ncc(C(=O)Nc2ccc3cccnc3c2)cc1NC(=O)c1csc2ccccc12] | | | C | E |
| I- 540 | 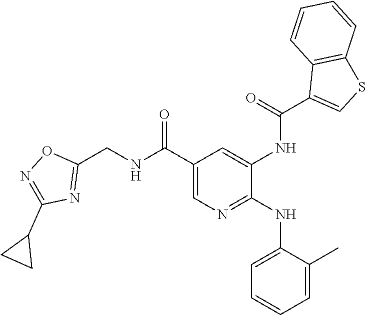 [SMILES: Cc1ccccc1Nc1ncc(C(=O)NCc2nc(C3CC3)no2)cc1NC(=O)c1csc2ccccc12] | | | A | B |
| I- 541 | 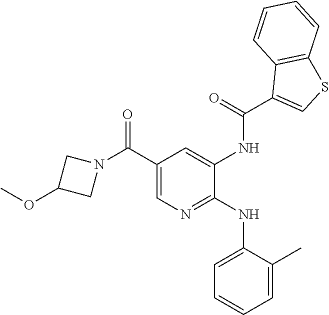 [SMILES: COC1CN(C(=O)c2cnc(Nc3ccccc3C)c(NC(=O)c3csc4ccccc34)c2)C1] | | | D | |
| I- 542 | 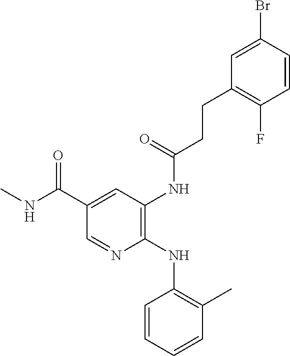 [SMILES: CNC(=O)c1cnc(Nc2ccccc2C)c(NC(=O)CCc2cc(Br)ccc2F)c1] | | | E | |
| I- 543 | 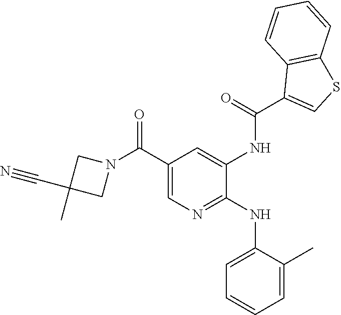 [SMILES: Cc1ccccc1Nc1ncc(C(=O)N2CC(C)(C#N)C2)cc1NC(=O)c1csc2ccccc12] | | | D | |
| I- 544 | 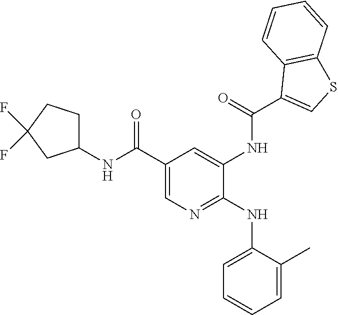 [SMILES: Cc1ccccc1Nc1ncc(C(=O)NC2CCC(F)(F)C2)cc1NC(=O)c1csc2ccccc12] | | | B | D |
| I- 545 | 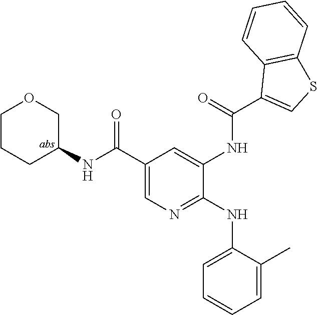 [SMILES: Cc1ccccc1Nc1ncc(C(=O)N[C@H]2CCCOC2)cc1NC(=O)c1csc2ccccc12] | | | B | B |
| I- 546 | 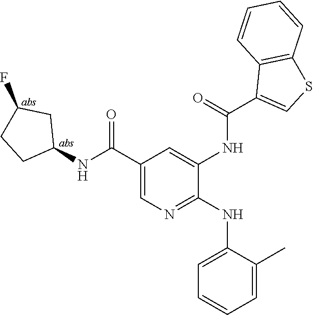 [SMILES: Cc1ccccc1Nc1ncc(C(=O)N[C@H]2CC[C@@H](F)C2)cc1NC(=O)c1csc2ccccc12] | | | A | C |
| I- 547 | 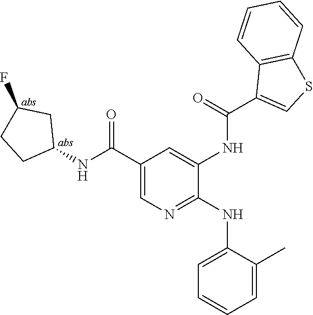 [SMILES: Cc1ccccc1Nc1ncc(C(=O)N[C@@H]2CC[C@@H](F)C2)cc1NC(=O)c1csc2ccccc12] | | | B | C |
| I- 548 | 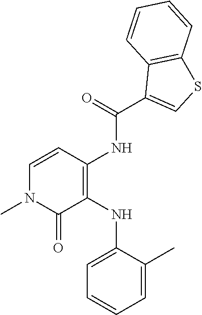 [SMILES: Cc1ccccc1Nc1c(NC(=O)c2csc3ccccc23)ccn(C)c1=O] | (400 MHz, dmso) 9.51 (s, 1H), 8.23 (td, J = 4.5, 2.7 Hz, 1H), 8.05-8.01 (m, 1H), 7.97 (s, 1H), 7.54 (d, J = 7.5 Hz, 1H), 7.44-7.39 (m, 2H), 7.04 (d, J = 6.9 Hz, 1H), 6.90 (dd, J = 10.7, 4.4 Hz, 2H), 6.63 (td, J = 7.2, 1.0 Hz, 1H), 6.36-6.29 (m, 2H), 3.51 (s, 3H), 2.26 (s, 3H). | 390.3 | D | |
| I- 549 | 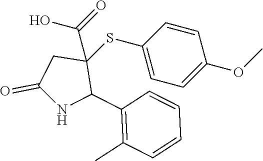 [SMILES: COc1ccc(SC2(C(=O)O)CC(=O)NC2c2ccccc2C)cc1] | (400 MHz, dmso) 13.43 (s, 1H), 8.24 (s, 1H), 7.30-7.21 (m, 3H), 7.19-7.14 (m, 1H), 7.13-7.10 (m, 2H), 6.85-6.80 (m, 2H), 5.38 (d, J = 1.1 Hz, 1H), 3.71 (s, 3H), (400 MHz, dmso) 2.71 (d, J = 16.6 Hz, 1H), 2.59 (d, J = 16.6 Hz, 1H), 2.48 (s, 3H) | 356.3 | D | |
| I- 550 | 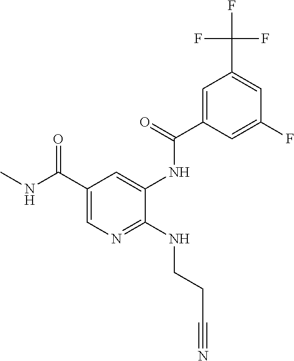 [SMILES: CNC(=O)c1cnc(NCCC#N)c(NC(=O)c2cc(F)cc(C(F)(F)F)c2)c1] | (400 MHz, DMSO) 8.51 (d, J = 2.2 Hz, 1H), 8.37 (s, 1H), 8.26 (q, J = 2.2 Hz, 1H), 8.23 (s, 1H), 8.16 (d, J = 9.0 Hz, 1H), 7.99 (d, J = 8.4 Hz, 1H), 7.90 (d, J = 2.1 Hz, 1H), 7.22 (t, J = 5.6 Hz, 1H), 3.63 (dd, J = 12.5, 6.4 Hz, 2H), 2.79 (t, J = 6.6 Hz, 2H), 2.75 (d, J = 4.5 Hz, 3H). | 410.3 | D | |
| I- 551 | 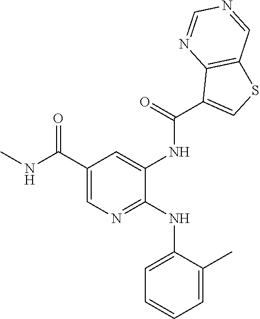 [SMILES: CNC(=O)c1cnc(Nc2ccccc2C)c(NC(=O)c2csc3cncnc23)c1] | (400 MHz, dmso) 10.80 (s, 1H), 9.72 (s, 1H), 9.31 (s, 1H), 9.28 (s, 1H), 8.45 (d, J = 2.2 Hz, 1H), 8.36 (q, J = 4.3 Hz, 1H), 8.30 (s, 1H), 8.30 (s, 1H), 7.29 (dd, J = 7.9, 1.0 Hz, 1H), 7.18 (d, J = 7.4 Hz, 1H), 7.11 (td, J = 7.5, 1.2 Hz, 1H), 6.99 (td, J = 7.4, 1.3 Hz, 1H), 2.78 (d, J = 4.5 Hz, 3H), 2.20 (s, 3H). | 419.3 | D | |
| I- 552 | 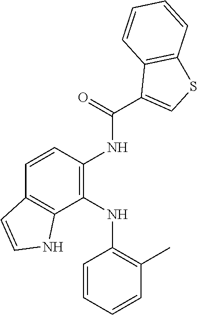 [SMILES: Cc1ccccc1Nc1c(NC(=O)c2csc3ccccc23)ccc2cc[nH]c12] | (400 MHz, dmso) 10.81 (s, 1H), 10.09 (s, 1H), 8.40-8.31 (m, 1H), 8.28 (s, 1H), 8.10-8.02 (m, 1H), 7.50-7.39 (m, 3H), 7.27 (t, J = 4.0 Hz, 1H), 7.24 (d, J = 8.4 Hz, 1H), 7.04 (d, J = 6.9 Hz, 1H), 6.83 (t, J = 7.4 Hz, 1H), 6.71 (s, 1H), 6.58 (t, J = 7.3 Hz, 1H), 6.48 (dd, J = 3.0, 1.9 Hz, 1H), 6.00 (d, J = 8.0 Hz, 1H), 2.28 (s, 3H). | 398.3 | D | |
| I- 553 | 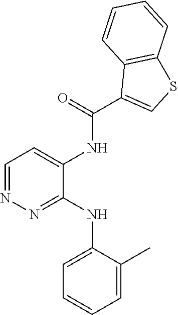 [SMILES: Cc1ccccc1Nc1nnccc1NC(=O)c1csc2ccccc12] | (400 MHz, dmso) 8.72 (s, 1H), 8.58- 8.48 (m, 2H), 8.40-8.24 (m, 1H), 8.09 (d, J = 7.5 Hz, 1H), 7.95 (s, 1H), 7.55- 7.42 (m, 2H), 7.28-7.16 (m, 2H), 7.06 (t, J = 7.3 Hz, 1H), 2.20 (s, 3H). | 361.3 | D | |
| I- 554 | 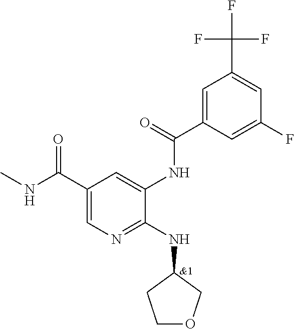 [SMILES: CNC(=O)c1cnc(N[C@@H]2CCOC2)c(NC(=O)c2cc(F)cc(C(F)(F)F)c2)c1] | (400 MHz, DMSO) 10.01 (s, 1H), 8.49 (d, J = 2.2 Hz, 1H), 8.22 (m, 2H), 8.16 (d, J = 9.1 Hz, 1H), 7.99 (d, J = 8.4 Hz, 1H), 7.90 (d, J = 2.2 Hz, 1H), 6.79 (d, J = 6.2 Hz, 1H), 4.67-4.55 (m, 1H), 3.91 (dd, J = 8.9, 6.4 Hz, 1H), 3.85 (td, J = 8.0, 6.3 Hz, 1H), 3.69 (td, J = 7.9, 6.5 Hz, 1H), 3.57 (dd, J = 8.9, 4.4 Hz, 1H), 2.75 (d, J = 4.5 Hz, 3H), 2.20 (ddt, J = 15.5, 14.0, 7.0 Hz, 1H), 1.96-1.84 (m, 1H). | 427.3 | D | |
| I- 555 | 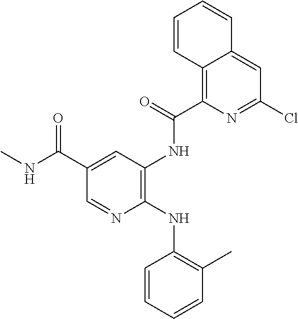 [SMILES: CNC(=O)c1cnc(Nc2ccccc2C)c(NC(=O)c2nc(Cl)cc3ccccc23)c1] | | | D | |
| I- 556 | 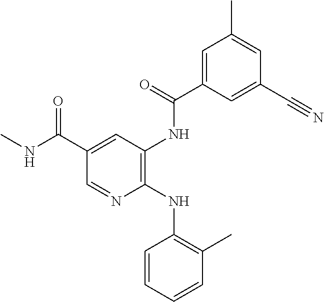 [SMILES: CNC(=O)c1cnc(Nc2ccccc2C)c(NC(=O)c2cc(C)cc(C#N)c2)c1] | | | D | |
| I- 557 | 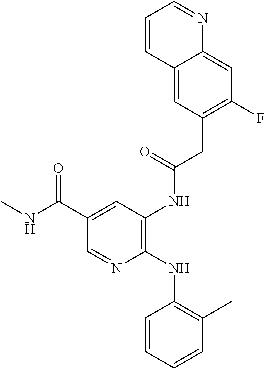 [SMILES: CNC(=O)c1cnc(Nc2ccccc2C)c(NC(=O)Cc2cc3cccnc3cc2F)c1] | | | D | |
| I- 558 | 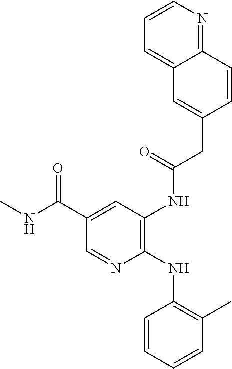 [SMILES: CNC(=O)c1cnc(Nc2ccccc2C)c(NC(=O)Cc2ccc3ncccc3c2)c1] | | | D | |
| I- 559 | 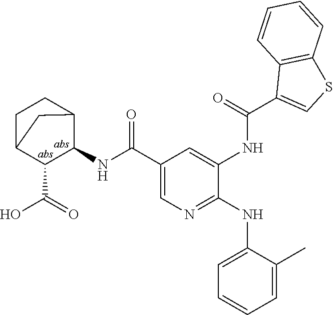 [SMILES: Cc1ccccc1Nc1ncc(C(=O)N[C@@H]2C3CCC(C3)[C@H]2C(=O)O)cc1NC(=O)c1csc2ccccc12] | | | D | |
| I- 560 | 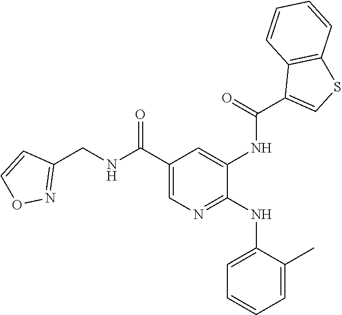 [SMILES: Cc1ccccc1Nc1ncc(C(=O)NCc2ccon2)cc1NC(=O)c1csc2ccccc12] | | | A | B |
| I- 561 | 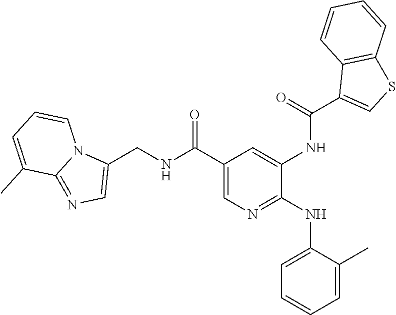 [SMILES: Cc1ccccc1Nc1ncc(C(=O)NCc2cnc3c(C)cccn23)cc1NC(=O)c1csc2ccccc12] | | | B | D |
| I- 562 | 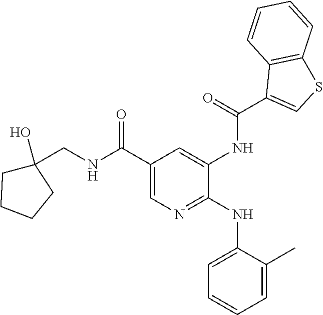 [SMILES: Cc1ccccc1Nc1ncc(C(=O)NCC2(O)CCCC2)cc1NC(=O)c1csc2ccccc12] | | | C | |
| I- 563 | 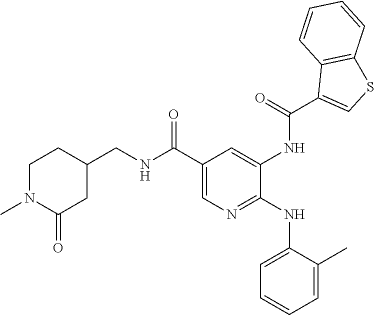 [SMILES: Cc1ccccc1Nc1ncc(C(=O)NCC2CCN(C)C(=O)C2)cc1NC(=O)c1csc2ccccc12] | | | B | D |
| I- 564 | 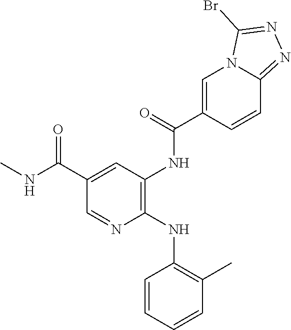 [SMILES: CNC(=O)c1cnc(Nc2ccccc2C)c(NC(=O)c2ccc3nnc(Br)n3c2)c1] | | | C | D |
| I- 565 | 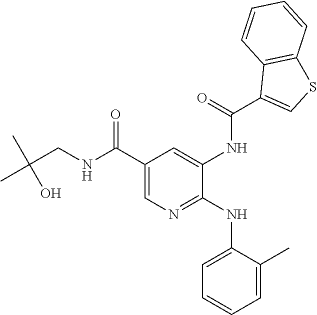 [SMILES: Cc1ccccc1Nc1ncc(C(=O)NCC(C)(C)O)cc1NC(=O)c1csc2ccccc12] | | | D | |
| I- 566 | 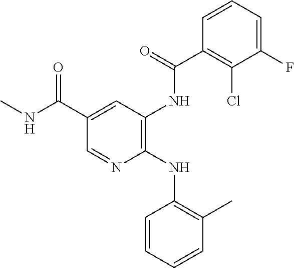 [SMILES: CNC(=O)c1cnc(Nc2ccccc2C)c(NC(=O)c2cccc(F)c2Cl)c1] | | | D | |
| I- 567 | 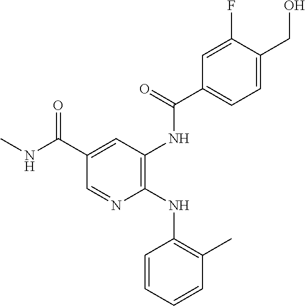 [SMILES: CNC(=O)c1cnc(Nc2ccccc2C)c(NC(=O)c2ccc(CO)c(F)c2)c1] | | | D | |
| I- 568 | 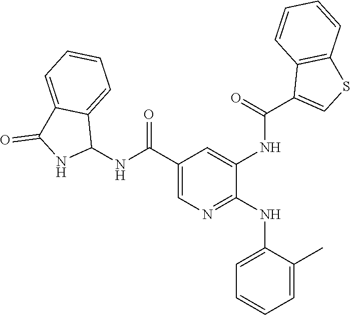 [SMILES: Cc1ccccc1Nc1ncc(C(=O)NC2NC(=O)c3ccccc32)cc1NC(=O)c1csc2ccccc12] | | | A | B |
| I- 569 | 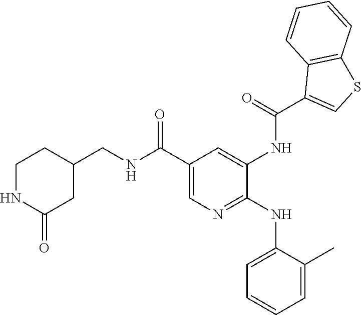 [SMILES: Cc1ccccc1Nc1ncc(C(=O)NCC2CCNC(=O)C2)cc1NC(=O)c1csc2ccccc12] | | | B | D |
| I- 570 | 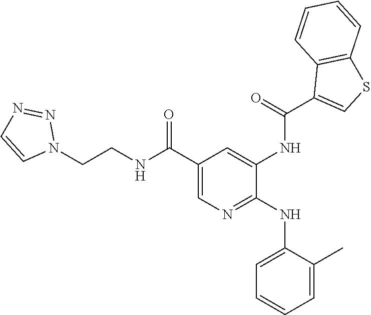 [SMILES: Cc1ccccc1Nc1ncc(C(=O)NCCn2ccnn2)cc1NC(=O)c1csc2ccccc12] | | | B | D |
| I- 571 | 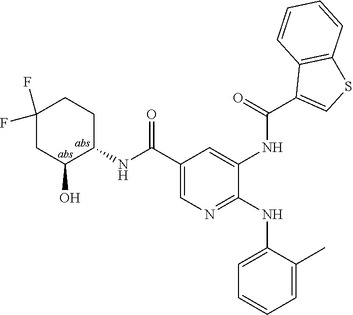 [SMILES: Cc1ccccc1Nc1ncc(C(=O)N[C@H]2CCC(F)(F)C[C@@H]2O)cc1NC(=O)c1csc2ccccc12] | | | B | B |
| I- 572 | 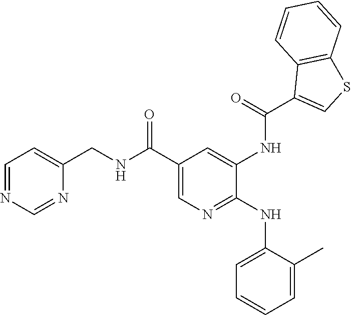 [SMILES: Cc1ccccc1Nc1ncc(C(=O)NCc2ccncn2)cc1NC(=O)c1csc2ccccc12] | | | B | A |
| I- 573 | 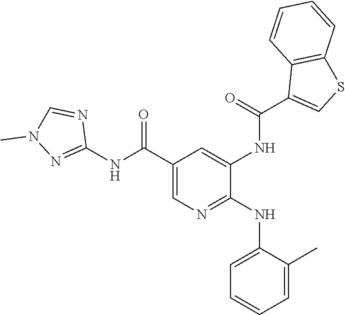 [SMILES: Cc1ccccc1Nc1ncc(C(=O)Nc2ncn(C)n2)cc1NC(=O)c1csc2ccccc12] | | | C | D |
| I- 574 | 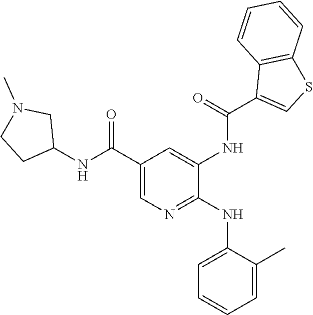 [SMILES: Cc1ccccc1Nc1ncc(C(=O)NC2CCN(C)C2)cc1NC(=O)c1csc2ccccc12] | | | D | |
| I- 575 | 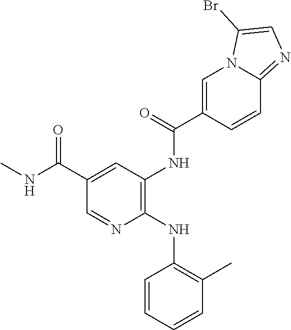 [SMILES: CNC(=O)c1cnc(Nc2ccccc2C)c(NC(=O)c2ccc3ncc(Br)n3c2)c1] | | | C | |
| I- 576 | 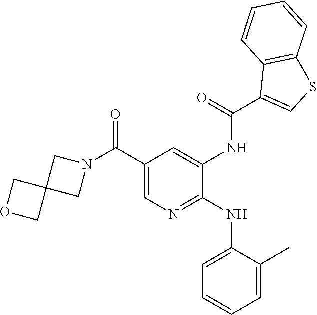 [SMILES: Cc1ccccc1Nc1ncc(C(=O)N2CC3(COC3)C2)cc1NC(=O)c1csc2ccccc12] | | | D | |
| I- 577 | 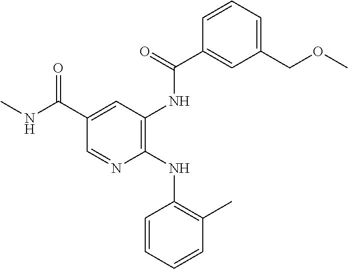 | | | D | |
| I- 578 | 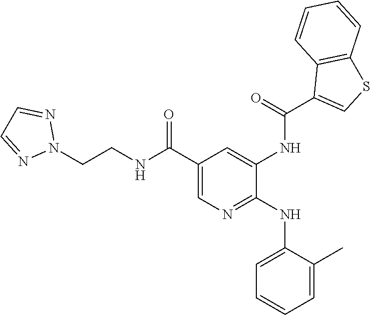 [SMILES: Cc1ccccc1Nc1ncc(C(=O)NCCn2nccn2)cc1NC(=O)c1csc2ccccc12] | | | B | D |
| I- 579 | 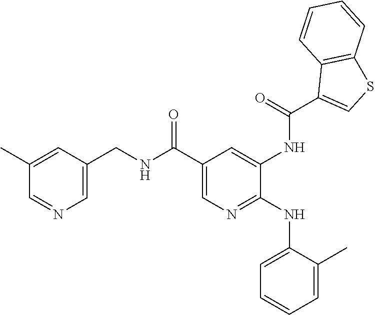 [SMILES: Cc1cncc(CNC(=O)c2cnc(Nc3ccccc3C)c(NC(=O)c3csc4ccccc34)c2)c1] | | | A | C |
| I- 580 | 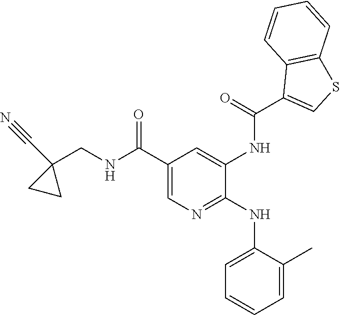 [SMILES: Cc1ccccc1Nc1ncc(C(=O)NCC2(C#N)CC2)cc1NC(=O)c1csc2ccccc12] | | | A | B |
| I- 581 | 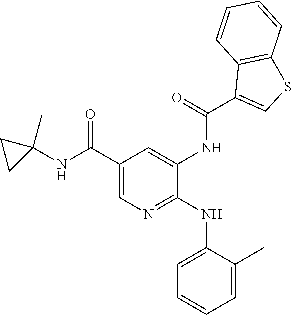 [SMILES: Cc1ccccc1Nc1ncc(C(=O)NC2(C)CC2)cc1NC(=O)c1csc2ccccc12] | | | A | B |
| I- 582 | 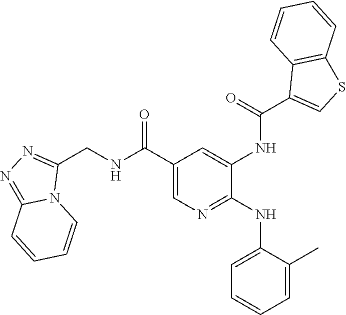 [SMILES: Cc1ccccc1Nc1ncc(C(=O)NCc2nnc3ccccn23)cc1NC(=O)c1csc2ccccc12] | | | A | C |
| I- 583 | 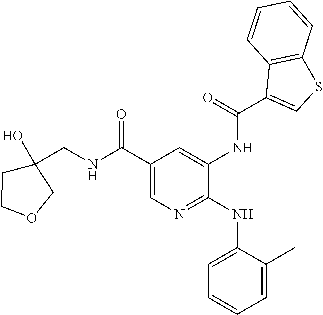 [SMILES: Cc1ccccc1Nc1ncc(C(=O)NCC2(O)CCOC2)cc1NC(=O)c1csc2ccccc12] | | | C | D |
| I- 584 | 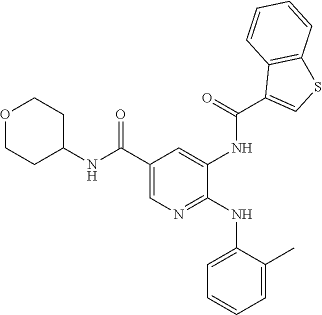 [SMILES: Cc1ccccc1Nc1ncc(C(=O)NC2CCOCC2)cc1NC(=O)c1csc2ccccc12] | | | B | C |
| I- 585 | 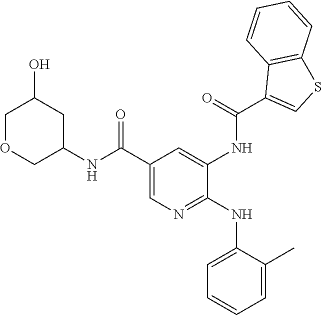 [SMILES: Cc1ccccc1Nc1ncc(C(=O)NC2COCC(O)C2)cc1NC(=O)c1csc2ccccc12] | | | B | D |
| I- 586 | 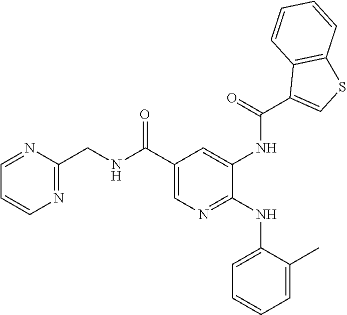 [SMILES: Cc1ccccc1Nc1ncc(C(=O)NCc2ncccn2)cc1NC(=O)c1csc2ccccc12] | | | C | |
| I- 587 | 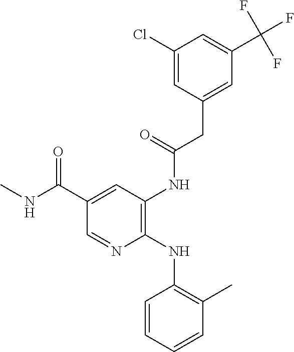 [SMILES: CNC(=O)c1cnc(Nc2ccccc2C)c(NC(=O)Cc2cc(Cl)cc(C(F)(F)F)c2)c1] | | | D | |
| I- 588 | 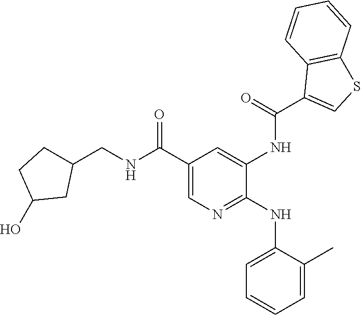 [SMILES: Cc1ccccc1Nc1ncc(C(=O)NCC2CCC(O)C2)cc1NC(=O)c1csc2ccccc12] | | | B | D |
| I- 589 | 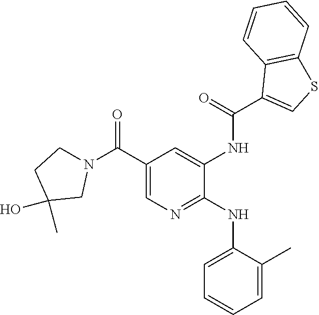 [SMILES: Cc1ccccc1Nc1ncc(C(=O)N2CCC(C)(O)C2)cc1NC(=O)c1csc2ccccc12] | | | D | |
| I- 590 | 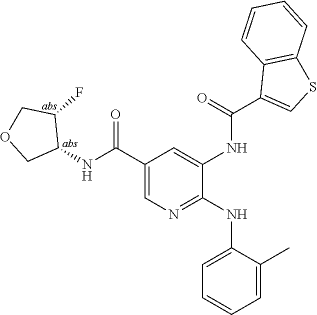 [SMILES: Cc1ccccc1Nc1ncc(C(=O)N[C@@H]2COC[C@@H]2F)cc1NC(=O)c1csc2ccccc12] | | | B | C |
| I- 591 | 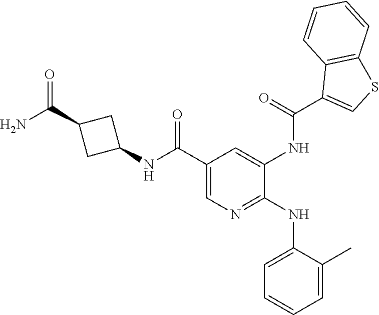 [SMILES: Cc1ccccc1Nc1ncc(C(=O)N[C@H]2C[C@@H](C(N)=O)C2)cc1NC(=O)c1csc2ccccc12] | (400 MHz, dmso) 10.03 (Br s, 1H), 8.67 (s, 1H), 8.52 (d, J = 7.7 Hz, 1H), 8.46 (dd, J = 7.0, 1.2 Hz, 1H), 8.42 (d, J = 2.2 Hz, 1H), 8.18 (s, 1H), 8.12 (d, J = 2.2 Hz, 1H), 8.06 (dd, J = 7.0, 1.2 Hz, 1H), 7.51-7.36 (m, 3H), 7.19-7.18 (m, 2H), 7.14 (t, J = 7.7 Hz, 1H), 7.02 (td, J = 7.7, 1.2 Hz, 1H), 6.71 (Br s, 1H), 4.38-4.22 (m, 1H), 2.67-2.56 (m, 1H), 2.34-2.25 (m, 2H), 2.19-2.09 (m, 5H). | 500.3 | A | B |
| I- 592 | 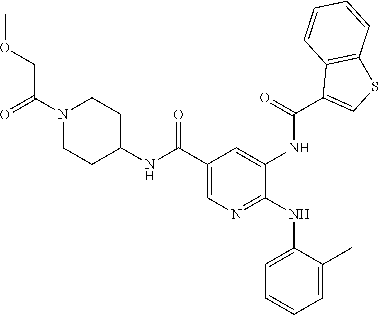 [SMILES: COCC(=O)N1CCC(NC(=O)c2cnc(Nc3ccccc3C)c(NC(=O)c3csc4ccccc34)c2)CC1] | | | D | C |
| I- 593 | 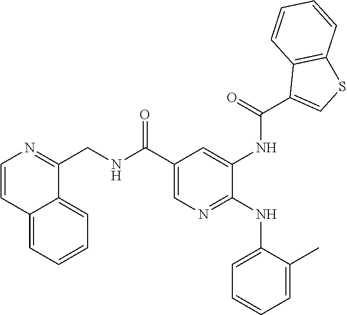 [SMILES: Cc1ccccc1Nc1ncc(C(=O)NCc2nccc3ccccc23)cc1NC(=O)c1csc2ccccc12] | | | B | D |
| I- 594 | 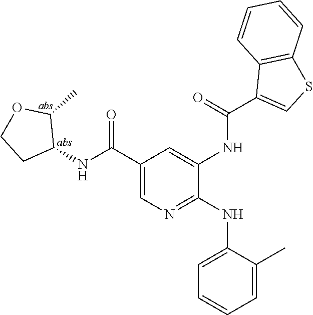 [SMILES: Cc1ccccc1Nc1ncc(C(=O)N[C@@H]2CCO[C@@H]2C)cc1NC(=O)c1csc2ccccc12] | | | B | C |
| I- 595 | 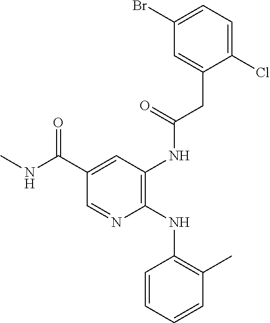 [SMILES: CNC(=O)c1cnc(Nc2ccccc2C)c(NC(=O)Cc2cc(Br)ccc2Cl)c1] | | | D | |
| I- 596 | 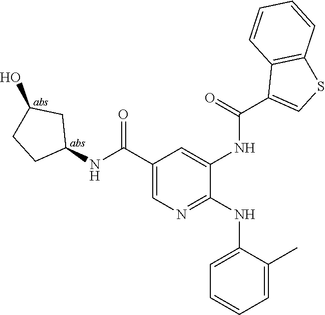 [SMILES: Cc1ccccc1Nc1ncc(C(=O)N[C@H]2CC[C@@H](O)C2)cc1NC(=O)c1csc2ccccc12] | | | B | B |
| I- 597 | 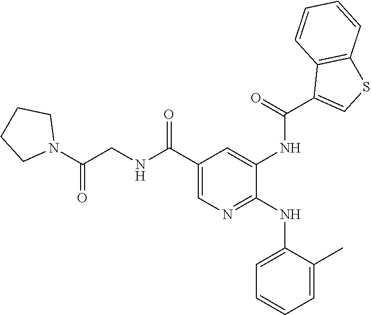 [SMILES: Cc1ccccc1Nc1ncc(C(=O)NCC(=O)N2CCCC2)cc1NC(=O)c1csc2ccccc12] | | | D | E |
| I- 598 | 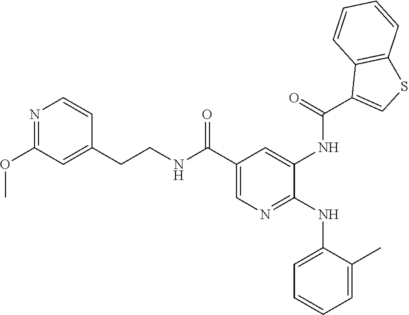 [SMILES: COc1cc(CCNC(=O)c2cnc(Nc3ccccc3C)c(NC(=O)c3csc4ccccc34)c2)ccn1] | | | B | C |
| I- 599 | 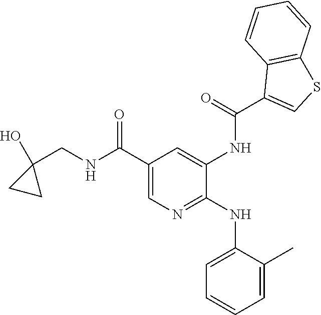 [SMILES: Cc1ccccc1Nc1ncc(C(=O)NCC2(O)CC2)cc1NC(=O)c1csc2ccccc12] | | | B | C |
| I- 600 | 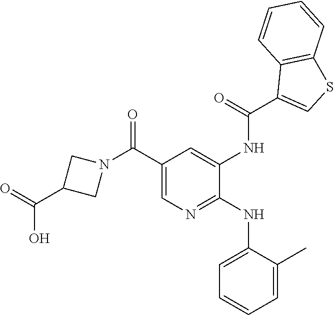 [SMILES: Cc1ccccc1Nc1ncc(C(=O)N2CC(C(=O)O)C2)cc1NC(=O)c1csc2ccccc12] | | | D | |
| I- 601 | 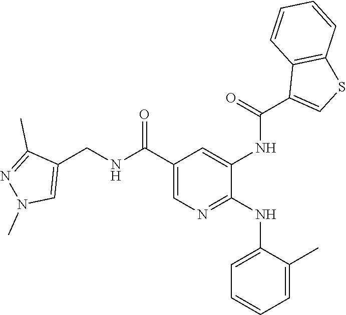 [SMILES: Cc1ccccc1Nc1ncc(C(=O)NCc2cn(C)nc2C)cc1NC(=O)c1csc2ccccc12] | | | A | C |
| I- 602 | 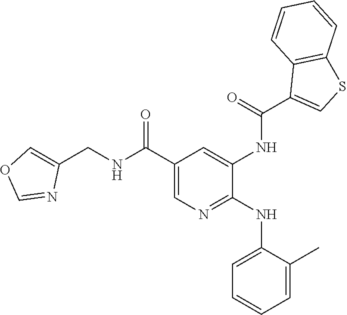 [SMILES: Cc1ccccc1Nc1ncc(C(=O)NCc2cocn2)cc1NC(=O)c1csc2ccccc12] | | | B | D |
| I- 603 | 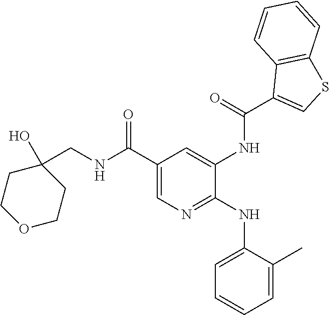 [SMILES: Cc1ccccc1Nc1ncc(C(=O)NCC2(O)CCOCC2)cc1NC(=O)c1csc2ccccc12] | | | C | D |
| I- 604 | 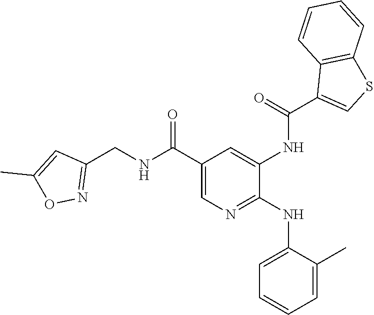 [SMILES: Cc1cc(CNC(=O)c2cnc(Nc3ccccc3C)c(NC(=O)c3csc4ccccc34)c2)no1] | | | A | B |
| I- 605 | 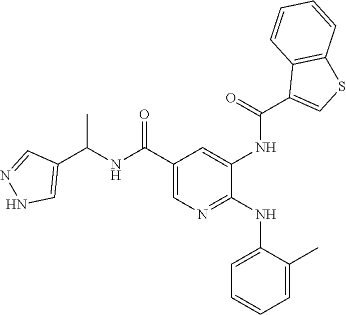 [SMILES: Cc1ccccc1Nc1ncc(C(=O)NC(C)c2cn[nH]c2)cc1NC(=O)c1csc2ccccc12] | | | A | B |
| I- 606 | 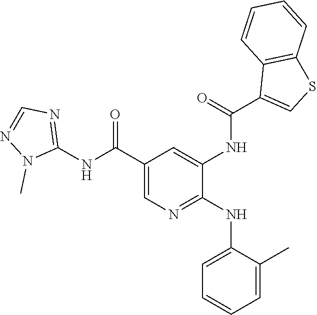 [SMILES: Cc1ccccc1Nc1ncc(C(=O)Nc2ncnn2C)cc1NC(=O)c1csc2ccccc12] | | | D | |
| I- 607 | 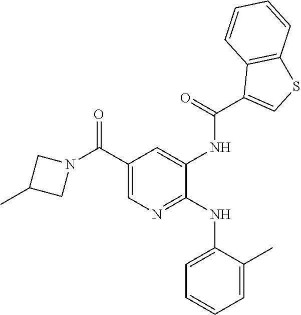 [SMILES: Cc1ccccc1Nc1ncc(C(=O)N2CC(C)C2)cc1NC(=O)c1csc2ccccc12] | | | B | C |
| I- 608 | 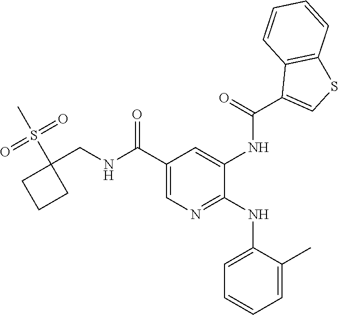 [SMILES: Cc1ccccc1Nc1ncc(C(=O)NCC2(S(C)(=O)=O)CCC2)cc1NC(=O)c1csc2ccccc12] | | | C | D |
| I- 609 | 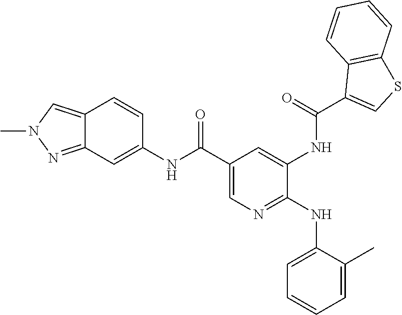 [SMILES: Cc1ccccc1Nc1ncc(C(=O)Nc2ccc3cn(C)nc3c2)cc1NC(=O)c1csc2ccccc12] | | | A | B |
| I- 610 | 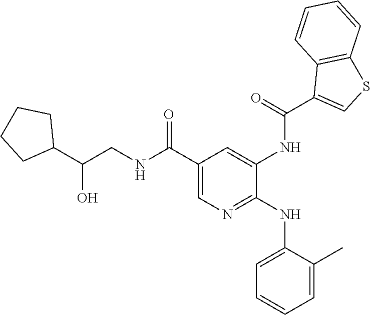 [SMILES: Cc1ccccc1Nc1ncc(C(=O)NCC(O)C2CCCC2)cc1NC(=O)c1csc2ccccc12] | | | B | D |
| I- 611 | 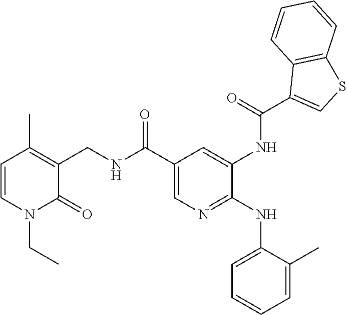 [SMILES: CCn1ccc(C)c(CNC(=O)c2cnc(Nc3ccccc3C)c(NC(=O)c3csc4ccccc34)c2)c1=O] | | | B | C |
| I- 612 | 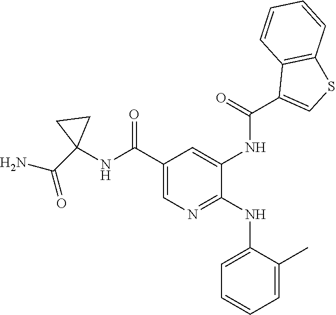 [SMILES: Cc1ccccc1Nc1ncc(C(=O)NC2(C(N)=O)CC2)cc1NC(=O)c1csc2ccccc12] | | | B | A |
| I- 613 | 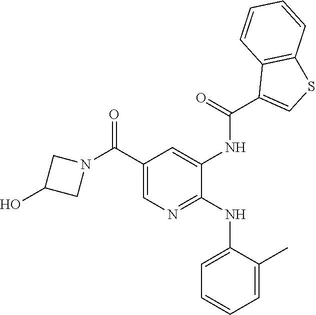 [SMILES: Cc1ccccc1Nc1ncc(C(=O)N2CC(O)C2)cc1NC(=O)c1csc2ccccc12] | | | B | D |
| I- 614 | 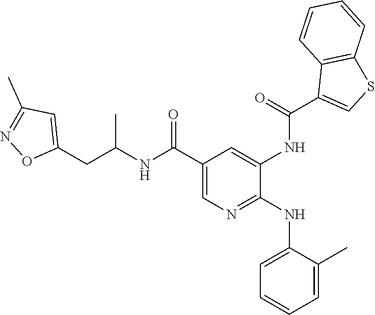 [SMILES: Cc1cc(CC(C)NC(=O)c2cnc(Nc3ccccc3C)c(NC(=O)c3csc4ccccc34)c2)on1] | | | D | |
| I- 615 | 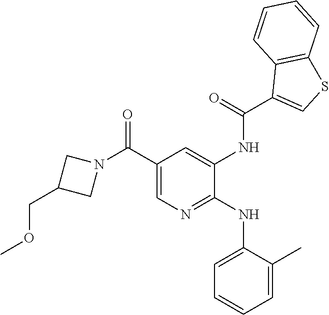 [SMILES: COCC1CN(C(=O)c2cnc(Nc3ccccc3C)c(NC(=O)c3csc4ccccc34)c2)C1] | | | D | |
| I- 616 | 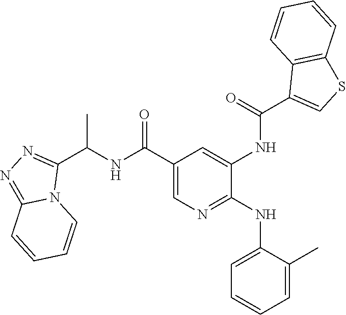 [SMILES: Cc1ccccc1Nc1ncc(C(=O)NC(C)c2nnc3ccccn23)cc1NC(=O)c1csc2ccccc12] | | | A | B |
| I- 617 | 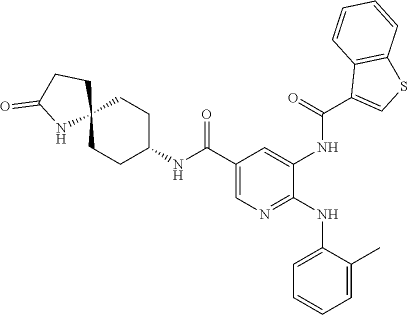 [SMILES: Cc1ccccc1Nc1ncc(C(=O)N[C@H]2CC[C@]3(CCC(=O)N3)CC2)cc1NC(=O)c1csc2ccccc12] | | | D | |
| I- 618 | 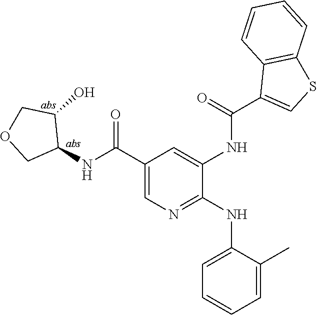 [SMILES: Cc1ccccc1Nc1ncc(C(=O)N[C@H]2COC[C@@H]2O)cc1NC(=O)c1csc2ccccc12] | | | B | B |
| I- 619 | 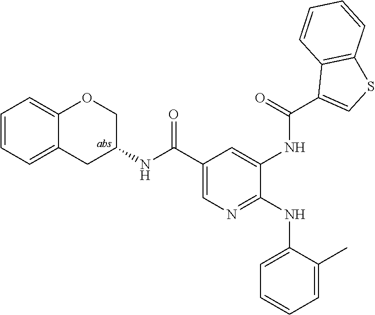 [SMILES: Cc1ccccc1Nc1ncc(C(=O)N[C@H]2COc3ccccc3C2)cc1NC(=O)c1csc2ccccc12] | | | B | E |
| I- 620 | 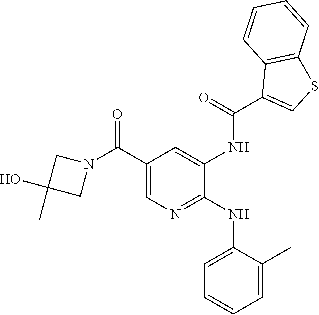 [SMILES: Cc1ccccc1Nc1ncc(C(=O)N2CC(C)(O)C2)cc1NC(=O)c1csc2ccccc12] | | | D | |
| I- 621 | 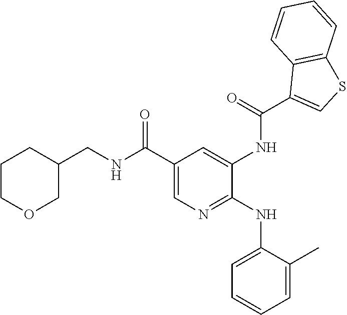 [SMILES: Cc1ccccc1Nc1ncc(C(=O)NCC2CCCOC2)cc1NC(=O)c1csc2ccccc12] | | | B | D |
| I- 622 | 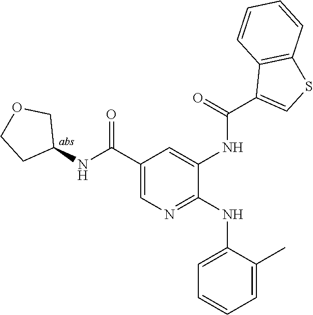 [SMILES: Cc1ccccc1Nc1ncc(C(=O)N[C@H]2CCOC2)cc1NC(=O)c1csc2ccccc12] | | | B | B |
| I- 623 | 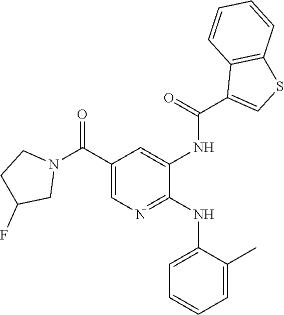 [SMILES: Cc1ccccc1Nc1ncc(C(=O)N2CCC(F)C2)cc1NC(=O)c1csc2ccccc12] | | | C | |
| I- 624 | 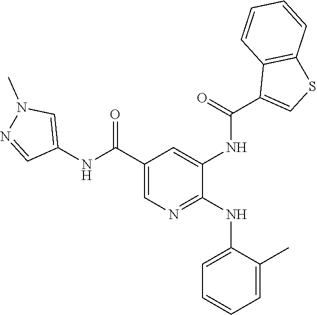 [SMILES: Cc1ccccc1Nc1ncc(C(=O)Nc2cnn(C)c2)cc1NC(=O)c1csc2ccccc12] | | | B | A |
| I- 625 | 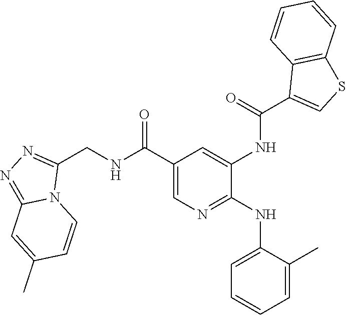 [SMILES: Cc1ccn2c(CNC(=O)c3cnc(Nc4ccccc4C)c(NC(=O)c4csc5ccccc45)c3)nnc2c1] | | | B | D |
| I- 626 | 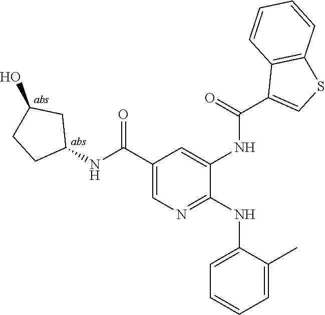 [SMILES: Cc1ccccc1Nc1ncc(C(=O)N[C@@H]2CC[C@@H](O)C2)cc1NC(=O)c1csc2ccccc12] | | | B | B |
| I- 627 | 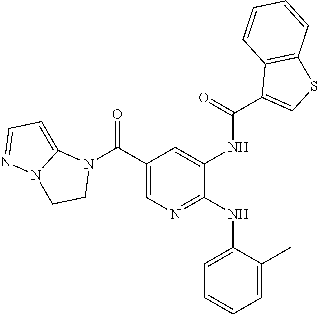 [SMILES: Cc1ccccc1Nc1ncc(C(=O)N2CCn3nccc32)cc1NC(=O)c1csc2ccccc12] | | | D | D |
| I- 628 | 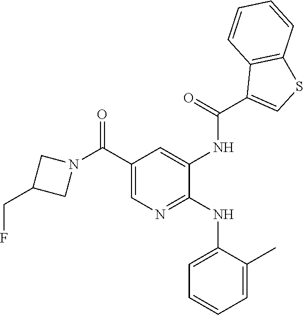 [SMILES: Cc1ccccc1Nc1ncc(C(=O)N2CC(CF)C2)cc1NC(=O)c1csc2ccccc12] | | | D | |
| I- 629 | 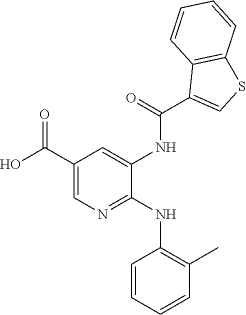 [SMILES: Cc1ccccc1Nc1ncc(C(=O)O)cc1NC(=O)c1csc2ccccc12] | | | D | |
| I- 630 | 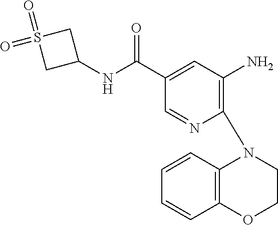 [SMILES: Nc1cc(C(=O)NC2CS(=O)(=O)C2)cnc1N1CCOc2ccccc21] | | | D | |
| I- 631 | 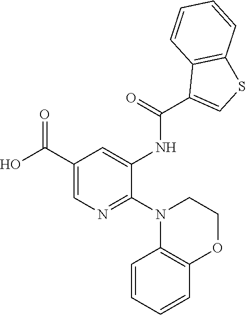 [SMILES: O=C(O)c1cnc(N2CCOc3ccccc32)c(NC(=O)c2csc3ccccc23)c1] | | | D | |
| I- 632 | 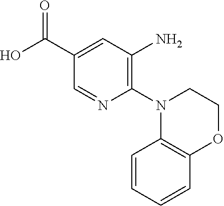 [SMILES: Nc1cc(C(=O)O)cnc1N1CCOc2ccccc21] | | | D | |
| I- 633 | 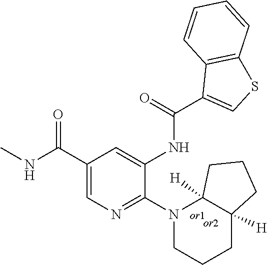 [SMILES: CNC(=O)c1cnc(N2CCC[C@@H]3CCC[C@@H]32)c(NC(=O)c2csc3ccccc23)c1] | | | D | |
| I- 634 | 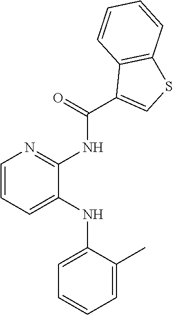 [SMILES: Cc1ccccc1Nc1cccnc1NC(=O)c1csc2ccccc12] | (400 MHz, dmso) 9.75 (s, 1H), 8.57- 8.51 (m, 2H), 8.48-8.42 (m, 1H), 8.38 (q, J = 4.5 Hz, 1H), 8.20 (d, J = 2.2 Hz, 1H), 8.11-8.04 (m, 1H), 7.53-7.39 (m, 2H), 4.16 (dd, J = 13.3, 7.5 Hz, 1H), 3.49 (d, J = 13.3 Hz, 1H), 3.01 (t, J = 11.3 Hz, 1H), 2.76 (d, J = 4.5 Hz, 3H), 1.92 (m, 1H), 1.72-1.27 (m, 10H). | 435 | D | |
| I- 635 | 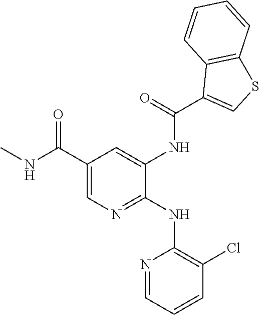 [SMILES: CNC(=O)c1cnc(Nc2ncccc2Cl)c(NC(=O)c2csc3ccccc23)c1] | (400 MHz, DMSO) 10.85 (s, 1H), 8.71 (s, 1H), 8.51 (d, J = 7.9 Hz, 1H), 8.09 (d, J = 7.3 Hz, 1H), 8.04 (d, J = 3.2 Hz, 1H), 7.55-7.36 (m, 4H), 7.25 (dd, J = 7.8, 4.5 Hz, 1H), 7.19 (d, J = 7.4 Hz, 1H), 7.12 (t, J = 7.5 Hz, 1H), 7.07 (s, 1H), 7.01 (d, J = 7.7 Hz, 1H), 6.90 (t, J = 7.2 Hz, 1H), 2.19 (s, 3H). | 359.9 | B | E |
| I- 636 | 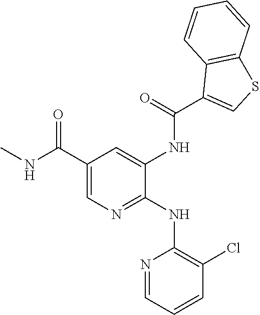 [SMILES: CNC(=O)c1cnc(Nc2ncccc2Cl)c(NC(=O)c2csc3ccccc23)c1] | (400 MHz, DMSO) 7.49 (s, 1H), 7.34 (dd, J = 7.9, 1.0 Hz, 1H), 7.30 (d, J = 3.0 Hz, 1H), 7.22-7.20 (m, 2H), 7.15 (td, J = 7.6, 1.4 Hz, 1H), 7.03 (td, J = 7.4, 1.3 Hz, 1H), 6.21 (s, 2H), 2.15 (s, 3H). | 200.9 | D | |
| I- 637 | 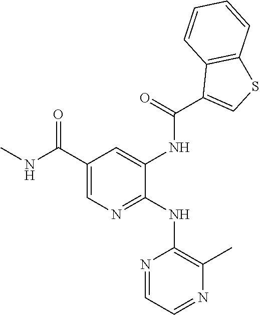 [SMILES: CNC(=O)c1cnc(Nc2nccnc2C)c(NC(=O)c2csc3ccccc23)c1] | (400 MHz, DMSO-d6) 8.72-8.31 (m, 5H), 8.21 (dd, J = 4.8, 1.6 Hz, 1H), 8.06 (d, J = 7.6 Hz, 1H), 7.85 (d, J = 6.7 Hz, 1H), 7.52-7.37 (m, 2H), 7.01 (dd, J = 10.1, 4.3 Hz, 1H), 2.79 (d, J = 4.5 Hz, 3H). | 438.2 | D | |
| I- 638 | 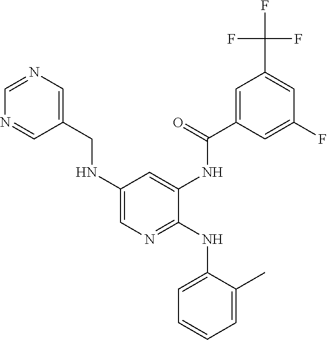 [SMILES: Cc1ccccc1Nc1ncc(NCc2cncnc2)cc1NC(=O)c1cc(F)cc(C(F)(F)F)c1] | (400 MHz, dmso) 10.04 (br s, 1H), 9.16 (br s, 1H), 8.54 (s, 1H), 8.52-8.42 (m, 3H), 8.42-8.32 (m, 1H), 8.15 (s, 2H), 8.12-8.03 (m, 1H), 7.53-7.40 (m, 2H), 2.80 (d, J = 4.5 Hz, 3H), 2.36 (s, 3H). Contains approx. 10% impurities. | 418.9 | D | |
| I- 639 | 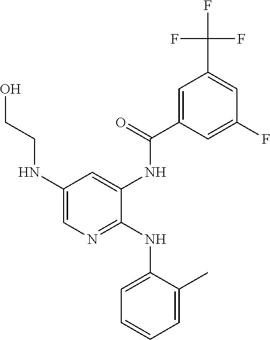 [SMILES: Cc1ccccc1Nc1ncc(NCCO)cc1NC(=O)c1cc(F)cc(C(F)(F)F)c1] | (400 MHz, dmso) 9.06 (s, 1H), 8.84 (s, 2H), 7.82 (s, 1H), 7.82 (s, 1H), 7.68 (m, 2H), 7.62 (d, J = 2.5 Hz, 1H), 7.45 (d, J = 2.7 Hz, 1H), 7.10 (d, J = 7.2 Hz, 1H), 6.98 (t, J = 7.9 Hz, 1H), 6.83 (d, J = 7.5 Hz, 1H), 6.78 (t, J = 7.2 Hz, 1H), 4.45 (s, 2H), 2.22 (s, 3H). | 496.9 | A | C |
| I- 640 | 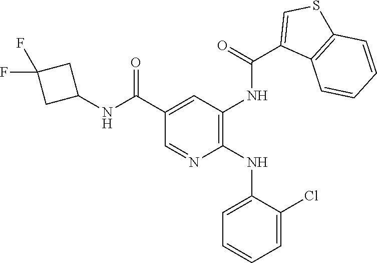 [SMILES: O=C(NC1CC(F)(F)C1)c1cnc(Nc2ccccc2Cl)c(NC(=O)c2csc3ccccc23)c1] | (400 MHz, dmso) 7.91 (d, J = 2.5 Hz, 1H), 7.80-7.74 (m, 1H), 7.59-7.47 (m, 4H), 7.42-7.38 (m, 2H), 7.31 (d, J = 2.5 Hz, 1H), 5.78 (t, J = 5.7 Hz, 1H), 4.75 (t, J = 5.3 Hz, 1H), 3.62 (q, J = 5.8 Hz, 2H), 3.18 (q, J = 5.9 Hz, 2H), 1.89 (s, 3H). | 449 | D | |
| I- 641 | 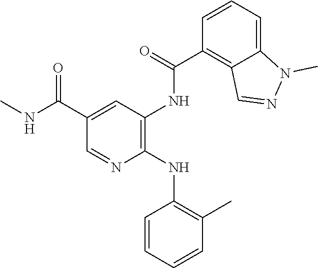 | (400 MHz, DMSO-d6) 10.35-10.45 (m, 1H), 8.82 (d, J = 6.57 Hz, 1H), 8.77 (s, 1H), 8.59 (d, J = 2.27 Hz, 1H), 8.50 (d, J = 7.33 Hz, 1H), 8.40 (br s, 1H), 8.20 (d, J = 2.02 Hz, 1H), 8.08-8.12 (m, 1H), 8.03 (d, J = 8.08 Hz, 1H), 7.43-7.54 (m, 3H), 7.29-7.36 (m, 1H), 7.05-7.11 (m, 1H), 4.23-4.32 (m, 1H), 2.89-3.02 (m, 2H), 2.70-2.83 (m, 2H) | 513.52 | D | |
| I- 642 | 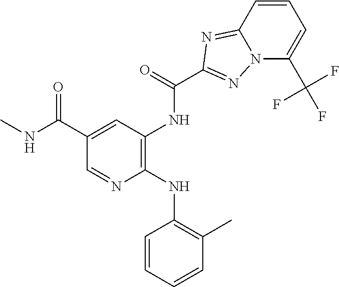 [SMILES: CNC(=O)c1cnc(Nc2ccccc2C)c(NC(=O)c2nc3cccc(C(F)(F)F)n3n2)c1] | | | D | |
| I- 643 | 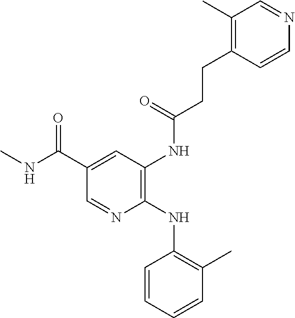 [SMILES: CNC(=O)c1cnc(Nc2ccccc2C)c(NC(=O)CCc2ccncc2C)c1] | | | D | |
| I- 644 | 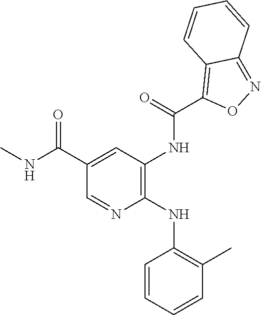 [SMILES: CNC(=O)c1cnc(Nc2ccccc2C)c(NC(=O)c2onc3ccccc23)c1] | | | D | |
| I- 645 | 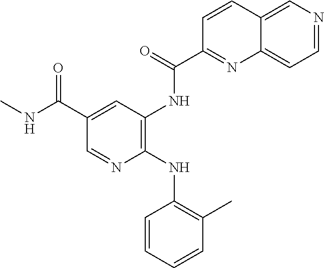 [SMILES: CNC(=O)c1cnc(Nc2ccccc2C)c(NC(=O)c2ccc3cnccc3n2)c1] | | | D | |
| I- 646 | 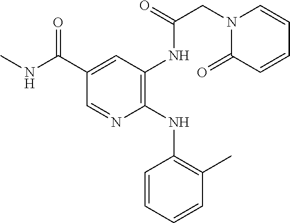 [SMILES: CNC(=O)c1cnc(Nc2ccccc2C)c(NC(=O)Cn2ccccc2=O)c1] | | | D | |
| I- 647 | 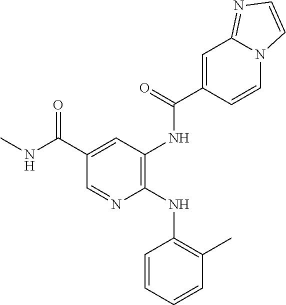 [SMILES: CNC(=O)c1cnc(Nc2ccccc2C)c(NC(=O)c2ccn3ccnc3c2)c1] | | | D | |
| I- 648 | 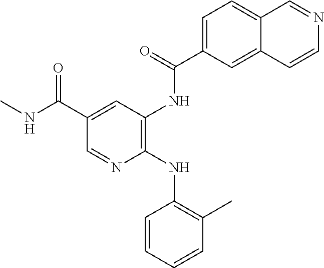 [SMILES: CNC(=O)c1cnc(Nc2ccccc2C)c(NC(=O)c2ccc3cnccc3c2)c1] | | | D | |
| I- 649 | 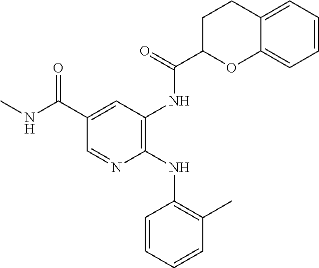 [SMILES: CNC(=O)c1cnc(Nc2ccccc2C)c(NC(=O)C2CCc3ccccc3O2)c1] | | | D | |
| I- 650 | 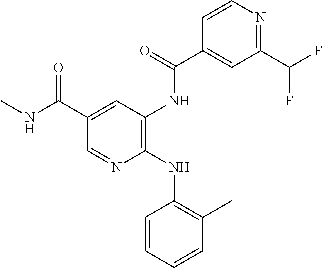 [SMILES: CNC(=O)c1cnc(Nc2ccccc2C)c(NC(=O)c2ccnc(C(F)F)c2)c1] | | | D | |
| I- 651 | 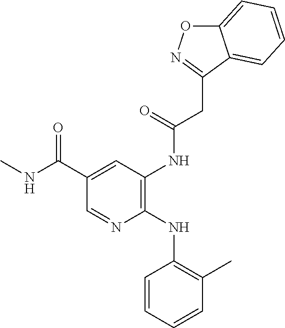 [SMILES: CNC(=O)c1cnc(Nc2ccccc2C)c(NC(=O)Cc2noc3ccccc23)c1] | | | D | |
| I- 652 | 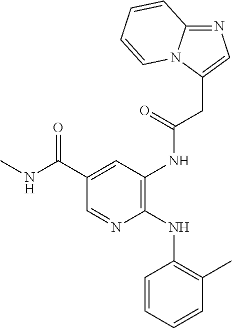 | | | B | D |
| I- 653 | 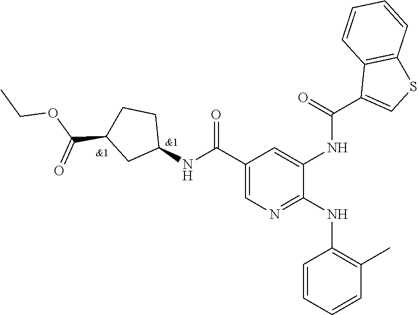 [SMILES: CCOC(=O)[C@H]1CC[C@@H](NC(=O)c2cnc(Nc3ccccc3C)c(NC(=O)c3csc4ccccc34)c2)C1] | | | C | |
| I- 654 | 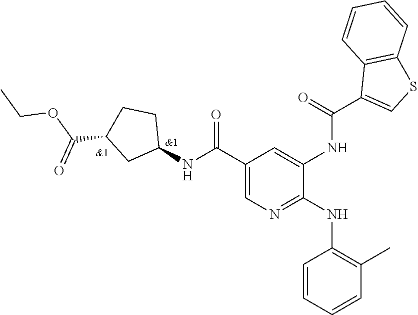 [SMILES: CCOC(=O)[C@@H]1CC[C@@H](NC(=O)c2cnc(Nc3ccccc3C)c(NC(=O)c3csc4ccccc34)c2)C1] | (400 MHz, cdcl3) 8.48 (d, J = 2.0 Hz, 1H), 8.43 (d, J = 7.4 Hz, 1H), 8.23 (d, J = 2.0 Hz, 1H), 7.99 (s, 1H), 7.89 (d, J = 7.5 Hz, 1H), 7.77 (s, 1H), 7.51-6.40 (m, 3H), 7.22 (d, J = 8.0 Hz, 1H), 7.17 (t, J = 8.0 Hz, 1H), 7.09-6.99 (m, 2H), 6.05 (d, J = 7.4 Hz, 1H), 4.48 (dd, J = 13.5, 6.5 Hz, 1H), 4.13 (q, J = 7.2 Hz, 2H), 3.00-2.87 (m, 1H), 2.34 (dd, J = 14.2, 7.0 Hz, 1H), 2.30 (s, 3H), 2.24 (t, J = 9.8 Hz, 1H), 2.12-1.99 (m, 1H), 1.94-1.81 (m, 3H), 1.25 (t, J = 7.1 Hz, 3H). | 543 | D | |
| I- 655 | 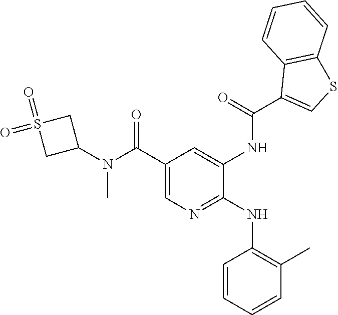 [SMILES: Cc1ccccc1Nc1ncc(C(=O)N(C)C2CS(=O)(=O)C2)cc1NC(=O)c1csc2ccccc12] | (400 MHz, cdcl3) 8.57 (d, J = 2.2 Hz, 1H), 8.49 (d, J = 7.5 Hz, 1H), 8.34 (s, 1H), 8.28 (d, J = 2.2 Hz, 1H), 7.99 (s, 1H), 7.89 (d, J = 7.5 Hz, 1H), 7.57-7.40 (m, 4H), 7.35 (s, 1H), 7.22 (d, J = 7.9 Hz, 1H), 7.18 (td, J = 7.4, 1.2 Hz, 1H), 7.03 (td, J = 7.4, 1.2 Hz, 1H), 4.52 (m, 1H), 4.15 (q, J = 7.1 Hz, 2H), 2.94 (m, 1H), 2.33 (s, 3H), 2.14-2.03 (m, 2H), 1.94-1.82 (m, 4H), 1.26 (t, J = 7.1 Hz, 3H). | 543 | B | D |
| I- 656 | 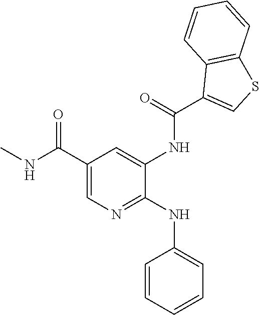 [SMILES: CNC(=O)c1cnc(Nc2ccccc2)c(NC(=O)c2csc3ccccc23)c1] | (400 MHz, dmso) 10.02 (s, 1H), 8.68 (s, 1H), 8.44 (d, J = 7.4 Hz, 1H), 8.22 (s, 1H), 8.07 (d, J = 7.4 Hz, 1H), 8.04 (d, J = 1.9 Hz, 1H), 7.79 (d, J = 1.9 Hz, 1H), 7.49-7.39 (m, 3H), 7.21 (d, J = 7.4 Hz, 1H), 7.16 (t, J = 7.4 Hz, 1H), 7.04 (t, J = 7.4 Hz, 1H), 5.10-4.94 (m, 1H), 4.64-4.45 (m, 4H), 3.08 (s, 3H), 2.16 (s, 3H). | 520.9 | B | E |
| I- 657 | 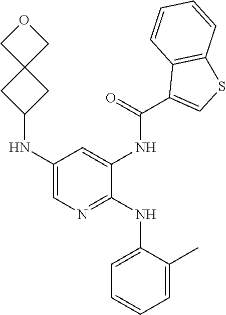 [SMILES: Cc1ccccc1Nc1ncc(NC2CC3(COC3)C2)cc1NC(=O)c1csc2ccccc12] | (400 MHz, dmso) 9.92 (s, 1H), 8.72 (s, 1H), 8.66 (s, 1H), 8.54 (d, J = 2.2 Hz, 1H), 8.51-8.46 (m, 1H), 8.38 (q, J = 4.5 Hz, 1H), 8.16 (d, J = 2.2 Hz, 1H), 8.13- 8.05 (m, 1H), 7.71-7.60 (m, 2H), 7.54- 7.41 (m, 2H), 7.36-7.23 (m, 2H), 7.05- 6.92 (m, 1H), 2.79 (d, J = 4.5 Hz, 3H). | 403 | D | |
| I- 658 | 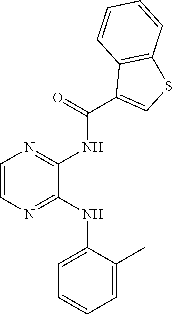 [SMILES: Cc1ccccc1Nc1nccnc1NC(=O)c1csc2ccccc12] | (400 MHz, dmso) 10.18 (s, 1H), 8.59 (s, 1H), 8.46 (dd, J = 7.1, 1.4 Hz, 1H), 8.09 (dd, J = 6.9, 1.6 Hz, 1H), 7.73 (d, J = 2.7 Hz, 1H), 7.56-7.43 (m, 4H), 7.32 (d, J = 2.6 Hz, 1H), 7.12 (s, 1H), 7.08 (d, J = 7.7 Hz, 1H), 7.06-7.00 (m, 1H), 6.75 (td, J = 7.4, 1.0 Hz, 1H), 4.73-4.65 (m, 1H), 4.34 (s, 1H), 3.64 (d, J = 3.2 Hz, 2H), 2.15 (s, 3H), 1.79- 1.74 (m, 2H), 1.39 (dd, J = 4.1, 1.4 Hz, 2H). | 471.1 | D | |
| I- 659 | 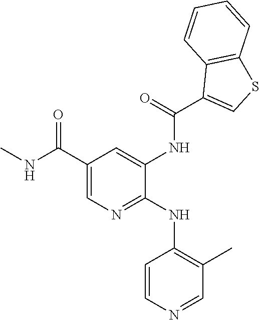 [SMILES: CNC(=O)c1cnc(Nc2ccncc2C)c(NC(=O)c2csc3ccccc23)c1] | (400 MHz, DMSO) 10.89 (s, 1H), 8.76 (s, 1H), 8.53 (d, J = 7.5 Hz, 1H), 8.19 (s, 1H), 8.09 (dd, J = 7.2, 1.1 Hz, 1H), 7.99 (d, J = 2.6 Hz, 1H), 7.84 (d, J = 2.7 Hz, 1H), 7.55-7.41 (m, 3H), 7.25- 7.14 (m, 2H), 7.04 (m, 1H), 2.19 (s, 3H). | 361 | D | |
| I- 660 | 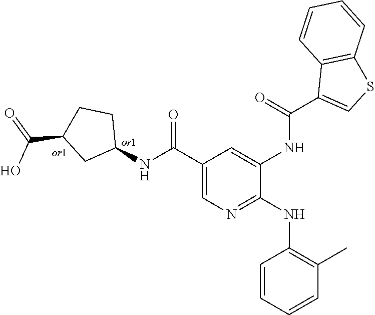 [SMILES: Cc1ccccc1Nc1ncc(C(=O)N[C@@H]2CC[C@H](C(=O)O)C2)cc1NC(=O)c1csc2ccccc12] | (400 MHz, dmso) 10.45 (s, 1H), 8.75- 8.41 (m, 5H), 8.33 (s, 1H), 8.26 (s, 1H), 8.25 (m, 1H), 8.09 (d, J = 7.9 Hz, 1H), 7.96 (s, 1H), 7.54-7.41 (m, 2H), 2.80 (d, J = 4.5 Hz, 3H), 2.20 (s, 3H). | 416.1 | D | |
| I- 661 | 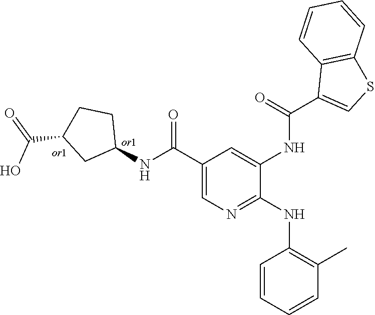 [SMILES: Cc1ccccc1Nc1ncc(C(=O)N[C@@H]2CC[C@@H](C(=O)O)C2)cc1NC(=O)c1csc2ccccc12] | (400 MHz, dmso) 12.05 (s, 1H), 10.05 (s, 1H), 8.68 (s, 1H), 8.47 (dd, J = 7.1, 1.3 Hz, 1H), 8.43 (d, J = 2.2 Hz, 1H), 8.21-8.15 (m, 2H), 8.10-8.05 (m, 2H), 7.51-7.40 (m, 3H), 7.19 (d, J = 7.6 Hz, 1H), 7.15 (td, J = 7.6, 1.3 Hz, 1H), 7.03 (td, J = 7.6, 1.3 Hz, 1H), 4.30 (dd, J = 13.4, 6.5 Hz, 1H), 2.88 (dt, J = 15.3, 7.7 Hz, 1H), 2.15 (s, 3H), 2.07-2.00 (m, 1H), 1.99-1.92 (m, 2H), 1.82-1.65 (m, 2H), 1.57 (ddd, J = 13.3, 8.3, 5.3 Hz, 1H). | 515.3 | D | |
| I- 662 | 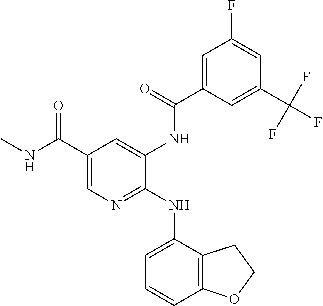 [SMILES: CNC(=O)c1cnc(Nc2cccc3c2CCO3)c(NC(=O)c2cc(F)cc(C(F)(F)F)c2)c1] | (400 MHz, dmso) 12.08 (s, 1H), 10.04 (s, 1H), 8.68 (s, 1H), 8.47 (d, J = 7.1 Hz, 1H), 8.43 (d, J = 2.1 Hz, 1H), 8.24 (d, J = 7.4 Hz, 1H), 8.17 (s, 1H), 8.10 (d, J = 2.1 Hz, 1H), 8.07 (dd, J = 7.1, 1.1 Hz, 1H), 7.51-7.40 (m, 3H), 7.20 (d, J = 7.4 Hz, 1H), 7.15 (td, J = 7.4, 1.2 Hz, 1H), 7.03 (td, J = 7.4, 1.2 Hz, 1H), 4.37-4.16 (m, 1H), 2.80-2.67 (m, 1H), 2.20-2.11 (m, 4H), 1.95-1.67 (m, 4H), 1.65-1.51 (m, 1H). | 515.3 | C | |
| I- 663 | 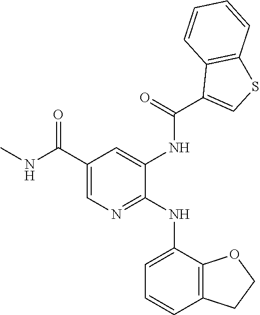 [SMILES: CNC(=O)c1cnc(Nc2cccc3c2OCC3)c(NC(=O)c2csc3ccccc23)c1] | (400 MHz, DMSO) 10.32 (s, 1H), 8.50 (d, J = 2.1 Hz, 1H), 8.41 (s, 1H), 8.35- 8.29 (m, 1H), 8.21 (s, 1H), 8.14 (d, J = 9.0 Hz, 1H), 8.04 (d, J = 2.0 Hz, 1H), 7.97 (d, J = 8.4 Hz, 1H), 7.02 (t, J = 7.9 Hz, 1H), 6.93 (d, J = 8.0 Hz, 1H), 6.50 (d, J = 7.8 Hz, 1H), 4.46 (t, J = 8.7 Hz, 2H), 2.98 (t, J = 8.7 Hz, 2H), 2.75 (d, J = 4.4 Hz, 3H). | 475.3 | D | |
| I- 664 | 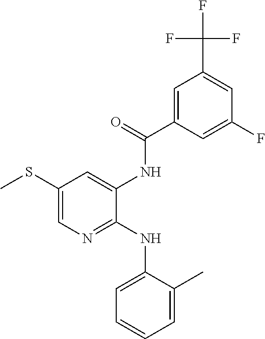 [SMILES: CSc1cnc(Nc2ccccc2C)c(NC(=O)c2cc(F)cc(C(F)(F)F)c2)c1] | (400 MHz, DMSO) 10.21 (s, 1H), 8.69 (s, 1H), 8.53 (d, J = 2.2 Hz, 1H), 8.47 (dd, J = 7.1, 1.1 Hz, 1H), 8.38 (q, J = 4.2 Hz, 1H), 8.13 (d, J = 2.2 Hz, 1H), 8.11 (dd, J = 7.2, 1.1 Hz, 1H), 8.03 (s, 1H), 7.67 (d, J = 7.5 Hz, 1H), 7.49 (dqd, J = 14.7, 7.2, 1.3 Hz, 2H), 6.94 (dd, J = 7.3, 0.9 Hz, 1H), 6.84-6.77 (m, 1H), 4.51 (t, J = 8.7 Hz, 2H), 3.21 (t, J = 8.7 Hz, 2H), 2.79 (d, J = 4.5 Hz, 3H). | 445.2 | D | |
| I- 665 | 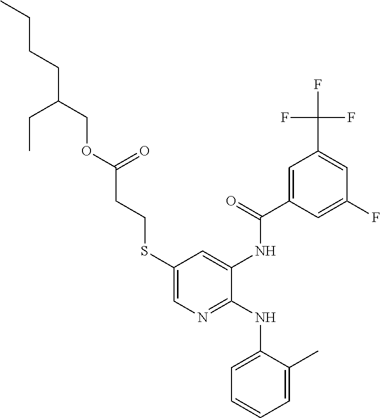 [SMILES: CCCCC(CC)COC(=O)CCSc1cnc(Nc2ccccc2C)c(NC(=O)c2cc(F)cc(C(F)(F)F)c2)c1] | (400 MHz, cdcl3) 8.44 (d, J = 2.2 Hz, 1H), 8.13 (d, J = 2.2 Hz, 1H), 7.60 (s, 1H), 7.46-7.43 (m, 1H), 7.31-7.25 (m, 3H + CDCl3), 7.10 (td, J = 7.6, 1.3 Hz, 1H), 7.00 (td, J = 7.5, 1.2 Hz, 1H), 6.86 (dd, J = 7.9, 0.9 Hz, 1H), 6.20 (s, 1H), 2.54 (s, 3H), 2.35 (s, 3H). | 436.3 | D | |
| I- 666 | 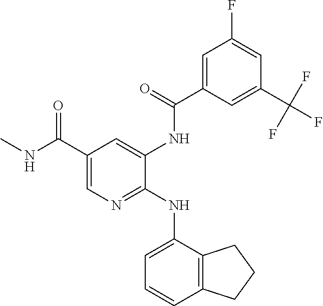 [SMILES: CNC(=O)c1cnc(Nc2cccc3c2CCC3)c(NC(=O)c2cc(F)cc(C(F)(F)F)c2)c1] | (400 MHz, cd3od) 8.15 (s, 1H), 8.01 (d, J = 2.2 Hz, 1H), 8.01-7.93 (m, 1H), 7.79 (d, J = 2.2 Hz, 1H), 7.75-7.69 (m, 1H), 7.30 (dd, J = 7.9, 1.3 Hz, 1H), 7.23-7.19 (m, 1H), 7.14 (m, 1H), 7.04 (td, J = 7.4, 1.3 Hz, 1H), 4.01 (d, J = 5.6 Hz, 2H), 3.02 (t, J = 6.8 Hz, 2H), 2.60 (t, J = 6.8 Hz, 2H), 2.22 (s, 3H), 1.55 (dq, J = 12.1, 6.0 Hz, 1H), 1.43- 1.33 (m, 2H), 1.33-1.24 (m, 6H), 0.91- 0.86 (m, 6H). | 606.7 | B | C |
| I- 667 | 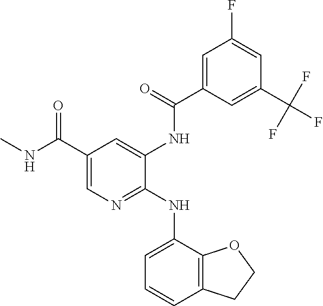 [SMILES: CNC(=O)c1cnc(Nc2cccc3c2OCC3)c(NC(=O)c2cc(F)cc(C(F)(F)F)c2)c1] | (400 MHz, DMSO) 10.21 (s, 1H), 8.48 (d, J = 2.2 Hz, 1H), 8.35-8.26 (m, 2H), 8.23 (s, 1H), 8.16 (d, J = 9.1 Hz, 1H), 8.04 (d, J = 2.2 Hz, 1H), 7.99 (d, J = 8.5 Hz, 1H), 7.26 (d, J = 7.8 Hz, 1H), 7.10 (t, J = 7.6 Hz, 1H), 6.99 (d, J = 7.3 Hz, 1H), 2.88 (t, J = 7.4 Hz, 2H), 2.77 (d, J = 4.5 Hz, 3H), 2.70 (t, J = 7.3 Hz, 2H), 1.95 (p, J = 7.5 Hz, 2H). | 473 | D | |
| I- 668 | 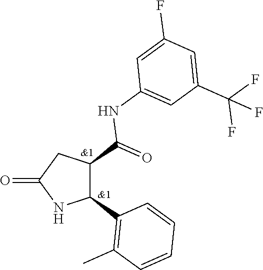 [SMILES: Cc1ccccc1[C@H]1NC(=O)C[C@H]1C(=O)Nc1cc(F)cc(C(F)(F)F)c1] | (400 MHz, DMSO) 10.30 (s, 1H), 8.46 (d, J = 2.0 Hz, 1H), 8.31 (q, J = 4.0 Hz, 1H), 8.24 (s, 2H), 8.17 (d, J = 9.1 Hz, 1H), 8.10 (s, 1H), 7.98 (d, J = 8.5 Hz, 1H), 7.47 (d, J = 6.9 Hz, 1H), 6.97 (d, J = 6.5 Hz, 1H), 6.83-6.77 (m, 1H), 4.50 (t, J = 8.7 Hz, 2H), 3.21 (t, J = 8.7 Hz, 2H), 2.77 (d, J = 4.5 Hz, 3H). | 475.3 | D | |
| I- 669 | 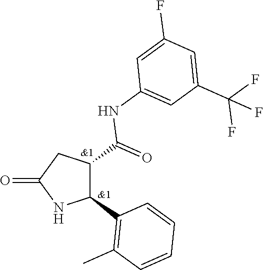 [SMILES: Cc1ccccc1[C@H]1NC(=O)C[C@@H]1C(=O)Nc1cc(F)cc(C(F)(F)F)c1] | (400 MHz, dmso) 10.22 (s, 1H), 8.04 (s, 1H), 7.22 (m, 3H), 7.18 (d, J = 7.7 Hz, 1H), 7.11-7.03 (m, 1H), 7.00 (m, 2H), 5.29 (d, J = 8.3 Hz, 1H), 3.70 (td, J = 8.5, 5.3 Hz, 1H), 2.61 (dd, J = 16.6, 5.2 Hz, 1H), 2.56-2.44 (m, 4H)+residual dmso, 2.33 (s, 3H). | 381 | D | |
| I- 670 | 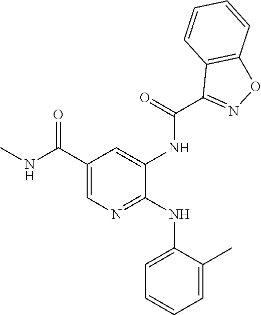 [SMILES: CNC(=O)c1cnc(Nc2ccccc2C)c(NC(=O)c2noc3ccccc23)c1] | (400 MHz, dmso) 10.68 (s, 1H), 8.17 (s, 1H), 7.79 (s, 1H), 7.78-7.73 (m, 1H), 7.38 (d, J = 8.5 Hz, 1H), 7.35-7.31 (m, 1H), 7.26 (td, J = 7.5, 1.9 Hz, 1H), 7.23-7.15 (m, 2H), 5.10 (d, J = 5.4 Hz, 1H), 3.08 (ddd, J = 9.6, 6.9, 5.4 Hz, 1H), 2.69 (dd, J = 16.8, 9.6 Hz, 1H), 2.44 (dd, J = 16.9, 6.9 Hz, 1H), 2.24 (s, 3H). | 381 | D | |
| I- 671 | 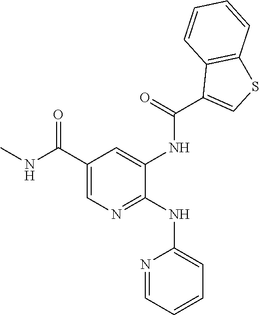 [SMILES: CNC(=O)c1cnc(Nc2ccccn2)c(NC(=O)c2csc3ccccc23)c1] | (400 MHz, dmso) 10.65 (s, 1H), 8.44 (d, J = 2.2 Hz, 1H), 8.31 (m, 2H), 8.18 (dt, J = 8.0, 1.0 Hz, 1H), 8.06 (d, J = 2.2 Hz, 1H), 7.92 (d, J = 8.5 Hz, 1H), 7.77 (ddd, J = 8.5, 7.1, 1.2 Hz, IH), 7.54 (ddd, J = 7.9, 7.2, 0.7 Hz, 1H), 7.34 (d, J = 7.7 Hz, 1H), 7.22 (d, J = 7.4 Hz, 1H), 7.17 (td, J = 7.5, 1.2 Hz, 1H), 7.07 (td, J = 7.4, 1.3 Hz, 1H), 2.77 (d, J = 4.5 Hz, 3H), 2.17 (s, 3H). | 402.3 | D | |
| I- 672 | 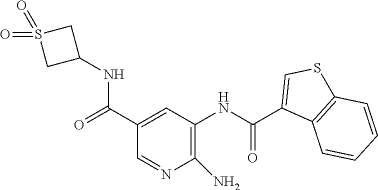 [SMILES: Nc1ncc(C(=O)NC2CS(=O)(=O)C2)cc1NC(=O)c1csc2ccccc12] | (400 MHz, dmso) 10.32 (br s, 1H), 9.15 (s, 1H), 8.69 (s, 1H), 8.64 (d, J = 2.2 Hz, 1H), 8.50 (q, J = 4.0 Hz, 1H), 8.47-8.40 (m, 1H), 8.31 (d, J = 2.2 Hz, 1H), 8.26 (s, 1H), 8.25-8.21 (m, 1H), 8.14-8.03 (m, 2H), 7.81-7.70 (m, 1H), 7.55-7.37 (m, 2H), 6.97 (ddd, J = 7.3, 4.9, 0.9 Hz, 1H), 2.81 (d, J = 4.5 Hz, 3H). Obtained as a formate salt. | 404.2 | D | |
| I- 673 | 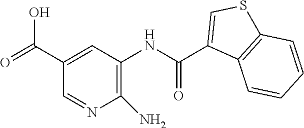 [SMILES: Nc1ncc(C(=O)O)cc1NC(=O)c1csc2ccccc12] | | | D | |
| I- 674 | 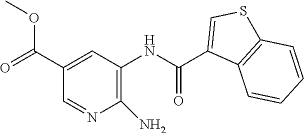 [SMILES: COC(=O)c1cnc(N)c(NC(=O)c2csc3ccccc23)c1] | | | D | |
| I- 675 | 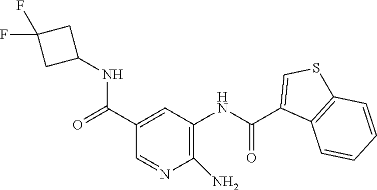 [SMILES: Nc1ncc(C(=O)NC2CC(F)(F)C2)cc1NC(=O)c1csc2ccccc12] | | | D | |
| I- 676 | 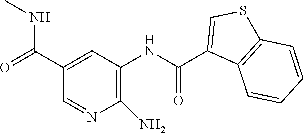 [SMILES: CNC(=O)c1cnc(N)c(NC(=O)c2csc3ccccc23)c1] | | | C | D |
| I- 677 | 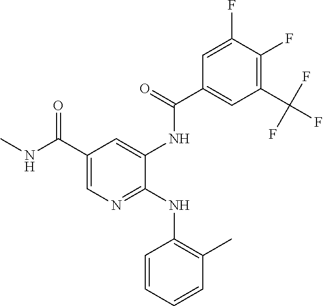 [SMILES: CNC(=O)c1cnc(Nc2ccccc2C)c(NC(=O)c2cc(F)c(F)c(C(F)(F)F)c2)c1] | | | D | D |
| I- 678 | 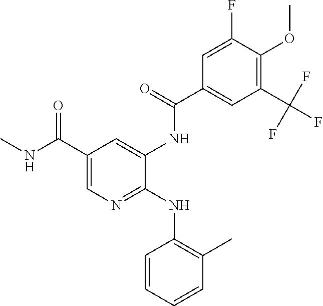 [SMILES: CNC(=O)c1cnc(Nc2ccccc2C)c(NC(=O)c2cc(F)c(OC)c(C(F)(F)F)c2)c1] | | 465.2 | D | D |
| I- 679 | 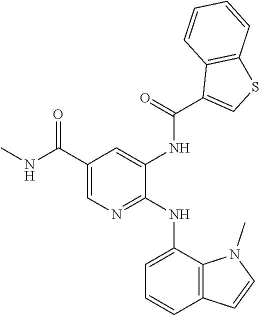 [SMILES: CNC(=O)c1cnc(Nc2cccc3ccn(C)c23)c(NC(=O)c2csc3ccccc23)c1] | | 477.2 | D | D |
| I- 680 | 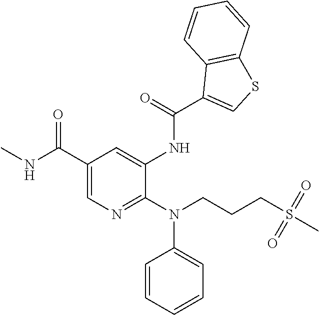 | (400 MHz, DMSO) 10.02 (s, 1H), 8.74 (s, 1H), 8.72 (s, 1H), 8.49 (dd, J = 7.1, 1.1 Hz, 1H), 8.32 (d, J = 2.2 Hz, 1H), 8.28 (q, J = 4.3 Hz, 1H), 8.09 (m, 2H), 7.52-7.40 (m, 3H), 7.17 (d, J = 3.1 Hz, 1H), 6.99 (t, J = 7.6 Hz, 1H), 6.90 (d, J = 7.1 Hz, 1H), 6.41 (d, J = 3.1 Hz, 1H), 3.73 (s, 3H), 2.76 (d, J = 4.5 Hz, 3H). | 456.3 | D | D |
| I- 681 | 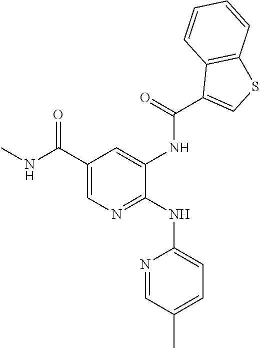 [SMILES: CNC(=O)c1cnc(Nc2ccc(C)cn2)c(NC(=O)c2csc3ccccc23)c1] | (400 MHz, dmso) 9.16 (s, 1H), 8.44 (s, 1H), 8.24-8.14 (m, 1H), 8.12 (d, J = 7.9 Hz, 1H), 7.92 (d, J = 8.1 Hz, 1H), 7.78- 7.64 (m, 3H), 7.56 (s, 1H), 7.43 (t, J = 7.7 Hz, 1H), 7.40-7.35 (m, 1H), 7.35- 7.27 (m, 2H), 7.05 (t, J = 7.4 Hz, 1H), 4.46-4.22 (m, 1H), 3.31-3.13 (m, 3H), 2.99 (s, 3H), 2.65 (d, J = 3.9 Hz, 3H), 2.27-2.13 (m, 1H), 2.03-1.78 (m, 1H). | 523.3 | D | |
| I- 682 | 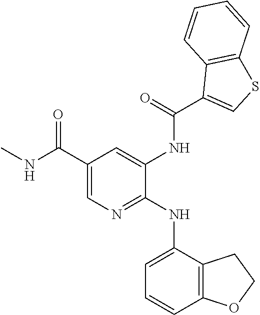 [SMILES: CNC(=O)c1cnc(Nc2cccc3c2CCO3)c(NC(=O)c2csc3ccccc23)c1] | (400 MHz, dmso) 10.32 (br s, 1H), 9.03 (s, 1H), 8.71 (d, J = 1.9 Hz, 1H), 8.62 (d, J = 2.1 Hz, 1H), 8.52-8.46 (m, 1H), 8.46-8.40 (m, 1H), 8.33 (s, 1H), 8.27 (d, J = 2.0 Hz, 1H), 8.17-8.05 (m, 2H), 8.03 (d, J = 8.5 Hz, 1H), 7.58 (dd, J = 7.7, 1.4 Hz, 1H), 7.54-7.37 (m, 2H), 2.80 (d, J = 4.2 Hz, 3H), 2.22 (s, 3H). | 418.3 | C | |
| I- 683 | 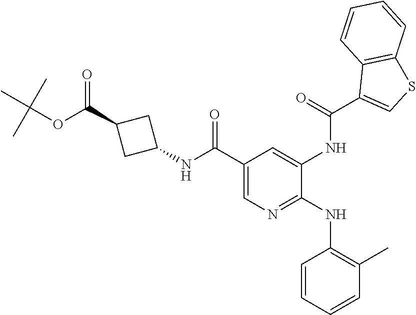 [SMILES: Cc1ccccc1Nc1ncc(C(=O)N[C@H]2C[C@H](C(=O)OC(C)(C)C)C2)cc1NC(=O)c1csc2ccccc12] | (400 MHz, DMSO) 10.12 (s, 1H), 8.71 (s, 1H), 8.55 (d, J = 2.0 Hz, 1H), 8.49 (dd, J = 7.1, 1.2 Hz, 1H), 8.39 (dd, J = 8.9, 4.3 Hz, 1H), 8.36 (s, 1H), 8.17 (d, J = 2.2 Hz, 1H), 8.10 (dd, J = 7.1, 1.2 Hz, 1H), 7.53-7.43 (m, 2H), 7.09-7.01 (m, 2H), 6.54-6.46 (m, 1H), 4.49 (t, J = 8.7 Hz, 2H), 3.03 (t, J = 8.7 Hz, 2H), 2.78 (d, J = 4.5 Hz, 3H). | 445.3 | C | E |
| I- 684 | 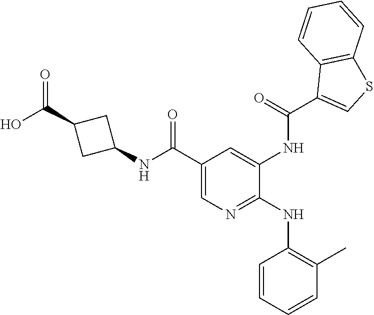 [SMILES: Cc1ccccc1Nc1ncc(C(=O)N[C@H]2C[C@@H](C(=O)O)C2)cc1NC(=O)c1csc2ccccc12] | (400 MHz, dmso) 10.03 (s, 1H), 8.68 (s, 1H), 8.55 (d, J = 7.4 Hz, 1H), 8.48 (dd, J = 7.4, 1.2 Hz, 1H), 8.43 (d, J = 2.2 Hz, 1H), 8.21 (s, 1H), 8.13-8.02 (m, 2H), 7.51-7.38 (m, 3H), 7.20 (d, J = 7.4 Hz, 1H), 7.15 (td, J = 7.4, 1.2 Hz, 1H), 7.03 (td, J = 7.4, 1.2 Hz, 1H), 4.57-4.47 (m, 1H), 2.98-2.82 (m, 1H), 2.43-2.34 (m, 2H), 2.34-2.22 (m, 2H), 2.15 (s, 3H), 1.41 (s, 9H). | 557.4 | C | D |
| I- 685 | 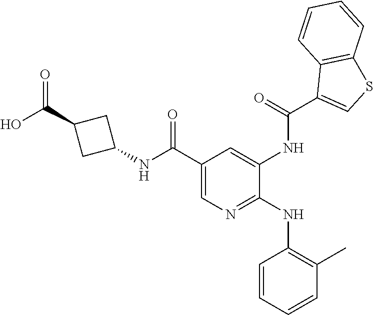 [SMILES: Cc1ccccc1Nc1ncc(C(=O)N[C@H]2C[C@H](C(=O)O)C2)cc1NC(=O)c1csc2ccccc12] | (400 MHz, dmso) 10.10 (s, 1H), 8.70 (s, 1H), 8.54 (d, J = 7.8 Hz, 1H), 8.48 (d, J = 7.8 Hz, 1H), 8.43 (d, J = 2.2 Hz, 1H), 8.23 (s, 1H), 8.13 (d, J = 2.2 Hz, 1H), 8.07 (d, J = 7.5 Hz, 1H), 7.51-7.39 (m, 3H), 7.20 (d, J = 7.5 Hz, 1H), 7.15 (td, J = 7.5, 1.2 Hz, 1H), 7.03 (td, J = 7.5, 1.2 Hz, 1H), 4.41-4.26 (m, 1H), 2.73-2.66 (m, 1H), 2.43-2.36 (m, 2H), 2.24-2.12 (m, 5H). | 501.3 | C | D |
| I- 686 | 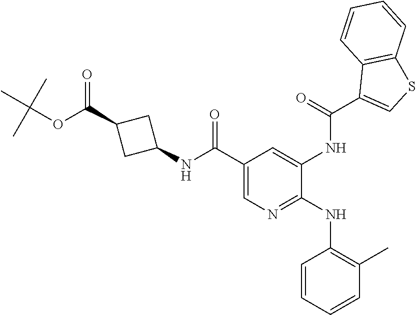 [SMILES: Cc1ccccc1Nc1ncc(C(=O)N[C@H]2C[C@@H](C(=O)OC(C)(C)C)C2)cc1NC(=O)c1csc2ccccc12] | (400 MHz, dmso) 12.21 (s, 1H), 10.04 (s, 1H), 8.68 (s, 1H), 8.55 (d, J = 7.4 Hz, 1H), 8.47 (d, J = 7.4 Hz, 1H), 8.43 (d, J = 2.2 Hz, 1H), 8.23 (s, 1H), 8.10 (d, J = 2.2 Hz, 1H), 8.07 (d, J = 7.4 Hz, 1H), 7.52-7.38 (m, 3H), 7.20 (d, J = 7.8 Hz, 1H), 7.16 (t, J = 7.8 Hz, 1H), 7.04 (t, J = 7.8 Hz, 1H), 4.60-4.47 (m, 1H), 2.98-2.89 (m, 1H), 2.43-2.39 (m, 2H), 2.35-2.25 (m, 2H), 2.15 (s, 3H). | 501.3 | D | D |
| I- 687 | 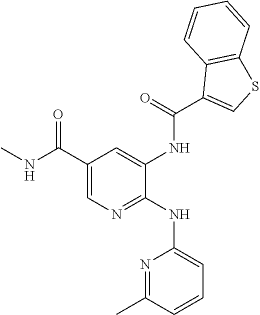 [SMILES: CNC(=O)c1cnc(Nc2cccc(C)n2)c(NC(=O)c2csc3ccccc23)c1] | (400 MHz, dmso) 10.03 (s, 1H), 8.69 (s, 1H), 8.54 (d, J = 7.4 Hz, 1H), 8.49 (dd, J = 7.4, 1.1 Hz, 1H), 8.45 (d, J = 2.2 Hz, 1H), 8.21 (s, 1H), 8.13 (d, J = 2.2 Hz, 1H), 8.07 (dd, J = 7.4, 1.1 Hz, 1H), 7.51-7.39 (m, 3H), 7.20 (d, J = 7.4 Hz, 1H), 7.16 (t, J = 7.4 Hz, 1H), 7.03 (td, J = 7.4, 1.1 Hz, 1H), 4.44-4.28 (m, 1H), 2.79-2.66 (m, 1H), 2.43-2.36 (m, 2H), 2.23-2.11 (m, 5H), 1.38 (s, 9H). | 557.4 | D | |
| I- 688 | 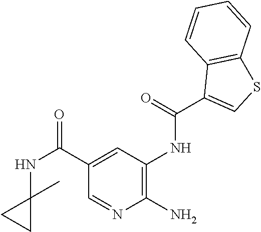 [SMILES: CC1(NC(=O)c2cnc(N)c(NC(=O)c3csc4ccccc34)c2)CC1] | (400 MHz, dmso) 10.24 (s, 1H), 8.98 (s, 1H), 8.64 (s, 1H), 8.61 (d, J = 2.2 Hz, 1H), 8.46 (q, J = 4.6 Hz, 1H), 8.43- 8.38 (m, 1H), 8.26 (d, J = 2.2 Hz, 1H), 8.13-8.03 (m, 1H), 7.92 (d, J = 8.3 Hz, 1H), 7.65-7.55 (m, 1H), 7.52-7.36 (m, 2H), 6.81 (d, J = 7.3 Hz, 1H), 2.78 (d, J = 4.5 Hz, 3H), 2.31 (s, 3H). | 418.3 | D | |
| I- 689 | 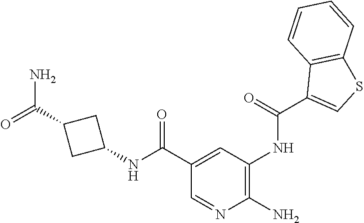 [SMILES: Nc1ncc(C(=O)N[C@H]2C[C@@H](C(N)=O)C2)cc1NC(=O)c1csc2ccccc12] | | | D | |
| I- 690 | 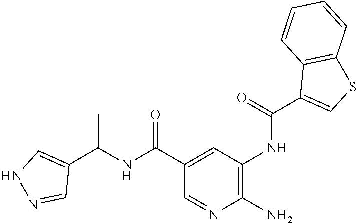 [SMILES: CC(NC(=O)c1cnc(N)c(NC(=O)c2csc3ccccc23)c1)c1cn[nH]c1] | | | D | |
| I- 691 | 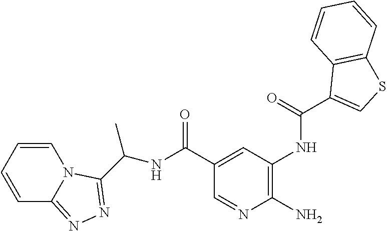 [SMILES: CC(NC(=O)c1cnc(N)c(NC(=O)c2csc3ccccc23)c1)c1nnc2ccccn12] | | | B | C |
| I- 692 | 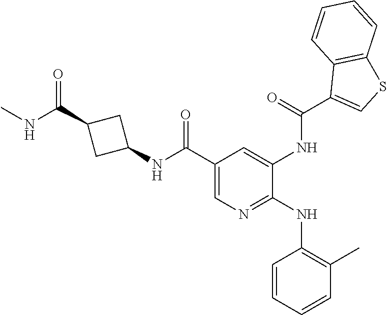 [SMILES: CNC(=O)[C@H]1C[C@@H](NC(=O)c2cnc(Nc3ccccc3C)c(NC(=O)c3csc4ccccc34)c2)C1] | | | B | D |
| I- 693 | 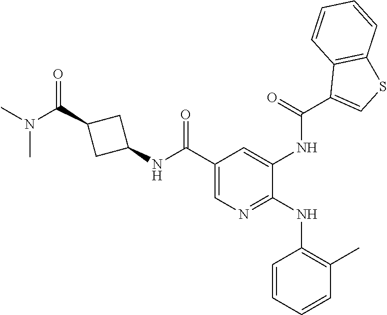 [SMILES: Cc1ccccc1Nc1ncc(C(=O)N[C@H]2C[C@@H](C(=O)N(C)C)C2)cc1NC(=O)c1csc2ccccc12] | (400 MHz, dmso) 10.13 (s, 1H), 8.71 (s, 1H), 8.56 (d, J = 7.6 Hz, 1H), 8.48 (d, J = 7.6 Hz, 1H), 8.43 (d, J = 2.2 Hz, 1H), 8.25 (s, 1H), 8.15 (d, J = 2.2 Hz, 1H), 8.07 (d, J = 7.6 Hz, 1H), 7.70 (d, J = 4.5 Hz, 1H), 7.53-7.37 (m, 3H), 7.19 (d, J = 7.6 Hz, 1H), 7.15 (t, J = 7.6 Hz, 1H), 7.03 (t, J = 7.6 Hz, 1H), 4.43-4.22 (m, 1H), 2.68-2.56 (m, 1H), 2.54 (d, J = 4.5 Hz, 3H), 2.33-2.26 (m, 2H), 2.22-2.12 (m, 5H). | 514.3 | D | |
| I- 694 | 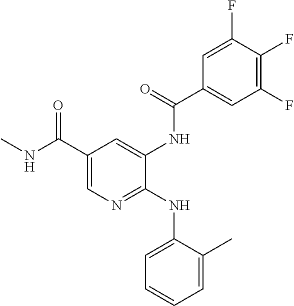 [SMILES: CNC(=O)c1cnc(Nc2ccccc2C)c(NC(=O)c2cc(F)c(F)c(F)c2)c1] | (400 MHz, dmso) 10.18 (s, 1H), 8.72 (s, 1H), 8.52 (d, J = 7.5 Hz, 1H), 8.48 (d, J = 7.5 Hz, 1H), 8.43 (d, J = 2.1 Hz, 1H), 8.27 (s, 1H), 8.14 (d, J = 2.2 Hz, 1H), 8.07 (d, J = 7.5 Hz, 1H), 7.51-7.36 (m, 3H), 7.19 (d, J = 7.5 Hz, 1H), 7.15 (t, J = 7.5 Hz, 1H), 7.03 (t, J = 7.5 Hz, 1H), 4.43-4.27 (m, 1H), 3.10-2.94 (m, 1H), 2.89 (s, 3H), 2.78 (s, 3H), 2.43- 2.36 (m, 2H), 2.23-2.11 (m, 5H). | 528.3 | D | |
| I- 695 | 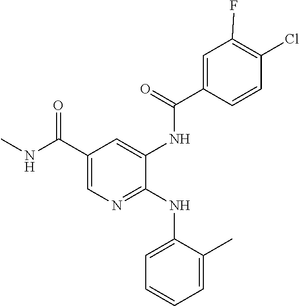 [SMILES: CNC(=O)c1cnc(Nc2ccccc2C)c(NC(=O)c2ccc(Cl)c(F)c2)c1] | (500 MHz, DMSO-d6) 10.13 (s, 1H), 8.41 (d, J = 2.2 Hz, 1H), 8.27 (q, J = 4.6 Hz, 1H), 8.25 (s, 1H), 8.01-7.96 (m, 3H), 7.32 (d, J = 7.3 Hz, 1H), 7.23 (d, J = 7.4 Hz, 1H), 7.18 (td, J = 7.5, 1.2 Hz, 1H), 7.09 (td, J = 7.4, 1.2 Hz, 1H), 2.76 (d, J = 4.5 Hz, 3H), 2.14 (s, 3H). | 415.2 | D | |
| I- 696 | 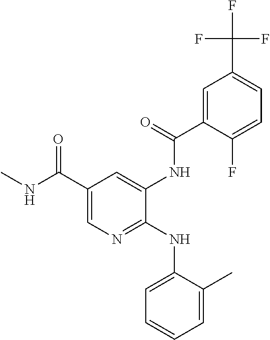 [SMILES: CNC(=O)c1cnc(Nc2ccccc2C)c(NC(=O)c2cc(C(F)(F)F)ccc2F)c1] | (500 MHz, DMSO-d6) 10.12 (s, 1H), 8.42 (d, J = 2.2 Hz, 1H), 8.27 (q, J = 4.3 Hz, 1H), 8.21 (s, 1H), 8.06 (dd, J = 10.2, 1.9 Hz, 1H), 8.01 (d, J = 2.2 Hz, 1H), 7.90 (dd, J = 8.4, 1.7 Hz, 1H), 7.80 (dd, J = 8.3, 7.6 Hz 1H), 7.35 (d, J = 7.3 Hz, 1H), 7.22 (d, J = 7.4 Hz, 1H), 7.18 (td, J = 7.6, 1.2 Hz, 1H), 7.07 (td, J = 7.4, 1.2 Hz, 1H), 2.76 (d, J = 4.5 Hz, 3H), 2.15 (s, 3H). | 413.2 | A | B |
| I- 697 | 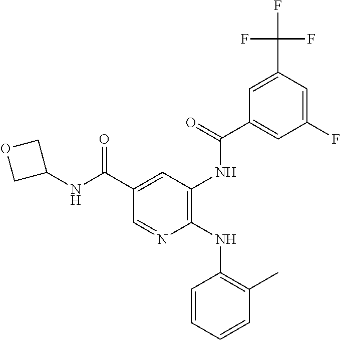 [SMILES: Cc1ccccc1Nc1ncc(C(=O)NC2COC2)cc1NC(=O)c1cc(F)cc(C(F)(F)F)c1] | (500 MHz, DMSO-d6) 10.01 (s, 1H), 8.43 (d, J = 2.1 Hz, 1H), 8.31 (q, J = 4.1 Hz, 1H), 8.24 (dd, J = 6.1, 2.0 Hz, 1H), 8.09 (d, J = 2.2 Hz, 1H), 8.07 (s, 1H), 8.03-7.98 (m, 1H), 7.64 (t, J = 9.2 Hz, 1H), 7.44 (d, J = 7.8 Hz, 1H), 7.24 (d, J = 7.5 Hz, 1H), 7.19 (td, J = 7.6, 1.1 Hz, 1H), 7.07 (td, J = 7.4, 1.1 Hz, 1H), 2.77 (d, J = 4.5 Hz, 3H), 2.18 (s, 3H). | 447.3 | B | B |
| I- 698 | 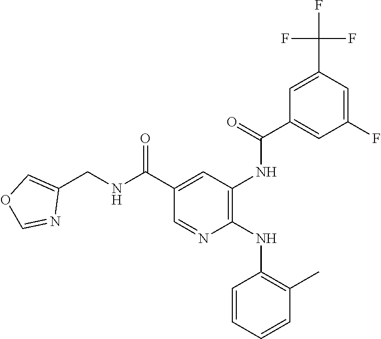 [SMILES: Cc1ccccc1Nc1ncc(C(=O)NCc2cocn2)cc1NC(=O)c1cc(F)cc(C(F)(F)F)c1] | (500 MHz, DMSO) 10.29 (s, 1H), 8.92 (d, J = 6.5 Hz, 1H), 8.47 (d, J = 2.1 Hz, 1H), 8.38 (s, 1H), 8.24 (s, 1H), 8.17 (d, J = 9.1 Hz, 1H), 8.06 (s, 1H), 7.98 (d, J = 8.3 Hz, 1H), 7.32 (s, 1H), 7.24 (d, J = 7.4 Hz, 1H), 7.18 (td, J = 7.6, 1.2 Hz, 1H), 7.09 (td, J = 7.4, 0.9 Hz, 1H), 5.04-4.94 (m, 1H), 4.76 (dd, J = 7.0, 6.5 Hz, 2H), 4.57 (t, J = 6.5 Hz, 2H), 2.16 (s, 3H). | 489.4 | A | B |
| I- 699 | 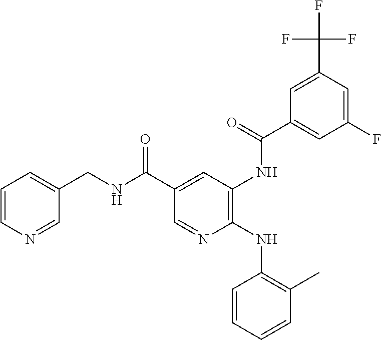 [SMILES: Cc1ccccc1Nc1ncc(C(=O)NCc2cccnc2)cc1NC(=O)c1cc(F)cc(C(F)(F)F)c1] | (500 MHz, DMSO) 10.28 (br. s, 1H), 8.77 (t, J = 5.7 Hz, 1H), 8.47 (d, J = 2.0 Hz, 1H), 8.35 (s, 1H), 8.31 (d, J = 0.9 Hz, 1H), 8.23 (s, 1H), 8.17 (d, J = 9.0 Hz, 1H), 8.06 (br. s, 1H), 7.97 (d, J = 8.4 Hz, 1H), 7.95 (q, J = 1.0 Hz, 1H), 7.32 (br. s, 1H), 7.23 (d, J = 7.4 Hz, 1H), 7.18 (td, J = 7.6, 1.2 Hz, 1H), 7.08 (td, J = 7.0, 1.0 Hz, 1H), 4.35 (d, J = 5.3 Hz, 2H), 2.16 (s, 3H). | 514.4 | B | B |
| I- 700 | 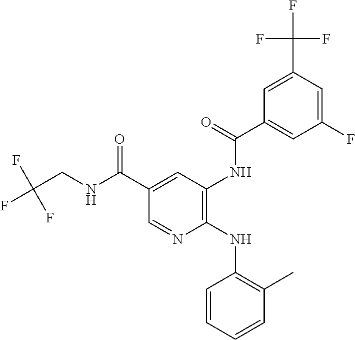 [SMILES: Cc1ccccc1Nc1ncc(C(=O)NCC(F)(F)F)cc1NC(=O)c1cc(F)cc(C(F)(F)F)c1] | (500 MHz, DMSO) 10.29 (s, 1H), 8.93 (t, J = 5.9 Hz, 1H), 8.54 (s, 1H), 8.49 (d, J = 2.2 Hz, 1H), 8.46 (s, 1H), 8.35 (s, 1H), 8.23 (s, 1H), 8.17 (d, J = 9.1 Hz, 1H), 8.05 (s, 1H), 7.98 (d, J = 8.4 Hz, 1H), 7.71 (d, J = 7.8 Hz, 1H), 7.35 (dd, J = 7.5, 4.8 Hz, 1H), 7.30 (d, J = 7.5 Hz, 1H), 7.23 (d, J = 7.4 Hz, 1H), 7.18 (td, J = 7.6, 1.2 Hz, 1H), 7.09 (td, J = 7.4, 1.1 Hz, 1H), 4.48 (d, J = 5.8 Hz, 2H), 2.16 (s, 3H). | 524.3 | B | D |
| I- 701 | 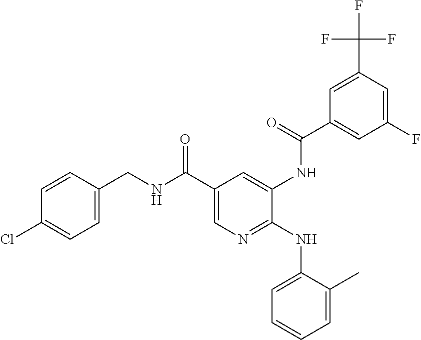 [SMILES: Cc1ccccc1Nc1ncc(C(=O)NCc2ccc(Cl)cc2)cc1NC(=O)c1cc(F)cc(C(F)(F)F)c1] | (500 MHz, DMSO) 10.30 (br. s, 1H), 8.90 (t, J = 6.3 Hz, 1H), 8.48 (d, J = 2.1 Hz, 1H), 8.45 (s, 1H), 8.24 (s, 1H), 8.17 (d, J = 9.0 Hz, 1H), 8.08 (s, 1H), 7.98 (d, J = 8.4 Hz, 1H), 7.30 (br. s, 1H), 7.24 (d, J = 7.4 Hz, 1H), 7.19 (td, J = 7.5, 1.2 Hz, 1H), 7.10 (td, J = 7.4, 1.0 Hz, 1H), 4.15-3.99 (qd, J = 10, 6.5 Hz, 2H), 2.16 (s, 3H). | 515.2 | B | B |
| I- 702 | 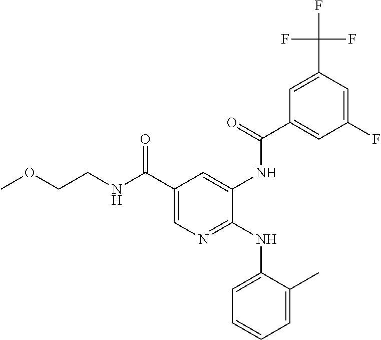 [SMILES: COCCNC(=O)c1cnc(Nc2ccccc2C)c(NC(=O)c2cc(F)cc(C(F)(F)F)c2)c1] | (400 MHz, dmso) 10.31 (s, 1H), 8.92 (t, J = 6.0 Hz, 1H), 8.51 (d, J = 2.2 Hz, 1H), 8.35 (s, 1H), 8.24 (s, 1H), 8.18 (d, J = 9.6 Hz, 1H), 8.07 (s, 1H), 8.00 (d, J = 8.7 Hz, 1H), 7.42-7.38 (m, 2H), 7.37- 7.30 (m, 3H), 7.25 (d, J = 7.1 Hz, 1H), 7.20 (td, J = 7.5, 1.4 Hz, 1H), 7.11 (td, J = 7.4, 1.3 Hz, 1H), 4.46 (d, J = 5.9 Hz, 2H), 2.18 (s, 3H) | 557.2 | C | |
| I- 703 | 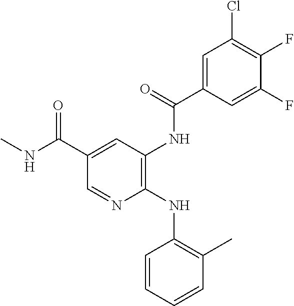 [SMILES: CNC(=O)c1cnc(Nc2ccccc2C)c(NC(=O)c2cc(F)c(F)c(Cl)c2)c1] | (400 MHz, dmso) 10.28 (s, 1H), 8.45 (d, J = 2.2 Hz, 1H), 8.37 (t, J = 5.3 Hz, 1H), 8.30 (s, 1H), 8.23 (s, 1H), 8.17 (d, J = 8.9 Hz, 1H), 8.03 (d, J = 2.2 Hz, 1H), 7.98 (d, J = 8.5 Hz, 1H), 7.31 (d, J = 7.5 Hz, 1H), 7.23 (d, J = 7.4 Hz, 1H), 7.18 (td, J = 7.4, 1.2 Hz, 1H), 7.09 (td, J = 7.4, 1.3 Hz, 1H), 3.47-3.37 (m, 4H), 3.26 (s, 3H), 2.15 (s, 3H). | 491.2 | B | A |
| I- 704 | 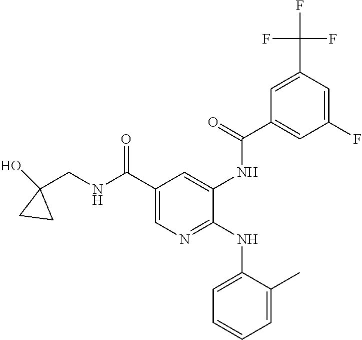 [SMILES: Cc1ccccc1Nc1ncc(C(=O)NCC2(O)CC2)cc1NC(=O)c1cc(F)cc(C(F)(F)F)c1] | (500 MHz, DMSO) 10.15 (s, 1H).8.40 (d, J = 2.2 Hz, 1H), 8.31-8.23 (m, 2H), 8.12 (m, J = 6.1 Hz, 1H), 8.08 (ddd, J = 10.6, 7.0, 2.0 Hz, 1H), 8.01 (d, J = 1.8 Hz, 1H), 7.34 (d, J = 7.6 Hz, 1H), 7.23 (d, J = 7.4 Hz, 1H), 7.18 (td, J = 7.5, 1.2 Hz, 1H), 7.08 (td, J = 7.4, 1.2 Hz, 1H), 3.32 (s, 1H), 2.76 (d, J = 4.5 Hz, 3H), 2.15 (s, 3H) | 431.2 | A | B |
| I- 705 | 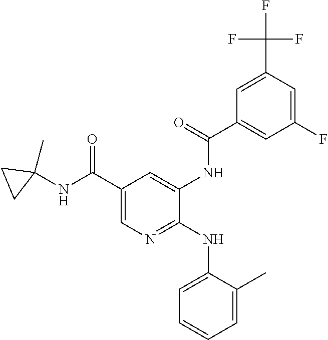 [SMILES: Cc1ccccc1Nc1ncc(C(=O)NC2(C)CC2)cc1NC(=O)c1cc(F)cc(C(F)(F)F)c1] | (500 MHz, DMSO) 8.47 (d, J = 2.2 Hz, 1H), 8.33 (s, 1H), 8.27 (t, J = 5.8 Hz, 1H), 8.24 (s, 1H), 8.17 (d, J = 9.2 Hz, 1H), 8.07 (d, J = 2.1 Hz, 1H), 7.97 (d, J = 8.5 Hz, 1H), 7.35 (d, J = 7.7 Hz, 1H), 7.23 (d, J = 7.4 Hz, 1H), 7.18 (td, J = 7.5, 1.2 Hz, 1H), 7.08 (td, J = 7.4, 1.2 Hz, 1H), 3.41 (d, J = 5.7 Hz, 2H), 3.33 (s, 2H), 2.16 (s, 3H), 0.54 (s, 4H). | 503.3 | A | C |
| I- 706 | 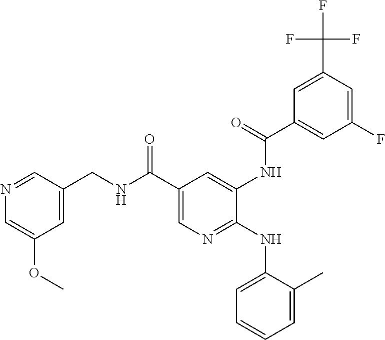 [SMILES: COc1cncc(CNC(=O)c2cnc(Nc3ccccc3C)c(NC(=O)c3cc(F)cc(C(F)(F)F)c3)c2)c1] | (500 MHz, DMSO) 10.26 (s, 1H), 8.47 (s, 1H), 8.39 (d, J = 2.0 Hz, 1H), 8.28 (s, 1H), 8.23 (s, 1H), 8.16 (d, J = 9.1 Hz, 1H), 8.04-7.92 (m, 2H), 7.32 (s, 1H), 7.22 (d, J = 7.0 Hz, 1H), 7.17 (td, J = 7.4, 1.2 Hz, 1H), 7.07 (td, J = 7.3, 0.8 Hz, 1H), 2.15 (s, 3H), 1.35 (s, 3H), 0.71 (dd, J = 4.9, 4.3 Hz, 2H), 0.59 (dd, J = 5.1, 4.5 Hz, 2H) | 487.2 | A | B |
| I- 707 | 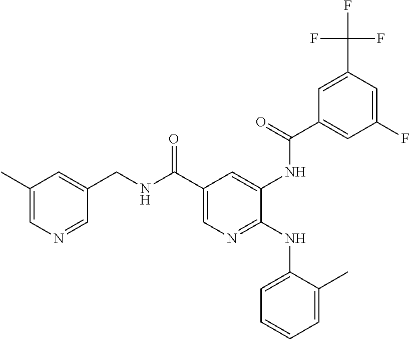 [SMILES: Cc1cncc(CNC(=O)c2cnc(Nc3ccccc3C)c(NC(=O)c3cc(F)cc(C(F)(F)F)c3)c2)c1] | (400 MHz, dmso) 10.25 (s, 1H), 8.86 (t, J = 5.8 Hz, 1H), 8.45 (d, J = 2.0 Hz, 1H), 8.30 (s, 1H), 8.19 (s, 1H), 8.13 (dd, J = 14.4, 2.3 Hz, 3H), 8.01 (s, 1H), 7.94 (d, J = 8.3 Hz, 1H), 7.27 (dd, J = 2.7, 1.8 Hz, 2H), 7.20 (d, J = 7.6 Hz, 1H), 7.14 (td, J = 7.5, 1.3 Hz, 1H), 7.05 (td, J = 7.1, 1.0 Hz, 1H), 4.44 (d, J = 5.8 Hz, 2H), 3.77 (s, 3H), 2.12 (s, 3H) | 554.3 | D | |
| I- 708 | 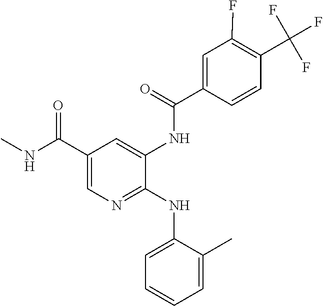 [SMILES: CNC(=O)c1cnc(Nc2ccccc2C)c(NC(=O)c2ccc(C(F)(F)F)c(F)c2)c1] | (500 MHz, DMSO) 10.31 (s, 1H), 8.91 (dd, J = 9.6, 3.6 Hz, 1H), 8.50 (d, J = 2.1 Hz, 1H), 8.36 (d, J = 1.7 Hz, 2H), 8.31 (d, J = 1.6 Hz, 1H), 8.24 (s, 1H), 8.18 (d, J = 9.1 Hz, 1H), 8.07 (s, 1H), 7.99 (d, J = 8.7 Hz, 1H), 7.54 (s, 1H), 7.32 (d, J = 7.0 Hz, 1H), 7.25 (d, J = 7.5 Hz, 1H), 7.20 (t, J = 7.6 Hz, 1H), 7.11 (t, J = 7.4 Hz, 1H), 4.47 (d, J = 5.8 Hz, 2H), 2.30 (s, 3H), 2.18 (s, 3H). | 538.4 | D | |
| I- 709 | 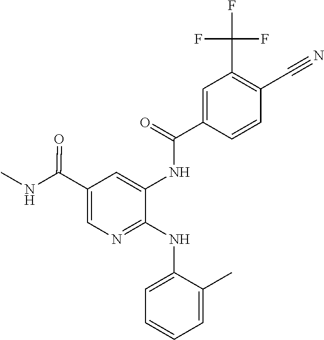 [SMILES: CNC(=O)c1cnc(Nc2ccccc2C)c(NC(=O)c2ccc(C#N)c(C(F)(F)F)c2)c1] | (500 MHz, DMSO-d6) 10.15 (s, 1H), 8.42 (d, J = 2.2 Hz, 1H), 8.29-8.26 (m, 2H), 8.13 (d, J = 11.8 Hz, 1H), 8.06- 7.97 (m, 3H), 7.34 (d, J = 7.7 Hz, 1H), 7.23 (d, J = 7.5 Hz, 1H), 7.18 (td, J = 7.5, 1.2 Hz, 1H), 7.08 (td, J = 7.4, 1.2 Hz, 1H), 2.76 (d, J = 4.5 Hz, 3H), 2.15 (s, 3H). | 447.4 | D | |
| I- 710 | 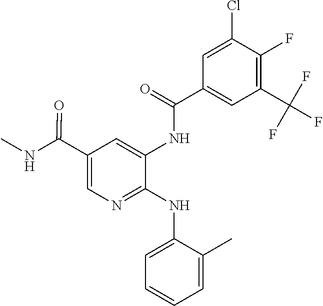 [SMILES: CNC(=O)c1cnc(Nc2ccccc2C)c(NC(=O)c2cc(Cl)c(F)c(C(F)(F)F)c2)c1] | (500 MHz, DMSO-d6) 10.40 (s, 1H), 8.56 (s, 1H), 8.47 (d, J = 8.0 Hz, 1H), 8.42 (d, J = 2.1 Hz, 1H), 8.38 (d, J = 8.0 Hz, 1H), 8.31 (s, 1H), 8.27 (q, J = 4.2 Hz, 1H), 8.02 (d, J = 1.5 Hz, 1H), 7.33 (d, J = 7.8 Hz, 1H), 7.23 (d, J = 7.5 Hz, 1H), 7.18 (td, J = 7.6, 1.2 Hz, 1H), 7.08 (td, J = 7.4, 1.0 Hz, 1H), 2.76 (d, J = 4.5 Hz, 3H), 2.16 (s, 3H). | 454.3 | C | |
| I- 711 | 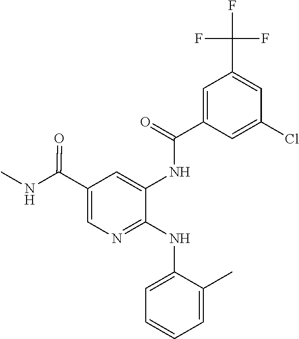 [SMILES: CNC(=O)c1cnc(Nc2ccccc2C)c(NC(=O)c2cc(Cl)cc(C(F)(F)F)c2)c1] | (500 MHz, DMSO-d6) 10.28 (s, 1H), 8.57 (dd, J = 6.5, 1.7 Hz, 1H), 8.41 (d, J = 2.2 Hz, 1H), 8.36 (dd, J = 6.0, 1.5 Hz, 1H), 8.29 (s, 1H), 8.27 (q, J = 4.4 Hz, 1H), 8.00 (d, J = 1.4 Hz, 1H), 7.32 (d, J = 7.4 Hz, 1H), 7.23 (d, J = 7.3 Hz, 1H), 7.18 (td, J = 7.6, 1.1 Hz, 1H), 7.08 (td, J = 7.5, 1.0 Hz, 1H), 2.76 (d, J = 4.5 Hz, 3H), 2.15 (s, 3H). | 481.2 | A | C |
| I- 712 | 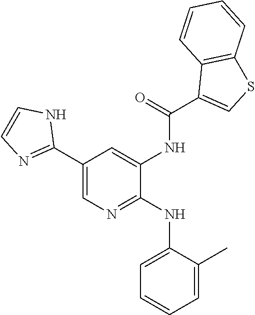 [SMILES: Cc1ccccc1Nc1ncc(-c2ncc[nH]2)cc1NC(=O)c1csc2ccccc12] | (500 MHz, DMSO-d6) 10.27 (s, 1H), 8.41 (d, J = 2.1 Hz, 1H), 8.38 (s, 1H), 8.30 (s, 2H), 8.26 (q, J = 4.1 Hz, 1H), 8.13 (s, 1H), 8.01 (s, 1H), 7.35-7.30 (m, 1H), 7.23 (d, J = 7.4 Hz, 1H), 7.18 (td, J = 7.6, 1.1 Hz, 1H), 7.08 (td, J = 7.5, 0.9 Hz, 1H), 2.76 (d, J = 4.5 Hz, 3H), 2.16 (s, 3H). | 463.2 | B | C |
| I- 713 | 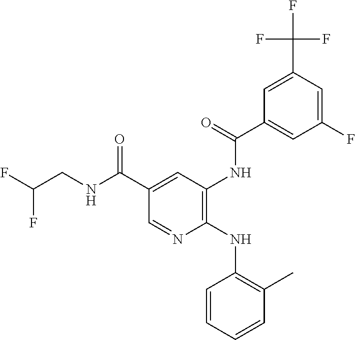 [SMILES: Cc1ccccc1Nc1ncc(C(=O)NCC(F)F)cc1NC(=O)c1cc(F)cc(C(F)(F)F)c1] | (400 MHz, DMSO) 12.32 (s, 1H), 10.16 (s, 1H), 8.69 (br s) and 8.53 (br s) and 8.37 (br s, 5H), 8.04 (d, J = 7.8 Hz, 1H), 7.57 (br s, 1H), 7.43 (dt, J = 14.7, 7.0 Hz, 2H), 7.16 (m, 3H), 7.00 (br s, 1H), 6.95-6.84 (m, 1H), 2.23 (s, 3H). | 426.3 | B | D |
| I- 714 | 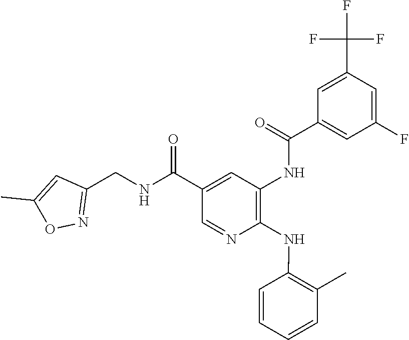 [SMILES: Cc1cc(CNC(=O)c2cnc(Nc3ccccc3C)c(NC(=O)c3cc(F)cc(C(F)(F)F)c3)c2)no1] | (500 MHz, DMSO) 10.29 (br. s, 1H), 8.69 (t, J = 5.9 Hz, 1H), 8.47 (d, J = 2.1 Hz, 1H), 8.40 (br. s, 1H), 8.23 (s, 1H), 8.17 (d, J = 9.0 Hz, 1H), 8.05 (br. s, 1H), 7.98 (d, J = 8.3 Hz, 1H), 7.30 (br. d, J = 5.7 Hz, 1H), 7.24 (d, J = 7.4 Hz, 1H), 7.19 (td, J = 7.5, 1.2 Hz, 1H), 7.10 (td, J = 7.4, 0.7 Hz, 1H), 6.10 (tt, J = 56.1, 4.0 Hz, 1H), 3.65 (tdd, J = 15.6, 5.6, 4.2 Hz, 2H), 2.15 (s, 3H). | 497.3 | B | B |
| I- 715 | 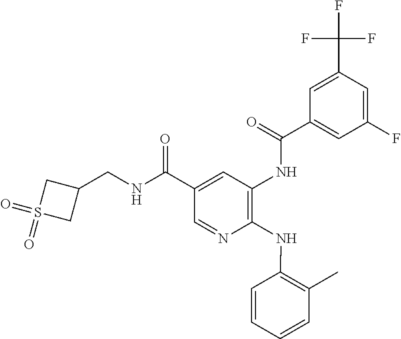 [SMILES: Cc1ccccc1Nc1ncc(C(=O)NCC2CS(=O)(=O)C2)cc1NC(=O)c1cc(F)cc(C(F)(F)F)c1] | (500 MHz, DMSO) 10.27 (br. s, 1H), 8.89 (t, J = 5.8 Hz, 1H), 8.45-8.36 (m, 2H), 8.24 (s, 1H), 8.17 (d, J = 9.1 Hz, 1H), 8.10 (br. s, 1H), 7.96 (d, J = 7.3 Hz, 1H), 7.37 (br. s, 1H), 7.23 (d, J = 7.4 Hz, 1H), 7.18 (td, J = 7.5, 1.1 Hz, 1H), 7.08 (t, J = 7.5 Hz, 1H), 6.15 (s, 1H), 4.43 (d, J = 5.9 Hz, 2H), 2.36 (d, J = 0.8 Hz, 3H), 2.17 (s, 3H). | 528.2 | D | |
| I- 716 | 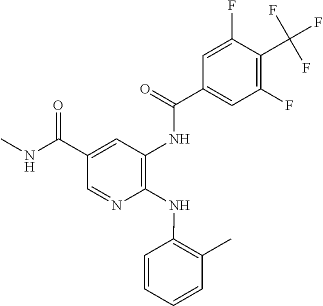 [SMILES: CNC(=O)c1cnc(Nc2ccccc2C)c(NC(=O)c2cc(F)c(C(F)(F)F)c(F)c2)c1] | (500 MHz, DMSO) 10.20 br. (s, 1H), 8.59 (t, J = 5.8 Hz, 1H), 8.43 (d, J = 2.2 Hz, 1H), 8.38 (s, 1H), 8.24 (s, 1H), 8.17 (d, J = 8.9 Hz, 1H), 8.03 (s, 1H), 7.97 (d, J = 8.5 Hz, 1H), 7.33 (d, J = 7.7 Hz, 1H), 7.23 (d, J = 7.4 Hz, 1H), 7.18 (td, J = 7.5, 1.2 Hz, 1H), 7.09 (td, J = 7.4, 1.2 Hz, 1H), 4.29-4.20 (m, 2H), 3.99-3.91 (m, 2H), 3.54-3.45 (m, 2H), 2.76 (mt, 1H), 2.16 (s, 3H). | 551.2 | D | |
| I- 717 | 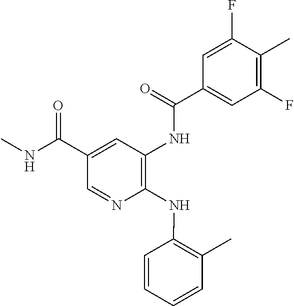 | (500 MHz, DMSO) 10.29 (br. s, 1H), 8.41 (d, J = 2.2 Hz, 1H), 8.30 (s, 1H), 8.27 (q, J = 4.5 Hz, 1H), 8.02 (d, J = 1.9 Hz, 1H), 8.00 (s, 1H), 7.98 (s, 1H), 7.32 (d, J = 7.7 Hz, 1H), 7.24 (d, J = 7.5 Hz, 1H), 7.19 (td, J = 7.6, 1.2 Hz, 1H), 7.09 (td, J = 7.4, 1.2 Hz, 1H), 2.76 (d, J = 4.5 Hz, 3H), 2.15 (s, 3H). | 465.3 | C | |
| I- 718 | 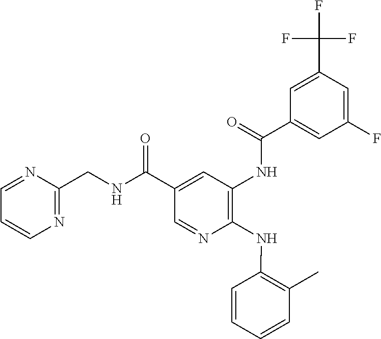 [SMILES: Cc1ccccc1Nc1ncc(C(=O)NCc2ncccn2)cc1NC(=O)c1cc(F)cc(C(F)(F)F)c1] | (500 MHz, DMSO) 10.06 (br. s, 1H), 8.42 (d, J = 2.2 Hz, 1H), 8.28 (q, J = 4.3 Hz, 1H), 8.21 (s, 1H), 8.01 (d, J = 2.2 Hz, 1H), 7.77-7.66 (m, 2H), 7.37 (d, J = 7.4 Hz, 1H), 7.22 (d, J = 7.4 Hz, 1H), 7.18 (td, J = 7.5, 1.2 Hz, 1H), 7.07 (td, J = 7.4, 1.2 Hz, 1H), 2.76 (d, J = 4.5 Hz, 3H), 2.24 (s, 3H), 2.15 (s, 3H). | 411.1 | B | |
| I- 719 | 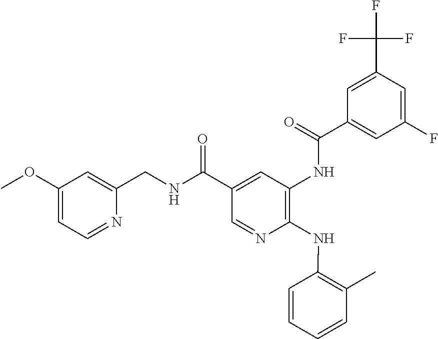 [SMILES: COc1ccnc(CNC(=O)c2cnc(Nc3ccccc3C)c(NC(=O)c3cc(F)cc(C(F)(F)F)c3)c2)c1] | (500 MHz, DMSO) 10.27 (br. s, 1H), 8.90 (t, J = 5.7 Hz, 1H), 8.76 (d, J = 4.9 Hz, 2H), 8.50 (d, J = 2.0 Hz, 1H), 8.38 (br. s, 1H), 8.24 (s, 1H), 8.17 (d, J = 9.3 Hz, 1H), 8.10 (br. s, 1H), 7.96 (d, J = 8.2 Hz, 1H), 7.39 (t, J = 4.5 Hz, 1H), 7.36 (br. s, 1H), 7.24 (d, J = 7.2 Hz, 1H), 7.19 (td, J = 7.5, 1.2 Hz, 1H), 7.09 (t, J = 7.5 Hz, 1H), 4.64 (d, J = 5.8 Hz, 2H), 2.17 (s, 3H). | 525.2 | D | |
| I- 720 | 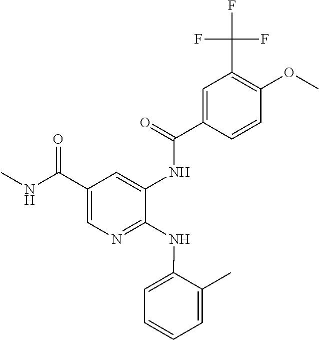 | (500 MHz, DMSO) 10.31 (br. s, 1H), 8.89 (t, J = 5.9 Hz, 1H), 8.51 (s, 1H), 8.37 (br. s, 1H), 8.32 (d, J = 6.1 Hz, 1H), 8.24 (s, 1H), 8.17 (d, J = 9.3 Hz, 1H), 8.10 (br. s, 1H), 7.97 (d, J = 7.6 Hz, 1H), 7.34 (br. s, 1H), 7.24 (d, J = 7.2 Hz, 1H), 7.18 (td, J = 7.7, 1.1 Hz, 1H), 7.09 (t, J = 7.5 Hz, 1H), 6.87-6.85 (m, 2H), 4.50 (d, J = 5.9 Hz, 2H), 3.80 (s, 3H), 2.17 (s, 3H). | 554.2 | D | |
| I- 721 | 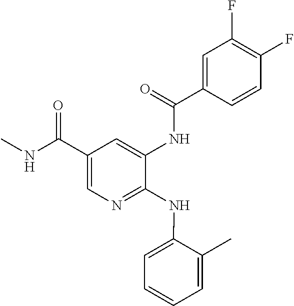 [SMILES: CNC(=O)c1cnc(Nc2ccccc2C)c(NC(=O)c2ccc(F)c(F)c2)c1] | (500 MHz, DMSO-d6) 10.11 (br. s, 1H), 8.43 (s, 1H), 8.35-8.26 (m, 3H), 8.16 (s, 1H), 7.99 (s, 1H), 7.43 (d, J = 9.2 Hz, 1H), 7.41 (d, J = 8.1 Hz, 1H), 7.22 (d, J = 7.5 Hz, 1H), 7.17 (t, J = 7.3 Hz, 1H), 7.05 (t, J = 6.7 Hz, 1H), 3.99 (s, 3H), 2.77 (d, J = 2.2 Hz, 3H), 2.15 (s, 3H). | 459.4 | D | |
| I- 722 | 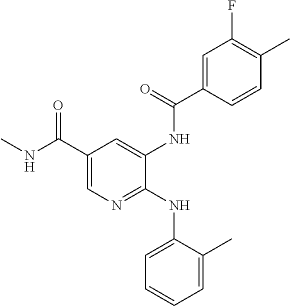 | (500 MHz, DMSO-d6) 10.06 (br. s, 1H), 8.42 (s, 1H), 8.28 (d, J = 3.2 Hz, 1H), 8.22 (s, 1H), 8.15-8.07 (m, 1H), 8.01 (s, 1H), 7.95-7.89 (m, 1H), 7.67- 7.60 (m, 1H), 7.36 (d, J = 7.4 Hz, 1H), 7.22 (d, J = 7.3 Hz, 1H), 7.18 (t, J = 7.3 Hz, 1H), 7.07 (t, J = 6.9 Hz, 1H), 2.76 (d, J = 3.3 Hz, 3H), 2.15 (s, 3H). | 397.2 | D | |
| I- 723 | 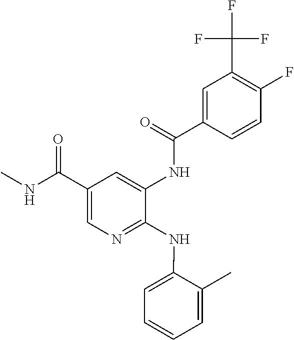 [SMILES: CNC(=O)c1cnc(Nc2ccccc2C)c(NC(=O)c2ccc(F)c(C(F)(F)F)c2)c1] | (500 MHz, DMSO-d6) 10.01 (s, 1H), 8.42 (s, 1H), 8.31-8.26 (m, 1H), 8.15 (s, 1H), 8.02 (s, 1H), 7.83-7.78 (m, 2H), 7.46 (t, J = 7.5 Hz, 1H), 7.41 (d, J = 7.6 Hz, 1H), 7.22 (d, J = 7.1 Hz, 1H), 7.17 (t, J = 7.3 Hz, 1H), 7.06 (t, J = 7.1 Hz, 1H), 2.76 (d, J = 3.2 Hz, 3H), 2.32 (s, 3H), 2.15 (s, 3H). | 393.2 | D | |
| I- 724 | 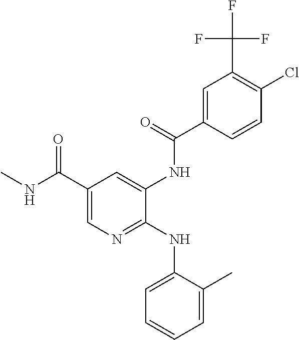 [SMILES: CNC(=O)c1cnc(Nc2ccccc2C)c(NC(=O)c2ccc(Cl)c(C(F)(F)F)c2)c1] | (500 MHz, DMSO-d6) 10.29 (s, 1H), 8.47 (s, 1H), 8.42 (s, 1H), 8.31-8.27 (m, 2H), 8.24 (s, 1H), 7.99 (s, 1H), 7.94 (d, J = 8.1 Hz, 1H), 7.33 (d, J = 7.2 Hz, 1H), 7.22 (d, J = 7.0 Hz, 1H), 7.17 (t, J = 7.3 Hz, 1H), 7.07 (t, J = 7.0 Hz, 1H), 2.76 (d, J = 3.2 Hz, 3H), 2.15 (s, 3H). | 447.1 | D | |
| I- 725 | 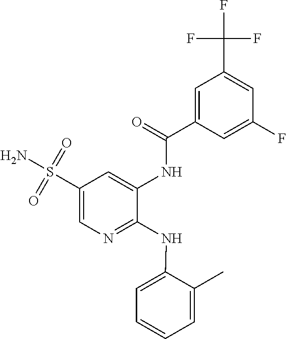 [SMILES: Cc1ccccc1Nc1ncc(S(N)(=O)=O)cc1NC(=O)c1cc(F)cc(C(F)(F)F)c1] | (500 MHz, DMSO-d6) 10.29 (s, 1H), 8.47 (s, 1H), 8.42 (s, 1H), 8.31-8.27 (m, 2H), 8.24 (s, 1H), 7.99 (s, 1H), 7.94 (d, J = 8.1 Hz, 1H), 7.33 (d, J = 7.2 Hz, 1H), 7.22 (d, J = 7.0 Hz, 1H), 7.17 (t, J = 7.3 Hz, 1H), 7.07 (t, J = 7.0 Hz, 1H), 2.76 (d, J = 3.2 Hz, 3H), 2.15 (s, 3H). | 463.1 | D | |
| I- 726 | 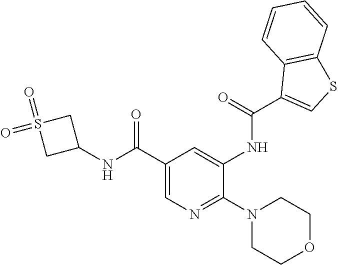 [SMILES: O=C(NC1CS(=O)(=O)C1)c1cnc(N2CCOCC2)c(NC(=O)c2csc3ccccc23)c1] | (400 MHz, DMSO) 8.65 (s, 1H), 8.25 (s, 2H), 8.17 (d, J = 8.9 Hz, 1H), 8.09- 7.79 (br m, 2H), 7.71-7.40 (br m, 1H), 7.28 (s, 1H), 7.25 (d, J = 7.3 Hz, 1H), 7.20 (t, J = 8.2 Hz, 1H), 7.10 (t, J = 7.4 Hz, 1H), 2.18 (s, 3H). | 469.2 | D | |
| I- 727 | 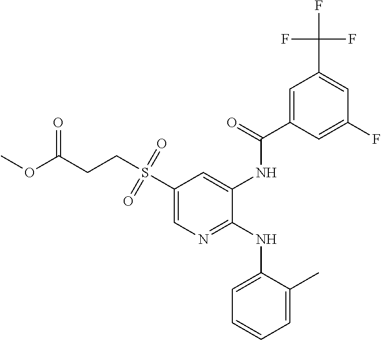 [SMILES: COC(=O)CCS(=O)(=O)c1cnc(Nc2ccccc2C)c(NC(=O)c2cc(F)cc(C(F)(F)F)c2)c1] | (400 MHz, dmso) 9.99 (s, 1H), 9.08 (d, J = 3.8 Hz, 1H), 8.64 (s, 1H), 8.63 (d, J = 2.2 Hz, 1H), 8.53-8.48 (m, 1H), 8.25 (d, J = 2.2 Hz, 1H), 8.12-8.07 (m, 1H), 7.52-7.42 (m, 2H), 4.62-4.53 (m, 3H), 4.34-4.24 (m, 2H), 3.72-3.65 (m, 4H), 3.44-3.36 (m, 4H). | 487.2 | D | |
| I- 728 | 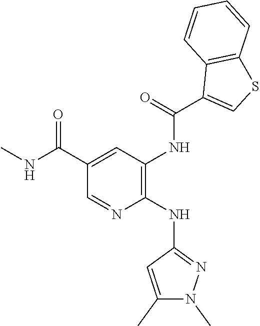 [SMILES: CNC(=O)c1cnc(Nc2cc(C)n(C)n2)c(NC(=O)c2csc3ccccc23)c1] | (400 MHz, DMSO) 10.37 (s, 1H), 8.88 (s, 1H), 8.28 (s, 1H), 8.24 (s, 1H), 8.18 (d, J = 9.1 Hz, 1H), 7.94 (dd, J = 23.3, 8.0 Hz, 2H), 7.27 (d, J = 7.5 Hz, 2H), 7.21 (t, J = 6.8 Hz, 1H), 7.15 (t, J = 7.6 Hz, 1H), 3.56 (s, 3H), 3.56-3.50 (m, 2H), 2.67 (t, J = 7.2 Hz, 2H), 2.18 (s, 3H). | 540.3 | D | |
| I- 729 | 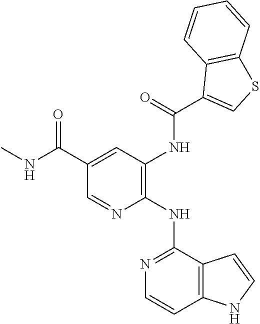 [SMILES: CNC(=O)c1cnc(Nc2nccc3[nH]ccc23)c(NC(=O)c2csc3ccccc23)c1] | | | D | |
| I- 730 | 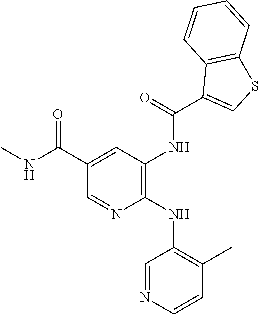 [SMILES: CNC(=O)c1cnc(Nc2cnccc2C)c(NC(=O)c2csc3ccccc23)c1] | | | D | |
| I- 731 | 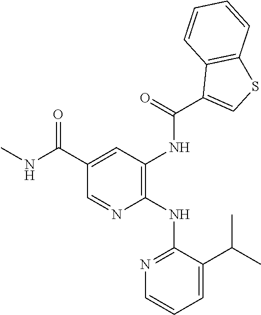 [SMILES: CNC(=O)c1cnc(Nc2ncccc2C(C)C)c(NC(=O)c2csc3ccccc23)c1] | | | D | |
| I- 732 | 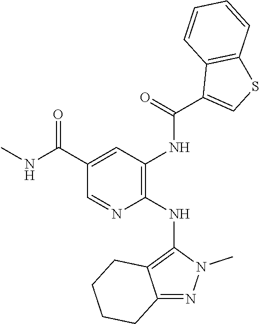 [SMILES: CNC(=O)c1cnc(Nc2c3c(nn2C)CCCC3)c(NC(=O)c2csc3ccccc23)c1] | | | D | |
| I- 733 | 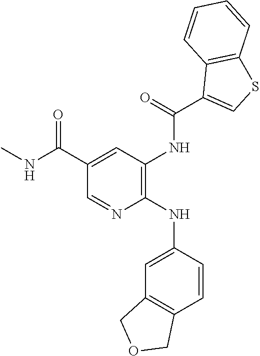 [SMILES: CNC(=O)c1cnc(Nc2ccc3c(c2)COC3)c(NC(=O)c2csc3ccccc23)c1] | | | D | |
| I- 734 | 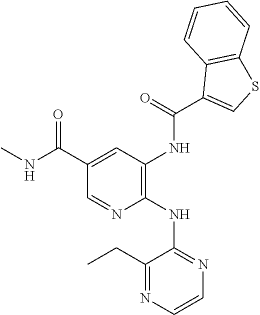 [SMILES: CCc1nccnc1Nc1ncc(C(=O)NC)cc1NC(=O)c1csc2ccccc12] | | | D | |
| I- 735 | 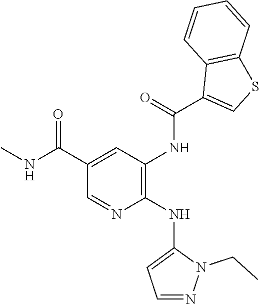 [SMILES: CCn1nccc1Nc1ncc(C(=O)NC)cc1NC(=O)c1csc2ccccc12] | | | D | |
| I- 736 | 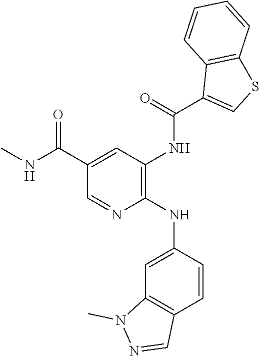 [SMILES: CNC(=O)c1cnc(Nc2ccc3cnn(C)c3c2)c(NC(=O)c2csc3ccccc23)c1] | | | D | |
| I- 737 | 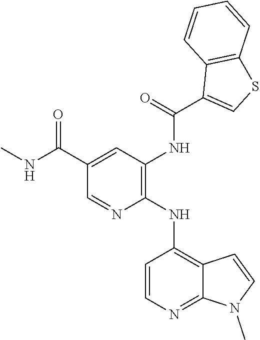 [SMILES: CNC(=O)c1cnc(Nc2ccnc3c2ccn3C)c(NC(=O)c2csc3ccccc23)c1] | | | D | |
| I- 738 | 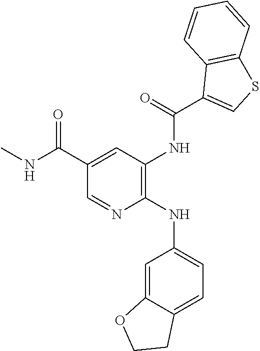 [SMILES: CNC(=O)c1cnc(Nc2ccc3c(c2)OCC3)c(NC(=O)c2csc3ccccc23)c1] | | | D | |
| I- 739 | 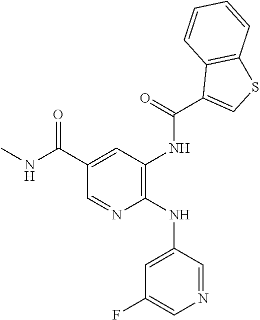 [SMILES: CNC(=O)c1cnc(Nc2cncc(F)c2)c(NC(=O)c2csc3ccccc23)c1] | | | D | |
| I- 740 | 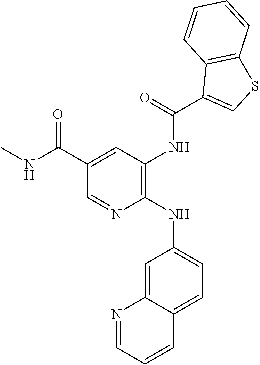 [SMILES: CNC(=O)c1cnc(Nc2ccc3cccnc3c2)c(NC(=O)c2csc3ccccc23)c1] | | | D | |
| I- 741 | 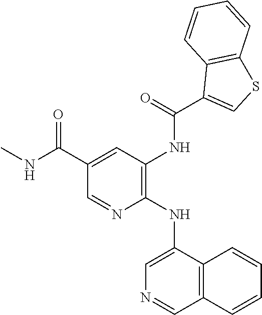 [SMILES: CNC(=O)c1cnc(Nc2cncc3ccccc23)c(NC(=O)c2csc3ccccc23)c1] | | | D | |
| I- 742 | 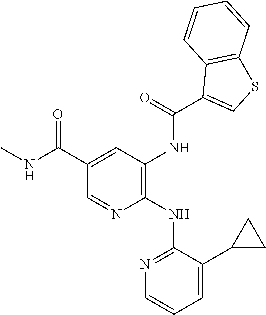 [SMILES: CNC(=O)c1cnc(Nc2ncccc2C2CC2)c(NC(=O)c2csc3ccccc23)c1] | | | D | |
| I- 743 | 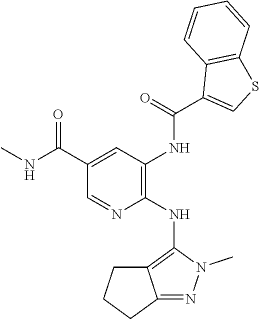 [SMILES: CNC(=O)c1cnc(Nc2c3c(nn2C)CCC3)c(NC(=O)c2csc3ccccc23)c1] | | | D | |
| I- 744 | 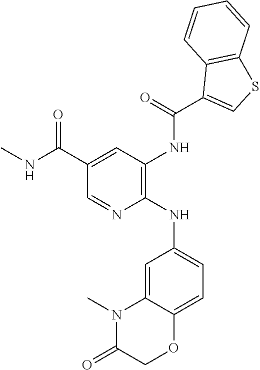 [SMILES: CNC(=O)c1cnc(Nc2ccc3c(c2)N(C)C(=O)CO3)c(NC(=O)c2csc3ccccc23)c1] | | | D | |
| I- 745 | 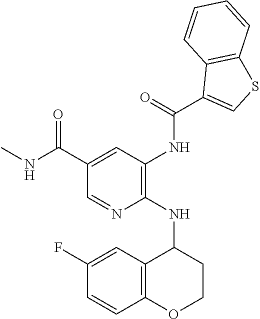 [SMILES: CNC(=O)c1cnc(NC2CCOc3ccc(F)cc32)c(NC(=O)c2csc3ccccc23)c1] | | | B | C |
| I- 746 | 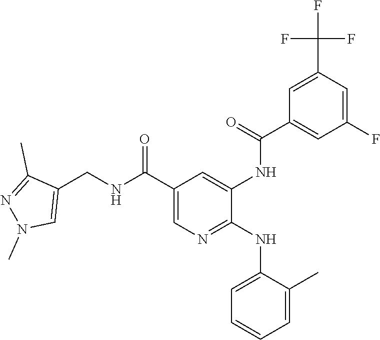 [SMILES: Cc1ccccc1Nc1ncc(C(=O)NCc2cn(C)nc2C)cc1NC(=O)c1cc(F)cc(C(F)(F)F)c1] | | | D | |
| I- 747 | 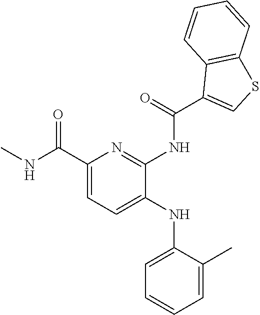 [SMILES: CNC(=O)c1ccc(Nc2ccccc2C)c(NC(=O)c2csc3ccccc23)n1] | (400 MHz, dmso) 8.53 (t, J = 5.4 Hz, 1H), 8.44 (d, J = 2.2 Hz, 1H), 8.31 (s, 1H), 8.23 (s, 1H), 8.16 (d, J = 9.1 Hz, 1H), 8.04 (d, J = 1.8 Hz, 1H), 7.98 (d, J = 8.3 Hz, 1H), 7.47 (s, 1H), 7.31 (d, J = 7.5 Hz, 1H), 7.23 (d, J = 7.7 Hz, 1H), 7.17 (td, J = 7.5, 1.4 Hz, 1H), 7.08 (td, J = 7.4, 1.2 Hz, 1H), 4.21 (d, J = 5.5 Hz, 2H), 3.69 (s, 3H), 2.15 (s, 3H), 2.11 (s, 3H). | 541.3 | D | |
| I- 748 | 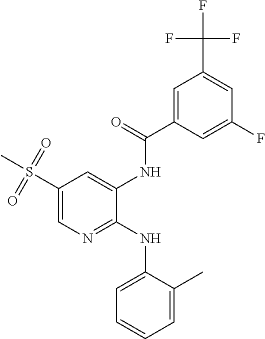 [SMILES: Cc1ccccc1Nc1ncc(S(C)(=O)=O)cc1NC(=O)c1cc(F)cc(C(F)(F)F)c1] | (400 MHz, DMSO) 10.60 (s, 1H), 8.73 (s, 1H), 8.53 (d, J = 7.4 Hz, 1H), 8.19 (q, J = 4.6 Hz, 1H), 8.09 (dd, J = 7.1, 1.2 Hz, 1H), 7.80 (d, J = 8.4 Hz, 1H), 7.61 (s, 1H), 7.54-7.40 (m, 2H), 7.27 (d, J = 7.4 Hz, 1H), 7.23-7.18 (m, 1H), 7.14 (dd, J = 8.1, 2.3 Hz, 2H), 7.07 (td, J = 7.4, 1.2 Hz, 1H), 2.80 (d, J = 4.9 Hz, 3H), 2.19 (s, 3H). | 417.3 | B | B |
| I- 749 | 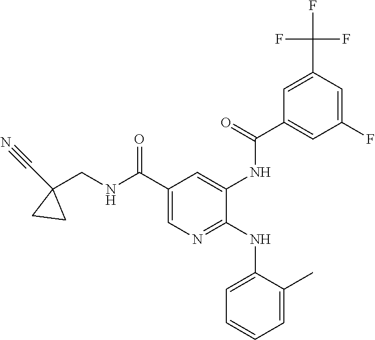 [SMILES: Cc1ccccc1Nc1ncc(C(=O)NCC2(C#N)CC2)cc1NC(=O)c1cc(F)cc(C(F)(F)F)c1] | (400 MHz, DMSO) 10.38 (s, 1H), 8.79 (s, 1H), 8.35 (d, J = 2.3 Hz, 1H), 8.23 (s, 1H), 8.18 (d, J = 9.0 Hz, 1H), 8.02 (s, 1H), 7.99 (d, J = 8.6 Hz, 1H), 7.27- 7.23 (m, 2H), 7.21 (td, J = 7.5, 1.4 Hz, 1H), 7.18-7.13 (m, 1H), 3.21 (s, 3H), 2.17 (s, 3H). | 468.2 | C | |
| I- 750 | 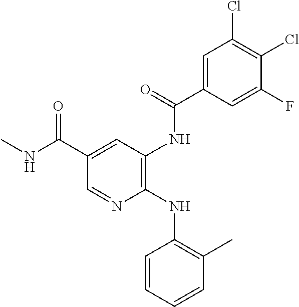 [SMILES: CNC(=O)c1cnc(Nc2ccccc2C)c(NC(=O)c2cc(F)c(Cl)c(Cl)c2)c1] | (400 MHz, dmso) 8.71 (t, J = 5.9 Hz, 1H), 8.46 (d, J = 2.1 Hz, 2H), 8.25 (s, 1H), 8.18 (d, J = 8.9 Hz, 1H), 8.12 (br s, 1H), 7.97 (d, J = 8.2 Hz, 1H), 7.39 (br s, 1H), 7.24 (d, J = 7.4 Hz, 1H), 7.18 (td, J = 7.6, 1.4 Hz, 1H), 7.08 (td, J = 7.4, 1.2 Hz, 1H), 3.41 (d, J = 6.0 Hz, 2H), 2.54 (s, 1H), 2.17 (s, 3H), 1.21 (dd, J = 7.2, 4.6 Hz, 2H), 1.11 (dd, J = 7.4, 5.0 Hz, 2H). | 512.3 | B | C |
| I- 751 | 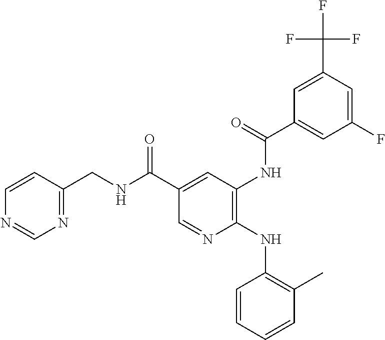 [SMILES: Cc1ccccc1Nc1ncc(C(=O)NCc2ccncn2)cc1NC(=O)c1cc(F)cc(C(F)(F)F)c1] | (400 MHz, dmso) 8.40 (dd, J = 5.5, 2.2 Hz, 3H), 8.29 (q, J = 4.2 Hz, 1H), 8.19 (t, J = 1.6 Hz, 1H), 8.09-8.03 (m, 2H), 7.37 (d, J = 6.9 Hz, 1H), 7.22 (d, J = 7.4 Hz, 1H), 7.17 (td, J = 7.5, 1.4 Hz, 1H), 7.07 (td, J = 7.4, 1.3 Hz, 1H), 2.76 (d, J = 4.5 Hz, 3H), 2.15 (s, 3H). | 447.2 | B | C |
| I- 752 | 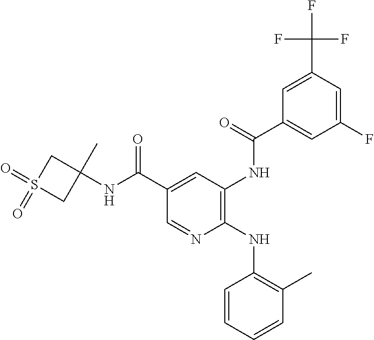 [SMILES: Cc1ccccc1Nc1ncc(C(=O)NC2(C)CS(=O)(=O)C2)cc1NC(=O)c1cc(F)cc(C(F)(F)F)c1] | (400 MHz, dmso) 9.11 (d, J = 1.3 Hz, 1H), 9.04 (br. t, J = 5.8 Hz, 1H), 8.73 (d, J = 5.3 Hz, 1H), 8.51 (d, J = 2.2 Hz, 1H), 8.48 (br. s, 1H), 8.35 (s, 1H), 8.25 (s, 1H), 8.18 (d, J = 9.3 Hz, 1H), 8.12 (d, J = 1.9 Hz, 1H), 7.97 (d, J = 8.4 Hz, 1H), 7.43 (dd, J = 5.2, 1.4 Hz, 1H), 7.37 (br. d, J = 8.1 Hz, 1H), 7.24 (d, J = 7.1 Hz, 1H), 7.19 (td, J = 7.4, 1.2 Hz, 1H), 7.09 (td, J = 7.4, 1.3 Hz, 1H), 4.53 (d, J = 5.8 Hz, 2H), 2.17 (s, 3H). | 525.2 | D | |
| I- 753 | 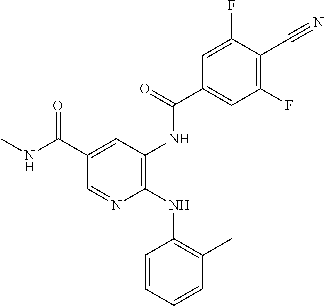 [SMILES: CNC(=O)c1cnc(Nc2ccccc2C)c(NC(=O)c2cc(F)c(C#N)c(F)c2)c1] | (400 MHz, dmso) 10.32 (br. s, 1H), 8.78 (s, 1H), 8.49 (br. s, 1H), 8.43 (d, J = 2.1 Hz, 1H), 8.25 (s, 1H), 8.17 (d, J = 9.0 Hz, 1H), 8.07 (br. s, 1H), 7.97 (d, J = 8.7 Hz, 1H), 7.35 (br. s, 1H), 7.24 (d, J = 7.4 Hz, 1H), 7.18 (td, J = 7.6, 1.3 Hz, 1H), 7.08 (td, J = 7.5, 0.9 Hz, 1H), 4.52 (mt, 2H), 4.27 (mt, 2H), 2.16 (s, 3H), 1.70 (s, 3H). | 551.2 | D | |
| I- 754 | 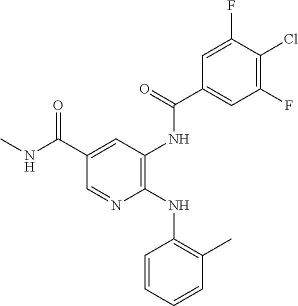 [SMILES: CNC(=O)c1cnc(Nc2ccccc2C)c(NC(=O)c2cc(F)c(Cl)c(F)c2)c1] | (400 MHz, dmso) 10.11 (br. s, 1H), 8.40 (d, J = 2.2 Hz, 1H), 8.36 (s, 1H), 8.27 (q, J = 4.2 Hz, 1H), 8.04 (d, J = 1.8 Hz, 1H), 8.01 (mt, 2H), 7.34 (d, J = 7.7 Hz, 1H), 7.24 (d, J = 7.5 Hz, 1H), 7.19 (td, J = 7.7, 1.4 Hz, 1H), 7.09 (td, J = 7.4, 1.2 Hz, 1H), 2.76 (d, J = 4.5 Hz, 3H), 2.15 (s, 3H). | 422.3 | B | B |
| I- 755 | 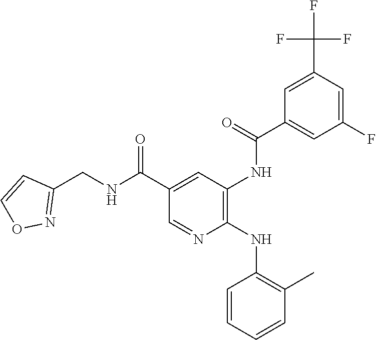 [SMILES: Cc1ccccc1Nc1ncc(C(=O)NCc2ccon2)cc1NC(=O)c1cc(F)cc(C(F)(F)F)c1] | (400 MHz, dmso) 10.01 (br. s, 1H), 8.41 (d, J = 2.2 Hz, 1H), 8.32-8.25 (m, 2H), 8.01 (d, J = 2.2 Hz, 1H), 7.97 (mt, 2H), 7.32 (d, J = 7.5 Hz, 1H), 7.23 (d, J = 7.5 Hz, 1H), 7.18 (td, J = 7.6, 1.5 Hz, 1H), 7.09 (td, J = 7.4, 1.3 Hz, 1H), 2.76 (d, J = 4.5 Hz, 3H), 2.14 (s, 3H). | 431.2 | D | |
| I- 756 | 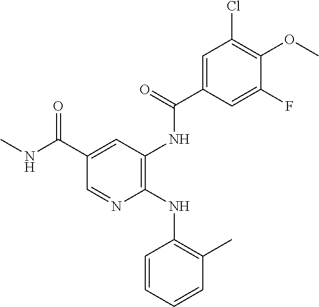 | (400 MHz, dmso) 8.92 (t, J = 6.0 Hz, 1H), 8.83 (d, J = 1.7 Hz, 1H), 8.50 (br. s, 1H), 8.44 (d, J = 1.6 Hz, 1H), 8.25 (s, 1H), 8.17 (d, J = 9.5 Hz, 1H), 8.14 (br. s, 1H), 7.94 (d, J = 8.2 Hz, 1H), 7.43 (br. s, 1H), 7.23 (d, J = 7.4 Hz, 1H), 7.18 (td, J = 7.5, 1.2 Hz, 1H), 7.07 (t, J = 7.3 Hz, 1H), 6.50 (d, J = 1.7 Hz, 1H), 4.53 (d, J = 5.8 Hz, 2H), 2.18 (s, 3H). | 512.3 | D | |
| I- 757 | 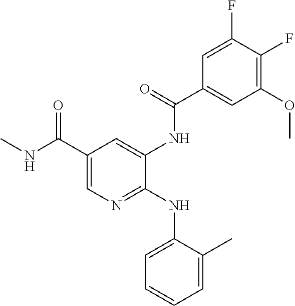 | (400 MHz, dmso) 10.07 (s, 1H), 8.41 (d, J = 2.2 Hz, 1H), 8.28 (q, J = 4.5 Hz, 1H), 8.23 (s, 1H), 8.04-8.01 (m, 1H), 7.98 (d, J = 2.2 Hz, 1H), 7.93 (dd, J = 12.0, 2.1 Hz, 1H), 7.33 (d, J = 7.8 Hz, 1H), 7.23 (d, J = 7.4 Hz, 1H), 7.18 (td, J = 7.6, 1.4 Hz, 1H), 7.08 (td, J = 7.4, 1.3 Hz, 1H), 4.01 (d, J = 2.0 Hz, 3H), 2.76 (d, J = 4.5 Hz, 3H), 2.14 (s, 3H). | 442.9 | A | B |
| I- 758 | 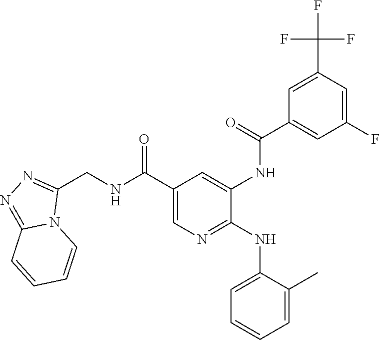 [SMILES: Cc1ccccc1Nc1ncc(C(=O)NCc2nnc3ccccn23)cc1NC(=O)c1cc(F)cc(C(F)(F)F)c1] | (400 MHz, DMSO-d6) 10.09 (s, 1H), 8.43 (d, J = 2.2 Hz, 1H), 8.29 (q, J = 4.3 Hz, 1H), 8.19 (s, 1H), 8.00 (d, J = 2.2 Hz, 1H), 7.71 (ddd, J = 10.8, 6.7, 1.9 Hz, 1H), 7.67 (dt, J = 7.2, 1.6 Hz, 1H), 7.38 (dd, J = 7.9, 0.9 Hz, 1H), 7.24-7.21 (m, 1H), 7.20-7.15 (m, 1H), 7.07 (td, J = 7.4, 1.3 Hz, 1H), 3.98 (s, 3H), 2.76 (d, J = 4.5 Hz, 3H), 2.16 (s, 3H). | 427.3 | D | |
| I- 759 | 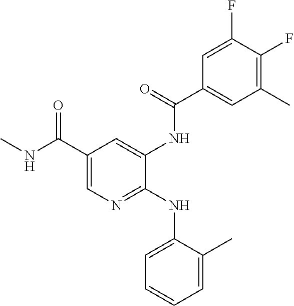 [SMILES: CNC(=O)c1cnc(Nc2ccccc2C)c(NC(=O)c2cc(C)c(F)c(F)c2)c1] | (400 MHz, dmso) 9.09 (t, J = 5.7 Hz, 1H), 8.55 (dt, J = 7.0, 1.1 Hz, 1H), 8.46 (d, J = 2.2 Hz, 1H), 8.42 (s, 1H), 8.22 (s, 1H), 8.16 (d, J = 9.2 Hz, 1H), 8.07 (s, 1H), 7.97 (d, J = 8.6 Hz, 1H), 7.76 (dt, J = 9.3, 1.1 Hz, 1H), 7.38 (ddd, J = 9.3, 6.6, 1.1 Hz, 1H), 7.29 (br s, 1H), 7.23 (d, J = 7.3 Hz, 1H), 7.17 (t, J = 6.8 Hz, 1H), 7.09 (t, J = 7.3 Hz, 1H), 7.02 (td, J = 6.8, 1.0 Hz, 1H), 5.01 (d, J = 5.6 Hz, 2H), 2.14 (s, 3H) + approx. 7% impurity | 564.3 | D | |
| I- 760 | 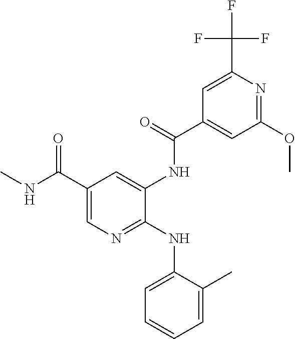 | 1H-NMR (400 MHz, DMSO-d6) 10.04 (br. s, 1H), 8.41 (d, J = 2.0 Hz, 1H), 8.29 (q, J = 3.6 Hz, 1H), 8.20 (s, 1H), 8.00 (d, J = 1.9 Hz, 1H), 7.96-7.89 (m, 1H), 7.82 (d, J = 5.8 Hz, 1H), 7.37 (d, J = 7.6 Hz, 1H), 7.22 (d, J = 7.5 Hz, 1H), 7.20-7.15 (m, 1H), 7.07 (td, J = 7.4, 0.9 Hz, 1H), 2.76 (d, J = 4.3 Hz, 3H), 2.37 (d, J = 1.3 Hz, 3H), 2.15 (s, 3H). | 411.2 | D | |
| I- 761 | 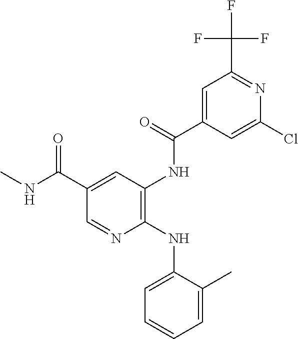 [SMILES: CNC(=O)c1cnc(Nc2ccccc2C)c(NC(=O)c2cc(Cl)nc(C(F)(F)F)c2)c1] | 1H-NMR (400 MHz, DMSO-d6) 10.18 (br. s, 1H), 8.41 (d, J = 2.2 Hz, 1H), 8.33 (s, 1H), 8.28 (q, J = 4.3 Hz, 1H), 8.02 (d, J = 2.1 Hz, 1H), 7.98 (d, J = 0.8 Hz, 1H), 7.71 (s, 1H), 7.33 (d, J = 7.7 Hz, 1H), 7.25-7.21 (m, 1H), 7.18 (td, J = 7.6, 1.2 Hz, 1H), 7.08 (td, J = 7.4, 1.3 Hz, 1H), 3.98 (s, 3H), 2.76 (d, J = 4.5 Hz, 3H), 2.15 (s, 3H). | 460.2 | C | |
| I- 762 | 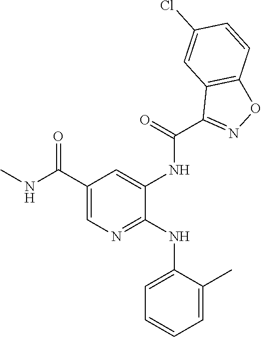 [SMILES: CNC(=O)c1cnc(Nc2ccccc2C)c(NC(=O)c2noc3ccc(Cl)cc23)c1] | 1H-NMR (400 MHz, DMSO-d6) 10.45 (br. s, 1H), 8.42-8.40 (m, 2H), 8.39- 8.37 (m, 1H), 8.36 (s, 1H), 8.28 (q, J = 4.3 Hz, 1H), 8.04 (s, 1H), 7.34-7.28 (m, 1H), 7.26-7.22 (m, 1H), 7.19 (td, J = 7.5, 1.3 Hz, 1H), 7.10 (td, J = 7.4, 1.3 Hz, 1H), 2.76 (d, J = 4.5 Hz, 3H), 2.16 (s, 3H). | 464.2 | C | |
| I- 763 | 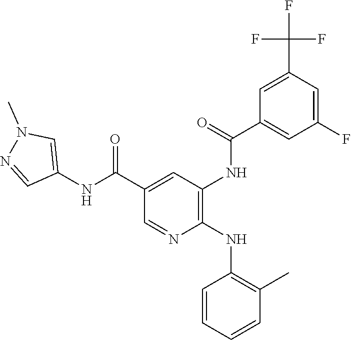 [SMILES: Cc1ccccc1Nc1ncc(C(=O)Nc2cnn(C)c2)cc1NC(=O)c1cc(F)cc(C(F)(F)F)c1] | (400 MHz, DMSO-d6) 10.50 (br. s, 1H), 8.42 (d, J = 2.2 Hz, 1H), 8.35 (br. s, 1H), 8.30 (q, J = 4.1 Hz, 1H), 8.14 (d, J = 2.1 Hz, 1H), 8.06 (d, J = 1.7 Hz, 1H), 7.99 (d, J = 9.0 Hz, 1H), 7.82 (dd, J = 9.0, 2.2 Hz, 1H), 7.37 (br. d, J = 7.3 Hz, 1H), 7.22 (d, J = 7.3 Hz, 1H), 7.17 (td, J = 7.5, 1.1 Hz, 1H), 7.07 (td, J = 7.4, 1.1 Hz, 1H), 2.76 (d, J = 4.5 Hz, 3H), 2.18 (s, 3H). | 436.3 | B | B |
| I- 764 | 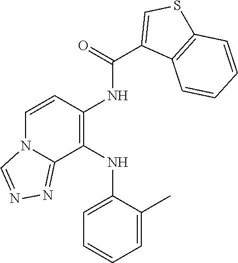 [SMILES: Cc1ccccc1Nc1c(NC(=O)c2csc3ccccc23)ccn2cnnc12] | (400 MHz, dmso) 10.21 (s, 1H), 8.49 (d, J = 2.2 Hz, 1H), 8.45 (br. s, 1H), 8.23 (s, 1H), 8.17-8.12 (m, 2H), 7.96- 7.93 (m, 2H), 7.50 (s, 1H), 7.35 (br. s, 1H), 7.21 (d, J = 7.5 Hz, 1H), 7.16 (td, J = 7.6, 1.2 Hz, 1H), 7.05 (td, J = 7.5, 1.1 Hz, 1H), 3.78 (s, 3H), 2.15 (s, 3H). | 513.3 | D | |
| I- 765 | 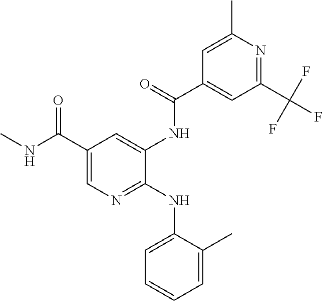 [SMILES: CNC(=O)c1cnc(Nc2ccccc2C)c(NC(=O)c2cc(C)nc(C(F)(F)F)c2)c1] | 1H-NMR (400 MHz, DMSO-d6) 10.24 (br. s, 1H), 8.41 (d, J = 2.2 Hz, 1H), 8.33 (br. s, 1H), 8.28 (q, J = 4.3 Hz, 1H), 8.23 (s, 1H), 8.11 (s, 1H), 8.02 (d, J = 1.5 Hz, 1H), 7.33 (br. d, J = 7.5 Hz, 1H), 7.23 (d, J = 7.4 Hz, 1H), 7.18 (td, J = 7.7, 1.4 Hz, 1H), 7.08 (td, J = 7.4, 1.1 Hz, 1H), 2.76 (d, J = 4.5 Hz, 3H), 2.67 (s, 3H), 2.16 (s, 3H). | 444.3 | B | B |
| I- 766 | 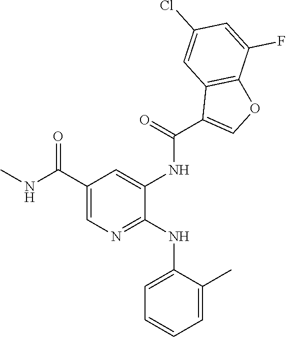 [SMILES: CNC(=O)c1cnc(Nc2ccccc2C)c(NC(=O)c2coc3c(F)cc(Cl)cc23)c1] | (400 MHz, dmso) 10.06 (s, 1H), 8.93 (s, 1H), 8.43 (d, J = 2.2 Hz, 1H), 8.31 (q, J = 4.4 Hz, 1H), 8.24 (s, 1H), 8.05 (d, J = 2.2 Hz, 1H), 7.94 (d, J = 2.0 Hz, 1H), 7.61 (dd, J = 10.7, 1.9 Hz, 1H), 7.38 (d, J = 6.9 Hz, 1H), 7.23 (d, J = 7.4 Hz, 1H), 7.18 (td, J = 7.8, 1.6 Hz, 1H), 7.08 (td, J = 7.4, 1.3 Hz, 1H), 2.77 (d, J = 4.5 Hz, 3H), 2.16 (s, 3H). | 453.2 | C | |
| I- 767 | 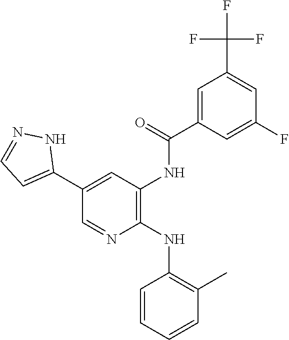 [SMILES: Cc1ccccc1Nc1ncc(-c2ccn[nH]2)cc1NC(=O)c1cc(F)cc(C(F)(F)F)c1] | (400 MHz, dmso) 10.42 (s, 1H), 8.42 (d, J = 2.0 Hz, 1H), 8.28 (s, 1H), 8.23 (s, 1H), 8.17 (br. d, J = 9.0 Hz, 1H), 8.02-7.96 (m, 2H), 7.74 (br. s, 1H), 7.70 (d, J = 1.0 Hz, 1H), 7.51 (br. s, 1H), 7.46 (d, J = 7.8 Hz, 1H), 7.18 (d, J = 7.4 Hz, 1H), 7.14 (td, J = 7.6, 1.2 Hz, 1H), 6.97 (td, J = 7.4, 1.2 Hz, 1H), 2.18 (s, 3H). | 455.9 | D | |
| I- 768 | 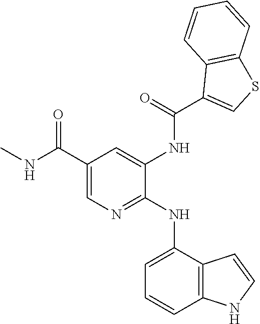 [SMILES: CNC(=O)c1cnc(Nc2cccc3[nH]ccc23)c(NC(=O)c2csc3ccccc23)c1] | (400 MHz, dmso) 11.12 (s, 1H), 10.51 (br s, 0.4H), 8.82 (s, 1H), 8.57-8.51 (m, 3H), 8.49 (br s, 2H), 8.40 (q, J = 4.0 Hz, 2H), 8.19 (d, J = 2.2 Hz, 1H), 8.11 (dd, J = 7.2, 1.2 Hz, 1H), 7.60-7.38 (m, 3H), 7.29-7.17 (m, 1H), 7.11 (d, J = 8.1 Hz, 1H), 7.04 (t, J = 7.8 Hz, 1H), 6.46- 6.34 (m, 1H), 2.79 (d, J = 4.5 Hz, 3H). Contains 14.5% w/w of formic acid. | 442.5 | C | |
| I- 769 | 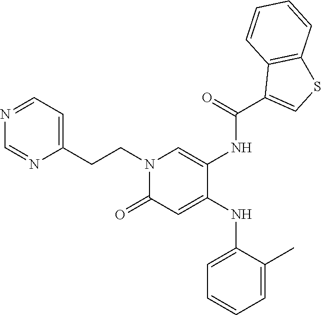 [SMILES: Cc1ccccc1Nc1cc(=O)n(CCc2ccncn2)cc1NC(=O)c1csc2ccccc12] | | | D | |
| I- 770 | 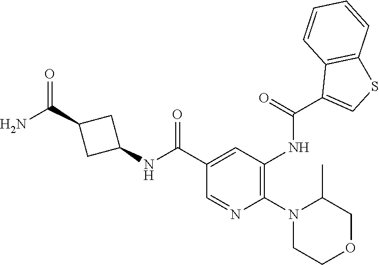 [SMILES: CC1COCCN1c1ncc(C(=O)N[C@H]2C[C@@H](C(N)=O)C2)cc1NC(=O)c1csc2ccccc12] | | | D | |
| I- 771 | 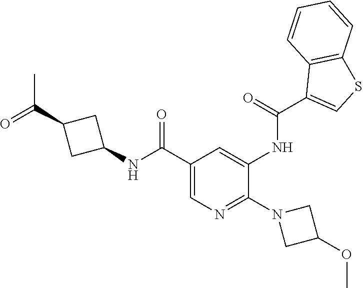 [SMILES: COC1CN(c2ncc(C(=O)N[C@H]3C[C@@H](C(C)=O)C3)cc2NC(=O)c2csc3ccccc23)C1] | | | D | |
| I- 772 | 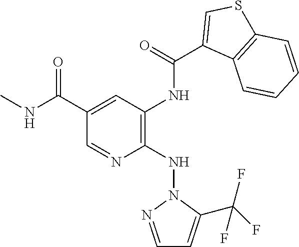 [SMILES: CNC(=O)c1cnc(Nn2nccc2C(F)(F)F)c(NC(=O)c2csc3ccccc23)c1] | | | A | B |
| I- 773 | 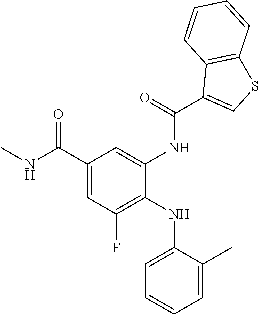 [SMILES: CNC(=O)c1cc(F)c(Nc2ccccc2C)c(NC(=O)c2csc3ccccc23)c1] | | | A | A |
| I- 774 | 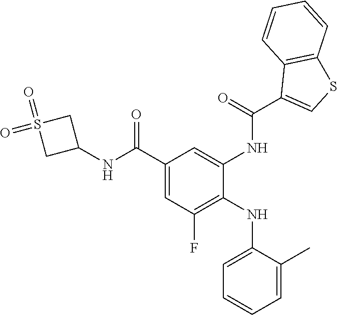 [SMILES: Cc1ccccc1Nc1c(F)cc(C(=O)NC2CS(=O)(=O)C2)cc1NC(=O)c1csc2ccccc12] | | | A | D |
| I- 775 | 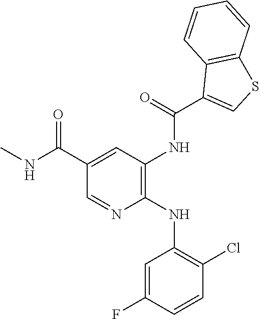 [SMILES: CNC(=O)c1cnc(Nc2cc(F)ccc2Cl)c(NC(=O)c2csc3ccccc23)c1] | (400 MHz, DMSO-d6) 2.80 (d, J = 4.39 Hz, 3 H) 6.89 (td, J = 8.42, 2.93 Hz, 1 H) 7.40-7.58 (m, 3 H) 8.10 (br d, J = 7.32 Hz, 1 H) 8.14-8.27 (m, 2 H) 8.39 (br d, J = 17.82 Hz, 1 H) 8.44- 8.54 (m, 1 H) 8.65 (br s, 1 H) 8.72 (s, 1 H) 10.51 (br s, 1 H) | 455.4 | D | |
| I- 776 | 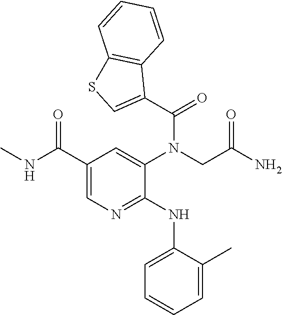 [SMILES: CNC(=O)c1cnc(Nc2ccccc2C)c(N(CC(N)=O)C(=O)c2csc3ccccc23)c1] | | | D | |
| I- 777 | 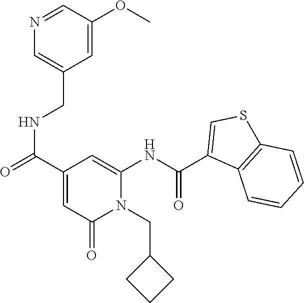 [SMILES: COc1cncc(CNC(=O)c2cc(NC(=O)c3csc4ccccc34)n(CC3CCC3)c(=O)c2)c1] | | | E | |
| I- 778 | 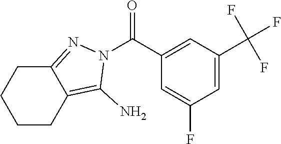 [SMILES: Nc1c2c(nn1C(=O)c1cc(F)cc(C(F)(F)F)c1)CCCC2] | | | E | |
| I- 779 | 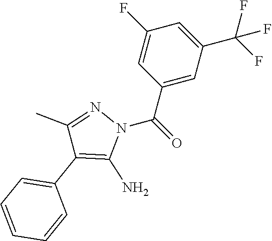 [SMILES: Cc1nn(C(=O)c2cc(F)cc(C(F)(F)F)c2)c(N)c1-c1ccccc1] | (400 MHz, DMSO-d6) 8.16-8.08 (m, 2H), 8.00 (dt, J = 8.7, 2.2 Hz, 1H), 7.45 (t, J = 7.6 Hz, 2H), 7.42-7.36 (m, 2H), 7.36-7.26 (m, 1H), 6.66 (d, J = 5.4 Hz, 2H), 2.11 (s, 3H). | 364.1 | D | |
| I- 780 | 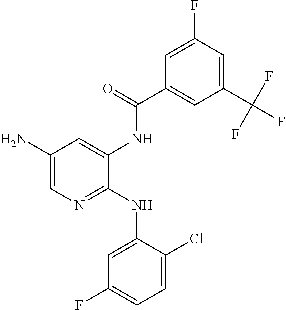 [SMILES: Nc1cnc(Nc2cc(F)ccc2Cl)c(NC(=O)c2cc(F)cc(C(F)(F)F)c2)c1] | (400 MHz, DMSO-d6) 10.58 (s, 1H), 8.18 (s, 1H), 8.11 (dt, J = 9.1, 2.0 Hz, 1H), 8.02 (dt, J = 8.7, 2.0 Hz, 1H), 7.74 (d, J = 2.6 Hz, 1H), 7.58-7.45 (m, 2H), 7.37 (dd, J = 8.8, 6.0 Hz, 1H), 7.14 (d, J = 2.6 Hz, 1H), 6.60 (td, J = 8.3, 3.0 Hz, 1H), 5.37 (s, 2H). | 443.05 | E | |
| I- 781 | 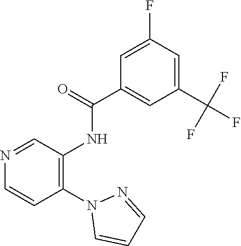 [SMILES: O=C(Nc1cnccc1-n1cccn1)c1cc(F)cc(C(F)(F)F)c1] | (400 MHz, Chloroform-d) 12.31 (s, 1H), 10.07 (s, 1H), 8.53 (d, J = 5.4 Hz, 1H), 8.17-8.11 (m, 2H), 7.98 (dd, J = 8.3, 2.0 Hz, 2H), 7.57 (dt, J = 8.2, 2.0 Hz, 1H), 7.41 (d, J = 5.4 Hz, 1H), 6.71- 6.65 (m, 1H). | 351.05 | D | |
| I- 782 | 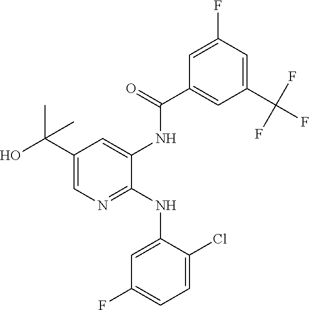 [SMILES: CC(C)(O)c1cnc(Nc2cc(F)ccc2Cl)c(NC(=O)c2cc(F)cc(C(F)(F)F)c2)c1] | (400 MHz, DMSO-d6) 10.76 (s, 1H), 8.37 (d, J = 2.3 Hz, 1H), 8.27 (s, 1H), 8.18 (dd, J = 9.2, 2.1 Hz, 1H), 8.12 (dd, J = 11.9, 3.1 Hz, 1H), 8.04 (dt, J = 8.7, 2.0 Hz, 1H), 7.91-7.84 (m, 2H), 7.47 (dd, J = 8.8, 6.0 Hz, 1H), 6.79 (ddd, J = 8.8, 7.9, 3.1 Hz, 1H), 5.24 (s, 1H), 1.49 (s, 6H) | 486 | D | |
| I- 783 | 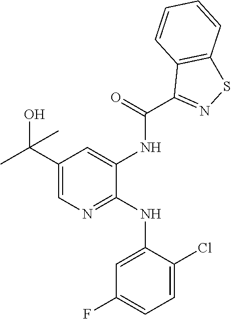 [SMILES: CC(C)(O)c1cnc(Nc2cc(F)ccc2Cl)c(NC(=O)c2nsc3ccccc23)c1] | (400 MHz, Methanol-d4) 8.91 (d, J = 8.1 Hz, 1H), 8.43 (d, J = 2.4 Hz, 1H), 8.20 (d, J = 8.2 Hz, 1H), 8.09-8.01 (m, 2H), 7.66 (dt, J = 26.3, 7.3 Hz, 2H), 7.36 (dd, J = 8.8, 5.9 Hz, 1H), 6.66 (td, J = 8.3, 2.9 Hz, 1H), 1.64 (s, 6H). | 457.1 | B | C |
| I- 784 | 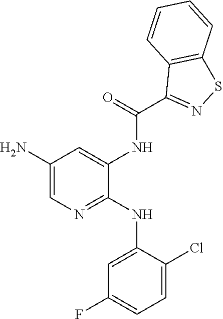 [SMILES: Nc1cnc(Nc2cc(F)ccc2Cl)c(NC(=O)c2nsc3ccccc23)c1] | (400 MHz, DMSO-d6) 10.64 (s, 1H), 8.84-8.76 (m, 1H), 8.37 (dt, J = 8.2, 1.0 Hz, 1H), 7.76-7.60 (m, 4H), 7.43 (d, J = 2.7 Hz, 1H), 7.36 (dd, J = 8.8, 6.0 Hz, 1H), 7.21 (dd, J = 11.9, 3.0 Hz, 1H), 6.59 (td, J = 8.4, 3.0 Hz, 1H), 5.38 (s, 2H) | 414 | E | |
| I- 785 | 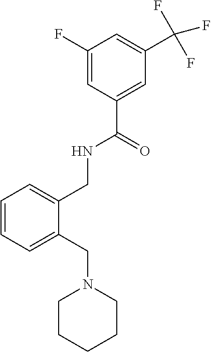 [SMILES: O=C(NCc1ccccc1CN1CCCCC1)c1cc(F)cc(C(F)(F)F)c1] | | | E | |
| I- 786 | 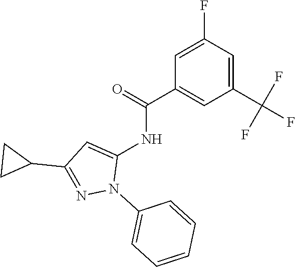 [SMILES: O=C(Nc1cc(C2CC2)nn1-c1ccccc1)c1cc(F)cc(C(F)(F)F)c1] | | | E | |
| I- 787 | 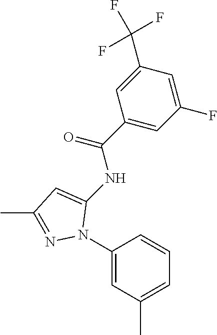 [SMILES: Cc1cccc(-n2nc(C)cc2NC(=O)c2cc(F)cc(C(F)(F)F)c2)c1] | | | E | |
| I- 788 | 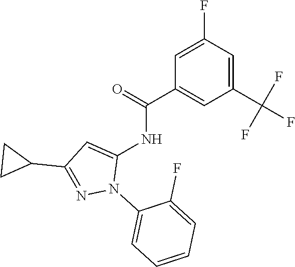 [SMILES: O=C(Nc1cc(C2CC2)nn1-c1ccccc1F)c1cc(F)cc(C(F)(F)F)c1] | | | E | |
| I- 789 | 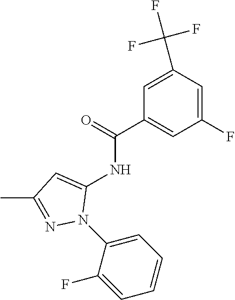 [SMILES: Cc1cc(NC(=O)c2cc(F)cc(C(F)(F)F)c2)n(-c2ccccc2F)n1] | | | D | |
| I- 790 | 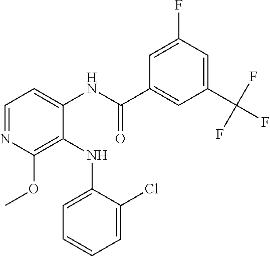 [SMILES: COc1nccc(NC(=O)c2cc(F)cc(C(F)(F)F)c2)c1Nc1ccccc1Cl] | (400 MHz, CD3CN) 8.91 (br. s, 1H), 8.08 (d, J = 5.7 Hz, 1H), 7.84 (d, J = 5.7 Hz, 1H), 7.71 (s, 1H), 7.65 (t, J = 8.1 Hz, 2H), 7.36 (dd, J = 7.9, 1.2 Hz, 1H), 7.11-7.00 (m, 1H), 6.77 (td, J = 7.9, 1.2 Hz, 1H), 6.32 (dd, J = 8.1, 1.1 Hz, 1H), 6.09 (s, 1H), 3.87 (s, 3H). | 440.3 | D | |
| I- 791 | 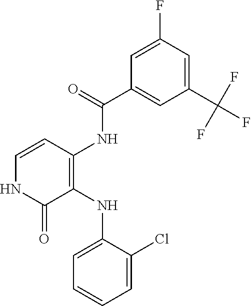 [SMILES: O=C(Nc1cc[nH]c(=O)c1Nc1ccccc1Cl)c1cc(F)cc(C(F)(F)F)c1] | (400 MHz, CD3CN) 8.66 (br. s, 1H), 7.67 (s, 1H), 7.62 (d, J = 8.6 Hz, 2H), 7.35 (dd, J = 7.9, 1.4 Hz, 1H), 7.23 (d, J = 7.4 Hz, 1H), 7.15 (d, J = 7.4 Hz, 1H), 7.11-7.03 (m, 1H), 6.81-6.73 (m, 1H), 6.48 (dd, J = 8.2, 1.4 Hz, 1H), 6.15 (s, 1H). | 426.3 | | |
| I- 792 | 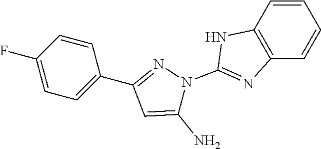 [SMILES: Nc1cc(-c2ccc(F)cc2)nn1-c1nc2ccccc2[nH]1] | (400 MHz, dmso) 12.69 (s, 1H), 7.99- 7.90 (m, 2H), 7.57 (s, 1H), 7.48 (s, 1H), 7.34-7.26 (m, 2H), 7.23-7.16 (m, 2H), 7.00 (s, 2H), 5.91 (s, 1H). | 294.1 | D | |
| I- 793 | 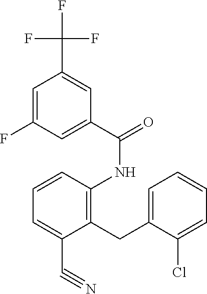 [SMILES: N#Cc1cccc(NC(=O)c2cc(F)cc(C(F)(F)F)c2)c1Cc1ccccc1Cl] | (400 MHz, DMSO-d6) 10.43 (s, 1H), 7.95 (d, J = 8.3 Hz, 1H), 7.87 (dd, J = 7.7, 0.9 Hz, 1H), 7.86-7.81 (m, J = 7.9 Hz, 2H), 7.76 (d, J = 8.0 Hz, 1H), 7.60 (t, J = 7.9 Hz, 1H), 7.37 (dd, J = 7.9, 1.0 Hz, 1H), 7.18 (td, J = 7.7, 1.3 Hz, 1H), 7.11 (dd, J = 10.8, 4.3 Hz, 1H), 6.69 (dd, J = 7.7, 1.0 Hz, 1H), 4.35 (d, J = 7.4 Hz, 2H). | 433.2 | B | |
| I- 794 | 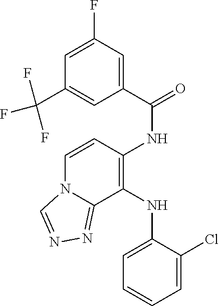 [SMILES: O=C(Nc1ccn2cnnc2c1Nc1ccccc1Cl)c1cc(F)cc(C(F)(F)F)c1] | (400 MHz, DMSO-d6) 9.03 (br s, 1H), 8.51 (d, J = 7.4 Hz, 1H), 8.18 (s, 1H), 7.84-7.82 (m, 1H), 7.77-7.72 (m, 1H), 7.66-7.62 (m, 1H), 7.60 (d, J = 7.4 Hz, 1H), 7.33 (dd, J = 8.0, 1.4 Hz, 1H), 6.98 (td, J = 4.1, 2.1 Hz, 1H), 6.77 (ddd, J = 8.9, 7.7, 1.5 Hz, 2H), 6.46 (dd, J = 8.2, 1.4 Hz, 1H). | 450.2 | E | |
| I- 795 | 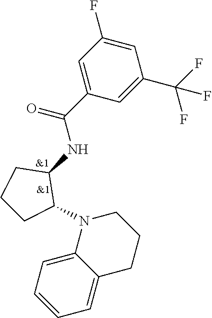 [SMILES: O=C(N[C@@H]1CCC[C@H]1N1CCCc2ccccc21)c1cc(F)cc(C(F)(F)F)c1] | (Chloroform-d, 400 MHz) 1.54-1.76 (2H, m), 1.87 (2H, dddd, J = 18.1, 9.9, 8.1, 4.2 Hz), 2.05 (1H, ddt, J = 12.7, 7.9, 4.3 Hz), 2.34 (1H, dtd, J = 12.2, 7.9, 4.0 Hz), 3.27 (1H, ddd, J = 11.9, 7.5, 3.0 Hz), 3.47 (1H, ddd, J = 11.9, 4.6, 2.8 Hz), 3.95-4.43 (3H, m), 4.58 (1H, tt, J = 10.0, 7.7 Hz), 6.10 (1H, d, J = 8.0 Hz), 6.65 (1H, ddd, J = 8.2, 6.5, 2.2 Hz), 6.77-6.93 (3H, m), 7.46 (2H, ddt, J = 18.8, 8.1, 2.1 Hz), 7.62 (1H, s), | 409.1 | E | |
| I- 796 | 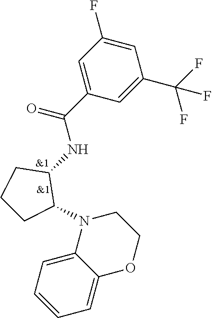 [SMILES: O=C(N[C@H]1CCC[C@H]1N1CCOc2ccccc21)c1cc(F)cc(C(F)(F)F)c1] | (Chloroform-d, 400 MHz) 1.60-1.80 (2H, m), 1.88-2.06 (2H, m), 2.16 (1H, dtd, J = 12.2, 7.8, 3.9 Hz), 2.46 (1H, s), 3.40 (1H, d, J = 12.2 Hz), 3.53 (1H, t, J = 8.2 Hz), 4.13-4.21 (1H, m), 4.31 (2H, dd, J = 9.8, 5.7 Hz), 4.49-4.59 (1H, m), 6.54 (1H, s), 6.74 (1H, d, J = 7.5 Hz), 6.84 (3H, t, J = 9.0 Hz), 7.37 (2H, dd, J = 19.2, 8.3 Hz), 7.48 (1H, s) | 409.15 | C | |
| I- 797 | 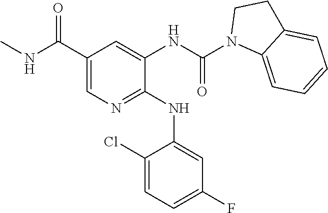 [SMILES: CNC(=O)c1cnc(Nc2cc(F)ccc2Cl)c(NC(=O)N2CCc3ccccc32)c1] | (DMSO-d6, 400 MHz) 2.81 (3H, d), 3.24 (2H, t), 4.20 (2H, t), 6.88 (1H, ddd), 6.94 (1H, td), 7.10-7.19 (1H, m), 7.24 (1H, d), 7.52 (1H, dd), 7.89 (1H, d), 8.10 (1H, d), 8.17-8.28 (1H, m), 8.40-8.50 (2H, m), 8.65 (1H, d), 8.74 (1H, s) | 440.1 | E | |
| I- 798 | 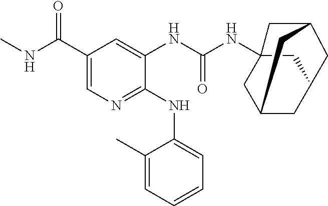 [SMILES: CNC(=O)c1cnc(Nc2ccccc2C)c(NC(=O)NC23C[C@H]4C[C@@H](C2)C[C@@H](C3)C4)c1] | (DMSO-d6, 400 MHz) 8.29-8.23 (2H, m), 8.08 (1H, d, J = 2.2 Hz), 7.92 (2H, d, J = 5.9 Hz), 7.53 (1H, dd, J = 8.0, 1.3 Hz), 7.25-7.10 (2H, m), 6.98 (1H, td, J = 7.4, 1.3 Hz), 6.23 (1H, s), 2.75 (3H, d, J = 4.5 Hz), 2.14 (3H, s), 2.05 (3H, s), 1.97 (6H, d, J = 2.9 Hz), 1.64 (6H, t, J = 3.0 Hz) | 434.25 | D | |
| I- 799 | 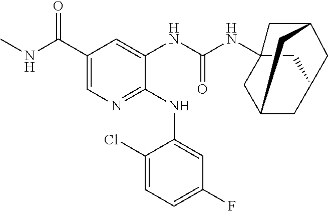 [SMILES: CNC(=O)c1cnc(Nc2cc(F)ccc2Cl)c(NC(=O)NC23C[C@H]4C[C@@H](C2)C[C@@H](C3)C4)c1] | (DMSO-d6, 400 MHz) 8.51 (2H, dd, J = 16.9, 1.8 Hz), 8.42 (1H, q, J = 4.4 Hz), 8.13 (1H, s), 8.08-7.99 (2H, m), 7.49 (1H, dd, J = 8.9, 6.0 Hz), 6.84 (1H, ddd, J = 8.8, 7.9, 3.0 Hz), 6.35 (1H, s), 2.78 (3H, d, J = 4.5 Hz), 2.04 (3H, s), 1.97 (6H, d, J = 2.9 Hz), 1.64 (6H, d, J = 3.0 Hz) | 472.25 | E | |
| I- 801 | 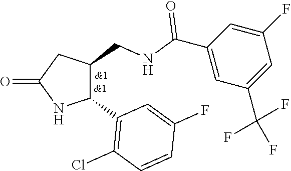 [SMILES: O=C1C[C@@H](CNC(=O)c2cc(F)cc(C(F)(F)F)c2)[C@H](c2cc(F)ccc2Cl)N1] | (DMSO-d6, 400 MHz) 9.04 (1H, t, J = 5.9 Hz), 8.17 (1H, s), 8.02 (1H, s), 7.92 (2H, td, J = 9.0, 8.3, 2.2 Hz), 7.49 (1H, dd, J = 8.8, 5.1 Hz), 7.19 (1H, td, J = 8.4, 3.1 Hz), 7.11 (1H, dd, J = 9.7, 3.1 Hz), 4.79 (1H, s), 3.55 (1H, dt, J = 13.0, 6.3 Hz), 3.45 (1H, dt, J = 13.1, 6.0 Hz), 2.50-2.43 (2H, m), 2.12-2.01 (1H, m) | 433 | B | B |
| I- 802 | 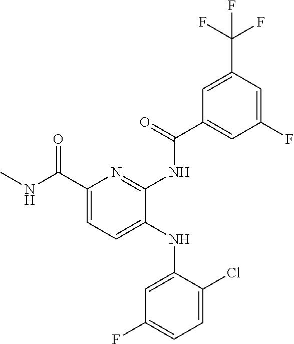 [SMILES: CNC(=O)c1ccc(Nc2cc(F)ccc2Cl)c(NC(=O)c2cc(F)cc(C(F)(F)F)c2)n1] | (Chloroform-d, 400 MHz) 8.64 (1H, s), 8.14-8.05 (2H, m), 8.00-7.93 (1H, m), 7.89 (1H, d, J = 8.4 Hz), 7.71 (1H, s), 7.61 (2H, d, J = 7.6 Hz), 7.35 (1H, dd, J = 8.8, 5.7 Hz), 6.81 (1H, dd, J = 10.2, 2.8 Hz), 6.64 (1H, ddd, J = 8.8, 7.7, 2.8 Hz), 3.03 (3H, d, J = 5.0 Hz) | 485.25 | B | B |
| I- 803 | 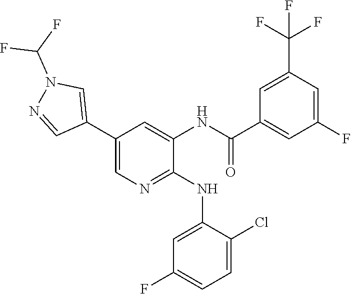 [SMILES: O=C(Nc1cc(-c2cnn(C(F)F)c2)cnc1Nc1cc(F)ccc1Cl)c1cc(F)cc(C(F)(F)F)c1] | (DMSO-d6, 400 MHz) 10.77 (1H, s), 8.77 (1H, s), 8.66 (1H, d, J = 2.3 Hz), 8.33 (1H, s), 8.29 (1H, s), 8.22-8.08 (3H, m), 8.04 (2H, t, J = 8.5 Hz), 7.80 (1H, d, J = 59.2 Hz), 7.51 (1H, dd, J = 8.9, 6.0 Hz), 6.86 (1H, ddd, J = 8.9, 7.9, 3.1 Hz) | 544.15 | D | |
| I- 804 | 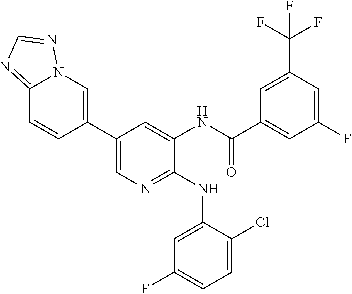 [SMILES: O=C(Nc1cc(-c2ccc3ncnn3c2)cnc1Nc1cc(F)ccc1Cl)c1cc(F)cc(C(F)(F)F)c1] | (DMSO-d6, 400 MHz) 10.78 (1H, s), 9.40 (1H, dd, J = 1.8, 0.9 Hz), 8.72 (1H, d, J = 2.4 Hz), 8.56 (1H, s), 8.34 (1H, d, J = 2.4 Hz), 8.30 (1H, s), 8.26-8.17 (2H, m), 8.15 (1H, dd, J = 11.5, 3.1 Hz), 8.06 (2H, ddd, J = 10.3, 8.1, 1.9 Hz), 7.98 (1H, dd, J = 9.3, 1.0 Hz), 7.54 (1H, dd, J = 8.9, 6.0 Hz), 6.91 (1H, ddd, J = 8.9, 7.9, 3.1 Hz) | 545.1 | E | |
| I- 805 | 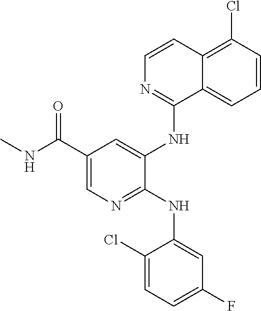 [SMILES: CNC(=O)c1cnc(Nc2cc(F)ccc2Cl)c(Nc2nccc3c(Cl)cccc23)c1] | (DMSO-d6, 400 MHz) 9.57 (1H, s), 8.70 (1H, d, J = 2.2 Hz), 8.51 (2H, dd, J = 17.6, 6.7 Hz), 8.30 (1H, dd, J = 11.6, 3.0 Hz), 8.23-8.12 (2H, m), 8.05 (1H, d, J = 6.1 Hz), 7.97 (1H, d, J = 7.5 Hz), 7.68 (1H, t, J = 8.0 Hz), 7.57-7.34 (2H, m), 6.83 (1H, td, J = 8.4, 3.0 Hz), 2.81 (3H, d, J = 4.5 Hz) | 456.05 | E | |
| I- 806 | 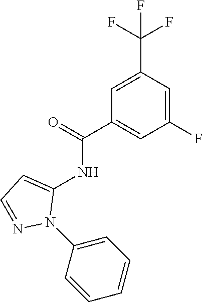 [SMILES: O=C(Nc1ccnn1-c1ccccc1)c1cc(F)cc(C(F)(F)F)c1] | | | E | |
| I- 807 | 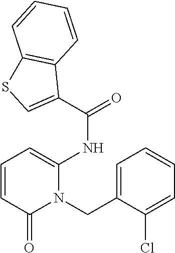 [SMILES: O=C(Nc1cccc(=O)n1Cc1ccccc1Cl)c1csc2ccccc12] | | | E | |
| I- 808 | 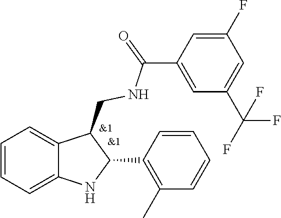 [SMILES: Cc1ccccc1[C@@H]1Nc2ccccc2[C@H]1CNC(=O)c1cc(F)cc(C(F)(F)F)c1] | (400 MHz, Chloroform-d) 7.59 (s, 1H), 7.47 (dd, J = 17.1, 8.3 Hz, 2H), 7.31 (d, J = 7.4 Hz, 1H), 7.24-7.11 (m, 5H), 6.87-6.78 (m, 2H), 6.31 (s, 1H), 4.97 (d, J = 4.7 Hz, 1H), 4.02 (dt, J = 13.5, 5.4 Hz, 1H), 2.47 (s, 3H), 3.83 (dt, J = 13.5, 5.5 Hz, 1H), 3.59 (q, J = 5.2 Hz, 1H). | 429.2 | E | |
| I- 809 | 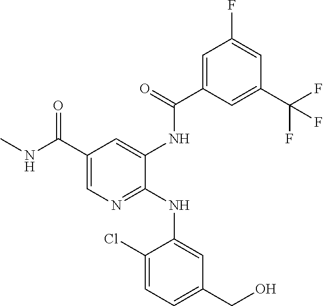 [SMILES: CNC(=O)c1cnc(Nc2cc(CO)ccc2Cl)c(NC(=O)c2cc(F)cc(C(F)(F)F)c2)c1] | (DMSO-d6, 400 MHz) 10.49 (1H, s), 8.54 (1H, t, J = 2.6 Hz), 8.43-8.32 (2H, m), 8.26 (1H, s), 8.19 (1H, dt, J = 9.2, 2.0 Hz), 8.10 (1H, d, J = 2.2 Hz), 8.02 (1H, dt, J = 8.6, 2.0 Hz), 7.79 (1H, dd, J = 11.3, 2.0 Hz), 7.43 (1H, d, J = 8.2 Hz), 7.08 (1H, dd, J = 8.2, 2.0 Hz), 5.30 (1H, t, J = 5.7 Hz), 4.50 (2H, d, J = 5.8 Hz), 2.79 (3H, d, J = 4.3 Hz) | 497.05 | B | B |
| I- 810 | 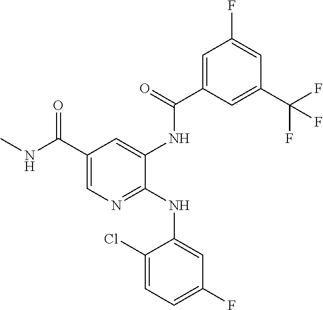 [SMILES: CNC(=O)c1cnc(Nc2cc(F)ccc2Cl)c(NC(=O)c2cc(F)cc(C(F)(F)F)c2)c1] | (DMSO-d6, 400 MHz) 10.67 (1H, d, J = 2.4 Hz), 8.66 (1H, t, J = 2.3 Hz), 8.48 (1H, d, J = 4.7 Hz), 8.29 (1H, s), 8.27 (1H, s), 8.22-8.14 (2H, m), 8.08-7.95 (2H, m), 7.54 (1H, dd, J = 8.9, 5.9 Hz), 6.96 (1H, td, J = 8.4, 3.0 Hz), 2.81 (3H, d, J = 4.2 Hz) | 485.05 | E | |
| I- 811 | 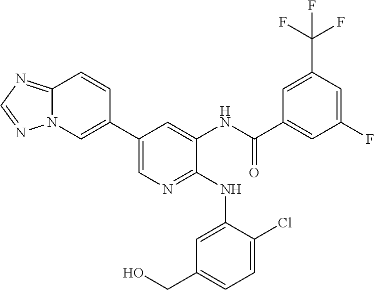 [SMILES: O=C(Nc1cc(-c2ccc3ncnn3c2)cnc1Nc1cc(CO)ccc1Cl)c1cc(F)cc(C(F)(F)F)c1] | (DMSO-d6, 400 MHz) 10.62 (1H, d, J = 3.0 Hz), 9.36 (1H, s), 8.85-8.45 (2H, m), 8.32-8.12 (4H, m), 8.06 (2H, td, J = 6.5, 3.3 Hz), 7.99-7.88 (2H, m), 7.43 (1H, d, J = 8.2 Hz), 7.05 (1H, dd, J = 8.2, 2.0 Hz), 5.30 (1H, t, J = 5.7 Hz), 4.50 (2H, d, J = 5.8 Hz) | 557.1 | E | |
| I- 812 | 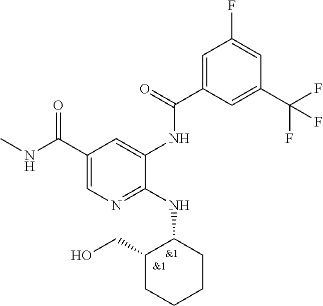 [SMILES: CNC(=O)c1cnc(N[C@@H]2CCCC[C@@H]2CO)c(NC(=O)c2cc(F)cc(C(F)(F)F)c2)c1] | (DMSO-d6, 400 MHz) 10.23 (1H, s), 8.49 (1H, d, J = 2.2 Hz), 8.21 (1H, q, J = 5.2, 4.8 Hz), 8.17 (1H, s), 8.11 (1H, dd, J = 9.0, 2.2 Hz), 8.00 (1H, dt, J = 8.6, 2.0 Hz), 7.87 (1H, d, J = 2.2 Hz), 6.46 (1H, d, J = 7.3 Hz), 4.69 (1H, t, J = 5.1 Hz), 4.28 (1H, dq, J = 7.5, 3.6 Hz), 3.57 (1H, ddd, J = 11.1, 8.0, 5.1 Hz), 3.37 (1H, d, J = 5.7 Hz), 2.76 (3H, d, J = 4.4 Hz), 1.91 (1H, q, J = 7.9, 6.0 Hz), 1.79- 1.63 (2H, m), 1.59 (1H, d, J = 17.0 Hz), 1.52-1.22 (5H, m) | 469.2 | E | |
| I- 813 | 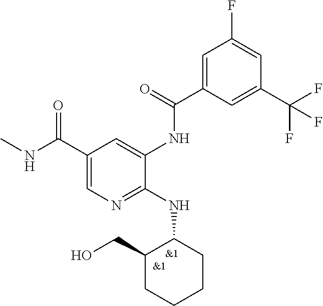 [SMILES: CNC(=O)c1cnc(N[C@@H]2CCCC[C@H]2CO)c(NC(=O)c2cc(F)cc(C(F)(F)F)c2)c1] | (DMSO-d6, 400 MHz) 9.98 (1H, s), 8.44 (1H, d, J = 2.2 Hz), 8.26-8.11 (3H, m), 8.00 (1H, dt, J = 8.5, 2.1 Hz), 7.83 (1H, d, J = 2.3 Hz), 6.50 (1H, d, J = 8.3 Hz), 4.36 (1H, t, J = 5.3 Hz), 3.89 (1H, dtd, J = 11.0, 7.6, 3.8 Hz), 3.44 (1H, ddd, J = 10.2, 6.1, 3.4 Hz), 3.26 (1H, dt, J = 10.8, 5.7 Hz), 2.75 (3H, d, J = 4.4 Hz), 1.89 (2H, t, J = 10.6 Hz), 1.74-1.66 (2H, m), 1.55-1.45 (1H, m), 1.31 (2H, dd, J = 13.9, 5.3 Hz), 1.18 (2H, q, J = 11.2, 9.9 Hz) | 469.15 | | |
| I- 814 | 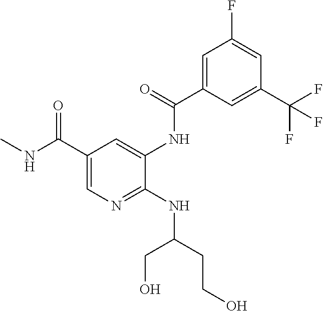 [SMILES: CNC(=O)c1cnc(NC(CO)CCO)c(NC(=O)c2cc(F)cc(C(F)(F)F)c2)c1] | (DMSO-d6, 400 MHz) 10.01 (1H, s), 8.46 (1H, d, J = 2.2 Hz), 8.19 (2H, d, J = 2.8 Hz), 8.18-8.11 (1H, m), 8.00 (1H, d, J = 8.4 Hz), 7.88 (1H, d, J = 2.2 Hz), 6.39 (1H, d, J = 8.0 Hz), 4.68 (1H, t, J = 5.5 Hz), 4.48 (1H, t, J = 5.1 Hz), 4.28 (1H, q, J = 7.9, 7.4 Hz), 3.50 (3H, dq, J = 13.1, 6.9, 6.1 Hz), 3.40 (1H, dt, J = 11.2, 5.9 Hz), 2.76 (3H, d, J = 4.4 Hz), 1.89-1.77 (1H, m), 1.66 (1H, ddt, J = 14.0, 8.4, 5.9 Hz) | 445.05 | E | |
| I- 815 | 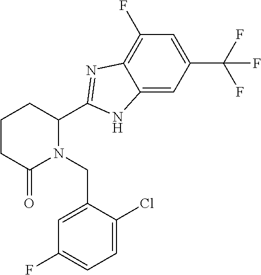 [SMILES: O=C1CCCC(c2nc3c(F)cc(C(F)(F)F)cc3[nH]2)N1Cc1cc(F)ccc1Cl] | (DMSO-d6, 400 MHz) 7.71 (1H, s), 7.35 (1H, dd, J = 10.8, 1.5 Hz), 7.27 (2H, ddt, J = 8.9, 5.9, 2.8 Hz), 7.12 (1H, t, J = 9.1 Hz), 5.02-4.92 (2H, m), 4.00 (1H, d, J = 15.7 Hz), 2.47 (2H, t, J = 4.5 Hz), 2.24 (1H, dddd, J = 14.1, 11.2, 5.7, 3.7 Hz), 2.13 (1H, dq, J = 13.6, 4.3 Hz), 1.86-1.64 (2H, m) | 444.1 | E | |
| I- 816 | 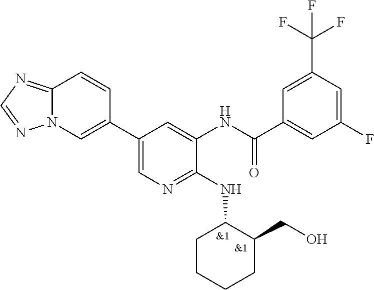 [SMILES: O=C(Nc1cc(-c2ccc3ncnn3c2)cnc1N[C@H]1CCCC[C@@H]1CO)c1cc(F)cc(C(F)(F)F)c1] | (Methanol-d4, 400 MHz) 9.08-9.02 (1H, m), 8.43 (1H, d, J = 2.7 Hz), 8.37 (1H, dd, J = 12.9, 2.3 Hz), 8.23 (1H, d, J = 13.6 Hz), 8.08 (1H, t, J = 9.3 Hz), 8.01 (1H, dd, J = 9.3, 1.9 Hz), 7.92 (1H, t, J = 2.3 Hz), 7.89-7.82 (1H, m), 7.77 (1H, t, J = 8.4 Hz), 4.59 (1H, s), 3.93 (1H, s), 3.67-3.56 (1H, m), 3.56-3.43 (1H, m), 2.13-1.94 (1H, m), 1.93-1.72 (3H, m), 1.69-1.25 (6H, m) | 539.2 | E | |
| I- 817 | 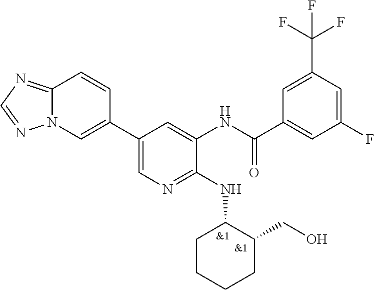 [SMILES: O=C(Nc1cc(-c2ccc3ncnn3c2)cnc1N[C@H]1CCCC[C@H]1CO)c1cc(F)cc(C(F)(F)F)c1] | (DMSO-d6, 400 MHz) 10.05 (1H, s), 9.23 (1H, t, J = 1.2 Hz), 8.50 (1H, s), 8.44 (1H, d, J = 2.4 Hz), 8.25-8.15 (2H, m), 8.06-7.87 (4H, m), 6.32 (1H, d, J = 8.2 Hz), 4.39 (1H, s), 3.93-3.86 (1H, m), 3.49 (1H, dd, J = 10.6, 3.5 Hz), 3.29 (1H, d, J = 6.3 Hz), 2.00-1.93 (1H, m), 1.89 (1H, d, J = 8.4 Hz), 1.72 (2H, t, J = 8.4 Hz), 1.61-1.44 (1H, m), 1.32 (2H, t, J = 8.9 Hz), 1.28-1.13 (2H, m) | 529.2 | E | |
| I- 818 | 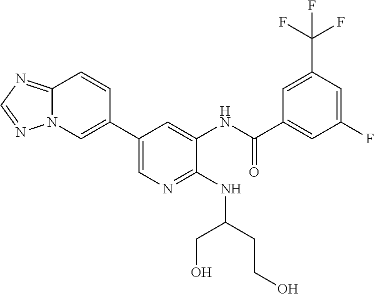 [SMILES: O=C(Nc1cc(-c2ccc3ncnn3c2)cnc1NC(CO)CCO)c1cc(F)cc(C(F)(F)F)c1] | (DMSO-d6, 400 MHz) 10.09 (1H, s), 9.25 (1H, dd, J = 1.8, 0.9 Hz), 8.51 (1H, s), 8.45 (1H, d, J = 2.4 Hz), 8.23 (1H, s), 8.18 (1H, dd, J = 9.3, 2.5 Hz), 8.06-7.95 (3H, m), 7.91 (1H, dd, J = 9.3, 0.9 Hz), 6.21 (1H, d, J = 8.0 Hz), 4.70 (1H, t, J = 5.6 Hz), 4.51 (1H, t, J = 5.1 Hz), 4.28 (1H, q, J = 7.5 Hz), 3.60-3.48 (3H, m), 3.48-3.38 (1H, m), 1.86 (1H, dtd, J = 12.3, 7.3, 4.9 Hz), 1.69 (1H, ddt, J = 14.1, 8.5, 6.0 Hz) | 505.15 | E | |
| I- 819 | 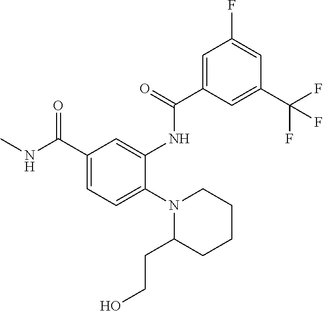 [SMILES: CNC(=O)c1ccc(N2CCCCC2CCO)c(NC(=O)c2cc(F)cc(C(F)(F)F)c2)c1] | (Chloroform-d, 400 MHz) 9.43 (1H, s), 8.88 (1H, d, J = 2.3 Hz), 8.62 (1H, d, J = 2.3 Hz), 7.96 (1H, s), 7.84 (1H, dt, J = 8.5, 2.0 Hz), 7.57 (1H, dt, J = 7.9, 2.1 Hz), 6.45 (1H, d, J = 5.1 Hz), 3.69 (1H, s), 3.67-3.54 (2H, m), 3.13-2.93 (5H, m), 2.25-2.65 (1H, m), 2.07-1.97 (1H, m), 1.89 (1H, d, J = 10.9 Hz), 1.84-1.77 (1H, m), 1.77-1.71 (1H, m), 1.67 (1H, s), 1.60 (3H, ddd, J = 14.1, 8.5, 5.5 Hz) | 469.1 | D | |
| I- 820 | 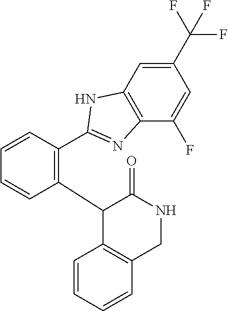 [SMILES: O=C1NCc2ccccc2C1c1ccccc1-c1nc2c(F)cc(C(F)(F)F)cc2[nH]1] | (Chloroform-d, 400 MHz) 14.48 (1H, s), 8.17 (1H, d, J = 7.7 Hz), 7.61-7.89 (1H, m), 7.44 (1H, t, J = 7.5 Hz), 7.38 (2H, d, J = 8.0 Hz), 7.34 (2H, dd, J = 8.9, 1.9 Hz), 7.24 (1H, t, J = 7.0 Hz), 6.91- 6.83 (2H, m), 6.58 (1H, s), 5.29 (1H, s), 5.08 (1H, d, J = 16.3 Hz), 4.79-4.70 (1H, m) | 426.1 | E | |
| I- 821 | 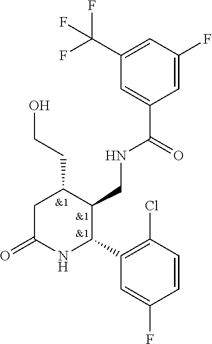 [SMILES: O=C1C[C@H](CCO)[C@@H](CNC(=O)c2cc(F)cc(C(F)(F)F)c2)[C@H](c2cc(F)ccc2Cl)N1] | (DMSO-d6, 400 MHz) 8.63-8.55 (1H, m), 7.90 (2H, d, J = 14.1 Hz), 7.86-7.76 (2H, m), 7.46 (1H, ddd, J = 29.6, 8.8, 5.2 Hz), 7.30 (1H, ddd, J = 31.3, 9.8, 3.1 Hz), 7.15 (1H, qd, J = 8.4, 3.0 Hz), 4.91 (1H, dd, J = 115.9, 6.0 Hz), 4.47 (1H, dt, J = 29.1, 5.0 Hz), 3.53 (1H, q, J = 6.1 Hz), 3.46-3.36 (1H, m), 3.32-3.19 (1H, m), 2.88-2.80 (1H, m), 2.67 (1H, dd, J = 17.8, 6.1 Hz), 2.46 (1H, d, J = 11.5 Hz), 2.28-2.00 (2H, m), 1.71-1.50 (2H, m) | 491.1 | E | |
| I- 822 | 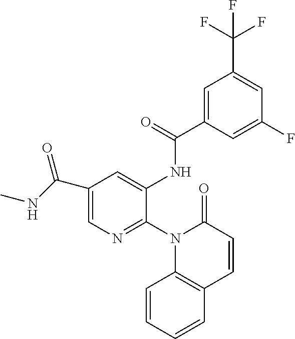 [SMILES: CNC(=O)c1cnc(-n2c(=O)ccc3ccccc32)c(NC(=O)c2cc(F)cc(C(F)(F)F)c2)c1] | (400 MHz, Chloroform-d) 9.12 (s, 1H), 8.94 (d, J = 2.2 Hz, 1H), 8.79 (d, J = 2.2 Hz, 1H), 7.96 (d, J = 9.5 Hz, 1H), 7.71 (s, 1H), 7.69-7.59 (m, 2H), 7.46 (d, J = 8.1 Hz, 1H), 7.41 (ddd, J = 8.7, 7.3, 1.6 Hz, 1H), 7.34-7.29 (m, 1H), 6.83 (s, 1H), 6.78 (d, J = 13.8 Hz, 1H), 6.61 (d, J = 8.5 Hz, 1H), 3.13 (d, J = 4.8 Hz, 3H) | 485.1 | E | |
| I- 823 | 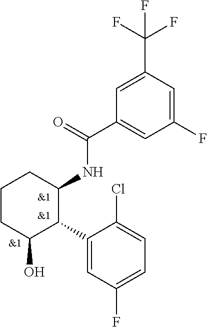 [SMILES: O=C(N[C@@H]1CCC[C@H](O)[C@H]1c1cc(F)ccc1Cl)c1cc(F)cc(C(F)(F)F)c1] | (Chloroform-d, 400 MHz) 7.55 (1H, s), 7.50-7.34 (3H, m), 7.21 (1H, dd, J = 9.4, 3.0 Hz), 6.93 (1H, ddd, J = 8.9, 7.4, 3.0 Hz), 6.12 (1H, d, J = 8.9 Hz), 4.16 (1H, d, J = 11.3 Hz), 3.97 (1H, s), 3.31 (1H, t, J = 10.8 Hz), 2.25 (2H, ddt, J = 11.7, 7.6, 3.8 Hz), 1.97 (1H, dp, J = 12.6, 3.1, 2.6 Hz), 1.66 (1H, dt, J = 13.2, 3.3 Hz), 1.61-1.51 (2H, m), 1.50-1.34 (1H, m) | 434 | E | |
| I- 824 | 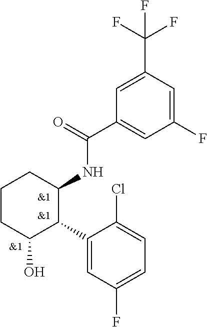 [SMILES: O=C(N[C@@H]1CCC[C@@H](O)[C@H]1c1cc(F)ccc1Cl)c1cc(F)cc(C(F)(F)F)c1] | (Chloroform-d, 400 MHz) 7.45 (2H, dd, J = 8.8, 5.0 Hz), 7.21 (1H, d, J = 8.2 Hz), 7.14-7.05 (1H, m), 6.84 (1H, dd, J = 8.7, 2.9 Hz), 5.65 (1H, s), 4.81 (1H, d, J = 16.8 Hz), 3.92 (1H, dd, J = 12.0, 3.8 Hz), 3.76 (1H, s), 3.61 (2H, d, J = 6.5 Hz), 3.06 (1H, d, J = 16.8 Hz), 2.89 (3H, d, J = 4.7 Hz), 2.26 (2H, d, J = 12.3 Hz), 2.11-1.93 (3H, m), 1.47 (1H, t, J = 13.4 Hz), 1.38-1.16 (4H, m) | 493.15 | E | |
| I- 825 | 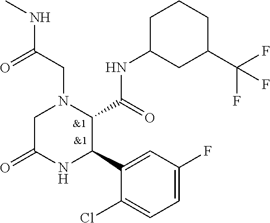 [SMILES: CNC(=O)CN1CC(=O)N[C@H](c2cc(F)ccc2Cl)[C@H]1C(=O)NC1CCCC(C(F)(F)F)C1] | (Chloroform-d, 400 MHz) 7.41 (1H, ddd, J = 8.7, 5.0, 1.3 Hz), 7.20-7.01 (2H, m), 6.90 (1H, d, J = 8.5 Hz), 6.20 (1H, s), 5.84 (1H, s), 4.48-3.79 (1H, m), 3.77-3.46 (3H, m), 2.22 (2H, s), 1.99 (3H, dd, J = 28.1, 9.1 Hz), 1.43 (2H, tt, J = 12.5, 7.9 Hz), 1.33-1.00 (3H, m) | 422.05 | E | |
| I- 826 | 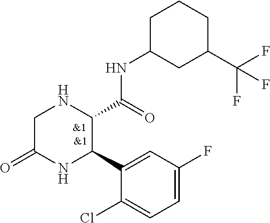 [SMILES: O=C1CN[C@H](C(=O)NC2CCCC(C(F)(F)F)C2)[C@@H](c2cc(F)ccc2Cl)N1] | (DMSO-d6, 400 MHz) 10.30 (1H, s), 8.50 (1H, d, J = 4.9 Hz), 8.10 (1H, s), 8.00-7.92 (3H, m), 7.82 (1H, s), 7.41 (1H, dd, J = 8.8, 5.8 Hz), 6.72 (1H, dd, J = 11.1, 2.9 Hz), 6.66 (1H, td, J = 8.3, 2.9 Hz), 2.77 (3H, d, J = 4.5 Hz) | 490.05 | B | |
| I- 827 | 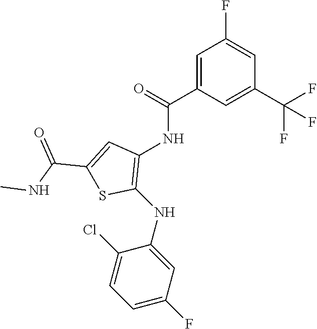 [SMILES: CNC(=O)c1cc(NC(=O)c2cc(F)cc(C(F)(F)F)c2)c(Nc2cc(F)ccc2Cl)s1] | (DMSO-d6, 400 MHz) 8.57-8.53 (1H, m), 8.11 (1H, s), 8.05 (1H, s), 7.93 (2H, dd, J = 16.1, 8.0 Hz), 7.80-7.73 (1H, m), 7.59-7.46 (2H, m), 7.39 (1H, d, J = 7.3 Hz), 7.33-7.19 (4H, m), 7.02 (2H, dtd, J = 18.9, 7.4, 1.2 Hz), 5.73 (1H, d, J = 6.0 Hz), 5.29 (1H, ddd, J = 9.0, 6.0, 3.3 Hz), 4.35 (1H, dd, J = 11.1, 7.9 Hz), 4.04 (1H, dd, J = 11.1, 3.4 Hz), 2.25 (3H, s). | 411.3 | C | |
| I- 828 | 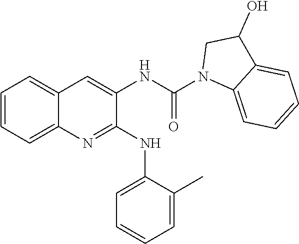 [SMILES: Cc1ccccc1Nc1nc2ccccc2cc1NC(=O)N1CC(O)c2ccccc21] | (DMSO-d6, 400 MHz) 10.29 (1H, s), 8.43 (1H, d, J = 2.2 Hz), 8.29 (2H, s), 8.23 (1H, s), 8.18 (1H, d, J = 9.1 Hz), 8.03-7.96 (2H, m), 7.32 (1H, dd, J = 7.9, 1.4 Hz), 7.27-7.22 (1H, m), 7.19 (1H, td, J = 7.6, 1.7 Hz), 7.09 (1H, td, J = 7.4, 1.4 Hz), 2.77 (3H, d, J = 4.5 Hz), 2.16 (3H, s) | 447.05 | C | C |
| I- 829 | 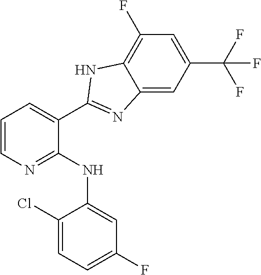 [SMILES: Fc1ccc(Cl)c(Nc2ncccc2-c2nc3cc(C(F)(F)F)cc(F)c3[nH]2)c1] | (400 MHz, DMSO-d6): 6.88 (1H, ddd), 7.20 (1H, dd), 7.45-7.67 (2H, m), 7.85 (1H, d), 8.44-8.70 (2H, m), 8.86 (1H, ddd), 12.35 (1H, d), 14.01 (1H, d). | 425.05 | | |

In chemical structures in Table 1, above, and the Examples, below, stereogenic centers are described according to the Enhanced Stereo Representation format (MDL/Biovia, e.g. using labels “or1”, “or2”, “abs”, “and1”). (See, for example, the structures of Compounds I-64, I-68, and I-165.)

In some embodiments, the present disclosure provides a compound in Table 1, above, wherein the compound is denoted as having an ADP-Glo IC₅₀ of “A”. In some embodiments, the present disclosure provides a compound in Table 1, above, wherein the compound is denoted as having an ADP-Glo IC₅₀ of “A” or “B”. In some embodiments, the present disclosure provides a compound in Table 1, above, wherein the compound is denoted as having an ADP-Glo IC₅₀ of “A” or “B” or “C”. In some embodiments, the present disclosure provides a compound in Table 1, above, wherein the compound is denoted as having an ADP-Glo IC₅₀ of “A” or “B” or “C” or “D”.

In some embodiments, the present disclosure provides a compound in Table 1, above, wherein the compound is denoted as having an MCF10A IC₅₀ of “A”. In some embodiments, the present disclosure provides a compound in Table 1, above, wherein the compound is denoted as having an MCF10A IC₅₀ of “A” or “B”. In some embodiments, the present disclosure provides a compound in Table 1, above, wherein the compound is denoted as having an MCF10A IC₅₀ of “A” or “B” or “C”. In some embodiments, the present disclosure provides a compound in Table 1, above, wherein the compound is denoted as having an MCF10A IC₅₀ of “A” or “B” or “C” or “D”.

In some embodiments, the present disclosure comprises a compound of formula I selected from those depicted in Table 1, above, or a pharmaceutically acceptable salt, stereoisomer, or mixture of stereoisomers thereof. In some embodiments, the present disclosure provides a compound of formula I selected from those depicted in Table 1, above, or a pharmaceutically acceptable salt thereof. In some embodiments, the present disclosure provides a compound of formula I selected from those depicted in Table 1, above.

In some embodiments, the present disclosure comprises a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, selected from those depicted in Table 1, above, or a pharmaceutically acceptable salt, stereoisomer, or mixture of stereoisomers thereof. In some embodiments, the present disclosure provides a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, selected from those depicted in Table 1, above, or a pharmaceutically acceptable salt thereof. In some embodiments, the present disclosure provides a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, LI, LII, LIII, LIV, LV, LVI, LVII, or LVIII, selected from those depicted in Table 1, above.

### 4. Uses, Formulation, and Administration

### Pharmaceutically Acceptable Compositions

According to another embodiment, the disclosure provides a composition comprising a compound of this disclosure, or a pharmaceutically acceptable derivative thereof, and a pharmaceutically acceptable carrier, adjuvant, or vehicle. In some embodiments, the disclosure provides a pharmaceutical composition comprising a compound of this disclosure, and a pharmaceutically acceptable carrier. The amount of compound in compositions of this disclosure is such that is effective to measurably inhibit a PI3Kα protein kinase, or a mutant thereof, in a biological sample or in a patient. In certain embodiments, the amount of compound in compositions of this disclosure is such that it is effective to measurably inhibit a PI3Kα protein kinase, or a mutant thereof, in a biological sample or in a patient. In certain embodiments, a composition of this disclosure is formulated for administration to a patient in need of such composition. In some embodiments, a composition of this disclosure is formulated for oral administration to a patient.

The terms “subject” and “patient,” as used herein, means an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “inhibitorily active metabolite or residue thereof” means that a metabolite or residue thereof is also an inhibitor of a PI3Kα protein kinase, or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the patient treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

The precise dose to be employed in the compositions will also depend on the route of administration, and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range, and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Ale^{YA}nder T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Uses of Compounds and Pharmaceutically Acceptable Compositions

Compounds and compositions described herein are generally useful for the inhibition of a kinase or a mutant thereof. In some embodiments, the kinase inhibited by the compounds and compositions described herein is a phosphatidylinositol 3-kinase (PI3K). In some embodiments, the kinase inhibited by the compounds and compositions described herein is one or more of a PI3Kα, PI3Kδ, and PI3Kγ. In some embodiments, the kinase inhibited by the compounds and compositions described herein is a PI3Kα. In some embodiments, the kinase inhibited by the compounds and compositions described herein is a PI3Kα containing at least one of the following mutations: H1047R, E542K, and E545K.

Compounds or compositions of the disclosure can be useful in applications that benefit from inhibition of PI3K enzymes. For example, PI3K inhibitors of the present disclosure are useful for the treatment of cellular proliferative diseases generally. Compounds or compositions of the disclosure can be useful in applications that benefit from inhibition of PI3Kα enzymes. For example, PI3Kα inhibitors of the present disclosure are useful for the treatment of cellular proliferative diseases generally.

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

The activity of a compound utilized in this disclosure as an inhibitor of a PI3K kinase, for example, a PI3Kα, or a mutant thereof, may be assayed in vitro, in vivo or in a cell line. In vitro assays include assays that determine inhibition of either the phosphorylation activity and/or the subsequent functional consequences, or ATPase activity of an activated PI3Kα, or a mutant thereof. Alternative in vitro assays quantitate the ability of the inhibitor to bind to a a PI3Kα. Inhibitor binding may be measured by radiolabeling the inhibitor prior to binding, isolating the inhibitor/PI3Kα complex and determining the amount of radiolabel bound. Alternatively, inhibitor binding may be determined by running a competition experiment where new inhibitors are incubated with a PI3Kα bound to known radioligands. Representative in vitro and in vivo assays useful in assaying a PI3Kα inhibitor include those described and disclosed in the patent and scientific publications described herein. Detailed conditions for assaying a compound utilized in this disclosure as an inhibitor of a PI3Kα, or a mutant thereof, are set forth in the Examples below.

### Treatment of Disorders

Provided compounds are inhibitors of PI3Kα and are therefore useful for treating one or more disorders associated with activity of PI3Kα or mutants thereof. Thus, in certain embodiments, the present disclosure provides a method of treating a PI3Kα-mediated disorder in a subject, comprising administering a therapeutically effective amount of a compound of the present disclosure, or a pharmaceutically acceptable salt thereof, or a pharmaceutically acceptable composition of either of the foregoing, to a subject in need thereof. In certain embodiments, the present disclosure provides a method of treating a PI3Kα-mediated disorder in a subject comprising administering a therapeutically effective amount of a compound of the present disclosure, or a pharmaceutically acceptable composition thereof, to a subject in need thereof. In some embodiments, the subject has a mutant PI3Kα. In some embodiments, the subject has PI3Kα containing at least one of the following mutations: H1047R, E542K, and E545K.

As used herein, the term “PI3Kα-mediated” disorders, diseases, and/or conditions means any disease or other deleterious condition in which PI3Kα or a mutant thereof is known to play a role. Accordingly, another embodiment of the present disclosure relates to treating or lessening the severity of one or more diseases in which PI3Kα, or a mutant thereof, is known to play a role. Such PI3Kα-mediated disorders include, but are not limited to, cellular proliferative disorders (e.g. cancer). In some embodiments, the PI3Kα-mediated disorder is a disorder mediated by a mutant PI3Kα. In some embodiments, the PI3Kα-mediated disorder is a disorder mediated by a PI3Kα containing at least one of the following mutations: H1047R, E542K, and E545K.

In some embodiments, the present disclosure provides a method for treating a cellular proliferative disease, said method comprising administering to a patient in need thereof a therapeutically effective amount of a compound of the present disclosure, or a pharmaceutically acceptable salt thereof, or a pharmaceutically acceptable composition of either of the foregoing. In some embodiments, the present disclosure provides a method for treating a cellular proliferative disease, said method comprising administering to a patient in need thereof, a therapeutically effective amount of a compound of the present disclosure, or a pharmaceutically acceptable composition thereof.

In some embodiments, the method of treatment comprises the steps of: i) identifying a subject in need of such treatment; (ii) providing a disclosed compound, or a pharmaceutically acceptable salt thereof, and (iii) administering said provided compound in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K.

In some embodiments, the method of treatment comprises the steps of: i) identifying a subject in need of such treatment; (ii) providing a composition comprising a disclosed compound, or a pharmaceutically acceptable salt thereof, and (iii) administering said composition in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Cellular Proliferative Diseases

In some embodiments, the disorder is a cellular proliferative disease. In some embodiments, the cellular proliferative disease is cancer. In some embodiments, the cancer is a tumor. In some embodiments, the cancer is a solid tumor. In some embodiments, the cellular proliferative disease is a tumor and/or cancerous cell growth. In some embodiments, the cellular proliferative disease is a tumor. In some embodiments, the cellular proliferative disease is a solid tumor. In some embodiments, the cellular proliferative disease is a cancerous cell growth.

In some embodiments, the cancer is selected from sarcoma; lung; bronchus; prostate; breast (including sporadic breast cancers and sufferers of Cowden disease); pancreas; gastrointestinal; colon; rectum; carcinoma; colon carcinoma; adenoma; colorectal adenoma; thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary (including clear cell ovarian cancer); multiple myeloma; esophagus; a leukemia; acute myelogenous leukemia; chronic myelogenous leukemia; lymphocytic leukemia; myeloid leukemia; brain; a carcinoma of the brain; oral cavity and pharynx; larynx; small intestine; non-Hodgkin lymphoma; villous colon adenoma; a neoplasia; a neoplasia of epithelial character; lymphoma; a mammary carcinoma; basal cell carcinoma; squamous cell carcinoma; actinic keratosis; neck; head; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; a leukemia; acute myelogenous leukemia; chronic myelogenous leukemia; lymphocytic leukemia; myeloid leukemia; brain; oral cavity and pharynx; larynx; small intestine; neck; and head. In some embodiments, the cancer is selected from sarcoma; carcinoma; colon carcinoma; adenoma; colorectal adenoma; glioma; glioblastoma; melanoma; multiple myeloma; a carcinoma of the brain; non-Hodgkin lymphoma; villous colon adenoma; a neoplasia; a neoplasia of epithelial character; lymphoma; a mammary carcinoma; basal cell carcinoma; squamous cell carcinoma; actinic keratosis; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; brain; oral cavity and pharynx; larynx; small intestine; neck; and head. In some embodiments, the cancer is a leukemia. In some embodiments, the cancer is acute myelogenous leukemia; chronic myelogenous leukemia; lymphocytic leukemia; or myeloid leukemia.

In some embodiments, the cancer is breast cancer (including sporadic breast cancers and Cowden disease). In some embodiments, the cancer is breast cancer. In some embodiments, the cancer is ER+/HER2− breast cancer. In some embodiments, the cancer is ER+/HER2− breast cancer, and the subject is intolerant to, or ineligible for, treatment with alpelisib. In some embodiments, the cancer is sporadic breast cancer. In some embodiments, the cancer is Cowden disease.

In some embodiments, the cancer is ovarian cancer. In some embodiments, the ovarian cancer is clear cell ovarian cancer.

In some embodiments, the cellular proliferative disease has mutant PI3Kα. In some embodiments, the cancer has mutant PI3Kα. In some embodiments, the breast cancer has mutant PI3Kα. In some embodiments, the ovarian cancer has mutant PI3Kα.

In some embodiments, the cellular proliferative disease has PI3Kα containing at least one of the following mutations: H1047R, E542K, and E545K. In some embodiments, the cancer has PI3Kα containing at least one of the following mutations: H1047R, E542K, and E545K. In some embodiments, the breast cancer has PI3Kα containing at least one of the following mutations: H1047R, E542K, and E545K. In some embodiments, the ovarian cancer has PI3Kα containing at least one of the following mutations: H1047R, E542K, and E545K.

In some embodiments, the cancer is adenoma; carcinoma; sarcoma; glioma; glioblastoma; melanoma; multiple myeloma; or lymphoma. In some embodiments, the cancer is a colorectal adenoma or avillous colon adenoma. In some embodiments, the cancer is colon carcinoma; a carcinoma of the brain; a mammary carcinoma; basal cell carcinoma; or a squamous cell carcinoma. In some embodiments, the cancer is a neoplasia or a neoplasia of epithelial character. In some embodiments, the cancer is non-Hodgkin lymphoma. In some embodiments, the cancer is actinic keratosis; polycythemia vera; essential thrombocythemia; myelofibrosis with myeloid metaplasia; or Waldenstrom macroglobulinemia.

In some embodiments, the cellular proliferative disease displays overexpression or amplification of PI3Kα, somatic mutation of PIK3CA, germline mutations or somatic mutation of PTEN, or mutations and translocation of p85a that serve to up-regulate the p85-p110 complex. In some embodiments, the cellular proliferative disease displays overexpression or amplification of PI3Kα. In some embodiments, the cellular proliferative disease displays somatic mutation of PIK3CA. In some embodiments, the cellular proliferative disease displays germline mutations or somatic mutation of PTEN. In some embodiments, the cellular proliferative disease displays mutations and translocation of p85α that serve to up-regulate the p85-p110 complex.

### Additional Disorders

In some embodiments, the PI3Kα-mediated disorder is selected from the group consisting of: polycythemia vera, essential thrombocythemia, myelofibrosis with myeloid metaplasia, asthma, COPD, ARDS, PROS (PI3K-related overgrowth syndrome), venous malformation, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g. haemolytic anaemia, aplastic anaemia, pure red cell anaemia and idiopathic thrombocytopenia), systemic lupus erythematosus, polychondritis, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In some embodiments, the PI3Kα-mediated disorder is polycythemia vera, essential thrombocythemia, or myelofibrosis with myeloid metaplasia. In some embodiments, the PI3Kα-mediated disorder is asthma, COPD, ARDS, PROS (PI3K-related overgrowth syndrome), venous malformation, Loffler's syndrome, eosinophilic pneumonia, parasitic (in particular metazoan) infestation (including tropical eosinophilia), or bronchopulmonary aspergillosis. In some embodiments, the PI3Kα-mediated disorder is polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, or scleroderma. In some embodiments, the PI3Kα-mediated disorder is vitiligo, hypersensitivity angiitis, urticaria, bullous pemphigoid, lupus erythematosus, pemphisus, epidermolysis bullosa acquisita, or autoimmune haematogical disorders (e.g. haemolytic anaemia, aplastic anaemia, pure red cell anaemia and idiopathic thrombocytopenia). In some embodiments, the PI3Kα-mediated disorder is systemic lupus erythematosus, polychondritis, scleroderma, Wegener granulomatosis, dermatomyositis, chronic active hepatitis, myasthenia gravis, Steven-Johnson syndrome, idiopathic sprue, or autoimmune inflammatory bowel disease (e.g. ulcerative colitis and Crohn's disease).

In some embodiments, the PI3Kα-mediated disorder is endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, or psoriatic arthritis. In some embodiments, the PI3Kα-mediated disorder is glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, or reperfusion injuries. In some embodiments, the PI3Kα-mediated disorder is retinopathy, such as diabetic retinopathy or hyperbaric oxygen-induced retinopathy, and conditions characterized by elevated intraocular pressure or secretion of ocular aqueous humor, such as glaucoma.

### Routes of Administration and Dosage Forms

The compounds and compositions, according to the methods of the present disclosure, may be administered using any amount and any route of administration effective for treating or lessening the severity of the disorder (e.g. a proliferative disorder). The e^{YA}ct amount required will vary from subject to subject, depending on the species, age, and general condition of the subject, the severity of the infection, the particular agent, its mode of administration, and the like. Compounds of the disclosure are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Inhibition of Protein Kinases

According to one embodiment, the disclosure relates to a method of inhibiting protein kinase activity in a biological sample comprising the step of contacting said biological sample with a compound of this disclosure, or a composition comprising said compound. According to another embodiment, the disclosure relates to a method of inhibiting activity of a PI3K, or a mutant thereof, in a biological sample comprising the step of contacting said biological sample with a compound of this disclosure, or a composition comprising said compound. According to another embodiment, the disclosure relates to a method of inhibiting activity of PI3Kα, or a mutant thereof, in a biological sample comprising the step of contacting said biological sample with a compound of this disclosure, or a composition comprising said compound. In some embodiments, the PI3Kα is a mutant PI3Kα. In some embodiments, the PI3Kα contains at least one of the following mutations: H1047R, E542K, and E545K.

In another embodiment, the disclosure provides a method of selectively inhibiting PI3Kα over one or both of PI3Kδ and PI3Kγ. In some embodiments, a compound of the present disclosure is more than 5-fold selective over PI3Kδ and PI3Kγ. In some embodiments, a compound of the present disclosure is more than 10-fold selective over PI3Kδ and PI3Kγ. In some embodiments, a compound of the present disclosure is more than 50-fold selective over PI3Kδ and PI3Kγ. In some embodiments, a compound of the present disclosure is more than 100-fold selective over PI3Kδ and PI3Kγ. In some embodiments, a compound of the present disclosure is more than 200-fold selective over PI3Kδ and PI3Kγ. In some embodiments, the PI3Kα is a mutant PI3Kα. In some embodiments, the PI3Kα contains at least one of the following mutations: H1047R, E542K, and E545K.

In another embodiment, the disclosure provides a method of selectively inhibiting a mutant PI3Kα over a wild-type PI3Kα. In some embodiments, a compound of the present disclosure is more than 5-fold selective for mutant PI3Kα over wild-type PI3Kα. In some embodiments, a compound of the present disclosure is more than 10-fold selective for mutant PI3Kα over wild-type PI3Kα. In some embodiments, a compound of the present disclosure is more than 50-fold selective for mutant PI3Kα over wild-type PI3Kα. In some embodiments, a compound of the present disclosure is more than 100-fold selective for mutant PI3Kα over wild-type PI3Kα. In some embodiments, a compound of the present disclosure is more than 200-fold selective for mutant PI3Kα over wild-type PI3Kα. In some embodiments, the mutant PI3Kα contains at least one of the following mutations: H1047R, E542K, and E545K.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of a PI3K (for example, PI3Kα, or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting protein kinase activity in a patient comprising the step of administering to said patient a compound of the present disclosure, or a composition comprising said compound.

According to another embodiment, the disclosure relates to a method of inhibiting activity of a PI3K, or a mutant thereof, in a patient comprising the step of administering to said patient a compound of the present disclosure, or a composition comprising said compound. In some embodiments, the disclosure relates to a method of inhibiting activity of PI3Kα, or a mutant thereof, in a patient comprising the step of administering to said patient a compound of the present disclosure, or a composition comprising said compound. In some embodiments, the PI3Kα is a mutant PI3Kα. In some embodiments, the PI3Kα contains at least one of the following mutations: H1047R, E542K, and E545K.

According to another embodiment, the present disclosure provides a method for treating a disorder mediated by a PI3K, or a mutant thereof, in a patient in need thereof, comprising the step of administering to said patient a compound according to the present disclosure or pharmaceutically acceptable composition thereof. In some embodiments, the present disclosure provides a method for treating a disorder mediated by PI3Kα, or a mutant thereof, in a patient in need thereof, comprising the step of administering to said patient a compound according to the present disclosure or pharmaceutically acceptable composition thereof. In some embodiments, the PI3Kα is a mutant PI3Kα. In some embodiments, the PI3Kα contains at least one of the following mutations: H1047R, E542K, and E545K.

According to another embodiment, the present disclosure provides a method of inhibiting signaling activity of PI3Kα, or a mutant thereof, in a subject, comprising administering a therapeutically effective amount of a compound according to the present disclosure, or a pharmaceutically acceptable composition thereof, to a subject in need thereof. In some embodiments, the present disclosure provides a method of inhibiting PI3Kα signaling activity in a subject, comprising administering a therapeutically effective amount of a compound according to the present disclosure, or a pharmaceutically acceptable composition thereof, to a subject in need thereof. In some embodiments, the PI3Kα is a mutant PI3Kα. In some embodiments, the PI3Kα contains at least one of the following mutations: H1047R, E542K, and E545K. In some embodiments, the subject has a mutant PI3Kα. In some embodiments, the subject has PI3Kα containing at least one of the following mutations: H1047R, E542K, and E545K.

The compounds described herein can also inhibit PI3Kα function through incorporation into agents that catalyze the destruction of PI3Kα. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of PI3Kα to the E3 ligase will thus result in the destruction of the PI3Kα protein. The variable chain of atoms can include, for example, rings, heteroatoms, and/or repeating polymeric units. It can be rigid or flexible. It can be attached to the two portions described above using standard techniques in the art of organic synthesis.

### Combination Therapies

Depending upon the particular disorder, condition, or disease, to be treated, additional therapeutic agents, that are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

Additionally, PI3K serves as a second messenger node that integrates parallel signaling pathways, and evidence is emerging that the combination of a PI3K inhibitor with inhibitors of other pathways will be useful in treating cancer and cellular proliferative diseases.

Accordingly, in certain embodiments, the method of treatment comprises administering the compound or composition of the disclosure in combination with one or more additional therapeutic agents. In certain other embodiments, the methods of treatment comprise administering the compound or composition of the disclosure as the only therapeutic agent.

Approximately 20-30% of human breast cancers overexpress Her-2/neu-ErbB2, the target for the drug trastuzumab. Although trastuzumab has demonstrated durable responses in some patients expressing Her2/neu-ErbB2, only a subset of these patients respond. Recent work has indicated that this limited response rate can be substantially improved by the combination of trastuzumab with inhibitors of PI3K or the PI13K/AKT pathway (Chan et al., Breast Can. Res. Treat. 91:187 (2005), Woods Ignatoski et al., Brit. J. Cancer 82:666 (2000), Nagata et al., Cancer Cell 6:117 (2004)). Accordingly, in certain embodiments, the method of treatment comprises administering the compound or composition of the disclosure in combination with trastuzumab. In certain embodiments, the cancer is a human breast cancer that overexpresses Her-2/neu-ErbB2.

A variety of human malignancies express activating mutations or increased levels of Her1/EGFR and a number of antibody and small molecule inhibitors have been developed against this receptor tyrosine kinase including tarceva, gefitinib and erbitux. However, while EGFR inhibitors demonstrate anti-tumor activity in certain human tumors (e.g., NSCLC), they fail to increase overall patient survival in all patients with EGFR-expressing tumors. This may be rationalized by the fact that many downstream targets of Her1/EGFR are mutated or deregulated at high frequencies in a variety of malignancies, including the PI3K/Akt pathway.

For example, gefitinib inhibits the growth of an adenocarcinoma cell line in in vitro assays. Nonetheless, sub-clones of these cell lines can be selected that are resistant to gefitinib that demonstrate increased activation of the PI3/Akt pathway. Down-regulation or inhibition of this pathway renders the resistant sub-clones sensitive to gefitinib (Kokubo et al., Brit. J. Cancer 92:1711 (2005)). Furthermore, in an in vitro model of breast cancer with a cell line that harbors a PTEN mutation and over-expresses EGFR inhibition of both the PI3K/Akt pathway and EGFR produced a synergistic effect (She et al., Cancer Cell 8:287-297 (2005)). These results indicate that the combination of gefitinib and PI3K/Akt pathway inhibitors would be an attractive therapeutic strategy in cancer.

Accordingly, in certain embodiments, the method of treatment comprises administering the compound or composition of the disclosure in combination with an inhibitor of Her1/EGFR. In certain embodiments, the method of treatment comprises administering the compound or composition of the disclosure in combination with one or more of tarceva, gefitinib, and erbitux. In certain embodiments, the method of treatment comprises administering the compound or composition of the disclosure in combination with gefitinib. In certain embodiments, the cancer expresses activating mutations or increased levels of Her1/EGFR.

The combination of AEE778 (an inhibitor of Her-2/neu/ErbB2, VEGFR and EGFR) and RAD001 (an inhibitor of mTOR, a downstream target of Akt) produced greater combined efficacy that either agent alone in a glioblastoma xenograft model (Goudar et al., Mol. Cancer. Ther. 4:101-112 (2005)).

Anti-estrogens, such as tamoxifen, inhibit breast cancer growth through induction of cell cycle arrest that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to anti-estrogen resistance (Donovan, et al, J. Biol. Chem. 276:40888, (2001)). As reported by Donovan et al., inhibition of MAPK signaling through treatment with MEK inhibitor reversed the aberrant phosphorylation status of p27 in hormone refractory breast cancer cell lines and in so doing restored hormone sensitivity. Similarly, phosphorylation of p27Kip by Aid also abrogates its role to arrest the cell cycle (Viglietto et al., Nat. Med. 8:1145 (2002)).

Accordingly, in certain embodiments, the method of treatment comprises administering the compound or composition of the disclosure in combination with a treatment for a hormone-dependent cancer. In certain embodiments, the method of treatment comprises administering the compound or composition of the disclosure in combination with tamoxifen. In certain embodiments, the cancer is a hormone dependent cancer, such as breast and prostate cancers. By this use, it is aimed to reverse hormone resistance commonly seen in these cancers with conventional anticancer agents.

In hematological cancers, such as chronic myelogenous leukemia (CML), chromosomal translocation is responsible for the constitutively activated BCR-Abl tyrosine kinase. The afflicted patients are responsive to imatinib, a small molecule tyrosine kinase inhibitor, as a result of inhibition of Abl kinase activity. However, many patients with advanced stage disease respond to imatinib initially, but then relapse later due to resistance-conferring mutations in the Abl kinase domain. In vitro studies have demonstrated that BCR-Ab1 employs the Ras-Raf kinase pathway to elicit its effects. In addition, inhibiting more than one kinase in the same pathway provides additional protection against resistance-conferring mutations.

Accordingly, in another aspect, the compounds and compositions of the disclosure are used in combination with at least one additional agent selected from the group of kinase inhibitors, such as imatinib, in the treatment of hematological cancers, such as chronic myelogenous leukemia (CML). By this use, it is aimed to reverse or prevent resistance to said at least one additional agent.

Because activation of the PI3K/Akt pathway drives cell survival, inhibition of the pathway in combination with therapies that drive apoptosis in cancer cells, including radiotherapy and chemotherapy, will result in improved responses (Ghobrial et al., CA Cancer J. Clin 55:178-194 (2005)). As an example, combination of PI3 kinase inhibitor with carboplatin demonstrated synergistic effects in both in vitro proliferation and apoptosis assays as well as in in vivo tumor efficacy in a xenograft model of ovarian cancer (Westfall and Skinner, Mol. Cancer Ther. 4:1764-1771 (2005)).

In some embodiments, the one or more additional therapeutic agents is selected from antibodies, antibody-drug conjugates, kinase inhibitors, immunomodulators, and histone deacetylase inhibitors. Synergistic combinations with PIK3CA inhibitors and other therapeutic agents are described in, for example, Castel et al., Mol. Cell Oncol. (2014)1(3) e963447.

In some embodiments, the one or more additional therapeutic agent is selected from the following agents, or a pharmaceutically acceptable salt thereof: BCR-ABL inhibitors (see e.g. Ultimo et al. Oncotarget (2017) 8 (14) 23213-23227): e.g. imatinib, inilotinib, nilotinib, dasatinib, bosutinib, ponatinib, bafetinib, danusertib, saracatinib, PF03814735; ALK inhibitors (see e.g. Yang et al. Tumour Biol. (2014) 35 (10) 9759-67): e.g. crizotinib, NVP-TAE684, ceritinib, alectinib, brigatinib, entrecinib, lorlatinib; BRAF inhibitors (see e.g. Silva et al. Mol. Cancer Res. (2014) 12, 447-463): e.g. vemurafenib, dabrafenib; FGFR inhibitors (see e.g. Packer et al. Mol. Cancer Ther. (2017) 16(4) 637-648): e.g. infigratinib, dovitinib, erdafitinib, TAS-120, pemigatinib, BLU-554, AZD4547; FLT3 inhibitors: e.g. sunitinib, midostaurin, tanutinib, sorafenib, lestaurtinib, quizartinib, and crenolanib; MEK Inhibitors (see e.g. Jokinen et al. Ther. Adv. Med. Oncol. (2015) 7(3) 170-180): e.g. trametinib, cobimetinib, binimetinib, selumetinib; ERK inhibitors: e.g. ulixertinib, MK 8353, LY 3214996; KRAS inhibitors: e.g. AMG-510, MRTX849, ARS-3248; Tyrosine kinase inhibitors (see e.g. Makhov et al. Mol. Cancer. Ther. (2012) 11(7) 1510-1517): e.g. erlotinib, linifanib, sunitinib, pazopanib; Epidermal growth factor receptor (EGFR) inhibitors (see e.g. She et al. BMC Cancer (2016) 16, 587): gefitnib, osimertinib, cetuximab, panitumumab; HER2 receptor inhibitors (see e.g. Lopez et al. Mol. Cancer Ther. (2015) 14(11) 2519-2526): e.g. trastuzumab, pertuzumab, neratinib, lapatinib, lapatinib; MET inhibitors (see e.g. Hervieu et al. Front. Mol. Biosci. (2018) 5, 86): e.g. crizotinib, cabozantinib; CD20 antibodies: e.g. rituximab, tositumomab, ofatumumab; DNA Synthesis inhibitors: e.g. capecitabine, gemcitabine, nelarabine, hydroxycarbamide; Antineoplastic agents (see e.g. Wang et al. Cell Death & Disease (2018) 9, 739): e.g. o^{YA}liplatin, carboplatin, cisplatin; Immunomodulators: e.g. afutuzumab, lenalidomide, thalidomide, pomalidomide; CD40 inhibitors: e.g. dacetuzumab; Pro-apoptotic receptor agonists (PARAs): e.g. dulanermin; Heat Shock Protein (HSP) inhibitors (see e.g. Chen et al. Oncotarget (2014) 5 (9). 2372-2389): e.g. tanespimycin; Hedgehog antagonists (see e.g. Chaturvedi et al. Oncotarget (2018) 9 (24), 16619-16633): e.g. vismodegib; Proteasome inhibitors (see e.g. Lin et al. Int. J. Oncol. (2014) 44 (2), 557-562): e.g. bortezomib; PI3K inhibitors: e.g. pictilisib, dactolisib, alpelisib, buparlisib, taselisib, idelalisib, duvelisib, umbralisib; SHP2 inhibitors (see e.g. Sun et al. Am. J. Cancer Res. (2019) 9 (1), 149-159: e.g. SHP099, RMC-4550, RMC-4630); BCL-2 inhibitors (see e.g. Bojarczuk et al. Blood (2018) 133 (1), 70-80): e.g. venetoclax; Aromatase inhibitors (see e.g. Mayer et al. Clin. Cancer Res. (2019) 25 (10), 2975-2987): exemestane, letrozole, anastrozole, fulvestrant, tamoxifen; mTOR inhibitors (see e.g. Woo et al. Oncogenesis (2017) 6, e385): e.g. temsirolimus, ridaforolimus, everolimus, sirolimus; CTLA-4 inhibitors (see e.g. O'Donnell et al. (2018) 48, 91-103): e.g. tremelimumab, ipilimumab; PD1 inhibitors (see O'Donnell, supra): e.g. nivolumab, pembrolizumab; an immunoadhesin; Other immune checkpoint inhibitors (see e.g. Zappasodi et al. Cancer Cell (2018) 33, 581-598, where the term “immune checkpoint” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta): e.g. pidilizumab, AMP-224; PDL1 inhibitors (see e.g. O'Donnell supra): e.g. MSB0010718C; YW243.55.570, MPDL3280A; MEDI-4736, MSB-0010718C, or MDX-1105; Histone deacetylase inhibitors (HDI, see e.g. Rahmani et al. Clin. Cancer Res. (2014) 20(18), 4849-4860): e.g. vorinostat; Androgen Receptor inhibitors (see e.g. Thomas et al. Mol. Cancer Ther. (2013) 12(11), 2342-2355): e.g. enzalutamide, abiraterone acetate, orteronel, galeterone, seviteronel, bicalutamide, flutamide; Androgens: e.g. fluoxymesterone; CDK4/6 inhibitors (see e.g. Gul et al. Am. J. Cancer Res. (2018) 8(12), 2359-2376): e.g. alvocidib, palbociclib, ribociclib, trilaciclib, abemaciclib.

In some embodiments, the one or more additional therapeutic agent is selected from the following agents: anti-FGFR antibodies; FGFR inhibitors, cytotoxic agents; Estrogen Receptor-targeted or other endocrine therapies, immune-checkpoint inhibitors, CDK inhibitors, Receptor Tyrosine Kinase inhibitors, BRAF inhibitors, MEK inhibitors, other PI3K inhibitors, SHP2 inhibitors, and SRC inhibitors. (See Katoh, Nat. Rev. Clin. Oncol. (2019), 16:105-122; Chae, et al. Oncotarget (2017), 8:16052-16074; Formisano et al., Nat. Comm. (2019), 10:1373-1386; and references cited therein.)

The structure of the active compounds identified by code numbers, generic or trade names may be taken from the actual edition of the standard compendium “The Merck Index” or from databases, e.g. Patents International (e.g. IMS World Publications).

A compound of the current disclosure may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current disclosure can be administered alone or in combination with one or more other therapeutic compounds, possible combination therapy taking the form of fixed combinations or the administration of a compound of the disclosure and one or more other therapeutic compounds being staggered or given independently of one another, or the combined administration of fixed combinations and one or more other therapeutic compounds. A compound of the current disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a compound of the current disclosure, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.01-100 mg/kg body weight/day of an inventive compound can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this disclosure, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this disclosure are another embodiment of the present disclosure.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to other classes and subclasses and species of each of these compounds, as described herein. Additional compounds of the disclosure were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. The starting materials for the Examples are either commercially available or are readily prepared by standard methods from known materials.

### List of Abbreviations

aq: aqueous
Ac: acetyl
ACN or MeCN: acetonitrile
AmF: ammonium formate
anhyd.: anhydrous
BINAP: (±)-2,2′-Bis(diphenylphosphino)-1,1′-binaphthalene
Bn: Benzyl
conc.: concentrated
DBU: 1,8-Diazabicyclo[5.4.0]undec-7-ene
DCE: Dichloroethane
DCM: Dichloromethane
DIPEA: Diisopropylamine
DMF: N,N-dimethylformamide
DMP: Dess-Martin periodinane
DMPU: N,N′-Dimethylpropyleneurea
DMSO: dimethylsulfoxide
DIPEA: diisopropylethylamine
EA or EtOAc: ethyl acetate
EDCI, EDC, or EDAC: 1-Ethyl-3-(3-dimethylaminopropyl)carbodiimide
equiv or eq: molar equivalents
Et: ethyl
HATU: 1-[Bis(dimethylamino)methylene]-1H-1,2,3-triazolo[4,5-b]pyridinium 3-oxid He^{YA}fluorophosphate
HPLC: high pressure liquid chromatography
LCMS or LC-MS: liquid chromatography-mass spectrometry
Ms: methanesulfonyl
NBS: N-bromosuccinimide
NMR: nuclear magnetic resonance
PE: petroleum ether
PMB: p-methoxybenzyl
rt or RT: room temperature
sat: saturated
TBS: tert-butyldimethylsilyl
TEA: triethylamine
Tf: trifluoromethanesulfonate
TFA: trifluoroacetic acid
THF: tetrahydrofuran
TLC: thin layer chromatography
Tol: toluene
UV: ultra violet

### Example 1

### N-(2-((2-chloro-5-fluorophenyl)amino)-5-(1-(difluoromethyl)-1H-pyrazol-4-yl)pyridin-3-yl)-3-fluoro-5-(trifluoromethyl)benzamide (I-803)

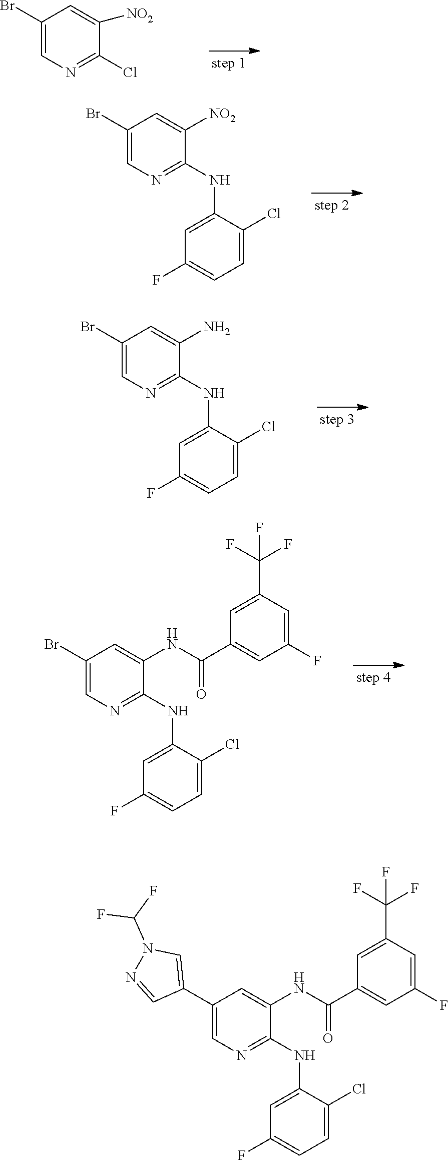

### Step 1. 5-bromo-N-(2-chloro-5-fluorophenyl)-3-nitropyridin-2-amine

A microwave vial was charged with 5-bromo-2-chloro-3-nitropyridine (1.5 g, 6.31 mmol), 2-chloro-5-fluoroaniline (1.37 g, 9.46 mmol), methanesulfonic acid (909 mg, 9.46 mmol) and a stirbar. Dioxane (10 mL) was added, the vial was sealed, and the mixture was stirred in the microwave at 180° C. for 1 h. The resulted solution was evaporated and purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in 5-bromo-N-(2-chloro-5-fluorophenyl)-3-nitropyridin-2-amine (700 mg, 2.01 mmol, 32.1%) as an orange red solid. m/z (ES+) [M+H]+=347.95; HPLC tR=1.842 min.

### Step 2. 5-bromo-N2-(2-chloro-5-fluorophenyl)pyridine-2,3-diamine

A round bottomed flask was charged with 5-bromo-N-(2-chloro-5-fluorophenyl)-3-nitropyridin-2-amine (700 mg, 2.01 mmol), iron (558 mg, 10.0 mmol), ammonium chloride (430 mg, 8.04 mmol) and a stirbar. Water (20 mL) and ethanol (30 mL) were added, and the solution was stirred at 90° C. for 2 h. Filtered the mixture, and extracted the filtrate with EA. The organic layer was dried over Na₂SO₄ and evaporated. This resulted in 5-bromo-N2-(2-chloro-5-fluorophenyl)pyridine-2,3-diamine (400 mg, 1.26 mmol, 62.8%) as a yellow solid. m/z (ES+) [M+H]+=318.00; HPLC tR=0.867 min.

### Step 3. N-(5-bromo-2-((2-chloro-5-fluorophenyl)amino)pyridin-3-yl)-3-fluoro-5-(trifluoromethyl)benzamide

A round bottomed flask was charged with 5-bromo-N2-(2-chloro-5-fluorophenyl)pyridine-2,3-diamine (200 mg, 631 μmol), 3-fluoro-5-(trifluoromethyl)benzoic acid (121 mg, 631 μmol), HBTU (358 mg, 946 μmol), 4-methylmorpholine (207 μL, 1.89 mmol) and a stirbar. Dimethylformamide (3 mL) was added, and the solution was stirred at 80° C. overnight. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in N-{5-bromo-2-[(2-chloro-5-fluorophenyl)amino]pyridin-3-yl}-3-fluoro-5-(trifluoromethyl)benzamide (110 mg, 217 μmol, 34.4%) as a light red solid. m/z (ES+) [M+H]+=508.15; HPLC tR=2.162 min.

### Step 4. N-(2-((2-chloro-5-fluorophenyl)amino)-5-(1-(difluoromethyl)-1H-pyrazol-4-yl)pyridin-3-yl)-3-fluoro-5-(trifluoromethyl)benzamide

A round bottomed flask was charged with N-{5-bromo-2-[(2-chloro-5-fluorophenyl)amino]pyridin-3-yl}-3-fluoro-5-(trifluoromethyl)benzamide (50 mg, 98.6 μmol), 1-(difluoromethyl)-4-(4,4,5,5-tetramethyl-1,3,2-dioxaborolan-2-yl)-1H-pyrazole (28.7 mg, 118 μmol), [1,1′-bis(diphenylphosphino)ferrocene]dichloropalladium(II) (14.4 mg, 19.7 μmol), potassium carbonate (40.7 mg, 295 μmol) and a stirbar. Dioxane (2.5 mL) and water (0.5 ml) were added, and the solution was stirred at 90° C. for 1 h under N₂. The resulting crude material was purified using Prep-HPLC with following conditions: Column: XBridge Prep OBD C18 Column, 30×150 mm 5 um; Mobile Phase A: Water (10 MMOL/L NH4HCO3+0.1% NH3·H2O), Mobile Phase B: ACN; Flow rate: 60 mL/min; Gradient: 45% B to 85% B in 8 min; 220 nm; RT1: 7.23 min. Lyophilization yielded N-{2-[(2-chloro-5-fluorophenyl)amino]-5-[1-(difluoromethyl)-1H-pyrazol-4-yl]pyridin-3-yl}-3-fluoro-5-(trifluoromethyl)benzamide (12.7 mg, 23.3 μmol, 23.6%) as a white solid. m/z (ES+) [M+H]+=544.15. ¹H NMR (DMSO-d6, 400 MHz) 10.77 (1H, s), 8.77 (1H, s), 8.66 (1H, d, J=2.3 Hz), 8.33 (1H, s), 8.29 (1H, s), 8.22-8.08 (3H, m), 8.04 (2H, t, J=8.5 Hz), 7.80 (1H, d, J=59.2 Hz), 7.51 (1H, dd, J=8.9, 6.0 Hz), 6.86 (1H, ddd, J=8.9, 7.9, 3.1 Hz).

Additional compounds prepared according to the methods of Example 1 are listed in Table 2 below. Corresponding ¹H NMR and mass spectrometry characterization for these compounds are described in Table 1. Certain compounds in Table 2 below were prepared with other compounds whose preparation is described further in the Examples herein.

TABLE 2
Additional Compounds

| Compound | Compound | Compound |
|---|---|---|
| I-7 | I-95 | I-119 |
| I-9 | I-96 | I-120 |
| I-45 | I-97 | I-121 |
| I-46 | I-98 | I-122 |
| I-56 | I-99 | I-123 |
| I-57 | I-100 | I-124 |
| I-61 | I-101 | I-125 |
| I-71 | I-102 | I-126 |
| I-74 | I-103 | I-127 |
| I-75 | I-104 | I-128 |
| I-76 | I-105 | I-129 |
| I-77 | I-106 | I-130 |
| I-78 | I-107 | I-131 |
| I-79 | I-108 | I-132 |
| I-80 | I-109 | I-145 |
| I-81 | I-110 | I-146 |
| I-82 | I-111 | I-147 |
| I-83 | I-112 | I-148 |
| I-84 | I-113 | I-767 |
| I-89 | I-114 | I-804 |
| I-90 | I-115 | I-811 |
| I-91 | I-116 | I-816 |
| I-92 | I-117 | I-817 |
| I-93 | I-118 | I-818 |
| I-94 | | |

### Example 2

### 6-((2-chloro-5-fluorophenyl)amino)-5-((5-chloroisoquinolin-1-yl)amino)-N-methylnicotinamide (I-805)

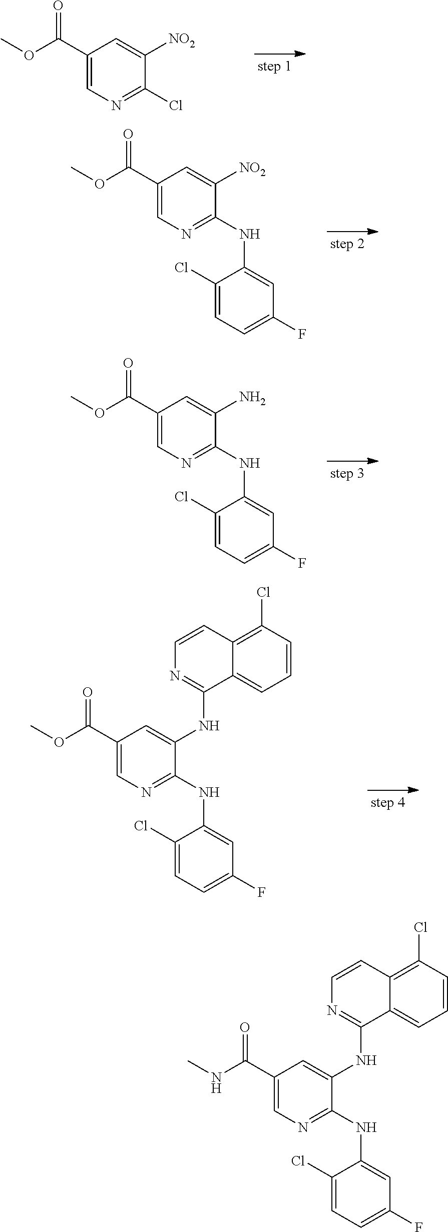

### Step 1. methyl 6-((2-chloro-5-fluorophenyl)amino)-5-nitronicotinate

A microwave vial was charged with methyl 6-chloro-5-nitropyridine-3-carboxylate (1.2 g, 5.54 mmol), 2-chloro-5-fluoroaniline (1.20 g, 8.30 mmol), methanesulfonic acid (797 mg, 8.30 mmol) and a stirbar. Dioxane (10 mL) was added, the vial was sealed, and the mixture was stirred in the microwave at 180° C. for 1 h. The resulted solution was evaporated and purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in methyl 6-[(2-chloro-5-fluorophenyl)amino]-5-nitropyridine-3-carboxylate (300 mg, 921 μmol, 16.6%) as a brick red solid. m/z (ES+) [M+H]+=326.10; HPLC tR=1.972 min.

### Step 2. methyl 5-amino-6-((2-chloro-5-fluorophenyl)amino)nicotinate

A round bottomed flask was charged with methyl 6-[(2-chloro-5-fluorophenyl)amino]-5-nitropyridine-3-carboxylate (600 mg, 1.84 mmol), iron (513 mg, 9.20 mmol), ammonium chloride (393 mg, 7.36 mmol) and a stirbar. Water (20 mL) and ethanol (30 ml) were added, and the solution was stirred at 80° C. for 2 h. Filtered the mixture and extracted the filtrate with EA. The organic layer was dried over Na₂SO₄ and evaporated. This resulted in methyl 5-amino-6-[(2-chloro-5-fluorophenyl)amino]pyridine-3-carboxylate (300 mg, 1.01 mmol, 55.1%) as a yellow solid. m/z (ES+) [M+H]+=296.10; HPLC tR=1.478 min.

### Step 3. methyl 6-((2-chloro-5-fluorophenyl)amino)-5-((5-chloroisoquinolin-1-yl)amino)nicotinate

A round bottomed flask was charged with methyl 5-amino-6-[(2-chloro-5-fluorophenyl)amino]pyridine-3-carboxylate (100 mg, 338 μmol), 1,5-dichloroisoquinoline (200 mg, 1.01 mmol), and a stirbar, and the solution was stirred at 190° C. for 1 h. Then purified the crude product by Prep-TLC (PE:EA=2:1). This resulted in methyl 6-[(2-chloro-5-fluorophenyl)amino]-5-[(5-chloroisoquinolin-1-yl)amino]pyridine-3-carboxylate (80 mg, 174 μmol, 51.9%) as a purple solid. m/z (ES+) [M+H]+=457.20; HPLC tR=1.727 min.

### Step 4. 6-((2-chloro-5-fluorophenyl)amino)-5-((5-chloroisoquinolin-1-yl)amino)-N-methylnicotinamide

A round bottomed flask was charged with methyl 6-[(2-chloro-5-fluorophenyl)amino]-5-[(5-chloroisoquinolin-1-yl)amino]pyridine-3-carboxylate (70 mg, 153 μmol), methanamine (19.6 g, 631 mmol) in water, and a stirbar. Methanol (10 mL) was added, and the solution was stirred at rt for 24 h. The resulting crude material was purified using Prep-HPLC with following conditions: Column: XBridge Shield RP18 OBD Column, 30*150 mm, 5 um; Mobile Phase A: Water (10 MMOL/L NH4HCO3+0.1% NH3·H2O), Mobile Phase B: ACN; Flow rate: 60 mL/min; Gradient: 58% B to 71% B in 8 min; 254/220 nm; RT1: 7.37 min. Lyophilization yielded 6-[(2-chloro-5-fluorophenyl)amino]-5-[(5-chloroisoquinolin-1-yl)amino]-N-methylpyridine-3-carboxamide (7 mg, 15.3 μmol, 10.0%) as a yellow solid. m/z (ES+) [M+H]+=456.05. ¹H NMR (DMSO-d6, 400 MHz) 9.57 (1H, s), 8.70 (1H, d, J=2.2 Hz), 8.51 (2H, dd, J=17.6, 6.7 Hz), 8.30 (1H, dd, J=11.6, 3.0 Hz), 8.23-8.12 (2H, m), 8.05 (1H, d, J=6.1 Hz), 7.97 (1H, d, J=7.5 Hz), 7.68 (1H, t, J=8.0 Hz), 7.57-7.34 (2H, m), 6.83 (1H, td, J=8.4, 3.0 Hz), 2.81 (3H, d, J=4.5 Hz).

Additional compounds prepared according to the methods of Example 2 are listed in Table 3 below. Corresponding ¹H NMR and mass spectrometry characterization for these compounds are described in Table 1. Certain compounds in Table 3 below were prepared with other compounds whose preparation is described further in the Examples herein.

TABLE 3
Additional Compounds
I-266
I-327

### Example 3

### N-(2-chloro-5-fluorophenyl)-3-[7-fluoro-5-(trifluoromethyl)-1H-1,3-benzodiazol-2-yl]pyridin-2-amine (I-829)

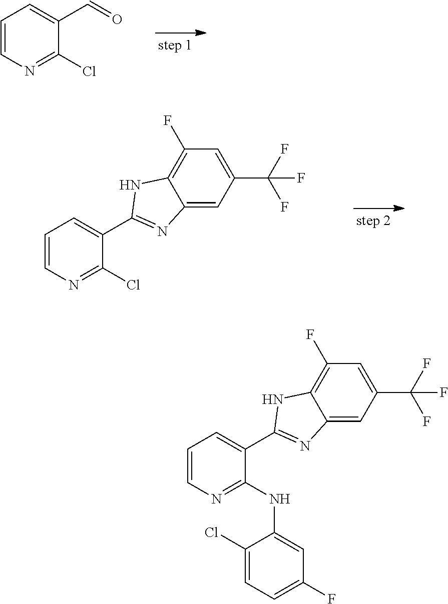 [SMILES: O=Cc1cccnc1Cl ; Fc1cc(C(F)(F)F)cc2nc(-c3cccnc3Cl)[nH]c12 ; Fc1ccc(Cl)c(Nc2ncccc2-c2nc3cc(C(F)(F)F)cc(F)c3[nH]2)c1]

### Step 1. 2-(2-chloropyridin-3-yl)-7-fluoro-5-(trifluoromethyl)-1H-1,3-benzodiazole

To a stirred solution of 2-chloropyridine-3-carbaldehyde (200 mg, 1.41 mmol), 3-fluoro-5-(trifluoromethyl)benzene-1,2-diamine (273 mg, 1 eq) in DMF (2 mL) was added KHSO₅ (139 mg, 0.65 eq) in H₂O (0.2 mL) dropwise at 0° C. and the resulting solution was stirred at room temperature overnight under nitrogen atmosphere. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in 2-(2-chloropyridin-3-yl)-7-fluoro-5-(trifluoromethyl)-1H-1,3-benzodiazole (200 mg, 633 μmol, 44.9%) as a yellow solid. Lcms: Rt=0.928 min, m/z=315.90 (M+1)+.

### Step 2. N-(2-chloro-5-fluorophenyl)-3-[7-fluoro-5-(trifluoromethyl)-1H-1,3-benzodiazol-2-yl]pyridin-2-amine

A microwave vial was charged with 2-(2-chloropyridin-3-yl)-7-fluoro-5-(trifluoromethyl)-1H-1,3-benzodiazole (150 mg, 475 μmol), 2-chloro-5-fluoroaniline (138 mg, 2 eq), MsOH (91.2 mg, 2.0 eq) and a stirbar. Dioxane (2.0 mL) was added, the vial was sealed, and the mixture was stirred in the microwave at 180° C. for 1 h. The resulting mixture was concentrated under reduced pressure and was dissolved in DMF. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in N-(2-chloro-5-fluorophenyl)-3-[7-fluoro-5-(trifluoromethyl)-1H-1,3-benzodiazol-2-yl]pyridin-2-amine (25.7 mg, 60.5 μmol, 12.7%) as an amorphous solid. Lcms: Rt=2.263 min, m/z=425.05 (M+1)+. ¹H NMR (400 MHz, DMSO-d6): 6.88 (1H, ddd), 7.20 (1H, dd), 7.45-7.67 (2H, m), 7.85 (1H, d), 8.44-8.70 (2H, m), 8.86 (1H, ddd), 12.35 (1H, d), 14.01 (1H, d).

### Example 4

### N-{2-[(2-chloro-5-fluorophenyl)amino]-5-(methylcarbamoyl)pyridin-3-yl}-2,3-dihydro-1H-indole-1-carboxamide (I-797)

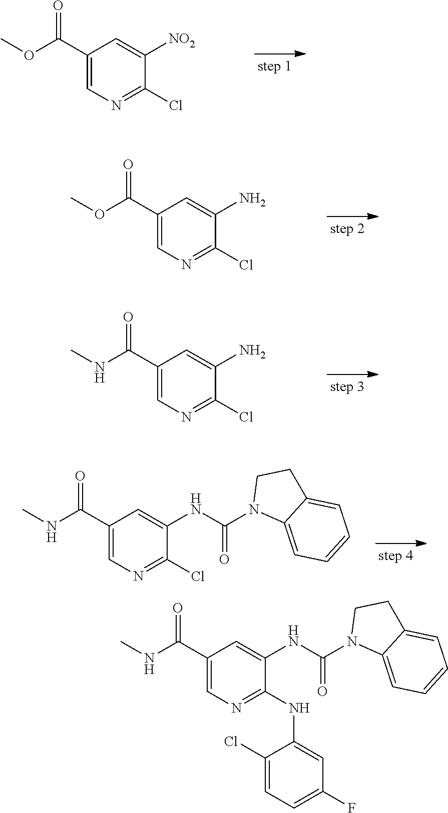

### Step 1. methyl 5-amino-6-chloropyridine-3-carboxylate

To a solution of methyl 6-chloro-5-nitropyridine-3-carboxylate (8 g, 36.9 mmol) in MeOH (80.0 mL) was added Fe (10.6 g, 5.2 eq) and NH₄Cl (10.3 g, 5.3 eq). The reaction mixture was stirred at 75° C. for 2 h. The resulting mixture was filtered, the filter cake was washed with MeOH. The filtrate was concentrated under reduced pressure. This resulted in methyl 5-amino-6-chloropyridine-3-carboxylate (3.8 g, 20.3 mmol, 55.2%) as a white solid. Lcms: Rt=1.068 min, m/z=187.05 (M+1)+.

### Step 2. 5-amino-6-chloro-N-methylpyridine-3-carboxamide

To a stirred solution of methyl 5-amino-6-chloropyridine-3-carboxylate (3 g, 16.0 mmol) in MeOH (30 mL) was added methanamine (40% wt, in H₂O, 14.9 g, 12 eq) and the resulting solution was stirred at room temperature for 4 h. The reaction was quenched with water and extracted with EA. The organic layer was washed with brine, dried over Na₂SO₄ and evaporated. This resulted in 5-amino-6-chloro-N-methylpyridine-3-carboxamide (2 g, 10.7 mmol, 67.5%) as alight yellow solid. Lcms: Rt=0.773 min, m/z=186.10 (M+1)+.

### Step 3. N-[2-chloro-5-(methylcarbamoyl)pyridin-3-yl]-2,3-dihydro-1H-indole-1-carboxamide

To a solution of 5-amino-6-chloro-N-methylpyridine-3-carboxamide (400 mg, 2.15 mmol) and TEA (477 mg, 2.2 eq) in DCM (1 mL) was added triphosgene (255 mg, 0.4 eq). After stirring at 0° C. for 2 h, 2,3-dihydro-1H-indole (306 mg, 1.2 eq) was added, and the resulting mixture was stirred at 0° C. for 1 h. The reaction was quenched with water and extracted with EA. The organic layer was washed with brine, dried over Na₂SO₄ and evaporated. This resulted in N-[2-chloro-5-(methylcarbamoyl)pyridin-3-yl]-2,3-dihydro-1H-indole-1-carboxamide (200 mg, 604 μmol, 28.1%) as alight yellow solid. Lcms: Rt=1.197 min, m/z=331.15 (M+1)+.

### Step 4. N-{2-[(2-chloro-5-fluorophenyl)amino]-5-(methylcarbamoyl)pyridin-3-yl}-2,3-dihydro-1H-indole-1-carboxamide

A microwave vial was charged with N-[2-chloro-5-(methylcarbamoyl)pyridin-3-yl]-2,3-dihydro-1H-indole-1-carboxamide (400 mg, 1.20 mmol), 2-chloro-5-fluoroaniline (349 mg, 2 eq), Cs₂CO₃ (781 mg, 2 eq), Pd₂(dba)₃ (109 mg, 0.1 eq), Xantphos (104 mg, 0.15 eq) and a stirbar. Dioxane (8 mL) was added, the vial was sealed, and the mixture was stirred in the microwave at 130° C. for 30 min. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in N-{2-[(2-chloro-5-fluorophenyl)amino]-5-(methylcarbamoyl)pyridin-3-yl}-2,3-dihydro-1H-indole-1-carboxamide (16.0 mg, 36.3 μmol, 3.03%) as a white amorphous solid. Lcms: Rt=0.976 min, m/z=440.10 (M+1)+. ¹H NMR (400 MHz, DMSO-d6): 2.81 (3H, d), 3.24 (2H, t), 4.20 (2H, t), 6.88 (1H, ddd), 6.94 (1H, td), 7.10-7.19 (1H, m), 7.24 (1H, d), 7.52 (1H, dd), 7.89 (1H, d), 8.10 (1H, d), 8.17-8.28 (1H, m), 8.40-8.50 (2H, m), 8.65 (1H, d), 8.74 (1H, s).

Additional compounds prepared according to the methods of Example 4 are listed in Table 4 below. Corresponding ¹H NMR and mass spectrometry characterization for these compounds are described in Table 1. Certain compounds in Table 4 below were prepared with other compounds whose preparation is described further in the Examples herein.

TABLE 4
Additional Compounds

| Compound | Compound | Compound |
|---|---|---|
| I-1 | I-22 | I-50 |
| I-2 | I-27 | I-51 |
| I-3 | I-28 | I-52 |
| I-4 | I-29 | I-53 |
| I-5 | I-30 | I-54 |
| I-6 | I-33 | I-64 |
| I-8 | I-34 | I-65 |
| I-10 | I-35 | I-66 |
| I-11 | I-36 | I-67 |
| I-12 | I-38 | I-85 |
| I-13 | I-39 | I-86 |
| I-14 | I-40 | I-87 |
| I-15 | I-41 | I-133 |
| I-17 | I-43 | I-134 |
| I-18 | I-44 | I-135 |
| I-19 | I-47 | I-136 |
| I-20 | I-48 | I-137 |
| I-138 | I-180 | I-225 |
| I-139 | I-182 | I-226 |
| I-140 | I-183 | I-230 |
| I-141 | I-184 | I-231 |
| I-142 | I-185 | I-232 |
| I-143 | I-187 | I-233 |
| I-149 | I-188 | I-234 |
| I-150 | I-189 | I-235 |
| I-154 | I-190 | I-236 |
| I-155 | I-191 | I-237 |
| I-157 | I-192 | I-238 |
| I-158 | I-193 | I-239 |
| I-159 | I-195 | I-240 |
| I-160 | I-196 | I-241 |
| I-161 | I-197 | I-242 |
| I-162 | I-198 | I-243 |
| I-163 | I-199 | I-246 |
| I-164 | I-200 | I-247 |
| I-165 | I-201 | I-248 |
| I-166 | I-202 | I-249 |
| I-167 | I-204 | I-250 |
| I-168 | I-205 | I-259 |
| I-169 | I-206 | I-260 |
| I-170 | I-207 | I-261 |
| I-171 | I-211 | I-262 |
| I-173 | I-212 | I-263 |
| I-174 | I-213 | I-264 |
| I-175 | I-219 | I-265 |
| I-176 | I-222 | I-267 |
| I-177 | I-223 | I-269 |
| I-178 | I-224 | I-274 |
| I-275 | I-319 | I-370 |
| I-276 | I-320 | I-371 |
| I-283 | I-322 | I-372 |
| I-284 | I-324 | I-373 |
| I-285 | I-325 | I-374 |
| I-286 | I-326 | I-375 |
| I-287 | I-328 | I-376 |
| I-288 | I-330 | I-377 |
| I-289 | I-331 | I-378 |
| I-290 | I-332 | I-379 |
| I-291 | I-333 | I-380 |
| I-292 | I-334 | I-381 |
| I-293 | I-335 | I-382 |
| I-294 | I-339 | I-383 |
| I-295 | I-340 | I-384 |
| I-296 | I-341 | I-385 |
| I-297 | I-342 | I-386 |
| I-298 | I-343 | I-387 |
| I-299 | I-346 | I-388 |
| I-300 | I-354 | I-389 |
| I-301 | I-355 | I-391 |
| I-302 | I-356 | I-392 |
| I-303 | I-357 | I-393 |
| I-304 | I-361 | I-394 |
| I-305 | I-362 | I-395 |
| I-306 | I-364 | I-396 |
| I-311 | I-365 | I-397 |
| I-312 | I-366 | I-399 |
| I-313 | I-367 | I-400 |
| I-316 | I-368 | I-401 |
| I-318 | I-369 | I-402 |
| I-403 | I-439 | I-470 |
| I-404 | I-440 | I-471 |
| I-405 | I-441 | I-472 |
| I-406 | I-442 | I-474 |
| I-407 | I-443 | I-475 |
| I-408 | I-444 | I-476 |
| I-409 | I-445 | I-477 |
| I-410 | I-446 | I-478 |
| I-414 | I-447 | I-479 |
| I-415 | I-448 | I-480 |
| I-416 | I-449 | I-481 |
| I-417 | I-450 | I-482 |
| I-418 | I-451 | I-483 |
| I-419 | I-452 | I-484 |
| I-420 | I-453 | I-485 |
| I-421 | I-454 | I-486 |
| I-422 | I-455 | I-487 |
| I-423 | I-456 | I-488 |
| I-424 | I-457 | I-489 |
| I-425 | I-458 | I-490 |
| I-426 | I-459 | I-491 |
| I-427 | I-460 | I-492 |
| I-428 | I-461 | I-493 |
| I-429 | I-462 | I-494 |
| I-430 | I-463 | I-495 |
| I-431 | I-464 | I-496 |
| I-432 | I-465 | I-497 |
| I-434 | I-466 | I-499 |
| I-435 | I-467 | I-500 |
| I-437 | I-468 | I-501 |
| I-438 | I-469 | I-502 |
| I-503 | I-534 | I-572 |
| I-504 | I-535 | I-573 |
| I-505 | I-536 | I-574 |
| I-506 | I-537 | I-575 |
| I-507 | I-538 | I-577 |
| I-508 | I-539 | I-578 |
| I-509 | I-540 | I-579 |
| I-510 | I-541 | I-580 |
| I-511 | I-542 | I-581 |
| I-512 | I-544 | I-582 |
| I-513 | I-545 | I-583 |
| I-514 | I-546 | I-584 |
| I-515 | I-547 | I-585 |
| I-516 | I-551 | I-586 |
| I-517 | I-555 | I-587 |
| I-518 | I-556 | I-588 |
| I-519 | I-557 | I-590 |
| I-520 | I-558 | I-591 |
| I-521 | I-559 | I-592 |
| I-522 | I-560 | I-593 |
| I-523 | I-561 | I-594 |
| I-524 | I-562 | I-595 |
| I-525 | I-563 | I-596 |
| I-526 | I-564 | I-598 |
| I-527 | I-565 | I-599 |
| I-528 | I-566 | I-601 |
| I-529 | I-567 | I-602 |
| I-530 | I-568 | I-603 |
| I-531 | I-569 | I-604 |
| I-532 | I-570 | I-605 |
| I-533 | I-571 | I-606 |
| I-608 | I-648 | I-695 |
| I-609 | I-649 | I-696 |
| I-610 | I-650 | I-697 |
| I-611 | I-651 | I-698 |
| I-612 | I-652 | I-699 |
| I-614 | I-653 | I-700 |
| I-615 | I-654 | I-701 |
| I-616 | I-655 | I-702 |
| I-617 | I-656 | I-703 |
| I-618 | I-659 | I-704 |
| I-619 | I-660 | I-705 |
| I-620 | I-661 | I-706 |
| I-621 | I-662 | I-707 |
| I-622 | I-663 | I-708 |
| I-623 | I-666 | I-709 |
| I-624 | I-667 | I-710 |
| I-625 | I-670 | I-711 |
| I-626 | I-671 | I-713 |
| I-627 | I-677 | I-714 |
| I-628 | I-678 | I-715 |
| I-635 | I-679 | I-716 |
| I-636 | I-681 | I-717 |
| I-637 | I-682 | I-718 |
| I-640 | I-683 | I-719 |
| I-641 | I-684 | I-720 |
| I-642 | I-685 | I-721 |
| I-643 | I-686 | I-722 |
| I-644 | I-687 | I-723 |
| I-645 | I-692 | I-724 |
| I-646 | I-693 | I-726 |
| I-647 | I-694 | I-728 |
| I-729 | I-743 | I-758 |
| I-730 | I-744 | I-759 |
| I-731 | I-745 | I-760 |
| I-732 | I-746 | I-761 |
| I-733 | I-747 | I-762 |
| I-734 | I-749 | I-763 |
| I-735 | I-750 | I-765 |
| I-736 | I-751 | I-766 |
| I-737 | I-752 | I-768 |
| I-738 | I-753 | I-775 |
| I-739 | I-754 | I-809 |
| I-740 | I-755 | I-810 |
| I-741 | I-756 | I-811 |
| I-742 | I-757 | |

### Example 5

### 6-[(2-chloro-5-fluorophenyl)amino]-N-methyl-5-({[(3r,5s)-adamantan-1-yl]carbamoyl}amino)pyridine-3-carboxamide (I-799)

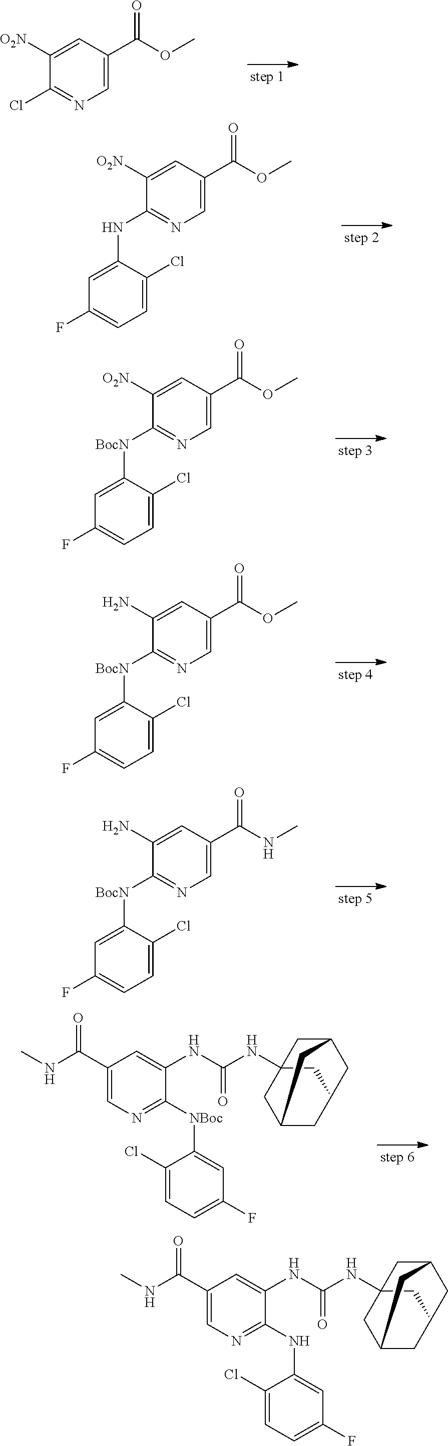

### Step 1. methyl 6-[(2-chloro-5-fluorophenyl)amino]-5-nitropyridine-3-carboxylate

To a stirred solution of methyl 6-chloro-5-nitropyridine-3-carboxylate (2.16 g, 9.973 mmol, 1.00 equiv) and 2-chloro-5-fluoroaniline (1.45 g, 9.973 mmol, 1.00 equiv), p-Toluenesulfonic acid (1.72 g, 9.991 mmol, 1.00 equiv) in 1,4-dioxane (20 mL). The resulting mixture was stirred for 3 h at 180° C. under nitrogen atmosphere with microwave. The reaction was quenched with sat. NH₄Cl (aq.) at room temperature. The resulting mixture was extracted with EtOAc (3×200 mL). The combined organic layers were washed with brine (3×100 mL), dried over anhydrous Na2SO4. After filtration, the filtrate was concentrated under reduced pressure. The residue was purified by silica gel column chromatography, eluted with CH2Cl2/MeOH (100:1) to afford methyl 6-[(2-chloro-5-fluorophenyl)amino]-5-nitropyridine-3-carboxylate (1.63 g, 50.18%) as a white solid. Lcms: Rt=0.823 min, m/z=326.05 (M+1)+.

### Step 2. methyl 6-[(tert-butoxycarbonyl)(2-chloro-5-fluorophenyl)amino]-5-nitropyridine-3-carboxylate

To a stirred solution of methyl 6-[(2-chloro-5-fluorophenyl)amino]-5-nitropyridine-3-carboxylate (3.25 g, 9.979 mmol, 1.00 equiv) and DMAP (1.22 g, 9.979 mmol, 1.00 equiv) in THF was added Boc2O (3.27 g, 14.969 mmol, 1.50 equiv) dropwise at room temperature under air atmosphere. The resulting mixture was stirred for 30 min at 80° C. under air atmosphere. The resulting mixture was concentrated under vacuum. The residue was purified by silica gel column chromatography, eluted with hexane/EtOAc (6:1) to afford methyl 6-[(tert-butoxycarbonyl)(2-chloro-5-fluorophenyl)amino]-5-nitropyridine-3-carboxylate (3.3 g, 77.66%) as a yellow solid. Lcms: Rt=0.824 min, m/z=370.00 (M+1-56)+.

### Step 3. methyl 5-amino-6-[(tert-butoxycarbonyl)(2-chloro-5-fluorophenyl)amino]pyridine-3-carboxylate

To a stirred solution of methyl 6-[(tert-butoxycarbonyl)(2-chloro-5-fluorophenyl)amino]-5-nitropyridine-3-carboxylate (4.25 g, 1.00 equiv) in ethyl acetate (40 mL) was added Pd/C (0.4 g) in one portion at room temperature under nitrogen atmosphere. The resulting mixture was stirred for 1 day at room temperature under hydrogen atmosphere. The resulting mixture was filtered, the filter cake was washed with MeOH (3×50 mL). The filtrate was concentrated under reduced pressure to get methyl 5-amino-6-[(tert-butoxycarbonyl)(2-chloro-5-fluorophenyl)amino]pyridine-3-carboxylate (2.3 g, 58%) as a yellow solid. Lcms: Rt=0.753 min, m/z=396.10 (M+1)+

### Step 4. tert-butyl N-(2-chloro-5-fluorophenyl)-N-[5-(methylcarbamoyl)-3-nitropyridin-2-yl]carbamate

To a stirred solution of methyl 5-amino-6-[(tert-butoxycarbonyl)(2-chloro-5-fluorophenyl)amino]pyridine-3-carboxylate (395 mg, 0.998 mmol, 1.00 equiv) in MeOH (10 mL) was added methylamine (10 mL, 9.98 mmol, 10.00 equiv) dropwised at room temperature under nitrogen atmosphere. The resulting mixture was stirred for 1 day at room temperature under nitrogen atmosphere. The resulting mixture was filtered, the filter cake was washed with MeOH (3×50 mL). The filtrate was concentrated under reduced pressure to get tert-butyl N-(2-chloro-5-fluorophenyl)-N-[5-(methylcarbamoyl)-3-nitropyridin-2-yl]carbamate (350 g, 88%) as a yellow solid. Lcms: Rt=0.763 min, m/z=295.05 (M+1-100)+

### Step 5. tert-butyl N-(2-chloro-5-fluorophenyl)-N-[5-(methylcarbamoyl)-3-({[(3r)-adamantan-1-yl]carbamoyl}amino)pyridin-2-yl]carbamate

To a solution of tert-butyl N-[3-amino-5-(methylcarbamoyl)pyridin-2-yl]-N-(2-chloro-5-fluorophenyl)carbamate (200 mg, 506 μmol) and TEA (152 mg, 3 eq) in THF (2 mL) was added ditrichloromethyl carbonate (59.9 mg, 0.4 eq). After stirring at 0° C. for 1.5 h, (3R,5S,7s)-adamantan-1-amine (152 mg, 1.01 mmol) was added and the resulting mixture was stirred at room temperature for 1 h. The resulting mixture was concentrated under reduced pressure and was dissolved in DMF. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in tert-butyl N-(2-chloro-5-fluorophenyl)-N-[5-(methylcarbamoyl)-3-({[(3r)-adamantan-1-yl]carbamoyl}amino)pyridin-2-yl]carbamate (80 mg, 139 μmol, 27.7%) as a white solid. Lcms: Rt=1.357 min, m/z=572.45 (M+1)+

### Step 6. 6-[(2-chloro-5-fluorophenyl)amino]-N-methyl-5-({[(3r,5s)-adamantan-1-yl]carbamoyl}amino)pyridine-3-carboxamide

To a solution of tert-butyl N-(2-chloro-5-fluorophenyl)-N-[5-(methylcarbamoyl)-3-({[(3r)-adamantan-1-yl]carbamoyl}amino)pyridin-2-yl]carbamate (50 mg, 87.4 μmol) in DCM (0.5 mL) was added TFA (0.5 mL). The reaction mixture was stirred at room temperature for 2 h. The resulting mixture was concentrated under reduced pressure and was dissolved in DMF. The resulted solution was purified using prep-HPLC with following conditions: Column: YMC-Actus Triart C18, 30*250, 5 um; Mobile Phase A: Water (10 mmol/L NH4HCO3), Mobile Phase B: ACN; Flow rate: 60 mL/min; Gradient: 55% B to 85% B in 7 min; 254/220 nm; RT: 6.2 min. This resulted in 6-[(2-chloro-5-fluorophenyl)amino]-N-methyl-5-({[(3r,5s)-adamantan-1-yl]carbamoyl}amino)pyridine-3-carboxamide (21.0 mg, 44.4 μmol, 50.9%) as a white solid. Lcms: Rt=1.092 min, m/z=472.25 (M+1)+. 1H NMR (400 MHz, DMSO-d6): 8.51 (2H, dd, J=16.9, 1.8 Hz), 8.42 (1H, q, J=4.4 Hz), 8.13 (1H, s), 8.08-7.99 (2H, m), 7.49 (1H, dd, J=8.9, 6.0 Hz), 6.84 (1H, ddd, J=8.8, 7.9, 3.0 Hz), 6.35 (1H, s), 2.78 (3H, d, J=4.5 Hz), 2.04 (3H, s), 1.97 (6H, d, J=2.9 Hz), 1.64 (6H, d, J=3.0 Hz).

Additional compounds prepared according to the methods of Example 5 are listed in Table 5 below. Corresponding ¹H NMR and mass spectrometry characterization for these compounds are described in Table 1. Certain compounds in Table 3 below were prepared with other compounds whose preparation is described further in the Examples herein.

TABLE 5
Additional Compounds
I-798

### Example 6

### 5-((2-chloro-5-fluorophenyl)amino)-6-(3-fluoro-5-(trifluoromethyl)benzamido)-N-methylpicolinamide (I-803)

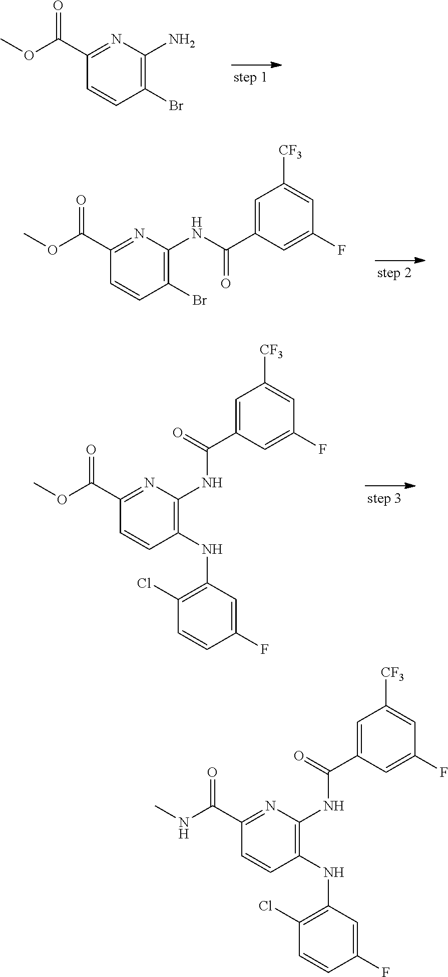 [SMILES: COC(=O)c1ccc(Br)c(N)n1 ; COC(=O)c1ccc(Br)c(NC(=O)c2cc(F)cc(C(F)(F)F)c2)n1 ; COC(=O)c1ccc(Nc2cc(F)ccc2Cl)c(NC(=O)c2cc(F)cc(C(F)(F)F)c2)n1 ; CNC(=O)c1ccc(Nc2cc(F)ccc2Cl)c(NC(=O)c2cc(F)cc(C(F)(F)F)c2)n1]

### Step 1. methyl 5-bromo-6-(3-fluoro-5-(trifluoromethyl)benzamido)picolinate

A round bottomed flask was charged with methyl 6-amino-5-bromopyridine-2-carboxylate (1 g, 4.32 mmol), 3-fluoro-5-(trifluoromethyl)benzoic acid (988 mg, 4.75 mmol), 4-methylmorpholine (654 mg, 1.5 eq), HBTU (4.94 g, 3.0 eq) and a stirbar. N,N-dimethylformamide (20 mL) was added, and the solution was stirred at 50° C. for 5 h. The reaction was quenched with water and extracted with EA. The organic layer was washed with brine, dried over Na2SO4 and evaporated. The organic layer was dried over Na2SO4 and evaporated to dryness. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). methyl 5-bromo-6-[3-fluoro-5-(trifluoromethyl)benzamido]pyridine-2-carboxylate (2.2 g, 5.22 mmol, 121%). m/z (ES+) [M+H]+=423.05; HPLC tR=0.924 min.

### Step 2. methyl 5-((2-chloro-5-fluorophenyl)amino)-6-(3-fluoro-5-(trifluoromethyl)benzamido)picolinate

Methyl 5-bromo-6-[3-fluoro-5-(trifluoromethyl)benzamido]pyridine-2-carboxylate (500 mg, 1.18 mmol), Pd(OAc)₂ (13.3 mg, 0.05 eq), Cs₂(CO₃) (462 mg, 1.2 eq), and X-PHOS (17.85 mg) were added to a screw-cap vial. The vial was fitted with a rubber septum, evacuated, and backfilled with N₂. 2-chloro-5-fluoroaniline (179 mg, 1.23 mmol) was injected into the vial with a syringe under a positive pressure of argon. Toluene (20 mL) was added via a syringe. The rubber septum was replaced with a screw-cap, and the sealed vial was introduced into a preheated oil bath at 100° C. After 15 h the reaction mixture was filtered through a short pad of Celite, washed with water and brine, dried (Na2SO4), and concentrated under reduced pressure. The resulted solution was purified using C18 flash chromatography with the following conditions (Mobile Phase A: Water, Mobile Phase B: ACN; Flow rate: 30 mL/min; Gradient: 0% B to 100% B in 40 min; 254/220 nm). This resulted in methyl 5-[(2-chloro-5-fluorophenyl)amino]-6-[3-fluoro-5-(trifluoromethyl)benzamido]pyridine-2-carboxylate (450 mg, 926 μmol, 78.5%) as a yellow solid. m/z (ES+) [M+H]=486.10; HPLC tR=0.977 min.

### Step 3. 5-((2-chloro-5-fluorophenyl)amino)-6-(3-fluoro-5-(trifluoromethyl)benzamido)-N-methylpicolinamide

A round bottomed flask was charged with methyl 5-[(2-chloro-5-fluorophenyl)amino]-6-[3-fluoro-5-(trifluoromethyl)benzamido]pyridine-2-carboxylate (50 mg, 102 μmol), methanamine (633 mg, 200 eq) solution in water (18 mL) and a stir bar. MeOH (30 mL) was added, and the solution was stirred at room temperature for 5 h. The solvents were evaporated in vacuum. Adjusted to pH=7 with dilute hydrochloric acid, then the mixture was extracted three times with ethyl acetate. The combined organic layers were dried over Na₂SO₄, and the solvents were evaporated in vacuum. A solution of 5-[(2-chloro-5-fluorophenyl)amino]-6-[3-fluoro-5-(trifluoromethyl)benzamido]-N-methylpyridine-2-carboxamide (1, 52.00 mg, 0.11 mol) was purified using prep-HPLC with following conditions: Column: XBridge Prep OBD C18 Column, 30×150 mm 5 um; Mobile Phase A: Water (10 MMOL/L NH4HCO3), Mobile Phase B: ACN; Flow rate: 60 mL/min; Gradient: 30% B to 55% B in 8 min; 254/220 nm; RT1: 7.55. Lyophilization yielded 5-[(2-chloro-5-fluorophenyl)amino]-6-[3-fluoro-5-(trifluoromethyl)benzamido]-N-methylpyridine-2-carboxamide (20.9 mg, 43.1 μmol, 42.3%) as a yellow amorphous solid. m/z (ES+) [M+H]+=485.25; HPLC tR=1.659 min. ¹H NMR (Chloroform-d, 400 MHz) δ 8.64 (1H, s), 8.14-8.05 (2H, m), 8.00-7.93 (1H, m), 7.89 (1H, d, J=8.4 Hz), 7.71 (1H, s), 7.61 (2H, d, J=7.6 Hz), 7.35 (1H, dd, J=8.8, 5.7 Hz), 6.81 (1H, dd, J=10.2, 2.8 Hz), 6.64 (1H, ddd, J=8.8, 7.7, 2.8 Hz), 3.03 (3H, d, J=5.0 Hz).

### Example 7

### 3-fluoro-N-(((2R,3S)-2-(o-tolyl)indolin-3-yl)methyl)-5-(trifluoromethyl)benzamide (I-808)

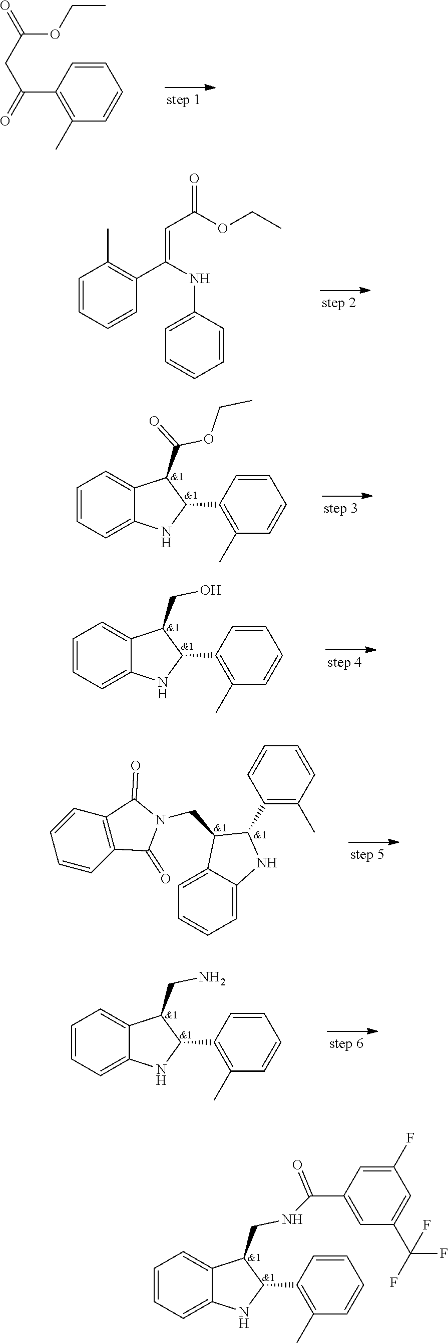

### Step 1. ethyl (Z)-3-(phenylamino)-3-(o-tolyl)acrylate

A round bottomed flask was charged with ethyl 3-(2-methylphenyl)-3-oxopropanoate (3 g, 14.5 mmol), aniline (6.75 g, 72.5 mmol), acetic acid (4.35 g, 5.0 eq) and a stirbar, and the solution was stirred at 80° C. The pH of the resulted solution was adjusted to 7 with NaHCO3 aq. The solution was extracted with EA. The organic layer was dried over Na2SO4 and evaporated to dryness to afford ethyl (2Z)-3-(2-methylphenyl)-3-(phenylamino)prop-2-enoate (4 g, 14.2 mmol, 98.2%) as a yellow solid. m/z (ES+) [M+H]+=282; HPLC tR=0.856 min.

### Step 2. ethyl 2-(o-tolyl)indoline-3-carboxylate

A round bottomed flask was charged with ethyl (2Z)-3-(2-methylphenyl)-3-(phenylamino)prop-2-enoate (564 mg, 2.00 mmol)), tris(2-phenylpyridine) iridium (26.3 mg, 0.02 eq) and a stirbar. THF (30 mL) was added, and the solution was stirred under 450 nm blue light for 72 h. The resulting mixture was concentrated under reduced pressure and was dissolved in DMF. The resulted solution was purified using C18 flash chromatography with the following conditions (Mobile Phase A: Water, Mobile Phase B: ACN; Flow rate: 20 mL/min; Gradient: 0% B to 100% B in 40 min; 254/220 nm). This resulted in ethyl (2R,3R)-2-(2-methylphenyl)-2,3-dihydro-1H-indole-3-carboxylate (100 mg, 355 μmol, 20.1%) as a yellow oily. m/z (ES+) [M+H]+282.15; HPLC tR=2.154 min.

### Step 3. (2-(o-tolyl)indolin-3-yl)methanol

A round bottomed flask w as charged with ethyl 2-(2-methylphenyl)-2,3-dihydro-1H-indole-3-carboxylate (100 mg, 355 μmol) and a stirbar. EtOH (10 mL) was added, then NaBH4 (26.8 mg, 710 μmol) was added to solution, and the solution was stirred at 25° C. overnight. The reaction mixture was diluted with satd. NH4Cl (100 mL), and the aqueous phase was extracted with EA (50 ml) three times. The combined organic layers were washed with Sat NaCl, dried over sodium sulfate, filtered, and concentrated in vacuo. The resulting crude material was purified by silica gel chromatography (10 g column; eluting with heptanes/ethyl acetate; 1:1). Concentration in vacuo resulted in [2-(2-methylphenyl)-2,3-dihydro-1H-indol-3-yl]methanol (50.0 mg, 208 μmol) as a white solid, m/z (ES+) [M+H]+=240.1; HPLC tR=0.647 min.

### Step 4. 2-((2-(o-tolyl)indolin-3-yl)methyl)isoindoline-1,3-dione

A resealable reaction vial was charged with [2-(2-methylphenyl)-2,3-dihydro-1H-indol-3-yl]methanol (112 mg, 467 μmol), THF (1 mL), DIAD (10 mg, 0.396 eq) was added and a stirbar before being evacuated and purged with nitrogen three time. 2,3-dihydro-1H-isoindole-1,3-dione (82.3 mg, 560 μmol) was added and Ph₃P (16.3 mg, 1 eq) in THF (0.1 mL) was dropped in the mixture at 0° C. The mixture was stirred at 25° C. for overnight. The reaction was quenched with water and extracted with EA. The organic layer was washed with brine, dried over Na₂SO₄ and evaporated. This resulted in 2-{[2(2-methylphenyl)-2,3-dihydro-1H-indol-3-yl]methyl}-2,3-dihydro-1H-isoindole-1,3-dione (180 mg, 488 μmol, 104%) as a yellow solid. m/z (ES+) [M+H]+=263.15; HPLC tR=1.292 min.

### Step 5. (2-(o-tolyl)indolin-3-yl)methanamine

A round bottomed flask was charged with 2-{[2-(2-methylphenyl)-2,3-dihydro-1H-indol-3-yl]methyl}-2,3-dihydro-1H-isoindole-1,3-dione (180 mg, 488 μmol), hydrazine hydrate (95.6 mg, 4 eq), ethanol (179 mg, 8 eq) and a stirbar. The solution was stirred at 25° C. The resulting mixture was concentrated under reduced pressure. The solution was adjusted to pH 5˜6 with HCl (1 mol/L), and the solution was washed with EA. Then the solution was adjusted to pH 7˜8 with NaHCO3 (saturated solution), and the solution was extracted with EA. The organic layer was dried over Na2SO4 and evaporated to dryness to afford 1-[2-(2-methylphenyl)-2,3-dihydro-1H-indol-3-yl]methanamine (100 mg, 419 μmol, 86.2%) as a yellow oil. m/z (ES+) [M+H]+=239.15; HPLC tR=0.539 min.

### Step 6. 3-fluoro-N-((2-(o-tolyl)indolin-3-yl)methyl)-5-(trifluoromethyl)benzamide

A round bottomed flask was charged with 3-fluoro-5-(trifluoromethyl)benzoic acid (21.6 mg, 104 μmol), Ghosez reagent (16.7 mg, 0.6 eq) and a stirbar. DCM (3 mL) was added, and the solution was stirred at 25° C. The round bottomed flask was charged with TEA (31.6 mg, 1.5 eq), 1-[2-(2-methylphenyl)-2,3-dihydro-1H-indol-3-yl]methanamine (50 mg, 209 μmol) and the solution was stirred at 25° C. The solution was washed with Saturated sodium bicarbonate solution and Saturated salt water. The organic layer was dried over Na2SO4 and evaporated to dryness. A solution of 3-fluoro-N-{[2-(2-methylphenyl)-2,3-dihydro-1H-indol-3-yl]methyl}-5-(trifluoromethyl)benzamide (1, 30.00 mg, 0.07 mmol) was purified using prep-HPLC with following conditions: Column: XBridge Prep OBD C18 Column, 30×150 mm 5 um; Mobile Phase A: Water (10 MMOL/L NH4HCO3+0.1% NH3·H2O), Mobile Phase B: ACN; Flow rate: 60 mL/min; Gradient: 50% B to 80% B in 7 min; 220 nm; RT1: 6.15. Lyophilization yielded 3-fluoro-N-{[2-(2-methylphenyl)-2,3-dihydro-1H-indol-3-yl]methyl}-5-(trifluoromethyl)benzamide (16.8 mg, 39.2 μmol, 18.7%) as a white amorphous solid. m/z (ES+) [M+H]+=429.20; HPLC tR=1.926 min. ¹H NMR (400 MHz, Chloroform-d) δ 7.59 (s, 1H), 7.47 (dd, J=17.1, 8.3 Hz, 2H), 7.31 (d, J=74 Hz, 1H), 7.24-7.11 (m, 5H), 6.87-6.78 (m, 2H), 6.31 (s, 1H), 4.97 (d, J=4.7 Hz, 1H), 4.02 (dt, J=13.5, 5.4 Hz, 1H), 2.47 (s, 3H), 3.83 (dt, J=13.5, 5.5 Hz, 1H), 3.59 (q, J=5.2 Hz, 1H).

### Example 8

### N-{[(2R,3S)-2-(2-chloro-5-fluorophenyl)-5-oxopyrrolidin-3-yl]methyl}-3-fluoro-5-(trifluoromethyl)benzamide (I-801)

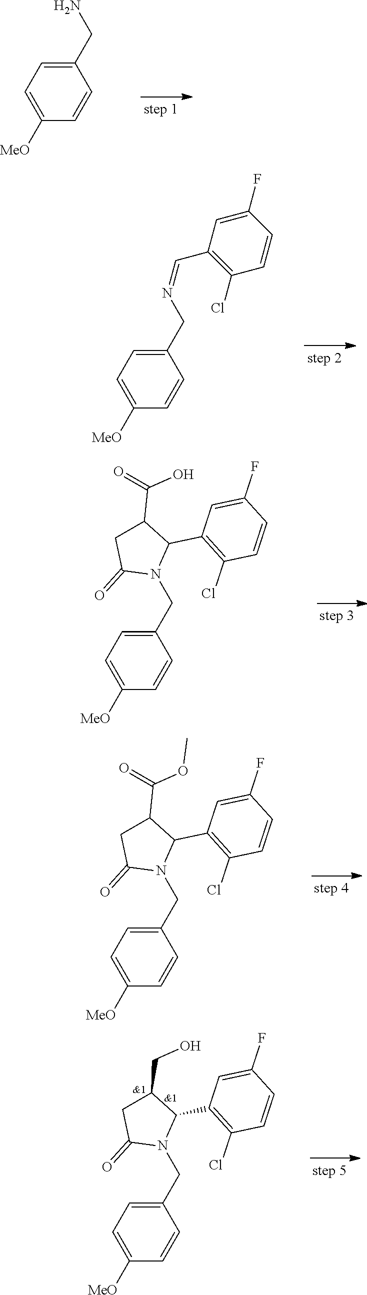

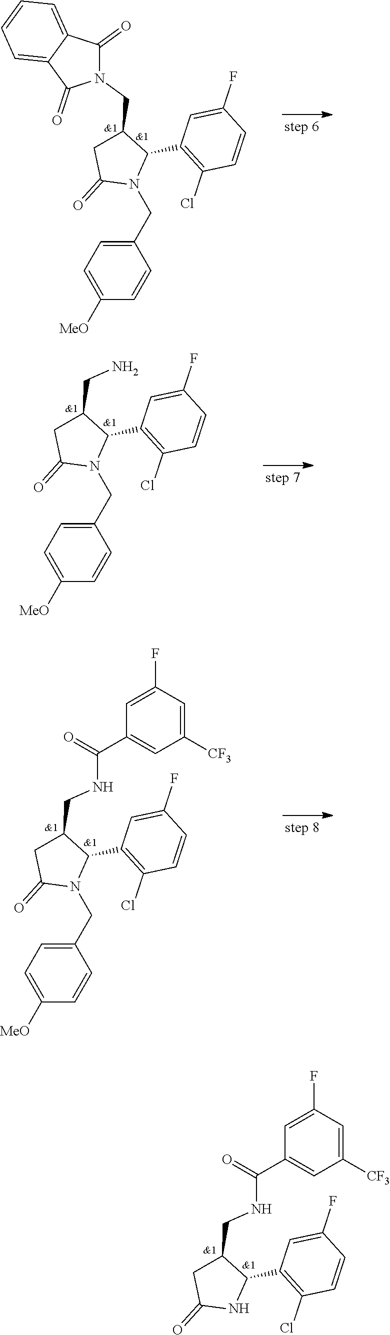 [SMILES: COc1ccc(CN2C(=O)C[C@@H](CN3C(=O)c4ccccc4C3=O)[C@@H]2c2cc(F)ccc2Cl)cc1 ; COc1ccc(CN2C(=O)C[C@@H](CN)[C@@H]2c2cc(F)ccc2Cl)cc1 ; COc1ccc(CN2C(=O)C[C@@H](CNC(=O)c3cc(F)cc(C(F)(F)F)c3)[C@@H]2c2cc(F)ccc2Cl)cc1 ; O=C1C[C@@H](CNC(=O)c2cc(F)cc(C(F)(F)F)c2)[C@H](c2cc(F)ccc2Cl)N1]

### Step 1. (Z)-[(2-chloro-5-fluorophenyl)methylidene][(4-methoxyphenyl)methyl]amine

To a solution of 1-(4-methoxyphenyl)methanamine (2 g, 14.5 mmol) and 2-chloro-5-fluorobenzaldehyde (2.29 g, 1 eq) in DCM (20 mL) was added MgSO4 (3.48 g, 2 eq). The reaction mixture was stirred at room temperature for 12 h. The resulting mixture was filtered, the filter cake was washed with DCM. The filtrate was concentrated under reduced pressure. This resulted in (Z)-[(2-chloro-5-fluorophenyl)methylidene][(4-methoxy phenyl)methyl]amine (2.6 g, 9.36 mmol, 64.6%) as a white solid, Lcms: Rt=1.459 min, m/z=278.05 (M+1)+.

### Step 2. 2-(2-chloro-5-fluorophenyl)-1-[(4-methoxyphenyl)methyl]-5-oxopyrrolidine-3-carboxylic Acid

To a solution of (Z)-[(2-chloro-5-fluorophenyl)methylidene][(4-methoxyphenyl)methyl]amine (1.2 g, 4.32 mmol) in xylene (10 mL) was added oxolane-2,5-dione (432 mg, 1 eq). The reaction mixture was stirred at 140° C. for 12 h. The resulting mixture was concentrated under reduced pressure and was dissolved in DMF. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in 2-(2-chloro-5-fluorophenyl)-1-[(4-methoxyphenyl)methyl]-5-oxopyrrolidine-3-carboxylic acid (1.8 g, 4.76 mmol, crude) as a white solid. LCMS: Rt=0.819 min, m/z=378.15 (M+1)+.

### Step 3. methyl 2-(2-chloro-5-fluorophenyl)-1-[(4-methoxyphenyl)methyl]-5-oxopyrrolidine-3-carboxylate

To a stirred solution of 2-(2-chloro-5-fluorophenyl)-1-[(4-methoxyphenyl)methyl]-5-oxopyrrolidine-3-carboxylic acid (1.6 g, 4.23 mmol) and K2CO3 (1.73 g, 3 eq) in acetone (16 mL) was added MeI (1.73 g, 3 eq) at 0° C. The resulting mixture was stirred at room temperature for 16 h. The reaction was quenched with water and extracted with EA. The organic layer was washed with brine, dried over Na2SO4 and evaporated. The residue was purified by Prep-TLC to afford methyl 2-(2-chloro-5-fluorophenyl)-1-[(4-methoxyphenyl)methyl]-5-oxopyrrolidine-3-carboxylate (700 mg, 1.78 mmol, 42.4%) as a light yellow solid. LCMS: Rt=0.860 min, m/z=392.10 (M+1)+.

### Step 4. 5-(2-chloro-5-fluorophenyl)-4-(hydroxymethyl)-1-[(4-methoxyphenyl)methyl]pyrrolidin-2-one

To a stirred solution of methyl 2-(2-chloro-5-fluorophenyl)-1-[(4-methoxyphenyl)methyl]-5-oxopyrrolidine-3-carboxylate (650 mg, 1.65 mmol) in EtOH (7 mL) was added NaBH4 (627 mg, 10 eq) at 0° C. The resulting mixture was stirred at room temperature for 12 h. The reaction was quenched with water and extracted with EA. The organic layer was washed with brine, dried over Na2SO4 and evaporated. The residue was purified by Prep-TLC. This resulted in 5-(2-chloro-5-fluorophenyl)-4-(hydroxymethyl)-1-[(4-methoxyphenyl)methyl]pyrrolidin-2-one (400 mg, 1.09 mmol, 66.6%) as a white solid. Lcms: Rt=0.632 min, m/z=364.10 (M+1)+.

### Step 5. 2-{[2-(2-chloro-5-fluorophenyl)-1-[(4-methoxyphenyl)methyl]-5-oxopyrrolidin-3-yl]methyl}-2,3-dihydro-1H-isoindole-1,3-dione

To a stirred solution of 5-(2-chloro-5-fluorophenyl)-4-(hydroxymethyl)-1-[(4-methoxyphenyl)methyl]pyrrolidin-2-one (300 mg, 824 μmol), PPh3 (429 mg, 2 eq) and 2,3-dihydro-1H-isoindole-1,3-dione (145 mg, 985 μmol) in THF (3 mL) was added DIAD (0.32 mL) in THF (1 mL) at 0° C. The resulting mixture was stirred at room temperature overnight. The resulting mixture was concentrated under reduced pressure. This resulted in 2-{[2-(2-chloro-5-fluorophenyl)-1-[(4-methoxyphenyl)methyl]-5-oxopyrrolidin-3-yl]methyl}-2,3-dihydro-1H-isoindole-1,3-dione (350 mg, 710 μmol, 86.2%) as a light yellow solid. Lcms: Rt=1.235 min, m/z=493.30 (M+1)+.

### Step 6. 4-(aminomethyl)-5-(2-chloro-5-fluorophenyl)-1-[(4-methoxyphenyl)methyl]pyrrolidin-2-one

To a solution of 2-{[2-(2-chloro-5-fluorophenyl)-1-[(4-methoxyphenyl)methyl]-5-oxopyrrolidin-3-yl]methyl}-2,3-dihydro-1H-isoindole-1,3-dione (300 mg, 608 μmol) in EtOH (3 mL) was added N2H4·H2O (1.5 mL). The reaction mixture was stirred at 60° C. for 12 h. The solution was extracted with EA The organic layer was dried over Na2SO4 and evaporated to dryness. This resulted in 4-(aminomethyl)-5-(2-chloro-5-fluorophenyl)-1-[(4-methoxyphenyl)methyl]pyrrolidin-2-one (200 mg, 551 μmol, 90.9%) as a white solid. Lcms: Rt=0.910 min, m/z=363.20 (M+1)+.

### Step 7. N-{[2-(2-chloro-5-fluorophenyl)-1-[(4-methoxyphenyl)methyl]-5-oxopyrrolidin-3-yl]methyl}-3-fluoro-5-(trifluoromethyl)benzamide

To a stirred solution of 4-(aminomethyl)-5-(2-chloro-5-fluorophenyl)-1-[(4-methoxyphenyl)methyl]pyrrolidin-2-one (180 mg, 496 μmol), 3-fluoro-5-(trifluoromethyl)benzoic acid (154 mg, 744 μmol) and NaHCO3 (124 mg, 3 eq) in DMF (2.0 mL) was added HATU (282 mg, 1.5 eq) at room temperature. The resulting mixture was stirred at room temperature for 1 h. The reaction was quenched with water and extracted with EA. The organic layer was washed with brine, dried over Na2SO4 and evaporated. The resulting mixture was concentrated under reduced pressure and was dissolved in DMF. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in N-{[2-(2-chloro-5-fluorophenyl)-1-[(4-methoxyphenyl)methyl]-5-oxopyrrolidin-3-yl]methyl}-3-fluoro-5-(trifluoromethyl)benzamide (150 mg, 271 μmol, 54.7%) as a white solid. Lcms: Rt=0.991 min, m/z=553.30 (M+1)+.

### Step 8. N-{[2-(2-chloro-5-fluorophenyl)-5-oxopyrrolidin-3-yl]methyl}-3-fluoro-5-(trifluoromethyl)benzamide

To N-{[2-(2-chloro-5-fluorophenyl)-1-[(4-methoxyphenyl)methyl]-5-oxopyrrolidin-3-yl]methyl}-3-fluoro-5-(trifluoromethyl)benizamide (130 mg, 235 μmol) in ACN (2 mL), was added 1.0 M CAN in 0.04 ml water and another 2 ml CH3CN (to keep a 1:100 water/CH3CN ratio). The mixture was stirred 2 h, then 6 ml CH2Cl2 were added and stirring was continued a further 12 h. Solids were next filtered off, and the reaction mixture was concentrated in vacuo at r.t. The residue was taken up in 100 ml ethyl ether, and solid NaHCO3 was added until the brown precipitate turned deep yellow. After separation of the solids, the mixture was worked up as usual. The residue was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in N-{[2-(2-chloro-5-fluorophenyl)-5-oxopyrrolidin-3-yl]methyl}-3-fluoro-5-(trifluoromethyl)benzamide (30.1 mg, 69.5 μmol, 29.8%) as a white solid. Lcms: Rt=0.922 min, m/z=433.30 (M+1)+. ¹H NMR (400 MHz, DMSO-d6) δ 9.04 (1H, t, J=5.9 Hz), 8.17 (1H, s), 8.02 (1H, s), 7.92 (2H, td, J=9.0, 8.3, 2.2 Hz), 7.49 (1H, dd, J=8.8, 5.1 Hz), 7.19 (1H, td, J=8.4, 3.1 Hz), 7.11 (1H, dd, J=9.7, 3.1 Hz), 4.79 (1H, s), 3.55 (1H, dt, J=13.0, 6.3 Hz), 3.45 (1H, dt, J=13.1, 6.0 Hz), 2.50-2.43 (2H, m) 2.12-2.01 (1H, m).

### Example 9

### N-{[(2R,3S)-2-(2-chloro-5-fluorophenyl)-5-oxopyrrolidin-3-yl]methyl}-3-fluoro-5-(trifluoromethyl)benzamide

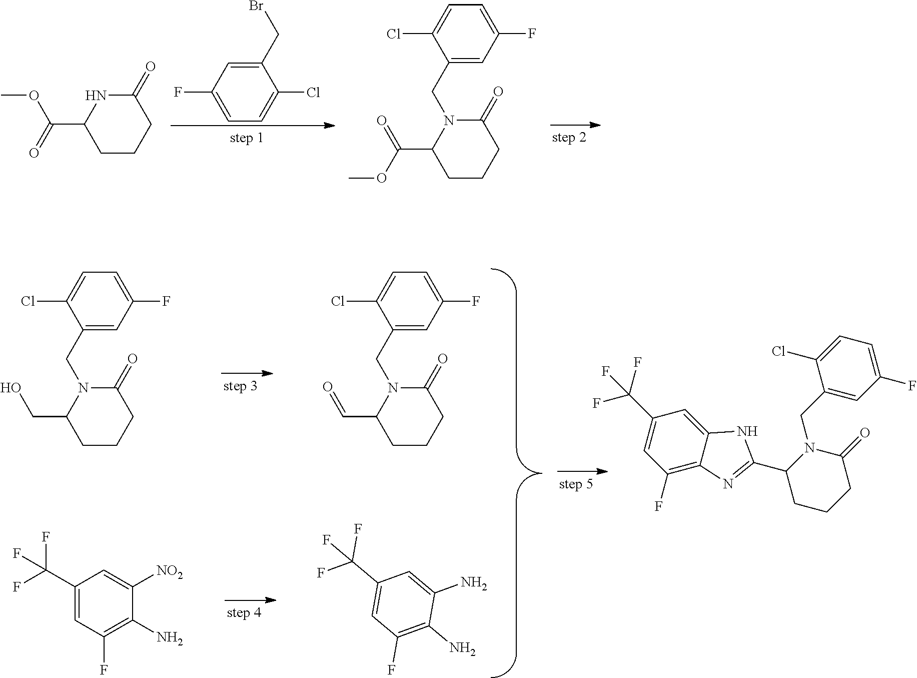

### Step 1. methyl 1-[(2-chloro-5-fluorophenyl)methyl]-6-oxopiperidine-2-carboxylate

Sodium hydride (83.7 mg, 1.1 eq) was added to a solution of the methyl 6-oxopiperidine-2-carboxylate (500 mg, 3.18 mmol) in anhydrous DMF at 0° C. under N2. After 10 min, 2-(bromomethyl)-1-chloro-4-fluorobenzene (779 mg, 3.49 mmol) was added, and the reaction mixture was stirred at room temperature. After 4 h, saturated NH4Cl (10 mL) was added and the mixture was extracted several times with brine, dried over anhydrous Na2SO4, filtered and evaporated to give a residue, which was chromatographed (eluent: PE/EA 5/1) to afford methyl 1-[(2-chloro-5-fluorophenyl)methyl]-6-oxopiperidine-2-carboxylate (380 mg, 1.26 mmol, 39.8%) as a white solid. Lcms: Rt=0.661 min, m/z=300.05 (M+1)+.

### Step 2. 2-(2-chloro-5-fluorophenyl)-1-[(4-methoxyphenyl)methyl]-5-oxopyrrolidine-3-carboxylic Acid

To a solution of methyl 1-[(2-chloro-5-fluorophenyl)methyl]-6-oxopiperidine-2-carboxylate (300 mg, 1.00 mmol) in anhydrous ethanol (20 mL), sodium borohydride (90.7 mg, 2.4 eq) was added in portions over 5 minutes. The solution was stirred for 3.5 hours at room temperature. The mixture was then treated with glacial acetic acid (336 mg, 5.6 eq) and the mixture was then concentrated in vacuo and the resulting oil solidified upon standing under vacuum. The crude product was dissolved in DCM (20 mL), washed with Saturated sodium bicarbonate solution and the resulting was concentrated in vacuo. This resulted in 1-[(2-chloro-5-fluorophenyl)methyl]-6-(hydroxymethyl)piperidin-2-one (250 mg, 920 μmol, 92.2%) as a white solid. Lcms: Rt=0.697 min, m/z=272.05 (M+1)+.

### Step 3. 2-(2-chloro-5-fluorophenyl)-1-[(4-methoxyphenyl)methyl]-5-oxopyrrolidine-3-carboxylic Acid

A solution of 1-[(2-chloro-5-fluorophenyl)methyl]-6-(hydroxymethyl)piperidin-2-one (600 mg, 2.20 mmol) in dichloromethane (40 mL) chilled in an ice-water bath was treated with Dess-Martin's reagent (1.86 g, 2 eq). After 1 h, TLC analysis indicated complete consumption of starting material. The mixture was stirred for 5 min with 10% aqueous sodium thiosulfate solution and poured into a separatory funnel. The organic phase was washed once more with 10% sodium thiosulfate followed by saturated aqueous sodium bicarbonate (2×), water, and brine; dried (Na2SO4); filtered; and concentrated to afford 1-[(2-chloro-5-fluorophenyl)methyl]-6-oxopiperidine-2-carbaldehyde (220 mg, 815 μmol, 37.0%) as an off-white amorphous solid. Lcms: Rt=0.618 min, m/z=302.05 (M+1)+.

### Step 4. N-{[(2R,3S)-2-(2-chloro-5-fluorophenyl)-1-[(4-methoxyphenyl)methyl]-5-oxopyrrolidin-3-yl]methyl}-3-fluoro-5-(trifluoromethyl)benzamide

2-fluoro-6-nitro-4-(trifluoromethyl)aniline (2 g, 8.92 mmol) and methanol (20 mL) were added to a 100 mL round bottom flask, which was evacuated and refilled with N2 for 3 times. After the addition, the Pd/C (200 ng) was added to the flask which was evacuated and refilled with N2 for 3 times, sealed tube was placed at room temperature, under H2, and stirred for 3 h. After completion, the resulting mixture was filtered, the filter cake was washed with MeOH. The filtrate was concentrated under reduced pressure. This resulted in 3-fluoro-5-(trifluoromethyl)benzene-1,2-diamine (1.85 g, 9.52 mmol, crude) as a black oil. Lcms: Rt=0.894 min, m/z=195.05 (M+1)+

### Step 5. 1-[(2-chloro-5-fluorophenyl)methyl]-6-[4-fluoro-6-(trifluoromethyl)-1H-1,3-benzodiazol-2-yl]piperidin-2-one

1-[(2-chloro-5-fluorophenyl)methyl]-6-oxopiperidine-2-carbaldehyde (240 mg, 889 μmol) and 3-fluoro-5-(trifluoromethyl)benzene-1,2-diamine (172 mg, 889 μmol) were dissolved in N,N-dimethylformamide (3 mL). To this solution was added water (320 μL) followed by [(hydroperoxysulfonyl)oxy]potassium (87.7 mg, 0.65 eq) in portions over 5 minutes. The mixture was stirred overnight at room temperature under nitrogen. The reaction mixture was slowly transferred into a stirred NaHCO3 solution (sat., aq., ˜40 mL). The reaction was quenched with water and extracted with EA. The organic layer was washed with brine, dried over Na2SO4 and evaporated. The residue was purified using prep-HPLC with following conditions: Column: XBridge Prep OBD C18 Column, 30*150 mm, 5 μm; Mobile Phase A: Water (10 MMOL/L NH4HCO3), Mobile Phase B: ACN; Flow rate: 60 mL/min; Gradient: 30% B to 60% B in 8 min, 60% B; Wave Length: 254/220 nm; RT1 (min): 7.62. This resulted in 1-[(2-chloro-5-fluorophenyl)methyl]-6-[4-fluoro-6-(trifluoromethyl)-1H-1,3-benzodiazol-2-yl]piperidin-2-one (17.1 mg, 38.5 μmol, 4.34%) as a white solid. Lcms: Rt=1.212 min, m/z=444.10 (M+1)+. 1H NMR (400 MHz, DMSO-d6) δ 7.71 (1H, s), 7.35 (1H, dd, J=10.8, 1.5 Hz), 7.27 (2H, ddt, J=8.9, 5.9, 2.8 Hz), 7.12 (1H, t, J=9.1 Hz), 5.02-4.92 (21H, m), 4.00 (1H, d, J=15.7 Hz), 2.47 (21H, t, J=4.5 Hz), 2.24 (1H, dddd, J=14.1, 11.2, 5.7, 3.7 Hz), 2.13 (1H, dq, J=13.6, 4.3 Hz), 1.86-1.64 (2H, m).

### Example 10

### N-{[(2R,3S)-2-(2-chloro-5-fluorophenyl)-5-oxopyrrolidin-3-yl]methyl}-3-fluoro-5-(trifluoromethyl)benzamide (I-821)

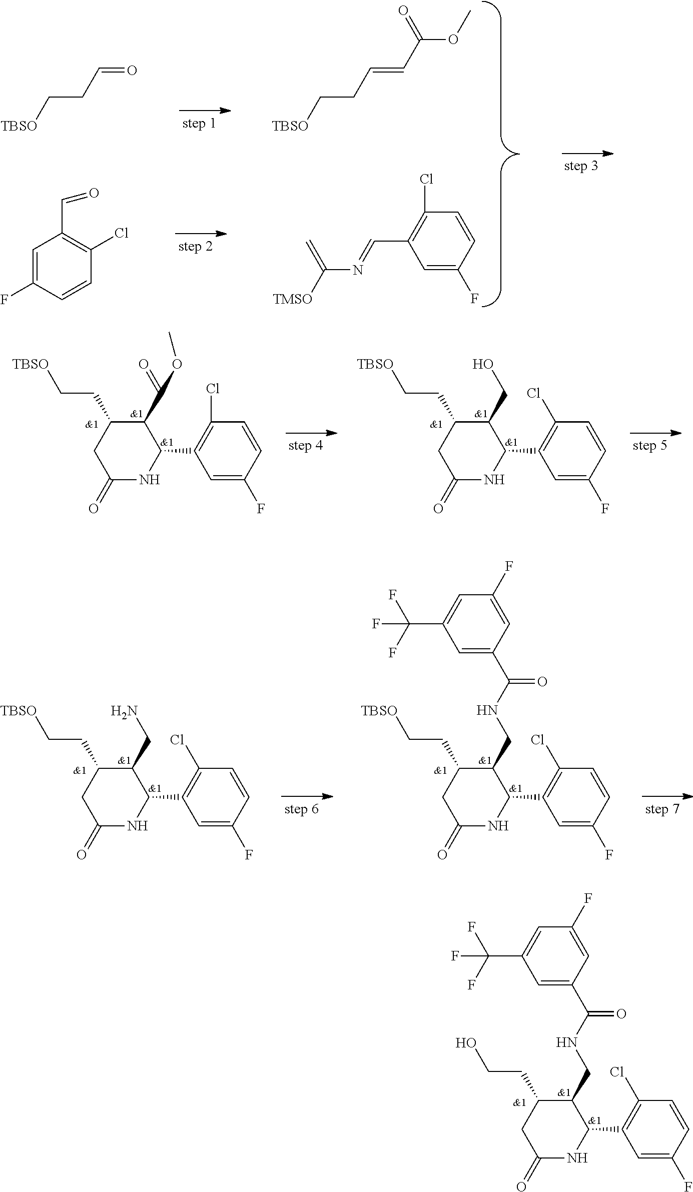

### Step 1. methyl (2E)-5-[(tert-butyldimethylsilyl)oxy]pent-2-enoate

To a flame dried 250 mL round-bottomed flask (RBF) charged with methyl 2-(dimethoxyphosphoryl)acetate (2.11 g, 1.1 eq) dissolved in 96 mL dry MeCN were added LiCl (533 mg, 1.2 eq) and then DIEA (1.63 g, 1.2 eq). The reaction was stirred at ambient temperature for 15 min and then 3-[(tert-butyldimethylsilyl)oxy]propanal (2 g, 10.6 mmol) dissolved in 4.6 mL dry MeCN was added. After stirring for 4 h the reaction was concentrated to approximately 50% volume, added to brine, and extracted thrice with EtOAc. Combined organic layers were dried over Na2SO4. The resulting mixture was concentrated under reduced pressure and was dissolved in DMF. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in methyl (2E)-5-[(tert-butyldimethylsilyl)oxy]pent-2-enoate (1.7 g, 6.95 mmol, 65.6%) as a white solid. Lcms: Rt=1.201 min, m/z=245.20 (M+1)+.

### Step 2. (E)-[(2-chloro-5-fluorophenyl)methylidene]({1-[(trimethylsilyl)oxy]ethenyl})amine

To a stirred solution of 2-chloro-5-fluorobenzaldehyde (3 g, 18.9 mmol) in dry THF (54 mL) was added LiHMDS (19 mL, 1M, 1.0 eq). After the mixture was stirred at room temperature for 0.5 h, TMSCl (2.06 g, 1.0 eq) was added dropwise. Then the temperature of the mixture was lowered to 0° C. on a cooling ice bath. To this mixture was added TEA (2.47 g, 1.3 eq) in one portion, followed by the dropwise addition of a solution of acetyl chloride (1.92 g, 1.3 eq) in Et2O (90 mL). The cooling bath was removed, and the mixture was stirred at room temperature for 1 h. Then the mixture was added toluene (20 mL). The mixture was quickly filtered on celite under nitrogen, and filtrate was concentrated under reduced pressure to give a mixture of (E)-[(2-chloro-5-fluorophenyl)methylidene]({1-[(trimethylsilyl)oxy]ethenyl})amine (2 g, 7.35 mmol, 38.9%) and toluene (20 mL). Lcms: Rt=2.222 min, m/z=272.15 (M+1)+.

### Step 3. methyl 4-{2-[(tert-butyldimethylsilyl)oxy]ethyl}-2-(2-chloro-5-fluorophenyl)-6-oxopiperidine-3-carboxylate

To a stirred solution of (E)-[(2-chloro-5-fluorophenyl)methylidene]({1-[(trimethylsilyl)oxy]ethenyl})amine (2.22 g, 8.18 mmol) in toluene (40 mL) was added methyl (2E)-5-[(tert-butyldimethylsilyl)oxy]pent-2-enoate (2 g, 8.18 mmol) and the resulting solution was stirred at 80° C. for 16 h under nitrogen atmosphere. The resulting mixture was concentrated under reduced pressure and was dissolved in DMF. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in methyl 4-{2-[(tert-butyldimethylsilyl)oxy]ethyl}-2-(2-chloro-5-fluorophenyl)-6-oxopiperidine-3-carboxylate (800 mg, 1.80 mmol, 22.0%) as a yellow solid. Lcms: Rt=1.238 min, m/z=444.10 (M+1)+

### Step 4. 4-{2-[(tert-butyldimethylsilyl)oxy]ethyl}-6-(2-chloro-5-fluorophenyl)-5-(hydroxymethyl)piperidin-2-one

To a stirred solution of methyl 4-{2-[(tert-butyldimethylsilyl)oxy]ethyl}-2-(2-chloro-5-fluorophenyl)-6-oxopiperidine-3-carboxylate (800 mg, 1.80 mmol) in EtOH (8 mL) was added NaBH4 (684 mg, 10 eq) at 0° C. The resulting mixture was stirred at room temperature for 1 h. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in 4-{2-[(tert-butyldimethylsilyl)oxy]ethyl}-6-(2-chloro-5-fluorophenyl)-5-(hydroxymethyl)piperidin-2-one (120 mg, 288 μmol, 16.0%) as a yellow solid. Lcms: Rt=1.154 min, m/z=416.10 (M+1)+.

### Step 5. 5-(aminomethyl)-4-{2-[(tert-butyldimethylsilyl)oxy]ethyl}-6-(2-chloro-5-fluorophenyl)piperidin-2-one

To a stirred solution of 4-{2-[(tert-butyldimethylsilyl)oxy]ethyl}-6-(2-chloro-5-fluorophenyl)-5-(hydroxymethyl)piperidin-2-one (90 mg, 216 μmol) and PPh3 (113 mg, 2 eq) in dry THF (3 mL) was added 2,3-dihydro-1H-isoindole-1,3-dione (38.1 mg, 259 μmol). Then the temperature of the mixture was lowered to 0° C. on a cooling ice bath. DIAD (3 mL) in dry THF (1 ml) was added dropwise. After the mixture was stirred at room temperature overnight under nitrogen atmosphere, N2H4·H2O (3 mL) in EtOH (3 mL) was added at room temperature. The resulting mixture was stirred at 60 C for 2 h. The reaction was quenched with water and extracted with EA. The organic laver was washed with brine, dried over Na2SO4 and evaporated. The residue was purified by Prep-TLC to afford 5-(aminomethyl)-4-{2-[(tert-butyldimethylsilyl)oxy]ethyl}-6-(2-chloro-5-fluorophenyl)piperidin-2-one (70 mg, 168 μmol, 78.1%) as a white solid. Lcms: Rt=0.923 min, m/z=415.10 (M+1)+

### Step 6. N-{[4-{2-[(tert-butyldimethylsilyl)oxy]ethyl}-2-(2-chloro-5-fluorophenyl)-6-oxopiperidin-3-yl]methyl}-3-fluoro-5-(trifluoromethyl)benzamide

To a solution of 3-fluoro-5-(trifluoromethyl)benzoic acid (32.8 mg, 158 μmol) in DCM (1 mL) was added Ghosez's reagent (21.1 mg, 1.3 eq). After stirring at room temperature for 1 h, TEA (43.6 mg, 3 eq) and 5-(aminomethyl)-4-{2[(tert-butyldimethylsilyl)oxy]ethyl}-6-(2-chloro-5-fluorophenyl)piperidin-2-one (60 mg, 144 μmol) were added and the resulting mixture was stirred at room temperature for 1 h. The reaction was quenched with water and extracted with EA. The organic layer was washed with brine, dried over Na2SO4 and evaporated. The residue was purified by Prep-TLC to afford N-{[4-{2-[(tert-butyldimethylsilyl)oxy]ethyl}-2-(2-chloro-5-fluorophenyl)-6-oxopiperidin-3-yl]methyl}-3-fluoro-5-(trifluoromethyl)benzamide (60 mg, 99.1 μmol, 68.8%) as a white solid. Lcms: Rt=1.334 min, m/z=605.10 (M+1)+.

### Step 7. N-{[2-(2-chloro-5-fluorophenyl)-4-(2-hydroxyethyl)-6-oxopiperidin-3-yl]methyl}-3-fluoro-5-(trifluoromethyl)benzamide

To a stirred solution of 5-(aminomethyl)-4-{2-[(tert-butyldimethylsilyl)oxy]ethyl}-6-(2-chloro-5-fluorophenyl)piperidin-2-one (60 mg, 144 μmol) was added HCl (4M, dioxane, 1 mL) and the resulting solution was stirred at room temperature for 1 h. The resulting mixture was concentrated under reduced pressure. The resulting mixture was concentrated under reduced pressure and was dissolved in DMF. The resulted solution was purified using prep-HPLC with following conditions: Column: XBridge Prep OBD C18 Column, 30×150 mm 5 um; Mobile Phase A: Water (10 mmoL/L NH4HCO3), Mobile Phase B: ACN; Flow rate: 60 mL/min; Gradient: 25% B to 50% B in 8 min; 220 nm, RT: 7.23 min. This resulted in N-{[2-(2-chloro-5-fluorophenyl)-4-(2-hydroxyethyl)-6-oxopiperidin-3-yl]methyl}-3-fluoro-5-(trifluoromethyl)benzamide (11.7 mg, 23.8 μmol, 24.0%) as a white amorphous solid. Lcms: Rt=0.931 min, m/z=491.10 (M+1)+. ¹H NMR (400 MHz, DMSO-d6) δ 8.63-8.55 (1H, m), 7.90 (2H, d, J=14.1 Hz), 7.86-7.76 (2H, m), 7.46 (1H, ddd, J=29.6, 8.8, 5.2 Hz), 7.30 (1H, ddd, J=31.3, 9.8, 3.1 Hz), 7.15 (1H, qd, J=8.4, 3.0 Hz), 4.91 (1H, dd, J=115.9, 6.0 Hz), 4.47 (1H, dt, J=29.1, 5.0 Hz), 3.53 (1H, q, J=6.1 Hz), 3.46-3.36 (1H, m), 3.32-3.19 (1H, m), 2.88-2.80 (1H, m), 2.67 (1H, dd, J=17.8, 6.1 Hz), 2.46 (1H, d, J=11.5 Hz), 2.28-2.00 (2H, m), 1.71-1.50 (2H, m).

### Example 11

### N-[(1R,2S,3S)-2-(2-chloro-5-fluorophenyl)-3-hydroxycyclohexyl]-3-fluoro-5-(trifluoromethyl)benzamide (I-823)

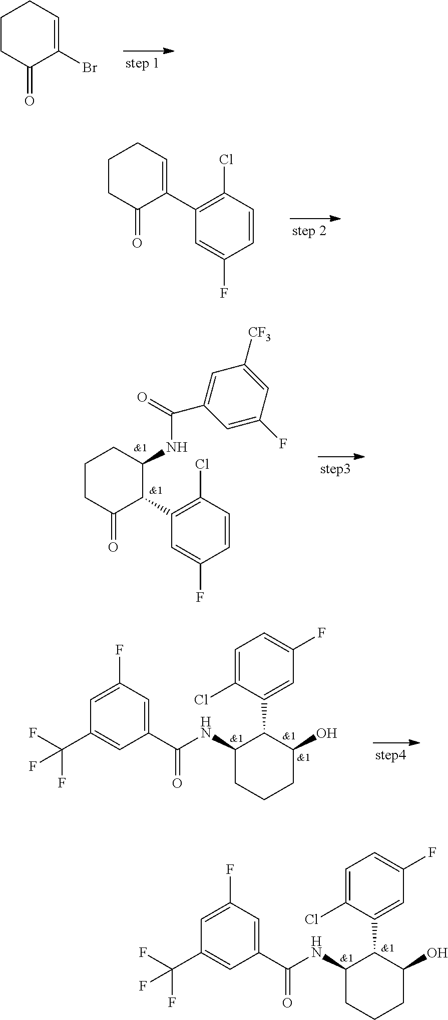 [SMILES: O=C1CCCC=C1Br ; O=C1CCCC=C1c1cc(F)ccc1Cl ; O=C(N[C@@H]1CCCC(=O)[C@H]1c1cc(F)ccc1Cl)c1cc(F)cc(C(F)(F)F)c1 ; O=C(N[C@@H]1CCC[C@H](O)[C@H]1c1cc(F)ccc1Cl)c1cc(F)cc(C(F)(F)F)c1 ; O=C(N[C@@H]1CCC[C@H](O)[C@H]1c1cc(F)ccc1Cl)c1cc(F)cc(C(F)(F)F)c1]

### Step 1. methyl (2E)-5-[(tert-butyldimethylsilyl)oxy]pent-2-enoate

To a stirred solution of 2-bromocyclohex-2-en-1-one (1 g, 5.71 mmol), (2-chloro-5-fluorophenyl)boronic acid (995 mg, 1 eq), NaHCO3 aq. (23 mL) in EtOH (23 mL) and DME (34.5 mL) were added Pd(PPh3)4 (660 mg, 0.1 eq) and the resulting solution was stirred at 85° C. for 6 h under nitrogen atmosphere. The reaction was quenched with water and extracted with EA. The organic layer was washed with brine, dried over Na2SO4 and evaporated. The residue was purified by Prep-TLC (PE/EA=2.5/1) to afford 2-(2-chloro-5-fluorophenyl)cyclohex-2-en-1-one (0.9 g, 4.00 mmol, 70.3%) as a white solid. Lcms: Rt=1.017 min, m/z=225.00 (M+1)+.

### Step 2. N-[(1R,2S)-2-(2-chloro-5-fluorophenyl)-3-oxocyclohexyl]-3-fluoro-5-(trifluoromethyl)benzamide

To a stirred solution of 2-(2-chloro-5-fluorophenyl)cyclohex-2-en-1-one (500 mg, 2.22 mmol) and 3-fluoro-5-(trifluoromethyl)benzamide (459 mg, 1 eq) in DMF was added Pd(Ph·CN)2Cl2 (8.39 mg, 0.01 eq) and the resulting solution was stirred at 60° C. for 48 h under nitrogen atmosphere. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in N-[(1R,2S)-2-(2-chloro-5-fluorophenyl)-3-oxocyclohexyl]-3-fluoro-5-(trifluoromethyl)benzamide (200 mg, 463 μmol, 20.8%) as a white solid. Lcms: Rt=1.120 min, m/z=431.95 (M+1)+

### Step 3. N-[(1R,2S,3S)-2-(2-chloro-5-fluorophenyl)-3-hydroxycyclohexyl]-3-fluoro-5-(trifluoromethyl)benzamide

To a stirred solution of N-[(1R,2S)-2-(2-chloro-5-fluorophenyl)-3-oxocyclohexyl]-3-fluoro-5-(trifluoromethyl)benzamide (190 mg, 440 μmol) in EtOH (2 mL) was added NaBH4 (166 mg, 10 eq) at 0° C. The resulting mixture was stirred at room temperature for 1 h. The reaction was quenched with water and extracted with EA. The organic layer was washed with brine, dried over Na2SO4 and evaporated. The residue was purified using prep-HPLC with following conditions: Column: XBridge Prep OBD C18 Column, 30×150 mm 5 um; Mobile Phase A: Water (10 mmoL/L NH4HCO3), Mobile Phase B: ACN; Flow rate: 60 mL/min; Gradient: 35% B to 65% B in 8 min, 65% B to 85% B in 9 min; 220 nm; RT: 6.92/8.38 min. This resulted in N-[(1R,2S,3S)-2-(2-chloro-5-fluorophenyl)-3-hydroxycyclohexyl]-3-fluoro-5-(trifluoromethyl)benzamide (100 mg, 230 μmol, 52.6%) as a white amorphous solid. Lcms: Rt=1.188 min, m/z=434.10 (M+1)+.

### Step 4. N-[2-(2-chloro-5-fluorophenyl)-3-hydroxycyclohexyl]-3-fluoro-5-(trifluoromethyl)benzamide

A solution of N-[2-(2-chloro-5-fluorophenyl)-3-hydroxycyclohexyl]-3-fluoro-5-(trifluoromethyl)benzamide (100 mg, 230 μmol) was purified using prep-HPLC with following conditions: Column: XBridge Prep OBD C18 Column, 30×150 mm 5 um; Mobile Phase A: Water (10 mmoL/L NH4HCO3), Mobile Phase B: ACN; Flow rate: 60 mL/min; Gradient: 35% B to 65% B in 8 min, 65% B to 85% B in 9 min; 220 nm; RT: 6.92/8.38 min. This resulted in N-[2-(2-chloro-5-fluorophenyl)-3-hydroxycyclohexyl]-3-fluoro-5-(trifluoromethyl)benzamide (41.8 mg, 96.3 μmol, 41.9%) as a white amorphous solid. Lcms: Rt=0.956 min, m/z=434.00 (M+1)+. 1H NMR (Chloroform-d, 400 MHz) δ 7.55 (1H, s), 7.50-7.34 (3H, m), 7.21 (1H, dd, J=9.4, 3.0 Hz), 6.93 (1H, ddd, J=8.9, 7.4, 3.0 Hz), 6.12 (1H d, J=8.9 Hz), 4.16 (1H, d, J=11.3 Hz), 3.97 (1H, s), 3.31 (1H, t, J=10.8 Hz), 2.25 (2H, ddt, J=11.7, 7.6, 3.8 Hz), 1.97 (1H, dp, J=12.6, 3.1, 2.6 Hz), 1.66 (1H, dt, J=13.2, 3.3 Hz), 1.61-1.51 (2H, m), 1.50-1.34 (1H, m).

Additional compounds prepared according to the methods of Example 11 are listed in Table 6 below. Corresponding ¹H NMR and mass spectrometry characterization for these compounds are described in Table 1. Certain compounds in Table 6 below were prepared with other compounds whose preparation is described further in the Examples herein.

TABLE 6
Additional Compounds
I-824

### Example 12

### trans-3-(2-chloro-5-fluorophenyl)-1-[(methylcarbamoyl)methyl]-5-oxo-N-[3-(trifluoromethyl)cyclohexyl]piperazine-2-carboxamide (I-825)

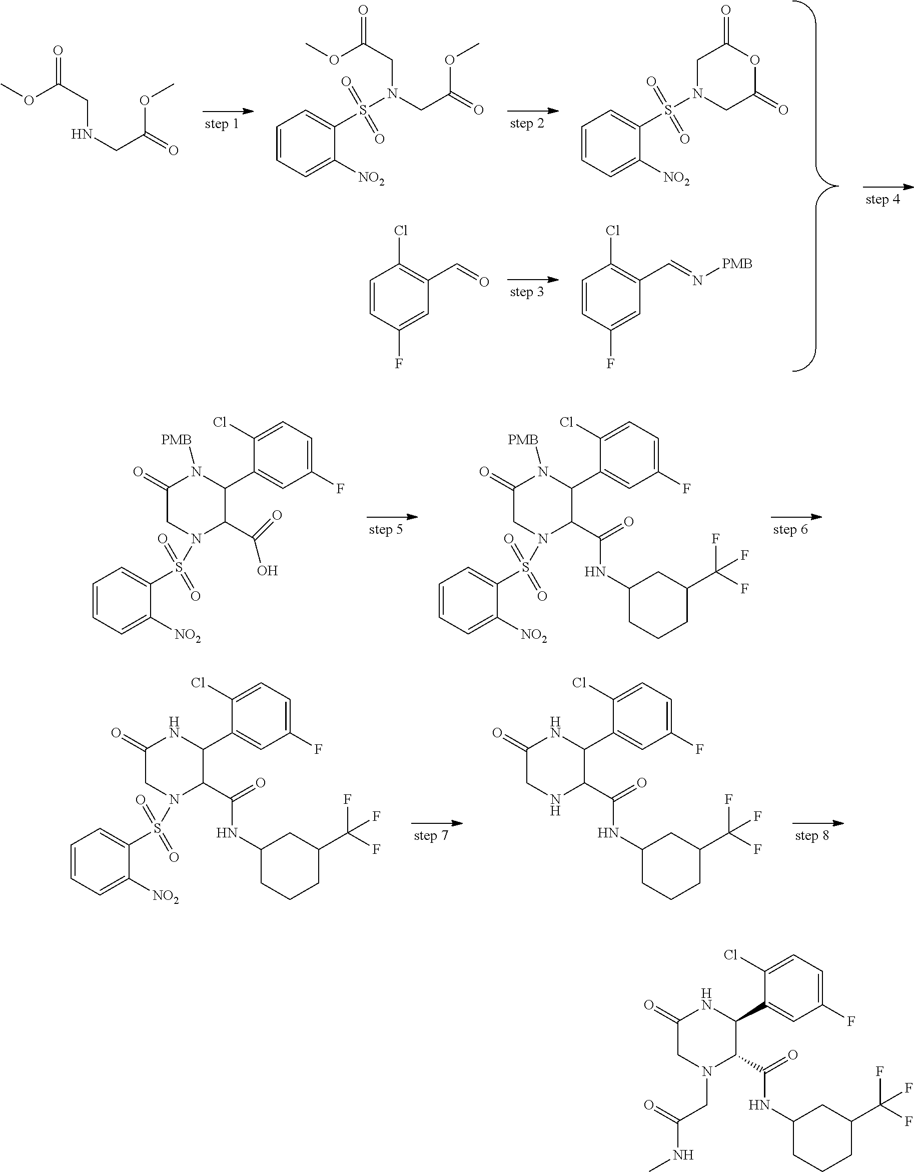

### Step 1. methyl 2-[N-(2-methoxy-2-oxoethyl)2-nitrobenzenesulfonamido]acetate

To a stirred solution of methyl 2-[(2-methoxy-2-oxoethyl)amino]acetate (2.43 g, 15.0 mmol) and 2-nitrobenzene-1-sulfonyl chloride (4.98 g, 22.5 mmol) in ACN (40 mL) was added Et₃N (1.8 mL) at 0° C. The resulting mixture was stirred at room temperature for 48 h. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in methyl 2-[N-(2-methoxy-2-oxoethyl)2-nitrobenzenesulfonamido]acetate (4 g, 11.5 mmol, 77.0%) as a white solid. Lcms: Rt=0.904 min, m/z=368.95 (M+23)+.

### Step 2. 4-(2-nitrobenzenesulfonyl)morpholine-2,6-dione

To a stirred solution of 2-[N-(carboxymethyl)2-nitrobenzenesulfonamido]acetic acid (3 g, 9.42 mmol) in dry EA (75 mL) was added TFAA (4.93 g, 2.5 eq) in dry EA at room temperature. The resulting mixture was stirred at room temperature for 24 h. After 24 h the resulting solution was concentrated in vacuo and the residue was thoroughly washed with cold petroleum ether (˜50 mL) to afford 4-(2-nitrobenzenesulfonyl)morpholine-2,6-dione (2.8 g, 9.32 mmol, 99.2%) as a white solid. Lcms: Rt=0.557 min, m/z=301.15 (M+1)+

### Step 3. (E)-[(2-chloro-5-fluorophenyl)methylidene][(4-methoxyphenyl)methyl]amine

To a stirred solution of 2-chloro-5-fluorobenzaldehyde (2 g, 12.6 mmol) and 1-(4-methoxyphenyl)methanamine (1.72 g, 1 eq) in DCM (2 mL) was added MgSO4 (3.02 g, 2 eq) at room temperature. The resulting mixture was stirred at room temperature for 24 h. The resulting mixture was filtered, the filter cake was washed with MeOH. The filtrate was concentrated under reduced pressure. This resulted in (E)-[(2-chloro-5-fluorophenyl)methylidene][(4-methoxyphenyl)methyl]amine (3 g, 10.8 mmol, 85.9%) as a white solid. Lcms: Rt=1.395 min, m/z=278.15 (M+1)+

### Step 4. 3-(2-chloro-5-fluorophenyl)-4-[(4-methoxyphenyl)methyl]-1-(2-nitrobenzenesulfonyl)-5-oxopiperazine-2-carboxylic Acid

To a stirred solution of 4-(2-nitrobenzenesulfonyl)morpholine-2,6-dione (2.8 g, 9.32 mmol) in dry toluene (10 mL) was added (E)-[(2-chloro-5-fluorophenyl)methylidene][(4-methoxyphenyl)methyl]amine (2.58 g, 9.32 mmol) at room temperature. The resulting mixture was stirred at 80° C. for 2 h. The resulting mixture was concentrated under reduced pressure and was dissolved in DMF. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in 3-(2-chloro-5-fluorophenyl)-4-[(4-methoxyphenyl)methyl]-1-(2-nitrobenzenesulfonyl)-5-oxopiperazine-2-carboxylic acid (1 g, 1.73 mmol, 18.5%) as a white solid. Lcms: Rt=1.059 min, m/z=578.00 (M+1)+.

### Step 5. 3-(2-chloro-5-fluorophenyl)-4-[(4-methoxyphenyl)methyl]-1-(2-nitrobenzenesulfonyl)-5-oxo-N-[3-(trifluoromethyl)cyclohexyl]piperazine-2-carboxamide

To a stirred solution of 3-(2-chloro-5-fluorophenyl)-4-[(4-methoxyphenyl)methyl]-1-(2-nitrobenzenesulfonyl)-5-oxopiperazine-2-carboxylic acid (500 mg, 865 μmol), 3-(trifluoromethyl)cyclohexan-1-amine (215 mg, 1.5 eq) and NaHCO3 (217 mg, 3 eq) in DMF (5.0 mL) was added HATU (490 mg, 1.5 eq) at room temperature. The resulting mixture was stirred at room temperature for 1 h. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in 3-(2-chloro-5-fluorophenyl)-4-[(4-methoxyphenyl)methyl]-1-(2-nitrobenzenesulfonyl)-5-oxo-N-[3-(trifluoromethyl)cyclohexyl]piperazine-2-carboxamide (600 mg, 825 μmol, 95.5%) as a white solid. Lcms: Rt=1.225 min, m/z=727.05 (M+1)+.

### Step 6. 3-(2-chloro-5-fluorophenyl)-1-(2-nitrobenzenesulfonyl)-5-oxo-N-[3-(trifluoromethyl)cyclohexyl]piperazine-2-carboxamide

To a stirred solution of 3-(2-chloro-5-fluorophenyl)-4-[(4-methoxyphenyl)methyl]-1-(2-nitrobenzenesulfonyl)-5-oxo-N-[3-(trifluoromethyl)cyclohexyl]piperazine-2-carboxamide (650 mg, 893 μmol) in ACN (26 mL) was added CAN (1.95 g, 4 eq) in H2O (13 mL) dropwise and the resulting solution was stirred at room temperature for 3 h. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in 3-(2-chloro-5-fluorophenyl)-1-(2-nitrobenzenesulfonyl)-5-oxo-N-[3-(trifluoromethyl)cyclohexyl]piperazine-2-carboxamide (370 mg, 609 μmol, 68.2%) as a white solid. Lcms: Rt=1.094 min, m/z=607.00 (M+1)+.

### Step 7. N-(2-(2-chloro-5-fluorophenylamino)-5-(2-hydroxypropan-2-yl)pyridin-3-yl)-3-fluoro-5-(trifluoromethyl)benzamide

To a stirred solution of 3-(2-chloro-5-fluorophenyl)-1-(2-nitrobenzenesulfonyl)-5-oxo-N-[3-(trifluoromethyl)cyclohexyl]piperazine-2-carboxamide (36 mg, 59.3 μmol) in ACN (0.5 mL) was added (phenylsulfanyl)sodium (54.8 mg, 7 eq) and the resulting solution was stirred at room temperature overnight. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in (2R,3S)-3-(2-chloro-5-fluorophenyl)-5-oxo-N-[3-(trifluoromethyl)cyclohexyl]piperazine-2-carboxamide (14.4 mg, 34.1 μmol, 57.6%) as a amorphous solid. Lcms: Rt=0.747 min, m/z=422.05 (M+1)+.

### Step 8. trans-3-(2-chloro-5-fluorophenyl)-1-[(methylcarbamoyl)methyl]-5-oxo-N-[3-(trifluoromethyl)cyclohexyl]piperazine-2-carboxamide

To a stirred solution of 3-(2-chloro-5-fluorophenyl)-5-oxo-N-[3-(trifluoromethyl)cyclohexyl]piperazine-2-carboxamide (50 mg, 118 μmol) and 2-bromo-N-methylacetamide (17.9 mg, 118 μmol) in ACN (1 mL) were added Cs2CO3 (22.7 mg, 1.0 eq) and the resulting solution was stirred at room temperature for 1 h. The resulting crude material was purified by HPLC (Column: Sunfire prep C18 column, 30*150, 5 um; Mobile Phase A: Water (0.1% FA), Mobile Phase B: ACN; Flow rate: 60 mL/min; Gradient: 25% B to 40% B in 11 min, Flow rate: 60 mL/min; Gradient: 20% B to 45% B in 8 min; 220 nm; RT: 11.25 min). This resulted in trans-3-(2-chloro-5-fluorophenyl)-1-[(methylcarbamoyl)methyl]-5-oxo-N-[3-(trifluoromethyl)cyclohexyl]piperazine-2-carboxamide (7.8 mg, 15.8 μmol, 13.4%) as an amorphous solid. Lcms: Rt=0.785 min, m/z=493.15 (M+1)+. ¹H NMR (400 MHz, DMSO-d6) δ 7.45 (2H, dd, J=8.8, 5.0 Hz), 7.21 (1H, d, J=8.2 Hz), 7.14-7.05 (1H, m), 6.84 (1H, dd, J=8.7, 2.9 Hz), 5.65 (1H, s), 4.81 (1H, d, J=16.8 Hz), 3.92 (1H, dd, J=12.0, 3.8 Hz), 3.76 (1H, s), 3.61 (2H, d, J=6.5 Hz), 3.06 (1H, d, J=16.8 Hz), 2.89 (3H, d, J=4.7 Hz), 2.26 (2H, d, J=12.3 Hz), 2.11-1.93 (3H, m), 1.47 (1H, t, J=13.4 Hz), 1.38-1.16 (4H, m).

### Example 13

### 3-hydroxy-N-(2-(o-tolylamino)quinolin-3-yl)indoline-1-carboxamide (I-828)

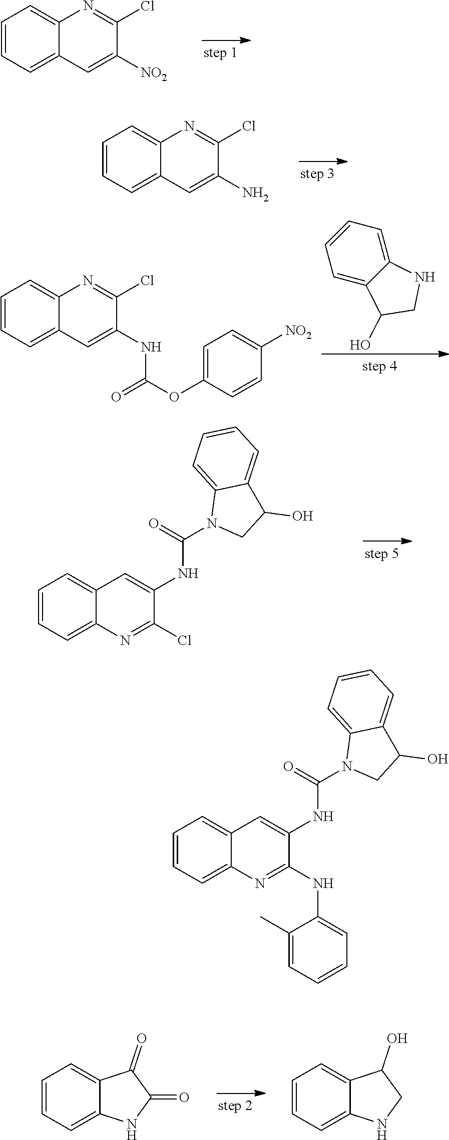

### Step 1. 2-chloroquinolin-3-amine

A round bottomed flask was charged with 2-chloro-3-nitroquinoline (208 mg, 997 μmol), iron (278 mg, 4.98 mmol), ammonium chloride (212 mg, 3.98 mmol) and a stirbar. EtOH (3 mL) and water (2 mL) were added, and the solution was stirred at 80° C. for 1.5 h. Filtered the mixture, and the filtrate was extracted with EA. The organic layer was washed with brine, dried over Na2SO4 and evaporated. This resulted in 2-chloroquinolin-3-amine (150 mg, 839 μmol, 84.2%) as a yellow solid. m/z (ES+) [M+Na]+=178.95; HPLC tR=0.747 min.

### Step 2. indolin-3-ol

A round bottomed flask was charged with 2,3-dihydro-1H-indole-2,3-dione (1.3 g, 8.83 mmol) in tetrahydrofuran (30 mL). Lithium aluminum hydride (1.67 g, 44.1 mmol) was added at 0° C., and the solution was stirred at rt for 1 h. The solution was diluted with ether and cooled to 0° C. Added water (1.67 ml) slowly, then added NaOH (c=15%, 1.67 ml), and water (5.01 ml). Warmed the mixture to rt, and the mixture was stirred at rt for 15 min. The mixture was dried over MgSO4 and stirred for 15 min. The resulted solution was evaporated and purified using C18 flash chromatography with the following conditions (Mobile Phase A: 10 mmol/L NH4HCO3, Mobile Phase B: ACN; Flow rate: 60 mL/min; Gradient: 0% B to 100% B in 40 min; 254/220 nm). This resulted in 2,3-dihydro-1H-indol-3-ol (300 mg, 2.21 mmol, 25.2%) as an off-white solid. m/z (ES+) [M+H]+=118.25; HPLC tR=0.498 min.

### Step 3. 4-nitrophenyl (2-chloroquinolin-3-yl)carbamate

A round bottomed flask was charged with 2-chloroquinolin-3-amine (100 mg, 559 μmol), 4-nitrophenyl carbonochloridate (168 mg, 838 μmol) and a stirbar. Tetrahydrofuran (3 mL) was added, and the solution was stirred at 70° C. for 1 h. The mixture was used directly to next step. m/z (ES+) [M+H]+=344.00; HPLC tR=0.997 min.

### Step 4. N-(2-chloroquinolin-3-yl)-3-hydroxyindoline-1-carboxamide

A round bottomed flask was charged with 4-nitrophenyl N-(2-chloroquinolin-3-yl)carbamate (160 mg, 465 μmol), 2,3-dihydro-1H-indol-3-ol (156 mg, 1.16 mmol), triethylamine (140 mg, 3 eq) and a stirbar. Tetrahydrofuran (4 mL) was added, and the solution was stirred at 70° C. for 1 h. The resulted solution was evaporated and purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in N-(2-chloroquinolin-3-yl)-3-hydroxy-2,3-dihydro-1H-indole-1-carboxamide (150 mg, 441 μmol, 95.5%) as an off-white solid. m/z (ES+) [M+H]+=340.05, HPLC tR=0.813 min.

### Step 5. 3-hydroxy-N-(2-(o-tolylamino)quinolin-3-yl)indoline-1-carboxamide

A round bottomed flask was charged with N-(2-chloroquinolin-3-yl)-3-hydroxyindoline-1-carboxamide (65 mg, 1 Eq, 0.19 mmol), o-toluidine (31 mg, 1.5 Eq, 0.29 mmol), Brettphos Pd G3 (17 mg, 0.1 Eq, 19 μmol), LiHMDS (0.19 g, 6 Eq, 1.1 mmol), and a stirbar. Toluene (4 mL) was added, and the solution was stirred at 100° C. for 2 hours under N2. The reaction was quenched with water and extracted with EA. The organic layer was washed with brine, dried over Na2SO4 and evaporated. Then purified the crude product by Prep-TLC (EA:PE=1:1). The resulting material was further purified using prep-HPLC with following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 35% B to 60% B in 8 min, 60% B; Wave Length: 254 nm; RT1: 7.67 min. Lyophilization yielded 3-hydroxy-N-(2-(o-tolylamino)quinolin-3-yl)indoline-1-carboxamide (9.2 mg, 22 μmol, 12%) as an off-white amorphous solid. m/z=411.30 (M+1)+. ¹H NMR (DMSO-d6, 400 MHz) 8.57-8.53 (1H, m), 8.11 (1H, s), 8.05 (1H, s), 7.93 (2H, dd, J=16.1, 8.0 Hz), 7.80-7.73 (1H, m), 7.59-7.46 (2H, m), 7.39 (1H, d, J=7.3 Hz), 7.33-7.19 (4H, m), 7.02 (2H, dtd, J=18.9, 7.4, 1.2 Hz), 5.73 (1H, d, J=6.0 Hz), 5.29 (1H, ddd, J=9.0, 6.0, 3.3 Hz) 4.35 (1H, dd, J=11.1, 7.9 Hz), 4.04 (1H, dd, J=11.1, 3.4 Hz), 2.25 (3H, s).

### Example 14

### 5-((2-chloro-5-fluorophenyl)amino)-4-(3-fluoro-5-(trifluoromethyl)benzamido)-N-methylthiophene-2-carboxamide (I-827)

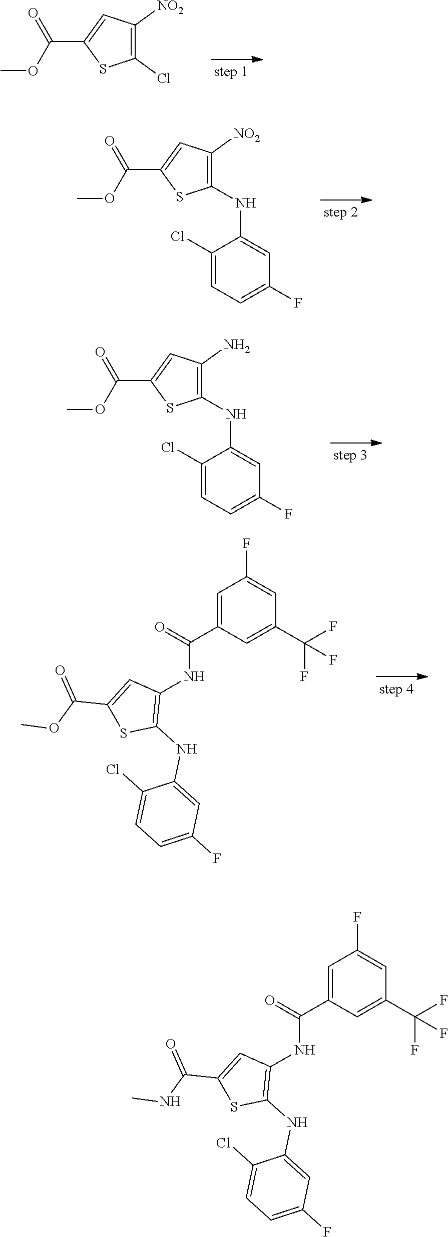

### Step 1. methyl 5-((2-chloro-5-fluorophenyl)amino)-4-nitrothiophene-2-carboxylate

A round bottomed flask was charged with methyl 5-chloro-4-nitrothiophene-2-carboxylate (500 mg, 2.25 mmol), 2-chloro-5-fluoroaniline (327 mg, 2.25 mmol), potassium carbonate (630 mg, 2 eq), and a stirbar. NMP (2.5 mL) was added, and the solution was stirred at 140° C. overnight. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in methyl 5-[(2-chloro-5-fluorophenyl)amino]-4-nitrothiophene-2-carboxylate (70 mg, 211 μmol, 9.40%) as a yellow solid. m/z (ES+) [M+H]+=330.90, HPLC tR=1.240 min.

### Step 2. methyl 5-((2-chloro-5-fluorophenyl)amino)-4-nitrothiophene-2-carboxylate

A round bottomed flask was charged with methyl 5-[(2-chloro-5-fluorophenyl)amino]-4-nitrothiophene-2-carboxylate (55 mg, 166 μmol), Pd/C (10 mg) and a stirbar. EA (10 mL) was added, and the solution was stirred at rt for 2 d under H2. Filtered the mixture. The filtrate was evaporated to give the product methyl 4-amino-5-[(2-chloro-5-fluorophenyl)amino]thiophene-2-carboxylate (35 mg, 116 μmol, 70.1%) as a yellow solid. m/z (ES+) [M±H]+=300.90; HPLC tR=0.938 min.

### Step 3. methyl 5-((2-chloro-5-fluorophenyl)amino)-4-(3-fluoro-5-(trifluoromethyl)benzamido)thiophene-2-carboxylate

A round bottomed flask was charged with 3-fluoro-5-(trifluoromethyl)benzoic acid (22.6 mg, 109 μmol), ((1-chloro-2-methyprop-1-en-1-yl)dimethylamine (17.2 mg, 129 μmol) and a stirbar. Dichloromethane (4 mL) was added, and the solution was stirred at rt for 1 h. Then add triethylamine (30.2 mg, 299 μmol) and methyl 4-amino-5-[(2-chloro-5-fluorophenyl)amino]thiophene-2-carboxylate (30 mg, 99.7 μmol), and the solution was stirred at rt for 1 h. The reaction was quenched with water and extracted with EA. The organic layer was washed with brine, dried over Na2SO4 and evaporated. This resulted in the crude product methyl 5-[(2-chloro-5-fluorophenyl)amino]-4-[3-fluoro-5-(trifluoromethyl) benzamido]thiophene-2-carboxylate (70 mg, 60%) as a brown oil. m/z (ES+) [M+H]+=490.90; HPLC tR=1.365 min.

### Step 4. 5-((2-chloro-5-fluorophenyl)amino)-4-(3-fluoro-5-(trifluoromethyl)benzamido)-N-methylthiophene-2-carboxamide

A round bottomed flask was charged with methyl 5-[(2-chloro-5-fluorophenyl)amino]-4-[3-fluoro-5-(trifluoromethyl)benzamido]thiophene-2-carboxylate (65 mg, 132 μmol) in MeOH (5 mL) and a stirbar. Methanamine (40% in water, 20 mL) was added, and the solution was stirred at rt overnight. The reaction was quenched with water and extracted with EA. The organic layer was washed with brine, dried over Na2SO4 and evaporated. The resulting crude material was purified using prep-HPLC. Lyophilization yielded 5-[(2-chloro-5-fluorophenyl)amino]-4-[3-fluoro-5-(trifluoromethyl)benzamido]-N-methylthiophene-2-carboxamide (3.3 mg, 6.73 μmol, 5.10%) as an off-white solid, m/z (ES+) [M+H]+=490.05. ¹H NMR (DMSO-d6, 400 MHz) 10.30 (1H, s), 8.50 (1H, d, J=4.9 Hz), 8.10 (1H, s), 8.00-7.92 (3H, m), 7.82 (1H, s), 7.41 (1H, dd, J=8.8, 5.8 Hz), 6.72 (1H, dd, J=11.1, 2.9 Hz), 6.66 (1H, td, J=8.3, 2.9 Hz), 2.77 (3H, d, J=4.5 Hz).

### Example 15

### 4-{2-[4-fluoro-6-(trifluoromethyl)-1H-1,3-benzodiazol-2-yl]phenyl}-1,2,3,4-tetrahydroisoquinolin-3-one (I-820)

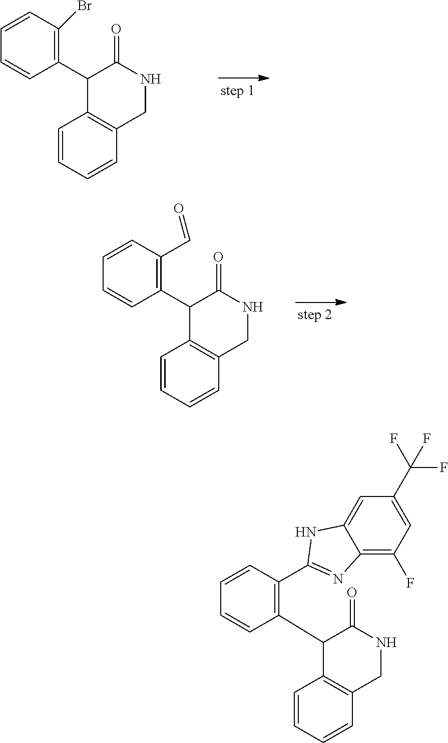 [SMILES: O=C1NCc2ccccc2C1c1ccccc1Br ; O=Cc1ccccc1C1C(=O)NCc2ccccc21 ; O=C1NCc2ccccc2C1c1ccccc1-c1nc2c(F)cc(C(F)(F)F)cc2[nH]1]

### Step 1. 2-(3-oxo-1,2,3,4-tetrahydroisoquinolin-4-yl)benzaldehyde

To a stirred solution of 4-(2-bromophenyl)-1,2,3,4-tetrahydroisoquinolin-3-one (70 mg, 231 μmol) in THF (1 mL) was added n-BuLi (2.5M, 0.19 mL, 2.0 eq) dropwise at −78° C. under nitrogen atmosphere. After the mixture was stirred at −78° C. for 1 h, DMF (67.4 mg, 4 eq) was added dropwise, and the mixture was stirred at −78° C. for 1 h under nitrogen atmosphere. The reaction was quenched with NH4Cl aq. and extracted with EA. The organic layer was washed with brine, dried over Na2SO4 and evaporated. The residue was purified by Prep-TLC to afford 2-(3-oxo-1,2,3,4-tetrahydroisoquinolin-4-yl)benzaldehyde (20 mg, 79.5 μmol, 34.4%) as a white solid. Lcms: Rt=0.984 min, m/z=252.00 (M+1)+.

### Step 2. 4-{2-[4-fluoro-6-(trifluoromethyl)-1H-1,3-benzodiazol-2-yl]phenyl}-1,2,3,4-tetrahydroisoquinolin-3-one

To a stirred solution of 2-(3-oxo-1,2,3,4-tetrahydroisoquinolin-4-yl)benzaldehyde (15 mg, 59.6 μmol), 3-fluoro-5-(trifluoromethyl)benzene-1,2-diamine (11.5 mg, 1 eq) in DMF (1.0 mL) and H2O (0.1 mL) was added KHSO5 (5.88 mg, 0.65 eq) at 0° C. under nitrogen atmosphere. The resulting mixture was stirred at room temperature for 1 h under nitrogen atmosphere. The reaction was quenched with water and extracted with EA. The organic layer was washed with brine, dried over Na2SO4 and evaporated. The residue was purified by Prep-TLC (PE/EA=1/2) and evaporated. The residue was purified using prep-HPLC with following conditions: Column: Sunfire prep C18 column, 30*150, 5 um; Mobile Phase A: Water (0.1% FA), Mobile Phase B: ACN; Flow rate: 60 mL/min; Gradient: 45% B to 70% B in 8 min; 220 nm; RT: 7.83 min. This resulted in 4-{2-[4-fluoro-6-(trifluoromethyl)-1H-1,3-benzodiazol-2-yl]phenyl}-1,2,3,4-tetrahydroisoquinolin-3-one (2.9 mg, 6.81 μmol, 11.4%) as an amorphous solid. Lcms: Rt=0.959 min, m/z=426.10 (M+1)+. ¹H NMR (Chloroform-d, 400 MHz): 14.48 (1H, s), 8.17 (1H, d, J=7.7 Hz), 7.61-7.89 (1H, m), 7.44 (1H, t, J=7.5 Hz), 7.38 (2H, d, J=8.0 Hz), 7.34 (2H, dd, J=8.9, 1.9 Hz), 7.24 (1H, t, J=7.0 Hz), 6.91-6.83 (2H, m), 6.58 (1H, s), 5.29 (1H, s), 5.08 (1H, d, J=16.3 Hz), 4.79-4.70 (1H, m).

### Example 16

Selected compounds of the present disclosure were tested in an ADP-Glo Biochemical PIK3CA Kinase Assay. Compounds to be assayed were plated in 16 doses of 1:2 serial dilutions (20 nL volume each well) on a 1536-well plate, and the plate warmed to room temperature. PIK3CA enzyme (e.g. H1047R, E542K, E545K, or wild-type) (1 μL of 2 nM solution in Enzyme Assay Buffer (comprising 50 mM HEPES pH 7.4, 50 mM NaCl, 6 mM MgCl₂, 5 mM DTT and 0.03% CHAPS)) was added and shaken for 10 seconds and preincubated for 30 minutes. To the well was added 1 μL of 200 μM ATP and 20 μM of diC8-PIP2 in Substrate Assay Buffer (50 mM HEPES pH7.4, 50 mM NaCl, 5 mM DTT and 0.03% CHAPS) to start the reaction, and the plate was shaken for 10 seconds, then spun briefly at 1500 rpm, and then incubated for 60 minutes at room temperature. The reaction was stopped by adding 2 μL of ADP-Glo reagent (Promega), and spinning briefly at 1500 rpm, and then incubating for 40 minutes. ADP-Glo Detection reagent (Promega) was added and the plate spun briefly at 1500 rpm, then incubated for 30 minutes. The plate was read on an Envision 2105 (Perkin Elmer), and the IC₅₀ values were calculated using Genedata software.

Results of the ADP-Glo Biochemical PIK3CA Kinase Assay using H1047R PIK3CA enzyme are presented in Table 1. Compounds having an IC₅₀ less than or equal to 100 nM are represented as “A”; compounds having an IC₅₀ greater than 100 nM but less than or equal to 500 nM are represented as “B”; compounds having an IC₅₀ greater than 500 nM but less than or equal to 1 μM are represented as “C”; compounds having an IC₅₀ greater than 1 μM but less than or equal to 10 μM are represented as “D”; and compounds having an IC₅₀ greater than 10 μM but less than or equal to 100 μM are represented as “E”.

### Example 17

Selected compounds of the present disclosure were tested in a MCF10A Cell-Based PIK3CA Kinase Assay, namely the CisBio Phospho-AKT (Ser473) HTRF assay, to measure the degree of PIK3CA-mediated AKT phosphorylation. MCF10A cells (immortalized non-transformed breast cell line) overexpressing hotspot PIK3CA mutations (including H1047R, E542K, and E545K mutations) were used. Cells were seeded at 5,000 cells per well in DMEM/F12 (Thermo Fisher Scientific) supplemented with 0.5 mg/mL hydrocortisone, 100 ng/mL Cholera Toxin, 10 μg/mL insulin, and 0.5% horse serum. Once plated, cells were placed in a 5% CO₂, 37° C. incubator to adhere overnight.

The following day, compounds were added to the cell plates in 12 doses of 1:3 serial dilutions. The dose response curves were run in duplicate. Compound addition was carried out utilizing an Echo 55 Liquid Handler acoustic dispenser (Labcyte). The cell plates were incubated for 2 hours in a 5% CO₂, 37° C. incubator. Following compound incubation, the cells were lysed for 60 min at room temperature. Finally, a 4-hour incubation with the HTRF antibodies was performed at room temperature. All reagents, both lysis buffer and antibodies, were used from the CisBio pAKT S473 HTRF assay kit, as per the manufacturers protocol. Plates were read on an Envision 2105 (Perkin Elmer), and the IC₅₀ values were calculated using Genedata software.

Results of the MCF10A Cell-Based PIK3CA Kinase Assay are presented in Table 1. Compounds having an IC₅₀ less than or equal to 1 μM are represented as “A”; compounds having an IC₅₀ greater than 1 μM but less than or equal to 5 μM are represented as “B”; compounds having an IC₅₀ greater than 5 μM but less than or equal to 10 μM are represented as “C”; compounds having an IC₅₀ greater than 10 μM but less than or equal to 36 μM are represented as “D”; and compounds having an IC₅₀ greater than 36 μM but less than or equal to 100 μM are represented as “E”.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1-33. (canceled)

34. A compound selected from the group consisting of: 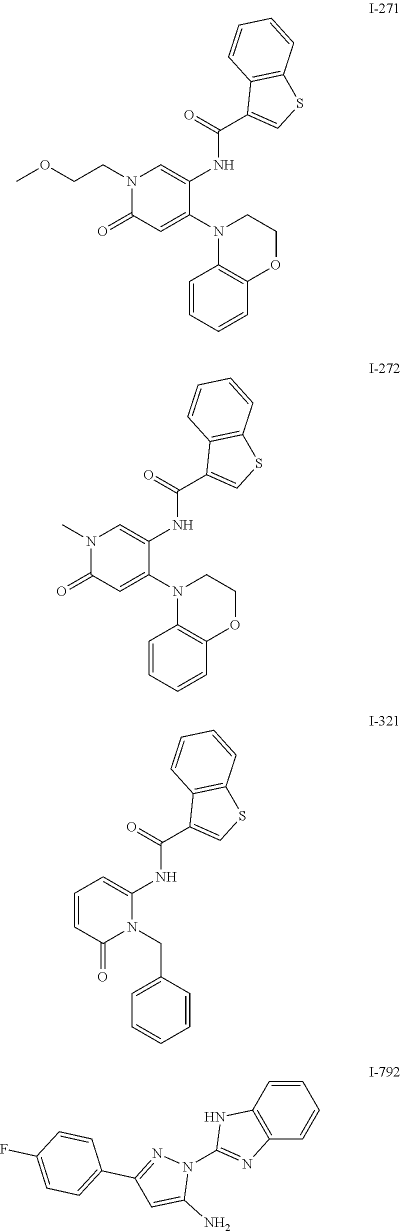 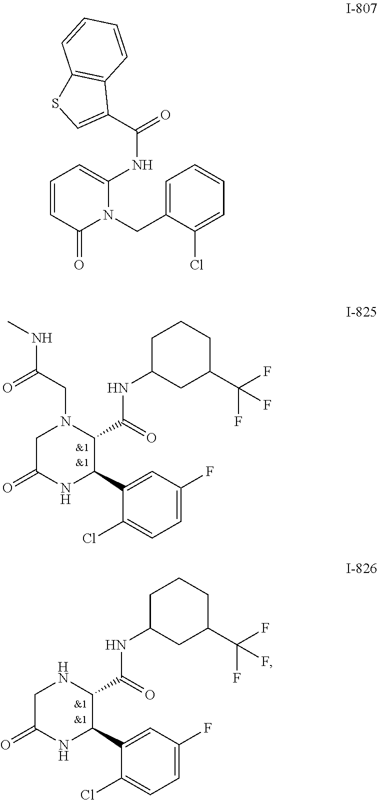 [SMILES: O=C(Nc1cccc(=O)n1Cc1ccccc1Cl)c1csc2ccccc12 ; CNC(=O)CN1CC(=O)N[C@H](c2cc(F)ccc2Cl)[C@H]1C(=O)NC1CCCC(C(F)(F)F)C1 ; O=C1CN[C@H](C(=O)NC2CCCC(C(F)(F)F)C2)[C@@H](c2cc(F)ccc2Cl)N1]
or a pharmaceutically acceptable salt thereof.

35. A pharmaceutical composition, comprising the compound of claim 34, or a pharmaceutically acceptable salt thereof, and a pharmaceutically acceptable carrier.

36. A method of inhibiting PI3Kα signaling activity in a subject, comprising administering a therapeutically effective amount of the compound of claim 34, or a pharmaceutically acceptable salt thereof, to the subject.

37. A method of treating a PI3Kα-mediated disorder in a subject, comprising administering a therapeutically effective amount of the compound of claim 34, or a pharmaceutically acceptable salt thereof, to the subject.

38. A method of treating a cellular proliferative disease in a subject, comprising administering a therapeutically effective amount of the compound of claim 34, or a pharmaceutically acceptable salt thereof, to the subject.

39. The method of claim 38, wherein the cellular proliferative disease is cancer.

40. The method of claim 39, wherein the cancer is breast cancer.

41. The method of claim 39, wherein the cancer is ovarian cancer.

42. The method of claim 41, wherein the ovarian cancer is clear cell ovarian cancer.

43. The method om claim 36, wherein the subject has PI3Kα containing at least one of the following mutations: H1047R, E542K, and E545K.

44. The method of claim 37, wherein the subject has PI3Kα containing at least one of the following mutations: H1047R, E542K, and E545K.

45. The method of claim 38, wherein the subject has PI3Kα containing at least one of the following mutations: H1047R, E542K, and E545K.
